(19) United States
(12) Patent Application Publication
(10) Pub. No.: US 2023/0416248 A1
(43) Pub. Date: Dec. 28, 2023

(54) COMPOUNDS AND USES THEREOF

(71) Applicant: Foghorn Therapeutics Inc., Indianapolis, IN (US)

(72) Inventors: Kevin J. WILSON, Roslindale, MA (US); Solymar NEGRETTI, Watertown, MA (US); Shawn E.R. SCHILLER, Haverhill, MA (US); Rishi G. VASWANI, Lexington, MA (US); David S. HUANG, Cambridge, MA (US); Johannes H. VOIGT, Cambridge, MA (US)

(21) Appl. No.: 18/252,228

(22) PCT Filed: Nov. 10, 2021
(86) PCT No.: PCT/US2021/058865

§ 371 Date: May 9, 2023

Related U.S. Application Data
(60) Provisional application No. 63/112,126, filed on Nov. 10, 2020.

(51) Int. Cl. C07D 471/04; C07D 519/00; A61P 35/00

(52) U.S. Cl. CPC C07D 471/04; C07D 519/00; A61P 35/00

(57) ABSTRACT

The present disclosure features compounds useful for the treatment of BAF complex-related disorders.

### BACKGROUND

The invention relates to compounds useful for modulating BRG1- or BRM-associated factors (BAF) complexes. In particular, the invention relates to compounds useful for treatment of disorders associated with BAF complex function.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY

The present invention features compounds useful for modulating a BAF complex. In some embodiments, the compounds are useful for the treatment of disorders associated with an alteration in a BAF complex, e.g., a disorder associated with an alteration in one or both of the BRG1 and BRM proteins. The compounds of the invention, alone or in combination with other pharmaceutically active agents, can be used for treating such disorders.

In one aspect, the invention provides a compound having the structure:

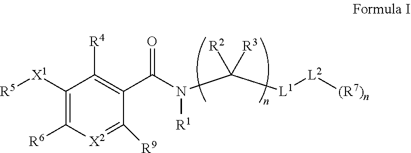

where
m is 0, 1, 2, or 3;
n is 0, 1, 2, 3, or 4;
X¹ is —S—, —SO—, —SO₂—, or —S(O)(NH)—;
X² is N or CR⁸;
R¹ is hydrogen or optionally substituted C₁-C₆ alkyl;
each R² and each R³ are independently hydrogen, optionally substituted C₁-C₆ alkyl, or optionally substituted C₁-C₆ heteroalkyl;
L¹ is optionally substituted 9- or 10-membered bicyclic heterocyclyl or optionally substituted 9- or 10-membered bicyclic heteroaryl;
L² is absent, optionally substituted C₃-C₁₀ cycloalkyl, optionally substituted C₆-C₁₀ aryl, optionally substituted 5- to 14-membered heteroaryl, or optionally substituted 4- to 14-membered heterocyclyl;
R⁴ is hydrogen, halo, optionally substituted C₁-C₆ alkyl, or optionally substituted C₃-C₁₀ cycloalkyl;
R⁵ is optionally substituted C₁-C₆ alkyl, optionally substituted C₁-C₆ heteroalkyl, or optionally substituted amino, and R⁶ is hydrogen, halo, cyano, optionally substituted C₁-C₆ alkyl, optionally substituted C₂-C₆ alkenyl, or optionally substituted C₃-C₁₀ cycloalkyl; or R⁵ and R⁶, together with the atoms to which they are attached, combine to form an optionally substituted 5- to 8-membered heterocyclyl;
each R⁷ is independently optionally substituted C₁-C₆ alkyl, optionally substituted C₁-C₆ heteroalkyl, halo, optionally substituted C₃-C₁₀ cycloalkyl, optionally substituted C₃-C₁₀ cycloalkyl C₁-C₆ alkyl, optionally substituted 5- to 14-membered heteroaryl, optionally substituted 4- to 14-membered heterocyclyl, —N(R^{7A})₂, or —OR^{7A}, wherein each R^{7A} is independently H, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted 5- to 10-membered heteroaryl, or optionally substituted 4- to 10-membered heterocyclyl, or two gamine/R^{7A} groups, together with the atom to which they are attached, combine to form optionally substituted 5- to 10-membered heteroaryl or optionally substituted 4- to 10-membered heterocyclyl; or two geminal R⁷ groups, together, with the atom to which they are attached, combine to form carbonyl;
R⁸ is hydrogen, halo, optionally substituted C₁-C₆ alkyl, or optionally substituted C₃-C₁₀ cycloalkyl; and
R⁸ is hydrogen or halo;
or a pharmaceutically acceptable salt thereof.

In some embodiments, the variables for the compound of Formula I are as follows:
m is 0, 1, 2, or 3;
n is 0, 1, 2, 3, or 4;
X¹ is S, SO, SO₂, or S(O)(NH);
X² is N or CR⁸;
R¹ is hydrogen or optionally substituted C₁-C₆ alkyl;
each R² and each R³ are independently hydrogen, optionally substituted C₁-C₆ alkyl, or optionally substituted C₁-C₆ heteroalkyl;
L¹ is optionally substituted 9- or 10-membered bicyclic heterocyclyl or optionally substituted 9- or 10-membered bicyclic heteroaryl;
L² is absent, optionally substituted C₃-C₁₀ cycloalkyl, optionally substituted C₆-C₁₀ aryl, optionally substituted 5- to 10-membered heteroaryl, or optionally substituted 4- to 10-membered heterocyclyl;
R⁴ is hydrogen, halo, optionally substituted C₁-C₆ alkyl, or optionally substituted C₃-C₁₀ cycloalkyl;
R⁵ is optionally substituted C₁-C₆ alkyl, optionally substituted C₁-C₆ heteroalkyl, or optionally substituted amino, and R⁶ is hydrogen, halo, cyano, optionally substituted C₁-C₆ alkyl, or optionally substituted C₃-C₁₀ cycloalkyl; or R⁵ and R⁶, together with the atoms to which they are attached, combine to form an optionally substituted 5- to 8-membered heterocyclyl;
each R⁷ is independently optionally substituted C₁-C₆ alkyl, optionally substituted C₁-C₆ heteroalkyl, halo, optionally substituted C₃-C₁₀ cycloalkyl, optionally substituted 5- to 10-membered heteroaryl, optionally substituted 4- to 10-membered heterocyclyl, —N(R^{7A})₂, or —OR^{7A}, where each R^{7A} is independently H, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted 5- to 10-membered heteroaryl, or optionally substituted 4- to 10-membered heterocyclyl, or two geminal R^{7A} groups, together with the atom to which they are attached, combine to form optionally substituted 5- to 10-membered heteroaryl or optionally substituted 4- to 10-membered heterocyclyl;
R⁸ is hydrogen, halo, optionally substituted C₁-C₆ alkyl, or optionally substituted C₃-C₁₀ cycloalkyl; and
R⁹ is hydrogen;
or a pharmaceutically acceptable salt thereof.

In some embodiments, L² is absent, optionally substituted C₃-C₁₀ cycloalkyl, optionally substituted C₆-C₁₀ aryl, optionally substituted 5- to 10-membered heteroaryl, or optionally substituted 4- to 10-membered heterocyclyl.

In some embodiments, each R¹ is independently optionally substituted C₁-C₆ alkyl, optionally substituted C₁-C₆ heteroalkyl, halo, optionally substituted C₃-C₁₀ cycloalkyl, optionally substituted 5- to 10 membered heteroaryl, optionally substituted 4- to 10-membered heterocyclyl, —N(R^{7A})₂, or —OR^{7A}, where each R^{7A} is independently H, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted 5- to 10-membered heteroaryl, or optionally substituted 4- to 10-membered heterocyclyl, or two geminal R^{7A} groups, together with the atom to which they are attached, combine to form optionally substituted 5- to 10-membered heteroaryl or optionally substituted 4- to 10-membered heterocyclyl.

In some embodiments, R⁵ and R⁶, together with the atoms to which they are attached, combine to form an optionally substituted 5- to 8-membered heterocyclyl. In some embodiments, R⁵ and R⁶, together with the atoms to which they are attached, combine to form an optionally substituted 7-membered heterocyclyl.

In some embodiments, R⁵ is optionally substituted C₁-C₆ alkyl. In some embodiments, R⁵ is optionally substituted amino. In some embodiments, R⁶ is optionally substituted C₁-C₆ alkyl. In some embodiments, R⁵ is halo.

In some embodiments, X¹ is SO₂. In some embodiments, X² is CR⁸.

In some embodiments,

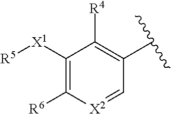

is a group of the following structure

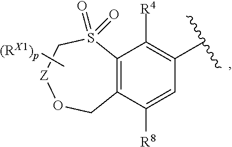

where
Z is CH₂, CO, or C(R^{X2})₂;
each R^{X1} is independently optionally substituted C₁-C₆ alkyl or halo, or two geminal R^{X1} groups, together with the atom to which they are attached, combine to form a carbonyl;
each R^{X1} is independently H or optionally substituted C₁-C₆ alkyl; and
p is 0, 1, 2, 3, or 4.

In some embodiments,

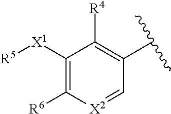

is a group of the following structure

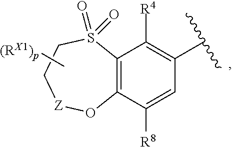

where
Z is CH₂, CO, or C(R^{X2})₂;
each R^{X1} is independently optionally substituted C₁-C₆ alkyl or halo, or two geminal R^{X1} groups, together with the atom to which they are attached, combine to form a carbonyl;
each R^{X2} is independently H or optionally substituted C₁-C₆ alkyl; and
p is 0, 1, 2, 3, or 4.

In some embodiments,

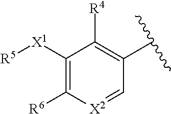

is a group of the following structure

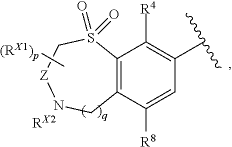

where
Z is CH₂, CO, or C(R^{X2})₂;
each R^{X1} is independently optionally substituted C₁-C₆ alkyl or halo, or two geminal R^{X1} groups, together with the atom to which they are attached, combine to form a carbonyl;
each R^{X2} is independently hydrogen or optionally substituted C₁-C₆ alkyl;
p is 0, 1, 2, 3, or 4; and
q is 0 or 1.

In some embodiments,

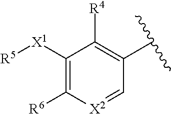

is a group of the following structure

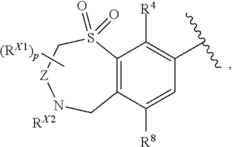

where
Z is CH₂, CO, or C(R^{X2})₂;
each R^{X1} is independently optionally substituted C₁-C₆ alkyl or halo, or two germinal R^{X1} groups, together with the atom to which they are attached, combine to form a carbonyl;
each R^{X2} is independently hydrogen or optionally substituted C₁-C₆ alkyl; and
p is 0, 1, 2, 3, or 4.

In some embodiments,

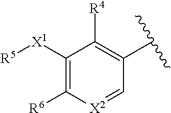

is a group of the following structure

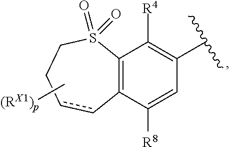

where
[symbol] is a single bond or double bond;
each R^{X1} is independently optionally substituted C₁-C₆ alkyl or halo, or two geminal R^{X1} groups, together with the atom to which they are attached, combine to form a carbonyl;
R^{X2} is hydrogen or optionally substituted C₁-C₆ alkyl; and
p is 0, 1, 2, 3, or 4.

In some embodiments,

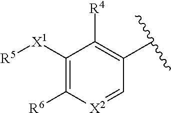

is a group of the following structure

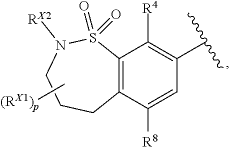

where
each R^{X1} is independently optionally substituted C₁-C₆ alkyl or halo, or two geminal R^{X1} groups, together with the atom to which they are attached, combine to form a carbonyl;
R^{X2} is hydrogen or optionally substituted C₁-C₆ alkyl; and
p is 0, 1, 2, 3, or 4.

In some embodiments, R⁸ is hydrogen.

In some embodiments, R⁸ is halo.

In some embodiments, R⁸ is optionally substituted C₃-C₈ cycloalkyl.

In some embodiments, X² is N.

In some embodiments,

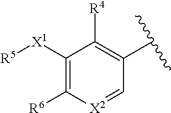

is a group of the following structure

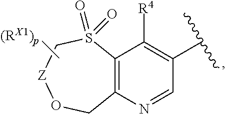

where
Z is CH₂, CO, or C(R^{X2})₂;
each R^{X1} is independently optionally substituted C₁-C₆ alkyl or halo, or two geminal R^{X1} groups, together with the atom to which they are attached, combine to form a carbonyl;
each R^{X2} is independently hydrogen or optionally substituted C₁-C₆ alkyl; and
p is 0, 1, 2, 3, or 4.

In some embodiments,

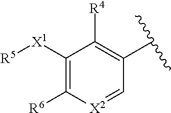

is a group of the following structure

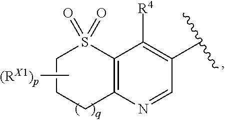

where
each R^{X1} is independently optionally substituted C₁-C₆ alkyl or halo, or two geminal R^{X1} groups, together with the atom to which they are attached, combine to form a carbonyl or C₃-C₈ cycloalkyl ring, or two vicinal R^{X1} groups, together with the atoms to which they are attached, combine to form C₃-C₈ cycloalkyl ring;
p is 0, 1, 2, 3, or 4; and
g is 0, 1, or 2.

In some embodiments,

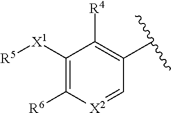

is a group of the following structure

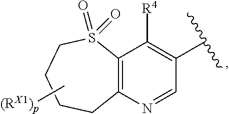

where
each R^{X1} is independently optionally substituted C₁-C₆ alkyl or halo, or two geminal R^{X1} groups, together with the atom to which they are attached, combine to form a carbonyl; and
p is 0, 1, 2, 3, or 4.

In some embodiments, at least one R^{X1} is optionally substituted C₁-C₆ alkyl.

In some embodiments, -L²-(R⁷)ₙ is a group of the following structure:

In some embodiments, at least one R^{X1} is halo.

In some embodiments, at least two geminal R^{X1} groups, together with the atom to which they are attached, combine to form a carbonyl.

In some embodiments, L¹ is optionally substituted 9- or 10-membered bicyclic heteroaryl.

In some embodiments, L¹ is

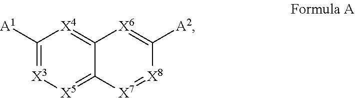

where
each of X³, X⁴, X⁵, X⁶, X⁷, and X⁸ is independently N or CR^{L1};
each R^{L1} is independently H, halo, optionally substituted C₁-C₆ alkyl;
A¹ is a bond to —(C(R²)(R³))ₘ—; and
A² is a bond to L².

In some embodiments, L¹ is

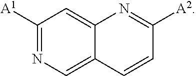

In some embodiments, L¹ is

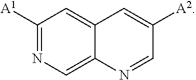

In some embodiments, L¹ is

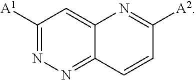

In some embodiments, L¹ is

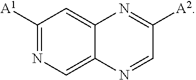

In some embodiments, L¹ is

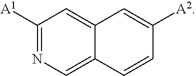

In some embodiments, L¹ is

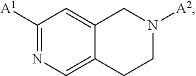

where
A¹ is a bond to —(C(R²)(R³))ₘ—; and
A² is a bond to L².

In some embodiments, L² is optionally substituted 5- to 10-membered heteroaryl.

In some embodiments, -L²-(R⁷)ₙ is a group of the following structure:

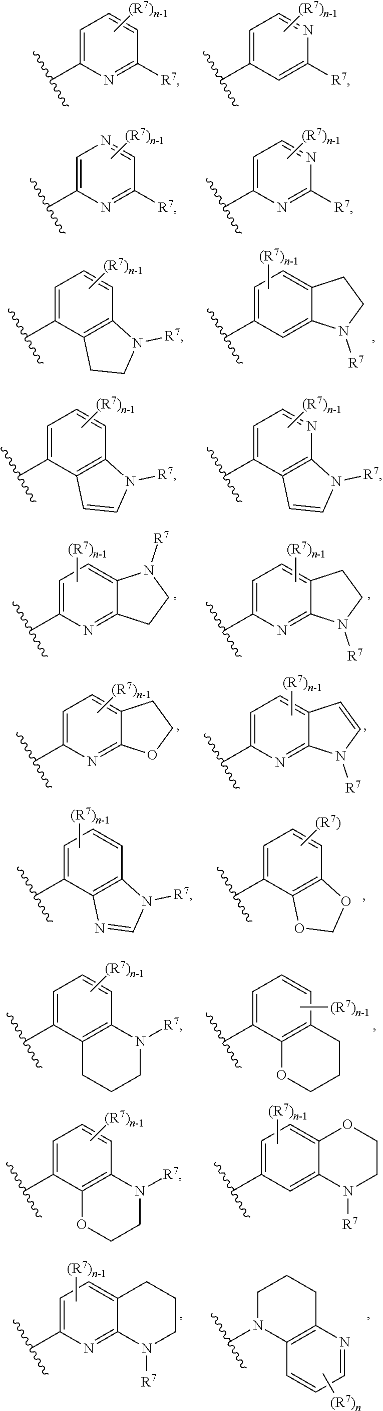

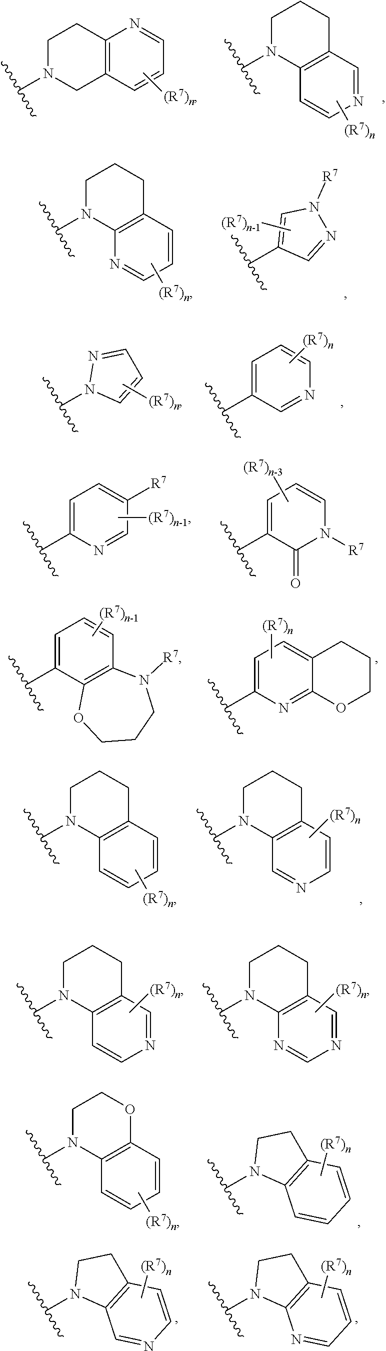

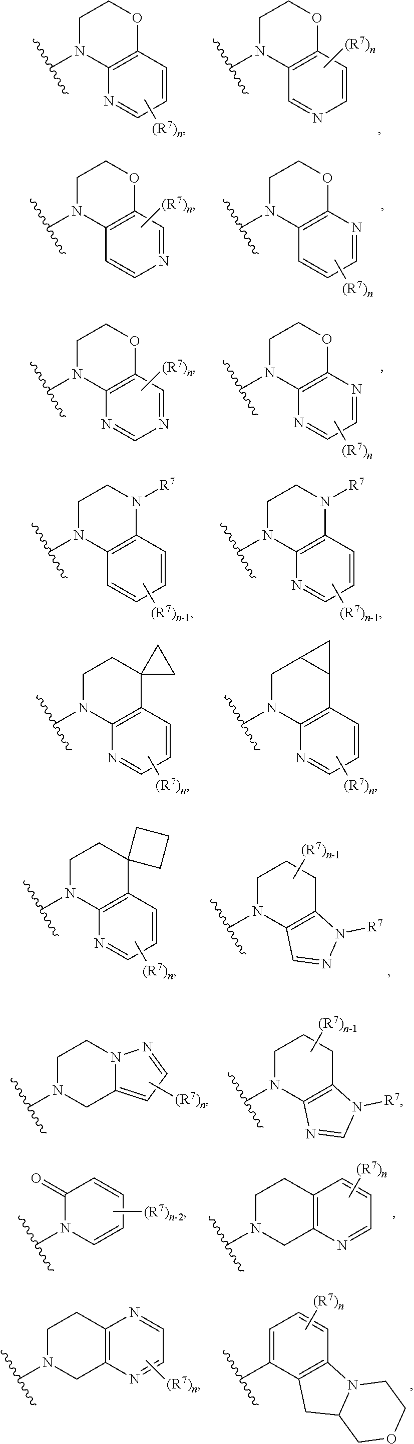

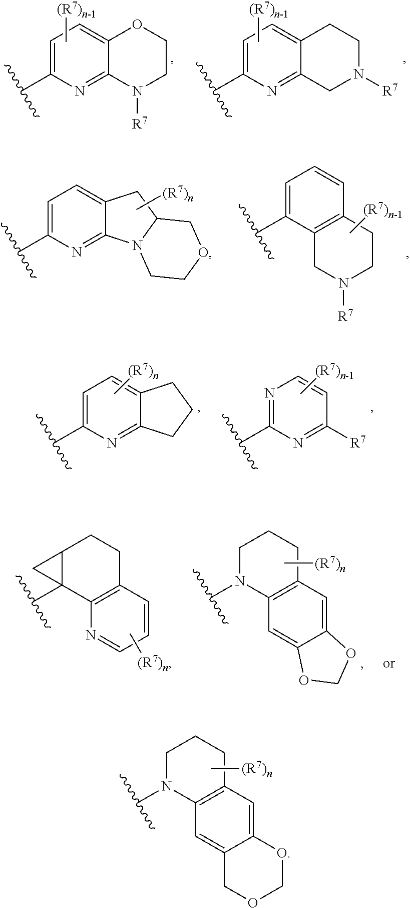

In some embodiments, -L²-(R⁷)ₙ is a group of the following structure:

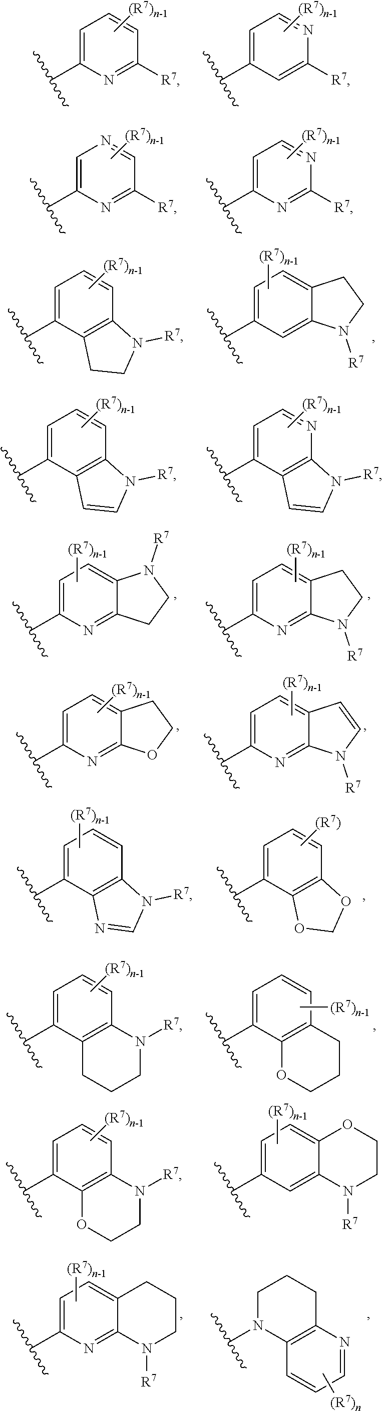

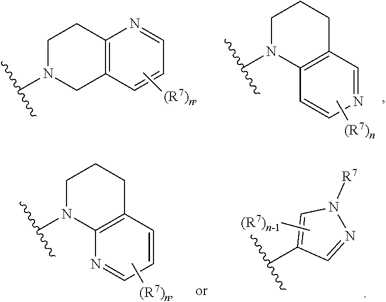

In some embodiments, -L²-(R⁷)ₙ is a group of the following structure:

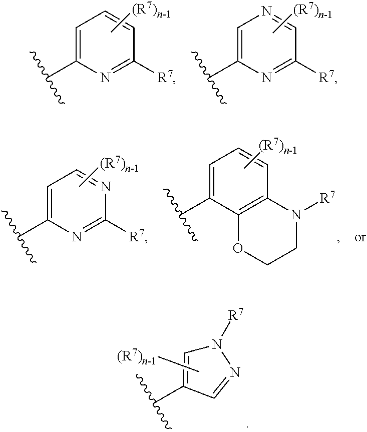

In some embodiments, -L²-(R⁷)ₙ is a group of the following structure:

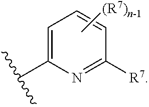

In some embodiments, -L²-(R⁷)ₙ is a group of the following structure:

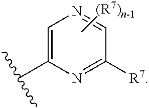

In some embodiments, -L²-(R⁷)ₙ is a group of the following structure:

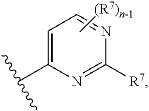

In some embodiments, -L²-(R⁷)ₙ is a group of the following structure:

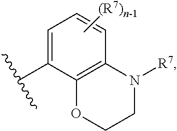

In some embodiments, -L²-(R⁷)ₙ is a group of the following structure:

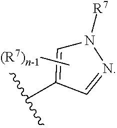

In some embodiments, -L²-(R⁷)ₙ is a group of the following structure:

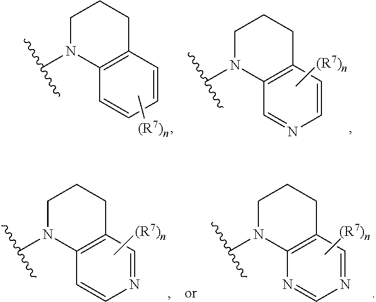

In some embodiments, -L²-(R⁷)ₙ is a group of the following structure:

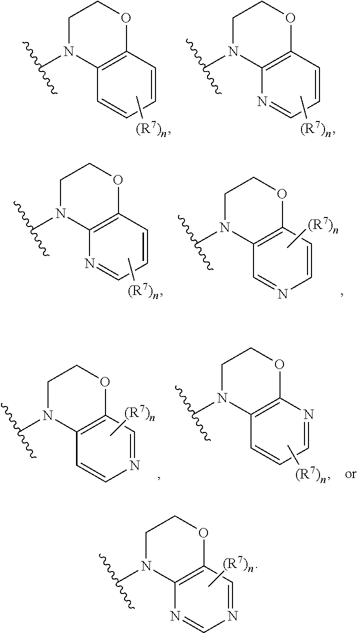

In some embodiments, L² is optionally substituted C₆-C₁₀ aryl.

In some embodiments, n is 1. In some embodiments, n is 2. In some embodiments, n is 3.

In some embodiments, R⁷ is optionally substituted C₁-C₆ alkyl. In some embodiments, R⁷ is optionally substituted C₁-C₆ heteroalkyl. In some embodiments, R⁷ is optionally substituted 4- to 10-membered heterocyclyl. In some embodiments, R⁷ is optionally substituted azetidinyl or optionally substituted morpholinyl. In some embodiments, R⁷ is optionally substituted C₃-C₁₀ cycloalkyl. In some embodiments, R⁷ is optionally substituted cyclopropyl or optionally substituted cyclobutyl. In some embodiments, R⁷ is —N(R^{7A})₂. In some embodiments, R⁷ is optionally substituted N-azetidinyl or optionally substituted N-morpholinyl. In some embodiments, two geminal R¹ groups, together with the atom to which they are attached, combine to form optionally substituted 4- to 10-membered heterocyclyl. In some embodiments, at least one R⁷ is —OR^{7A}. In some embodiments, R^{7A} is optionally substituted C₁₋₆ alkyl.

In some embodiments, n is 0.

In some embodiments, at least one R⁷ is cyclopropyl, 2,2-difluorocyclopropyl, difluoromethoxy, 2,6-dimethylmorpholin-4-yl, N-azetidinyl, 3-fluorocyclobutyl, 2-methoxyethyl, ethoxy, methoxy, 2,2-difluoroethoxy, 2,2-difluoroethyl, trifluoromethyl, isopropyl, methyl, acetyl, fluoro, chloro, 1-methylpyrazol-3-yl, dimethylamino, N-methyl-N-(2-methoxyethyl)-amino, N-ethyl-N-(2-methoxyethyl)-amino, N-(2-propyl)-N-(2-methoxyethyl)-amino, 2-methoxyethylamino, 3-aza-8-oxa-bicyclo[4.3.0]non-3-yl, 3-aza-7-oxa-bicyclo[4.3.0]non-3-yl, 1-fluorocyclobut-1-yl, 3-fluoropyrrolidin-1-yl, 3-methoxypyrrolidin-1-yl, oxetan-3-yl, N-methylindolin-4-yl, 2,2-difluoro-3-methylcycloprop-1-yl, 3-methoxyazetidin-1-yl, 3-methoxypiperidin-1-yl, 1,2-dimethyl-7-azaindol-4-yl, 1-methyl-7-azaindol-4-yl, 2,3-methylenedioxyphenyl, N-methyl-N-(3-oxetanyl)amino, 3-oxetanyloxy, 1,1-difluoro-5-azaspiro[2.3]hex-5-yl, 1-fluoromethyl-cyclopropyl, N-(3-tetrahydrofuranyl)methylamino, N-indolinyl, N-1,4-oxazepanyl, 2-fluoro-2-propyl, 1,1-difluoro-2-propyl, 2,2-difluoro-1-methylcycloprop-1-yl, 1-methylcyclopropyl, 4,4-difluoropiperidin-1-yl, 2-methoxyethoxy, 3,3-difluorocyclobut-1-yl, N-methyl-N-1-methoxyprop-2-ylamino, 1-methoxyprop-2-ylamino, 1-methoxyethyl, 4-methylpiperazinyl, 3-methylmorpholinyl, 2,2-difluoropropoxy, 3-methoxycyclobutyl, methylamino, 4-dimethylamino-3,3-difluoropiperidinyl, 4-methylamino-3,3-difluoropiperidinyl, 3,3-difluoropyrrolidinyl, N-methyl-N-3-methoxycyclobutylamino, 1-methylpyrazol-5-yl, 6-oxa-3-azabicyclo[3.1.1]hept-3-yl, cyclopropyloxy, 2,6-dimethylpyrid-4-yl, 2-methylpyrrolldinyl, 4-oxabicyclo[4.1.0]hept-1-yl, N-methyl-N-(2,6-dimethyltetrahydropyran-4-yl)amino, or N-methyl-N-3-methyloxetan-3-ylmethylamino.

In some embodiments, R¹ is hydrogen.

In another aspect, the invention provides a compound selected from the group consisting of compounds 1-308 in Table 1A and pharmaceutically acceptable salts thereof.

TABLE 1A
Compounds of the invention

| Compound | |
|---|---|
| # | Structure |
| 1 | 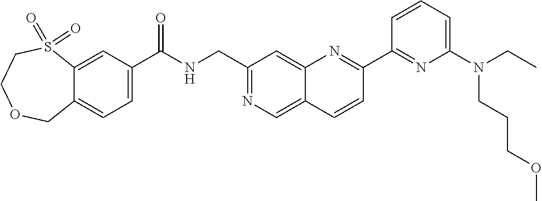 [SMILES: CCN(CCCOC)c1cccc(-c2ccc3cnc(CNC(=O)c4ccc5c(c4)S(=O)(=O)CCOC5)cc3n2)n1] |
| 2 | 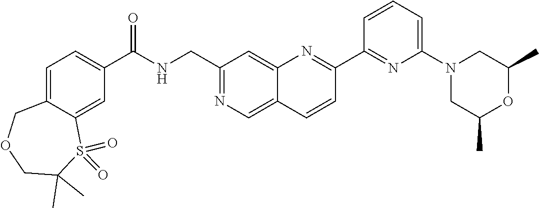 [SMILES: C[C@@H]1CN(c2cccc(-c3ccc4cnc(CNC(=O)c5ccc6c(c5)S(=O)(=O)C(C)(C)COC6)cc4n3)n2)C[C@H](C)O1] |
| 3 | 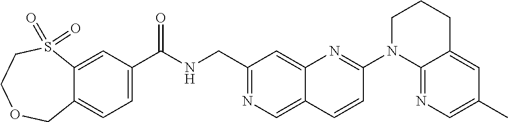 [SMILES: Cc1cnc2c(c1)CCCN2c1ccc2cnc(CNC(=O)c3ccc4c(c3)S(=O)(=O)CCOC4)cc2n1] |
| 4 | 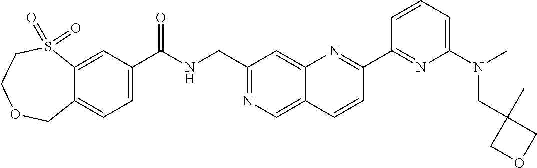 [SMILES: CN(CC1(C)COC1)c1cccc(-c2ccc3cnc(CNC(=O)c4ccc5c(c4)S(=O)(=O)CCOC5)cc3n2)n1] |
| 5 | 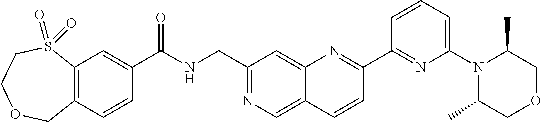 [SMILES: C[C@H]1COC[C@H](C)N1c1cccc(-c2ccc3cnc(CNC(=O)c4ccc5c(c4)S(=O)(=O)CCOC5)cc3n2)n1] |
| 6 | 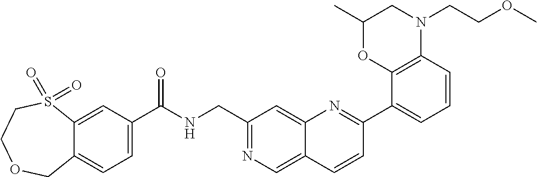 [SMILES: COCCN1CC(C)Oc2c(-c3ccc4cnc(CNC(=O)c5ccc6c(c5)S(=O)(=O)CCOC6)cc4n3)cccc21] |
| 7 | 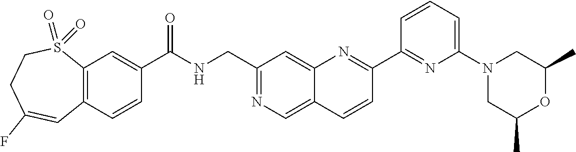 [SMILES: C[C@@H]1CN(c2cccc(-c3ccc4cnc(CNC(=O)c5ccc6c(c5)S(=O)(=O)CCC(F)=C6)cc4n3)n2)C[C@H](C)O1] |
| 8 | 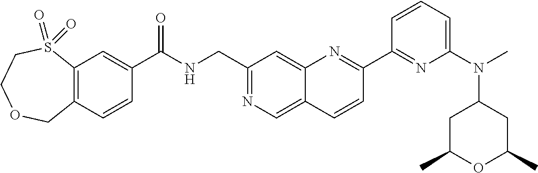 [SMILES: C[C@@H]1CC(N(C)c2cccc(-c3ccc4cnc(CNC(=O)c5ccc6c(c5)S(=O)(=O)CCOC6)cc4n3)n2)C[C@H](C)O1] |
| 9 | 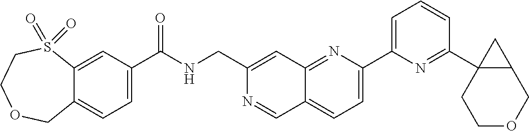 [SMILES: O=C(NCc1cc2nc(-c3cccc(C45CCOCC4C5)n3)ccc2cn1)c1ccc2c(c1)S(=O)(=O)CCOC2] |
| 10 | 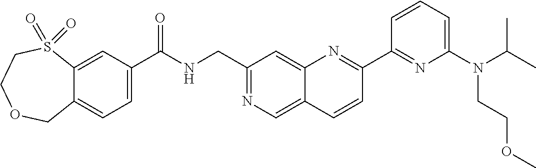 [SMILES: COCCN(c1cccc(-c2ccc3cnc(CNC(=O)c4ccc5c(c4)S(=O)(=O)CCOC5)cc3n2)n1)C(C)C] |
| 11 | 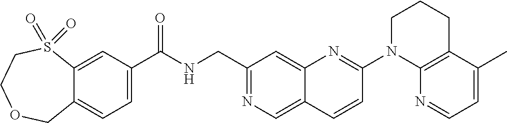 [SMILES: Cc1ccnc2c1CCCN2c1ccc2cnc(CNC(=O)c3ccc4c(c3)S(=O)(=O)CCOC4)cc2n1] |
| 12 | 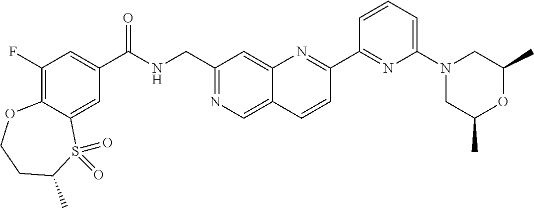 [SMILES: C[C@@H]1CN(c2cccc(-c3ccc4cnc(CNC(=O)c5cc(F)c6c(c5)S(=O)(=O)[C@H](C)CCO6)cc4n3)n2)C[C@H](C)O1] |
| 13 | 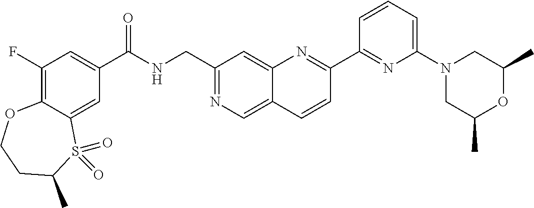 [SMILES: C[C@@H]1CN(c2cccc(-c3ccc4cnc(CNC(=O)c5cc(F)c6c(c5)S(=O)(=O)[C@@H](C)CCO6)cc4n3)n2)C[C@H](C)O1] |
| 14 | 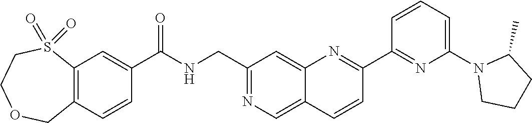 [SMILES: C[C@@H]1CCCN1c1cccc(-c2ccc3cnc(CNC(=O)c4ccc5c(c4)S(=O)(=O)CCOC5)cc3n2)n1] |
| 15 | 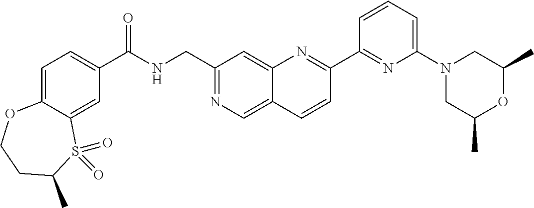 [SMILES: C[C@@H]1CN(c2cccc(-c3ccc4cnc(CNC(=O)c5ccc6c(c5)S(=O)(=O)[C@@H](C)CCO6)cc4n3)n2)C[C@H](C)O1] |
| 16 | 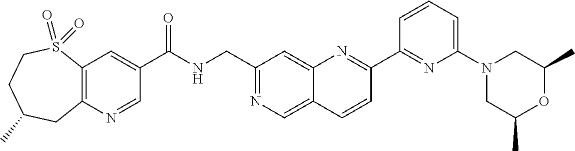 |
| 17 | 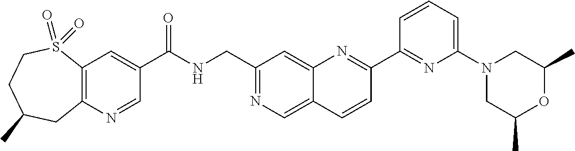 |
| 18 | 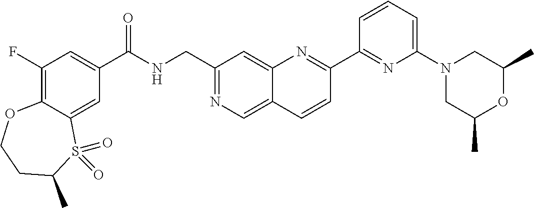 [SMILES: C[C@@H]1CN(c2cccc(-c3ccc4cnc(CNC(=O)c5cc(F)c6c(c5)S(=O)(=O)[C@@H](C)CCO6)cc4n3)n2)C[C@H](C)O1] |
| 19 | 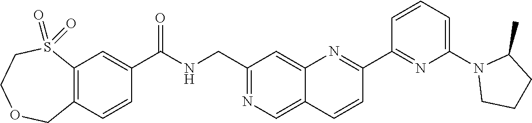 [SMILES: C[C@H]1CCCN1c1cccc(-c2ccc3cnc(CNC(=O)c4ccc5c(c4)S(=O)(=O)CCOC5)cc3n2)n1] |
| 20 | 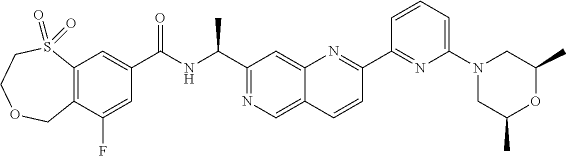 [SMILES: C[C@@H]1CN(c2cccc(-c3ccc4cnc([C@H](C)NC(=O)c5cc(F)c6c(c5)S(=O)(=O)CCOC6)cc4n3)n2)C[C@H](C)O1] |
| 21 | 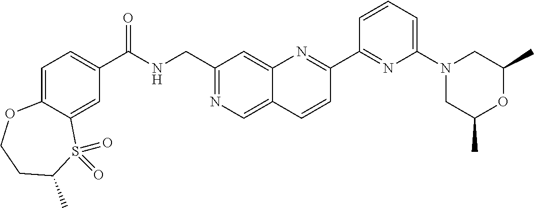 [SMILES: C[C@@H]1CN(c2cccc(-c3ccc4cnc(CNC(=O)c5ccc6c(c5)S(=O)(=O)[C@H](C)CCO6)cc4n3)n2)C[C@H](C)O1] |
| 22 | 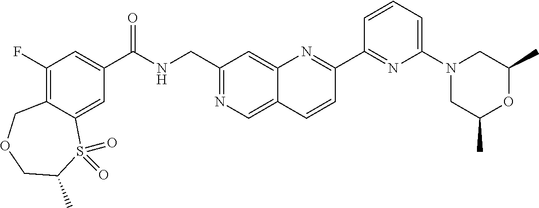 [SMILES: C[C@@H]1CN(c2cccc(-c3ccc4cnc(CNC(=O)c5cc(F)c6c(c5)S(=O)(=O)[C@H](C)COC6)cc4n3)n2)C[C@H](C)O1] |
| 23 | 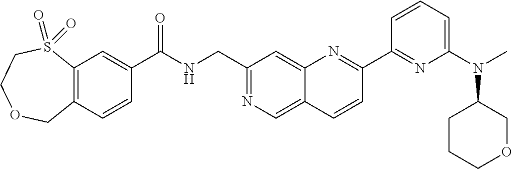 [SMILES: CN(c1cccc(-c2ccc3cnc(CNC(=O)c4ccc5c(c4)S(=O)(=O)CCOC5)cc3n2)n1)[C@@H]1CCCOC1] |
| 24 | 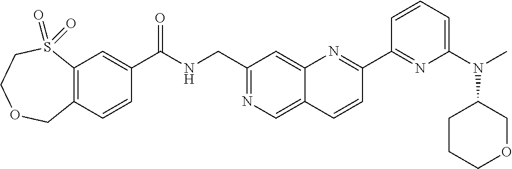 [SMILES: CN(c1cccc(-c2ccc3cnc(CNC(=O)c4ccc5c(c4)S(=O)(=O)CCOC5)cc3n2)n1)[C@H]1CCCOC1] |
| 25 | 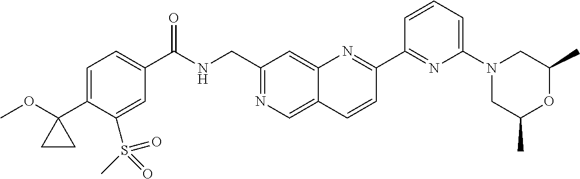 |
| 26 | 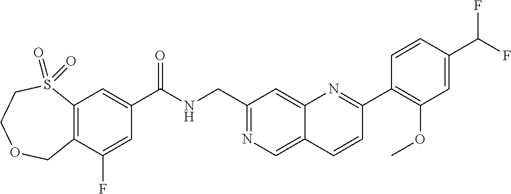 [SMILES: COc1cc(C(F)F)ccc1-c1ccc2cnc(CNC(=O)c3cc(F)c4c(c3)S(=O)(=O)CCOC4)cc2n1] |
| 27 | 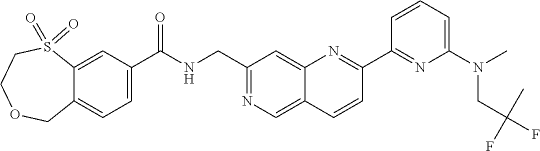 [SMILES: CN(CC(C)(F)F)c1cccc(-c2ccc3cnc(CNC(=O)c4ccc5c(c4)S(=O)(=O)CCOC5)cc3n2)n1] |
| 28 | 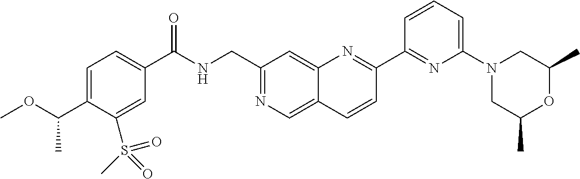 |
| 29 | 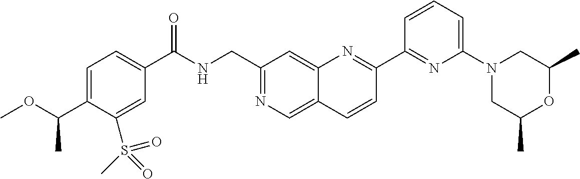 |
| 30 | 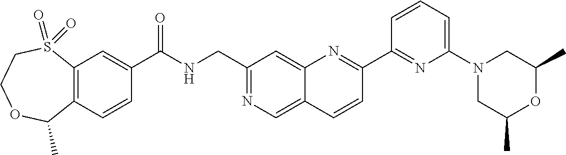 [SMILES: C[C@@H]1CN(c2cccc(-c3ccc4cnc(CNC(=O)c5ccc6c(c5)S(=O)(=O)CCO[C@H]6C)cc4n3)n2)C[C@H](C)O1] |
| 31 | 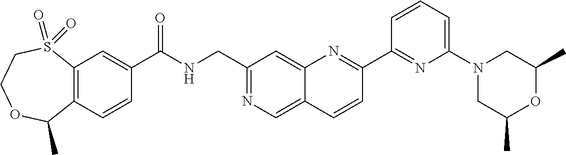 [SMILES: C[C@@H]1CN(c2cccc(-c3ccc4cnc(CNC(=O)c5ccc6c(c5)S(=O)(=O)CCO[C@@H]6C)cc4n3)n2)C[C@H](C)O1] |
| 32 | 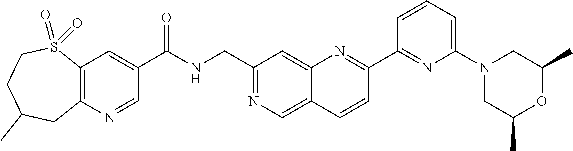 |
| 33 | 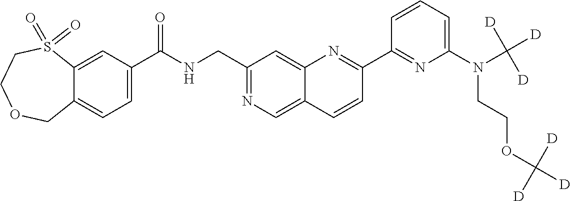 [SMILES: [2H]C([2H])([2H])OCCN(c1cccc(-c2ccc3cnc(CNC(=O)c4ccc5c(c4)S(=O)(=O)CCOC5)cc3n2)n1)C([2H])([2H])[2H]] |
| 34 | 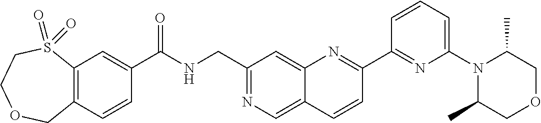 [SMILES: C[C@@H]1COC[C@@H](C)N1c1cccc(-c2ccc3cnc(CNC(=O)c4ccc5c(c4)S(=O)(=O)CCOC5)cc3n2)n1] |
| 35 | 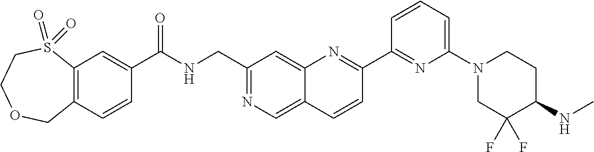 [SMILES: CN[C@@H]1CCN(c2cccc(-c3ccc4cnc(CNC(=O)c5ccc6c(c5)S(=O)(=O)CCOC6)cc4n3)n2)CC1(F)F] |
| 36 | 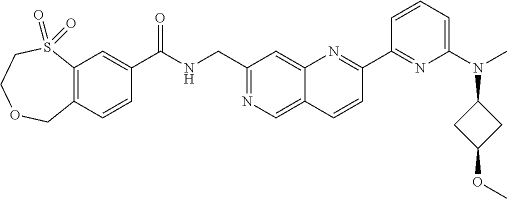 [SMILES: CO[C@H]1C[C@@H](N(C)c2cccc(-c3ccc4cnc(CNC(=O)c5ccc6c(c5)S(=O)(=O)CCOC6)cc4n3)n2)C1] |
| 37 | 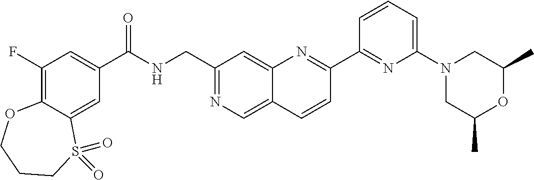 [SMILES: C[C@@H]1CN(c2cccc(-c3ccc4cnc(CNC(=O)c5cc(F)c6c(c5)S(=O)(=O)CCCO6)cc4n3)n2)C[C@H](C)O1] |
| 38 | 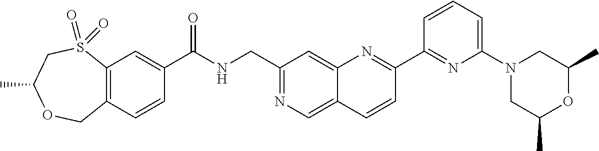 |
| 39 | 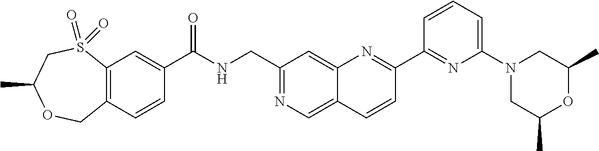 |
| 40 | 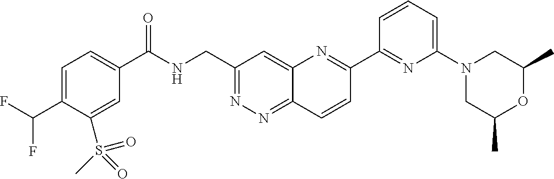 [SMILES: C[C@@H]1CN(c2cccc(-c3ccc4nnc(CNC(=O)c5ccc(C(F)F)c(S(C)(=O)=O)c5)cc4n3)n2)C[C@H](C)O1] |
| 41 | 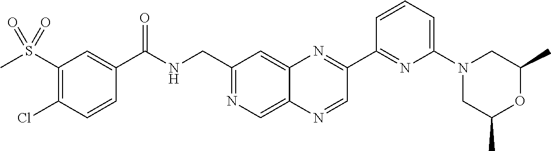 |
| 42 | 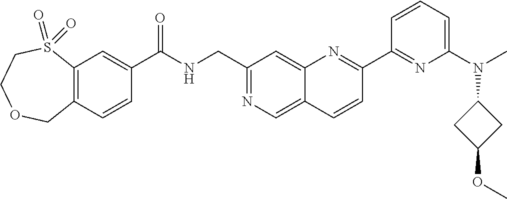 [SMILES: CO[C@H]1C[C@H](N(C)c2cccc(-c3ccc4cnc(CNC(=O)c5ccc6c(c5)S(=O)(=O)CCOC6)cc4n3)n2)C1] |
| 43 | 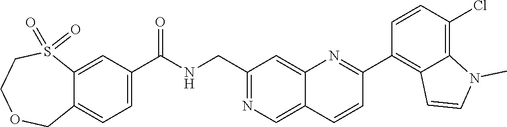 [SMILES: Cn1ccc2c(-c3ccc4cnc(CNC(=O)c5ccc6c(c5)S(=O)(=O)CCOC6)cc4n3)ccc(Cl)c21] |
| 44 | 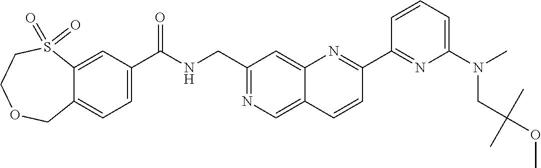 [SMILES: COC(C)(C)CN(C)c1cccc(-c2ccc3cnc(CNC(=O)c4ccc5c(c4)S(=O)(=O)CCOC5)cc3n2)n1] |
| 45 | 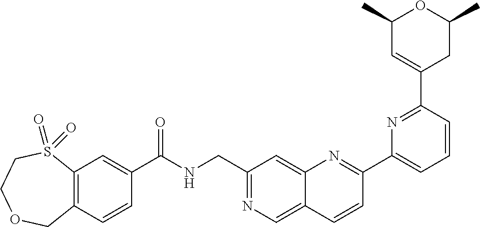 [SMILES: C[C@@H]1C=C(c2cccc(-c3ccc4cnc(CNC(=O)c5ccc6c(c5)S(=O)(=O)CCOC6)cc4n3)n2)C[C@H](C)O1] |
| 46 | 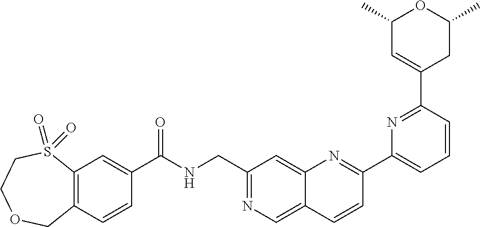 [SMILES: C[C@@H]1CC(c2cccc(-c3ccc4cnc(CNC(=O)c5ccc6c(c5)S(=O)(=O)CCOC6)cc4n3)n2)=C[C@H](C)O1] |
| 47 | 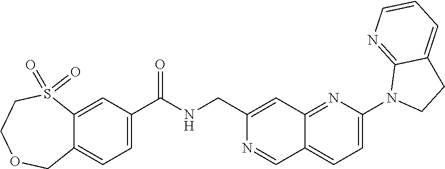 [SMILES: O=C(NCc1cc2nc(N3CCc4cccnc43)ccc2cn1)c1ccc2c(c1)S(=O)(=O)CCOC2] |
| 48 | 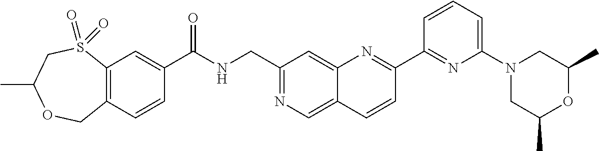 |
| 49 | 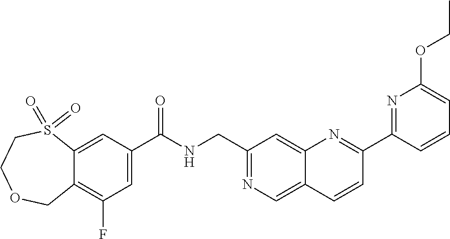 [SMILES: CCOc1cccc(-c2ccc3cnc(CNC(=O)c4cc(F)c5c(c4)S(=O)(=O)CCOC5)cc3n2)n1] |
| 50 | 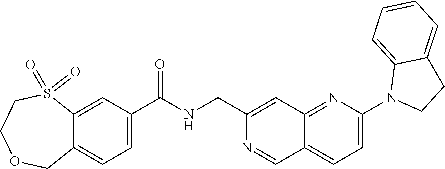 [SMILES: O=C(NCc1cc2nc(N3CCc4ccccc43)ccc2cn1)c1ccc2c(c1)S(=O)(=O)CCOC2] |
| 51 | 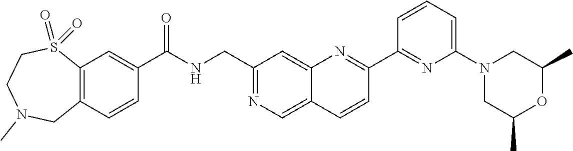 |
| 52 | 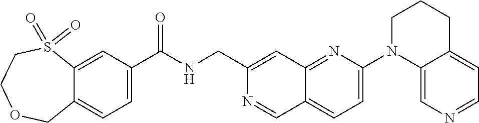 [SMILES: O=C(NCc1cc2nc(N3CCCc4ccncc43)ccc2cn1)c1ccc2c(c1)S(=O)(=O)CCOC2] |
| 53 | 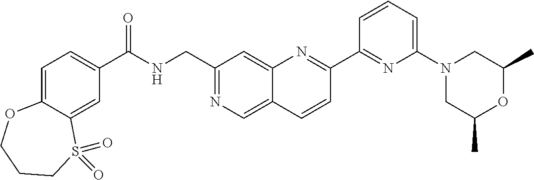 [SMILES: C[C@@H]1CN(c2cccc(-c3ccc4cnc(CNC(=O)c5ccc6c(c5)S(=O)(=O)CCCO6)cc4n3)n2)C[C@H](C)O1] |
| 54 | 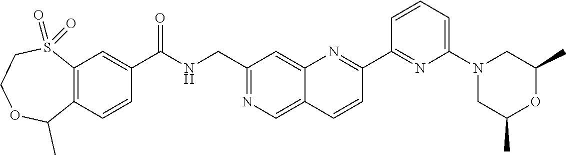 [SMILES: CC1OCCS(=O)(=O)c2cc(C(=O)NCc3cc4nc(-c5cccc(N6C[C@@H](C)O[C@@H](C)C6)n5)ccc4cn3)ccc21] |
| 55 | 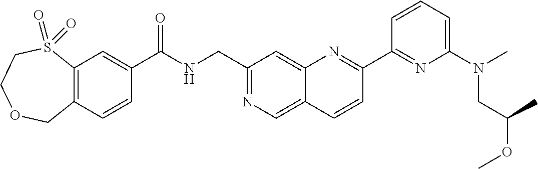 [SMILES: CO[C@H](C)CN(C)c1cccc(-c2ccc3cnc(CNC(=O)c4ccc5c(c4)S(=O)(=O)CCOC5)cc3n2)n1] |
| 56 | 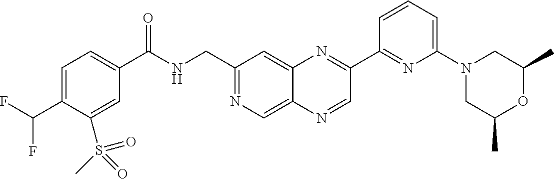 [SMILES: C[C@@H]1CN(c2cccc(-c3cnc4cnc(CNC(=O)c5ccc(C(F)F)c(S(C)(=O)=O)c5)cc4n3)n2)C[C@H](C)O1] |
| 57 | 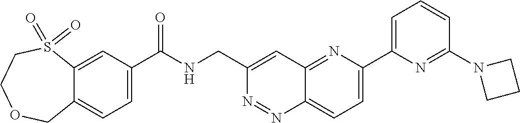 [SMILES: O=C(NCc1cc2nc(-c3cccc(N4CCC4)n3)ccc2nn1)c1ccc2c(c1)S(=O)(=O)CCOC2] |
| 58 | 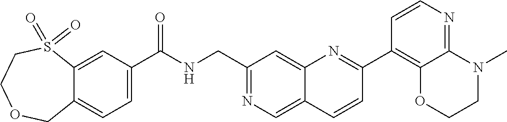 [SMILES: CN1CCOc2c(-c3ccc4cnc(CNC(=O)c5ccc6c(c5)S(=O)(=O)CCOC6)cc4n3)ccnc21] |
| 59 | 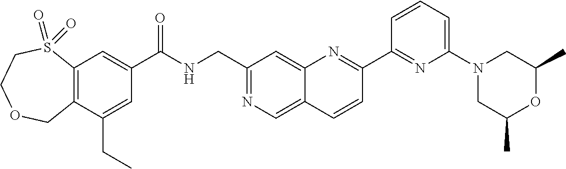 [SMILES: CCc1cc(C(=O)NCc2cc3nc(-c4cccc(N5C[C@@H](C)O[C@@H](C)C5)n4)ccc3cn2)cc2c1COCCS2(=O)=O] |
| 60 | 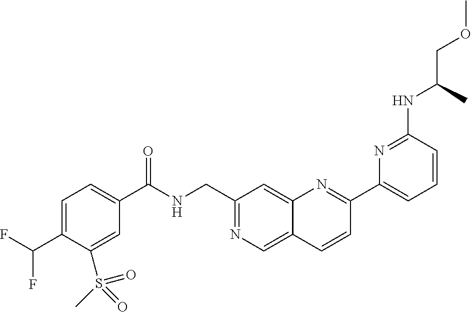 [SMILES: COC[C@@H](C)Nc1cccc(-c2ccc3cnc(CNC(=O)c4ccc(C(F)F)c(S(C)(=O)=O)c4)cc3n2)n1] |
| 61 | 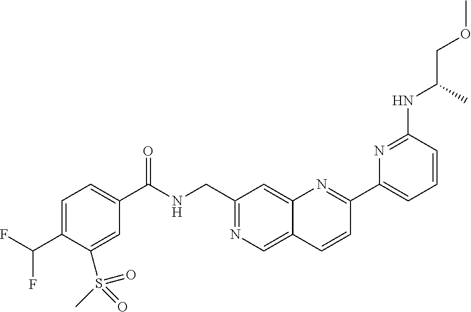 [SMILES: COC[C@H](C)Nc1cccc(-c2ccc3cnc(CNC(=O)c4ccc(C(F)F)c(S(C)(=O)=O)c4)cc3n2)n1] |
| 62 | 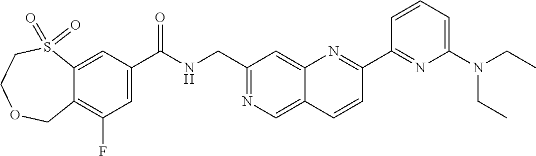 [SMILES: CCN(CC)c1cccc(-c2ccc3cnc(CNC(=O)c4cc(F)c5c(c4)S(=O)(=O)CCOC5)cc3n2)n1] |
| 63 | 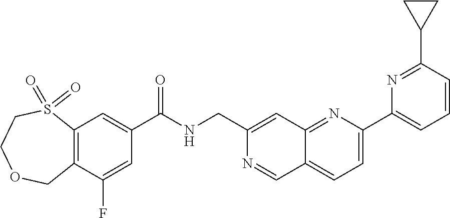 [SMILES: O=C(NCc1cc2nc(-c3cccc(C4CC4)n3)ccc2cn1)c1cc(F)c2c(c1)S(=O)(=O)CCOC2] |
| 64 | 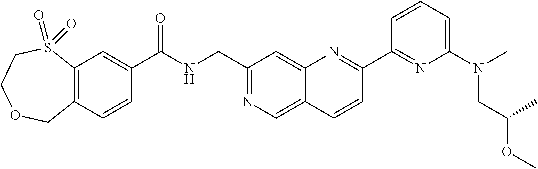 [SMILES: CO[C@@H](C)CN(C)c1cccc(-c2ccc3cnc(CNC(=O)c4ccc5c(c4)S(=O)(=O)CCOC5)cc3n2)n1] |
| 65 | 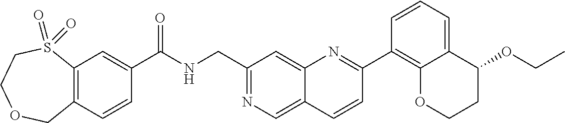 [SMILES: CCO[C@@H]1CCOc2c(-c3ccc4cnc(CNC(=O)c5ccc6c(c5)S(=O)(=O)CCOC6)cc4n3)cccc21] |
| 66 | 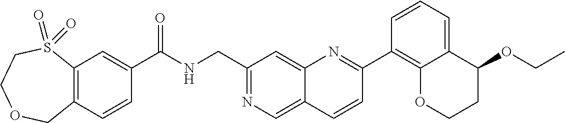 [SMILES: CCO[C@H]1CCOc2c(-c3ccc4cnc(CNC(=O)c5ccc6c(c5)S(=O)(=O)CCOC6)cc4n3)cccc21] |
| 67 | 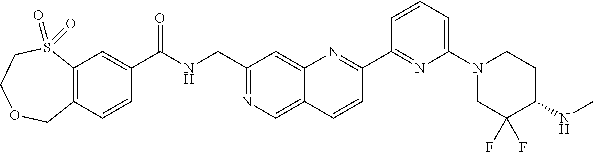 [SMILES: CN[C@H]1CCN(c2cccc(-c3ccc4cnc(CNC(=O)c5ccc6c(c5)S(=O)(=O)CCOC6)cc4n3)n2)CC1(F)F] |
| 68 | 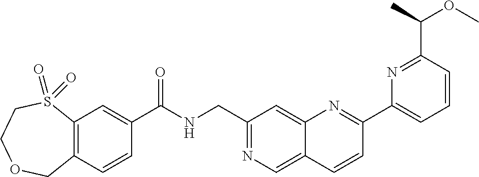 [SMILES: CO[C@H](C)c1cccc(-c2ccc3cnc(CNC(=O)c4ccc5c(c4)S(=O)(=O)CCOC5)cc3n2)n1] |
| 69 | 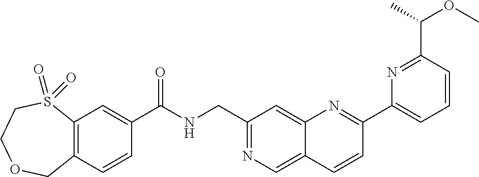 [SMILES: CO[C@@H](C)c1cccc(-c2ccc3cnc(CNC(=O)c4ccc5c(c4)S(=O)(=O)CCOC5)cc3n2)n1] |
| 70 | 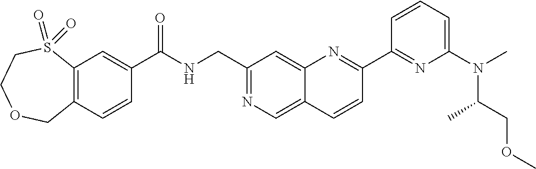 [SMILES: COC[C@H](C)N(C)c1cccc(-c2ccc3cnc(CNC(=O)c4ccc5c(c4)S(=O)(=O)CCOC5)cc3n2)n1] |
| 71 | 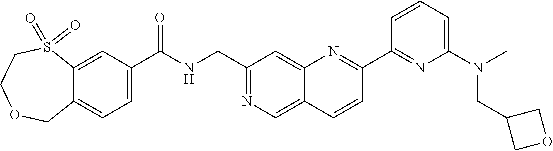 [SMILES: CN(CC1COC1)c1cccc(-c2ccc3cnc(CNC(=O)c4ccc5c(c4)S(=O)(=O)CCOC5)cc3n2)n1] |
| 72 | 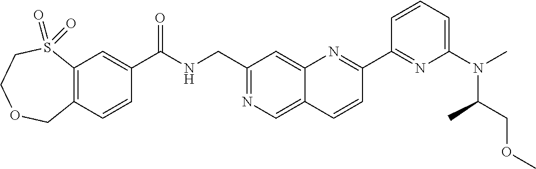 [SMILES: COC[C@@H](C)N(C)c1cccc(-c2ccc3cnc(CNC(=O)c4ccc5c(c4)S(=O)(=O)CCOC5)cc3n2)n1] |
| 73 | 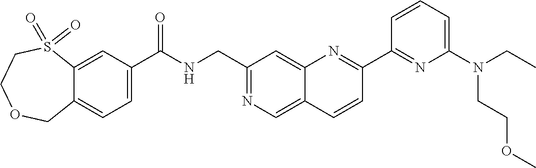 [SMILES: CCN(CCOC)c1cccc(-c2ccc3cnc(CNC(=O)c4ccc5c(c4)S(=O)(=O)CCOC5)cc3n2)n1] |
| 74 | 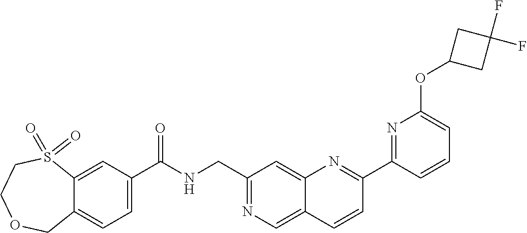 [SMILES: O=C(NCc1cc2nc(-c3cccc(OC4CC(F)(F)C4)n3)ccc2cn1)c1ccc2c(c1)S(=O)(=O)CCOC2] |
| 75 | 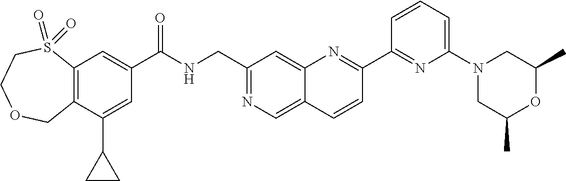 [SMILES: C[C@@H]1CN(c2cccc(-c3ccc4cnc(CNC(=O)c5cc(C6CC6)c6c(c5)S(=O)(=O)CCOC6)cc4n3)n2)C[C@H](C)O1] |
| 76 | 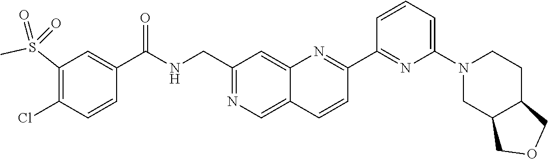 [SMILES: CS(=O)(=O)c1cc(C(=O)NCc2cc3nc(-c4cccc(N5CC[C@H]6COC[C@H]6C5)n4)ccc3cn2)ccc1Cl] |
| 77 | 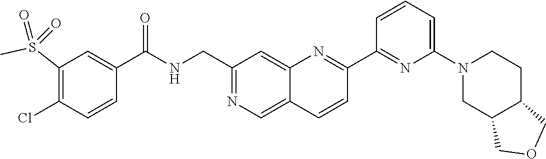 [SMILES: CS(=O)(=O)c1cc(C(=O)NCc2cc3nc(-c4cccc(N5CC[C@@H]6COC[C@@H]6C5)n4)ccc3cn2)ccc1Cl] |
| 78 | 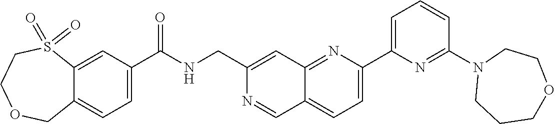 [SMILES: O=C(NCc1cc2nc(-c3cccc(N4CCCOCC4)n3)ccc2cn1)c1ccc2c(c1)S(=O)(=O)CCOC2] |
| 79 | 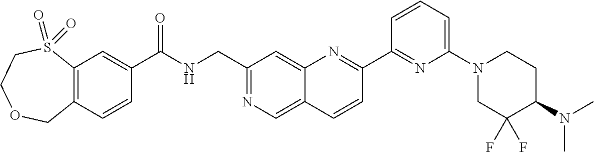 [SMILES: CN(C)[C@@H]1CCN(c2cccc(-c3ccc4cnc(CNC(=O)c5ccc6c(c5)S(=O)(=O)CCOC6)cc4n3)n2)CC1(F)F] |
| 80 | 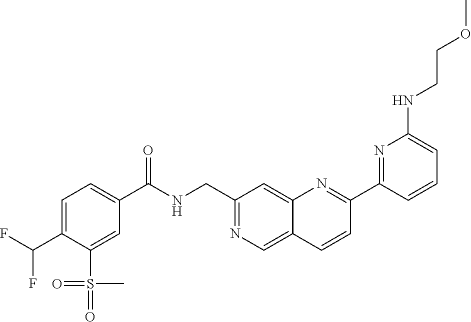 [SMILES: COCCNc1cccc(-c2ccc3cnc(CNC(=O)c4ccc(C(F)F)c(S(C)(=O)=O)c4)cc3n2)n1] |
| 81 | 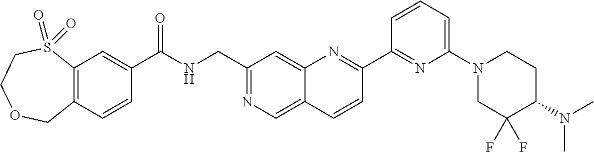 [SMILES: CN(C)[C@H]1CCN(c2cccc(-c3ccc4cnc(CNC(=O)c5ccc6c(c5)S(=O)(=O)CCOC6)cc4n3)n2)CC1(F)F] |
| 82 | 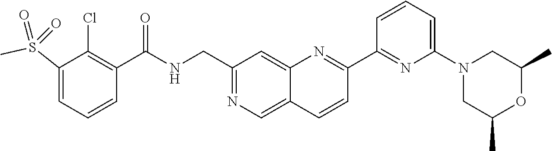 |
| 83 | 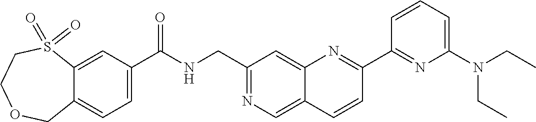 [SMILES: CCN(CC)c1cccc(-c2ccc3cnc(CNC(=O)c4ccc5c(c4)S(=O)(=O)CCOC5)cc3n2)n1] |
| 84 | 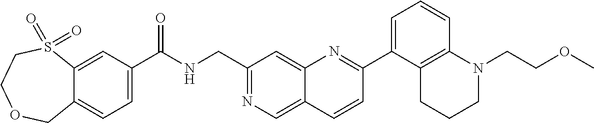 [SMILES: COCCN1CCCc2c(-c3ccc4cnc(CNC(=O)c5ccc6c(c5)S(=O)(=O)CCOC6)cc4n3)cccc21] |
| 85 | 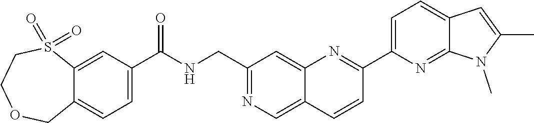 [SMILES: Cc1cc2ccc(-c3ccc4cnc(CNC(=O)c5ccc6c(c5)S(=O)(=O)CCOC6)cc4n3)nc2n1C] |
| 86 | 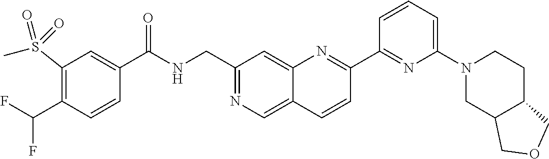 |
| 87 | 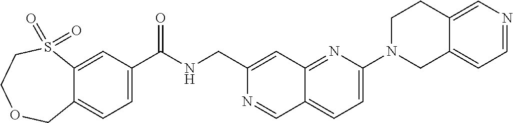 [SMILES: O=C(NCc1cc2nc(N3CCc4cnccc4C3)ccc2cn1)c1ccc2c(c1)S(=O)(=O)CCOC2] |
| 88 | 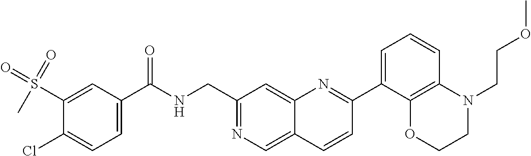 [SMILES: COCCN1CCOc2c(-c3ccc4cnc(CNC(=O)c5ccc(Cl)c(S(C)(=O)=O)c5)cc4n3)cccc21] |
| 89 | 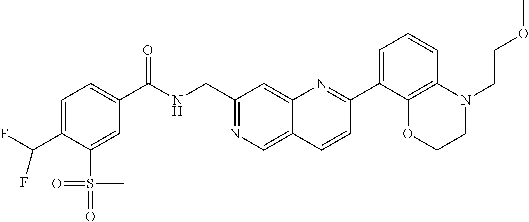 [SMILES: COCCN1CCOc2c(-c3ccc4cnc(CNC(=O)c5ccc(C(F)F)c(S(C)(=O)=O)c5)cc4n3)cccc21] |
| 90 | 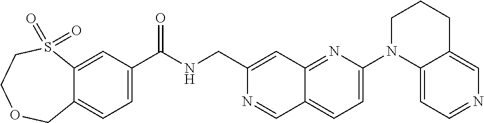 [SMILES: O=C(NCc1cc2nc(N3CCCc4cnccc43)ccc2cn1)c1ccc2c(c1)S(=O)(=O)CCOC2] |
| 91 | 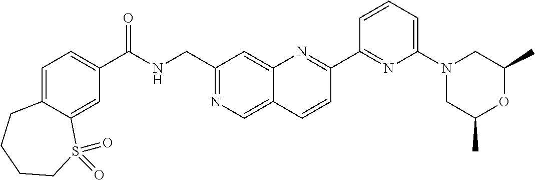 [SMILES: C[C@@H]1CN(c2cccc(-c3ccc4cnc(CNC(=O)c5ccc6c(c5)S(=O)(=O)CCCC6)cc4n3)n2)C[C@H](C)O1] |
| 92 | 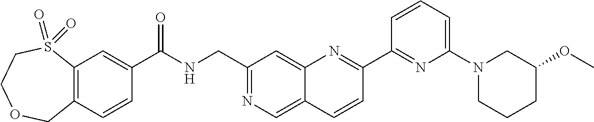 [SMILES: CO[C@@H]1CCCN(c2cccc(-c3ccc4cnc(CNC(=O)c5ccc6c(c5)S(=O)(=O)CCOC6)cc4n3)n2)C1] |
| 93 | 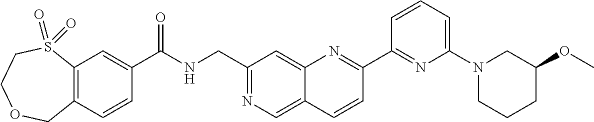 [SMILES: CO[C@H]1CCCN(c2cccc(-c3ccc4cnc(CNC(=O)c5ccc6c(c5)S(=O)(=O)CCOC6)cc4n3)n2)C1] |
| 94 | 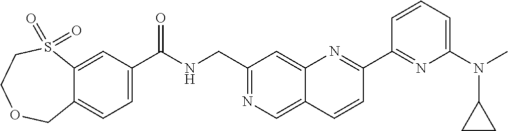 [SMILES: CN(c1cccc(-c2ccc3cnc(CNC(=O)c4ccc5c(c4)S(=O)(=O)CCOC5)cc3n2)n1)C1CC1] |
| 95 | 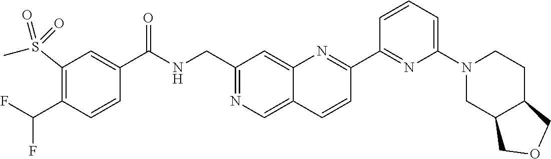 [SMILES: CS(=O)(=O)c1cc(C(=O)NCc2cc3nc(-c4cccc(N5CC[C@H]6COC[C@H]6C5)n4)ccc3cn2)ccc1C(F)F] |
| 96 | 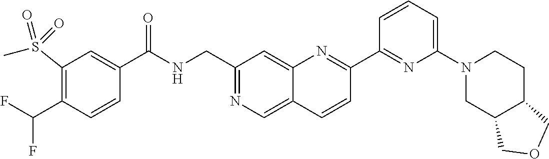 [SMILES: CS(=O)(=O)c1cc(C(=O)NCc2cc3nc(-c4cccc(N5CC[C@@H]6COC[C@@H]6C5)n4)ccc3cn2)ccc1C(F)F] |
| 97 | 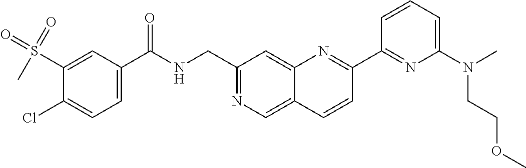 [SMILES: COCCN(C)c1cccc(-c2ccc3cnc(CNC(=O)c4ccc(Cl)c(S(C)(=O)=O)c4)cc3n2)n1] |
| 98 | 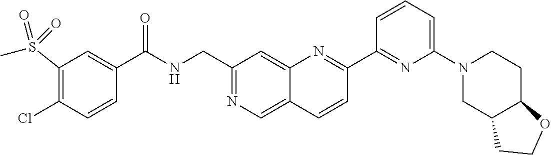 [SMILES: CS(=O)(=O)c1cc(C(=O)NCc2cc3nc(-c4cccc(N5CC[C@H]6OCC[C@@H]6C5)n4)ccc3cn2)ccc1Cl] |
| 99 | 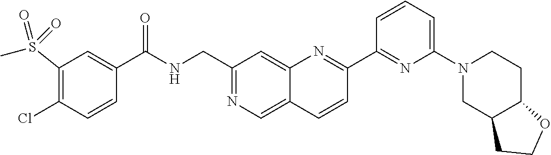 [SMILES: CS(=O)(=O)c1cc(C(=O)NCc2cc3nc(-c4cccc(N5CC[C@@H]6OCC[C@H]6C5)n4)ccc3cn2)ccc1Cl] |
| 100 | 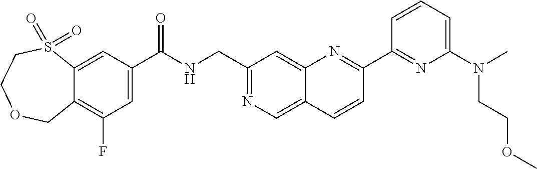 [SMILES: COCCN(C)c1cccc(-c2ccc3cnc(CNC(=O)c4cc(F)c5c(c4)S(=O)(=O)CCOC5)cc3n2)n1] |
| 101 | 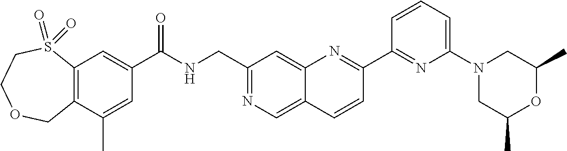 [SMILES: Cc1cc(C(=O)NCc2cc3nc(-c4cccc(N5C[C@@H](C)O[C@@H](C)C5)n4)ccc3cn2)cc2c1COCCS2(=O)=O] |
| 102 | 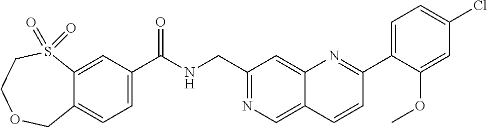 [SMILES: COc1cc(Cl)ccc1-c1ccc2cnc(CNC(=O)c3ccc4c(c3)S(=O)(=O)CCOC4)cc2n1] |
| 103 | 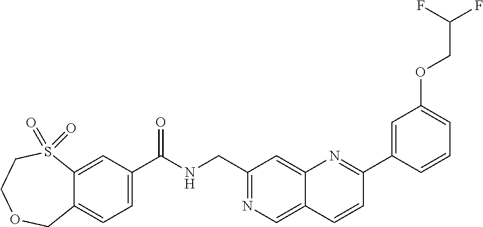 [SMILES: O=C(NCc1cc2nc(-c3cccc(OCC(F)F)c3)ccc2cn1)c1ccc2c(c1)S(=O)(=O)CCOC2] |
| 104 | 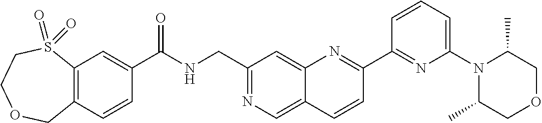 [SMILES: C[C@@H]1COC[C@H](C)N1c1cccc(-c2ccc3cnc(CNC(=O)c4ccc5c(c4)S(=O)(=O)CCOC5)cc3n2)n1] |
| 105 | 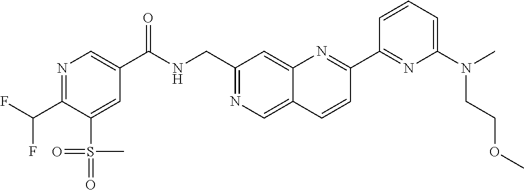 [SMILES: COCCN(C)c1cccc(-c2ccc3cnc(CNC(=O)c4cnc(C(F)F)c(S(C)(=O)=O)c4)cc3n2)n1] |
| 106 | 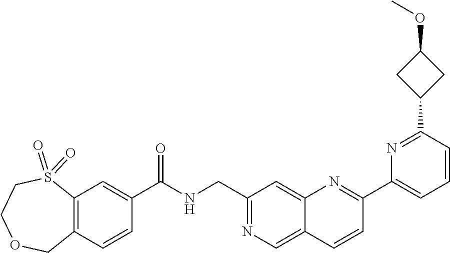 [SMILES: CO[C@H]1C[C@H](c2cccc(-c3ccc4cnc(CNC(=O)c5ccc6c(c5)S(=O)(=O)CCOC6)cc4n3)n2)C1] |
| 107 | 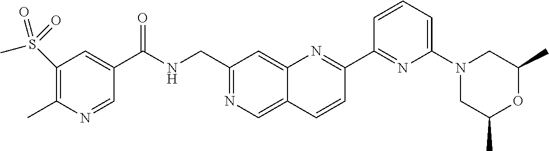 |
| 108 | 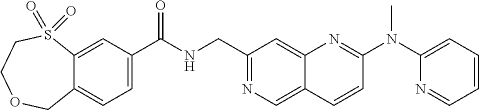 [SMILES: CN(c1ccccn1)c1ccc2cnc(CNC(=O)c3ccc4c(c3)S(=O)(=O)CCOC4)cc2n1] |
| 109 | 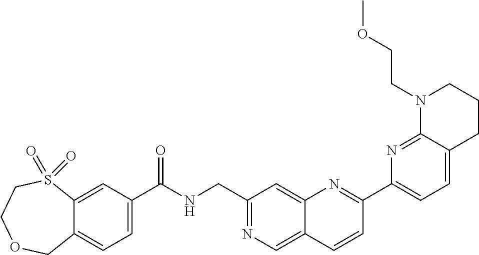 [SMILES: COCCN1CCCc2ccc(-c3ccc4cnc(CNC(=O)c5ccc6c(c5)S(=O)(=O)CCOC6)cc4n3)nc21] |
| 110 | 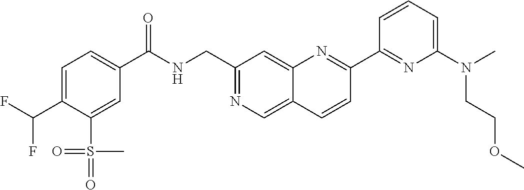 [SMILES: COCCN(C)c1cccc(-c2ccc3cnc(CNC(=O)c4ccc(C(F)F)c(S(C)(=O)=O)c4)cc3n2)n1] |
| 111 | 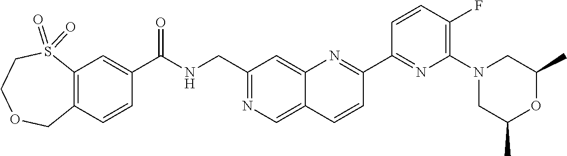 [SMILES: C[C@@H]1CN(c2nc(-c3ccc4cnc(CNC(=O)c5ccc6c(c5)S(=O)(=O)CCOC6)cc4n3)ccc2F)C[C@H](C)O1] |
| 112 | 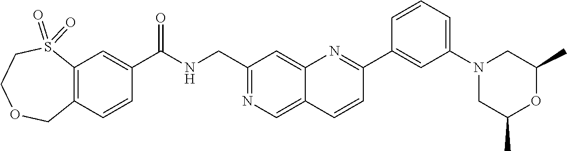 [SMILES: C[C@@H]1CN(c2cccc(-c3ccc4cnc(CNC(=O)c5ccc6c(c5)S(=O)(=O)CCOC6)cc4n3)c2)C[C@H](C)O1] |
| 113 | 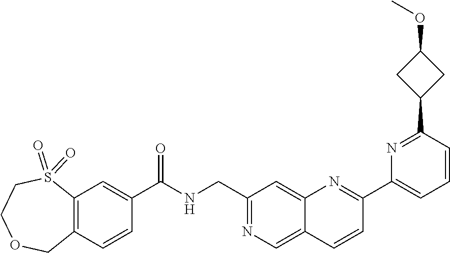 [SMILES: CO[C@H]1C[C@@H](c2cccc(-c3ccc4cnc(CNC(=O)c5ccc6c(c5)S(=O)(=O)CCOC6)cc4n3)n2)C1] |
| 114 | 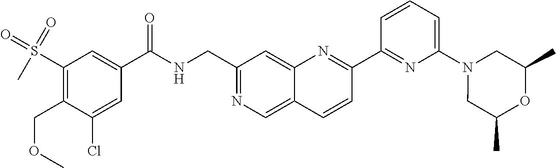 [SMILES: COCc1c(Cl)cc(C(=O)NCc2cc3nc(-c4cccc(N5C[C@@H](C)O[C@@H](C)C5)n4)ccc3cn2)cc1S(C)(=O)=O] |
| 115 | 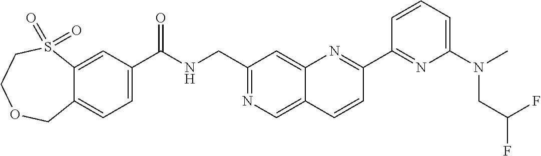 [SMILES: CN(CC(F)F)c1cccc(-c2ccc3cnc(CNC(=O)c4ccc5c(c4)S(=O)(=O)CCOC5)cc3n2)n1] |
| 116 | 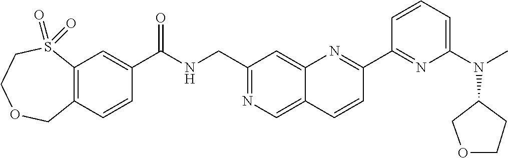 [SMILES: CN(c1cccc(-c2ccc3cnc(CNC(=O)c4ccc5c(c4)S(=O)(=O)CCOC5)cc3n2)n1)[C@@H]1CCOC1] |
| 117 | 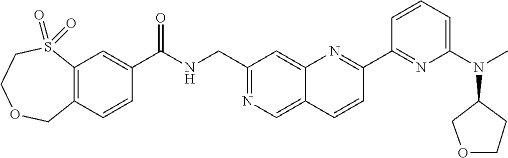 [SMILES: CN(c1cccc(-c2ccc3cnc(CNC(=O)c4ccc5c(c4)S(=O)(=O)CCOC5)cc3n2)n1)[C@H]1CCOC1] |
| 118 | 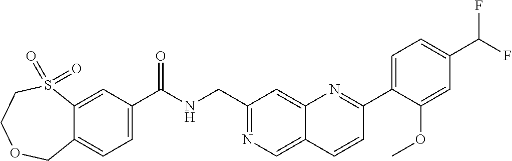 [SMILES: COc1cc(C(F)F)ccc1-c1ccc2cnc(CNC(=O)c3ccc4c(c3)S(=O)(=O)CCOC4)cc2n1] |
| 119 | 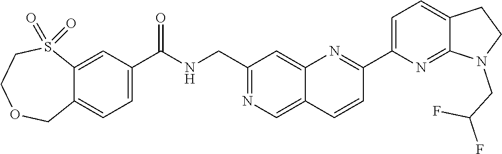 [SMILES: O=C(NCc1cc2nc(-c3ccc4c(n3)N(CC(F)F)CC4)ccc2cn1)c1ccc2c(c1)S(=O)(=O)CCOC2] |
| 120 | 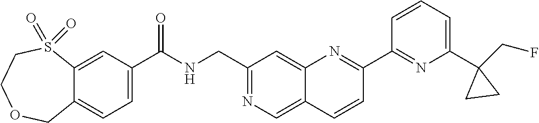 [SMILES: O=C(NCc1cc2nc(-c3cccc(C4(CF)CC4)n3)ccc2cn1)c1ccc2c(c1)S(=O)(=O)CCOC2] |
| 121 | 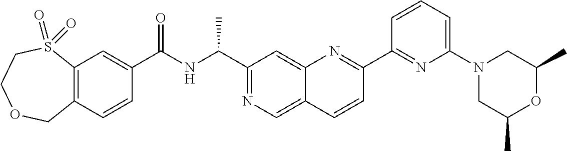 [SMILES: C[C@@H]1CN(c2cccc(-c3ccc4cnc([C@@H](C)NC(=O)c5ccc6c(c5)S(=O)(=O)CCOC6)cc4n3)n2)C[C@H](C)O1] |
| 122 | 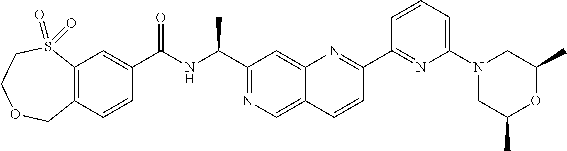 [SMILES: C[C@@H]1CN(c2cccc(-c3ccc4cnc([C@H](C)NC(=O)c5ccc6c(c5)S(=O)(=O)CCOC6)cc4n3)n2)C[C@H](C)O1] |
| 123 | 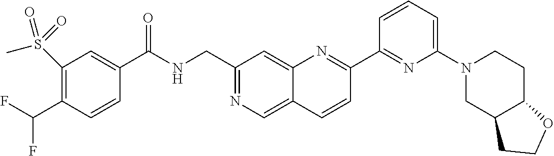 [SMILES: CS(=O)(=O)c1cc(C(=O)NCc2cc3nc(-c4cccc(N5CC[C@@H]6OCC[C@H]6C5)n4)ccc3cn2)ccc1C(F)F] |
| 124 | 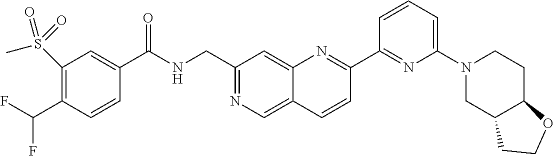 [SMILES: CS(=O)(=O)c1cc(C(=O)NCc2cc3nc(-c4cccc(N5CC[C@H]6OCC[C@@H]6C5)n4)ccc3cn2)ccc1C(F)F] |
| 125 | 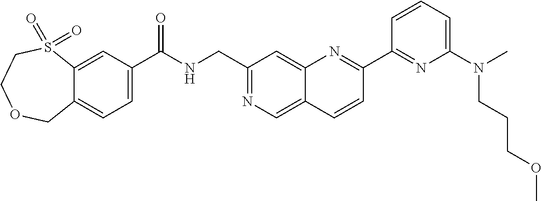 [SMILES: COCCCN(C)c1cccc(-c2ccc3cnc(CNC(=O)c4ccc5c(c4)S(=O)(=O)CCOC5)cc3n2)n1] |
| 126 | 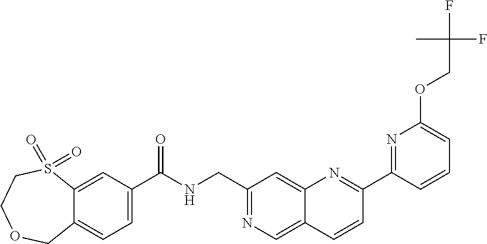 [SMILES: CC(F)(F)COc1cccc(-c2ccc3cnc(CNC(=O)c4ccc5c(c4)S(=O)(=O)CCOC5)cc3n2)n1] |
| 127 | 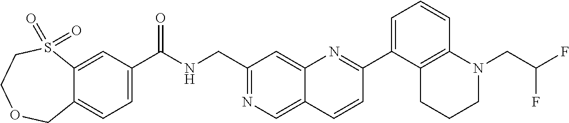 [SMILES: O=C(NCc1cc2nc(-c3cccc4c3CCCN4CC(F)F)ccc2cn1)c1ccc2c(c1)S(=O)(=O)CCOC2] |
| 128 | 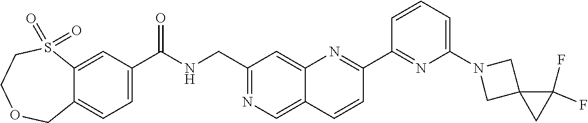 [SMILES: O=C(NCc1cc2nc(-c3cccc(N4CC5(C4)CC5(F)F)n3)ccc2cn1)c1ccc2c(c1)S(=O)(=O)CCOC2] |
| 129 | 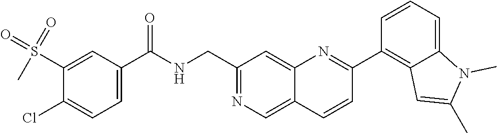 [SMILES: Cc1cc2c(-c3ccc4cnc(CNC(=O)c5ccc(Cl)c(S(C)(=O)=O)c5)cc4n3)cccc2n1C] |
| 130 | 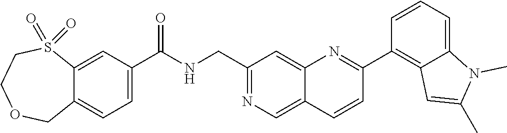 [SMILES: Cc1cc2c(-c3ccc4cnc(CNC(=O)c5ccc6c(c5)S(=O)(=O)CCOC6)cc4n3)cccc2n1C] |
| 131 | 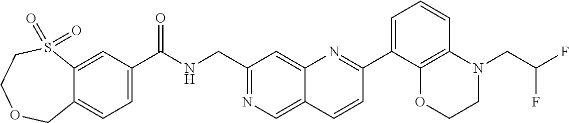 [SMILES: O=C(NCc1cc2nc(-c3cccc4c3OCCN4CC(F)F)ccc2cn1)c1ccc2c(c1)S(=O)(=O)CCOC2] |
| 132 | 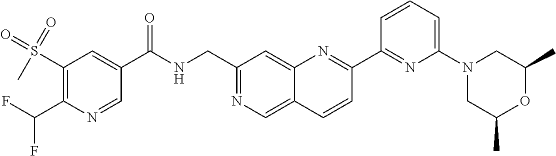 [SMILES: C[C@@H]1CN(c2cccc(-c3ccc4cnc(CNC(=O)c5cnc(C(F)F)c(S(C)(=O)=O)c5)cc4n3)n2)C[C@H](C)O1] |
| 133 | 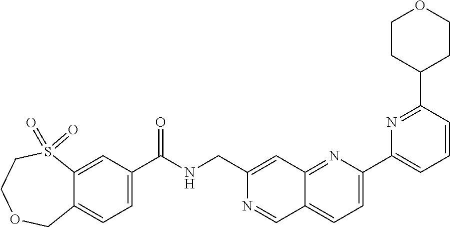 [SMILES: O=C(NCc1cc2nc(-c3cccc(C4CCOCC4)n3)ccc2cn1)c1ccc2c(c1)S(=O)(=O)CCOC2] |
| 134 | 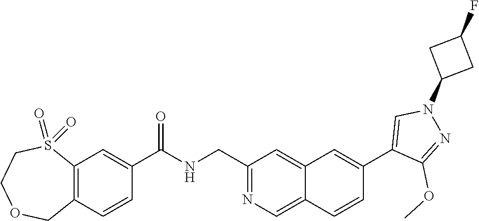 [SMILES: COc1nn([C@H]2C[C@@H](F)C2)cc1-c1ccc2cnc(CNC(=O)c3ccc4c(c3)S(=O)(=O)CCOC4)cc2c1] |
| 135 | 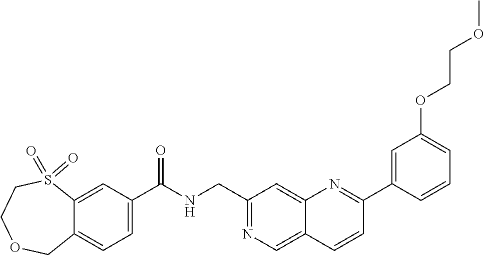 [SMILES: COCCOc1cccc(-c2ccc3cnc(CNC(=O)c4ccc5c(c4)S(=O)(=O)CCOC5)cc3n2)c1] |
| 136 | 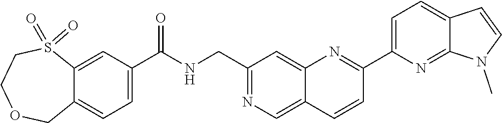 [SMILES: Cn1ccc2ccc(-c3ccc4cnc(CNC(=O)c5ccc6c(c5)S(=O)(=O)CCOC6)cc4n3)nc21] |
| 137 | 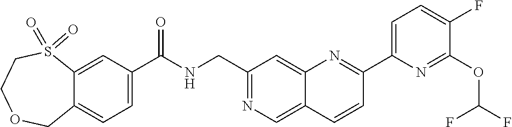 [SMILES: O=C(NCc1cc2nc(-c3ccc(F)c(OC(F)F)n3)ccc2cn1)c1ccc2c(c1)S(=O)(=O)CCOC2] |
| 138 | 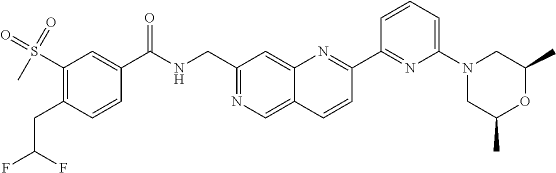 [SMILES: C[C@@H]1CN(c2cccc(-c3ccc4cnc(CNC(=O)c5ccc(CC(F)F)c(S(C)(=O)=O)c5)cc4n3)n2)C[C@H](C)O1] |
| 139 | 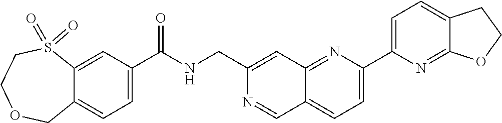 [SMILES: O=C(NCc1cc2nc(-c3ccc4c(n3)OCC4)ccc2cn1)c1ccc2c(c1)S(=O)(=O)CCOC2] |
| 140 | 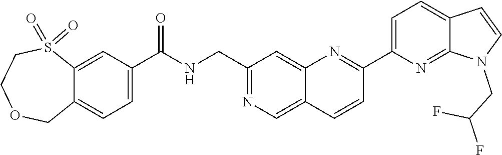 [SMILES: O=C(NCc1cc2nc(-c3ccc4ccn(CC(F)F)c4n3)ccc2cn1)c1ccc2c(c1)S(=O)(=O)CCOC2] |
| 141 | 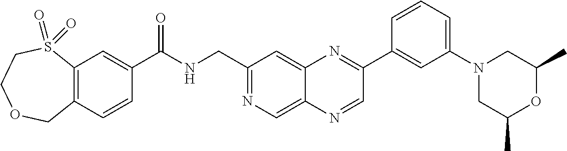 [SMILES: C[C@@H]1CN(c2cccc(-c3cnc4cnc(CNC(=O)c5ccc6c(c5)S(=O)(=O)CCOC6)cc4n3)c2)C[C@H](C)O1] |
| 142 | 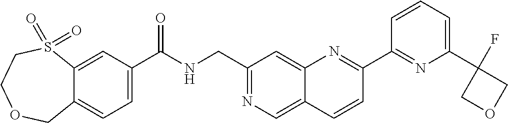 [SMILES: O=C(NCc1cc2nc(-c3cccc(C4(F)COC4)n3)ccc2cn1)c1ccc2c(c1)S(=O)(=O)CCOC2] |
| 143 | 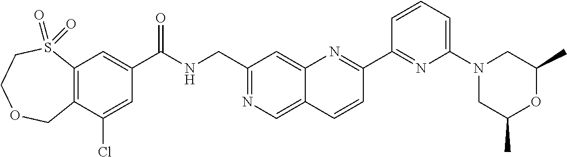 [SMILES: C[C@@H]1CN(c2cccc(-c3ccc4cnc(CNC(=O)c5cc(Cl)c6c(c5)S(=O)(=O)CCOC6)cc4n3)n2)C[C@H](C)O1] |
| 144 | 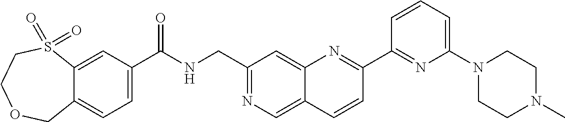 [SMILES: CN1CCN(c2cccc(-c3ccc4cnc(CNC(=O)c5ccc6c(c5)S(=O)(=O)CCOC6)cc4n3)n2)CC1] |
| 145 | 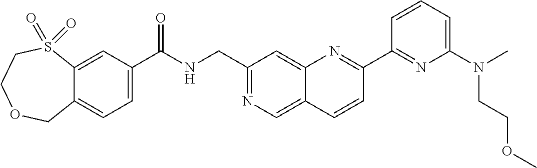 [SMILES: COCCN(C)c1cccc(-c2ccc3cnc(CNC(=O)c4ccc5c(c4)S(=O)(=O)CCOC5)cc3n2)n1] |
| 146 | 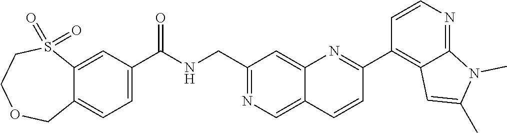 [SMILES: Cc1cc2c(-c3ccc4cnc(CNC(=O)c5ccc6c(c5)S(=O)(=O)CCOC6)cc4n3)ccnc2n1C] |
| 147 | 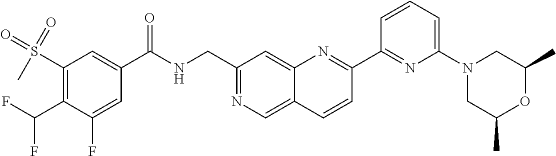 [SMILES: C[C@@H]1CN(c2cccc(-c3ccc4cnc(CNC(=O)c5cc(F)c(C(F)F)c(S(C)(=O)=O)c5)cc4n3)n2)C[C@H](C)O1] |
| 148 | 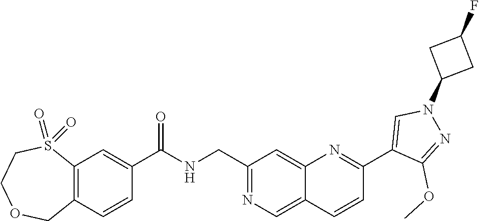 [SMILES: COc1nn([C@H]2C[C@@H](F)C2)cc1-c1ccc2cnc(CNC(=O)c3ccc4c(c3)S(=O)(=O)CCOC4)cc2n1] |
| 149 | 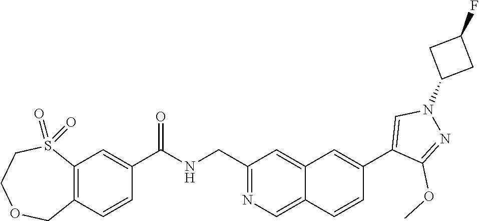 [SMILES: COc1nn([C@H]2C[C@H](F)C2)cc1-c1ccc2cnc(CNC(=O)c3ccc4c(c3)S(=O)(=O)CCOC4)cc2c1] |
| 150 | 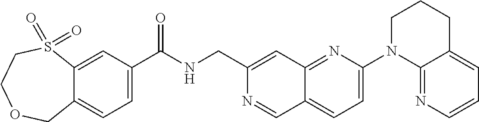 [SMILES: O=C(NCc1cc2nc(N3CCCc4cccnc43)ccc2cn1)c1ccc2c(c1)S(=O)(=O)CCOC2] |
| 151 | 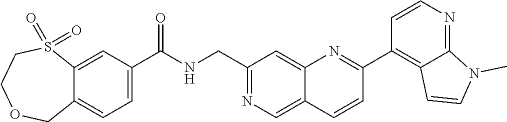 [SMILES: Cn1ccc2c(-c3ccc4cnc(CNC(=O)c5ccc6c(c5)S(=O)(=O)CCOC6)cc4n3)ccnc21] |
| 152 | 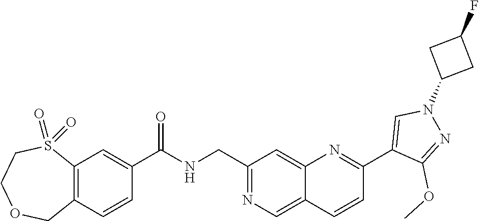 [SMILES: COc1nn([C@H]2C[C@H](F)C2)cc1-c1ccc2cnc(CNC(=O)c3ccc4c(c3)S(=O)(=O)CCOC4)cc2n1] |
| 153 | 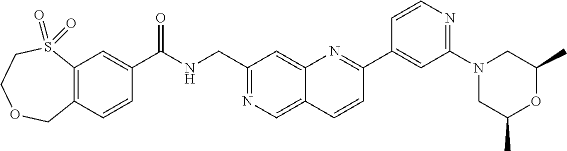 [SMILES: C[C@@H]1CN(c2cc(-c3ccc4cnc(CNC(=O)c5ccc6c(c5)S(=O)(=O)CCOC6)cc4n3)ccn2)C[C@H](C)O1] |
| 154 | 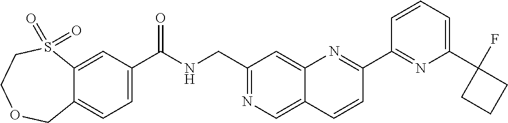 [SMILES: O=C(NCc1cc2nc(-c3cccc(C4(F)CCC4)n3)ccc2cn1)c1ccc2c(c1)S(=O)(=O)CCOC2] |
| 155 | 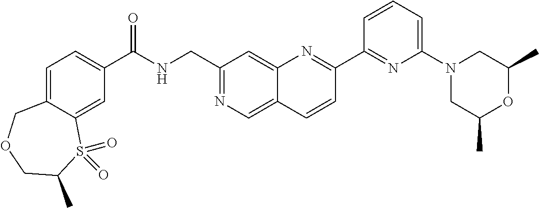 [SMILES: C[C@@H]1CN(c2cccc(-c3ccc4cnc(CNC(=O)c5ccc6c(c5)S(=O)(=O)[C@@H](C)COC6)cc4n3)n2)C[C@H](C)O1] |
| 156 | 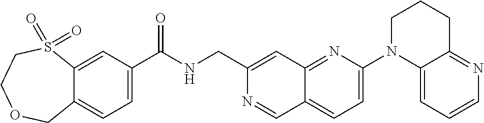 [SMILES: O=C(NCc1cc2nc(N3CCCc4ncccc43)ccc2cn1)c1ccc2c(c1)S(=O)(=O)CCOC2] |
| 157 | 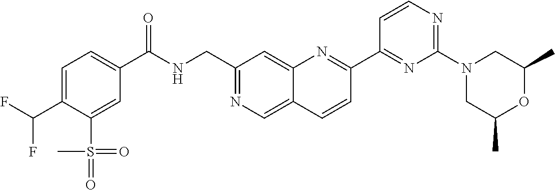 [SMILES: C[C@@H]1CN(c2nccc(-c3ccc4cnc(CNC(=O)c5ccc(C(F)F)c(S(C)(=O)=O)c5)cc4n3)n2)C[C@H](C)O1] |
| 158 | 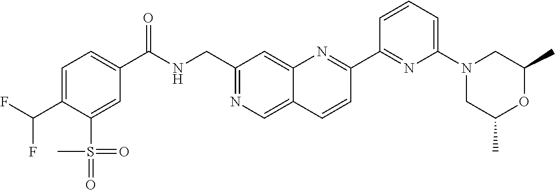 [SMILES: C[C@@H]1CN(c2cccc(-c3ccc4cnc(CNC(=O)c5ccc(C(F)F)c(S(C)(=O)=O)c5)cc4n3)n2)C[C@@H](C)O1] |
| 159 | 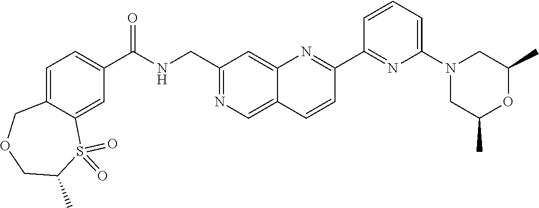 [SMILES: C[C@@H]1CN(c2cccc(-c3ccc4cnc(CNC(=O)c5ccc6c(c5)S(=O)(=O)[C@H](C)COC6)cc4n3)n2)C[C@H](C)O1] |
| 160 | 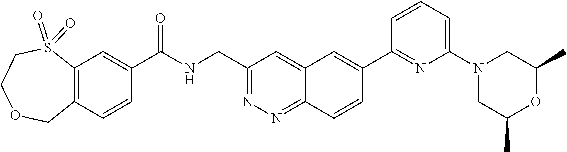 [SMILES: C[C@@H]1CN(c2cccc(-c3ccc4nnc(CNC(=O)c5ccc6c(c5)S(=O)(=O)CCOC6)cc4c3)n2)C[C@H](C)O1] |
| 161 | 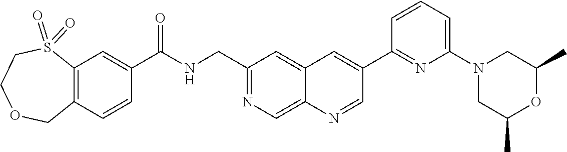 [SMILES: C[C@@H]1CN(c2cccc(-c3cnc4cnc(CNC(=O)c5ccc6c(c5)S(=O)(=O)CCOC6)cc4c3)n2)C[C@H](C)O1] |
| 162 | 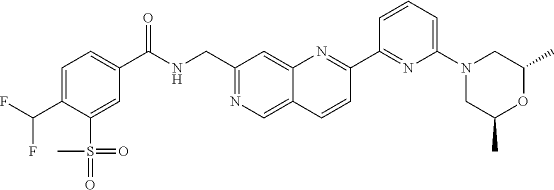 [SMILES: C[C@H]1CN(c2cccc(-c3ccc4cnc(CNC(=O)c5ccc(C(F)F)c(S(C)(=O)=O)c5)cc4n3)n2)C[C@H](C)O1] |
| 163 | 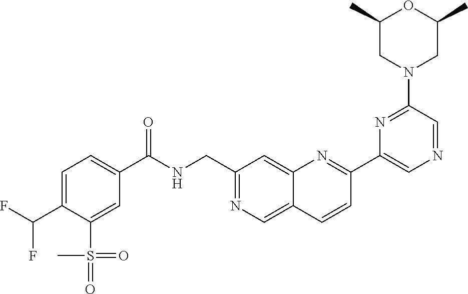 [SMILES: C[C@@H]1CN(c2cncc(-c3ccc4cnc(CNC(=O)c5ccc(C(F)F)c(S(C)(=O)=O)c5)cc4n3)n2)C[C@H](C)O1] |
| 164 | 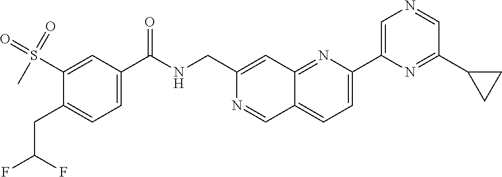 [SMILES: CS(=O)(=O)c1cc(C(=O)NCc2cc3nc(-c4cncc(C5CC5)n4)ccc3cn2)ccc1CC(F)F] |
| 165 | 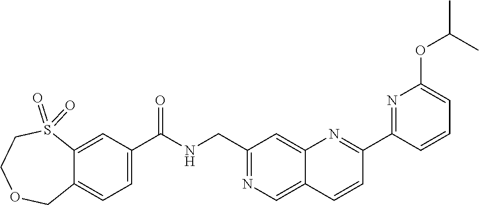 [SMILES: CC(C)Oc1cccc(-c2ccc3cnc(CNC(=O)c4ccc5c(c4)S(=O)(=O)CCOC5)cc3n2)n1] |
| 166 | 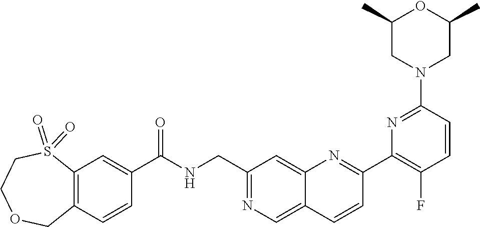 [SMILES: C[C@@H]1CN(c2ccc(F)c(-c3ccc4cnc(CNC(=O)c5ccc6c(c5)S(=O)(=O)CCOC6)cc4n3)n2)C[C@H](C)O1] |
| 167 | 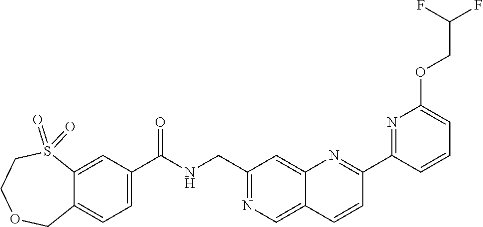 [SMILES: O=C(NCc1cc2nc(-c3cccc(OCC(F)F)n3)ccc2cn1)c1ccc2c(c1)S(=O)(=O)CCOC2] |
| 168 | 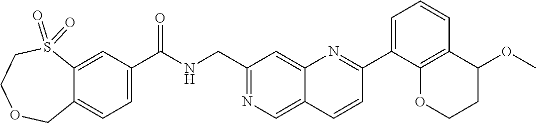 [SMILES: COC1CCOc2c(-c3ccc4cnc(CNC(=O)c5ccc6c(c5)S(=O)(=O)CCOC6)cc4n3)cccc21] |
| 169 | 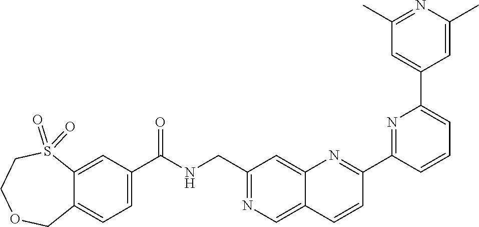 [SMILES: Cc1cc(-c2cccc(-c3ccc4cnc(CNC(=O)c5ccc6c(c5)S(=O)(=O)CCOC6)cc4n3)n2)cc(C)n1] |
| 170 | 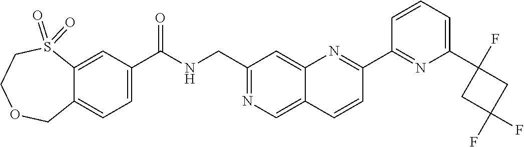 [SMILES: O=C(NCc1cc2nc(-c3cccc(C4(F)CC(F)(F)C4)n3)ccc2cn1)c1ccc2c(c1)S(=O)(=O)CCOC2] |
| 171 | 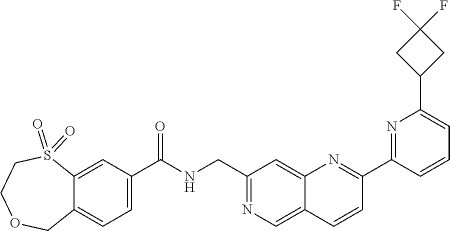 [SMILES: O=C(NCc1cc2nc(-c3cccc(C4CC(F)(F)C4)n3)ccc2cn1)c1ccc2c(c1)S(=O)(=O)CCOC2] |
| 172 | 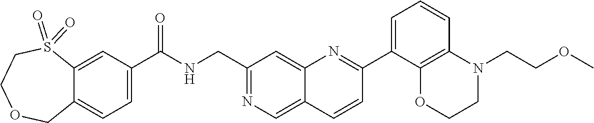 [SMILES: COCCN1CCOc2c(-c3ccc4cnc(CNC(=O)c5ccc6c(c5)S(=O)(=O)CCOC6)cc4n3)cccc21] |
| 173 | 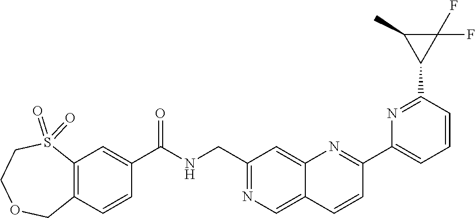 [SMILES: C[C@@H]1[C@@H](c2cccc(-c3ccc4cnc(CNC(=O)c5ccc6c(c5)S(=O)(=O)CCOC6)cc4n3)n2)C1(F)F] |
| 174 | 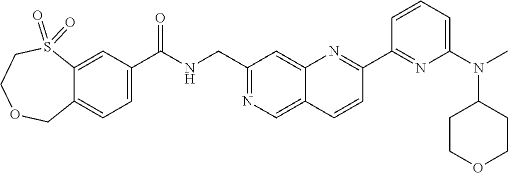 [SMILES: CN(c1cccc(-c2ccc3cnc(CNC(=O)c4ccc5c(c4)S(=O)(=O)CCOC5)cc3n2)n1)C1CCOCC1] |
| 175 | 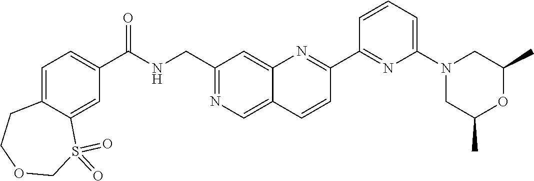 [SMILES: C[C@@H]1CN(c2cccc(-c3ccc4cnc(CNC(=O)c5ccc6c(c5)S(=O)(=O)COCC6)cc4n3)n2)C[C@H](C)O1] |
| 176 | 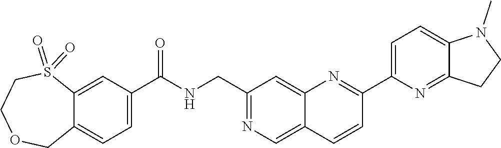 [SMILES: CN1CCc2nc(-c3ccc4cnc(CNC(=O)c5ccc6c(c5)S(=O)(=O)CCOC6)cc4n3)ccc21] |
| 177 | 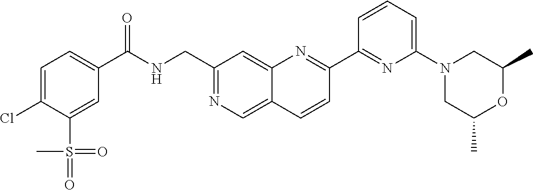 [SMILES: C[C@@H]1CN(c2cccc(-c3ccc4cnc(CNC(=O)c5ccc(Cl)c(S(C)(=O)=O)c5)cc4n3)n2)C[C@@H](C)O1] |
| 178 | 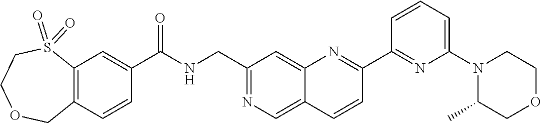 [SMILES: C[C@H]1COCCN1c1cccc(-c2ccc3cnc(CNC(=O)c4ccc5c(c4)S(=O)(=O)CCOC5)cc3n2)n1] |
| 179 | 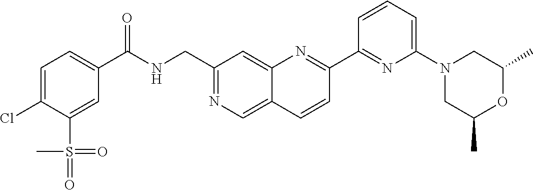 [SMILES: C[C@H]1CN(c2cccc(-c3ccc4cnc(CNC(=O)c5ccc(Cl)c(S(C)(=O)=O)c5)cc4n3)n2)C[C@H](C)O1] |
| 180 | 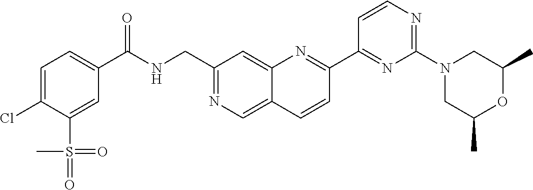 [SMILES: C[C@@H]1CN(c2nccc(-c3ccc4cnc(CNC(=O)c5ccc(Cl)c(S(C)(=O)=O)c5)cc4n3)n2)C[C@H](C)O1] |
| 181 | 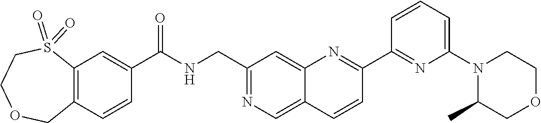 [SMILES: C[C@@H]1COCCN1c1cccc(-c2ccc3cnc(CNC(=O)c4ccc5c(c4)S(=O)(=O)CCOC5)cc3n2)n1] |
| 182 | 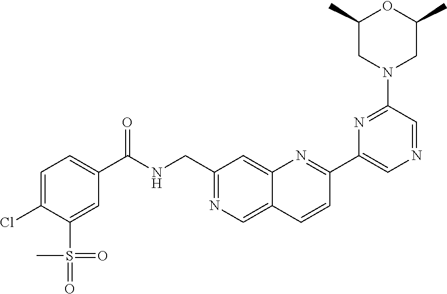 [SMILES: C[C@@H]1CN(c2cncc(-c3ccc4cnc(CNC(=O)c5ccc(Cl)c(S(C)(=O)=O)c5)cc4n3)n2)C[C@H](C)O1] |
| 183 | 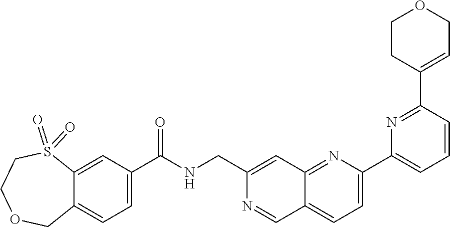 [SMILES: O=C(NCc1cc2nc(-c3cccc(C4=CCOCC4)n3)ccc2cn1)c1ccc2c(c1)S(=O)(=O)CCOC2] |
| 184 | 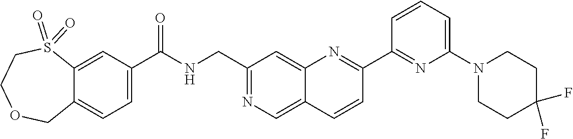 [SMILES: O=C(NCc1cc2nc(-c3cccc(N4CCC(F)(F)CC4)n3)ccc2cn1)c1ccc2c(c1)S(=O)(=O)CCOC2] |
| 185 | 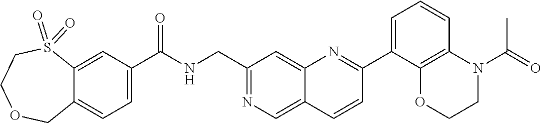 [SMILES: CC(=O)N1CCOc2c(-c3ccc4cnc(CNC(=O)c5ccc6c(c5)S(=O)(=O)CCOC6)cc4n3)cccc21] |
| 186 | 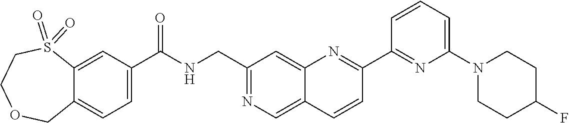 [SMILES: O=C(NCc1cc2nc(-c3cccc(N4CCC(F)CC4)n3)ccc2cn1)c1ccc2c(c1)S(=O)(=O)CCOC2] |
| 187 | 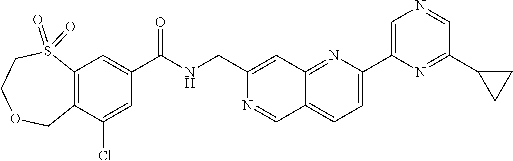 [SMILES: O=C(NCc1cc2nc(-c3cncc(C4CC4)n3)ccc2cn1)c1cc(Cl)c2c(c1)S(=O)(=O)CCOC2] |
| 188 | 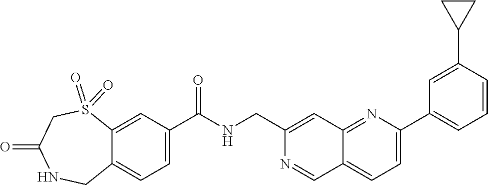 [SMILES: O=C1CS(=O)(=O)c2cc(C(=O)NCc3cc4nc(-c5cccc(C6CC6)c5)ccc4cn3)ccc2CN1] |
| 189 | 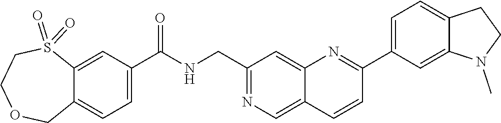 [SMILES: CN1CCc2ccc(-c3ccc4cnc(CNC(=O)c5ccc6c(c5)S(=O)(=O)CCOC6)cc4n3)cc21] |
| 190 | 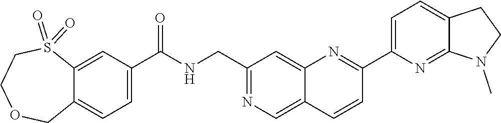 [SMILES: CN1CCc2ccc(-c3ccc4cnc(CNC(=O)c5ccc6c(c5)S(=O)(=O)CCOC6)cc4n3)nc21] |
| 191 | 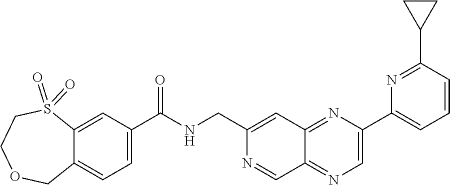 [SMILES: O=C(NCc1cc2nc(-c3cccc(C4CC4)n3)cnc2cn1)c1ccc2c(c1)S(=O)(=O)CCOC2] |
| 192 | 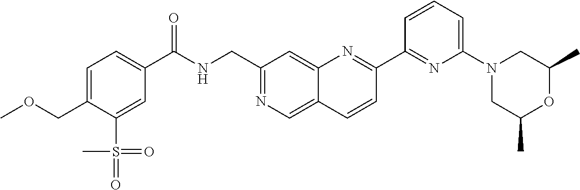 |
| 193 | 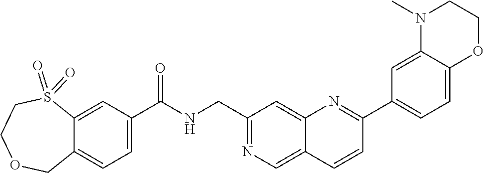 [SMILES: CN1CCOc2ccc(-c3ccc4cnc(CNC(=O)c5ccc6c(c5)S(=O)(=O)CCOC6)cc4n3)cc21] |
| 194 | 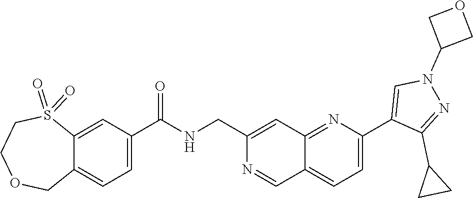 [SMILES: O=C(NCc1cc2nc(-c3cn(C4COC4)nc3C3CC3)ccc2cn1)c1ccc2c(c1)S(=O)(=O)CCOC2] |
| 195 | 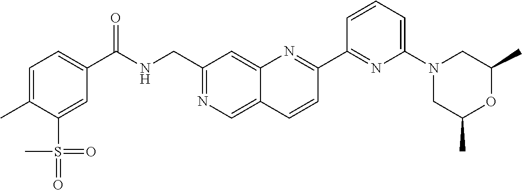 |
| 196 | 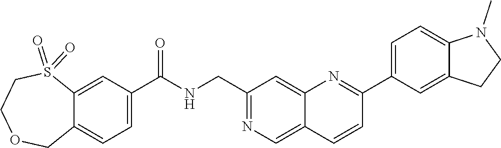 [SMILES: CN1CCc2cc(-c3ccc4cnc(CNC(=O)c5ccc6c(c5)S(=O)(=O)CCOC6)cc4n3)ccc21] |
| 197 | 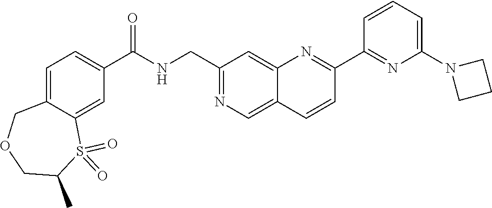 [SMILES: C[C@H]1COCc2ccc(C(=O)NCc3cc4nc(-c5cccc(N6CCC6)n5)ccc4cn3)cc2S1(=O)=O] |
| 198 | 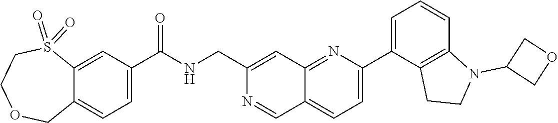 [SMILES: O=C(NCc1cc2nc(-c3cccc4c3CCN4C3COC3)ccc2cn1)c1ccc2c(c1)S(=O)(=O)CCOC2] |
| 199 | 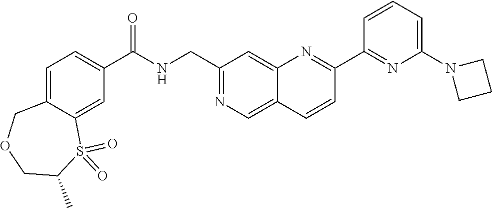 [SMILES: C[C@@H]1COCc2ccc(C(=O)NCc3cc4nc(-c5cccc(N6CCC6)n5)ccc4cn3)cc2S1(=O)=O] |
| 200 | 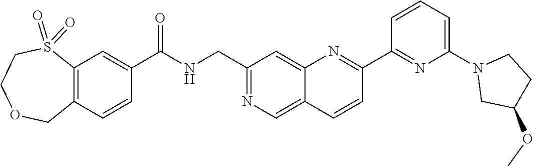 [SMILES: CO[C@@H]1CCN(c2cccc(-c3ccc4cnc(CNC(=O)c5ccc6c(c5)S(=O)(=O)CCOC6)cc4n3)n2)C1] |
| 201 | 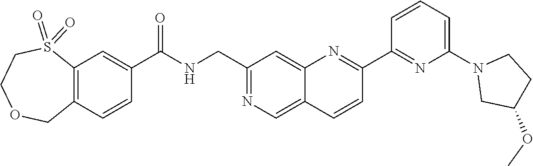 [SMILES: CO[C@H]1CCN(c2cccc(-c3ccc4cnc(CNC(=O)c5ccc6c(c5)S(=O)(=O)CCOC6)cc4n3)n2)C1] |
| 202 | 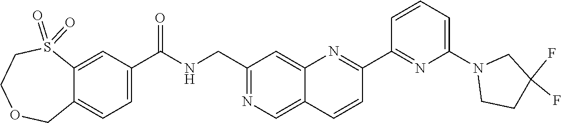 [SMILES: O=C(NCc1cc2nc(-c3cccc(N4CCC(F)(F)C4)n3)ccc2cn1)c1ccc2c(c1)S(=O)(=O)CCOC2] |
| 203 | 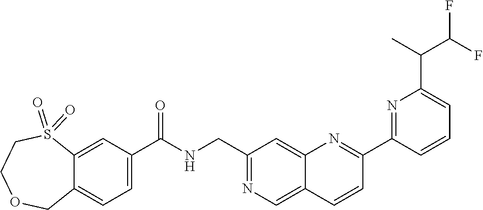 [SMILES: CC(c1cccc(-c2ccc3cnc(CNC(=O)c4ccc5c(c4)S(=O)(=O)CCOC5)cc3n2)n1)C(F)F] |
| 204 | 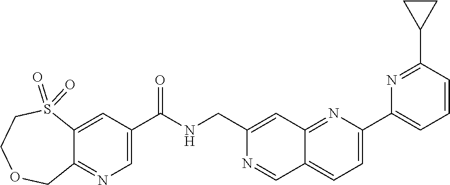 [SMILES: O=C(NCc1cc2nc(-c3cccc(C4CC4)n3)ccc2cn1)c1cnc2c(c1)S(=O)(=O)CCOC2] |
| 205 | 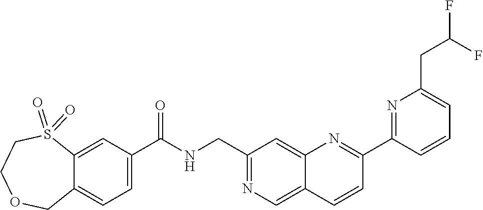 [SMILES: O=C(NCc1cc2nc(-c3cccc(CC(F)F)n3)ccc2cn1)c1ccc2c(c1)S(=O)(=O)CCOC2] |
| 206 | 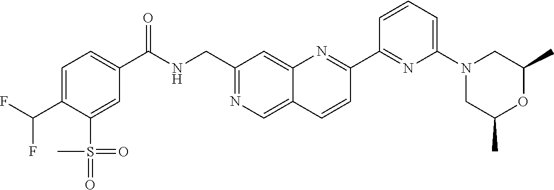 [SMILES: C[C@@H]1CN(c2cccc(-c3ccc4cnc(CNC(=O)c5ccc(C(F)F)c(S(C)(=O)=O)c5)cc4n3)n2)C[C@H](C)O1] |
| 207 | 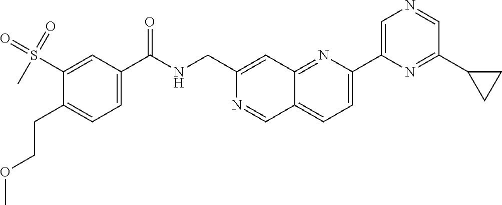 [SMILES: COCCc1ccc(C(=O)NCc2cc3nc(-c4cncc(C5CC5)n4)ccc3cn2)cc1S(C)(=O)=O] |
| 208 | 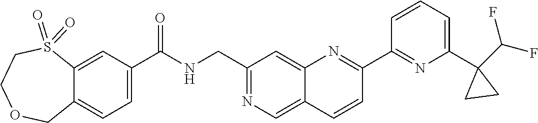 [SMILES: O=C(NCc1cc2nc(-c3cccc(C4(C(F)F)CC4)n3)ccc2cn1)c1ccc2c(c1)S(=O)(=O)CCOC2] |
| 209 | 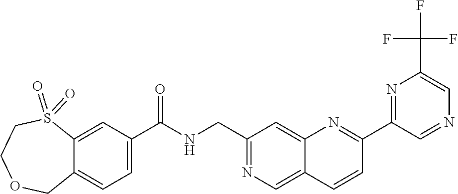 [SMILES: O=C(NCc1cc2nc(-c3cncc(C(F)(F)F)n3)ccc2cn1)c1ccc2c(c1)S(=O)(=O)CCOC2] |
| 210 | 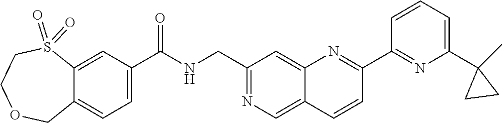 [SMILES: CC1(c2cccc(-c3ccc4cnc(CNC(=O)c5ccc6c(c5)S(=O)(=O)CCOC6)cc4n3)n2)CC1] |
| 211 | 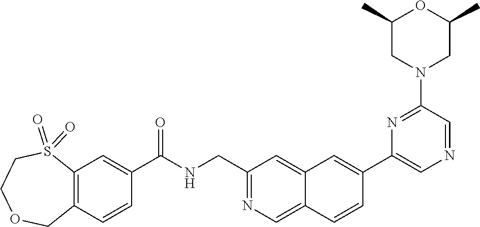 [SMILES: C[C@@H]1CN(c2cncc(-c3ccc4cnc(CNC(=O)c5ccc6c(c5)S(=O)(=O)CCOC6)cc4c3)n2)C[C@H](C)O1] |
| 212 | 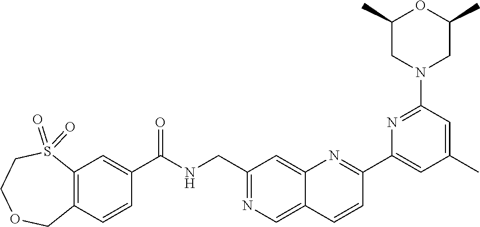 [SMILES: Cc1cc(-c2ccc3cnc(CNC(=O)c4ccc5c(c4)S(=O)(=O)CCOC5)cc3n2)nc(N2C[C@@H](C)O[C@@H](C)C2)c1] |
| 213 | 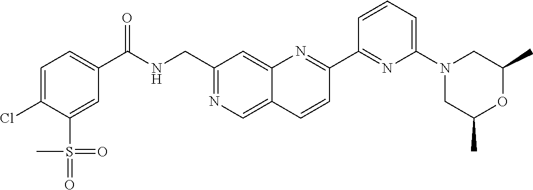 [SMILES: C[C@@H]1CN(c2cccc(-c3ccc4cnc(CNC(=O)c5ccc(Cl)c(S(C)(=O)=O)c5)cc4n3)n2)C[C@H](C)O1] |
| 214 | 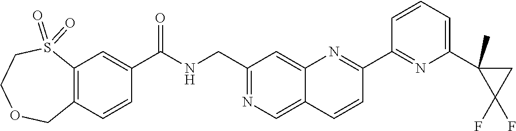 [SMILES: C[C@@]1(c2cccc(-c3ccc4cnc(CNC(=O)c5ccc6c(c5)S(=O)(=O)CCOC6)cc4n3)n2)CC1(F)F] |
| 215 | 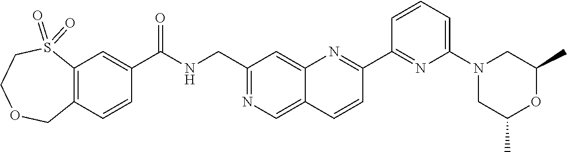 [SMILES: C[C@@H]1CN(c2cccc(-c3ccc4cnc(CNC(=O)c5ccc6c(c5)S(=O)(=O)CCOC6)cc4n3)n2)C[C@@H](C)O1] |
| 216 | 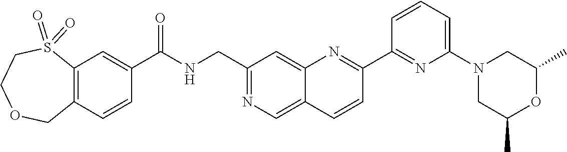 [SMILES: C[C@H]1CN(c2cccc(-c3ccc4cnc(CNC(=O)c5ccc6c(c5)S(=O)(=O)CCOC6)cc4n3)n2)C[C@H](C)O1] |
| 217 | 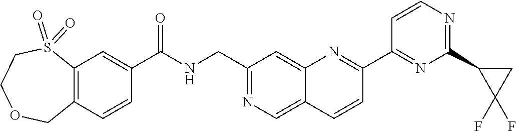 [SMILES: O=C(NCc1cc2nc(-c3ccnc([C@H]4CC4(F)F)n3)ccc2cn1)c1ccc2c(c1)S(=O)(=O)CCOC2] |
| 218 | 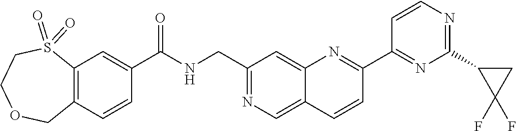 [SMILES: O=C(NCc1cc2nc(-c3ccnc([C@@H]4CC4(F)F)n3)ccc2cn1)c1ccc2c(c1)S(=O)(=O)CCOC2] |
| 219 | 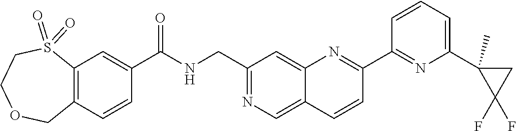 [SMILES: C[C@]1(c2cccc(-c3ccc4cnc(CNC(=O)c5ccc6c(c5)S(=O)(=O)CCOC6)cc4n3)n2)CC1(F)F] |
| 220 | 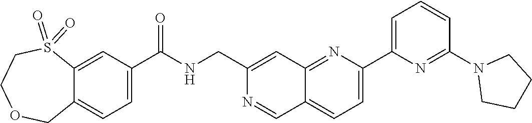 [SMILES: O=C(NCc1cc2nc(-c3cccc(N4CCCC4)n3)ccc2cn1)c1ccc2c(c1)S(=O)(=O)CCOC2] |
| 221 | 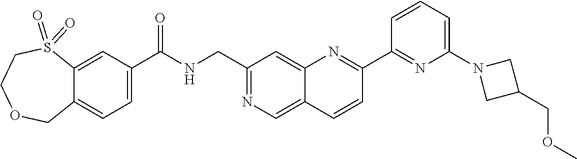 [SMILES: COCC1CN(c2cccc(-c3ccc4cnc(CNC(=O)c5ccc6c(c5)S(=O)(=O)CCOC6)cc4n3)n2)C1] |
| 222 | 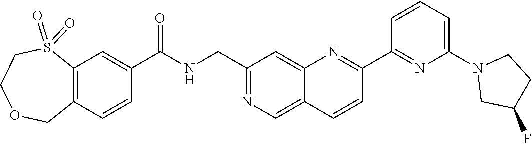 [SMILES: O=C(NCc1cc2nc(-c3cccc(N4CC[C@@H](F)C4)n3)ccc2cn1)c1ccc2c(c1)S(=O)(=O)CCOC2] |
| 223 | 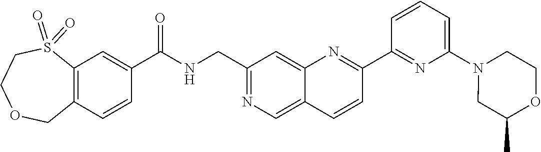 [SMILES: C[C@H]1CN(c2cccc(-c3ccc4cnc(CNC(=O)c5ccc6c(c5)S(=O)(=O)CCOC6)cc4n3)n2)CCO1] |
| 224 | 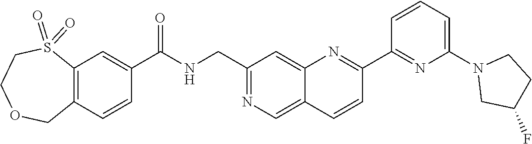 [SMILES: O=C(NCc1cc2nc(-c3cccc(N4CC[C@H](F)C4)n3)ccc2cn1)c1ccc2c(c1)S(=O)(=O)CCOC2] |
| 225 | 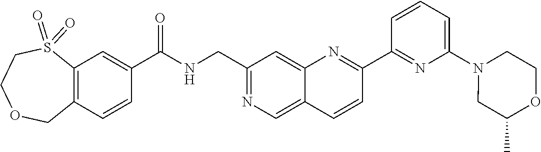 [SMILES: C[C@@H]1CN(c2cccc(-c3ccc4cnc(CNC(=O)c5ccc6c(c5)S(=O)(=O)CCOC6)cc4n3)n2)CCO1] |
| 226 | 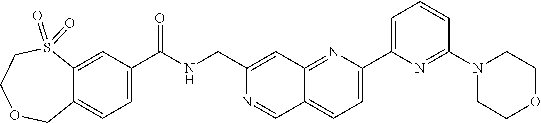 [SMILES: O=C(NCc1cc2nc(-c3cccc(N4CCOCC4)n3)ccc2cn1)c1ccc2c(c1)S(=O)(=O)CCOC2] |
| 227 | 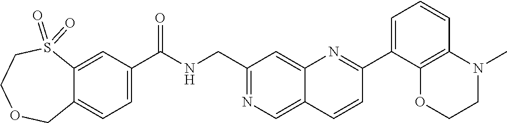 [SMILES: CN1CCOc2c(-c3ccc4cnc(CNC(=O)c5ccc6c(c5)S(=O)(=O)CCOC6)cc4n3)cccc21] |
| 228 | 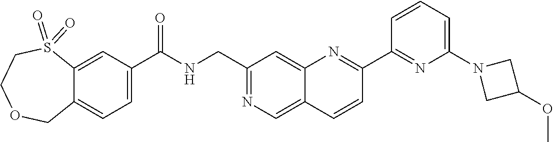 [SMILES: COC1CN(c2cccc(-c3ccc4cnc(CNC(=O)c5ccc6c(c5)S(=O)(=O)CCOC6)cc4n3)n2)C1] |
| 229 | 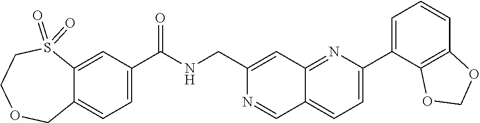 [SMILES: O=C(NCc1cc2nc(-c3cccc4c3OCO4)ccc2cn1)c1ccc2c(c1)S(=O)(=O)CCOC2] |
| 230 | 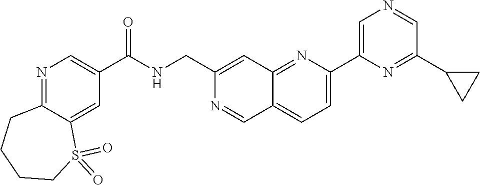 [SMILES: O=C(NCc1cc2nc(-c3cncc(C4CC4)n3)ccc2cn1)c1cnc2c(c1)S(=O)(=O)CCCC2] |
| 231 | 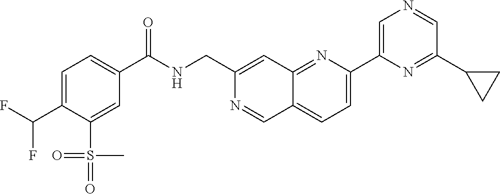 [SMILES: CS(=O)(=O)c1cc(C(=O)NCc2cc3nc(-c4cncc(C5CC5)n4)ccc3cn2)ccc1C(F)F] |
| 232 | 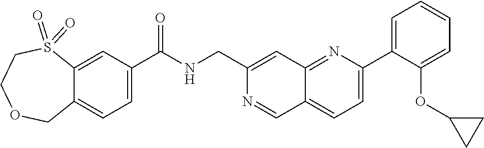 [SMILES: O=C(NCc1cc2nc(-c3ccccc3OC3CC3)ccc2cn1)c1ccc2c(c1)S(=O)(=O)CCOC2] |
| 233 | 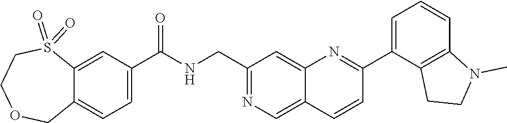 [SMILES: CN1CCc2c(-c3ccc4cnc(CNC(=O)c5ccc6c(c5)S(=O)(=O)CCOC6)cc4n3)cccc21] |
| 234 | 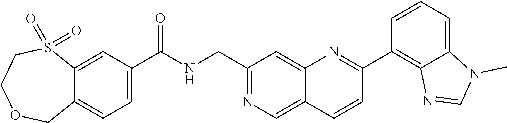 [SMILES: Cn1cnc2c(-c3ccc4cnc(CNC(=O)c5ccc6c(c5)S(=O)(=O)CCOC6)cc4n3)cccc21] |
| 235 | 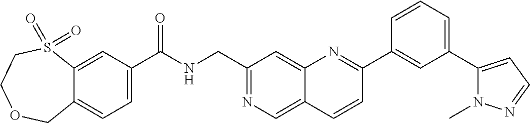 [SMILES: Cn1nccc1-c1cccc(-c2ccc3cnc(CNC(=O)c4ccc5c(c4)S(=O)(=O)CCOC5)cc3n2)c1] |
| 236 | 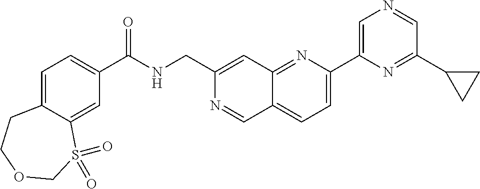 [SMILES: O=C(NCc1cc2nc(-c3cncc(C4CC4)n3)ccc2cn1)c1ccc2c(c1)S(=O)(=O)COCC2] |
| 237 | 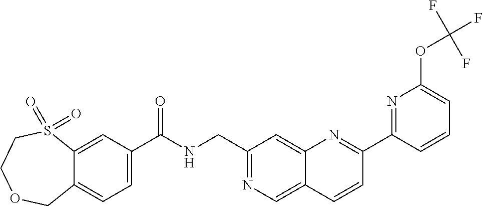 [SMILES: O=C(NCc1cc2nc(-c3cccc(OC(F)(F)F)n3)ccc2cn1)c1ccc2c(c1)S(=O)(=O)CCOC2] |
| 238 | 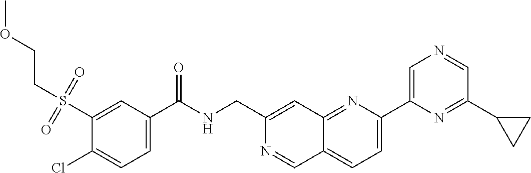 [SMILES: COCCS(=O)(=O)c1cc(C(=O)NCc2cc3nc(-c4cncc(C5CC5)n4)ccc3cn2)ccc1Cl] |
| 239 | 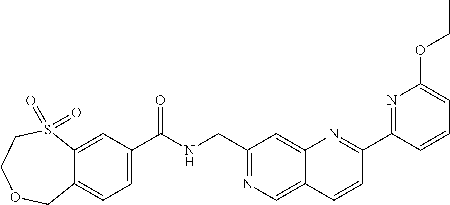 [SMILES: CCOc1cccc(-c2ccc3cnc(CNC(=O)c4ccc5c(c4)S(=O)(=O)CCOC5)cc3n2)n1] |
| 240 | 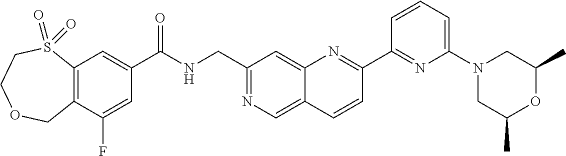 [SMILES: C[C@@H]1CN(c2cccc(-c3ccc4cnc(CNC(=O)c5cc(F)c6c(c5)S(=O)(=O)CCOC6)cc4n3)n2)C[C@H](C)O1] |
| 241 | 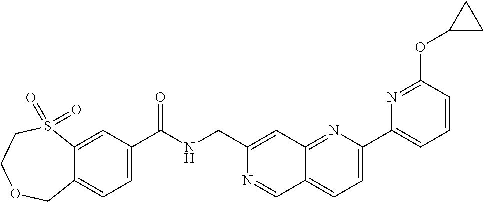 [SMILES: O=C(NCc1cc2nc(-c3cccc(OC4CC4)n3)ccc2cn1)c1ccc2c(c1)S(=O)(=O)CCOC2] |
| 242 | 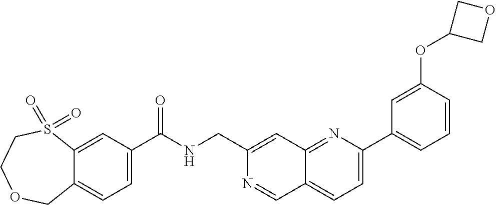 [SMILES: O=C(NCc1cc2nc(-c3cccc(OC4COC4)c3)ccc2cn1)c1ccc2c(c1)S(=O)(=O)CCOC2] |
| 243 | 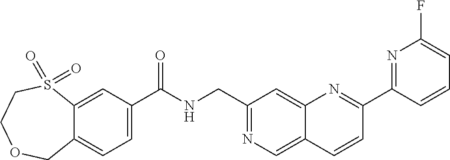 [SMILES: O=C(NCc1cc2nc(-c3cccc(F)n3)ccc2cn1)c1ccc2c(c1)S(=O)(=O)CCOC2] |
| 244 | 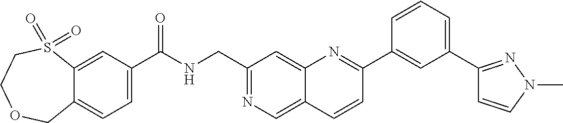 [SMILES: Cn1ccc(-c2cccc(-c3ccc4cnc(CNC(=O)c5ccc6c(c5)S(=O)(=O)CCOC6)cc4n3)c2)n1] |
| 245 | 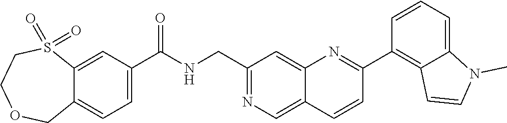 [SMILES: Cn1ccc2c(-c3ccc4cnc(CNC(=O)c5ccc6c(c5)S(=O)(=O)CCOC6)cc4n3)cccc21] |
| 246 | 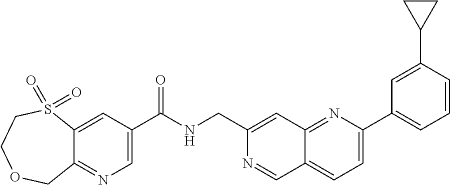 [SMILES: O=C(NCc1cc2nc(-c3cccc(C4CC4)c3)ccc2cn1)c1cnc2c(c1)S(=O)(=O)CCOC2] |
| 247 | 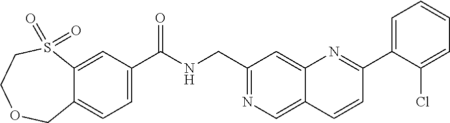 [SMILES: O=C(NCc1cc2nc(-c3ccccc3Cl)ccc2cn1)c1ccc2c(c1)S(=O)(=O)CCOC2] |
| 248 | 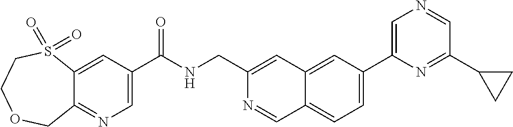 [SMILES: O=C(NCc1cc2cc(-c3cncc(C4CC4)n3)ccc2cn1)c1cnc2c(c1)S(=O)(=O)CCOC2] |
| 249 | 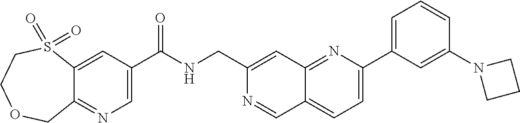 [SMILES: O=C(NCc1cc2nc(-c3cccc(N4CCC4)c3)ccc2cn1)c1cnc2c(c1)S(=O)(=O)CCOC2] |
| 250 | 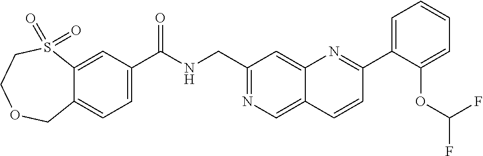 [SMILES: O=C(NCc1cc2nc(-c3ccccc3OC(F)F)ccc2cn1)c1ccc2c(c1)S(=O)(=O)CCOC2] |
| 251 | 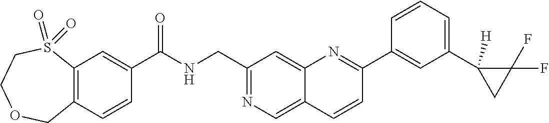 [SMILES: O=C(NCc1cc2nc(-c3cccc([C@@H]4CC4(F)F)c3)ccc2cn1)c1ccc2c(c1)S(=O)(=O)CCOC2] |
| 252 | 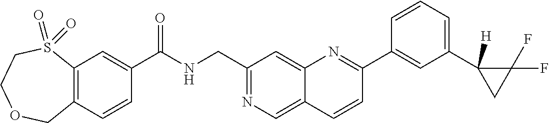 [SMILES: O=C(NCc1cc2nc(-c3cccc([C@H]4CC4(F)F)c3)ccc2cn1)c1ccc2c(c1)S(=O)(=O)CCOC2] |
| 253 | 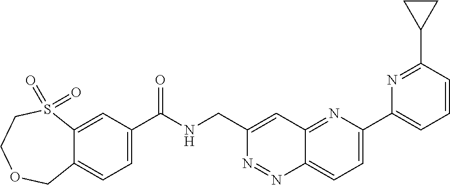 [SMILES: O=C(NCc1cc2nc(-c3cccc(C4CC4)n3)ccc2nn1)c1ccc2c(c1)S(=O)(=O)CCOC2] |
| 254 | 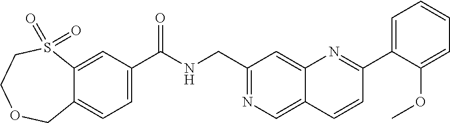 [SMILES: COc1ccccc1-c1ccc2cnc(CNC(=O)c3ccc4c(c3)S(=O)(=O)CCOC4)cc2n1] |
| 255 | 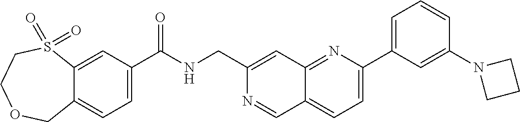 [SMILES: O=C(NCc1cc2nc(-c3cccc(N4CCC4)c3)ccc2cn1)c1ccc2c(c1)S(=O)(=O)CCOC2] |
| 256 | 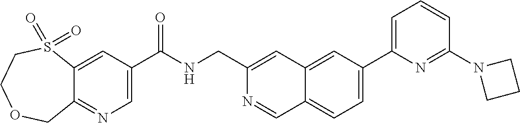 [SMILES: O=C(NCc1cc2cc(-c3cccc(N4CCC4)n3)ccc2cn1)c1cnc2c(c1)S(=O)(=O)CCOC2] |
| 257 | 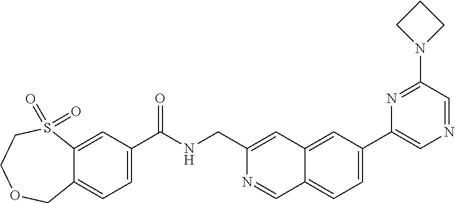 [SMILES: O=C(NCc1cc2cc(-c3cncc(N4CCC4)n3)ccc2cn1)c1ccc2c(c1)S(=O)(=O)CCOC2] |
| 258 | 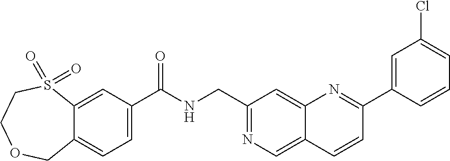 [SMILES: O=C(NCc1cc2nc(-c3cccc(Cl)c3)ccc2cn1)c1ccc2c(c1)S(=O)(=O)CCOC2] |
| 259 | 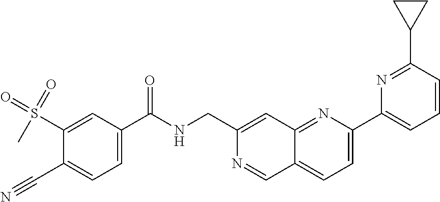 [SMILES: CS(=O)(=O)c1cc(C(=O)NCc2cc3nc(-c4cccc(C5CC5)n4)ccc3cn2)ccc1C#N] |
| 260 | 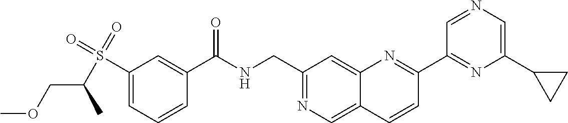 [SMILES: COC[C@H](C)S(=O)(=O)c1cccc(C(=O)NCc2cc3nc(-c4cncc(C5CC5)n4)ccc3cn2)c1] |
| 261 | 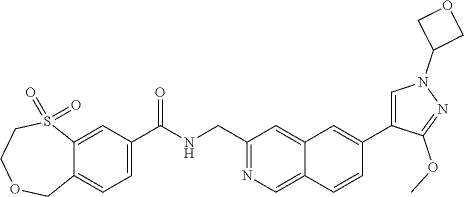 [SMILES: COc1nn(C2COC2)cc1-c1ccc2cnc(CNC(=O)c3ccc4c(c3)S(=O)(=O)CCOC4)cc2c1] |
| 262 | 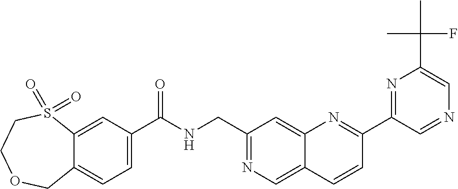 [SMILES: CC(C)(F)c1cncc(-c2ccc3cnc(CNC(=O)c4ccc5c(c4)S(=O)(=O)CCOC5)cc3n2)n1] |
| 263 | 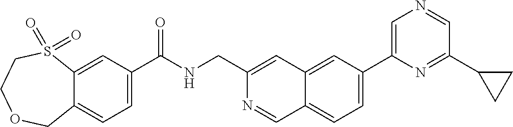 [SMILES: O=C(NCc1cc2cc(-c3cncc(C4CC4)n3)ccc2cn1)c1ccc2c(c1)S(=O)(=O)CCOC2] |
| 264 | 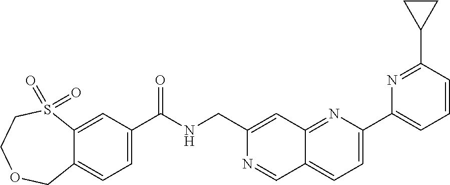 [SMILES: O=C(NCc1cc2nc(-c3cccc(C4CC4)n3)ccc2cn1)c1ccc2c(c1)S(=O)(=O)CCOC2] |
| 265 | 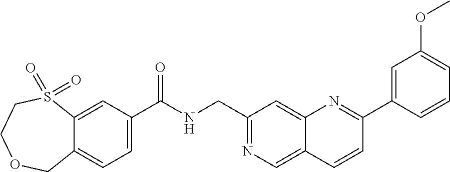 [SMILES: COc1cccc(-c2ccc3cnc(CNC(=O)c4ccc5c(c4)S(=O)(=O)CCOC5)cc3n2)c1] |
| 266 | 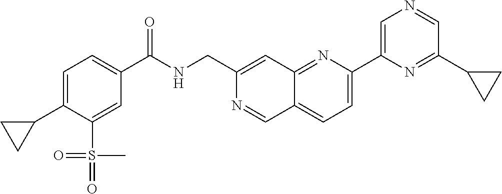 [SMILES: CS(=O)(=O)c1cc(C(=O)NCc2cc3nc(-c4cncc(C5CC5)n4)ccc3cn2)ccc1C1CC1] |
| 267 | 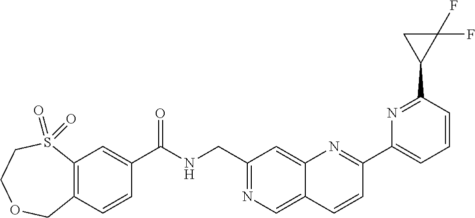 [SMILES: O=C(NCc1cc2nc(-c3cccc([C@H]4CC4(F)F)n3)ccc2cn1)c1ccc2c(c1)S(=O)(=O)CCOC2] |
| 268 | 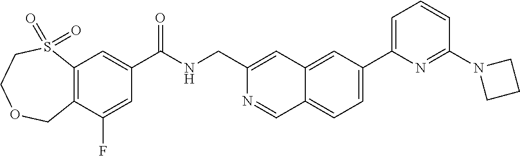 [SMILES: O=C(NCc1cc2cc(-c3cccc(N4CCC4)n3)ccc2cn1)c1cc(F)c2c(c1)S(=O)(=O)CCOC2] |
| 269 | 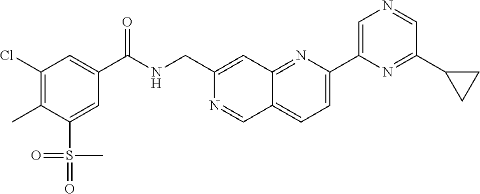 [SMILES: Cc1c(Cl)cc(C(=O)NCc2cc3nc(-c4cncc(C5CC5)n4)ccc3cn2)cc1S(C)(=O)=O] |
| 270 | 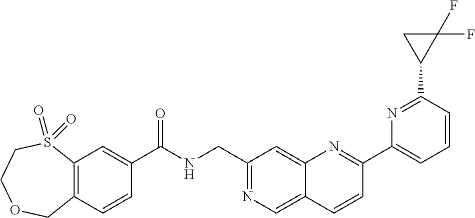 [SMILES: O=C(NCc1cc2nc(-c3cccc([C@@H]4CC4(F)F)n3)ccc2cn1)c1ccc2c(c1)S(=O)(=O)CCOC2] |
| 271 | 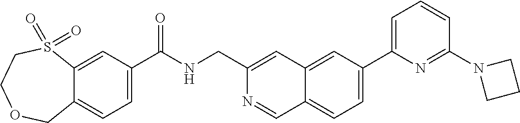 [SMILES: O=C(NCc1cc2cc(-c3cccc(N4CCC4)n3)ccc2cn1)c1ccc2c(c1)S(=O)(=O)CCOC2] |
| 272 | 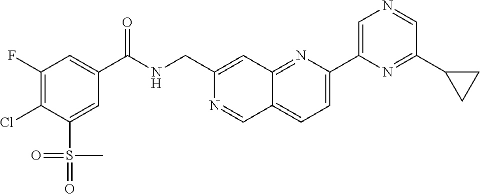 [SMILES: CS(=O)(=O)c1cc(C(=O)NCc2cc3nc(-c4cncc(C5CC5)n4)ccc3cn2)cc(F)c1Cl] |
| 273 | 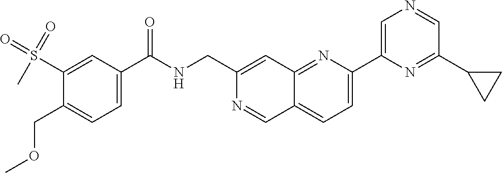 [SMILES: COCc1ccc(C(=O)NCc2cc3nc(-c4cncc(C5CC5)n4)ccc3cn2)cc1S(C)(=O)=O] |
| 274 | 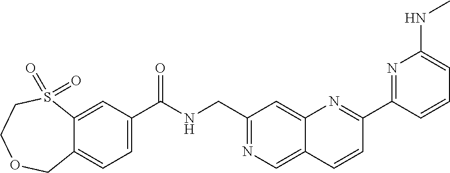 [SMILES: CNc1cccc(-c2ccc3cnc(CNC(=O)c4ccc5c(c4)S(=O)(=O)CCOC5)cc3n2)n1] |
| 275 | 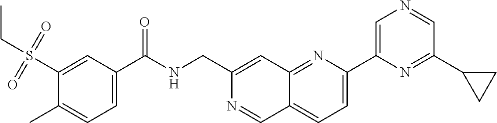 [SMILES: CCS(=O)(=O)c1cc(C(=O)NCc2cc3nc(-c4cncc(C5CC5)n4)ccc3cn2)ccc1C] |
| 276 | 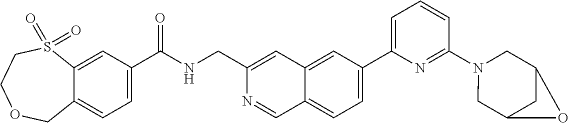 [SMILES: O=C(NCc1cc2cc(-c3cccc(N4CC5CC(C4)O5)n3)ccc2cn1)c1ccc2c(c1)S(=O)(=O)CCOC2] |
| 277 | 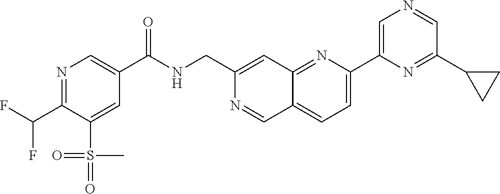 [SMILES: CS(=O)(=O)c1cc(C(=O)NCc2cc3nc(-c4cncc(C5CC5)n4)ccc3cn2)cnc1C(F)F] |
| 278 | 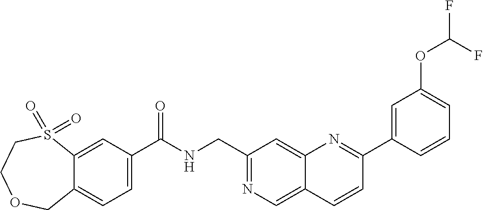 [SMILES: O=C(NCc1cc2nc(-c3cccc(OC(F)F)c3)ccc2cn1)c1ccc2c(c1)S(=O)(=O)CCOC2] |
| 279 | 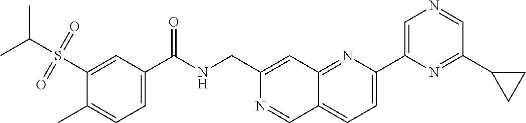 [SMILES: Cc1ccc(C(=O)NCc2cc3nc(-c4cncc(C5CC5)n4)ccc3cn2)cc1S(=O)(=O)C(C)C] |
| 280 | 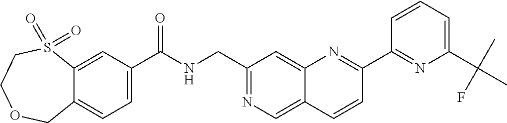 [SMILES: CC(C)(F)c1cccc(-c2ccc3cnc(CNC(=O)c4ccc5c(c4)S(=O)(=O)CCOC5)cc3n2)n1] |
| 281 | 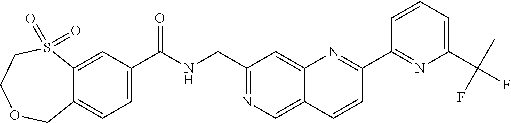 [SMILES: CC(F)(F)c1cccc(-c2ccc3cnc(CNC(=O)c4ccc5c(c4)S(=O)(=O)CCOC5)cc3n2)n1] |
| 282 | 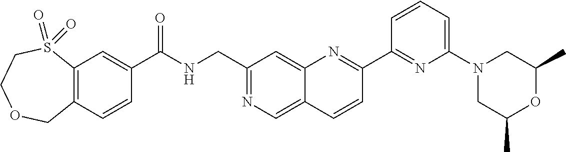 [SMILES: C[C@@H]1CN(c2cccc(-c3ccc4cnc(CNC(=O)c5ccc6c(c5)S(=O)(=O)CCOC6)cc4n3)n2)C[C@H](C)O1] |
| 283 | 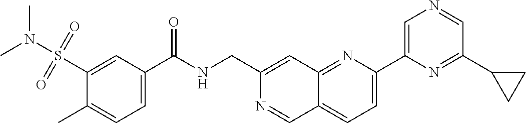 [SMILES: Cc1ccc(C(=O)NCc2cc3nc(-c4cncc(C5CC5)n4)ccc3cn2)cc1S(=O)(=O)N(C)C] |
| 284 | 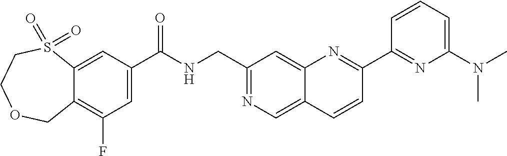 [SMILES: CN(C)c1cccc(-c2ccc3cnc(CNC(=O)c4cc(F)c5c(c4)S(=O)(=O)CCOC5)cc3n2)n1] |
| 285 | 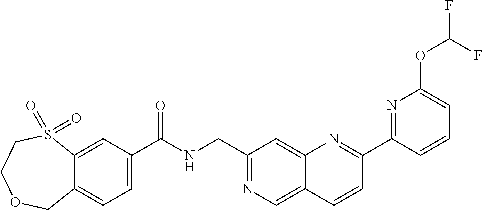 [SMILES: O=C(NCc1cc2nc(-c3cccc(OC(F)F)n3)ccc2cn1)c1ccc2c(c1)S(=O)(=O)CCOC2] |
| 286 | 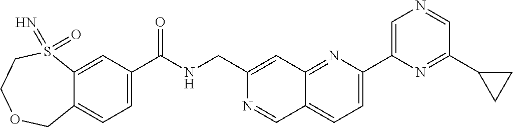 [SMILES: N=S1(=O)CCOCc2ccc(C(=O)NCc3cc4nc(-c5cncc(C6CC6)n5)ccc4cn3)cc21] |
| 287 | 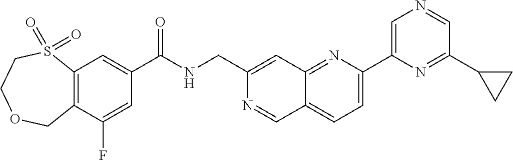 [SMILES: O=C(NCc1cc2nc(-c3cncc(C4CC4)n3)ccc2cn1)c1cc(F)c2c(c1)S(=O)(=O)CCOC2] |
| 288 | 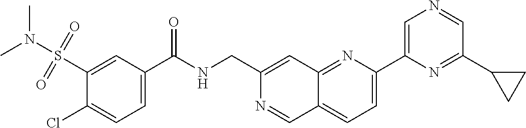 [SMILES: CN(C)S(=O)(=O)c1cc(C(=O)NCc2cc3nc(-c4cncc(C5CC5)n4)ccc3cn2)ccc1Cl] |
| 289 | 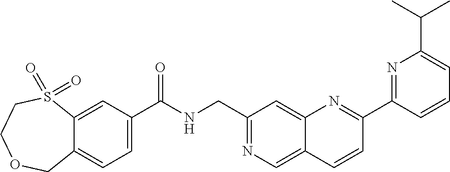 [SMILES: CC(C)c1cccc(-c2ccc3cnc(CNC(=O)c4ccc5c(c4)S(=O)(=O)CCOC5)cc3n2)n1] |
| 290 | 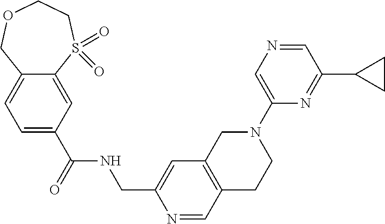 [SMILES: O=C(NCc1cc2c(cn1)CCN(c1cncc(C3CC3)n1)C2)c1ccc2c(c1)S(=O)(=O)CCOC2] |
| 291 | 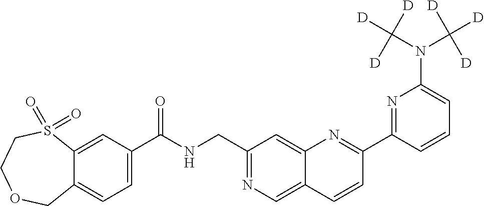 [SMILES: [2H]C([2H])([2H])N(c1cccc(-c2ccc3cnc(CNC(=O)c4ccc5c(c4)S(=O)(=O)CCOC5)cc3n2)n1)C([2H])([2H])[2H]] |
| 292 | 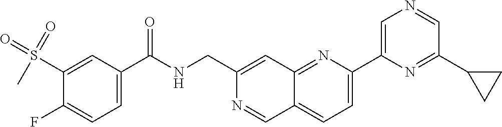 [SMILES: CS(=O)(=O)c1cc(C(=O)NCc2cc3nc(-c4cncc(C5CC5)n4)ccc3cn2)ccc1F] |
| 293 | 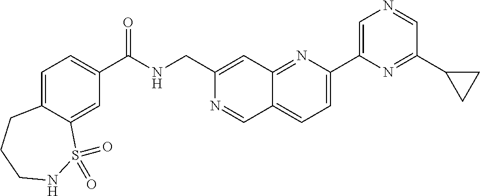 [SMILES: O=C(NCc1cc2nc(-c3cncc(C4CC4)n3)ccc2cn1)c1ccc2c(c1)S(=O)(=O)NCCC2] |
| 294 | 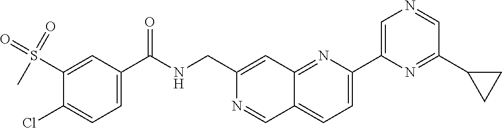 [SMILES: CS(=O)(=O)c1cc(C(=O)NCc2cc3nc(-c4cncc(C5CC5)n4)ccc3cn2)ccc1Cl] |
| 295 | 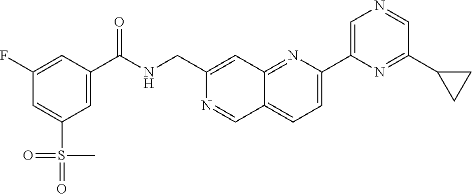 [SMILES: CS(=O)(=O)c1cc(F)cc(C(=O)NCc2cc3nc(-c4cncc(C5CC5)n4)ccc3cn2)c1] |
| 296 | 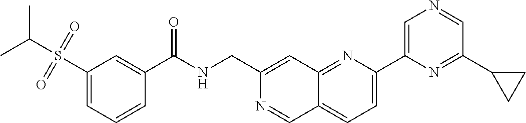 [SMILES: CC(C)S(=O)(=O)c1cccc(C(=O)NCc2cc3nc(-c4cncc(C5CC5)n4)ccc3cn2)c1] |
| 297 | 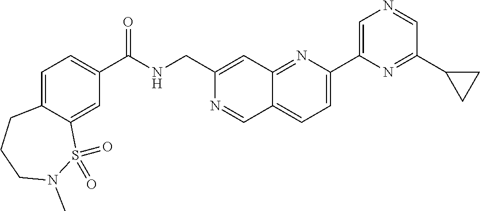 [SMILES: CN1CCCc2ccc(C(=O)NCc3cc4nc(-c5cncc(C6CC6)n5)ccc4cn3)cc2S1(=O)=O] |
| 298 | 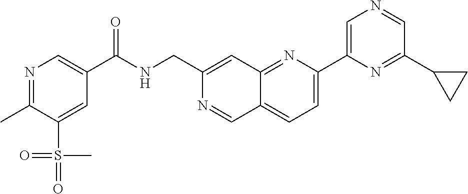 [SMILES: Cc1ncc(C(=O)NCc2cc3nc(-c4cncc(C5CC5)n4)ccc3cn2)cc1S(C)(=O)=O] |
| 299 | 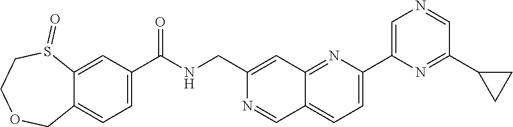 [SMILES: O=C(NCc1cc2nc(-c3cncc(C4CC4)n3)ccc2cn1)c1ccc2c(c1)S(=O)CCOC2] |
| 300 | 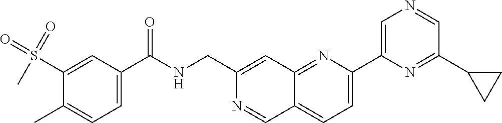 [SMILES: Cc1ccc(C(=O)NCc2cc3nc(-c4cncc(C5CC5)n4)ccc3cn2)cc1S(C)(=O)=O] |
| 301 | 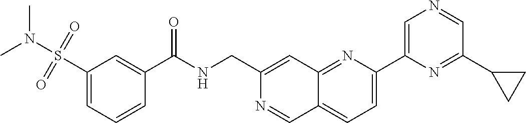 [SMILES: CN(C)S(=O)(=O)c1cccc(C(=O)NCc2cc3nc(-c4cncc(C5CC5)n4)ccc3cn2)c1] |
| 302 | 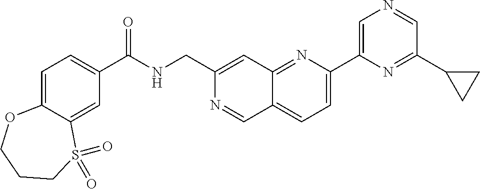 [SMILES: O=C(NCc1cc2nc(-c3cncc(C4CC4)n3)ccc2cn1)c1ccc2c(c1)S(=O)(=O)CCCO2] |
| 303 | 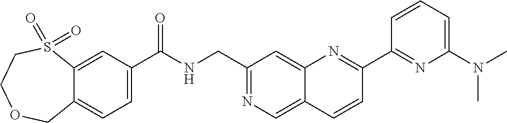 [SMILES: CN(C)c1cccc(-c2ccc3cnc(CNC(=O)c4ccc5c(c4)S(=O)(=O)CCOC5)cc3n2)n1] |
| 304 | 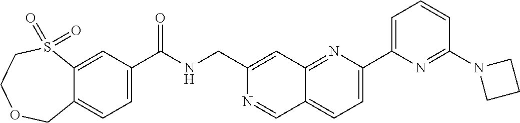 [SMILES: O=C(NCc1cc2nc(-c3cccc(N4CCC4)n3)ccc2cn1)c1ccc2c(c1)S(=O)(=O)CCOC2] |
| 305 | 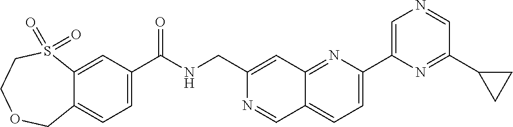 [SMILES: O=C(NCc1cc2nc(-c3cncc(C4CC4)n3)ccc2cn1)c1ccc2c(c1)S(=O)(=O)CCOC2] |
| 306 | 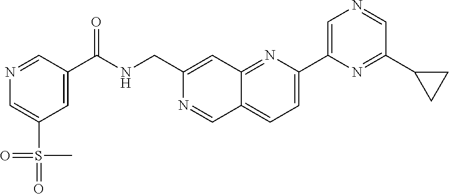 [SMILES: CS(=O)(=O)c1cncc(C(=O)NCc2cc3nc(-c4cncc(C5CC5)n4)ccc3cn2)c1] |
| 307 | 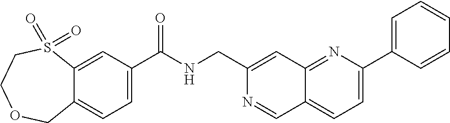 [SMILES: O=C(NCc1cc2nc(-c3ccccc3)ccc2cn1)c1ccc2c(c1)S(=O)(=O)CCOC2] |
| 308 | 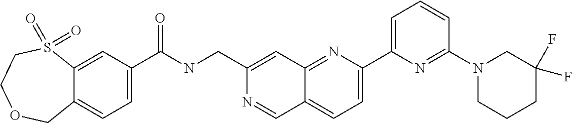 [SMILES: O=C(NCc1cc2nc(-c3cccc(N4CCCC(F)(F)C4)n3)ccc2cn1)c1ccc2c(c1)S(=O)(=O)CCOC2] |

In another aspect, the invention provides a compound selected from the group consisting of compounds 309-856 in Table 1B and pharmaceutically acceptable salts thereof.

TABLE 1B
Compounds of the invention (cyclic and acyclic sulfones)

| Compound | |
|---|---|
| # | Structure |
| 309 | 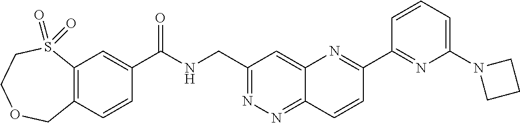 [SMILES: O=C(NCc1cc2nc(-c3cccc(N4CCC4)n3)ccc2nn1)c1ccc2c(c1)S(=O)(=O)CCOC2] |
| 310 | 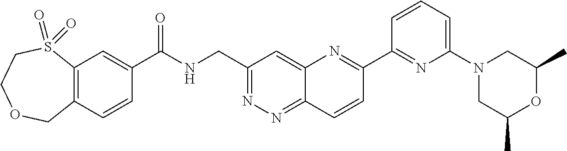 [SMILES: C[C@@H]1CN(c2cccc(-c3ccc4nnc(CNC(=O)c5ccc6c(c5)S(=O)(=O)CCOC6)cc4n3)n2)C[C@H](C)O1] |
| 311 | 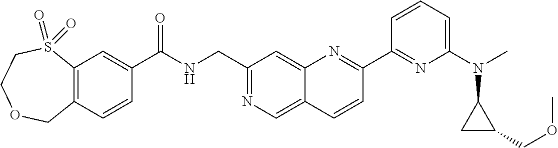 [SMILES: COC[C@@H]1C[C@H]1N(C)c1cccc(-c2ccc3cnc(CNC(=O)c4ccc5c(c4)S(=O)(=O)CCOC5)cc3n2)n1] |
| 312 | 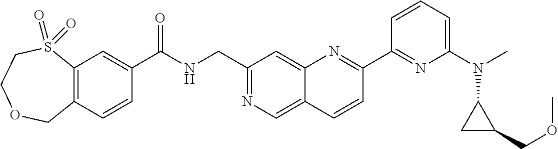 [SMILES: COC[C@H]1C[C@@H]1N(C)c1cccc(-c2ccc3cnc(CNC(=O)c4ccc5c(c4)S(=O)(=O)CCOC5)cc3n2)n1] |
| 313 | 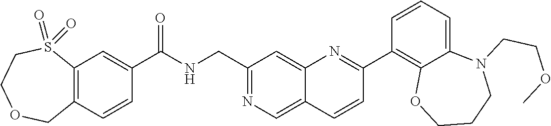 [SMILES: COCCN1CCCOc2c(-c3ccc4cnc(CNC(=O)c5ccc6c(c5)S(=O)(=O)CCOC6)cc4n3)cccc21] |
| 314 | 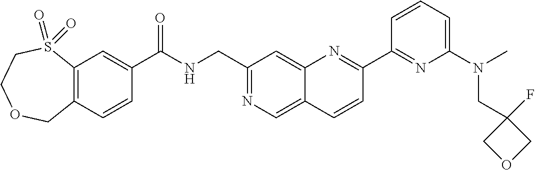 [SMILES: CN(CC1(F)COC1)c1cccc(-c2ccc3cnc(CNC(=O)c4ccc5c(c4)S(=O)(=O)CCOC5)cc3n2)n1] |
| 315 | 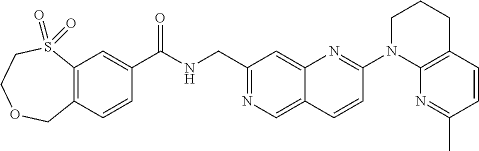 [SMILES: Cc1ccc2c(n1)N(c1ccc3cnc(CNC(=O)c4ccc5c(c4)S(=O)(=O)CCOC5)cc3n1)CCC2] |
| 316 | 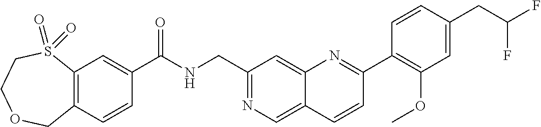 [SMILES: COc1cc(CC(F)F)ccc1-c1ccc2cnc(CNC(=O)c3ccc4c(c3)S(=O)(=O)CCOC4)cc2n1] |
| 317 | 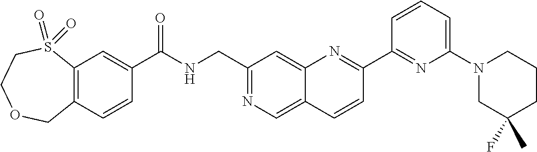 [SMILES: C[C@]1(F)CCCN(c2cccc(-c3ccc4cnc(CNC(=O)c5ccc6c(c5)S(=O)(=O)CCOC6)cc4n3)n2)C1] |
| 318 | 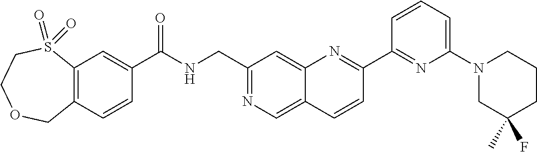 [SMILES: C[C@@]1(F)CCCN(c2cccc(-c3ccc4cnc(CNC(=O)c5ccc6c(c5)S(=O)(=O)CCOC6)cc4n3)n2)C1] |
| 319 | 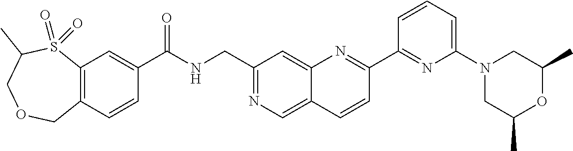 |
| 320 | 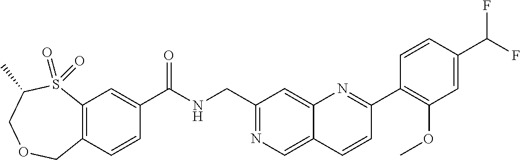 [SMILES: COc1cc(C(F)F)ccc1-c1ccc2cnc(CNC(=O)c3ccc4c(c3)S(=O)(=O)[C@@H](C)COC4)cc2n1] |
| 321 | 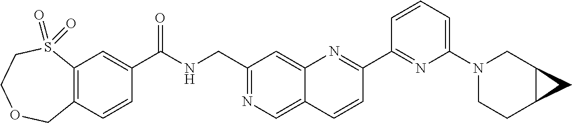 [SMILES: O=C(NCc1cc2nc(-c3cccc(N4CC[C@@H]5C[C@@H]5C4)n3)ccc2cn1)c1ccc2c(c1)S(=O)(=O)CCOC2] |
| 322 | 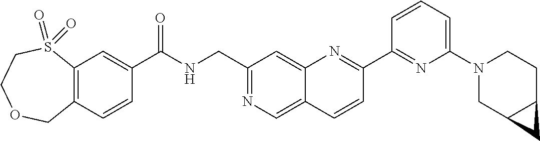 [SMILES: O=C(NCc1cc2nc(-c3cccc(N4CC[C@H]5C[C@H]5C4)n3)ccc2cn1)c1ccc2c(c1)S(=O)(=O)CCOC2] |
| 323 | 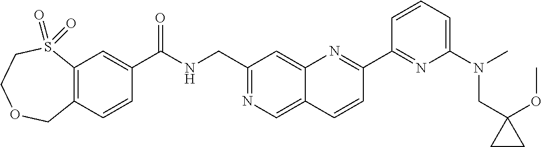 [SMILES: COC1(CN(C)c2cccc(-c3ccc4cnc(CNC(=O)c5ccc6c(c5)S(=O)(=O)CCOC6)cc4n3)n2)CC1] |
| 324 | 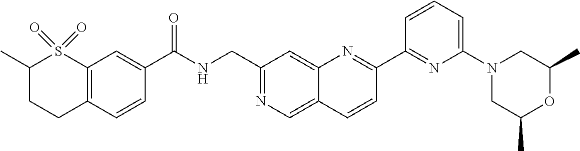 |
| 325 | 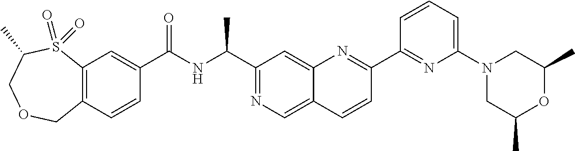 |
| 326 | 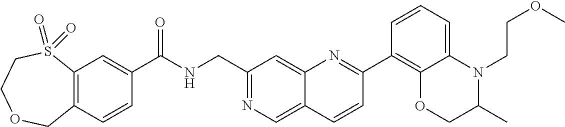 [SMILES: COCCN1c2cccc(-c3ccc4cnc(CNC(=O)c5ccc6c(c5)S(=O)(=O)CCOC6)cc4n3)c2OCC1C] |
| 327 | 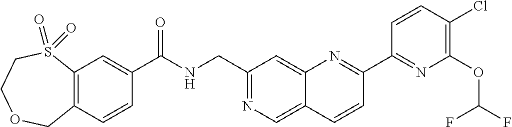 [SMILES: O=C(NCc1cc2nc(-c3ccc(Cl)c(OC(F)F)n3)ccc2cn1)c1ccc2c(c1)S(=O)(=O)CCOC2] |
| 328 | 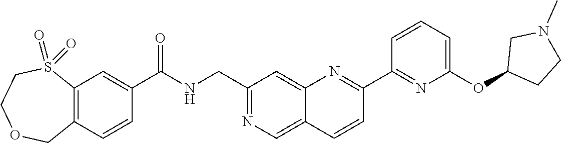 [SMILES: CN1CC[C@@H](Oc2cccc(-c3ccc4cnc(CNC(=O)c5ccc6c(c5)S(=O)(=O)CCOC6)cc4n3)n2)C1] |
| 329 | 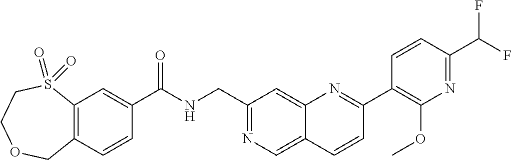 [SMILES: COc1nc(C(F)F)ccc1-c1ccc2cnc(CNC(=O)c3ccc4c(c3)S(=O)(=O)CCOC4)cc2n1] |
| 330 | 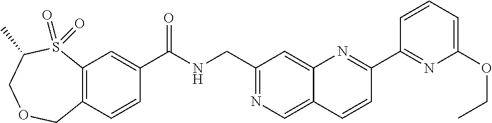 [SMILES: CCOc1cccc(-c2ccc3cnc(CNC(=O)c4ccc5c(c4)S(=O)(=O)[C@@H](C)COC5)cc3n2)n1] |
| 331 | 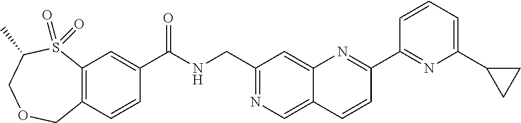 [SMILES: C[C@H]1COCc2ccc(C(=O)NCc3cc4nc(-c5cccc(C6CC6)n5)ccc4cn3)cc2S1(=O)=O] |
| 332 | 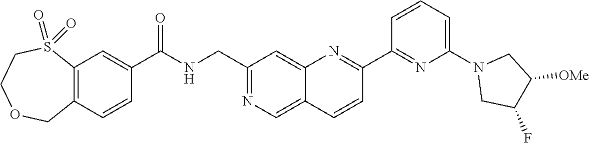 [SMILES: CO[C@H]1CN(c2cccc(-c3ccc4cnc(CNC(=O)c5ccc6c(c5)S(=O)(=O)CCOC6)cc4n3)n2)C[C@H]1F] |
| 333 | 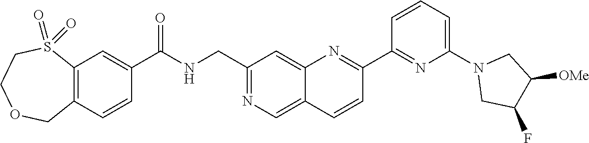 [SMILES: CO[C@@H]1CN(c2cccc(-c3ccc4cnc(CNC(=O)c5ccc6c(c5)S(=O)(=O)CCOC6)cc4n3)n2)C[C@@H]1F] |
| 334 | 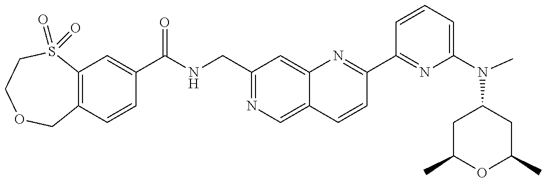 [SMILES: C[C@@H]1C[C@@H](N(C)c2cccc(-c3ccc4cnc(CNC(=O)c5ccc6c(c5)S(=O)(=O)CCOC6)cc4n3)n2)C[C@H](C)O1] |
| 335 | 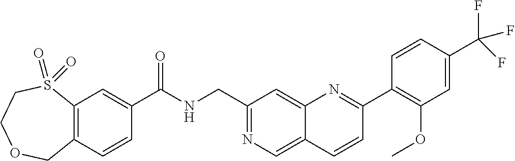 [SMILES: COc1cc(C(F)(F)F)ccc1-c1ccc2cnc(CNC(=O)c3ccc4c(c3)S(=O)(=O)CCOC4)cc2n1] |
| 336 | 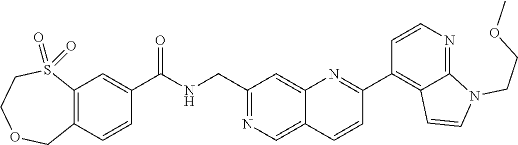 [SMILES: COCCn1ccc2c(-c3ccc4cnc(CNC(=O)c5ccc6c(c5)S(=O)(=O)CCOC6)cc4n3)ccnc21] |
| 337 | 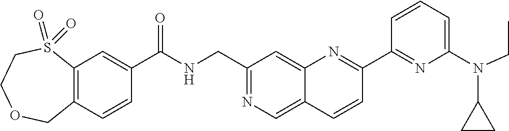 [SMILES: CCN(c1cccc(-c2ccc3cnc(CNC(=O)c4ccc5c(c4)S(=O)(=O)CCOC5)cc3n2)n1)C1CC1] |
| 338 | 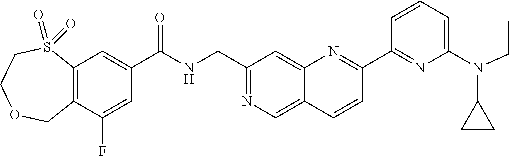 [SMILES: CCN(c1cccc(-c2ccc3cnc(CNC(=O)c4cc(F)c5c(c4)S(=O)(=O)CCOC5)cc3n2)n1)C1CC1] |
| 339 | 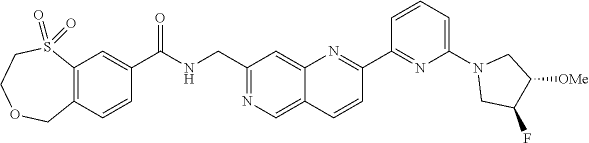 [SMILES: CO[C@H]1CN(c2cccc(-c3ccc4cnc(CNC(=O)c5ccc6c(c5)S(=O)(=O)CCOC6)cc4n3)n2)C[C@@H]1F] |
| 340 | 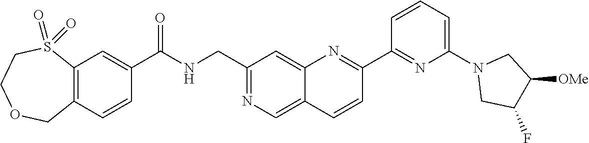 [SMILES: CO[C@@H]1CN(c2cccc(-c3ccc4cnc(CNC(=O)c5ccc6c(c5)S(=O)(=O)CCOC6)cc4n3)n2)C[C@H]1F] |
| 341 | 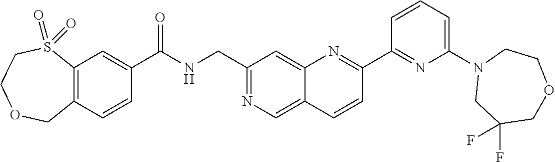 [SMILES: O=C(NCc1cc2nc(-c3cccc(N4CCOCC(F)(F)C4)n3)ccc2cn1)c1ccc2c(c1)S(=O)(=O)CCOC2] |
| 342 | 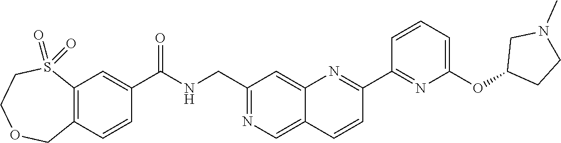 [SMILES: CN1CC[C@H](Oc2cccc(-c3ccc4cnc(CNC(=O)c5ccc6c(c5)S(=O)(=O)CCOC6)cc4n3)n2)C1] |
| 343 | 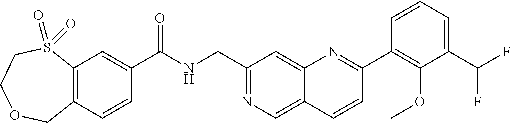 [SMILES: COc1c(-c2ccc3cnc(CNC(=O)c4ccc5c(c4)S(=O)(=O)CCOC5)cc3n2)cccc1C(F)F] |
| 344 | 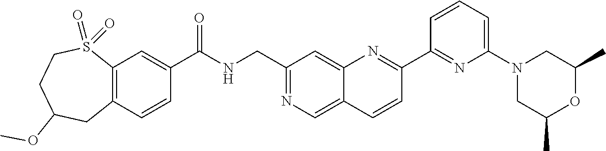 |
| 345 | 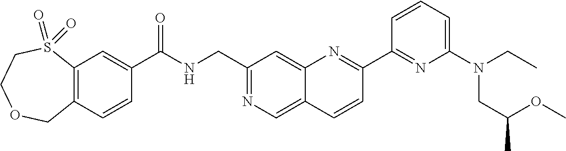 [SMILES: CCN(C[C@H](C)OC)c1cccc(-c2ccc3cnc(CNC(=O)c4ccc5c(c4)S(=O)(=O)CCOC5)cc3n2)n1] |
| 346 | 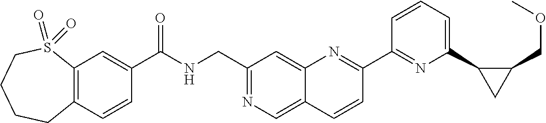 [SMILES: COC[C@H]1C[C@H]1c1cccc(-c2ccc3cnc(CNC(=O)c4ccc5c(c4)S(=O)(=O)CCCC5)cc3n2)n1] |
| 347 | 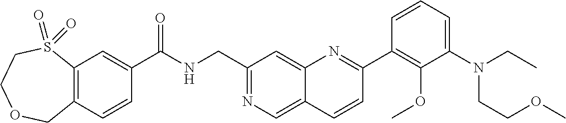 [SMILES: CCN(CCOC)c1cccc(-c2ccc3cnc(CNC(=O)c4ccc5c(c4)S(=O)(=O)CCOC5)cc3n2)c1OC] |
| 348 | 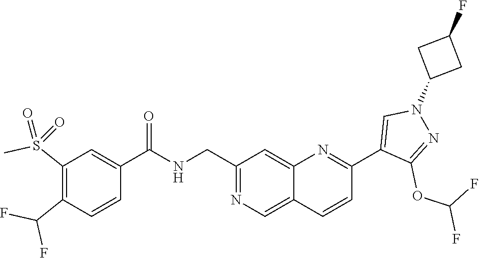 [SMILES: CS(=O)(=O)c1cc(C(=O)NCc2cc3nc(-c4cn([C@H]5C[C@H](F)C5)nc4OC(F)F)ccc3cn2)ccc1C(F)F] |
| 349 | 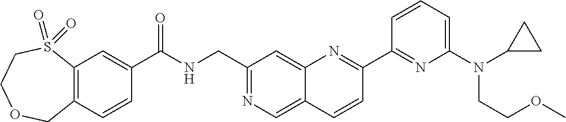 [SMILES: COCCN(c1cccc(-c2ccc3cnc(CNC(=O)c4ccc5c(c4)S(=O)(=O)CCOC5)cc3n2)n1)C1CC1] |
| 350 | 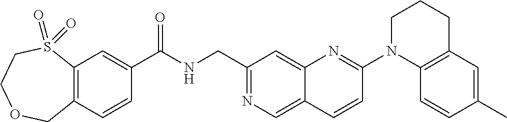 [SMILES: Cc1ccc2c(c1)CCCN2c1ccc2cnc(CNC(=O)c3ccc4c(c3)S(=O)(=O)CCOC4)cc2n1] |
| 351 | 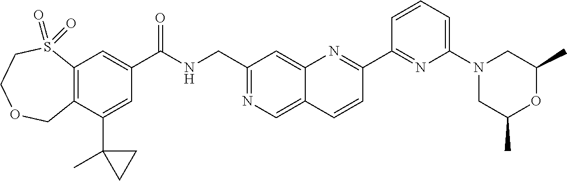 [SMILES: C[C@@H]1CN(c2cccc(-c3ccc4cnc(CNC(=O)c5cc(C6(C)CC6)c6c(c5)S(=O)(=O)CCOC6)cc4n3)n2)C[C@H](C)O1] |
| 352 | 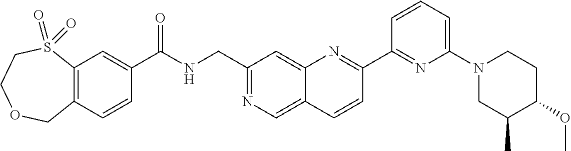 [SMILES: CO[C@H]1CCN(c2cccc(-c3ccc4cnc(CNC(=O)c5ccc6c(c5)S(=O)(=O)CCOC6)cc4n3)n2)C[C@@H]1C] |
| 353 | 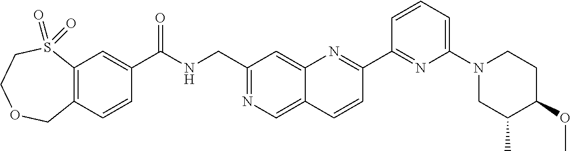 [SMILES: CO[C@@H]1CCN(c2cccc(-c3ccc4cnc(CNC(=O)c5ccc6c(c5)S(=O)(=O)CCOC6)cc4n3)n2)C[C@H]1C] |
| 354 | 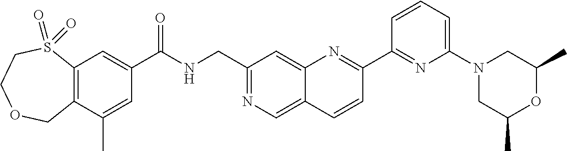 [SMILES: Cc1cc(C(=O)NCc2cc3nc(-c4cccc(N5C[C@@H](C)O[C@@H](C)C5)n4)ccc3cn2)cc2c1COCCS2(=O)=O] |
| 355 | 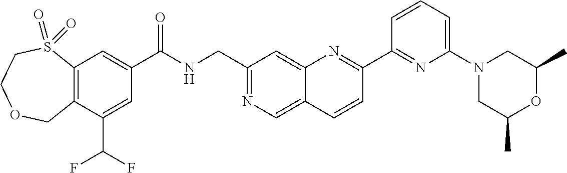 [SMILES: C[C@@H]1CN(c2cccc(-c3ccc4cnc(CNC(=O)c5cc(C(F)F)c6c(c5)S(=O)(=O)CCOC6)cc4n3)n2)C[C@H](C)O1] |
| 356 | 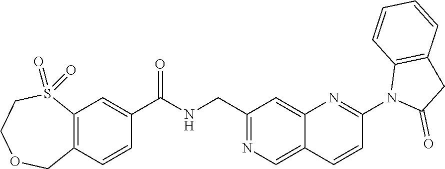 [SMILES: O=C(NCc1cc2nc(N3C(=O)Cc4ccccc43)ccc2cn1)c1ccc2c(c1)S(=O)(=O)CCOC2] |
| 357 | 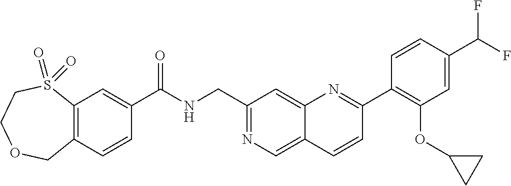 [SMILES: O=C(NCc1cc2nc(-c3ccc(C(F)F)cc3OC3CC3)ccc2cn1)c1ccc2c(c1)S(=O)(=O)CCOC2] |
| 358 | 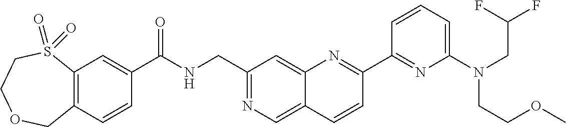 [SMILES: COCCN(CC(F)F)c1cccc(-c2ccc3cnc(CNC(=O)c4ccc5c(c4)S(=O)(=O)CCOC5)cc3n2)n1] |
| 359 | 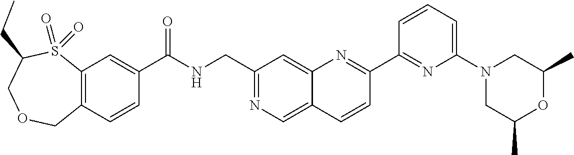 [SMILES: CC[C@@H]1COCc2ccc(C(=O)NCc3cc4nc(-c5cccc(N6C[C@@H](C)O[C@@H](C)C6)n5)ccc4cn3)cc2S1(=O)=O] |
| 360 | 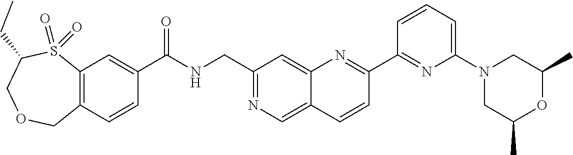 [SMILES: CC[C@H]1COCc2ccc(C(=O)NCc3cc4nc(-c5cccc(N6C[C@@H](C)O[C@@H](C)C6)n5)ccc4cn3)cc2S1(=O)=O] |
| 361 | 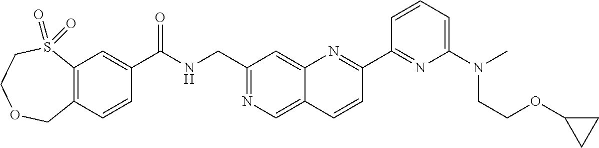 [SMILES: CN(CCOC1CC1)c1cccc(-c2ccc3cnc(CNC(=O)c4ccc5c(c4)S(=O)(=O)CCOC5)cc3n2)n1] |
| 362 | 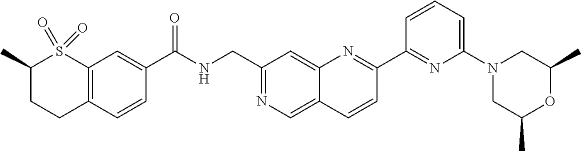 |
| 363 | 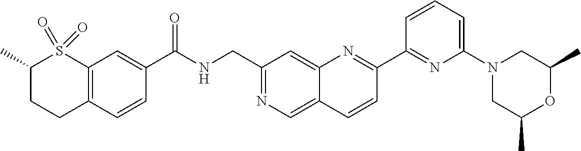 |
| 364 | 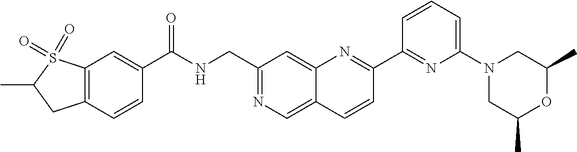 |
| 365 | 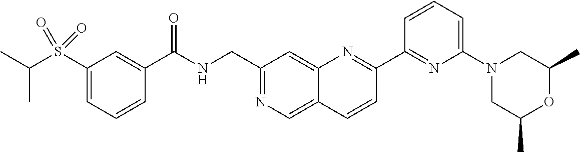 [SMILES: CC(C)S(=O)(=O)c1cccc(C(=O)NCc2cc3nc(-c4cccc(N5C[C@@H](C)O[C@@H](C)C5)n4)ccc3cn2)c1] |
| 366 | 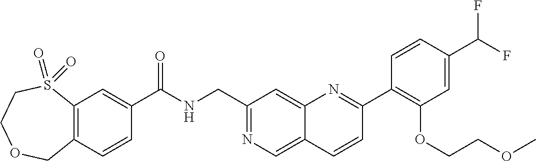 [SMILES: COCCOc1cc(C(F)F)ccc1-c1ccc2cnc(CNC(=O)c3ccc4c(c3)S(=O)(=O)CCOC4)cc2n1] |
| 367 | 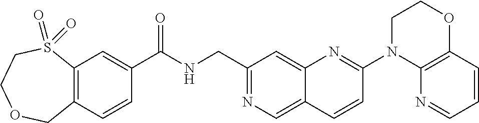 [SMILES: O=C(NCc1cc2nc(N3CCOc4cccnc43)ccc2cn1)c1ccc2c(c1)S(=O)(=O)CCOC2] |
| 368 | 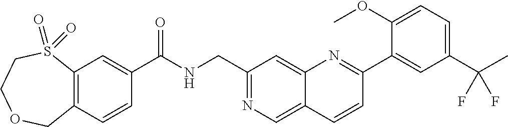 [SMILES: COc1ccc(C(C)(F)F)cc1-c1ccc2cnc(CNC(=O)c3ccc4c(c3)S(=O)(=O)CCOC4)cc2n1] |
| 369 | 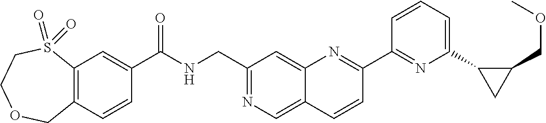 [SMILES: COC[C@H]1C[C@@H]1c1cccc(-c2ccc3cnc(CNC(=O)c4ccc5c(c4)S(=O)(=O)CCOC5)cc3n2)n1] |
| 370 | 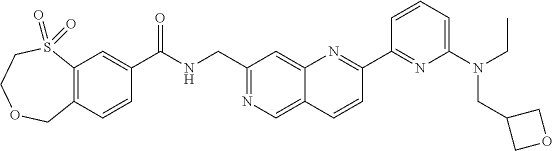 [SMILES: CCN(CC1COC1)c1cccc(-c2ccc3cnc(CNC(=O)c4ccc5c(c4)S(=O)(=O)CCOC5)cc3n2)n1] |
| 371 | 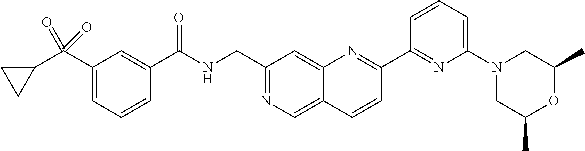 |
| 372 | 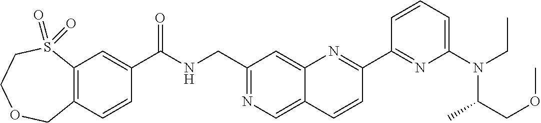 [SMILES: CCN(c1cccc(-c2ccc3cnc(CNC(=O)c4ccc5c(c4)S(=O)(=O)CCOC5)cc3n2)n1)[C@@H](C)COC] |
| 373 | 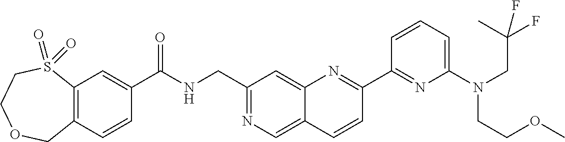 [SMILES: COCCN(CC(C)(F)F)c1cccc(-c2ccc3cnc(CNC(=O)c4ccc5c(c4)S(=O)(=O)CCOC5)cc3n2)n1] |
| 374 | 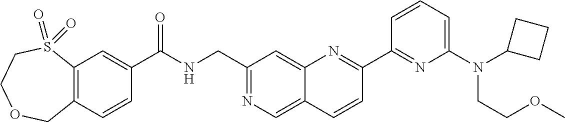 [SMILES: COCCN(c1cccc(-c2ccc3cnc(CNC(=O)c4ccc5c(c4)S(=O)(=O)CCOC5)cc3n2)n1)C1CCC1] |
| 375 | 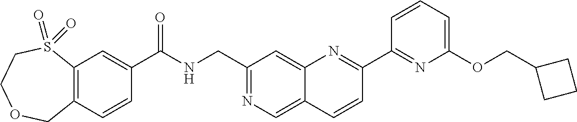 [SMILES: O=C(NCc1cc2nc(-c3cccc(OCC4CCC4)n3)ccc2cn1)c1ccc2c(c1)S(=O)(=O)CCOC2] |
| 376 | 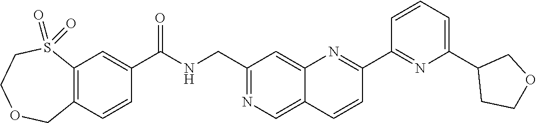 [SMILES: O=C(NCc1cc2nc(-c3cccc(C4CCOC4)n3)ccc2cn1)c1ccc2c(c1)S(=O)(=O)CCOC2] |
| 377 | 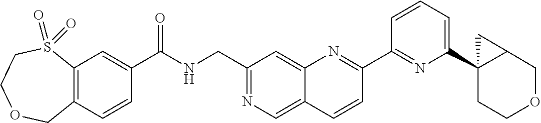 |
| 378 | 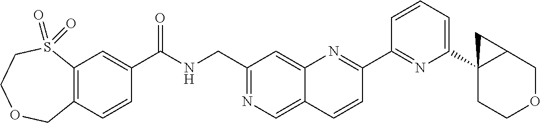 |
| 379 | 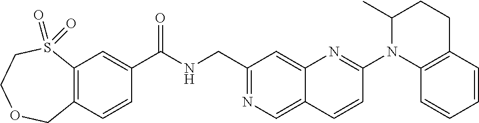 [SMILES: CC1CCc2ccccc2N1c1ccc2cnc(CNC(=O)c3ccc4c(c3)S(=O)(=O)CCOC4)cc2n1] |
| 380 | 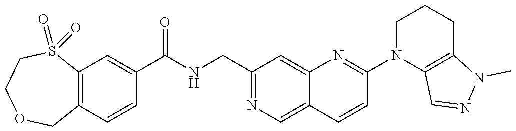 [SMILES: Cn1ncc2c1CCCN2c1ccc2cnc(CNC(=O)c3ccc4c(c3)S(=O)(=O)CCOC4)cc2n1] |
| 381 | 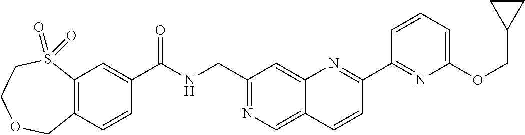 [SMILES: O=C(NCc1cc2nc(-c3cccc(OCC4CC4)n3)ccc2cn1)c1ccc2c(c1)S(=O)(=O)CCOC2] |
| 382 | 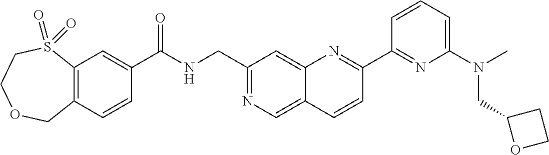 [SMILES: CN(C[C@@H]1CCO1)c1cccc(-c2ccc3cnc(CNC(=O)c4ccc5c(c4)S(=O)(=O)CCOC5)cc3n2)n1] |
| 383 | 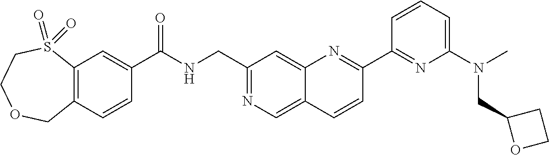 [SMILES: CN(C[C@H]1CCO1)c1cccc(-c2ccc3cnc(CNC(=O)c4ccc5c(c4)S(=O)(=O)CCOC5)cc3n2)n1] |
| 384 | 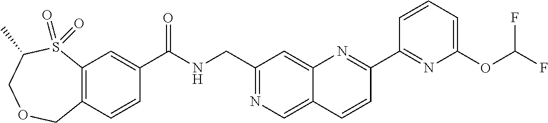 [SMILES: C[C@H]1COCc2ccc(C(=O)NCc3cc4nc(-c5cccc(OC(F)F)n5)ccc4cn3)cc2S1(=O)=O] |
| 386 | 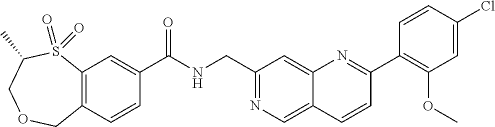 [SMILES: COc1cc(Cl)ccc1-c1ccc2cnc(CNC(=O)c3ccc4c(c3)S(=O)(=O)[C@@H](C)COC4)cc2n1] |
| 387 | 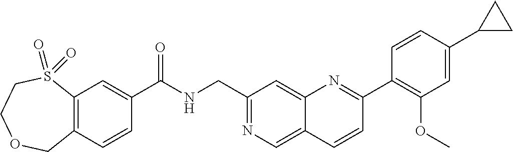 [SMILES: COc1cc(C2CC2)ccc1-c1ccc2cnc(CNC(=O)c3ccc4c(c3)S(=O)(=O)CCOC4)cc2n1] |
| 388 | 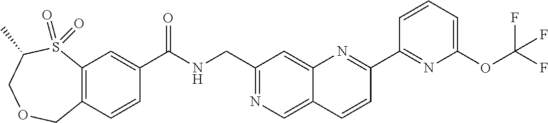 [SMILES: C[C@H]1COCc2ccc(C(=O)NCc3cc4nc(-c5cccc(OC(F)(F)F)n5)ccc4cn3)cc2S1(=O)=O] |
| 389 | 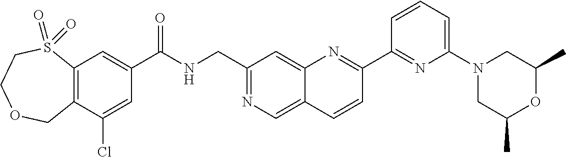 [SMILES: C[C@@H]1CN(c2cccc(-c3ccc4cnc(CNC(=O)c5cc(Cl)c6c(c5)S(=O)(=O)CCOC6)cc4n3)n2)C[C@H](C)O1] |
| 390 | 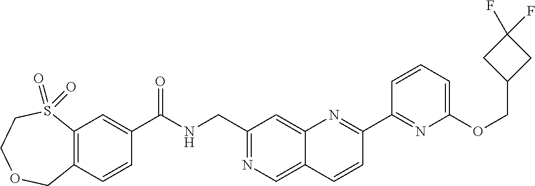 [SMILES: O=C(NCc1cc2nc(-c3cccc(OCC4CC(F)(F)C4)n3)ccc2cn1)c1ccc2c(c1)S(=O)(=O)CCOC2] |
| 391 | 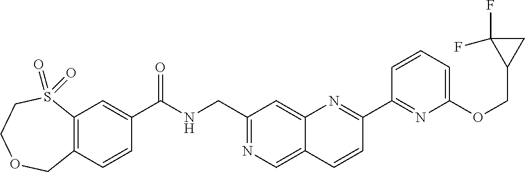 [SMILES: O=C(NCc1cc2nc(-c3cccc(OCC4CC4(F)F)n3)ccc2cn1)c1ccc2c(c1)S(=O)(=O)CCOC2] |
| 392 | 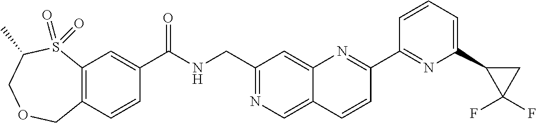 [SMILES: C[C@H]1COCc2ccc(C(=O)NCc3cc4nc(-c5cccc([C@H]6CC6(F)F)n5)ccc4cn3)cc2S1(=O)=O] |
| 393 | 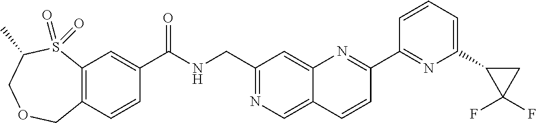 [SMILES: C[C@H]1COCc2ccc(C(=O)NCc3cc4nc(-c5cccc([C@@H]6CC6(F)F)n5)ccc4cn3)cc2S1(=O)=O] |
| 394 | 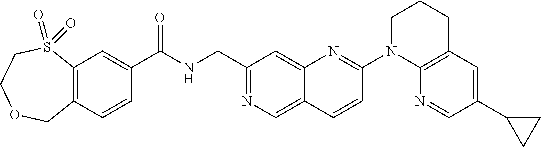 [SMILES: O=C(NCc1cc2nc(N3CCCc4cc(C5CC5)cnc43)ccc2cn1)c1ccc2c(c1)S(=O)(=O)CCOC2] |
| 395 | 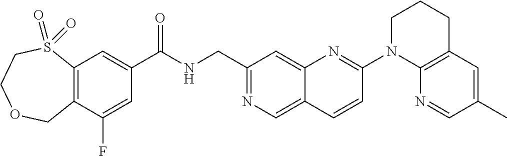 [SMILES: Cc1cnc2c(c1)CCCN2c1ccc2cnc(CNC(=O)c3cc(F)c4c(c3)S(=O)(=O)CCOC4)cc2n1] |
| 396 | 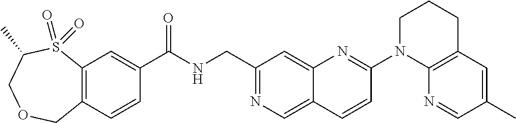 |
| 397 | 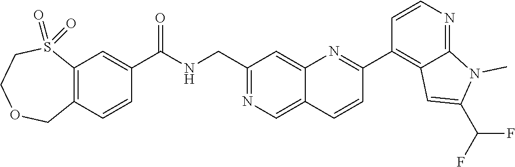 [SMILES: Cn1c(C(F)F)cc2c(-c3ccc4cnc(CNC(=O)c5ccc6c(c5)S(=O)(=O)CCOC6)cc4n3)ccnc21] |
| 398 | 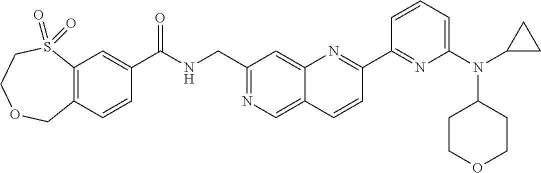 [SMILES: O=C(NCc1cc2nc(-c3cccc(N(C4CCOCC4)C4CC4)n3)ccc2cn1)c1ccc2c(c1)S(=O)(=O)CCOC2] |
| 399 | 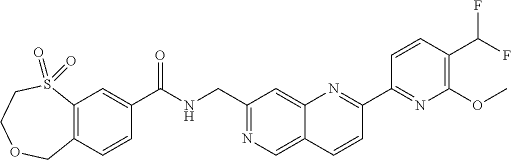 [SMILES: COc1nc(-c2ccc3cnc(CNC(=O)c4ccc5c(c4)S(=O)(=O)CCOC5)cc3n2)ccc1C(F)F] |
| 400 | 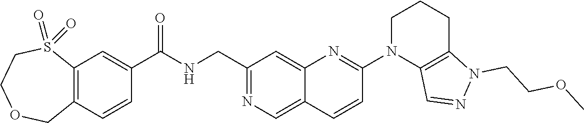 [SMILES: COCCn1ncc2c1CCCN2c1ccc2cnc(CNC(=O)c3ccc4c(c3)S(=O)(=O)CCOC4)cc2n1] |
| 401 | 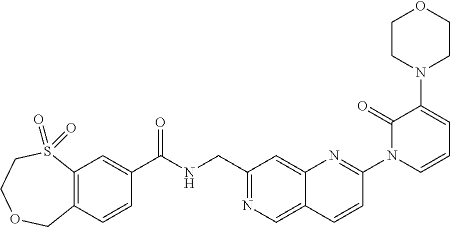 [SMILES: O=C(NCc1cc2nc(-n3cccc(N4CCOCC4)c3=O)ccc2cn1)c1ccc2c(c1)S(=O)(=O)CCOC2] |
| 402 | 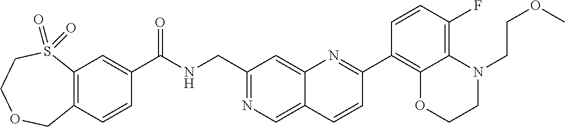 [SMILES: COCCN1CCOc2c(-c3ccc4cnc(CNC(=O)c5ccc6c(c5)S(=O)(=O)CCOC6)cc4n3)ccc(F)c21] |
| 404 | 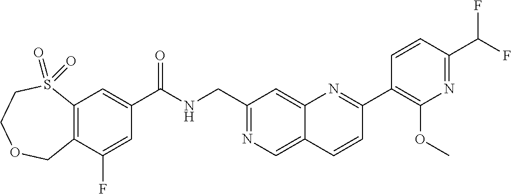 [SMILES: COc1nc(C(F)F)ccc1-c1ccc2cnc(CNC(=O)c3cc(F)c4c(c3)S(=O)(=O)CCOC4)cc2n1] |
| 405 | 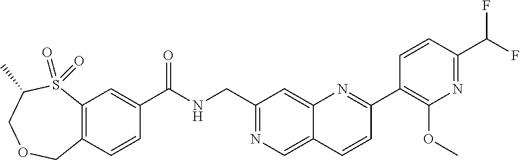 [SMILES: COc1nc(C(F)F)ccc1-c1ccc2cnc(CNC(=O)c3ccc4c(c3)S(=O)(=O)[C@@H](C)COC4)cc2n1] |
| 406 | 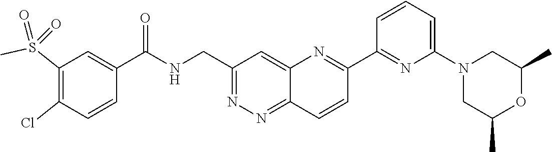 |
| 407 | 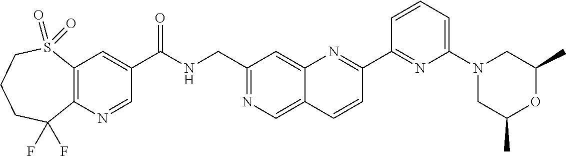 [SMILES: C[C@@H]1CN(c2cccc(-c3ccc4cnc(CNC(=O)c5cnc6c(c5)S(=O)(=O)CCCC6(F)F)cc4n3)n2)C[C@H](C)O1] |
| 408 | 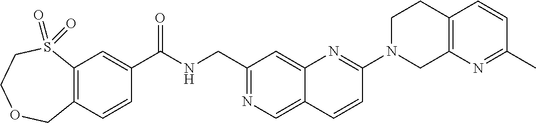 [SMILES: Cc1ccc2c(n1)CN(c1ccc3cnc(CNC(=O)c4ccc5c(c4)S(=O)(=O)CCOC5)cc3n1)CC2] |
| 409 | 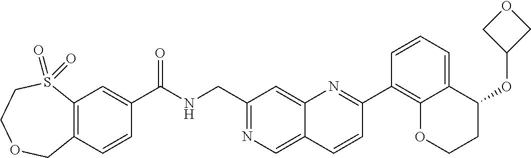 [SMILES: O=C(NCc1cc2nc(-c3cccc4c3OCC[C@H]4OC3COC3)ccc2cn1)c1ccc2c(c1)S(=O)(=O)CCOC2] |
| 410 | 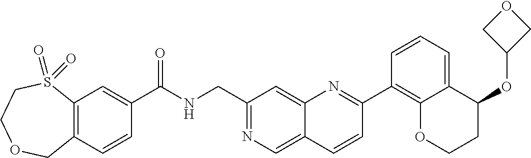 [SMILES: O=C(NCc1cc2nc(-c3cccc4c3OCC[C@@H]4OC3COC3)ccc2cn1)c1ccc2c(c1)S(=O)(=O)CCOC2] |
| 411 | 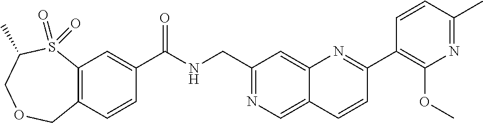 |
| 412 | 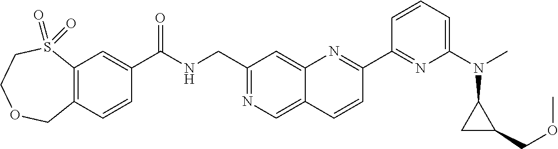 [SMILES: COC[C@H]1C[C@H]1N(C)c1cccc(-c2ccc3cnc(CNC(=O)c4ccc5c(c4)S(=O)(=O)CCOC5)cc3n2)n1] |
| 413 | 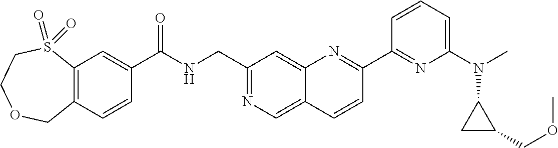 [SMILES: COC[C@@H]1C[C@@H]1N(C)c1cccc(-c2ccc3cnc(CNC(=O)c4ccc5c(c4)S(=O)(=O)CCOC5)cc3n2)n1] |
| 414 | 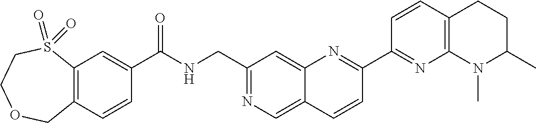 [SMILES: CC1CCc2ccc(-c3ccc4cnc(CNC(=O)c5ccc6c(c5)S(=O)(=O)CCOC6)cc4n3)nc2N1C] |
| 415 | 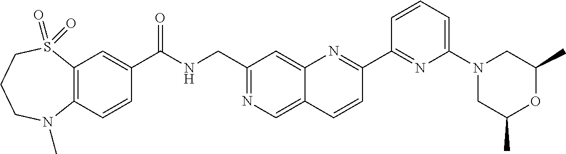 [SMILES: C[C@@H]1CN(c2cccc(-c3ccc4cnc(CNC(=O)c5ccc6c(c5)S(=O)(=O)CCCN6C)cc4n3)n2)C[C@H](C)O1] |
| 416 | 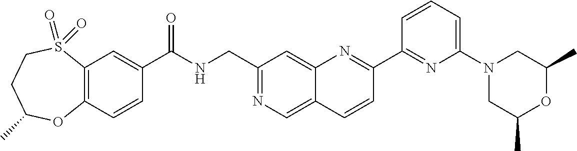 |
| 417 | 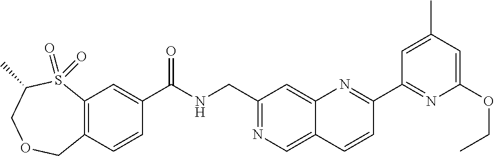 [SMILES: CCOc1cc(C)cc(-c2ccc3cnc(CNC(=O)c4ccc5c(c4)S(=O)(=O)[C@@H](C)COC5)cc3n2)n1] |
| 418 | 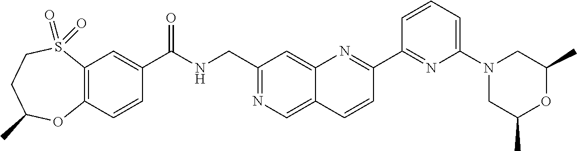 |
| 419 | 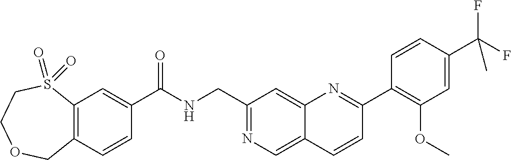 [SMILES: COc1cc(C(C)(F)F)ccc1-c1ccc2cnc(CNC(=O)c3ccc4c(c3)S(=O)(=O)CCOC4)cc2n1] |
| 420 | 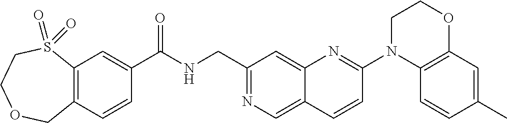 [SMILES: Cc1ccc2c(c1)OCCN2c1ccc2cnc(CNC(=O)c3ccc4c(c3)S(=O)(=O)CCOC4)cc2n1] |
| 421 | 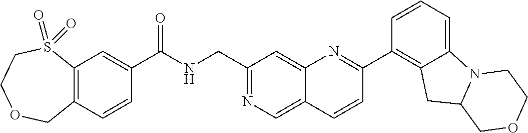 [SMILES: O=C(NCc1cc2nc(-c3cccc4c3CC3COCCN43)ccc2cn1)c1ccc2c(c1)S(=O)(=O)CCOC2] |
| 422 | 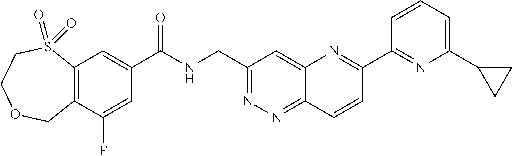 [SMILES: O=C(NCc1cc2nc(-c3cccc(C4CC4)n3)ccc2nn1)c1cc(F)c2c(c1)S(=O)(=O)CCOC2] |
| 423 | 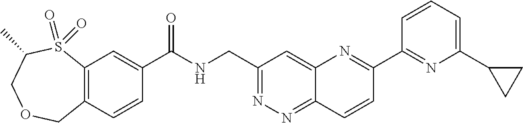 [SMILES: C[C@H]1COCc2ccc(C(=O)NCc3cc4nc(-c5cccc(C6CC6)n5)ccc4nn3)cc2S1(=O)=O] |
| 424 | 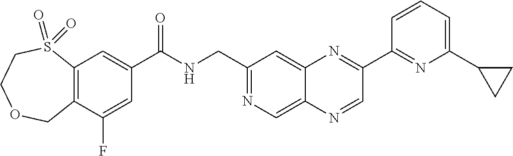 [SMILES: O=C(NCc1cc2nc(-c3cccc(C4CC4)n3)cnc2cn1)c1cc(F)c2c(c1)S(=O)(=O)CCOC2] |
| 425 | 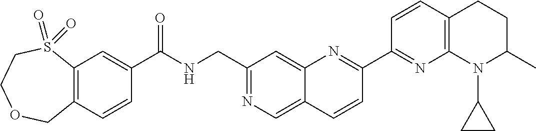 [SMILES: CC1CCc2ccc(-c3ccc4cnc(CNC(=O)c5ccc6c(c5)S(=O)(=O)CCOC6)cc4n3)nc2N1C1CC1] |
| 426 | 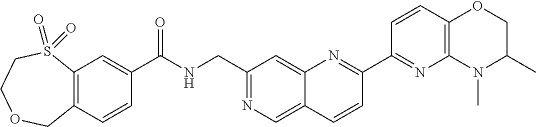 [SMILES: CC1COc2ccc(-c3ccc4cnc(CNC(=O)c5ccc6c(c5)S(=O)(=O)CCOC6)cc4n3)nc2N1C] |
| 427 | 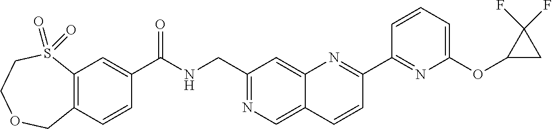 [SMILES: O=C(NCc1cc2nc(-c3cccc(OC4CC4(F)F)n3)ccc2cn1)c1ccc2c(c1)S(=O)(=O)CCOC2] |
| 428 | 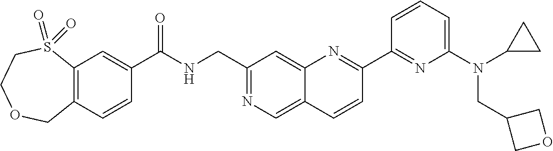 [SMILES: O=C(NCc1cc2nc(-c3cccc(N(CC4COC4)C4CC4)n3)ccc2cn1)c1ccc2c(c1)S(=O)(=O)CCOC2] |
| 429 | 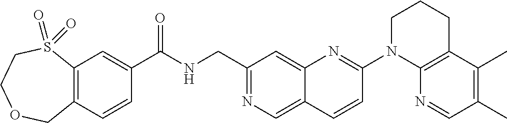 [SMILES: Cc1cnc2c(c1C)CCCN2c1ccc2cnc(CNC(=O)c3ccc4c(c3)S(=O)(=O)CCOC4)cc2n1] |
| 430 | 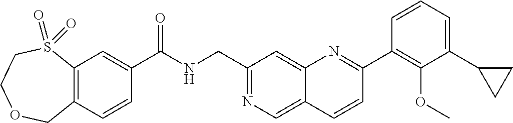 [SMILES: COc1c(-c2ccc3cnc(CNC(=O)c4ccc5c(c4)S(=O)(=O)CCOC5)cc3n2)cccc1C1CC1] |
| 431 | 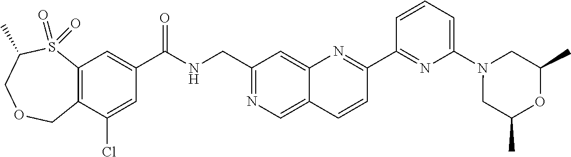 |
| 432 | 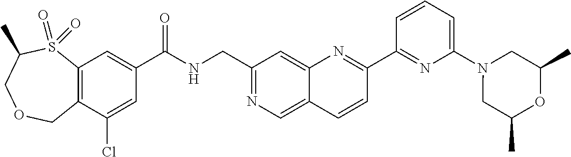 |
| 433 | 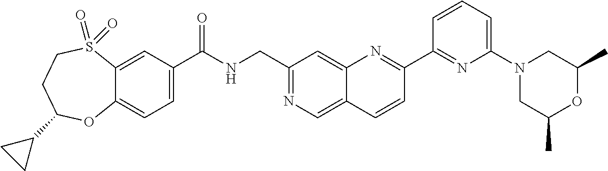 [SMILES: C[C@@H]1CN(c2cccc(-c3ccc4cnc(CNC(=O)c5ccc6c(c5)S(=O)(=O)CC[C@@H](C5CC5)O6)cc4n3)n2)C[C@H](C)O1] |
| 434 | 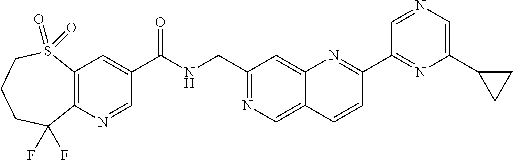 [SMILES: O=C(NCc1cc2nc(-c3cncc(C4CC4)n3)ccc2cn1)c1cnc2c(c1)S(=O)(=O)CCCC2(F)F] |
| 435 | 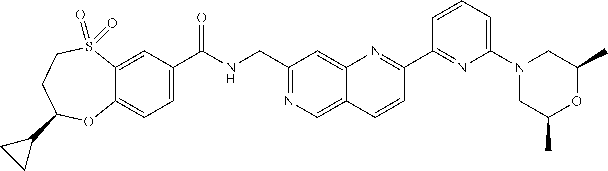 [SMILES: C[C@@H]1CN(c2cccc(-c3ccc4cnc(CNC(=O)c5ccc6c(c5)S(=O)(=O)CC[C@H](C5CC5)O6)cc4n3)n2)C[C@H](C)O1] |
| 436 | 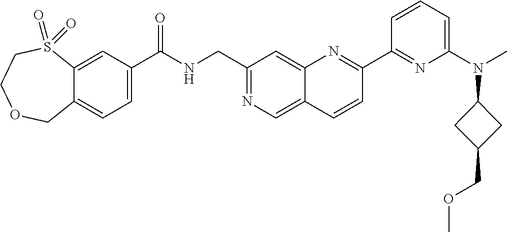 [SMILES: COC[C@H]1C[C@@H](N(C)c2cccc(-c3ccc4cnc(CNC(=O)c5ccc6c(c5)S(=O)(=O)CCOC6)cc4n3)n2)C1] |
| 437 | 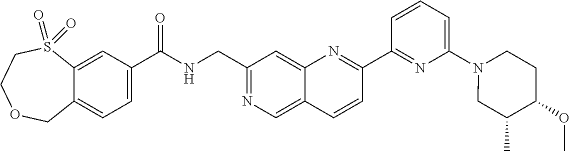 [SMILES: CO[C@H]1CCN(c2cccc(-c3ccc4cnc(CNC(=O)c5ccc6c(c5)S(=O)(=O)CCOC6)cc4n3)n2)C[C@H]1C] |
| 438 | 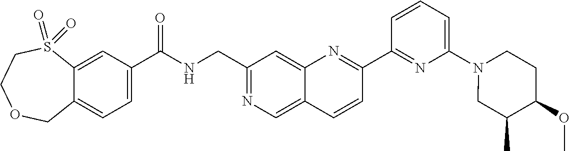 [SMILES: CO[C@@H]1CCN(c2cccc(-c3ccc4cnc(CNC(=O)c5ccc6c(c5)S(=O)(=O)CCOC6)cc4n3)n2)C[C@@H]1C] |
| 439 | 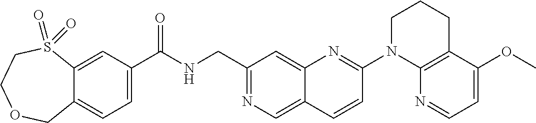 [SMILES: COc1ccnc2c1CCCN2c1ccc2cnc(CNC(=O)c3ccc4c(c3)S(=O)(=O)CCOC4)cc2n1] |
| 440 | 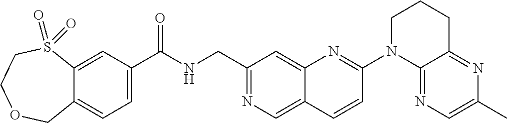 [SMILES: Cc1cnc2c(n1)CCCN2c1ccc2cnc(CNC(=O)c3ccc4c(c3)S(=O)(=O)CCOC4)cc2n1] |
| 441 | 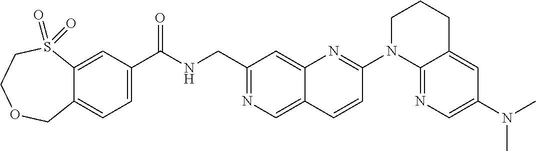 [SMILES: CN(C)c1cnc2c(c1)CCCN2c1ccc2cnc(CNC(=O)c3ccc4c(c3)S(=O)(=O)CCOC4)cc2n1] |
| 442 | 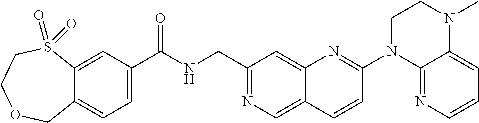 [SMILES: CN1CCN(c2ccc3cnc(CNC(=O)c4ccc5c(c4)S(=O)(=O)CCOC5)cc3n2)c2ncccc21] |
| 443 | 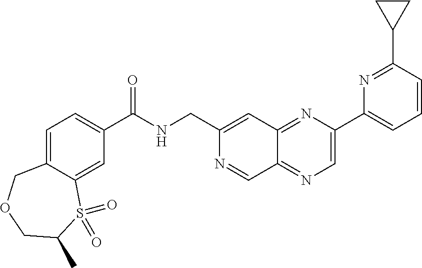 [SMILES: C[C@H]1COCc2ccc(C(=O)NCc3cc4nc(-c5cccc(C6CC6)n5)cnc4cn3)cc2S1(=O)=O] |
| 444 | 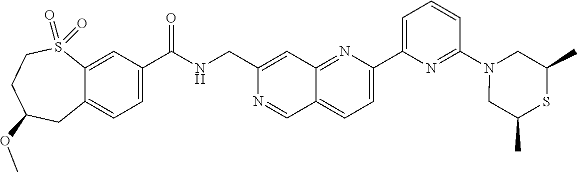 [SMILES: CO[C@H]1CCS(=O)(=O)c2cc(C(=O)NCc3cc4nc(-c5cccc(N6C[C@@H](C)S[C@@H](C)C6)n5)ccc4cn3)ccc2C1] |
| 445 | 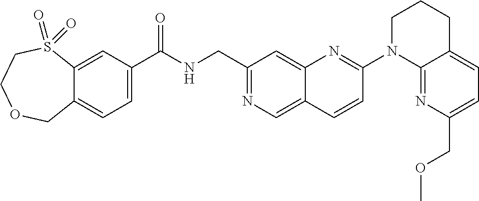 [SMILES: COCc1ccc2c(n1)N(c1ccc3cnc(CNC(=O)c4ccc5c(c4)S(=O)(=O)CCOC5)cc3n1)CCC2] |
| 446 | 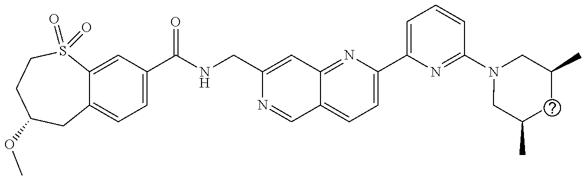 [SMILES: CO[C@@H]1CCS(=O)(=O)c2cc(C(=O)NCc3cc4nc(-c5cccc(N6C[C@H](C)C[C@H](C)C6)n5)ccc4cn3)ccc2C1] |
| 447 | 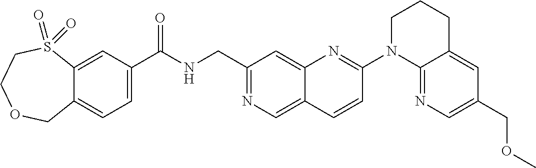 [SMILES: COCc1cnc2c(c1)CCCN2c1ccc2cnc(CNC(=O)c3ccc4c(c3)S(=O)(=O)CCOC4)cc2n1] |
| 448 | 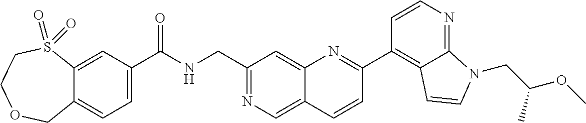 [SMILES: CO[C@H](C)Cn1ccc2c(-c3ccc4cnc(CNC(=O)c5ccc6c(c5)S(=O)(=O)CCOC6)cc4n3)ccnc21] |
| 449 | 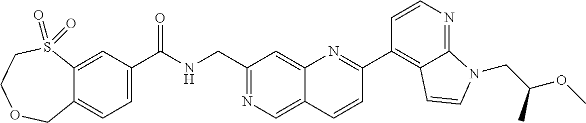 [SMILES: CO[C@@H](C)Cn1ccc2c(-c3ccc4cnc(CNC(=O)c5ccc6c(c5)S(=O)(=O)CCOC6)cc4n3)ccnc21] |
| 450 | 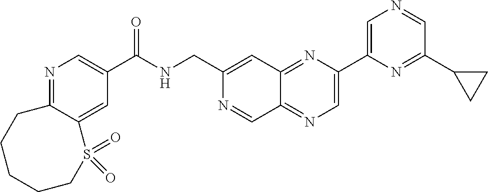 [SMILES: O=C(NCc1cc2nc(-c3cncc(C4CC4)n3)cnc2cn1)c1cnc2c(c1)S(=O)(=O)CCCCC2] |
| 451 | 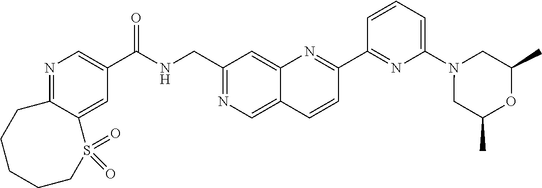 [SMILES: C[C@@H]1CN(c2cccc(-c3ccc4cnc(CNC(=O)c5cnc6c(c5)S(=O)(=O)CCCCC6)cc4n3)n2)C[C@H](C)O1] |
| 452 | 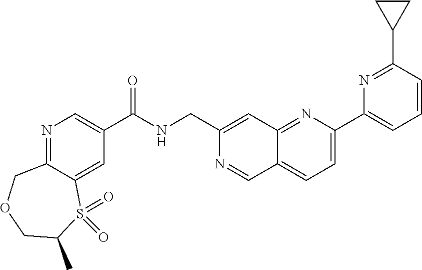 [SMILES: C[C@H]1COCc2ncc(C(=O)NCc3cc4nc(-c5cccc(C6CC6)n5)ccc4cn3)cc2S1(=O)=O] |
| 453 | 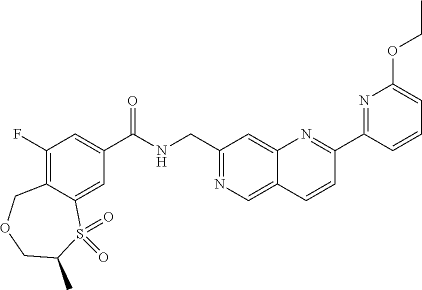 [SMILES: CCOc1cccc(-c2ccc3cnc(CNC(=O)c4cc(F)c5c(c4)S(=O)(=O)[C@@H](C)COC5)cc3n2)n1] |
| 454 | 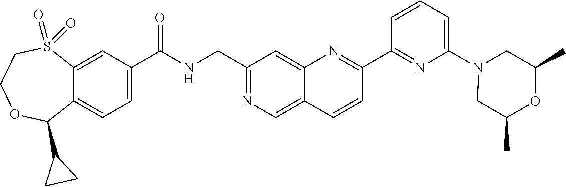 [SMILES: C[C@@H]1CN(c2cccc(-c3ccc4cnc(CNC(=O)c5ccc6c(c5)S(=O)(=O)CCO[C@@H]6C5CC5)cc4n3)n2)C[C@H](C)O1] |
| 455 | 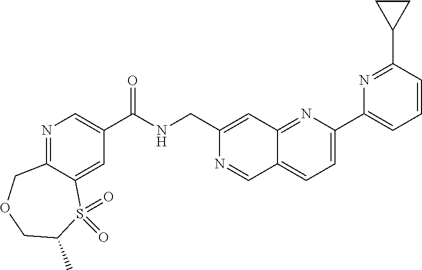 [SMILES: C[C@@H]1COCc2ncc(C(=O)NCc3cc4nc(-c5cccc(C6CC6)n5)ccc4cn3)cc2S1(=O)=O] |
| 456 | 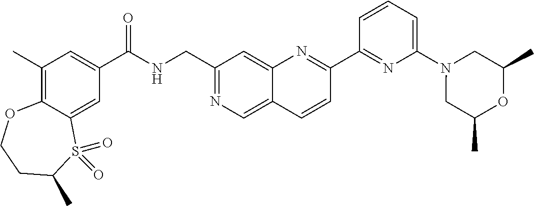 [SMILES: Cc1cc(C(=O)NCc2cc3nc(-c4cccc(N5C[C@@H](C)O[C@@H](C)C5)n4)ccc3cn2)cc2c1OCC[C@H](C)S2(=O)=O] |
| 457 | 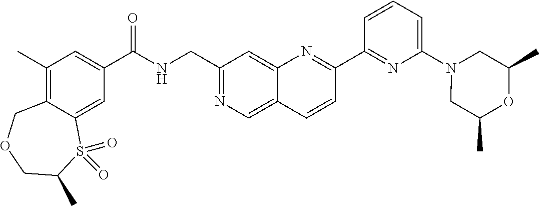 [SMILES: Cc1cc(C(=O)NCc2cc3nc(-c4cccc(N5C[C@@H](C)O[C@@H](C)C5)n4)ccc3cn2)cc2c1COC[C@H](C)S2(=O)=O] |
| 458 | 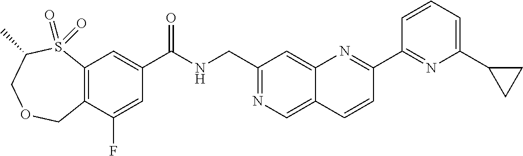 [SMILES: C[C@H]1COCc2c(F)cc(C(=O)NCc3cc4nc(-c5cccc(C6CC6)n5)ccc4cn3)cc2S1(=O)=O] |
| 459 | 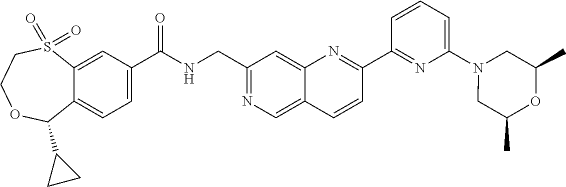 [SMILES: C[C@@H]1CN(c2cccc(-c3ccc4cnc(CNC(=O)c5ccc6c(c5)S(=O)(=O)CCO[C@H]6C5CC5)cc4n3)n2)C[C@H](C)O1] |
| 460 | 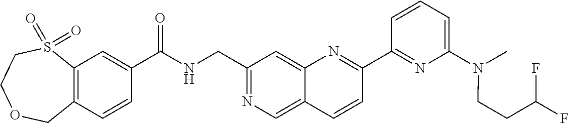 [SMILES: CN(CCC(F)F)c1cccc(-c2ccc3cnc(CNC(=O)c4ccc5c(c4)S(=O)(=O)CCOC5)cc3n2)n1] |
| 461 | 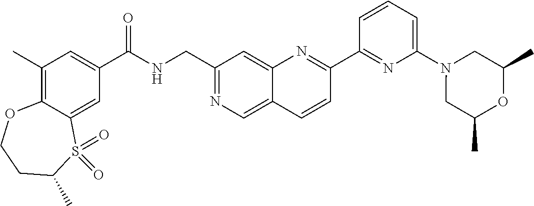 [SMILES: Cc1cc(C(=O)NCc2cc3nc(-c4cccc(N5C[C@@H](C)O[C@@H](C)C5)n4)ccc3cn2)cc2c1OCC[C@@H](C)S2(=O)=O] |
| 462 | 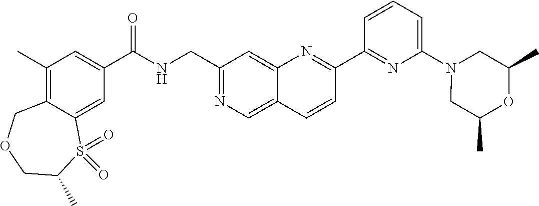 [SMILES: Cc1cc(C(=O)NCc2cc3nc(-c4cccc(N5C[C@@H](C)O[C@@H](C)C5)n4)ccc3cn2)cc2c1COC[C@@H](C)S2(=O)=O] |
| 463 | 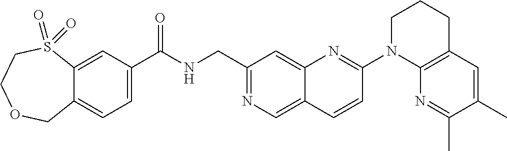 [SMILES: Cc1cc2c(nc1C)N(c1ccc3cnc(CNC(=O)c4ccc5c(c4)S(=O)(=O)CCOC5)cc3n1)CCC2] |
| 464 | 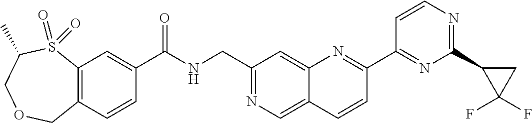 [SMILES: C[C@H]1COCc2ccc(C(=O)NCc3cc4nc(-c5ccnc([C@H]6CC6(F)F)n5)ccc4cn3)cc2S1(=O)=O] |
| 465 | 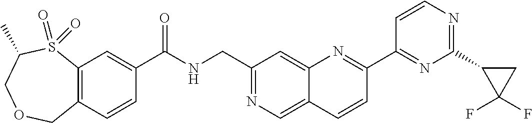 [SMILES: C[C@H]1COCc2ccc(C(=O)NCc3cc4nc(-c5ccnc([C@@H]6CC6(F)F)n5)ccc4cn3)cc2S1(=O)=O] |
| 466 | 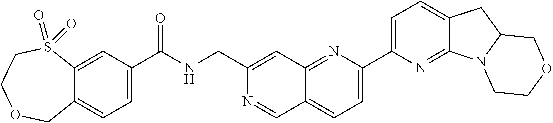 [SMILES: O=C(NCc1cc2nc(-c3ccc4c(n3)N3CCOCC3C4)ccc2cn1)c1ccc2c(c1)S(=O)(=O)CCOC2] |
| 467 | 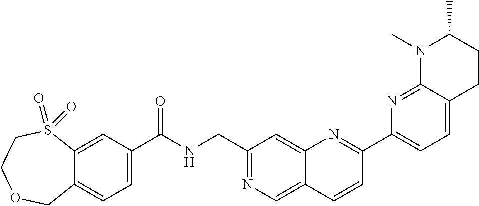 [SMILES: C[C@@H]1CCc2ccc(-c3ccc4cnc(CNC(=O)c5ccc6c(c5)S(=O)(=O)CCOC6)cc4n3)nc2N1C] |
| 468 | 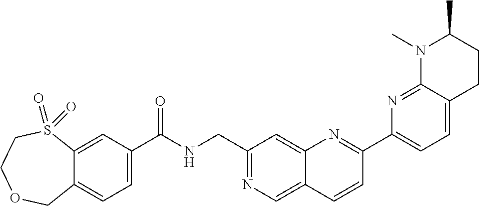 [SMILES: C[C@H]1CCc2ccc(-c3ccc4cnc(CNC(=O)c5ccc6c(c5)S(=O)(=O)CCOC6)cc4n3)nc2N1C] |
| 469 | 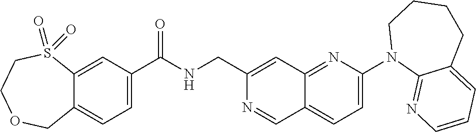 [SMILES: O=C(NCc1cc2nc(N3CCCCc4cccnc43)ccc2cn1)c1ccc2c(c1)S(=O)(=O)CCOC2] |
| 470 | 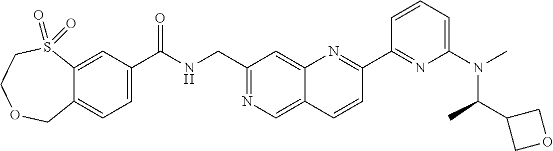 [SMILES: C[C@H](C1COC1)N(C)c1cccc(-c2ccc3cnc(CNC(=O)c4ccc5c(c4)S(=O)(=O)CCOC5)cc3n2)n1] |
| 471 | 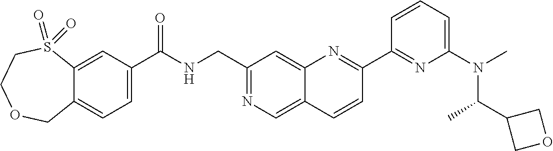 [SMILES: C[C@@H](C1COC1)N(C)c1cccc(-c2ccc3cnc(CNC(=O)c4ccc5c(c4)S(=O)(=O)CCOC5)cc3n2)n1] |
| 472 | 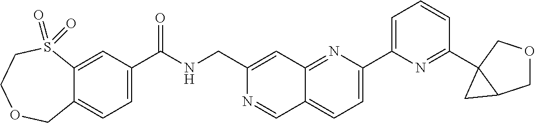 [SMILES: O=C(NCc1cc2nc(-c3cccc(C45COCC4C5)n3)ccc2cn1)c1ccc2c(c1)S(=O)(=O)CCOC2] |
| 473 | 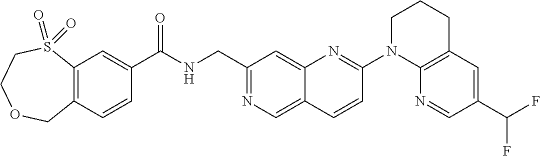 [SMILES: O=C(NCc1cc2nc(N3CCCc4cc(C(F)F)cnc43)ccc2cn1)c1ccc2c(c1)S(=O)(=O)CCOC2] |
| 474 | 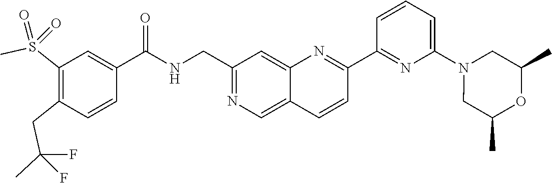 |
| 475 | 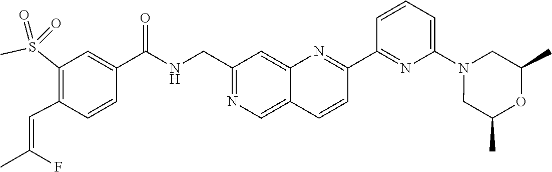 |
| 476 | 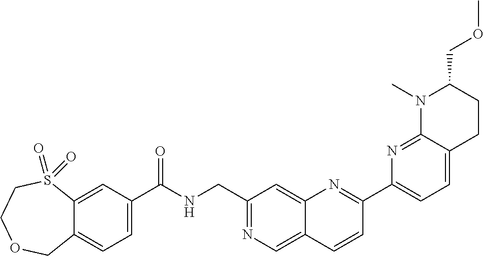 [SMILES: COC[C@@H]1CCc2ccc(-c3ccc4cnc(CNC(=O)c5ccc6c(c5)S(=O)(=O)CCOC6)cc4n3)nc2N1C] |
| 477 | 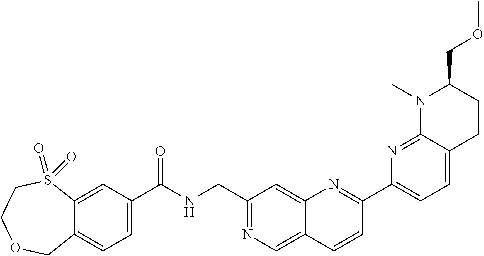 [SMILES: COC[C@H]1CCc2ccc(-c3ccc4cnc(CNC(=O)c5ccc6c(c5)S(=O)(=O)CCOC6)cc4n3)nc2N1C] |
| 478 | 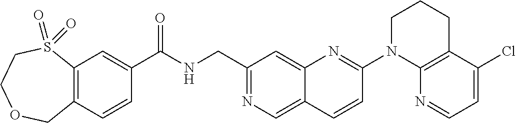 [SMILES: O=C(NCc1cc2nc(N3CCCc4c(Cl)ccnc43)ccc2cn1)c1ccc2c(c1)S(=O)(=O)CCOC2] |
| 479 | 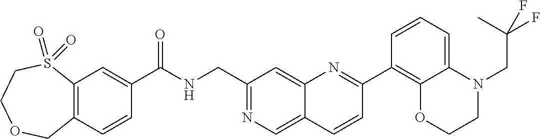 [SMILES: CC(F)(F)CN1CCOc2c(-c3ccc4cnc(CNC(=O)c5ccc6c(c5)S(=O)(=O)CCOC6)cc4n3)cccc21] |
| 480 | 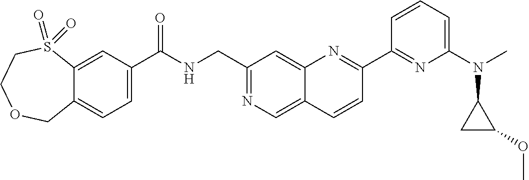 [SMILES: CO[C@@H]1C[C@H]1N(C)c1cccc(-c2ccc3cnc(CNC(=O)c4ccc5c(c4)S(=O)(=O)CCOC5)cc3n2)n1] |
| 481 | 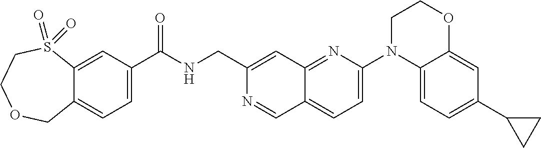 [SMILES: O=C(NCc1cc2nc(N3CCOc4cc(C5CC5)ccc43)ccc2cn1)c1ccc2c(c1)S(=O)(=O)CCOC2] |
| 482 | 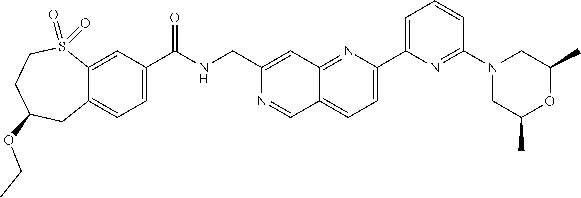 [SMILES: CCO[C@H]1CCS(=O)(=O)c2cc(C(=O)NCc3cc4nc(-c5cccc(N6C[C@@H](C)O[C@@H](C)C6)n5)ccc4cn3)ccc2C1] |
| 483 | 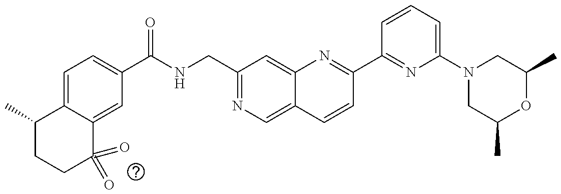 |
| 484 | 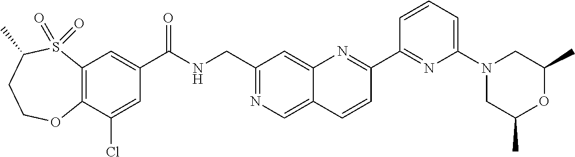 |
| 485 | 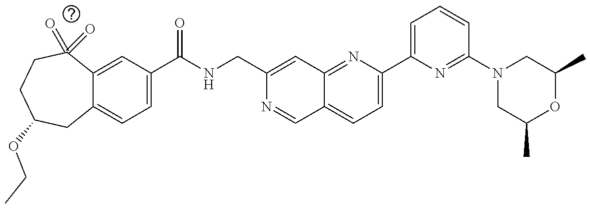 |
| 486 | 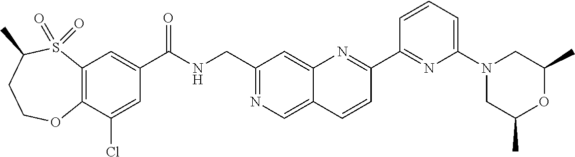 |
| 487 | 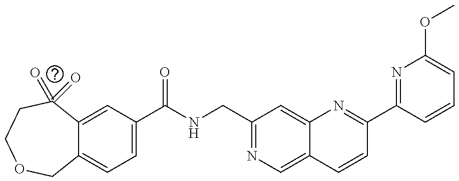 |
| 488 | 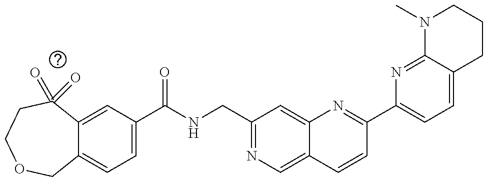 |
| 489 | 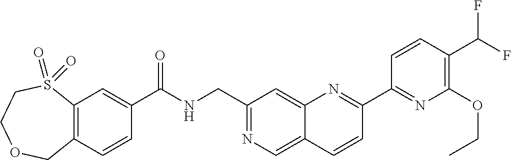 [SMILES: CCOc1nc(-c2ccc3cnc(CNC(=O)c4ccc5c(c4)S(=O)(=O)CCOC5)cc3n2)ccc1C(F)F] |
| 490 | 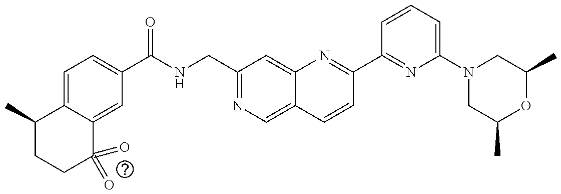 |
| 491 | 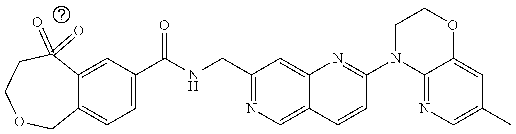 |
| 492 | 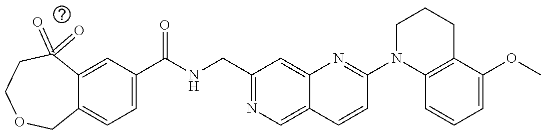 |
| 493 | 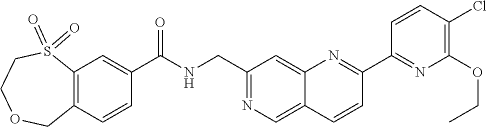 [SMILES: CCOc1nc(-c2ccc3cnc(CNC(=O)c4ccc5c(c4)S(=O)(=O)CCOC5)cc3n2)ccc1Cl] |
| 494 | 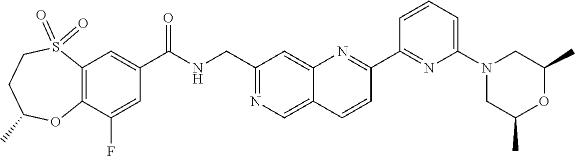 |
| 495 | 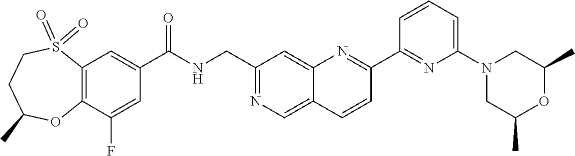 |
| 496 | 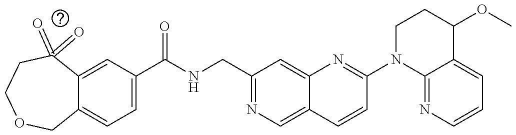 |
| 497 | 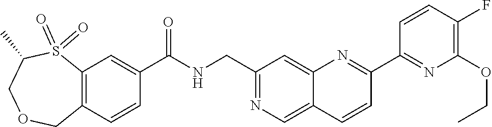 [SMILES: CCOc1nc(-c2ccc3cnc(CNC(=O)c4ccc5c(c4)S(=O)(=O)[C@@H](C)COC5)cc3n2)ccc1F] |
| 498 | 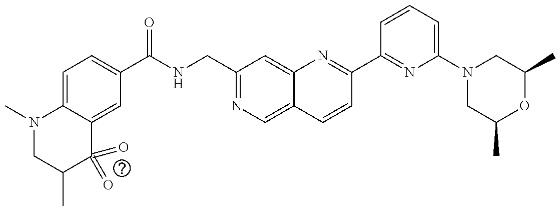 |
| 499 | 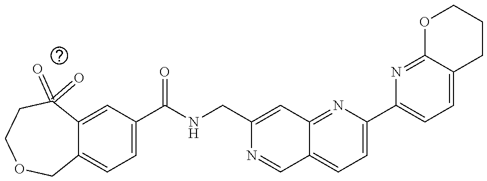 |
| 500 | 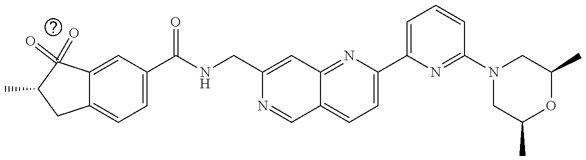 |
| 501 | 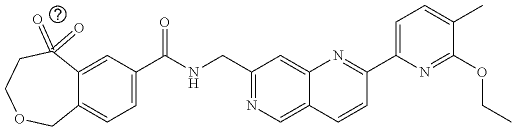 |
| 502 | 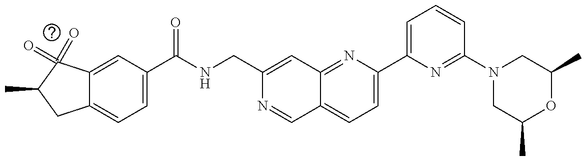 |
| 503 | 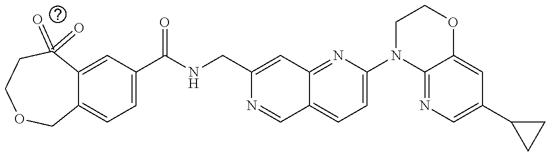 |
| 504 | 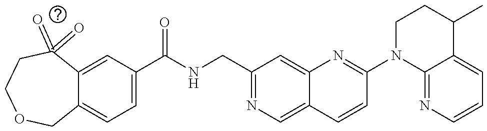 |
| 505 | 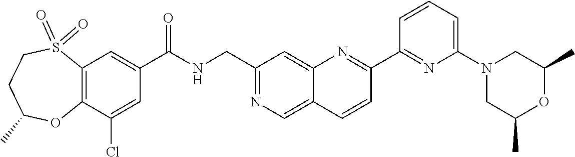 |
| 506 | 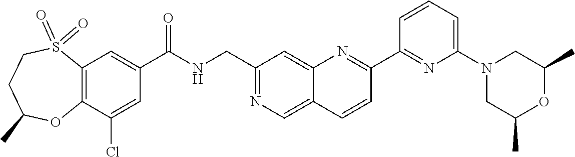 |
| 507 | 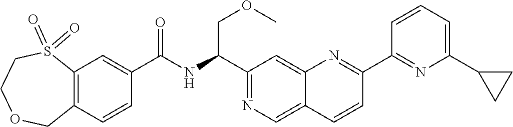 [SMILES: COC[C@H](NC(=O)c1ccc2c(c1)S(=O)(=O)CCOC2)c1cc2nc(-c3cccc(C4CC4)n3)ccc2cn1] |
| 508 | 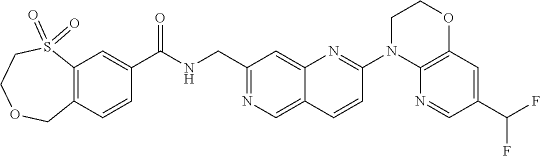 [SMILES: O=C(NCc1cc2nc(N3CCOc4cc(C(F)F)cnc43)ccc2cn1)c1ccc2c(c1)S(=O)(=O)CCOC2] |
| 509 | 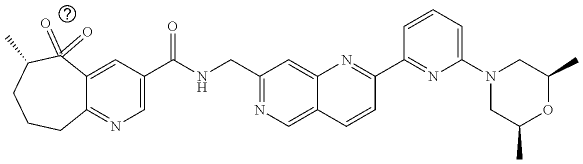 |
| 510 | 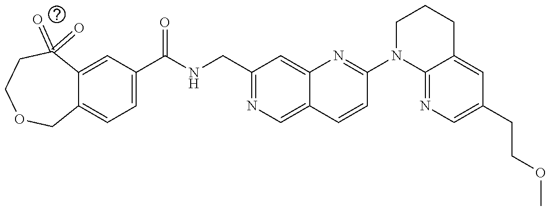 |
| 511 | 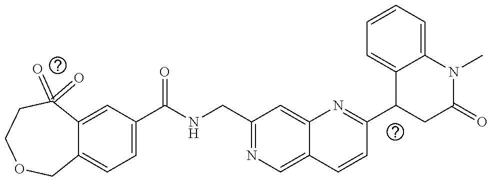 |
| 512 | 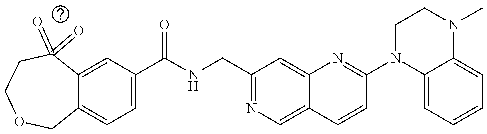 |
| 513 | 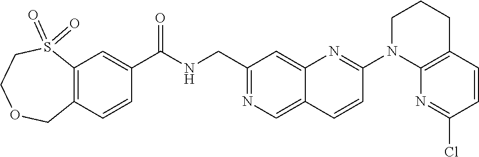 [SMILES: O=C(NCc1cc2nc(N3CCCc4ccc(Cl)nc43)ccc2cn1)c1ccc2c(c1)S(=O)(=O)CCOC2] |
| 514 | 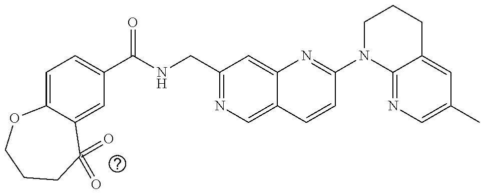 |
| 515 | 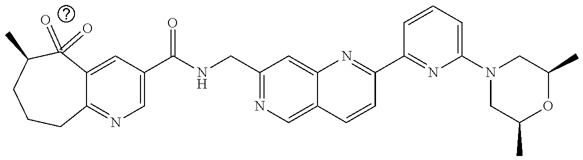 |
| 516 | 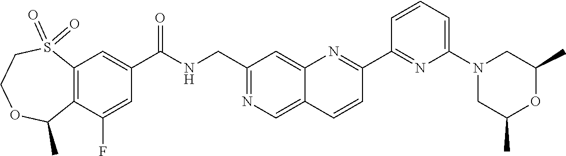 [SMILES: C[C@@H]1CN(c2cccc(-c3ccc4cnc(CNC(=O)c5cc(F)c6c(c5)S(=O)(=O)CCO[C@@H]6C)cc4n3)n2)C[C@H](C)O1] |
| 517 | 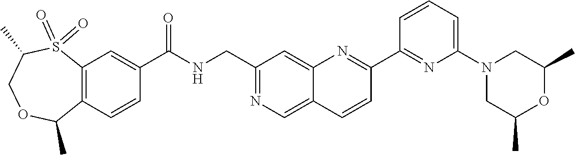 |
| 518 | 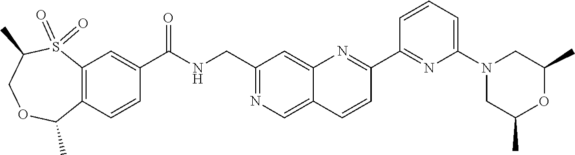 |
| 519 | 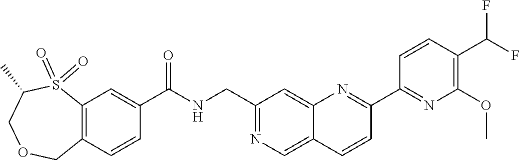 [SMILES: COc1nc(-c2ccc3cnc(CNC(=O)c4ccc5c(c4)S(=O)(=O)[C@@H](C)COC5)cc3n2)ccc1C(F)F] |
| 520 | 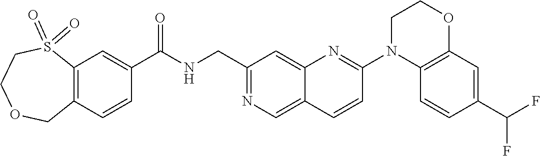 [SMILES: O=C(NCc1cc2nc(N3CCOc4cc(C(F)F)ccc43)ccc2cn1)c1ccc2c(c1)S(=O)(=O)CCOC2] |
| 521 | 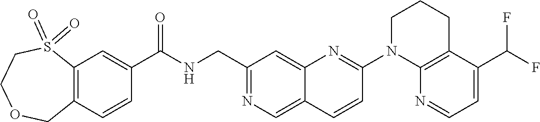 [SMILES: O=C(NCc1cc2nc(N3CCCc4c(C(F)F)ccnc43)ccc2cn1)c1ccc2c(c1)S(=O)(=O)CCOC2] |
| 522 | 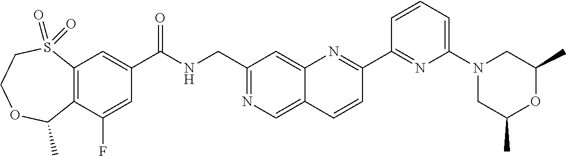 [SMILES: C[C@@H]1CN(c2cccc(-c3ccc4cnc(CNC(=O)c5cc(F)c6c(c5)S(=O)(=O)CCO[C@H]6C)cc4n3)n2)C[C@H](C)O1] |
| 523 | 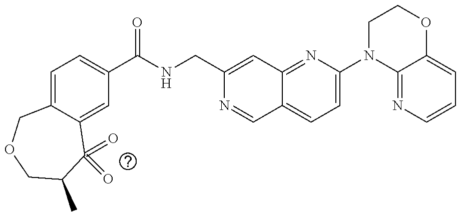 |
| 524 | 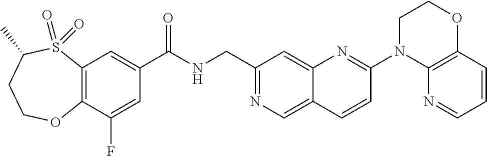 [SMILES: C[C@H]1CCOc2c(F)cc(C(=O)NCc3cc4nc(N5CCOc6cccnc65)ccc4cn3)cc2S1(=O)=O] |
| 525 | 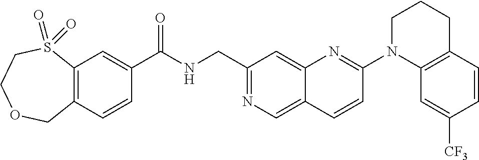 [SMILES: O=C(NCc1cc2nc(N3CCCc4ccc(C(F)(F)F)cc43)ccc2cn1)c1ccc2c(c1)S(=O)(=O)CCOC2] |
| 526 | 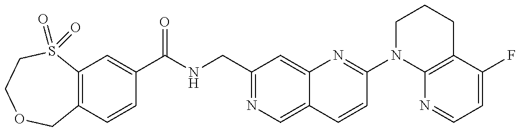 [SMILES: O=C(NCc1cc2nc(N3CCCc4c(F)ccnc43)ccc2cn1)c1ccc2c(c1)S(=O)(=O)CCOC2] |
| 527 | 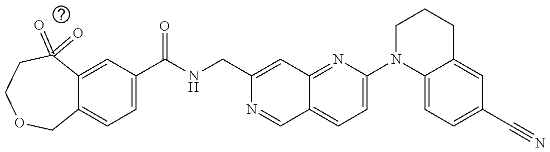 |
| 528 | 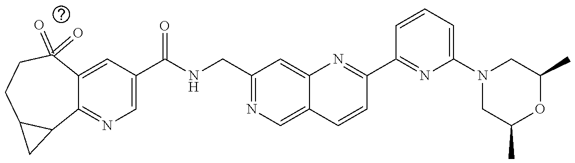 |
| 529 | 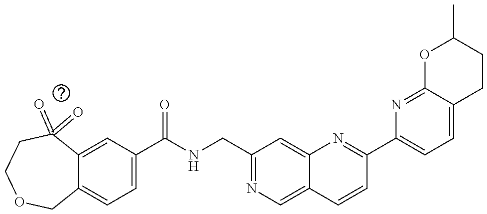 |
| 530 | 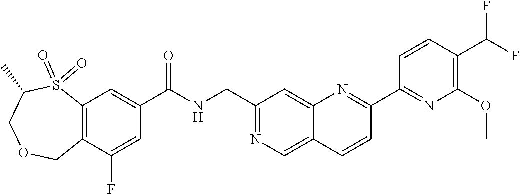 [SMILES: COc1nc(-c2ccc3cnc(CNC(=O)c4cc(F)c5c(c4)S(=O)(=O)[C@@H](C)COC5)cc3n2)ccc1C(F)F] |
| 531 | 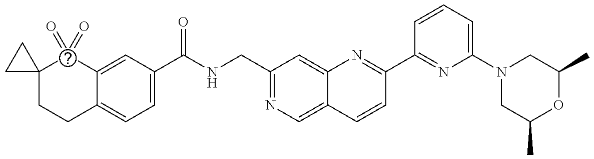 |
| 532 | 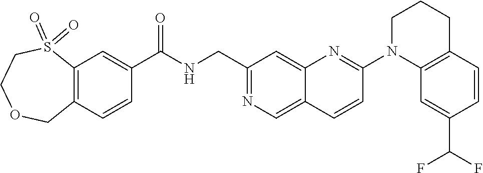 [SMILES: O=C(NCc1cc2nc(N3CCCc4ccc(C(F)F)cc43)ccc2cn1)c1ccc2c(c1)S(=O)(=O)CCOC2] |
| 533 | 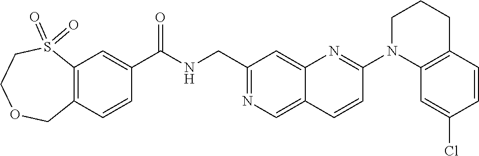 [SMILES: O=C(NCc1cc2nc(N3CCCc4ccc(Cl)cc43)ccc2cn1)c1ccc2c(c1)S(=O)(=O)CCOC2] |
| 534 | 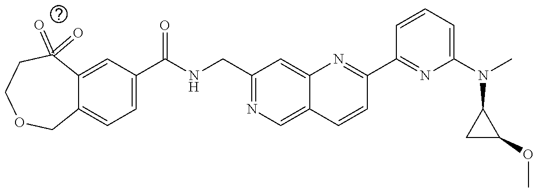 |
| 535 | 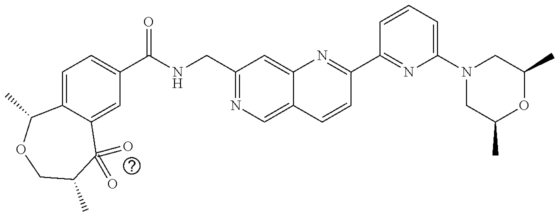 |
| 536 | 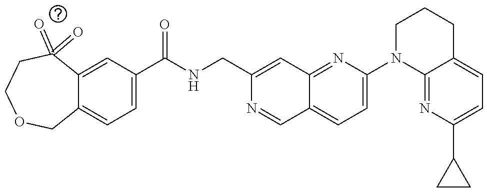 |
| 537 | 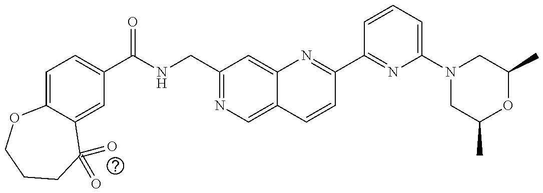 |
| 538 | 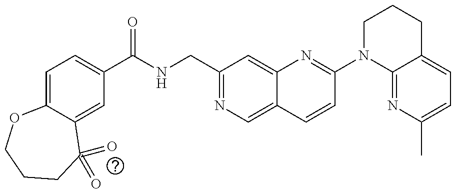 |
| 539 | 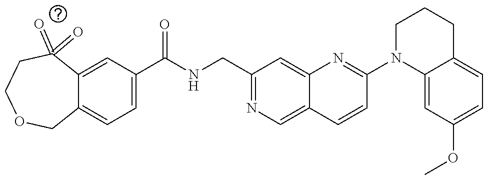 |
| 540 | 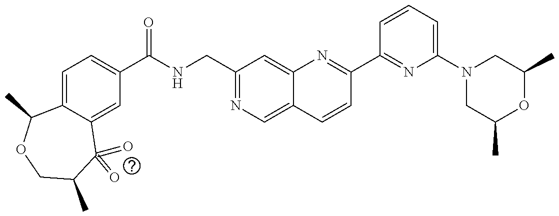 |
| 541 | 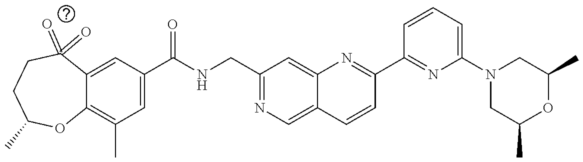 |
| 542 | 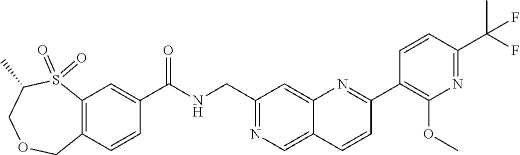 [SMILES: COc1nc(C(C)(F)F)ccc1-c1ccc2cnc(CNC(=O)c3ccc4c(c3)S(=O)(=O)[C@@H](C)COC4)cc2n1] |
| 543 | 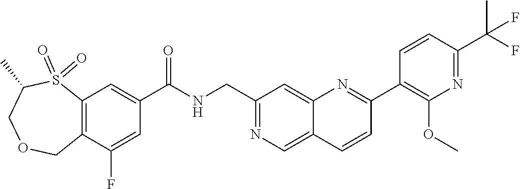 [SMILES: COc1nc(C(C)(F)F)ccc1-c1ccc2cnc(CNC(=O)c3cc(F)c4c(c3)S(=O)(=O)[C@@H](C)COC4)cc2n1] |
| 544 | 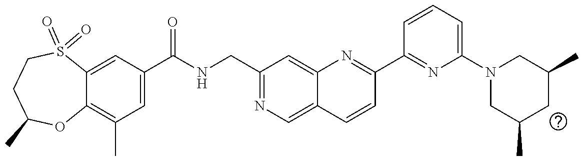 |
| 545 | 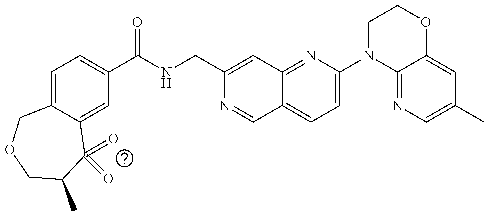 |
| 546 | 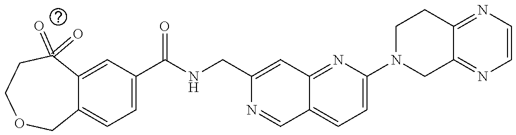 |
| 547 | 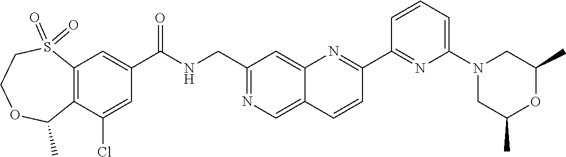 [SMILES: C[C@@H]1CN(c2cccc(-c3ccc4cnc(CNC(=O)c5cc(Cl)c6c(c5)S(=O)(=O)CCO[C@H]6C)cc4n3)n2)C[C@H](C)O1] |
| 548 | 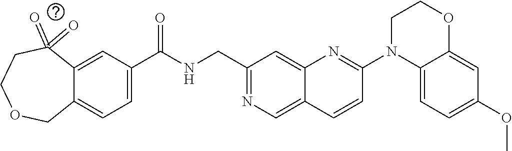 |
| 549 | 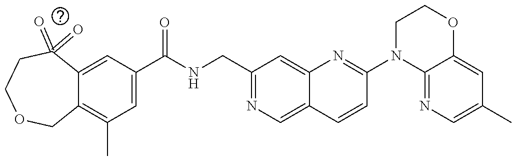 |
| 550 | 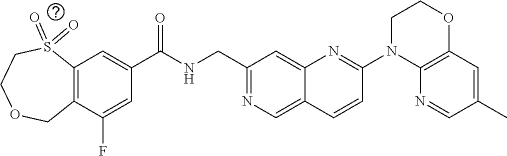 [SMILES: Cc1cnc2c(c1)OCCN2c1ccc2cnc(CNC(=O)c3cc(F)c4c(c3)S(=O)(=O)CCOC4)cc2n1] |
| 551 | 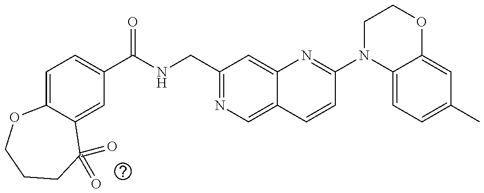 |
| 552 | 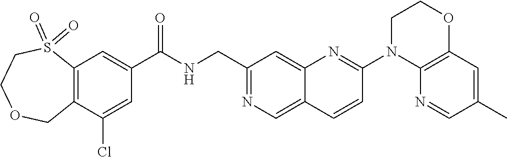 [SMILES: Cc1cnc2c(c1)OCCN2c1ccc2cnc(CNC(=O)c3cc(Cl)c4c(c3)S(=O)(=O)CCOC4)cc2n1] |
| 553 | 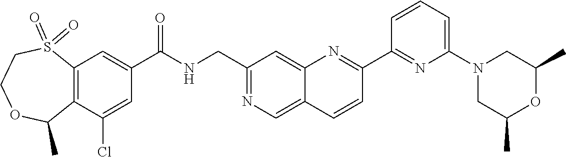 [SMILES: C[C@@H]1CN(c2cccc(-c3ccc4cnc(CNC(=O)c5cc(Cl)c6c(c5)S(=O)(=O)CCO[C@@H]6C)cc4n3)n2)C[C@H](C)O1] |
| 554 | 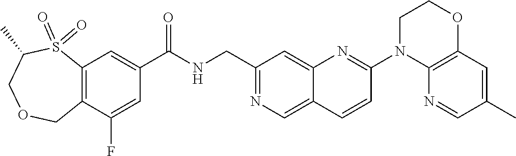 |
| 555 | 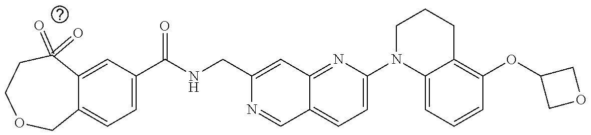 |
| 556 | 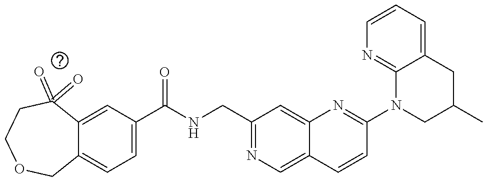 |
| 557 | 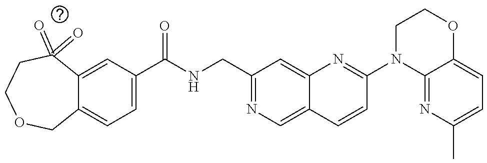 |
| 558 | 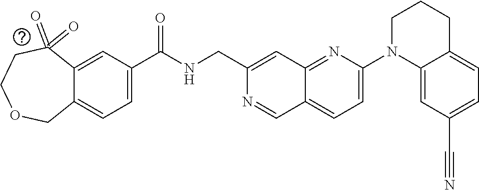 |
| 559 | 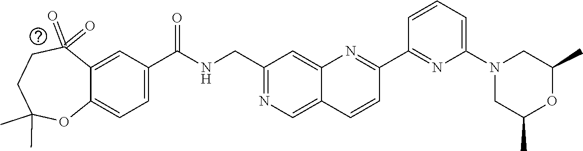 |
| 560 | 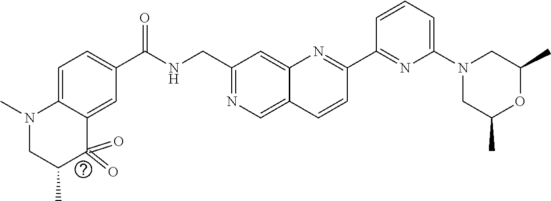 |
| 561 | 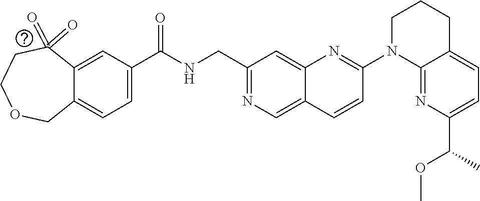 |
| 562 | 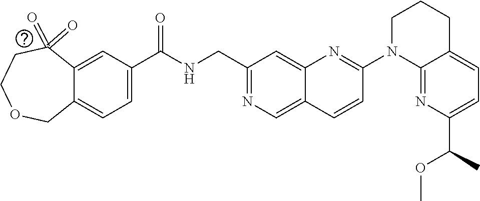 |
| 563 | 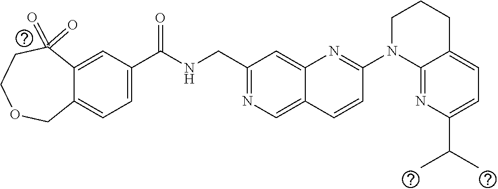 |
| 564 | 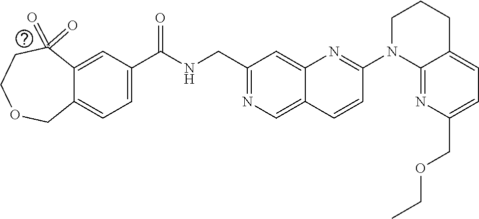 |
| 565 | 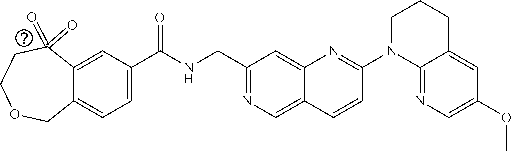 |
| 566 | 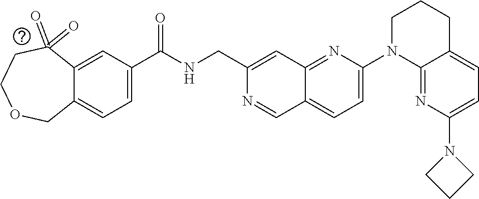 |
| 567 | 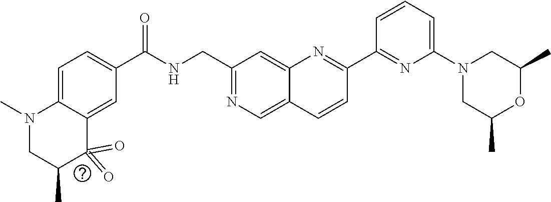 |
| 568 | 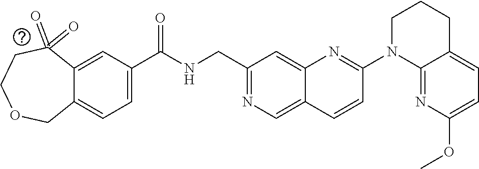 |
| 569 | 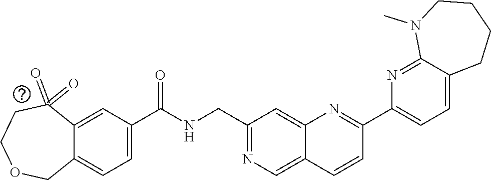 |
| 570 | 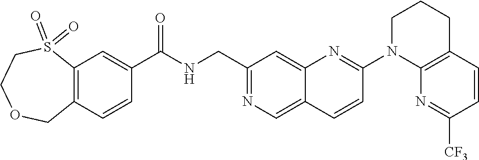 [SMILES: O=C(NCc1cc2nc(N3CCCc4ccc(C(F)(F)F)nc43)ccc2cn1)c1ccc2c(c1)S(=O)(=O)CCOC2] |
| 571 | 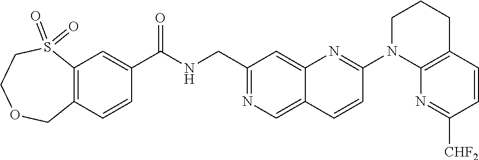 [SMILES: O=C(NCc1cc2nc(N3CCCc4ccc(C(F)F)nc43)ccc2cn1)c1ccc2c(c1)S(=O)(=O)CCOC2] |
| 572 | 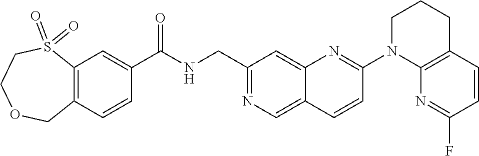 [SMILES: O=C(NCc1cc2nc(N3CCCc4ccc(F)nc43)ccc2cn1)c1ccc2c(c1)S(=O)(=O)CCOC2] |
| 573 | 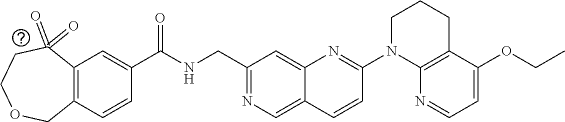 |
| 574 | 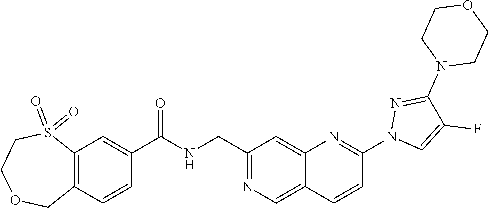 [SMILES: O=C(NCc1cc2nc(-n3cc(F)c(N4CCOCC4)n3)ccc2cn1)c1ccc2c(c1)S(=O)(=O)CCOC2] |
| 575 | 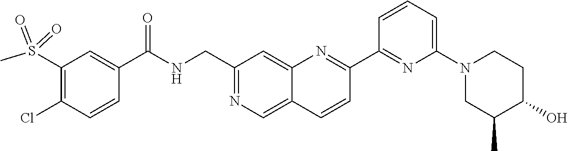 [SMILES: C[C@H]1CN(c2cccc(-c3ccc4cnc(CNC(=O)c5ccc(Cl)c(S(C)(=O)=O)c5)cc4n3)n2)CC[C@@H]1O] |
| 576 | 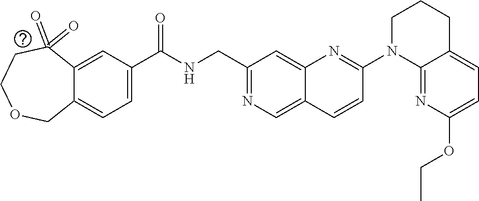 |
| 577 | 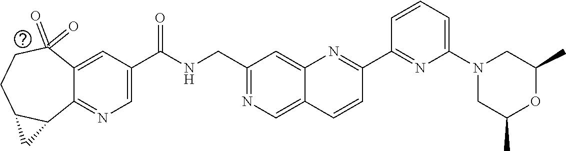 |
| 578 | 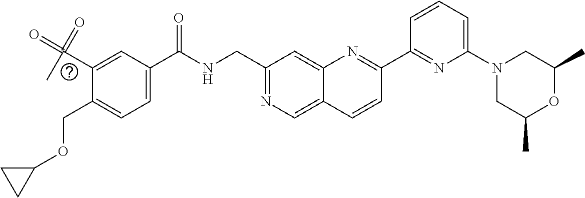 |
| 579 | 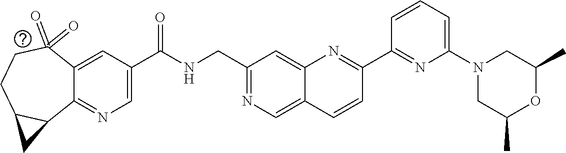 |
| 580 | 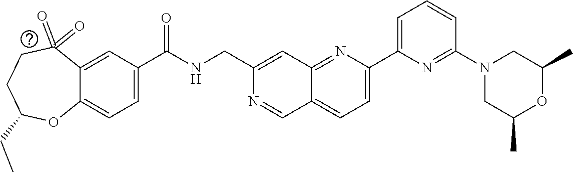 |
| 581 | 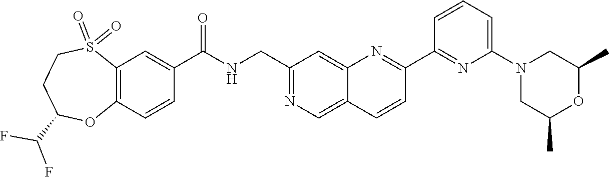 [SMILES: C[C@@H]1CN(c2cccc(-c3ccc4cnc(CNC(=O)c5ccc6c(c5)S(=O)(=O)CC[C@@H](C(F)F)O6)cc4n3)n2)C[C@H](C)O1] |
| 582 | 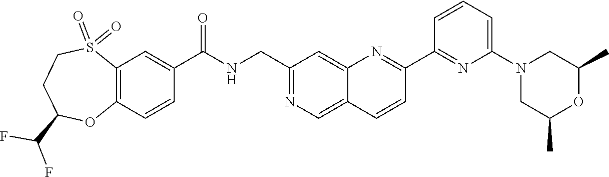 [SMILES: C[C@@H]1CN(c2cccc(-c3ccc4cnc(CNC(=O)c5ccc6c(c5)S(=O)(=O)CC[C@H](C(F)F)O6)cc4n3)n2)C[C@H](C)O1] |
| 583 | 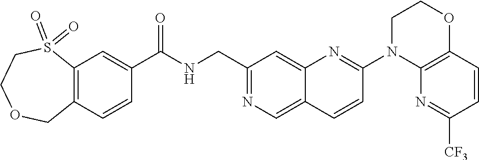 [SMILES: O=C(NCc1cc2nc(N3CCOc4ccc(C(F)(F)F)nc43)ccc2cn1)c1ccc2c(c1)S(=O)(=O)CCOC2] |
| 584 | 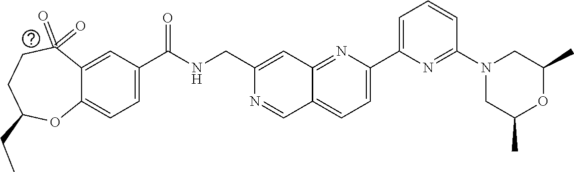 |
| 585 | 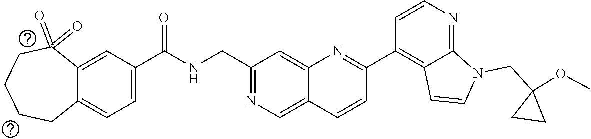 |
| 586 | 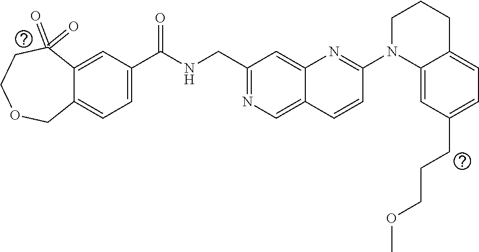 |
| 587 | 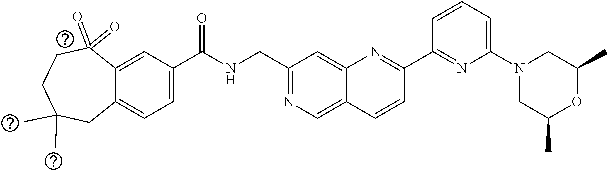 |
| 588 | 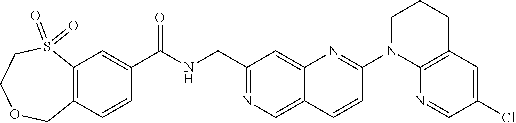 [SMILES: O=C(NCc1cc2nc(N3CCCc4cc(Cl)cnc43)ccc2cn1)c1ccc2c(c1)S(=O)(=O)CCOC2] |
| 589 | 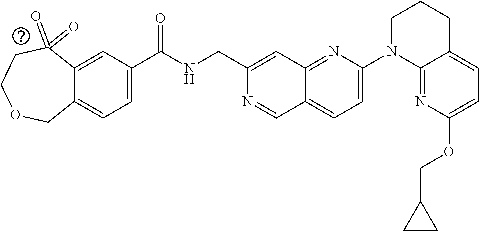 |
| 590 | 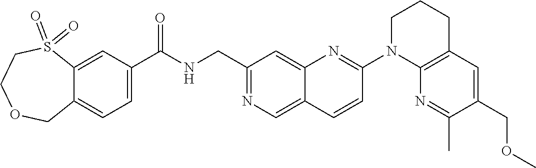 [SMILES: COCc1cc2c(nc1C)N(c1ccc3cnc(CNC(=O)c4ccc5c(c4)S(=O)(=O)CCOC5)cc3n1)CCC2] |
| 591 | 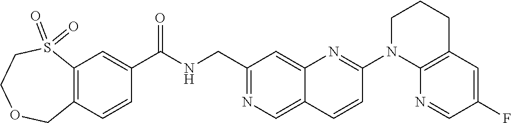 [SMILES: O=C(NCc1cc2nc(N3CCCc4cc(F)cnc43)ccc2cn1)c1ccc2c(c1)S(=O)(=O)CCOC2] |
| 592 | 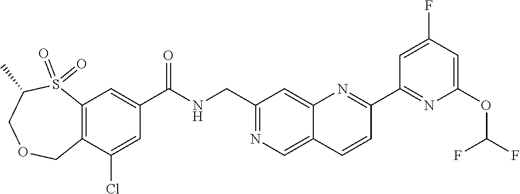 [SMILES: C[C@H]1COCc2c(Cl)cc(C(=O)NCc3cc4nc(-c5cc(F)cc(OC(F)F)n5)ccc4cn3)cc2S1(=O)=O] |
| 593 | 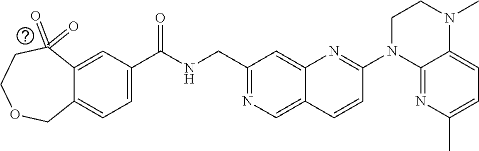 |
| 594 | 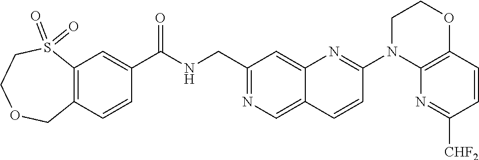 [SMILES: O=C(NCc1cc2nc(N3CCOc4ccc(C(F)F)nc43)ccc2cn1)c1ccc2c(c1)S(=O)(=O)CCOC2] |
| 595 | 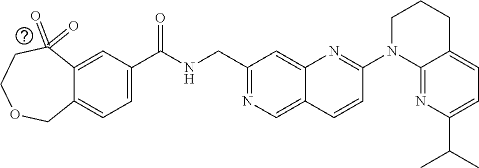 |
| 596 | 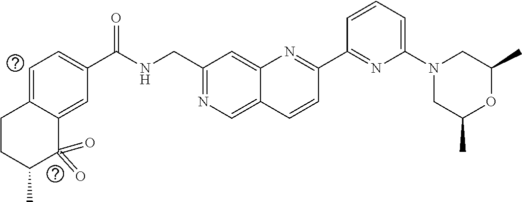 |
| 597 | 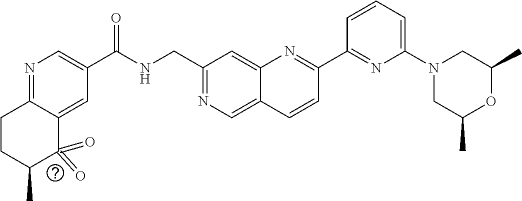 |
| 598 | 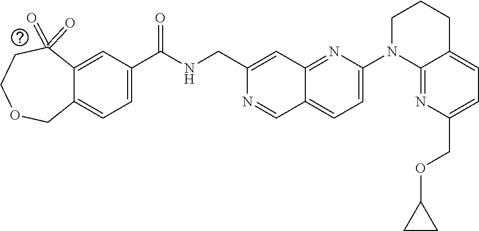 |
| 599 | 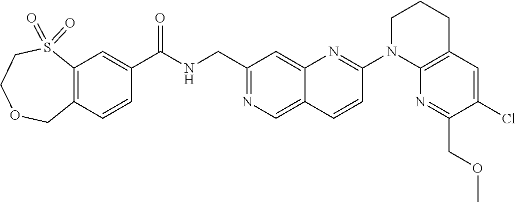 [SMILES: COCc1nc2c(cc1Cl)CCCN2c1ccc2cnc(CNC(=O)c3ccc4c(c3)S(=O)(=O)CCOC4)cc2n1] |
| | 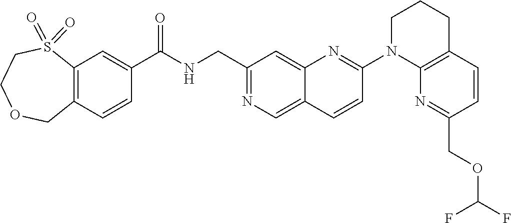 [SMILES: O=C(NCc1cc2nc(N3CCCc4ccc(COC(F)F)nc43)ccc2cn1)c1ccc2c(c1)S(=O)(=O)CCOC2] |
| 600 | 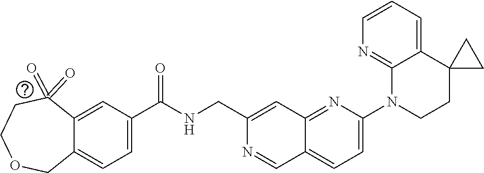 |
| 601 | 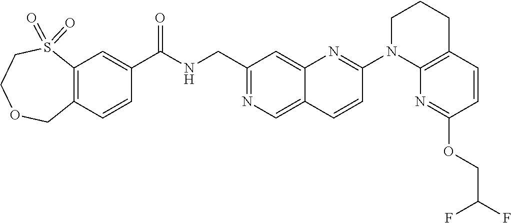 [SMILES: O=C(NCc1cc2nc(N3CCCc4ccc(OCC(F)F)nc43)ccc2cn1)c1ccc2c(c1)S(=O)(=O)CCOC2] |
| 602 | 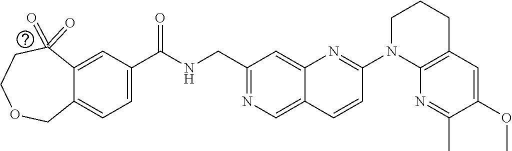 |
| 603 | 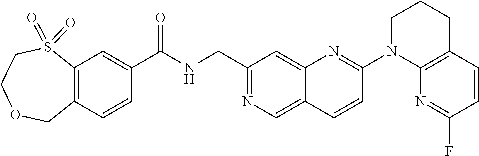 [SMILES: O=C(NCc1cc2nc(N3CCCc4ccc(F)nc43)ccc2cn1)c1ccc2c(c1)S(=O)(=O)CCOC2] |
| 604 | 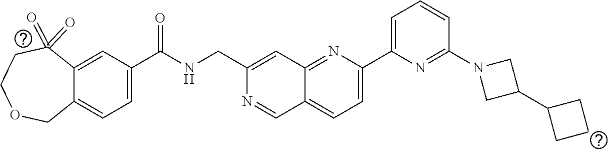 |
| 605 | 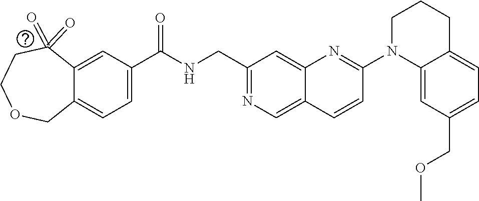 |
| 606 | 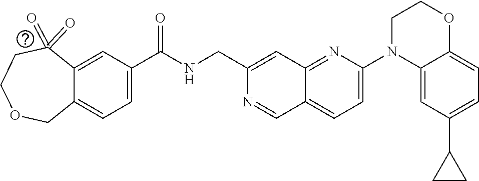 |
| 607 | 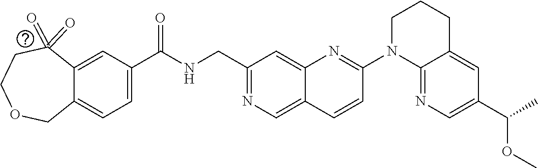 |
| 608 | 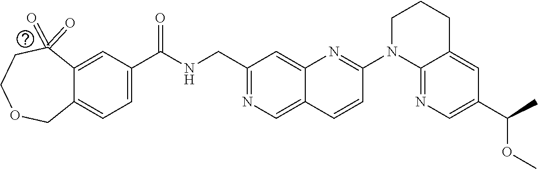 |
| 609 | 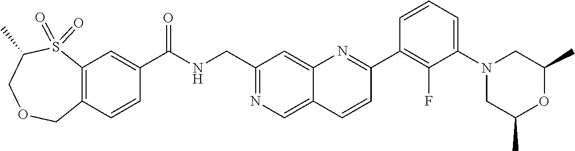 |
| 610 | 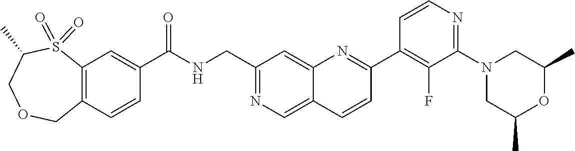 |
| 611 | 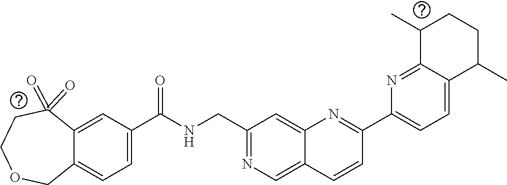 |
| 612 | 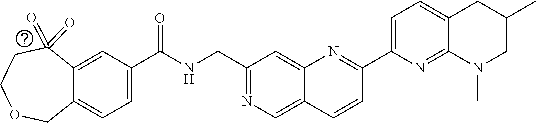 |
| 613 | 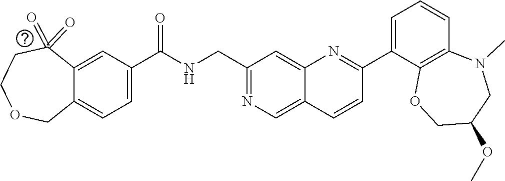 |
| 614 | 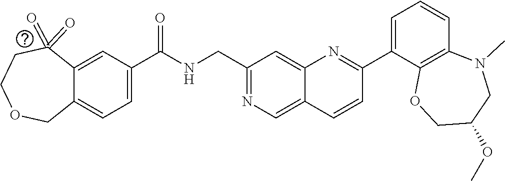 |
| 615 | 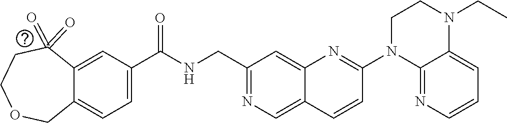 |
| 616 | 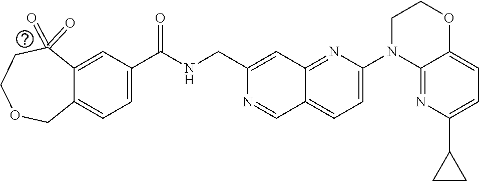 |
| 617 | 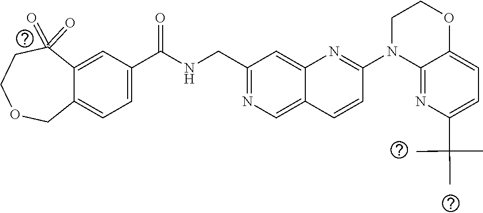 |
| 618 | 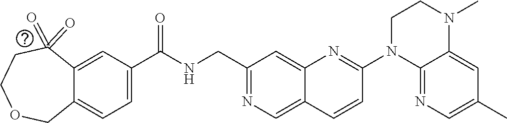 |
| 619 | 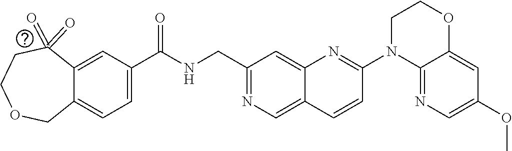 |
| 620 | 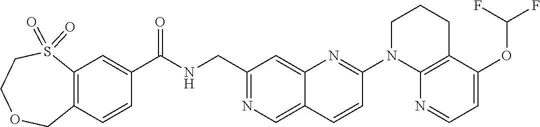 [SMILES: O=C(NCc1cc2nc(N3CCCc4c(OC(F)F)ccnc43)ccc2cn1)c1ccc2c(c1)S(=O)(=O)CCOC2] |
| 621 | 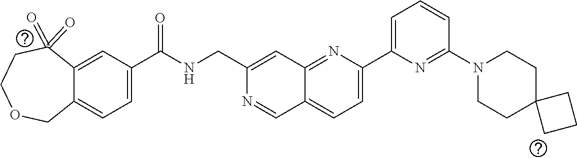 |
| 622 | 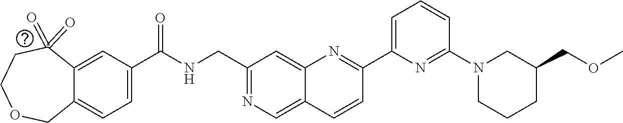 |
| 623 | 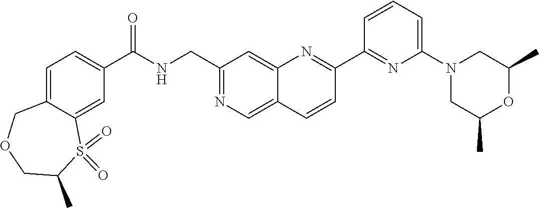 [SMILES: C[C@@H]1CN(c2cccc(-c3ccc4cnc(CNC(=O)c5ccc6c(c5)S(=O)(=O)[C@@H](C)COC6)cc4n3)n2)C[C@H](C)O1] |
| 624 | 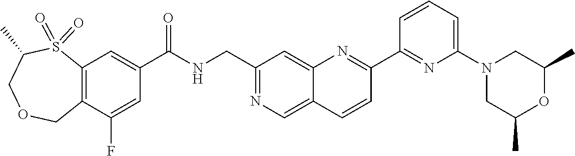 |
| 625 | 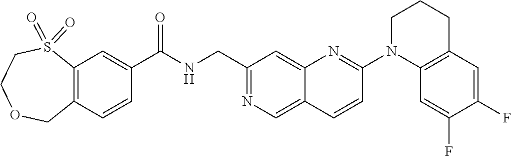 [SMILES: O=C(NCc1cc2nc(N3CCCc4cc(F)c(F)cc43)ccc2cn1)c1ccc2c(c1)S(=O)(=O)CCOC2] |
| 626 | 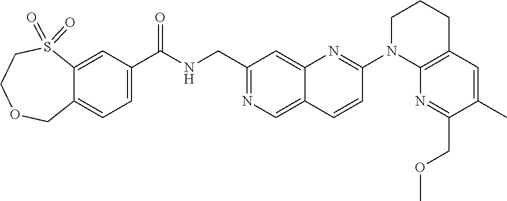 [SMILES: COCc1nc2c(cc1C)CCCN2c1ccc2cnc(CNC(=O)c3ccc4c(c3)S(=O)(=O)CCOC4)cc2n1] |
| 627 | 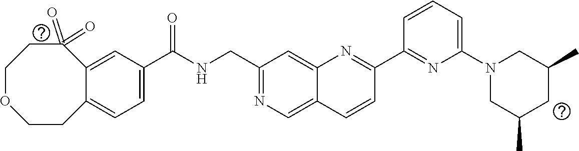 |
| 628 | 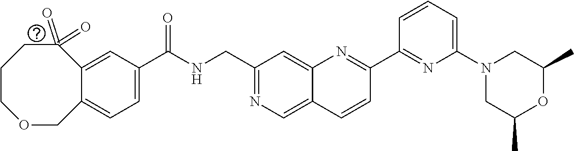 |
| 629 | 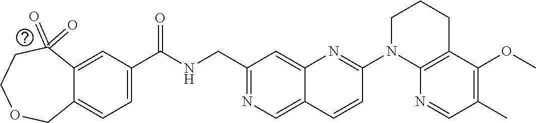 |
| 630 | 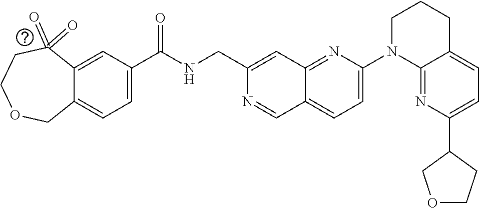 |
| 631 | 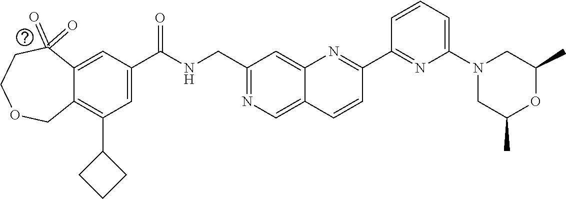 |
| 632 | 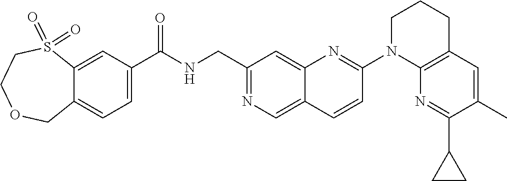 [SMILES: Cc1cc2c(nc1C1CC1)N(c1ccc3cnc(CNC(=O)c4ccc5c(c4)S(=O)(=O)CCOC5)cc3n1)CCC2] |
| 633 | 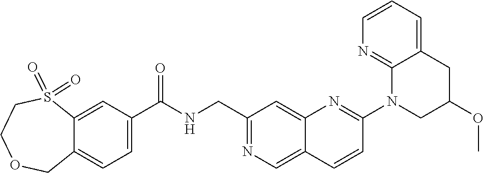 [SMILES: COC1Cc2cccnc2N(c2ccc3cnc(CNC(=O)c4ccc5c(c4)S(=O)(=O)CCOC5)cc3n2)C1] |
| 634 | 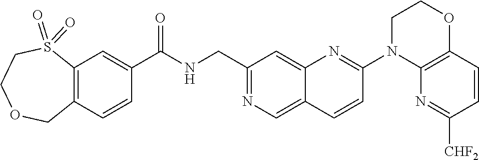 [SMILES: O=C(NCc1cc2nc(N3CCOc4ccc(C(F)F)nc43)ccc2cn1)c1ccc2c(c1)S(=O)(=O)CCOC2] |
| 635 | 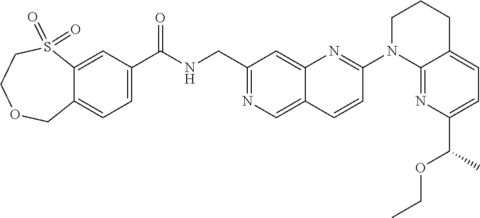 [SMILES: CCO[C@@H](C)c1ccc2c(n1)N(c1ccc3cnc(CNC(=O)c4ccc5c(c4)S(=O)(=O)CCOC5)cc3n1)CCC2] |
| 636 | 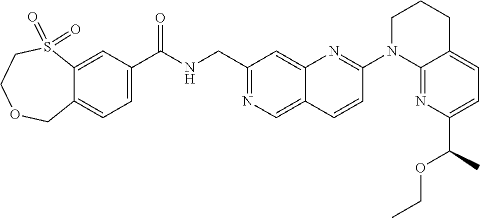 [SMILES: CCO[C@H](C)c1ccc2c(n1)N(c1ccc3cnc(CNC(=O)c4ccc5c(c4)S(=O)(=O)CCOC5)cc3n1)CCC2] |
| 637 | 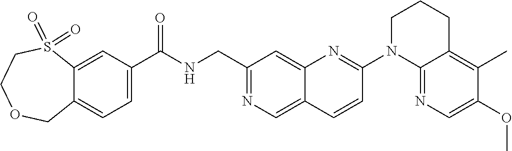 [SMILES: COc1cnc2c(c1C)CCCN2c1ccc2cnc(CNC(=O)c3ccc4c(c3)S(=O)(=O)CCOC4)cc2n1] |
| 638 | 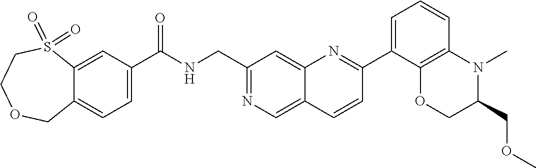 [SMILES: COC[C@H]1COc2c(-c3ccc4cnc(CNC(=O)c5ccc6c(c5)S(=O)(=O)CCOC6)cc4n3)cccc2N1C] |
| 639 | 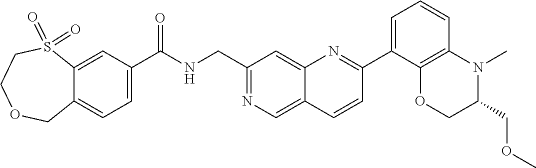 [SMILES: COC[C@@H]1COc2c(-c3ccc4cnc(CNC(=O)c5ccc6c(c5)S(=O)(=O)CCOC6)cc4n3)cccc2N1C] |
| 640 | 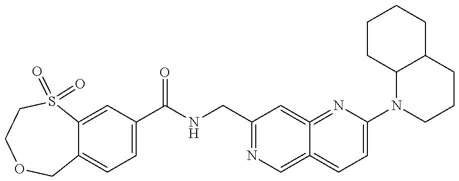 |
| 641 | 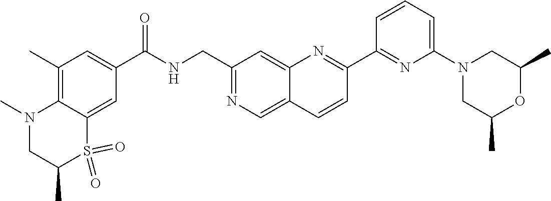 |
| 642 | 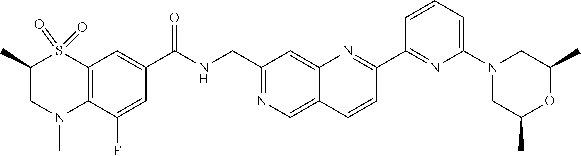 |
| 643 | 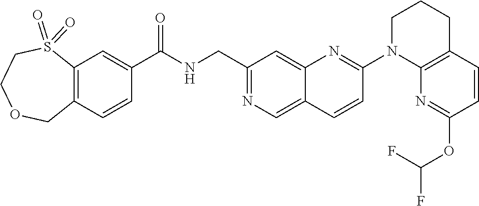 [SMILES: O=C(NCc1cc2nc(N3CCCc4ccc(OC(F)F)nc43)ccc2cn1)c1ccc2c(c1)S(=O)(=O)CCOC2] |
| 644 | 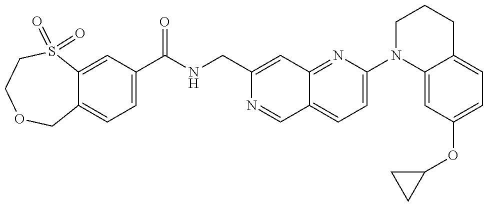 [SMILES: O=C(NCc1cc2nc(N3CCCc4ccc(OC5CC5)cc43)ccc2cn1)c1ccc2c(c1)S(=O)(=O)CCOC2] |
| 645 | 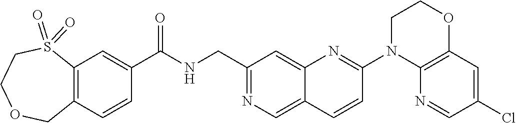 [SMILES: O=C(NCc1cc2nc(N3CCOc4cc(Cl)cnc43)ccc2cn1)c1ccc2c(c1)S(=O)(=O)CCOC2] |
| 646 | 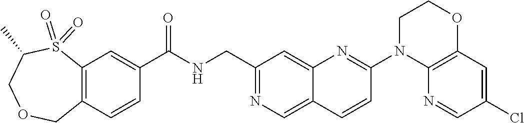 [SMILES: C[C@H]1COCc2ccc(C(=O)NCc3cc4nc(N5CCOc6cc(Cl)cnc65)ccc4cn3)cc2S1(=O)=O] |
| 647 | 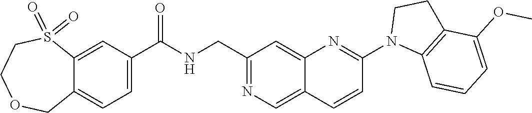 [SMILES: COc1cccc2c1CCN2c1ccc2cnc(CNC(=O)c3ccc4c(c3)S(=O)(=O)CCOC4)cc2n1] |
| 648 | 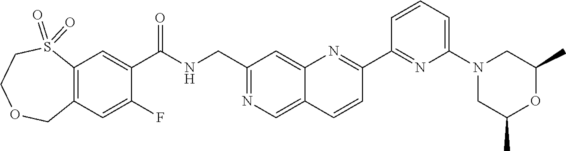 [SMILES: C[C@@H]1CN(c2cccc(-c3ccc4cnc(CNC(=O)c5cc6c(cc5F)COCCS6(=O)=O)cc4n3)n2)C[C@H](C)O1] |
| 649 | 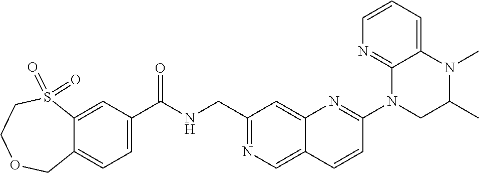 [SMILES: CC1CN(c2ccc3cnc(CNC(=O)c4ccc5c(c4)S(=O)(=O)CCOC5)cc3n2)c2ncccc2N1C] |
| 650 | 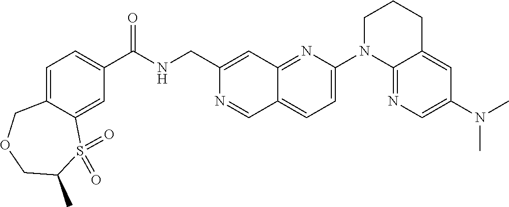 [SMILES: C[C@H]1COCc2ccc(C(=O)NCc3cc4nc(N5CCCc6cc(N(C)C)cnc65)ccc4cn3)cc2S1(=O)=O] |
| 651 | 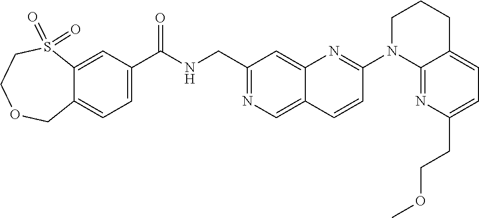 [SMILES: COCCc1ccc2c(n1)N(c1ccc3cnc(CNC(=O)c4ccc5c(c4)S(=O)(=O)CCOC5)cc3n1)CCC2] |
| 652 | 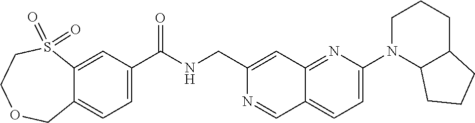 [SMILES: O=C(NCc1cc2nc(N3CCCC4CCCC43)ccc2cn1)c1ccc2c(c1)S(=O)(=O)CCOC2] |
| 653 | 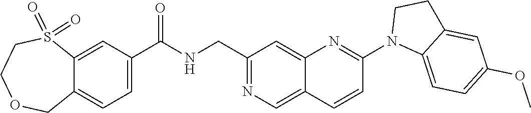 [SMILES: COc1ccc2c(c1)CCN2c1ccc2cnc(CNC(=O)c3ccc4c(c3)S(=O)(=O)CCOC4)cc2n1] |
| 654 | 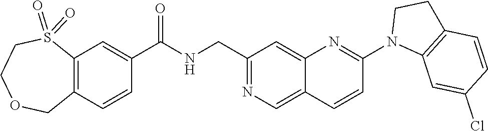 [SMILES: O=C(NCc1cc2nc(N3CCc4ccc(Cl)cc43)ccc2cn1)c1ccc2c(c1)S(=O)(=O)CCOC2] |
| 655 | 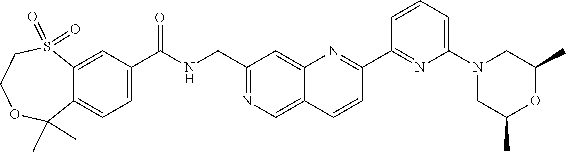 [SMILES: C[C@@H]1CN(c2cccc(-c3ccc4cnc(CNC(=O)c5ccc6c(c5)S(=O)(=O)CCOC6(C)C)cc4n3)n2)C[C@H](C)O1] |
| 656 | 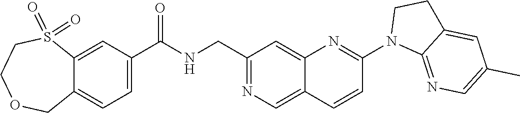 [SMILES: Cc1cnc2c(c1)CCN2c1ccc2cnc(CNC(=O)c3ccc4c(c3)S(=O)(=O)CCOC4)cc2n1] |
| 657 | 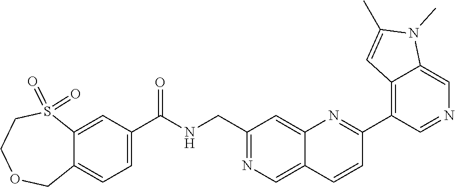 [SMILES: Cc1cc2c(-c3ccc4cnc(CNC(=O)c5ccc6c(c5)S(=O)(=O)CCOC6)cc4n3)cncc2n1C] |
| 658 | 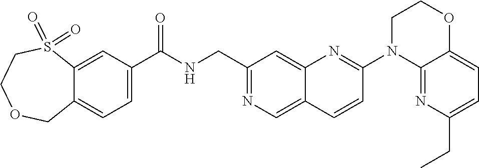 [SMILES: CCc1ccc2c(n1)N(c1ccc3cnc(CNC(=O)c4ccc5c(c4)S(=O)(=O)CCOC5)cc3n1)CCO2] |
| 659 | 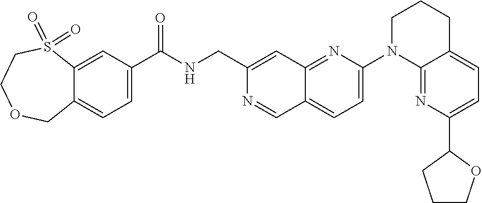 [SMILES: O=C(NCc1cc2nc(N3CCCc4ccc(C5CCCO5)nc43)ccc2cn1)c1ccc2c(c1)S(=O)(=O)CCOC2] |
| 660 | 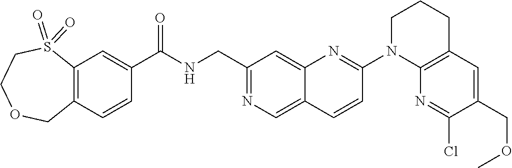 [SMILES: COCc1cc2c(nc1Cl)N(c1ccc3cnc(CNC(=O)c4ccc5c(c4)S(=O)(=O)CCOC5)cc3n1)CCC2] |
| 661 | 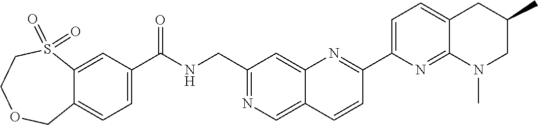 [SMILES: C[C@@H]1Cc2ccc(-c3ccc4cnc(CNC(=O)c5ccc6c(c5)S(=O)(=O)CCOC6)cc4n3)nc2N(C)C1] |
| 662 | 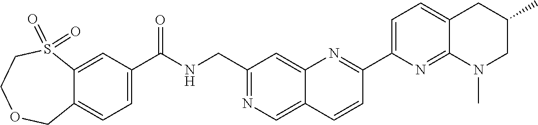 [SMILES: C[C@H]1Cc2ccc(-c3ccc4cnc(CNC(=O)c5ccc6c(c5)S(=O)(=O)CCOC6)cc4n3)nc2N(C)C1] |
| 663 | 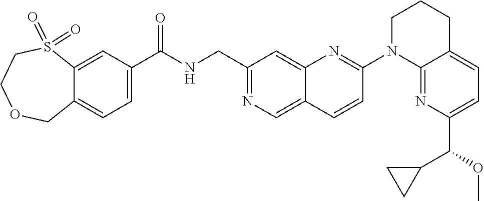 [SMILES: CO[C@@H](c1ccc2c(n1)N(c1ccc3cnc(CNC(=O)c4ccc5c(c4)S(=O)(=O)CCOC5)cc3n1)CCC2)C1CC1] |
| 664 | 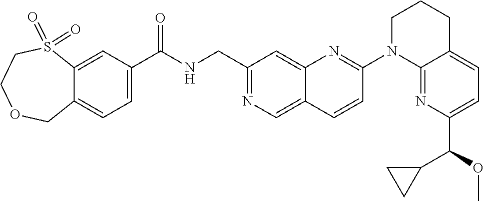 [SMILES: CO[C@H](c1ccc2c(n1)N(c1ccc3cnc(CNC(=O)c4ccc5c(c4)S(=O)(=O)CCOC5)cc3n1)CCC2)C1CC1] |
| 665 | 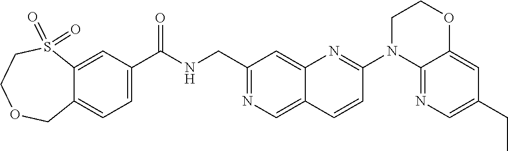 [SMILES: CCc1cnc2c(c1)OCCN2c1ccc2cnc(CNC(=O)c3ccc4c(c3)S(=O)(=O)CCOC4)cc2n1] |
| 666 | 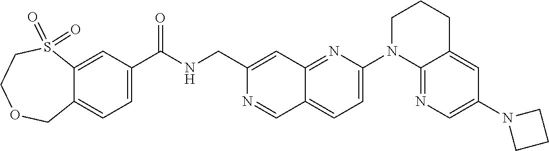 [SMILES: O=C(NCc1cc2nc(N3CCCc4cc(N5CCC5)cnc43)ccc2cn1)c1ccc2c(c1)S(=O)(=O)CCOC2] |
| 667 | 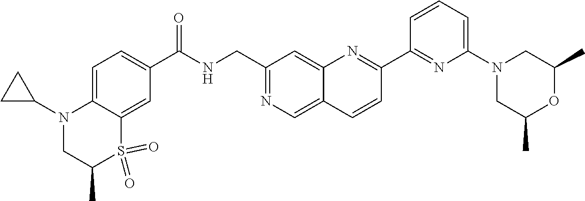 [SMILES: C[C@@H]1CN(c2cccc(-c3ccc4cnc(CNC(=O)c5ccc6c(c5)S(=O)(=O)[C@@H](C)CN6C5CC5)cc4n3)n2)C[C@H](C)O1] |
| 668 | 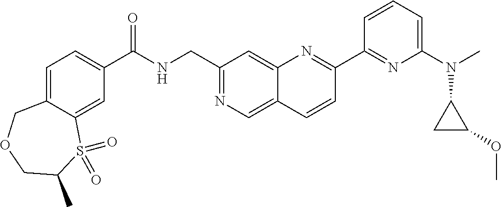 [SMILES: CO[C@@H]1C[C@@H]1N(C)c1cccc(-c2ccc3cnc(CNC(=O)c4ccc5c(c4)S(=O)(=O)[C@@H](C)COC5)cc3n2)n1] |
| 669 | 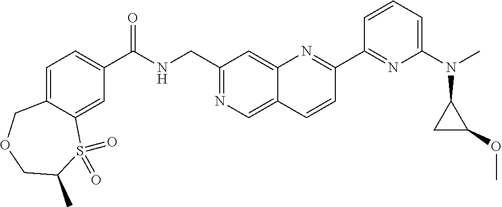 [SMILES: CO[C@H]1C[C@H]1N(C)c1cccc(-c2ccc3cnc(CNC(=O)c4ccc5c(c4)S(=O)(=O)[C@@H](C)COC5)cc3n2)n1] |
| 670 | 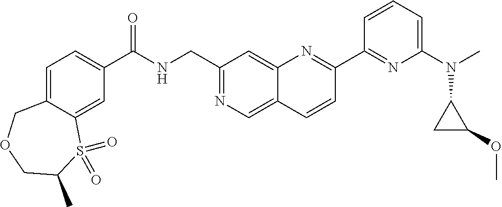 [SMILES: CO[C@H]1C[C@@H]1N(C)c1cccc(-c2ccc3cnc(CNC(=O)c4ccc5c(c4)S(=O)(=O)[C@@H](C)COC5)cc3n2)n1] |
| 671 | 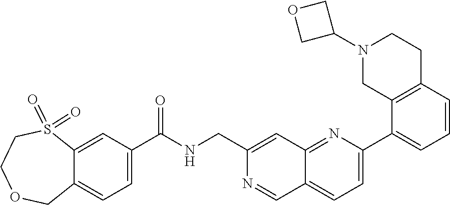 [SMILES: O=C(NCc1cc2nc(-c3cccc4c3CN(C3COC3)CC4)ccc2cn1)c1ccc2c(c1)S(=O)(=O)CCOC2] |
| 672 | 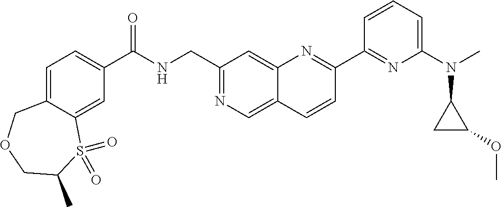 [SMILES: CO[C@@H]1C[C@H]1N(C)c1cccc(-c2ccc3cnc(CNC(=O)c4ccc5c(c4)S(=O)(=O)[C@@H](C)COC5)cc3n2)n1] |
| 673 | 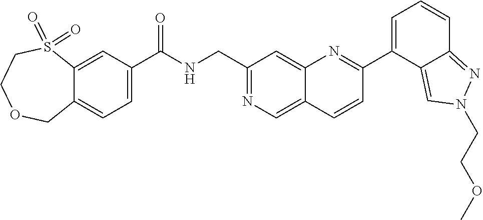 [SMILES: COCCn1cc2c(-c3ccc4cnc(CNC(=O)c5ccc6c(c5)S(=O)(=O)CCOC6)cc4n3)cccc2n1] |
| 674 | 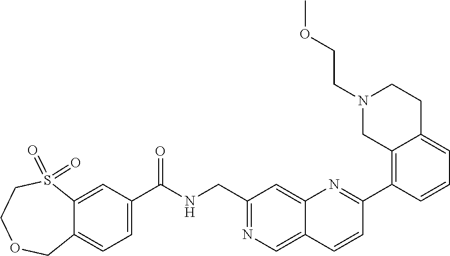 [SMILES: COCCN1CCc2cccc(-c3ccc4cnc(CNC(=O)c5ccc6c(c5)S(=O)(=O)CCOC6)cc4n3)c2C1] |
| 675 | 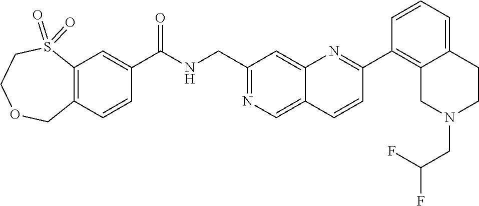 [SMILES: O=C(NCc1cc2nc(-c3cccc4c3CN(CC(F)F)CC4)ccc2cn1)c1ccc2c(c1)S(=O)(=O)CCOC2] |
| 676 | 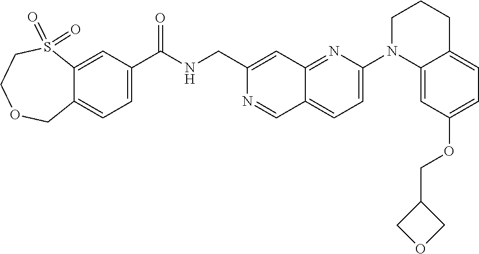 [SMILES: O=C(NCc1cc2nc(N3CCCc4ccc(OCC5COC5)cc43)ccc2cn1)c1ccc2c(c1)S(=O)(=O)CCOC2] |
| 677 | 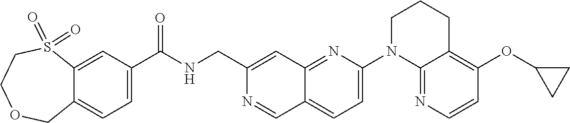 [SMILES: O=C(NCc1cc2nc(N3CCCc4c(OC5CC5)ccnc43)ccc2cn1)c1ccc2c(c1)S(=O)(=O)CCOC2] |
| 678 | 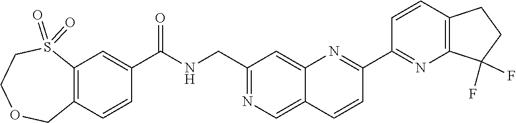 [SMILES: O=C(NCc1cc2nc(-c3ccc4c(n3)C(F)(F)CC4)ccc2cn1)c1ccc2c(c1)S(=O)(=O)CCOC2] |
| 679 | 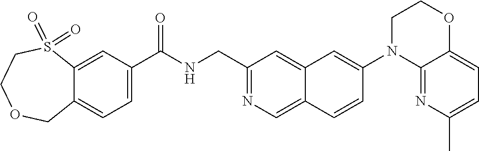 [SMILES: Cc1ccc2c(n1)N(c1ccc3cnc(CNC(=O)c4ccc5c(c4)S(=O)(=O)CCOC5)cc3c1)CCO2] |
| 680 | 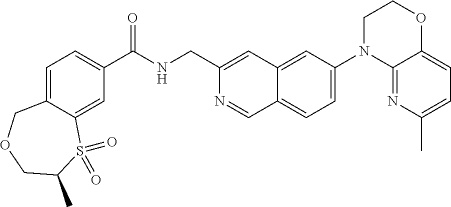 [SMILES: Cc1ccc2c(n1)N(c1ccc3cnc(CNC(=O)c4ccc5c(c4)S(=O)(=O)[C@@H](C)COC5)cc3c1)CCO2] |
| 681 | 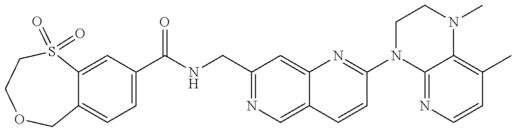 [SMILES: Cc1ccnc2c1N(C)CCN2c1ccc2cnc(CNC(=O)c3ccc4c(c3)S(=O)(=O)CCOC4)cc2n1] |
| 682 | 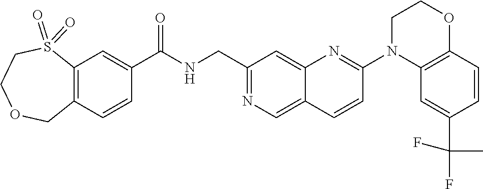 [SMILES: CC(F)(F)c1ccc2c(c1)N(c1ccc3cnc(CNC(=O)c4ccc5c(c4)S(=O)(=O)CCOC5)cc3n1)CCO2] |
| 683 | 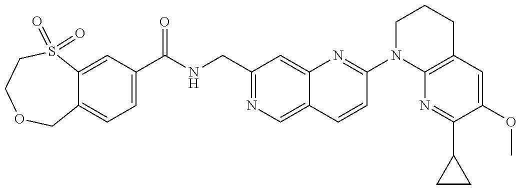 [SMILES: COc1cc2c(nc1C1CC1)N(c1ccc3cnc(CNC(=O)c4ccc5c(c4)S(=O)(=O)CCOC5)cc3n1)CCC2] |
| 684 | 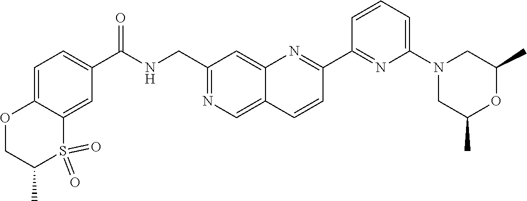 [SMILES: C[C@@H]1CN(c2cccc(-c3ccc4cnc(CNC(=O)c5ccc6c(c5)S(=O)(=O)[C@H](C)CO6)cc4n3)n2)C[C@H](C)O1] |
| 685 | 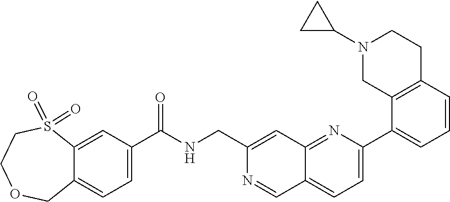 [SMILES: O=C(NCc1cc2nc(-c3cccc4c3CN(C3CC3)CC4)ccc2cn1)c1ccc2c(c1)S(=O)(=O)CCOC2] |
| 686 | 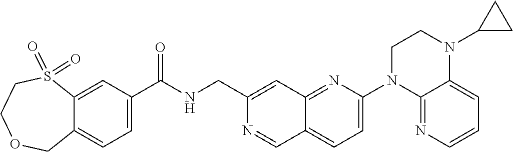 [SMILES: O=C(NCc1cc2nc(N3CCN(C4CC4)c4cccnc43)ccc2cn1)c1ccc2c(c1)S(=O)(=O)CCOC2] |
| 687 | 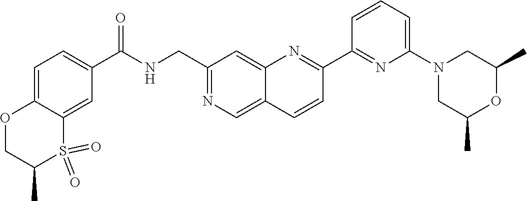 [SMILES: C[C@@H]1CN(c2cccc(-c3ccc4cnc(CNC(=O)c5ccc6c(c5)S(=O)(=O)[C@@H](C)CO6)cc4n3)n2)C[C@H](C)O1] |
| 688 | 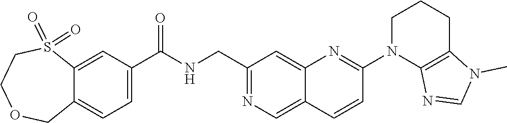 [SMILES: Cn1cnc2c1CCCN2c1ccc2cnc(CNC(=O)c3ccc4c(c3)S(=O)(=O)CCOC4)cc2n1] |
| 689 | 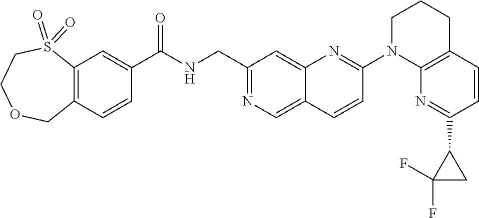 [SMILES: O=C(NCc1cc2nc(N3CCCc4ccc([C@@H]5CC5(F)F)nc43)ccc2cn1)c1ccc2c(c1)S(=O)(=O)CCOC2] |
| 690 | 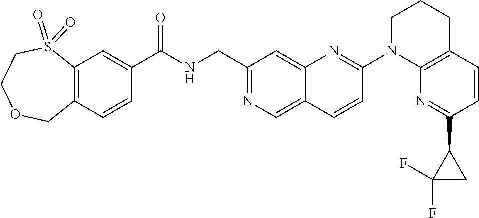 [SMILES: O=C(NCc1cc2nc(N3CCCc4ccc([C@H]5CC5(F)F)nc43)ccc2cn1)c1ccc2c(c1)S(=O)(=O)CCOC2] |
| 691 | 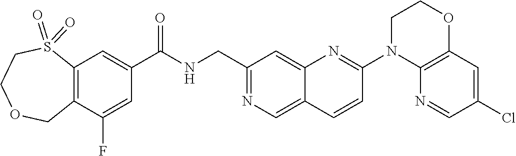 [SMILES: O=C(NCc1cc2nc(N3CCOc4cc(Cl)cnc43)ccc2cn1)c1cc(F)c2c(c1)S(=O)(=O)CCOC2] |
| 692 | 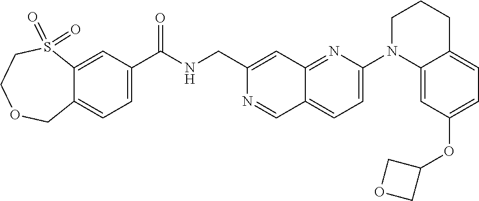 [SMILES: O=C(NCc1cc2nc(N3CCCc4ccc(OC5COC5)cc43)ccc2cn1)c1ccc2c(c1)S(=O)(=O)CCOC2] |
| 693 | 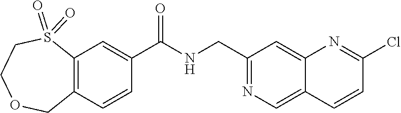 [SMILES: O=C(NCc1cc2nc(Cl)ccc2cn1)c1ccc2c(c1)S(=O)(=O)CCOC2] |
| 694 | 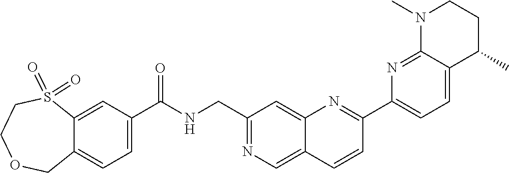 [SMILES: C[C@H]1CCN(C)c2nc(-c3ccc4cnc(CNC(=O)c5ccc6c(c5)S(=O)(=O)CCOC6)cc4n3)ccc21] |
| 695 | 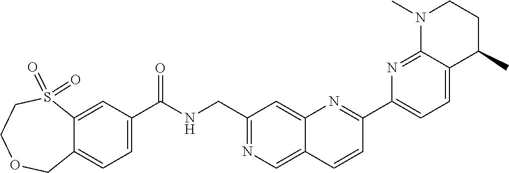 [SMILES: C[C@@H]1CCN(C)c2nc(-c3ccc4cnc(CNC(=O)c5ccc6c(c5)S(=O)(=O)CCOC6)cc4n3)ccc21] |
| 696 | 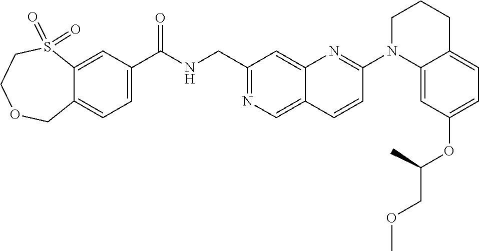 [SMILES: COC[C@@H](C)Oc1ccc2c(c1)N(c1ccc3cnc(CNC(=O)c4ccc5c(c4)S(=O)(=O)CCOC5)cc3n1)CCC2] |
| 697 | 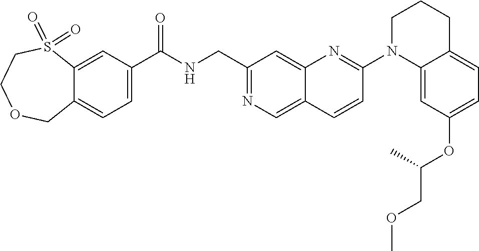 [SMILES: COC[C@H](C)Oc1ccc2c(c1)N(c1ccc3cnc(CNC(=O)c4ccc5c(c4)S(=O)(=O)CCOC5)cc3n1)CCC2] |
| 698 | 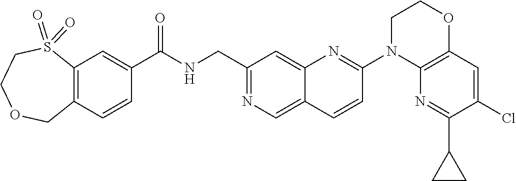 [SMILES: O=C(NCc1cc2nc(N3CCOc4cc(Cl)c(C5CC5)nc43)ccc2cn1)c1ccc2c(c1)S(=O)(=O)CCOC2] |
| 699 | 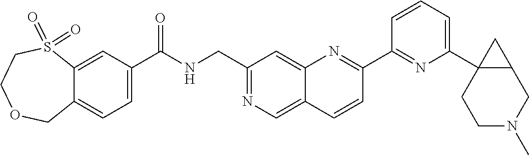 [SMILES: CN1CCC2(c3cccc(-c4ccc5cnc(CNC(=O)c6ccc7c(c6)S(=O)(=O)CCOC7)cc5n4)n3)CC2C1] |
| 700 | 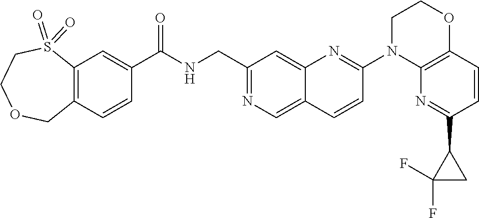 [SMILES: O=C(NCc1cc2nc(N3CCOc4ccc([C@H]5CC5(F)F)nc43)ccc2cn1)c1ccc2c(c1)S(=O)(=O)CCOC2] |
| 701 | 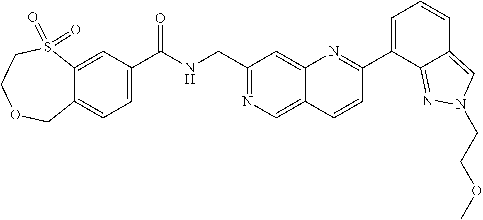 [SMILES: COCCn1cc2cccc(-c3ccc4cnc(CNC(=O)c5ccc6c(c5)S(=O)(=O)CCOC6)cc4n3)c2n1] |
| 702 | 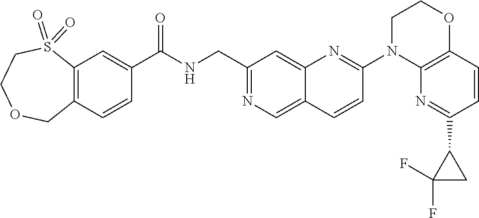 [SMILES: O=C(NCc1cc2nc(N3CCOc4ccc([C@@H]5CC5(F)F)nc43)ccc2cn1)c1ccc2c(c1)S(=O)(=O)CCOC2] |
| 703 | 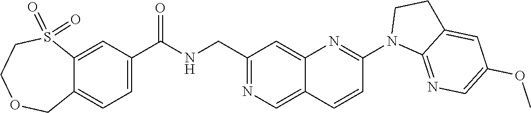 [SMILES: COc1cnc2c(c1)CCN2c1ccc2cnc(CNC(=O)c3ccc4c(c3)S(=O)(=O)CCOC4)cc2n1] |
| 704 | 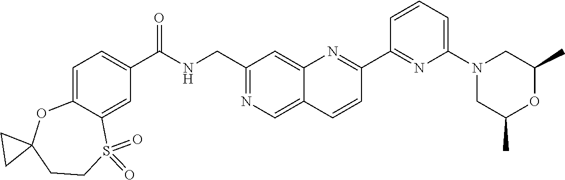 [SMILES: C[C@@H]1CN(c2cccc(-c3ccc4cnc(CNC(=O)c5ccc6c(c5)S(=O)(=O)CCC5(CC5)O6)cc4n3)n2)C[C@H](C)O1] |
| 705 | 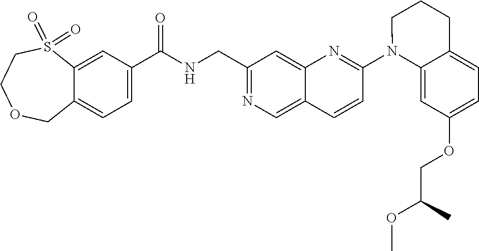 [SMILES: CO[C@H](C)COc1ccc2c(c1)N(c1ccc3cnc(CNC(=O)c4ccc5c(c4)S(=O)(=O)CCOC5)cc3n1)CCC2] |
| 706 | 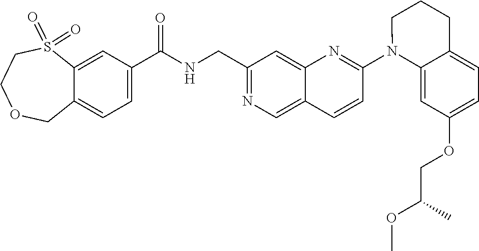 [SMILES: CO[C@@H](C)COc1ccc2c(c1)N(c1ccc3cnc(CNC(=O)c4ccc5c(c4)S(=O)(=O)CCOC5)cc3n1)CCC2] |
| 707 | 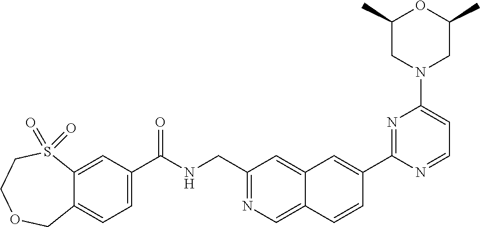 [SMILES: C[C@@H]1CN(c2ccnc(-c3ccc4cnc(CNC(=O)c5ccc6c(c5)S(=O)(=O)CCOC6)cc4c3)n2)C[C@H](C)O1] |
| 708 | 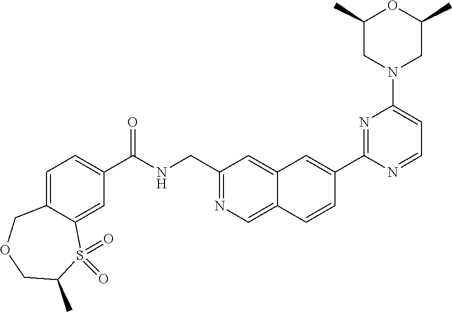 [SMILES: C[C@@H]1CN(c2ccnc(-c3ccc4cnc(CNC(=O)c5ccc6c(c5)S(=O)(=O)[C@@H](C)COC6)cc4c3)n2)C[C@H](C)O1] |
| 709 | 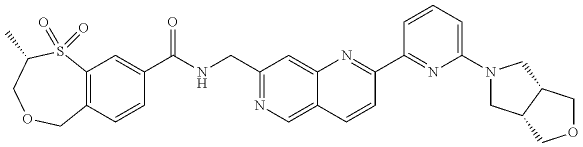 [SMILES: C[C@H]1COCc2ccc(C(=O)NCc3cc4nc(-c5cccc(N6C[C@H]7COC[C@H]7C6)n5)ccc4cn3)cc2S1(=O)=O] |
| 710 | 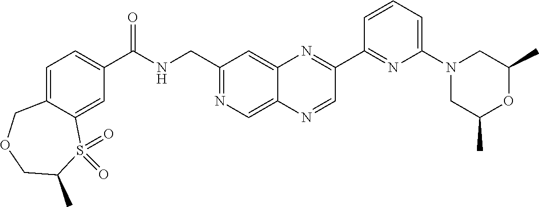 [SMILES: C[C@@H]1CN(c2cccc(-c3cnc4cnc(CNC(=O)c5ccc6c(c5)S(=O)(=O)[C@@H](C)COC6)cc4n3)n2)C[C@H](C)O1] |
| 711 | 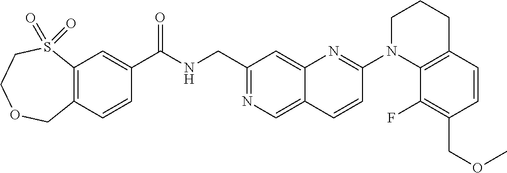 [SMILES: COCc1ccc2c(c1F)N(c1ccc3cnc(CNC(=O)c4ccc5c(c4)S(=O)(=O)CCOC5)cc3n1)CCC2] |
| 712 | 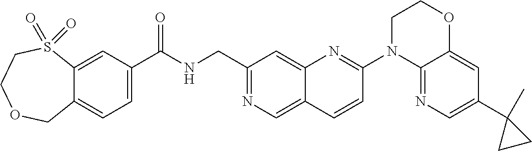 [SMILES: CC1(c2cnc3c(c2)OCCN3c2ccc3cnc(CNC(=O)c4ccc5c(c4)S(=O)(=O)CCOC5)cc3n2)CC1] |
| 713 | 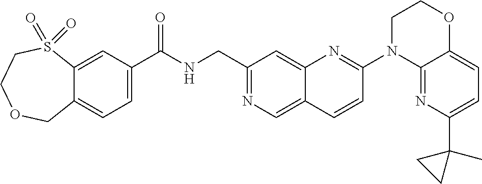 [SMILES: CC1(c2ccc3c(n2)N(c2ccc4cnc(CNC(=O)c5ccc6c(c5)S(=O)(=O)CCOC6)cc4n2)CCO3)CC1] |
| 714 | 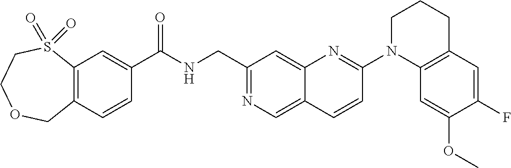 [SMILES: COc1cc2c(cc1F)CCCN2c1ccc2cnc(CNC(=O)c3ccc4c(c3)S(=O)(=O)CCOC4)cc2n1] |
| 715 | 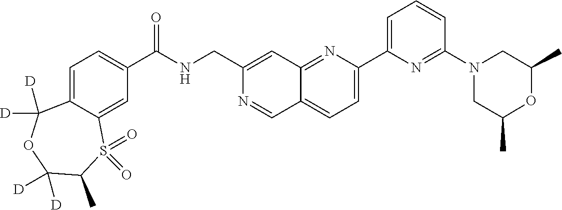 [SMILES: [2H]C1([2H])OC([2H])([2H])[C@H](C)S(=O)(=O)c2cc(C(=O)NCc3cc4nc(-c5cccc(N6C[C@@H](C)O[C@@H](C)C6)n5)ccc4cn3)ccc21] |
| 716 | 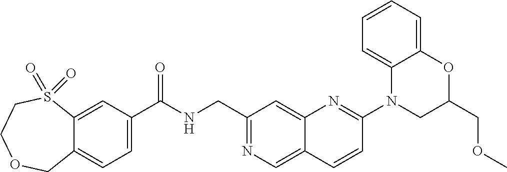 [SMILES: COCC1CN(c2ccc3cnc(CNC(=O)c4ccc5c(c4)S(=O)(=O)CCOC5)cc3n2)c2ccccc2O1] |
| 717 | 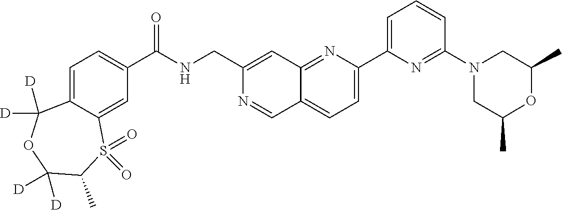 [SMILES: [2H]C1([2H])OC([2H])([2H])[C@@H](C)S(=O)(=O)c2cc(C(=O)NCc3cc4nc(-c5cccc(N6C[C@@H](C)O[C@@H](C)C6)n5)ccc4cn3)ccc21] |
| 718 | 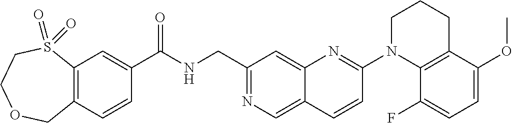 [SMILES: COc1ccc(F)c2c1CCCN2c1ccc2cnc(CNC(=O)c3ccc4c(c3)S(=O)(=O)CCOC4)cc2n1] |
| 719 | 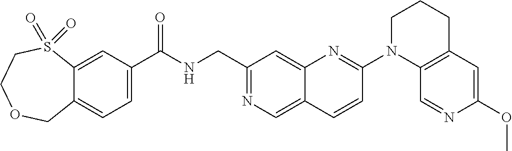 [SMILES: COc1cc2c(cn1)N(c1ccc3cnc(CNC(=O)c4ccc5c(c4)S(=O)(=O)CCOC5)cc3n1)CCC2] |
| 720 | 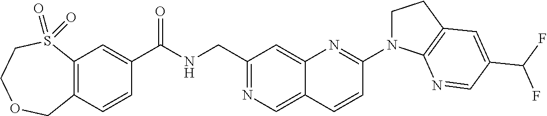 [SMILES: O=C(NCc1cc2nc(N3CCc4cc(C(F)F)cnc43)ccc2cn1)c1ccc2c(c1)S(=O)(=O)CCOC2] |
| 721 | 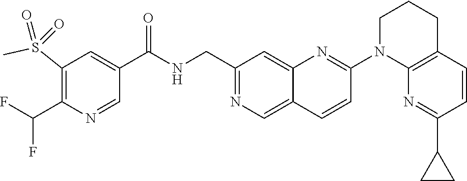 [SMILES: CS(=O)(=O)c1cc(C(=O)NCc2cc3nc(N4CCCc5ccc(C6CC6)nc54)ccc3cn2)cnc1C(F)F] |
| 722 | 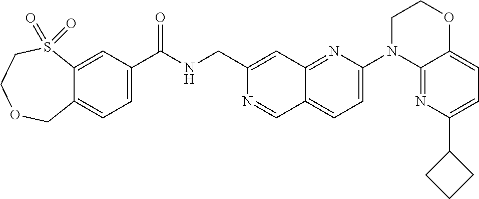 [SMILES: O=C(NCc1cc2nc(N3CCOc4ccc(C5CCC5)nc43)ccc2cn1)c1ccc2c(c1)S(=O)(=O)CCOC2] |
| 723 | 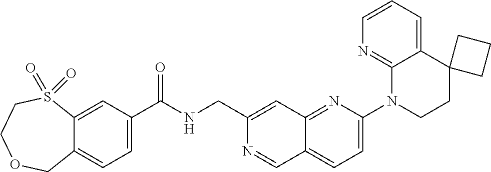 [SMILES: O=C(NCc1cc2nc(N3CCC4(CCC4)c4cccnc43)ccc2cn1)c1ccc2c(c1)S(=O)(=O)CCOC2] |
| 724 | 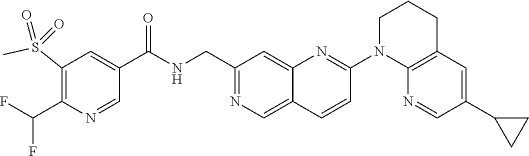 [SMILES: CS(=O)(=O)c1cc(C(=O)NCc2cc3nc(N4CCCc5cc(C6CC6)cnc54)ccc3cn2)cnc1C(F)F] |
| 725 | 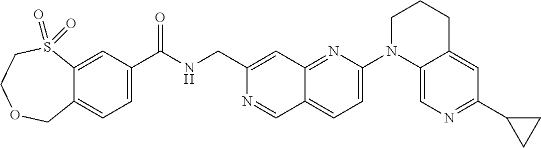 [SMILES: O=C(NCc1cc2nc(N3CCCc4cc(C5CC5)ncc43)ccc2cn1)c1ccc2c(c1)S(=O)(=O)CCOC2] |
| 726 | 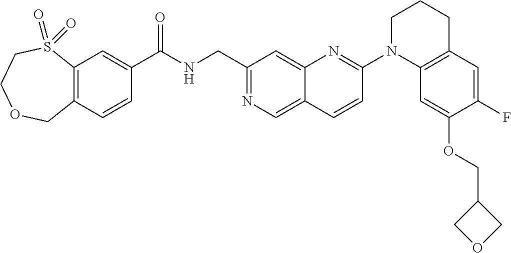 [SMILES: O=C(NCc1cc2nc(N3CCCc4cc(F)c(OCC5COC5)cc43)ccc2cn1)c1ccc2c(c1)S(=O)(=O)CCOC2] |
| 727 | 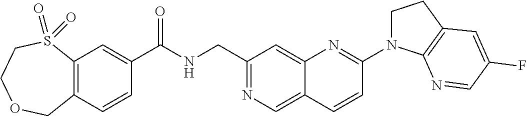 [SMILES: O=C(NCc1cc2nc(N3CCc4cc(F)cnc43)ccc2cn1)c1ccc2c(c1)S(=O)(=O)CCOC2] |
| 728 | 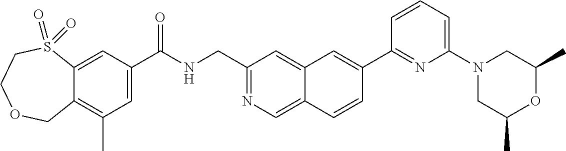 [SMILES: Cc1cc(C(=O)NCc2cc3cc(-c4cccc(N5C[C@@H](C)O[C@@H](C)C5)n4)ccc3cn2)cc2c1COCCS2(=O)=O] |
| 729 | 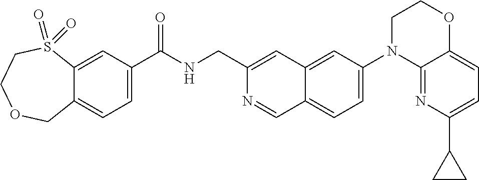 [SMILES: O=C(NCc1cc2cc(N3CCOc4ccc(C5CC5)nc43)ccc2cn1)c1ccc2c(c1)S(=O)(=O)CCOC2] |
| 730 | 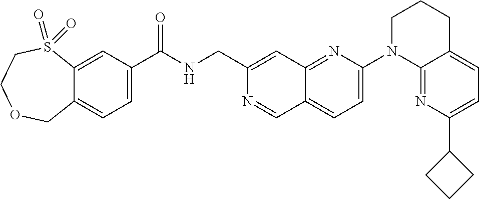 [SMILES: O=C(NCc1cc2nc(N3CCCc4ccc(C5CCC5)nc43)ccc2cn1)c1ccc2c(c1)S(=O)(=O)CCOC2] |
| 731 | 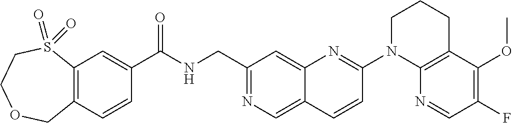 [SMILES: COc1c(F)cnc2c1CCCN2c1ccc2cnc(CNC(=O)c3ccc4c(c3)S(=O)(=O)CCOC4)cc2n1] |
| 732 | 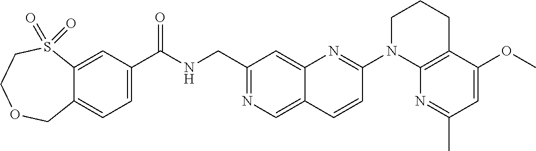 [SMILES: COc1cc(C)nc2c1CCCN2c1ccc2cnc(CNC(=O)c3ccc4c(c3)S(=O)(=O)CCOC4)cc2n1] |
| 733 | 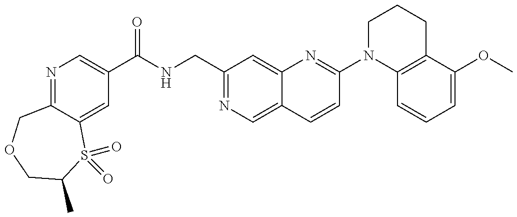 [SMILES: COc1cccc2c1CCCN2c1ccc2cnc(CNC(=O)c3cnc4c(c3)S(=O)(=O)[C@@H](C)COC4)cc2n1] |
| 734 | 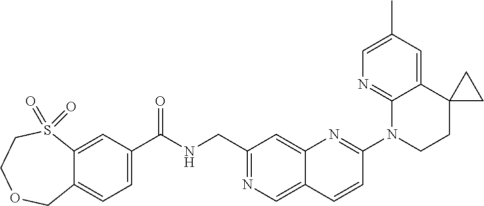 [SMILES: Cc1cnc2c(c1)C1(CCN2c2ccc3cnc(CNC(=O)c4ccc5c(c4)S(=O)(=O)CCOC5)cc3n2)CC1] |
| 735 | 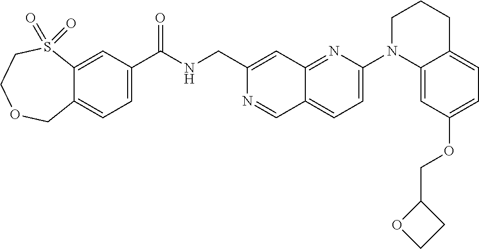 [SMILES: O=C(NCc1cc2nc(N3CCCc4ccc(OCC5CCO5)cc43)ccc2cn1)c1ccc2c(c1)S(=O)(=O)CCOC2] |
| 736 | 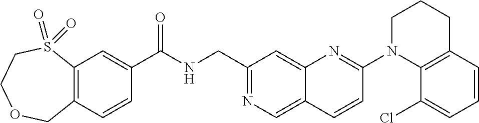 [SMILES: O=C(NCc1cc2nc(N3CCCc4cccc(Cl)c43)ccc2cn1)c1ccc2c(c1)S(=O)(=O)CCOC2] |
| 737 | 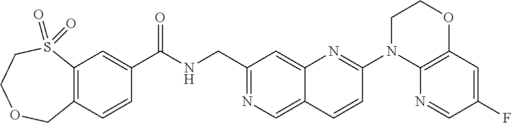 [SMILES: O=C(NCc1cc2nc(N3CCOc4cc(F)cnc43)ccc2cn1)c1ccc2c(c1)S(=O)(=O)CCOC2] |
| 738 | 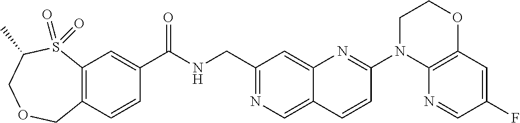 [SMILES: C[C@H]1COCc2ccc(C(=O)NCc3cc4nc(N5CCOc6cc(F)cnc65)ccc4cn3)cc2S1(=O)=O] |
| 739 | 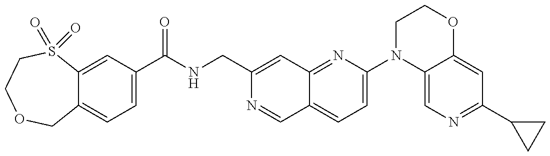 [SMILES: O=C(NCc1cc2nc(N3CCOc4cc(C5CC5)ncc43)ccc2cn1)c1ccc2c(c1)S(=O)(=O)CCOC2] |
| 740 | 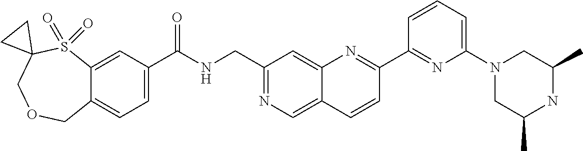 [SMILES: C[C@@H]1CN(c2cccc(-c3ccc4cnc(CNC(=O)c5ccc6c(c5)S(=O)(=O)C5(CC5)COC6)cc4n3)n2)C[C@H](C)N1] |
| 741 | 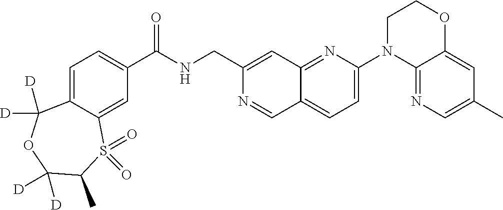 [SMILES: [2H]C1([2H])OC([2H])([2H])[C@H](C)S(=O)(=O)c2cc(C(=O)NCc3cc4nc(N5CCOc6cc(C)cnc65)ccc4cn3)ccc21] |
| 742 | 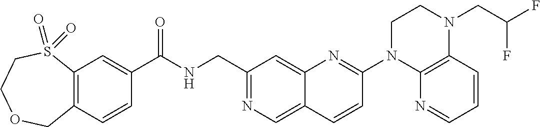 [SMILES: O=C(NCc1cc2nc(N3CCN(CC(F)F)c4cccnc43)ccc2cn1)c1ccc2c(c1)S(=O)(=O)CCOC2] |
| 743 | 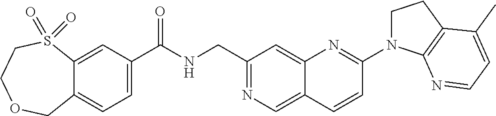 [SMILES: Cc1ccnc2c1CCN2c1ccc2cnc(CNC(=O)c3ccc4c(c3)S(=O)(=O)CCOC4)cc2n1] |
| 744 | 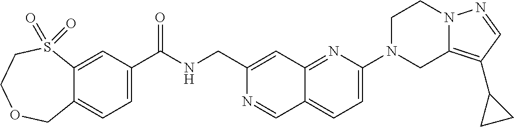 [SMILES: O=C(NCc1cc2nc(N3CCn4ncc(C5CC5)c4C3)ccc2cn1)c1ccc2c(c1)S(=O)(=O)CCOC2] |
| 745 | 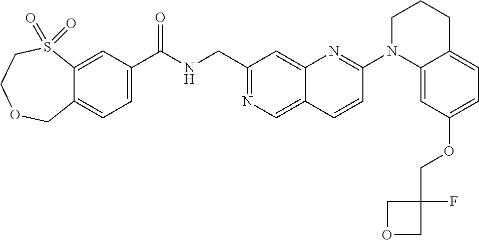 [SMILES: O=C(NCc1cc2nc(N3CCCc4ccc(OCC5(F)COC5)cc43)ccc2cn1)c1ccc2c(c1)S(=O)(=O)CCOC2] |
| 746 | 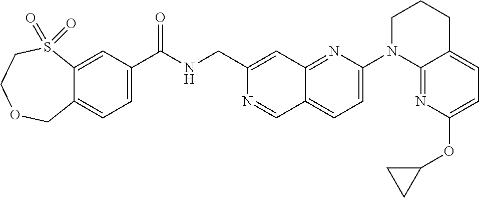 [SMILES: O=C(NCc1cc2nc(N3CCCc4ccc(OC5CC5)nc43)ccc2cn1)c1ccc2c(c1)S(=O)(=O)CCOC2] |
| 747 | 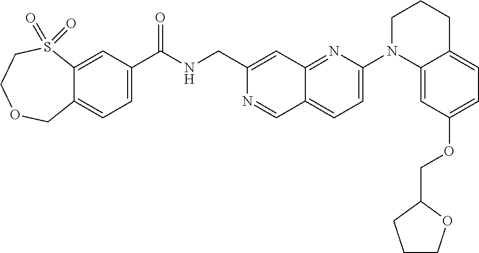 [SMILES: O=C(NCc1cc2nc(N3CCCc4ccc(OCC5CCCO5)cc43)ccc2cn1)c1ccc2c(c1)S(=O)(=O)CCOC2] |
| 748 | 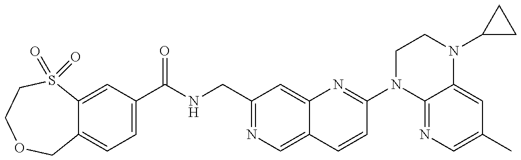 [SMILES: Cc1cnc2c(c1)N(C1CC1)CCN2c1ccc2cnc(CNC(=O)c3ccc4c(c3)S(=O)(=O)CCOC4)cc2n1] |
| 749 | 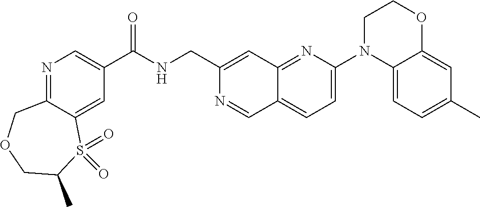 [SMILES: Cc1ccc2c(c1)OCCN2c1ccc2cnc(CNC(=O)c3cnc4c(c3)S(=O)(=O)[C@@H](C)COC4)cc2n1] |
| 750 | 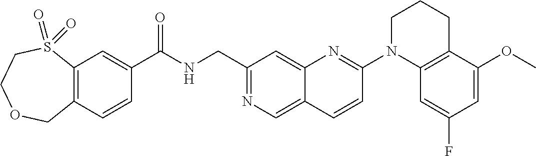 [SMILES: COc1cc(F)cc2c1CCCN2c1ccc2cnc(CNC(=O)c3ccc4c(c3)S(=O)(=O)CCOC4)cc2n1] |
| 751 | 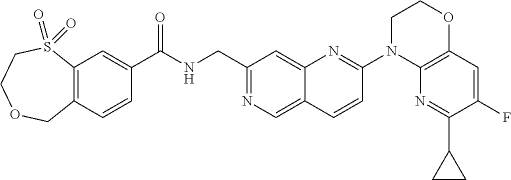 [SMILES: O=C(NCc1cc2nc(N3CCOc4cc(F)c(C5CC5)nc43)ccc2cn1)c1ccc2c(c1)S(=O)(=O)CCOC2] |
| 752 | 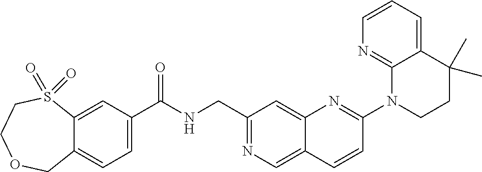 [SMILES: CC1(C)CCN(c2ccc3cnc(CNC(=O)c4ccc5c(c4)S(=O)(=O)CCOC5)cc3n2)c2ncccc21] |
| 753 | 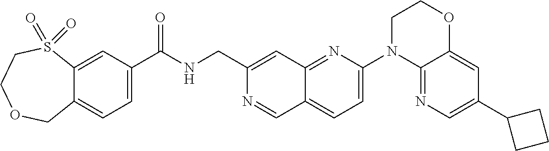 [SMILES: O=C(NCc1cc2nc(N3CCOc4cc(C5CCC5)cnc43)ccc2cn1)c1ccc2c(c1)S(=O)(=O)CCOC2] |
| 754 | 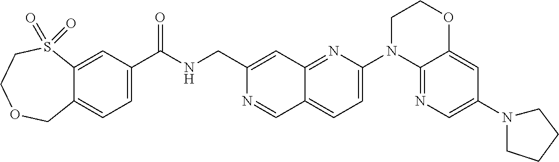 [SMILES: O=C(NCc1cc2nc(N3CCOc4cc(N5CCCC5)cnc43)ccc2cn1)c1ccc2c(c1)S(=O)(=O)CCOC2] |
| 755 | 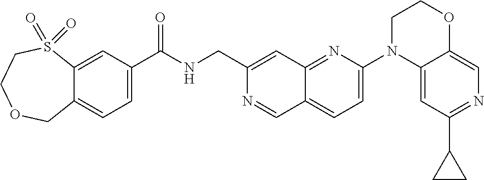 [SMILES: O=C(NCc1cc2nc(N3CCOc4cnc(C5CC5)cc43)ccc2cn1)c1ccc2c(c1)S(=O)(=O)CCOC2] |
| 756 | 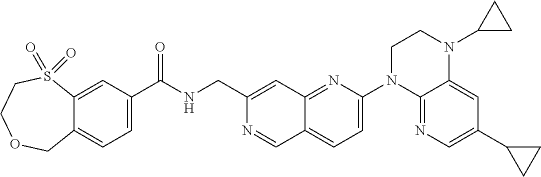 [SMILES: O=C(NCc1cc2nc(N3CCN(C4CC4)c4cc(C5CC5)cnc43)ccc2cn1)c1ccc2c(c1)S(=O)(=O)CCOC2] |
| 757 | 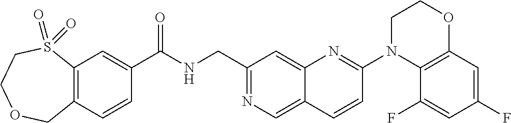 [SMILES: O=C(NCc1cc2nc(N3CCOc4cc(F)cc(F)c43)ccc2cn1)c1ccc2c(c1)S(=O)(=O)CCOC2] |
| 758 | 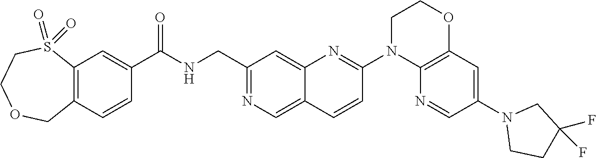 [SMILES: O=C(NCc1cc2nc(N3CCOc4cc(N5CCC(F)(F)C5)cnc43)ccc2cn1)c1ccc2c(c1)S(=O)(=O)CCOC2] |
| 759 | 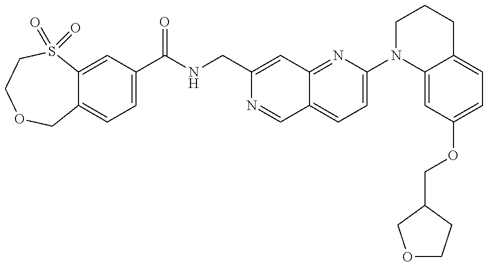 [SMILES: O=C(NCc1cc2nc(N3CCCc4ccc(OCC5CCOC5)cc43)ccc2cn1)c1ccc2c(c1)S(=O)(=O)CCOC2] |
| 760 | 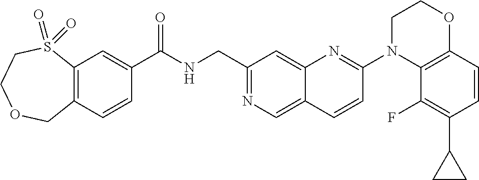 [SMILES: O=C(NCc1cc2nc(N3CCOc4ccc(C5CC5)c(F)c43)ccc2cn1)c1ccc2c(c1)S(=O)(=O)CCOC2] |
| 761 | 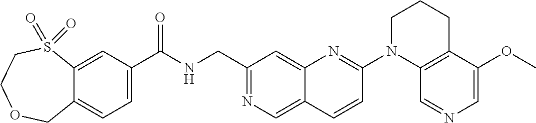 [SMILES: COc1cncc2c1CCCN2c1ccc2cnc(CNC(=O)c3ccc4c(c3)S(=O)(=O)CCOC4)cc2n1] |
| 762 | 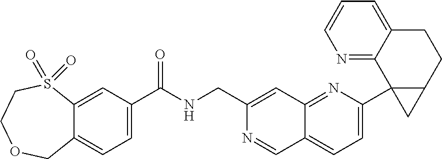 [SMILES: O=C(NCc1cc2nc(C34CC3CCc3cccnc34)ccc2cn1)c1ccc2c(c1)S(=O)(=O)CCOC2] |
| 763 | 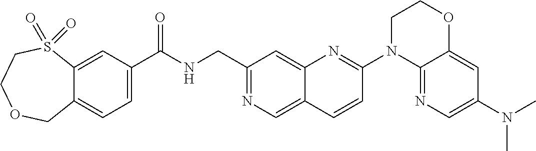 [SMILES: CN(C)c1cnc2c(c1)OCCN2c1ccc2cnc(CNC(=O)c3ccc4c(c3)S(=O)(=O)CCOC4)cc2n1] |
| 764 | 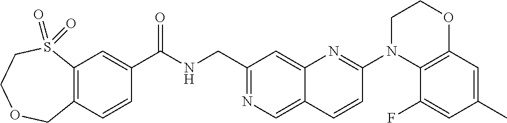 [SMILES: Cc1cc(F)c2c(c1)OCCN2c1ccc2cnc(CNC(=O)c3ccc4c(c3)S(=O)(=O)CCOC4)cc2n1] |
| 765 | 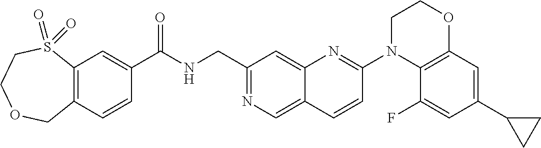 [SMILES: O=C(NCc1cc2nc(N3CCOc4cc(C5CC5)cc(F)c43)ccc2cn1)c1ccc2c(c1)S(=O)(=O)CCOC2] |
| 766 | 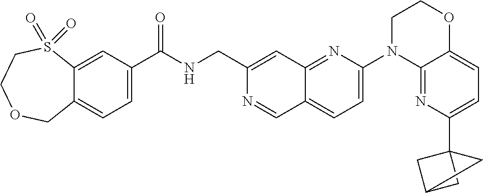 [SMILES: O=C(NCc1cc2nc(N3CCOc4ccc(C56CC(C5)C6)nc43)ccc2cn1)c1ccc2c(c1)S(=O)(=O)CCOC2] |
| 767 | 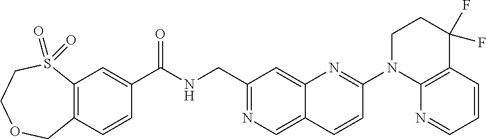 [SMILES: O=C(NCc1cc2nc(N3CCC(F)(F)c4cccnc43)ccc2cn1)c1ccc2c(c1)S(=O)(=O)CCOC2] |
| 768 | 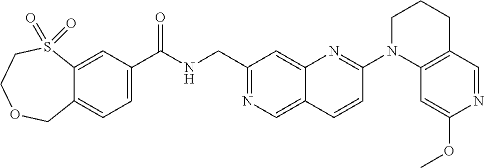 [SMILES: COc1cc2c(cn1)CCCN2c1ccc2cnc(CNC(=O)c3ccc4c(c3)S(=O)(=O)CCOC4)cc2n1] |
| 769 | 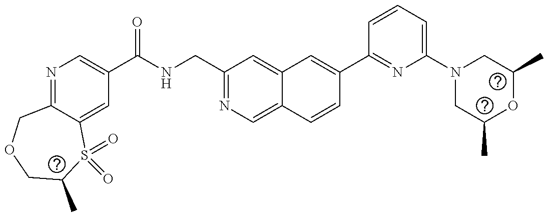 [SMILES: C[C@@H]1CN(c2cccc(-c3ccc4cnc(CNC(=O)c5cnc6c(c5)S(=O)(=O)[C@@H](C)COC6)cc4c3)n2)C[C@H](C)O1] |
| 770 | 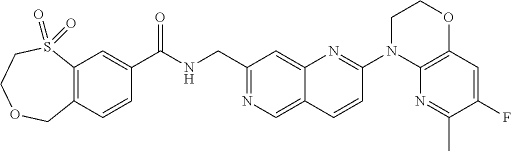 [SMILES: Cc1nc2c(cc1F)OCCN2c1ccc2cnc(CNC(=O)c3ccc4c(c3)S(=O)(=O)CCOC4)cc2n1] |
| 771 | 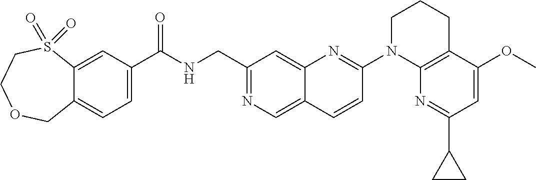 [SMILES: COc1cc(C2CC2)nc2c1CCCN2c1ccc2cnc(CNC(=O)c3ccc4c(c3)S(=O)(=O)CCOC4)cc2n1] |
| 772 | 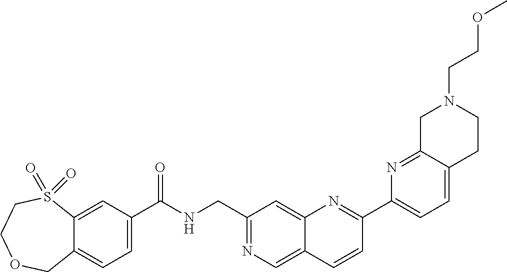 [SMILES: COCCN1CCc2ccc(-c3ccc4cnc(CNC(=O)c5ccc6c(c5)S(=O)(=O)CCOC6)cc4n3)nc2C1] |
| 773 | 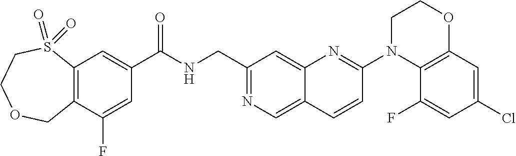 [SMILES: O=C(NCc1cc2nc(N3CCOc4cc(Cl)cc(F)c43)ccc2cn1)c1cc(F)c2c(c1)S(=O)(=O)CCOC2] |
| 774 | 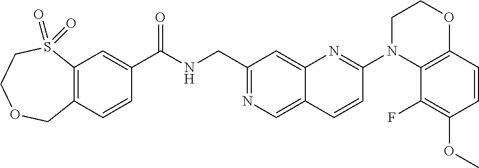 [SMILES: COc1ccc2c(c1F)N(c1ccc3cnc(CNC(=O)c4ccc5c(c4)S(=O)(=O)CCOC5)cc3n1)CCO2] |
| 775 | 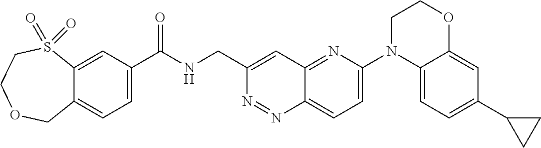 [SMILES: O=C(NCc1cc2nc(N3CCOc4cc(C5CC5)ccc43)ccc2nn1)c1ccc2c(c1)S(=O)(=O)CCOC2] |
| 776 | 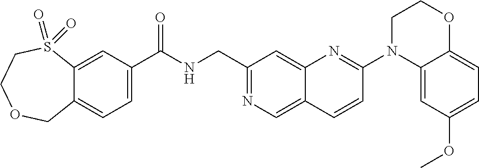 [SMILES: COc1ccc2c(c1)N(c1ccc3cnc(CNC(=O)c4ccc5c(c4)S(=O)(=O)CCOC5)cc3n1)CCO2] |
| 777 | 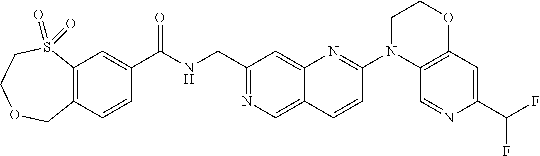 [SMILES: O=C(NCc1cc2nc(N3CCOc4cc(C(F)F)ncc43)ccc2cn1)c1ccc2c(c1)S(=O)(=O)CCOC2] |
| 778 | 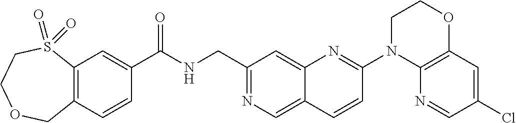 [SMILES: O=C(NCc1cc2nc(N3CCOc4cc(Cl)cnc43)ccc2cn1)c1ccc2c(c1)S(=O)(=O)CCOC2] |
| 779 | 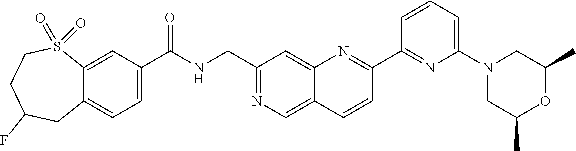 [SMILES: C[C@@H]1CN(c2cccc(-c3ccc4cnc(CNC(=O)c5ccc6c(c5)S(=O)(=O)CCC(F)C6)cc4n3)n2)C[C@H](C)O1] |
| 780 | 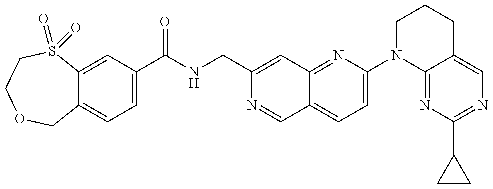 [SMILES: O=C(NCc1cc2nc(N3CCCc4cnc(C5CC5)nc43)ccc2cn1)c1ccc2c(c1)S(=O)(=O)CCOC2] |
| 781 | 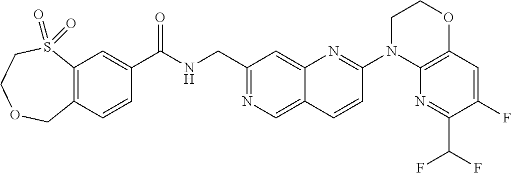 [SMILES: O=C(NCc1cc2nc(N3CCOc4cc(F)c(C(F)F)nc43)ccc2cn1)c1ccc2c(c1)S(=O)(=O)CCOC2] |
| 782 | 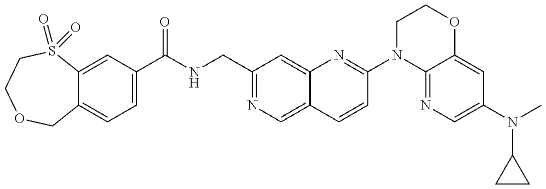 [SMILES: CN(c1cnc2c(c1)OCCN2c1ccc2cnc(CNC(=O)c3ccc4c(c3)S(=O)(=O)CCOC4)cc2n1)C1CC1] |
| 783 | 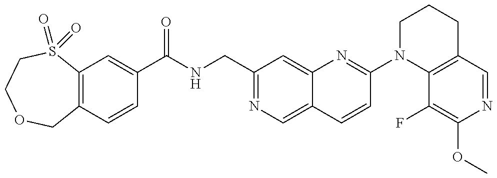 [SMILES: COc1ncc2c(c1F)N(c1ccc3cnc(CNC(=O)c4ccc5c(c4)S(=O)(=O)CCOC5)cc3n1)CCC2] |
| 784 | 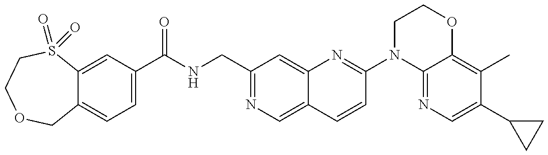 [SMILES: Cc1c(C2CC2)cnc2c1OCCN2c1ccc2cnc(CNC(=O)c3ccc4c(c3)S(=O)(=O)CCOC4)cc2n1] |
| 785 | 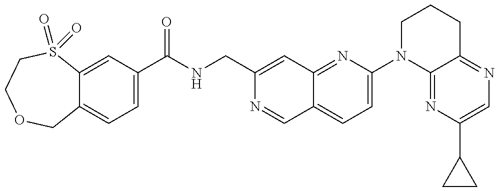 [SMILES: O=C(NCc1cc2nc(N3CCCc4ncc(C5CC5)nc43)ccc2cn1)c1ccc2c(c1)S(=O)(=O)CCOC2] |
| 786 | 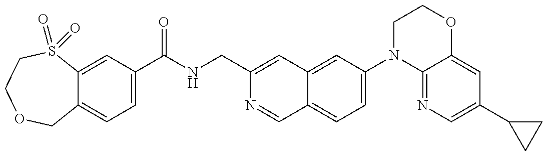 [SMILES: O=C(NCc1cc2cc(N3CCOc4cc(C5CC5)cnc43)ccc2cn1)c1ccc2c(c1)S(=O)(=O)CCOC2] |
| 787 | 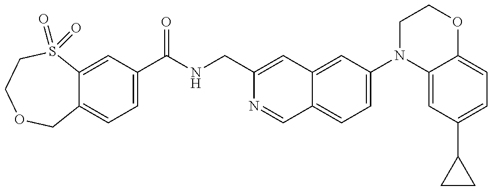 [SMILES: O=C(NCc1cc2cc(N3CCOc4ccc(C5CC5)cc43)ccc2cn1)c1ccc2c(c1)S(=O)(=O)CCOC2] |
| 788 | 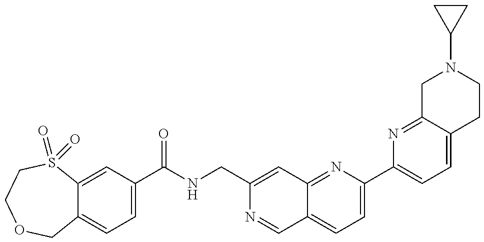 [SMILES: O=C(NCc1cc2nc(-c3ccc4c(n3)CN(C3CC3)CC4)ccc2cn1)c1ccc2c(c1)S(=O)(=O)CCOC2] |
| 789 | 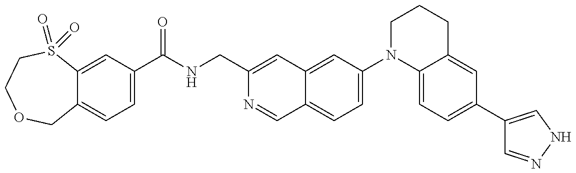 [SMILES: O=C(NCc1cc2cc(N3CCCc4cc(-c5cn[nH]c5)ccc43)ccc2cn1)c1ccc2c(c1)S(=O)(=O)CCOC2] |
| 790 | 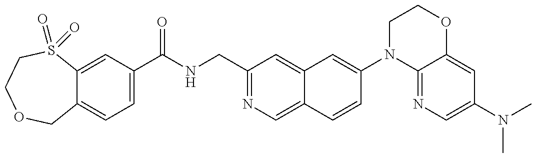 [SMILES: CN(C)c1cnc2c(c1)OCCN2c1ccc2cnc(CNC(=O)c3ccc4c(c3)S(=O)(=O)CCOC4)cc2c1] |
| 791 | 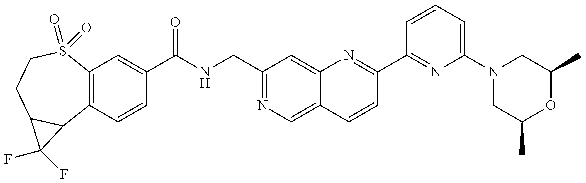 [SMILES: C[C@@H]1CN(c2cccc(-c3ccc4cnc(CNC(=O)c5ccc6c(c5)S(=O)(=O)CCC5C6C5(F)F)cc4n3)n2)C[C@H](C)O1] |
| 792 | 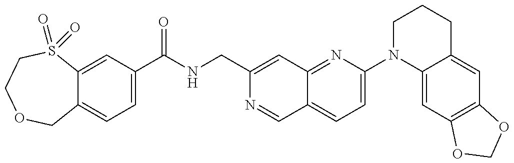 [SMILES: O=C(NCc1cc2nc(N3CCCc4cc5c(cc43)OCO5)ccc2cn1)c1ccc2c(c1)S(=O)(=O)CCOC2] |
| 793 | 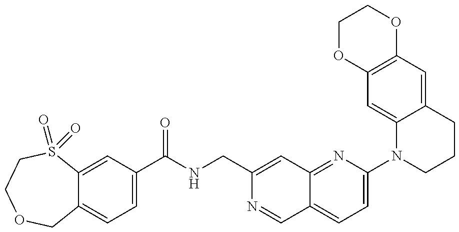 [SMILES: O=C(NCc1cc2nc(N3CCCc4cc5c(cc43)OCCO5)ccc2cn1)c1ccc2c(c1)S(=O)(=O)CCOC2] |
| 794 | 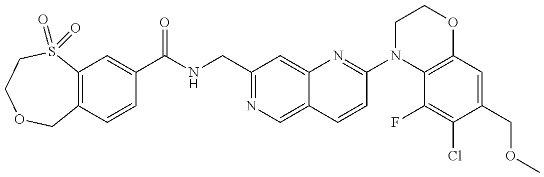 [SMILES: COCc1cc2c(c(F)c1Cl)N(c1ccc3cnc(CNC(=O)c4ccc5c(c4)S(=O)(=O)CCOC5)cc3n1)CCO2] |
| 795 | 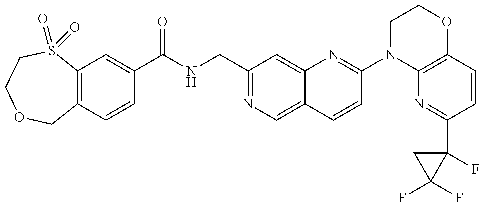 [SMILES: O=C(NCc1cc2nc(N3CCOc4ccc(C5(F)CC5(F)F)nc43)ccc2cn1)c1ccc2c(c1)S(=O)(=O)CCOC2] |
| 796 | 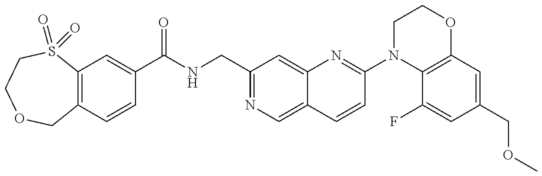 [SMILES: COCc1cc(F)c2c(c1)OCCN2c1ccc2cnc(CNC(=O)c3ccc4c(c3)S(=O)(=O)CCOC4)cc2n1] |
| 797 | 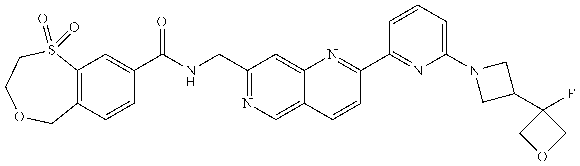 [SMILES: O=C(NCc1cc2nc(-c3cccc(N4CC(C5(F)COC5)C4)n3)ccc2cn1)c1ccc2c(c1)S(=O)(=O)CCOC2] |
| 798 | 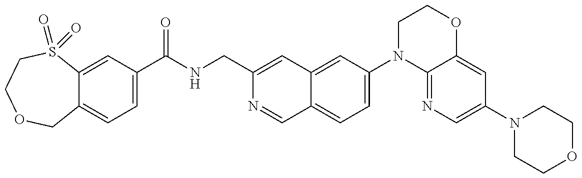 [SMILES: O=C(NCc1cc2cc(N3CCOc4cc(N5CCOCC5)cnc43)ccc2cn1)c1ccc2c(c1)S(=O)(=O)CCOC2] |
| 799 | 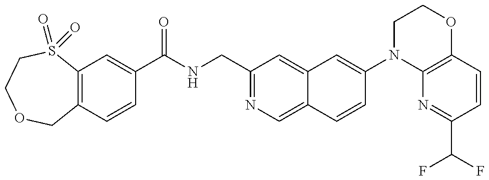 [SMILES: O=C(NCc1cc2cc(N3CCOc4ccc(C(F)F)nc43)ccc2cn1)c1ccc2c(c1)S(=O)(=O)CCOC2] |
| 800 | 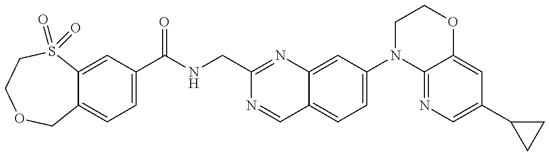 [SMILES: O=C(NCc1ncc2ccc(N3CCOc4cc(C5CC5)cnc43)cc2n1)c1ccc2c(c1)S(=O)(=O)CCOC2] |
| 801 | 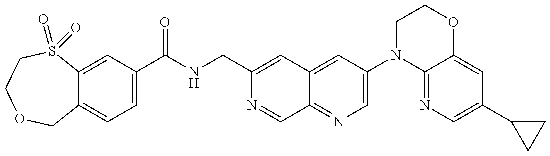 [SMILES: O=C(NCc1cc2cc(N3CCOc4cc(C5CC5)cnc43)cnc2cn1)c1ccc2c(c1)S(=O)(=O)CCOC2] |
| 802 | 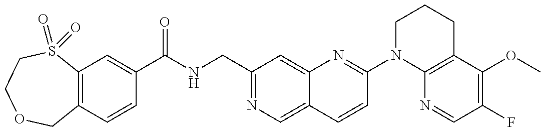 [SMILES: COc1c(F)cnc2c1CCCN2c1ccc2cnc(CNC(=O)c3ccc4c(c3)S(=O)(=O)CCOC4)cc2n1] |
| 803 | 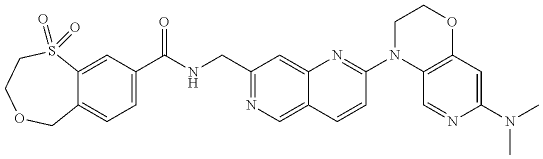 [SMILES: CN(C)c1cc2c(cn1)N(c1ccc3cnc(CNC(=O)c4ccc5c(c4)S(=O)(=O)CCOC5)cc3n1)CCO2] |
| 804 | 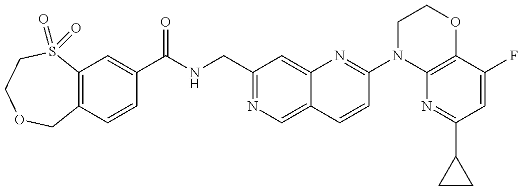 [SMILES: O=C(NCc1cc2nc(N3CCOc4c(F)cc(C5CC5)nc43)ccc2cn1)c1ccc2c(c1)S(=O)(=O)CCOC2] |
| 805 | 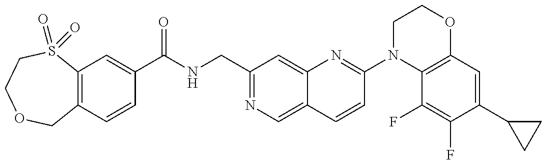 [SMILES: O=C(NCc1cc2nc(N3CCOc4cc(C5CC5)c(F)c(F)c43)ccc2cn1)c1ccc2c(c1)S(=O)(=O)CCOC2] |
| 806 | 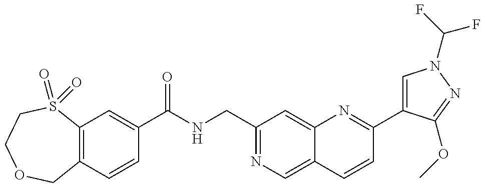 [SMILES: COc1nn(C(F)F)cc1-c1ccc2cnc(CNC(=O)c3ccc4c(c3)S(=O)(=O)CCOC4)cc2n1] |
| 807 | 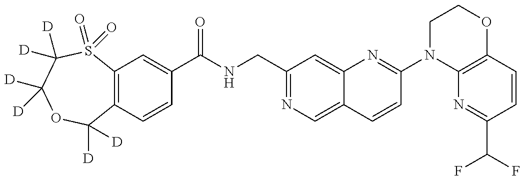 [SMILES: [2H]C1([2H])OC([2H])([2H])C([2H])([2H])S(=O)(=O)c2cc(C(=O)NCc3cc4nc(N5CCOc6ccc(C(F)F)nc65)ccc4cn3)ccc21] |
| 808 | 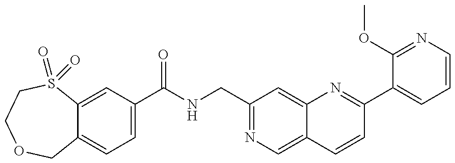 [SMILES: COc1ncccc1-c1ccc2cnc(CNC(=O)c3ccc4c(c3)S(=O)(=O)CCOC4)cc2n1] |
| 809 | 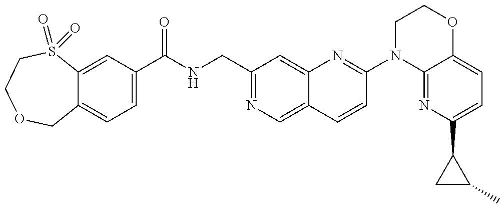 [SMILES: C[C@@H]1C[C@H]1c1ccc2c(n1)N(c1ccc3cnc(CNC(=O)c4ccc5c(c4)S(=O)(=O)CCOC5)cc3n1)CCO2] |
| 810 | 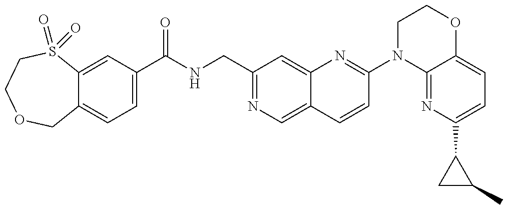 [SMILES: C[C@H]1C[C@@H]1c1ccc2c(n1)N(c1ccc3cnc(CNC(=O)c4ccc5c(c4)S(=O)(=O)CCOC5)cc3n1)CCO2] |
| 811 | 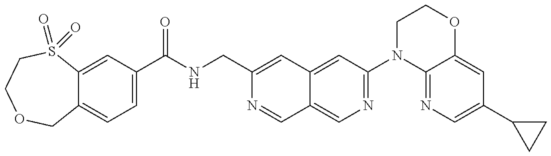 [SMILES: O=C(NCc1cc2cc(N3CCOc4cc(C5CC5)cnc43)ncc2cn1)c1ccc2c(c1)S(=O)(=O)CCOC2] |
| 812 | 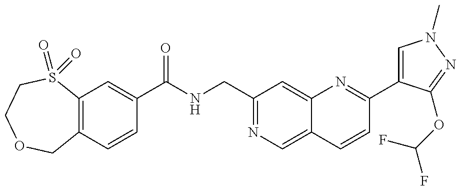 [SMILES: Cn1cc(-c2ccc3cnc(CNC(=O)c4ccc5c(c4)S(=O)(=O)CCOC5)cc3n2)c(OC(F)F)n1] |
| 813 | 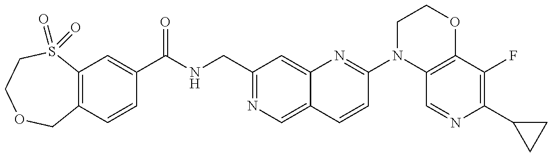 [SMILES: O=C(NCc1cc2nc(N3CCOc4c3cnc(C3CC3)c4F)ccc2cn1)c1ccc2c(c1)S(=O)(=O)CCOC2] |
| 814 | 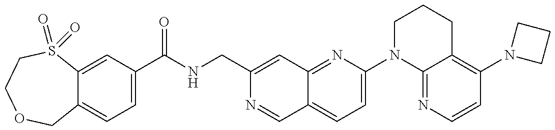 [SMILES: O=C(NCc1cc2nc(N3CCCc4c(N5CCC5)ccnc43)ccc2cn1)c1ccc2c(c1)S(=O)(=O)CCOC2] |
| 815 | 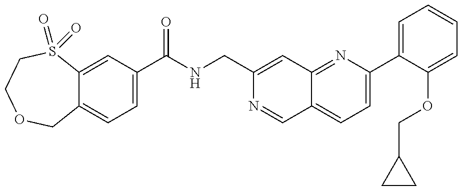 [SMILES: O=C(NCc1cc2nc(-c3ccccc3OCC3CC3)ccc2cn1)c1ccc2c(c1)S(=O)(=O)CCOC2] |
| 816 | 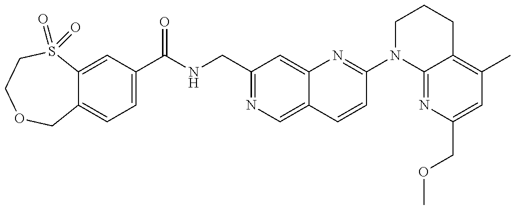 [SMILES: COCc1cc(C)c2c(n1)N(c1ccc3cnc(CNC(=O)c4ccc5c(c4)S(=O)(=O)CCOC5)cc3n1)CCC2] |
| 817 | 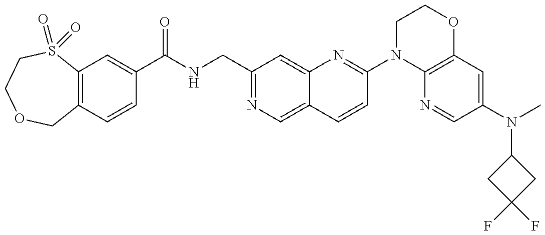 [SMILES: CN(c1cnc2c(c1)OCCN2c1ccc2cnc(CNC(=O)c3ccc4c(c3)S(=O)(=O)CCOC4)cc2n1)C1CC(F)(F)C1] |
| 818 | 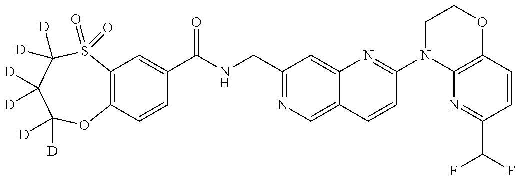 [SMILES: [2H]C1([2H])Oc2ccc(C(=O)NCc3cc4nc(N5CCOc6ccc(C(F)F)nc65)ccc4cn3)cc2S(=O)(=O)C([2H])([2H])C1([2H])[2H]] |
| 819 | 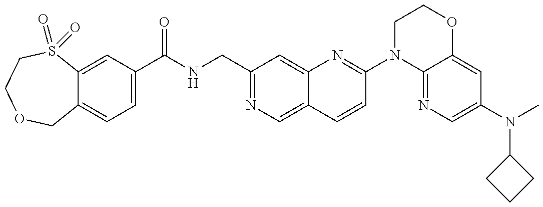 [SMILES: CN(c1cnc2c(c1)OCCN2c1ccc2cnc(CNC(=O)c3ccc4c(c3)S(=O)(=O)CCOC4)cc2n1)C1CCC1] |
| 820 | 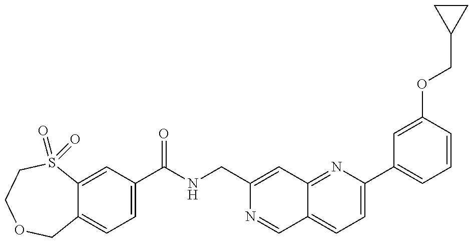 [SMILES: O=C(NCc1cc2nc(-c3cccc(OCC4CC4)c3)ccc2cn1)c1ccc2c(c1)S(=O)(=O)CCOC2] |
| 821 | 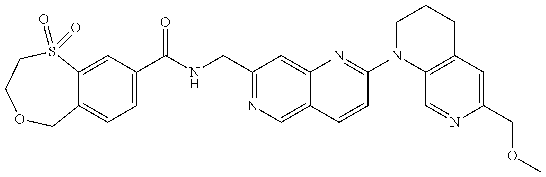 [SMILES: COCc1cc2c(cn1)N(c1ccc3cnc(CNC(=O)c4ccc5c(c4)S(=O)(=O)CCOC5)cc3n1)CCC2] |
| 822 | 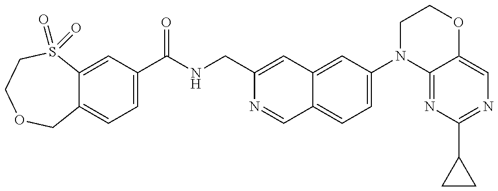 [SMILES: O=C(NCc1cc2cc(N3CCOc4cnc(C5CC5)nc43)ccc2cn1)c1ccc2c(c1)S(=O)(=O)CCOC2] |
| 823 | 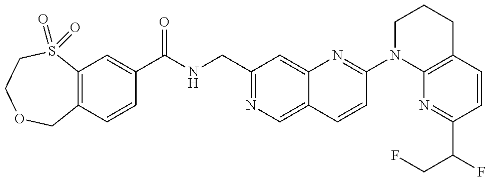 [SMILES: O=C(NCc1cc2nc(N3CCCc4ccc(C(F)CF)nc43)ccc2cn1)c1ccc2c(c1)S(=O)(=O)CCOC2] |
| 824 | 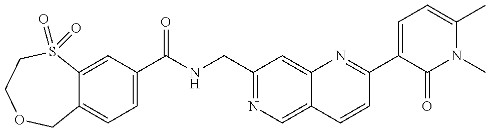 [SMILES: Cc1ccc(-c2ccc3cnc(CNC(=O)c4ccc5c(c4)S(=O)(=O)CCOC5)cc3n2)c(=O)n1C] |
| 825 | 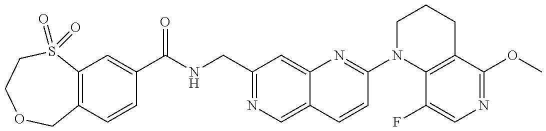 [SMILES: COc1ncc(F)c2c1CCCN2c1ccc2cnc(CNC(=O)c3ccc4c(c3)S(=O)(=O)CCOC4)cc2n1] |
| 826 | 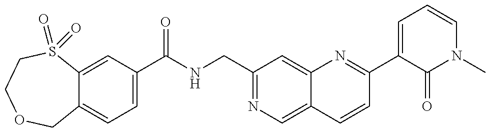 [SMILES: Cn1cccc(-c2ccc3cnc(CNC(=O)c4ccc5c(c4)S(=O)(=O)CCOC5)cc3n2)c1=O] |
| 827 | 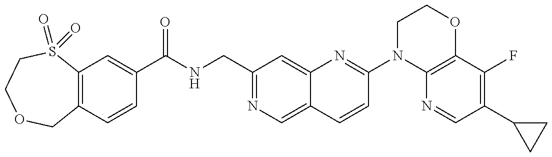 [SMILES: O=C(NCc1cc2nc(N3CCOc4c3ncc(C3CC3)c4F)ccc2cn1)c1ccc2c(c1)S(=O)(=O)CCOC2] |
| 828 | 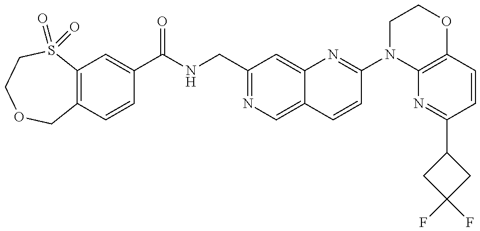 [SMILES: O=C(NCc1cc2nc(N3CCOc4ccc(C5CC(F)(F)C5)nc43)ccc2cn1)c1ccc2c(c1)S(=O)(=O)CCOC2] |
| 829 | 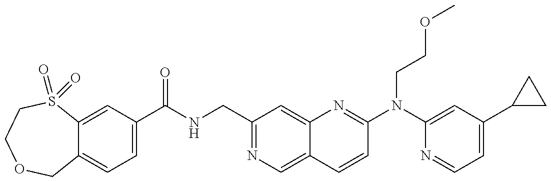 [SMILES: COCCN(c1cc(C2CC2)ccn1)c1ccc2cnc(CNC(=O)c3ccc4c(c3)S(=O)(=O)CCOC4)cc2n1] |
| 830 | 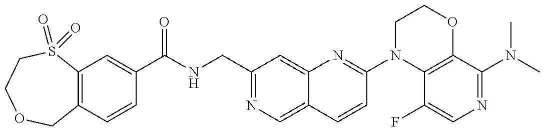 [SMILES: CN(C)c1ncc(F)c2c1OCCN2c1ccc2cnc(CNC(=O)c3ccc4c(c3)S(=O)(=O)CCOC4)cc2n1] |
| 831 | 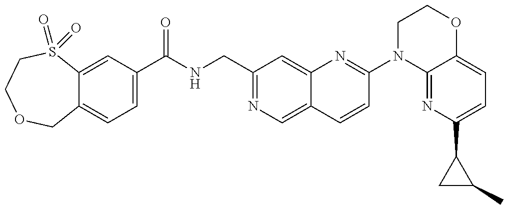 [SMILES: C[C@H]1C[C@H]1c1ccc2c(n1)N(c1ccc3cnc(CNC(=O)c4ccc5c(c4)S(=O)(=O)CCOC5)cc3n1)CCO2] |
| 832 | 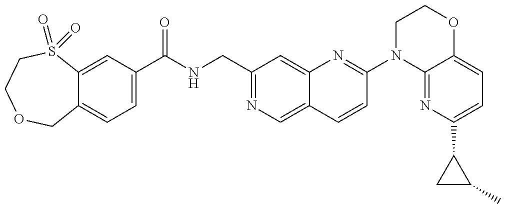 [SMILES: C[C@@H]1C[C@@H]1c1ccc2c(n1)N(c1ccc3cnc(CNC(=O)c4ccc5c(c4)S(=O)(=O)CCOC5)cc3n1)CCO2] |
| 833 | 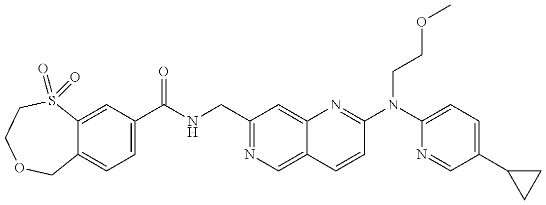 [SMILES: COCCN(c1ccc(C2CC2)cn1)c1ccc2cnc(CNC(=O)c3ccc4c(c3)S(=O)(=O)CCOC4)cc2n1] |
| 834 | 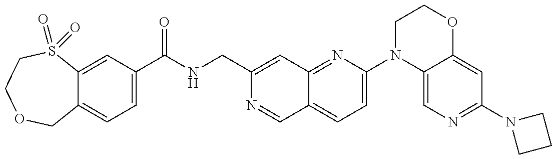 [SMILES: O=C(NCc1cc2nc(N3CCOc4cc(N5CCC5)ncc43)ccc2cn1)c1ccc2c(c1)S(=O)(=O)CCOC2] |
| 835 | 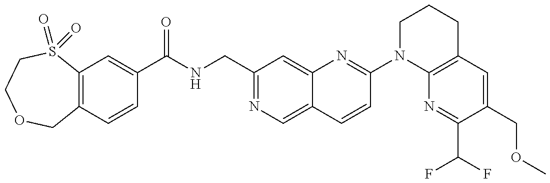 [SMILES: COCc1cc2c(nc1C(F)F)N(c1ccc3cnc(CNC(=O)c4ccc5c(c4)S(=O)(=O)CCOC5)cc3n1)CCC2] |
| 836 | 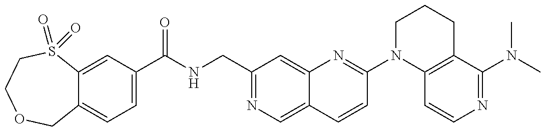 [SMILES: CN(C)c1nccc2c1CCCN2c1ccc2cnc(CNC(=O)c3ccc4c(c3)S(=O)(=O)CCOC4)cc2n1] |
| 837 | 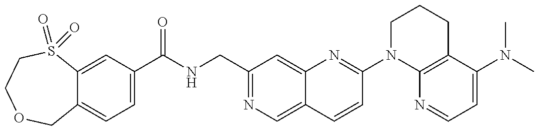 [SMILES: CN(C)c1ccnc2c1CCCN2c1ccc2cnc(CNC(=O)c3ccc4c(c3)S(=O)(=O)CCOC4)cc2n1] |
| 838 | 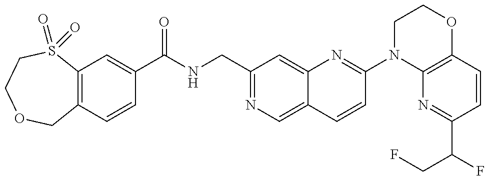 [SMILES: O=C(NCc1cc2nc(N3CCOc4ccc(C(F)CF)nc43)ccc2cn1)c1ccc2c(c1)S(=O)(=O)CCOC2] |
| 839 | 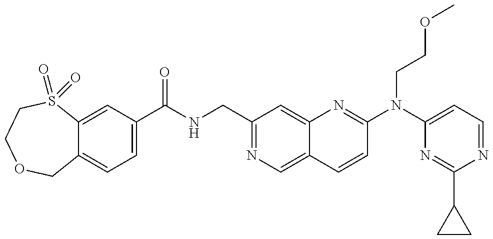 [SMILES: COCCN(c1ccnc(C2CC2)n1)c1ccc2cnc(CNC(=O)c3ccc4c(c3)S(=O)(=O)CCOC4)cc2n1] |
| 840 | 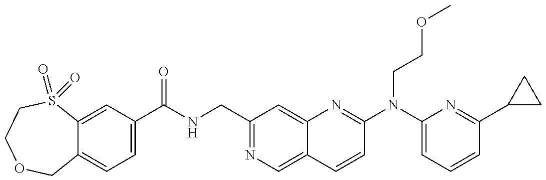 [SMILES: COCCN(c1cccc(C2CC2)n1)c1ccc2cnc(CNC(=O)c3ccc4c(c3)S(=O)(=O)CCOC4)cc2n1] |
| 841 | 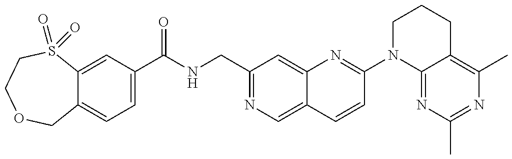 [SMILES: Cc1nc(C)c2c(n1)N(c1ccc3cnc(CNC(=O)c4ccc5c(c4)S(=O)(=O)CCOC5)cc3n1)CCC2] |
| 842 | 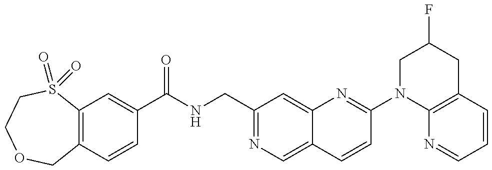 [SMILES: O=C(NCc1cc2nc(N3CC(F)Cc4cccnc43)ccc2cn1)c1ccc2c(c1)S(=O)(=O)CCOC2] |
| 843 | 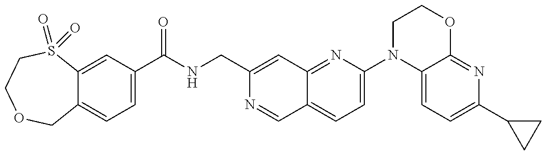 [SMILES: O=C(NCc1cc2nc(N3CCOc4nc(C5CC5)ccc43)ccc2cn1)c1ccc2c(c1)S(=O)(=O)CCOC2] |
| 844 | 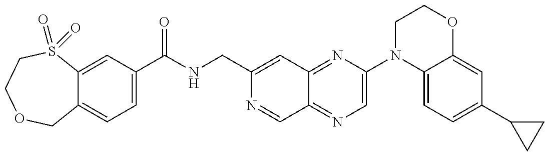 [SMILES: O=C(NCc1cc2nc(N3CCOc4cc(C5CC5)ccc43)cnc2cn1)c1ccc2c(c1)S(=O)(=O)CCOC2] |
| 845 | 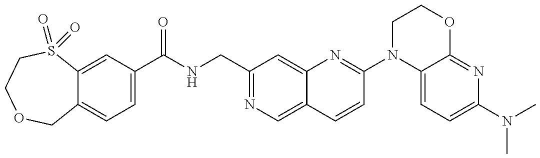 [SMILES: CN(C)c1ccc2c(n1)OCCN2c1ccc2cnc(CNC(=O)c3ccc4c(c3)S(=O)(=O)CCOC4)cc2n1] |
| 846 | 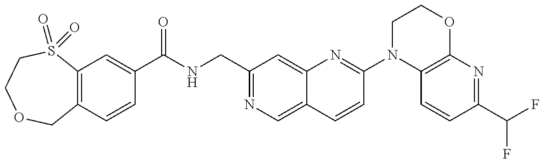 [SMILES: O=C(NCc1cc2nc(N3CCOc4nc(C(F)F)ccc43)ccc2cn1)c1ccc2c(c1)S(=O)(=O)CCOC2] |
| 847 | 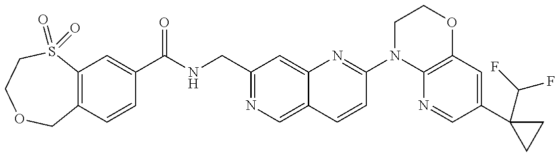 [SMILES: O=C(NCc1cc2nc(N3CCOc4cc(C5(C(F)F)CC5)cnc43)ccc2cn1)c1ccc2c(c1)S(=O)(=O)CCOC2] |
| 848 | 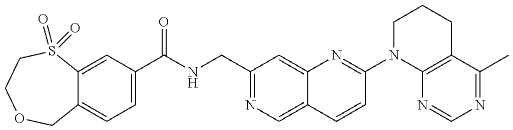 [SMILES: Cc1ncnc2c1CCCN2c1ccc2cnc(CNC(=O)c3ccc4c(c3)S(=O)(=O)CCOC4)cc2n1] |
| 849 | 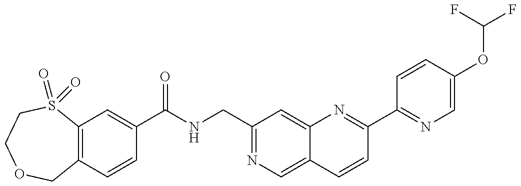 [SMILES: O=C(NCc1cc2nc(-c3ccc(OC(F)F)cn3)ccc2cn1)c1ccc2c(c1)S(=O)(=O)CCOC2] |
| 850 | 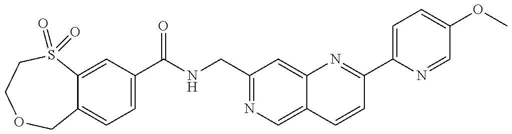 [SMILES: COc1ccc(-c2ccc3cnc(CNC(=O)c4ccc5c(c4)S(=O)(=O)CCOC5)cc3n2)nc1] |
| 851 | 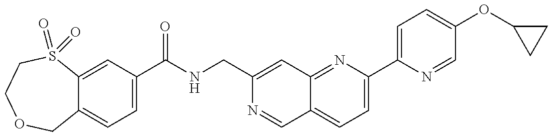 [SMILES: O=C(NCc1cc2nc(-c3ccc(OC4CC4)cn3)ccc2cn1)c1ccc2c(c1)S(=O)(=O)CCOC2] |
| 852 | 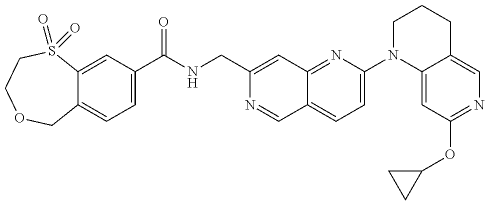 [SMILES: O=C(NCc1cc2nc(N3CCCc4cnc(OC5CC5)cc43)ccc2cn1)c1ccc2c(c1)S(=O)(=O)CCOC2] |
| 853 | 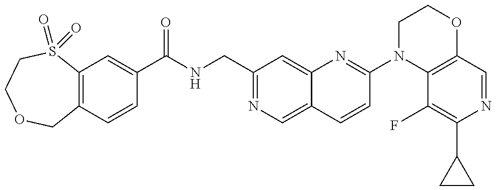 [SMILES: O=C(NCc1cc2nc(N3CCOc4cnc(C5CC5)c(F)c43)ccc2cn1)c1ccc2c(c1)S(=O)(=O)CCOC2] |
| 854 | 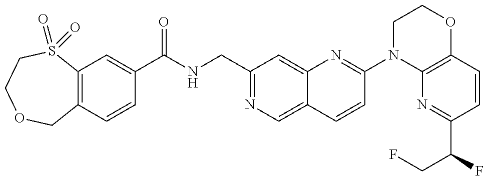 [SMILES: O=C(NCc1cc2nc(N3CCOc4ccc([C@@H](F)CF)nc43)ccc2cn1)c1ccc2c(c1)S(=O)(=O)CCOC2] |
| 855 | 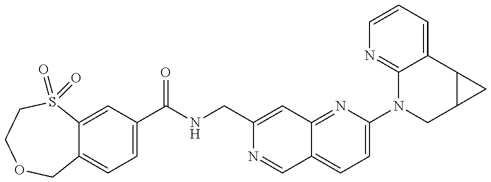 [SMILES: O=C(NCc1cc2nc(N3CC4CC4c4cccnc43)ccc2cn1)c1ccc2c(c1)S(=O)(=O)CCOC2] |
| 856 | 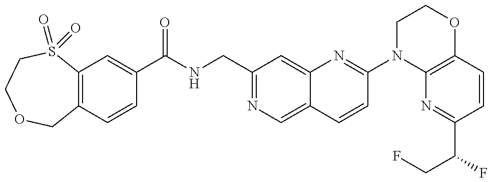 [SMILES: O=C(NCc1cc2nc(N3CCOc4ccc([C@H](F)CF)nc43)ccc2cn1)c1ccc2c(c1)S(=O)(=O)CCOC2] |

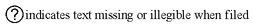

In some embodiments, the compound has a ratio of BRG1 IC₅₀ to BRM IC₅₀ of at least 5. In some embodiments, the compound has a ratio of BRG1 IC₅₀ to BRM IC₅₀ of at least 7. In some embodiments, the compound has a ratio of BRG1 IC₅₀ to BRM IC₅₀ of at least 10. In some embodiments, the compound has a ratio of BRG1 IC₅₀ to BRM IC₅₀ of at least 15. In some embodiments, the compound has a ratio of BRG1 IC₅₀ to BRM IC₅₀ of at least 20. In some embodiments, the compound has a ratio of BRG1 IC₅₀ to BRM IC₅₀ of at least 25. In some embodiments, the compound has a ratio of BRG1 IC₅₀ to BRM IC₅₀ of at least 30.

In another aspect, the invention features a pharmaceutical composition including any one of the above compounds and a pharmaceutically acceptable excipient.

In another aspect, the invention features a method of decreasing the activity of a BAF complex in a cell, the method involving contacting the cell with an effective amount of any of the foregoing compounds or a pharmaceutical composition thereof.

In some embodiments, the cell is a cancer cell.

In another aspect, the invention features a method of treating a BAF complex-related disorder in a subject in need thereof, the method involving administering to the subject an effective amount of any of the foregoing compounds or a pharmaceutical composition thereof.

In some embodiments, the BAF complex-related disorder is cancer.

In a further aspect, the invention features a method of inhibiting BRM, the method involving contacting a cell with an effective amount of any of the foregoing compounds or a pharmaceutical composition thereof. In some embodiments, the cell is a cancer cell.

In another aspect, the invention features a method of inhibiting BRG1, the method involving contacting the cell with an effective amount of any of the foregoing compounds or a pharmaceutical composition thereof. In some embodiments, the cell is a cancer cell.

In a further aspect, the invention features a method of inhibiting BRM and BRG1, the method involving contacting the cell with an effective amount of any of the foregoing compounds or a pharmaceutical composition thereof. In some embodiments, the cell is a cancer cell.

In another aspect, the invention features a method of treating a disorder related to a BRG1 loss of function mutation in a subject in need thereof, the method involving administering to the subject an effective amount of any of the foregoing compounds or a pharmaceutical composition thereof.

In some embodiments, the disorder related to a BRG1 loss of function mutation is cancer. In other embodiments, the subject is determined to have a BRG1 loss of function disorder, for example, is determined to have a BRG1 loss of function cancer (for example, the cancer has been determined to include cancer cells with loss of BRG1 function).

In another aspect, the invention features a method of inducing apoptosis in a cell, the method involving contacting the cell with an effective amount of any of the foregoing compounds or a pharmaceutical composition thereof. In some embodiments, the cell is a cancer cell.

In a further aspect, the invention features a method of treating cancer in a subject in need thereof, the method including administering to the subject an effective amount of any of the foregoing compounds or a pharmaceutical composition thereof.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobillary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, or penile cancer.

In some embodiments of any of the foregoing methods, the cancer is a drug resistant cancer or has failed to respond to a prior therapy (e.g., vemurafenib, dacarbazine, a CTLA4 inhibitor, a PD1 inhibitor, interferon therapy, a BRAF inhibitor, a MEK inhibitor, radiotherapy, temozolimide, irinotecan, a CAR-T therapy, herceptin, perjeta, tamoxifen, xeloda, docetaxol, platinum agents such as carboplatin, taxanes such as paclitaxel and docetaxel, ALK inhibitors, MET inihibitors, alimta, abraxane, Adriamycin®, gemcitabine, avaslin, halaven, neratinib, a PARP inhibitor, ARN810, an mTOR inhibitor, lopotecan, gemzar, a VEGFR2 inhibitor, a folate receptor antagonist, demcizumab, fosbretabulin, or a PDL1 inhibitor).

In some embodiments of any of the foregoing methods, the cancer has or has been determined to have BRG1 mutations. In some embodiments of any of the foregoing methods, the BRG1 mutations are homozygous. In some embodiments of any of the foregoing methods, the cancer does not have, or has been determined not to have, an epidermal growth factor receptor (EGFR) mutation. In some embodiments of any of the foregoing methods, the cancer does not have, or has been determined not to have, an anaplastic lymphoma kinase (ALK) driver mutation. In some embodiments of any of the foregoing methods, the cancer has, or has been determined to have, a KRAS mutation. In some embodiments of any of the foregoing methods, the BRG1 mutation is in the ATPase catalytic domain of the protein. In some embodiments of any of the foregoing methods, the BRG1 mutation is a deletion at the C-terminus of BRG1.

In another aspect, the disclosure provides a method treating a disorder related to BAF (e.g., cancer or viral infections) in a subject in need thereof. This method includes contacting a cell with an effective amount of any of the foregoing compounds, or pharmaceutically acceptable salts thereof, or any of the foregoing pharmaceutical compositions. In some embodiments, the disorder is a viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), Togaviridae family (e.g., Rubella virus). In some embodiments, the disorder is Coffin Siris, Neurofibromatosis (e.g., NF-1, NF-2, or Schwannomatosis), or Multiple Meningioma.

In another aspect, the disclosure provides a method for treating a viral infection in a subject in need thereof. This method includes administering to the subject an effective amount of any of the foregoing compounds, or pharmaceutically acceptable salts thereof, or any of the foregoing pharmaceutical compositions. In some embodiments, the viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV). Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), or Togaviridae family (e.g., Rubella virus).

In another aspect, the invention features a method of treating melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of reducing tumor growth of melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of suppressing metastatic progression of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of suppressing metastatic colonization of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of reducing the level and/or activity of BRG1 and/or BRM in a melanoma, prostate cancer, breast cancer, bone cancer, renal cell carcinoma, or hematologic cancer cell, the method including contacting the cell with an effective amount of any of the foregoing compounds or pharmaceutical compositions thereof.

In some embodiments of any of the above aspects, the melanoma, prostate cancer, breast cancer, bone cancer, renal cell carcinoma, or hematologic cell is in a subject.

In some embodiments of any of the above aspects, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRG1 by at least 50% (e.g., 55%, 60%, 65%, 70%, 75%, 80%, 85%, 90%, or 95%) as compared to a reference. In some embodiments, the effective amount of the compound that reduces the level and/or activity of BRG1 by at least 90% (e.g., 91%, 92%, 93%, 94%, 95%, 96%, 97%, 98%, or 99%).

In some embodiments, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRG1 by at least 5% (e.g., 6%, 7%, 8%, 9%, 10%, 15%, 20%, 25%, 30%, 35%, 40%, 45%, 50%, 55%, 80%, 65%, 70%, 75%, 80%, 85%, 90%, or 95%) as compared to a reference for at least 4 days (e.g., 5 days, 6 days, 7 days, 14 days, 28 days, or more).

In some embodiments of any of the above aspects, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 80%, 85%, 70%, 75%, 80%, 85%, 90%, or 95%) as compared to a reference. In some embodiments, the effective amount of the compound that reduces the level and/or activity of BRM by at least 50% (e.g., 55%, 60%, 65%, 70%, 75%, 80%, 85%, 90%, or 95%) as compared to a reference. In some embodiments, the effective amount of the compound that reduces the level and/or activity of BRM by at least 90% (e.g., 91%, 92%, 93%, 94%, 95%, 96%, 97%, 98%, or 99%).

In some embodiments, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 80%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRM by at least 5% (e.g., 6%, 7%, 8%, 9%, 10%, 15%, 20%, 25%, 30%, 35%, 40%, 45%, 50%, 55%, 80%, 65%, 70%, 75%, 80%, 85%, 90%, or 95%) as compared to a reference for at least 4 days (e.g., 5 days, 8 days, 7 days, 14 days, 28 days, or more).

In some embodiments, the subject has cancer. In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the cancer is melanoma (e.g., uveal melanoma, mucosal melanoma, or cutaneous melanoma). In some embodiments, the cancer is prostate cancer. In some embodiments, the cancer is a hematologic cancer, e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia (e.g., T-cell acute lymphoblastic leukemia or B-cell acute lymphoblastic leukemia), diffuse large cell lymphoma, or non-Hodgkin's lymphoma. In some embodiments, the cancer is breast cancer (e.g., an ER positive breast cancer, an ER negative breast cancer, triple positive breast cancer, or triple negative breast cancer). In some embodiments, the cancer is a bone cancer (e.g., Ewing's sarcoma). In some embodiments, the cancer is a renal cell carcinoma (e.g., a Microphthalmia Transcription Factor (MITF) family translocation renal cell carcinoma (tRCC)). In some embodiments, the cancer is metastatic (e.g., the cancer has spread to the liver). The metastatic cancer can include cells exhibiting migration and/or invasion of migrating cells and/or include cells exhibiting endothelial recruitment and/or angiogenesis. In other embodiments, the migrating cancer is a cell migration cancer. In still other embodiments, the cell migration cancer is a non-metastatic cell migration cancer. The metastatic cancer can be a cancer spread via seeding the surface of the peritoneal, pleural, pericardial, or subarachnoid spaces. Alternatively, the metastatic cancer can be a cancer spread via the lymphatic system, or a cancer spread hematogenously. In some embodiments, the effective amount of a compound of the invention is an amount effective to inhibit metastatic colonization of the cancer to the liver.

In some embodiments the cancer harbors a mutation in GNAQ. In some embodiments the cancer harbors a mutation in GNA11. In some embodiments the cancer harbors a mutation in PLCB4. In some embodiments the cancer harbors a mutation in CYSLTR2. In some embodiments the cancer harbors a mutation in BAP1. In some embodiments the cancer harbors a mutation in SF3B1. In some embodiments the cancer harbors a mutation in EIF1AX. In some embodiments the cancer harbors a TFE3 translocation. In some embodiments the cancer harbors a TFEB translocation. In some embodiments the cancer harbors a MITF translocation. In some embodiments the cancer harbors an EZH2 mutation. In some embodiments the cancer harbors a SUZ12 mutation. In some embodiments the cancer harbors an EED mutation.

In some embodiments of any of the foregoing methods, the method further includes administering to the subject or contacting the cell with an anticancer therapy, e.g., a chemotherapeutic or cytotoxic agent, immunotherapy, surgery, radiotherapy, thermotherapy, or photocoagulation, or a combination thereof. In some embodiments, the anticancer therapy is a chemotherapeutic or cytotoxic agent, e.g., an antimetabolite, antimitotic, antitumor antibiotic, asparagine-speclfic enzyme, bisphosphonates, antineoplastic, alkylating agent, DNA-Repair enzyme inhibitor, histone deacetylase inhibitor, corticosteroid, demethylating agent, immunomodulatory, Janus-associated kinase inhibitor, phosphinositide 3-kinase inhibitor, proteasome inhibitor, or tyrosine kinase inhibitor, or a combination thereof.

In some embodiments of any of the foregoing methods, the compound of the invention is used in combination with another anti-cancer therapy used for the treatment of uveal melanoma such as surgery, a MEK inhibitor, and/or a PKC inhibitor. For example, in some embodiments, the method further includes performing surgery prior to, subsequent to, or at the same time as administration of the compound of the invention. In some embodiments, the method further includes administration of a MEK inhibitor and/or a PKC inhibitor prior to, subsequent to, or at the same time as administration of the compound of the invention.

In some embodiments, the anticancer therapy and the compound of the invention are administered within 28 days of each other and each in an amount that together are effective to treat the subject.

In some embodiments, the subject or cancer has and/or has been identified as having a BRG1 loss of function mutation.

In some embodiments, the cancer is resistant to one or more chemotherapeutic or cytotoxic agents (e.g., the cancer has been determined to be resistant to chemotherapeutic or cytotoxic agents such as by genetic markers, or is likely to be resistant, to chemotherapeutic or cytotoxic agents such as a cancer that has failed to respond to a chemotherapeutic or cytotoxic agent). In some embodiments, the cancer has failed to respond to one or more chemotherapeutic or cytotoxic agents. In some embodiments, the cancer is resistant or has failed to respond to dacarbazine, temozolomide, cisplatin, treosulfan, fotemustine, IMCgp100, a CTLA-4 Inhibitor (e.g., ipilimumab), a PD-1 Inhibitor (e.g., Nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments, the cancer is resistant to or failed to respond to a previously administered therapeutic used for the treatment of uveal melanoma such as a MEK inhibitor or PKC inhibitor. For example, in some embodiments, the cancer is resistant to or failed to respond to a mitogen-activated protein kinase (MEK) inhibitor (e.g., selumetinib, binimetinib, or tametinib), and/or a protein kinase C (PKC) inhibitor (e.g., sotrastaurin or IDE196).

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a H or an alkyl group that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkenyl,” as used herein, refers to a branched or straight-chain monovalent saturated aliphatic hydrocarbon radical of 2 to 20 carbon atoms (e.g., 2 to 16 carbon atoms, 2 to 10 carbon atoms, or 2 to 6 carbon atoms). An alkenyl may be, e.g., monovalent or multivalent. One of skill in the art will recognize the number of applicable valencies from the context.

The term “alkyl.” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms). An alkyl may be, e.g., monovalent or multivalent. One of skill in the art will recognize the number of applicable valences from the context.

The term “amino,” as used herein, represents —N(R^{N1})₂, where each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), heteroaryl, or heterocyclyl, where each of these recited R^{N1} groups can be optionally substituted; or two R^{N1}, together with the atom to which they are attached, combine to form a heterocycyl or heteroaryl, and where each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl. An aryl may be, e.g., monovalent or multivalent. One of skill in the art will recognize the number of applicable valencies from the context.

The term “arylalkyl,” as used herein, represents an alkyl group substituted with an aryl group. Exemplary unsubstituted arylalkyl groups are from 7 to 30 carbons (e.g., from 7 to 18 or from 7 to 20 carbons, such as C₁-C₆ alkyl C₆-C₁₀ aryl, C₁-C₁₀ alkyl C₆-C₁₀ aryl, or C₁-C₂₀ oalkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl. A cycloalkyl may be, e.g., monovalent or multivalent. One of skill in the art will recognize the number of applicable valencies from the context.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkyl may be, e.g., monovalent or multivalent. One of skill in the art will recognize the number of applicable valencies from the context.

The term “heteroaryl,” as used herein, refers to a mono- or polycyclic radical of 5 to 14 (e.g., 5 to 12 or 5 to 10) atoms having at least one aromatic ring and containing 1, 2, or 3 ring atoms selected from nitrogen, oxygen, and sulfur, with the remaining ring atoms being carbon. In some embodiments, a heteroaryl is C₁-C₉ heteroaryl (e.g., C₂-C₆ heteroaryl). One or two ring carbon atoms of the heteroaryl group may be replaced with a carbonyl group. Examples of heteroaryl groups are pyridyl, pyrazolyl, benzooxazolyl, benzoimidazolyl, benzothiazolyl, imidazolyl, oxazolyl, thiazolyl, benzomorpholinyl, benzopiperidinyl, and indolinyl. A heteroaryl may be, e.g., monovalent or multivalent. One of skill in the art will recognize the number of applicable valencies from the context.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₂-C₉ heteroaryl C₁-C₆ alkyl, C₂-C₉ heteroaryl C₁-C₁₀ alkyl, or C₂-C₉ heteroaryl C₁-C₂₀ alkyl). In some embodiments, the alkyl and the heteroaryl each can be further substituted with 1, 2, 3, or 4 substituent groups as defined herein for the respective groups.

The term “heterocyclyl,” as used herein, refers a mono- or polycyclic radical having 3 to 14 (e.g., 4 to 12) atoms having at least one ring containing 1, 2, 3, or 4 ring atoms selected from N, O or S, where no ring is aromatic. In some embodiments, a heterocyclyl is a C₂-C₉ heterocyclyl. Examples of heterocyclyl groups include, but are not limited to, morpholinyl, thiomorpholinyl, piperazinyl, piperidinyl, pyranyl, pyrrolidinyl, tetrahydropyranyl, tetrahydrofuranyl, 1,3-dioxanyl, aza-oxybicyclo[4.3.0]nonyl, and aza-oxybicyclo[4.4.0]decyl. A heterocyclyl may be, e.g., monovalent or multivalent. One of skill in the art will recognize the number of applicable valencies from the context.

The term “heterocyclylalkyl,” as used herein, represents an alkyl group substituted with a heterocycyl group. Exemplary unsubstituted heterocycylalkyl groups are from 7 to 30 carbons (e.g., from 7 to 16 or from 7 to 20 carbons, such as C₂-C₉ heterocyclyl C₁-C₆ alkyl, C₂-C₆ heterocyclyl C₁-C₁₀ alkyl, or C₂-C₉ heterocyclyl C₁-C₂₀ alkyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chioroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenyithiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, heteroalkyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, where such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer.

Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” Indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HBRM-associated factors complex in a human cell.

As used herein, the term “BAF complex-related disorder” refers to a disorder that is caused or affected by the level of activity of a BAF complex.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By “determining the level” of a protein or RNA is meant the detection of a protein or an RNA, by methods known in the art, either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third party laboratory that directly acquired the physical entity or value). Methods to measure protein level generally include, but are not limited to, western blotting, immunoblotting, enzyme-linked immunosorbent assay (ELISA), radloimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels are known in the art and include, but are not limited to, quantitative polymerase chain reaction (qPCR) and Northern blot analyses.

By a “decreased level” or an “increased level” of a protein or RNA is meant a decrease or increase, respectively, in a protein or RNA level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20 fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein in a sample.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

As used herein, the term “Inhibiting BRM” refers to blocking or reducing the level or activity of the ATPase catalytic binding domain or the bromodomain of the protein. BRM inhibition may be determined using methods known in the art, e.g., a BRM ATPase assay, a Nano DSF assay, or a BRM Luciferase cell assay.

As used herein, the term “LXS196,” also known as IDE196, refers to the PKC inhibitor having the structure:

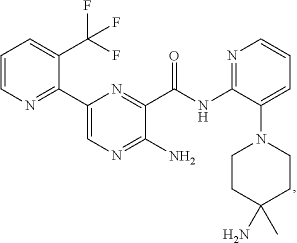 [SMILES: CC1(N)CCN(c2cccnc2NC(=O)c2nc(-c3ncccc3C(F)(F)F)cnc2N)CC1]

or a pharmaceutically acceptable salt thereof.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

As used herein, the term “pharmaceutically acceptable salt” means any pharmaceutically acceptable salt of a compound, for example, any compound of Formula I. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 68:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a ‘normal control’ or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the invention; a sample from a subject that has been treated by a compound of the invention; or a sample of a purified protein or RNA (e.g., any described herein) at a known normal concentration. By “reference standard or level” Is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the invention. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. Compounds of the invention may also be used to “prophylactically treat” or “prevent” a disorder, for example, in a subject at increased risk of developing the disorder.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a graph illustrating inhibition of cell proliferation of several cancer cell lines by a BRG1/BRM inhibitor (Compound A).

FIG. 2A is a graph illustrating inhibition of cell proliferation of uveal melanoma cell line 92-1 by a BRG1/BRM inhibitor (Compound A), a MEK inhibitor (Selumetinib), and a PKC inhibitor (LXS196).

FIG. 2B is a graph illustrating inhibition of cell proliferation of uveal melanoma cell line MP41 by a BRG1/BRM inhibitor (Compound A), a MEK inhibitor (Selumetinib), and a PKC inhibitor (LXS196).

FIG. 3 is a graph illustrating inhibition of cell proliferation of several cancer cell lines by a BRG1/BRM inhibitor (Compound B).

FIG. 4 is a graph illustrating the area under the curves (AUCs) calculated from dose-response curves for cancer cell lines treated with a BRG1/BRM inhibitor.

FIG. 5 is a graph illustrating inhibition of cell proliferation of uveal melanoma and non-small cell lung cancer cell lines by a BRG1/BRM inhibitor (Compound B).

FIG. 6A is a graph illustrating inhibition of cell proliferation of uveal melanoma cell line 92-1 by a BRG1/BRM inhibitor (Compound B), a MEK inhibitor (Selumetinib), and a PKC inhibitor (LXS196).

FIG. 6B is a graph illustrating inhibition of cell proliferation of uveal melanoma cell line MP41 by a BRG1/BRM inhibitor (Compound B), a MEK inhibitor (Selumetinib), and a PKC inhibitor (LXS196).

FIG. 7A is a graph illustrating inhibition of cell proliferation of parental and PKC-inhibitor refractory uveal melanoma cell lines by a PKC inhibitor (LXS196).

FIG. 7B is a graph illustrating inhibition of cell proliferation of parental and PKC-inhibitor refractory uveal melanoma cell lines by a BRG1/BRM inhibitor (Compound B).

FIG. 8A is a graph illustrating inhibition of tumor growth in mice engrafted with uveal melanoma cell lines by a BRG1/BRM inhibitor (Compound C).

FIG. 8B is an illustration of the size of tumors from mice engrafted with uveal melanoma cell lines and dosed with a BRG1/BRM inhibitor (Compound C).

FIG. 8C is a graph illustrating body weight change of mice engrafted with uveal melanoma cell lines and dosed with a BRG1/BRM inhibitor (Compound C).

### DETAILED DESCRIPTION

The present disclosure features compounds useful for the inhibition of BRM and optionally BRG1. These compounds may be used to modulate the activity of a BAF complex, for example, for the treatment of a BAF-related disorder, such as cancer (e.g., BRG1-loss of function disorders). Exemplary compounds described herein include compounds having a structure according to Formula I:

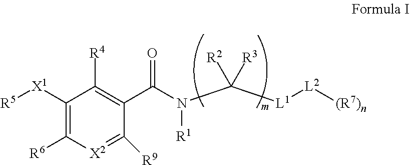

where
m is 0, 1, 2, or 3;
n is 0, 1, 2, 3, or 4;
X¹ is —S—, —SO—, —SO₂—, or —S(O)(NH)—;
X² is N or CR⁸;
R¹ is hydrogen or optionally substituted C₁-C₆ alkyl;
each R² and each R³ are independently hydrogen, optionally substituted C₁-C₆ alkyl, or optionally substituted C₁-C₆ heteroalkyl;
L¹ is optionally substituted 9- or 10-membered bicyclic heterocyclyl or optionally substituted 9- or 10-membered bicyclic heteroaryl;
L² is absent, optionally substituted C₃-C₁₀ cycloalkyl, optionally substituted C₆-C₁₀ aryl, optionally substituted 5- to 14-membered heteroaryl, or optionally substituted 4- to 14-membered heterocyclyl;
R⁴ is hydrogen, halo, optionally substituted C₁-C₆ alkyl, or optionally substituted C₃-C₁₀ cycloalkyl;
R⁵ is optionally substituted C₁-C₁₀ alkyl, optionally substituted C₁-C₁₀ heteroalkyl, or optionally substituted amino, and R⁶ is hydrogen, halo, cyano, optionally substituted C₁-C₆ alkyl, optionally substituted C₂-C₆ alkenyl, or optionally substituted C₃-C₁₀ cycloalkyl; or R⁵ and R⁶, together with the atoms to which they are attached, combine to form an optionally substituted 5- to 8-membered heterocyclyl;
each R⁷ is independently optionally substituted C₁-C₆ alkyl, optionally substituted C₁-C₆ heteroalkyl, halo, optionally substituted C₃-C₁₀ cycloalkyl, optionally substituted C₃-C₁₀ cycloalkyl C₁-C₆ alkyl, optionally substituted 5- to 14-membered heteroaryl, optionally substituted 4- to 14-membered heterocyclyl, —N(R^{7A})₂, or —OR^{7A}, wherein each R^{7A} is independently H, optionally substituted C₁-C₆ alkyl, optionally substituted C₁-C₈ heteroalkyl, optionally substituted C₃-C₁₀ cycloalkyl, optionally substituted C₆-C₁₀ aryl, optionally substituted 5- to 10-membered heteroaryl, or optionally substituted 4- to 10-membered heterocyclyl, or two geminal R^{7A} groups, together with the atom to which they are attached, combine to form optionally substituted 5- to 10-membered heteroaryl or optionally substituted 4- to 10-membered heterocyclyl; or two geminal R⁷ groups, together, with the atom to which they are attached, combine to form carbonyl;
R⁸ is hydrogen, halo, optionally substituted C₁-C₆ alkyl, or optionally substituted C₃-C₁₀ cycloalkyl; and
R⁹ is hydrogen or halo;
or a pharmaceutically acceptable salt thereof.

In some embodiments, the compound, or pharmaceutically acceptable salt thereof, has the structure of any one of compounds 1-308 in Table 1A. In some embodiments, the compound, or pharmaceutically acceptable salt thereof, has the structure of any one of compounds 309-856 in Table 1B.

Other embodiments, as well as exemplary methods for the synthesis of production of these compounds, are described herein.

### Pharmaceutical Uses

The compounds described herein are useful in the methods of the invention and, while not bound by theory, are believed to exert their ability to modulate the level, status, and/or activity of a BAF complex, I.e., by inhibiting the activity of the BRG1 and/or BRM proteins within the BAF complex in a mammal. BAF complex-related disorders include, but are not limited to, BRG1 loss of function mutation-related disorders.

An aspect of the present invention relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the compound is administered in an amount and for a time effective to result in one or more (e.g., two or more, three or more, four or more) of: (a) reduced tumor size, (b) reduced rate of tumor growth, (c) increased tumor cell death (d) reduced tumor progression, (e) reduced number of metastases, (t) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 80%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobillary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer and penile cancer.

### Combination Formulations and Uses Thereof

The compounds of the invention can be combined with one or more therapeutic agents. In particular, the therapeutic agent can be one that treats or prophylactically treats any cancer described herein.

### Combination Therapies

A compound of the invention can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethlylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (Including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enedlyne antibiotics (e.g., calicheamicin, especially calicheamicin gammall and calicheamicin omega (see, e.g., Agnew. Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), adaclnomysins, actinomycin, authramycln, azaserine, bleomycins, cactinomycln, carabicin, caminomycin, carzinophilin, chromomycinis, dactinomycin, daunorubicin, detorubicin, 6-diazo-5-oxo-L-norieucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatrexate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T 2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paditaxel (Bristol-Myers Squibb Oncology, Princeton, N.J.), ABraxane®, cremophor-free, albumin-engineered nanoparticle formulation of paditaxel (American Pharmaceutical Partners, Schaumberg, Ill.), and Taxotere® doxetaxel (Rhone-Poulenc Rorer, Antony, France); chioranbucll; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include Rituxan (Rituximab); Zenapax (Daulizumab); Simulect (Basiliximab); Synagis (Palivizumab); Remicade (Infliximab); Herceptin (Trastuzumab); Mylotarg (Gemtuzumab ozogamicin); Campath (Alemtuzumab); Zevalin (Ibritumomab tiuxetan); Humira (Adalimumab); Xolair (Omalizumab); Bexxar (Tositumomab-I-131); Raptiva (Efalizumab); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Harts (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); and Gazyva (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 938559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD180, CGEN-15049, CHK1, CHK2, A2aR, B-7 family ligands, or a combination thereof.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The compounds of the invention are preferably formulated into pharmaceutical compositions for administration to a mammal, preferably, a human, in a biologically compatible form suitable for administration in vivo. Accordingly, in an aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or It may be enclosed in hard- or soft-shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers. A compound of the invention may also be administered parenterally. Solutions of a compound of the invention can be prepared in water suitably mixed with a surfactant. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20th ed.) and in The United States Pharmacopeia: The National Formulary (USP 24 NF19), published in 1999. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant. The aerosol dosage forms can also take the form of a pump-atomizer. Compositions suitable for buccal or sublingual administration include tablets, lozenges, and pastilles, where the active ingredient is formulated with a carrier. Compositions for rectal administration are conveniently in the form of suppositories containing a conventional suppository base. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg. Dose ranges include, for example, between 10-1000 mg.

Alternatively, the dosage amount can be calculated using the body weight of the patient. For example, the dose of a compound, or pharmaceutical composition thereof, administered to a patient may range from 0.1-100 mg/kg.

### EXAMPLES

Definitions used in the following Schemes and elsewhere herein are:
MeCN or ACN acetonitrile
AIBN azobisisobutyronitrile
Boc tert-butoxycarbonyl
t-BuOK potassium tert-butoxide
DAST diethylaminosulfur trifluoride
DCE dichioroethane
DCM dichioromethane
DCPP-2HBF₄ 1,3-bis(dicyclohexylphosphino)propane bis(tetrafluoroborate)
DEA N,N-diethylamine
DMP Dess-Martin periodinane, 1,1,1-Tris(acetyloxy)-1,1-dihydro-1,2-
DIAD diisopropyl azodicarboxylate
DIBAL-H dilsobutylaluminum hydride
DIEA or DIPEA N,N-diisopropylethylamine
DMA dimethylacetamide
DMAP 4-(dimethylamino)pyridine
DME 1,2-dimethoxyethane
DMF N,N-dimethylformamide
DMSO dimethylsulfoxide
dppf bis(diphenylphosphino)ferrocene
EDCl 1-ethyl-(3-dimethylaminopropyl)carbodimide hydrochloride
ESI electrospray ionization
Et₃N or TEA triethylamine
EA ethyl acetate
EtOH ethyl alcohol
FA formic acid
FCC flash column chromatography
g grams
HATU 2-(3H-[1,2,3]triazolo[4,5-b]pyridin-3-yl)-1,3,3-tetramethylisouronium
HCl hydrochloric acid
HOAc acetic acid
HOBt hydroxybenzotriazole
HPLC high performance liquid chromatography
IPA isopropyl alcohol
L liter
LCMS liquid chromatography/mass spectrometry
m-CPBA 3-chloroperoxybenzoic acid
MeCN acetonitrile
MeI methyl iodide
MeOH methyl alcohol
mL milliliter
mmol millimole
mg milligrams
MHz megahertz
MS mass spectrometry
MTBE methyl tart-butyl ether
m/z mass/charge ratio
NBS N-bromosuccinimide
NIS N-iodosuccinimide
nm nanometer
NMR nuclear magnetic resonance
PE petroleum ether
PhMe toluene
ppm parts per million
rt room temperature
RT retention time
SFC supercritical fluid chromatography
SPhos Pd G3 (2-dicyclohexylphosphino-2′,6′-dimethoxybiphenyl) [2-(2′-amino-1,1′-biphenyl)]palladium(I I) methanesulfonate
TBS tert-butyldimethylsilyl
TBSCl tert-butyldimethylsilyl chloride
TBDMS tert-butyldimethylsilyl chloride
TFA trifluoroacetic acid
TFAA trifluoroacetic anhydride
THE tetrahydrofuran
TMSCN trimethylsilyl cyanide
TosMIC toluenesulfonylmethyl isocyanide
Ziram zinc dimethyldithiocarbamate

### Materials

Unless otherwise noted, all materials were obtained from commercial suppliers and were used without further purification. All reactions involving air- or moisture-sensitive reagents were performed under a nitrogen atmosphere.

Table 1C lists compounds of the invention prepared using methods described herein.

TABLE 1C
Compounds of the invention

| # | Compound |
|---|---|
| 304 | 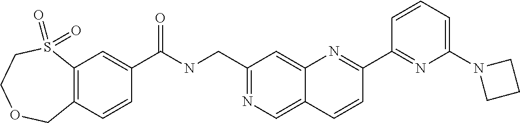 [SMILES: O=C(NCc1cc2nc(-c3cccc(N4CCC4)n3)ccc2cn1)c1ccc2c(c1)S(=O)(=O)CCOC2] |
| 184 | 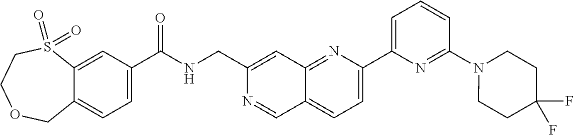 [SMILES: O=C(NCc1cc2nc(-c3cccc(N4CCC(F)(F)CC4)n3)ccc2cn1)c1ccc2c(c1)S(=O)(=O)CCOC2] |
| 303 | 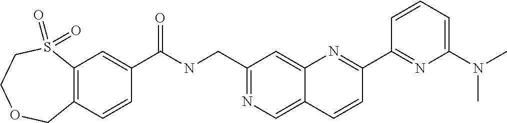 [SMILES: CN(C)c1cccc(-c2ccc3cnc(CNC(=O)c4ccc5c(c4)S(=O)(=O)CCOC5)cc3n2)n1] |
| 181 | 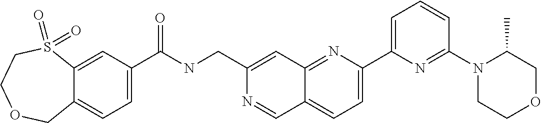 [SMILES: C[C@@H]1COCCN1c1cccc(-c2ccc3cnc(CNC(=O)c4ccc5c(c4)S(=O)(=O)CCOC5)cc3n2)n1] |
| 155 | 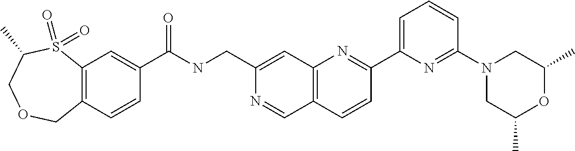 |
| 178 | 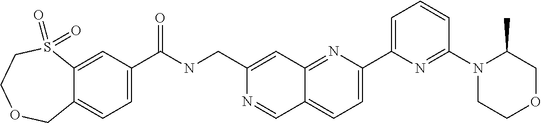 [SMILES: C[C@H]1COCCN1c1cccc(-c2ccc3cnc(CNC(=O)c4ccc5c(c4)S(=O)(=O)CCOC5)cc3n2)n1] |
| 159 | 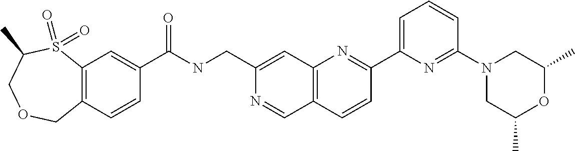 |
| 174 | 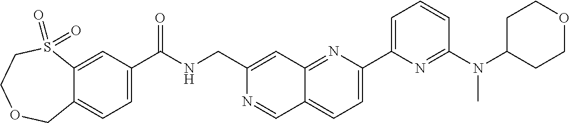 [SMILES: CN(c1cccc(-c2ccc3cnc(CNC(=O)c4ccc5c(c4)S(=O)(=O)CCOC5)cc3n2)n1)C1CCOCC1] |
| 282 | 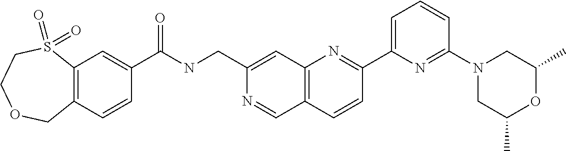 [SMILES: C[C@@H]1CN(c2cccc(-c3ccc4cnc(CNC(=O)c5ccc6c(c5)S(=O)(=O)CCOC6)cc4n3)n2)C[C@H](C)O1] |
| 145 | 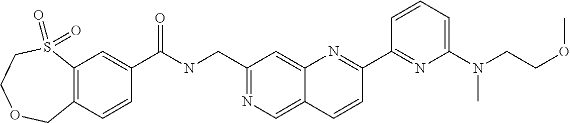 [SMILES: COCCN(C)c1cccc(-c2ccc3cnc(CNC(=O)c4ccc5c(c4)S(=O)(=O)CCOC5)cc3n2)n1] |
| 190 | 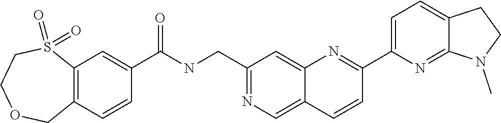 [SMILES: CN1CCc2ccc(-c3ccc4cnc(CNC(=O)c5ccc6c(c5)S(=O)(=O)CCOC6)cc4n3)nc21] |
| 144 | 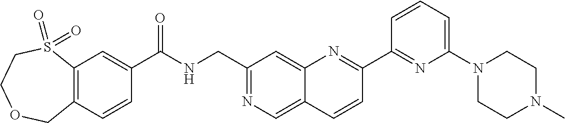 [SMILES: CN1CCN(c2cccc(-c3ccc4cnc(CNC(=O)c5ccc6c(c5)S(=O)(=O)CCOC6)cc4n3)n2)CC1] |
| 176 | 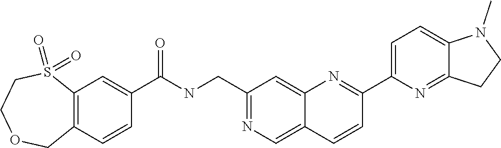 [SMILES: CN1CCc2nc(-c3ccc4cnc(CNC(=O)c5ccc6c(c5)S(=O)(=O)CCOC6)cc4n3)ccc21] |
| 228 | 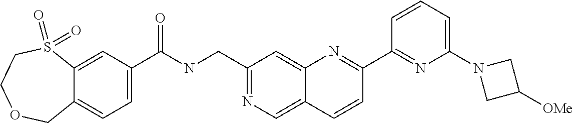 [SMILES: COC1CN(c2cccc(-c3ccc4cnc(CNC(=O)c5ccc6c(c5)S(=O)(=O)CCOC6)cc4n3)n2)C1] |
| 167 | 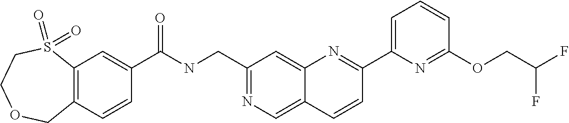 [SMILES: O=C(NCc1cc2nc(-c3cccc(OCC(F)F)n3)ccc2cn1)c1ccc2c(c1)S(=O)(=O)CCOC2] |
| 226 | 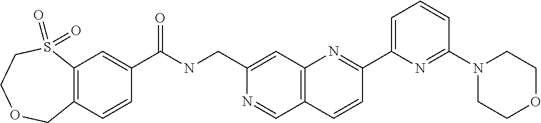 [SMILES: O=C(NCc1cc2nc(-c3cccc(N4CCOCC4)n3)ccc2cn1)c1ccc2c(c1)S(=O)(=O)CCOC2] |
| 165 | 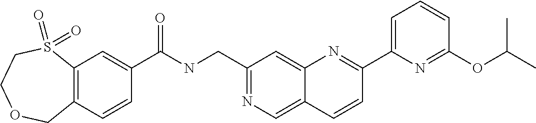 [SMILES: CC(C)Oc1cccc(-c2ccc3cnc(CNC(=O)c4ccc5c(c4)S(=O)(=O)CCOC5)cc3n2)n1] |
| 227 | 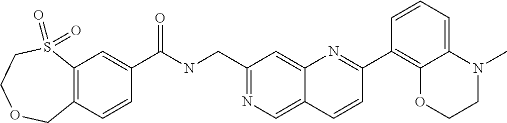 [SMILES: CN1CCOc2c(-c3ccc4cnc(CNC(=O)c5ccc6c(c5)S(=O)(=O)CCOC6)cc4n3)cccc21] |
| 240 | 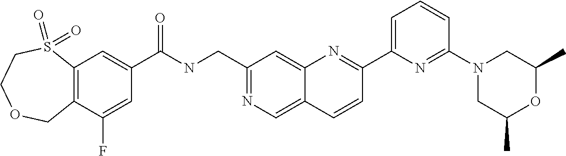 [SMILES: C[C@@H]1CN(c2cccc(-c3ccc4cnc(CNC(=O)c5cc(F)c6c(c5)S(=O)(=O)CCOC6)cc4n3)n2)C[C@H](C)O1] |
| 307 | 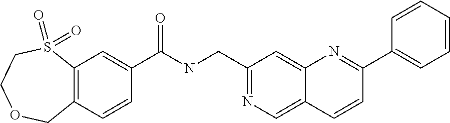 [SMILES: O=C(NCc1cc2nc(-c3ccccc3)ccc2cn1)c1ccc2c(c1)S(=O)(=O)CCOC2] |
| 143 | 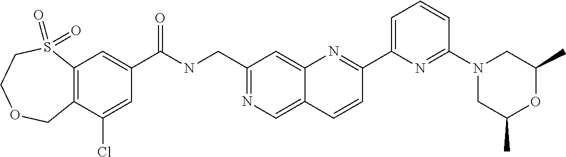 [SMILES: C[C@@H]1CN(c2cccc(-c3ccc4cnc(CNC(=O)c5cc(Cl)c6c(c5)S(=O)(=O)CCOC6)cc4n3)n2)C[C@H](C)O1] |
| 196 | 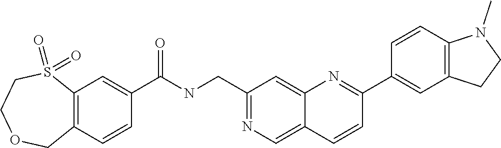 [SMILES: CN1CCc2cc(-c3ccc4cnc(CNC(=O)c5ccc6c(c5)S(=O)(=O)CCOC6)cc4n3)ccc21] |
| 284 | 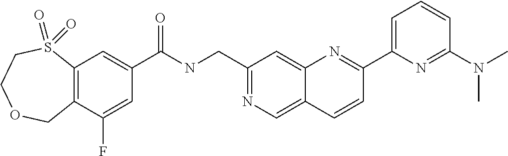 [SMILES: CN(C)c1cccc(-c2ccc3cnc(CNC(=O)c4cc(F)c5c(c4)S(=O)(=O)CCOC5)cc3n2)n1] |
| 193 | 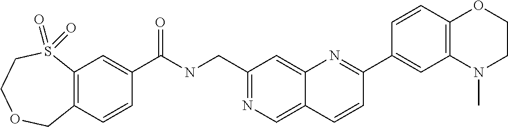 [SMILES: CN1CCOc2ccc(-c3ccc4cnc(CNC(=O)c5ccc6c(c5)S(=O)(=O)CCOC6)cc4n3)cc21] |
| 212 | 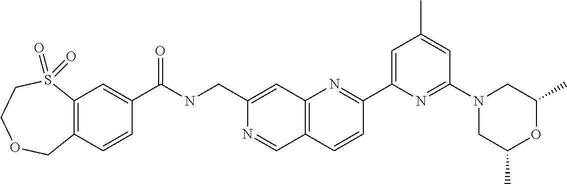 [SMILES: Cc1cc(-c2ccc3cnc(CNC(=O)c4ccc5c(c4)S(=O)(=O)CCOC5)cc3n2)nc(N2C[C@@H](C)O[C@@H](C)C2)c1] |
| 189 | 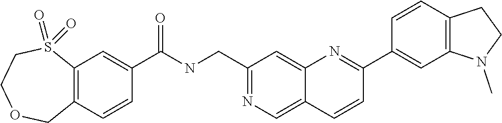 [SMILES: CN1CCc2ccc(-c3ccc4cnc(CNC(=O)c5ccc6c(c5)S(=O)(=O)CCOC6)cc4n3)cc21] |
| 175 | 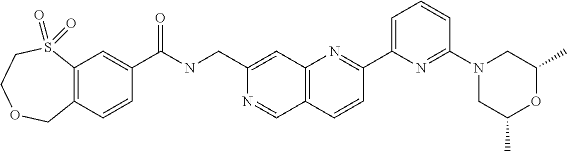 [SMILES: C[C@@H]1CN(c2cccc(-c3ccc4cnc(CNC(=O)c5ccc6c(c5)S(=O)(=O)CCOC6)cc4n3)n2)C[C@H](C)O1] |
| 249 | 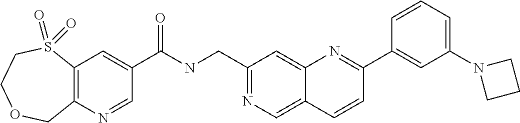 [SMILES: O=C(NCc1cc2nc(-c3cccc(N4CCC4)c3)ccc2cn1)c1cnc2c(c1)S(=O)(=O)CCOC2] |
| 210 | 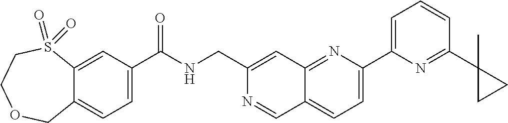 [SMILES: CC1(c2cccc(-c3ccc4cnc(CNC(=O)c5ccc6c(c5)S(=O)(=O)CCOC6)cc4n3)n2)CC1] |
| 246 | 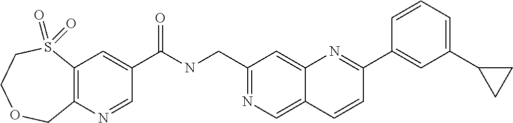 [SMILES: O=C(NCc1cc2nc(-c3cccc(C4CC4)c3)ccc2cn1)c1cnc2c(c1)S(=O)(=O)CCOC2] |
| 208 | 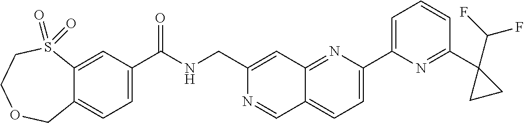 [SMILES: O=C(NCc1cc2nc(-c3cccc(C4(C(F)F)CC4)n3)ccc2cn1)c1ccc2c(c1)S(=O)(=O)CCOC2] |
| 262 | 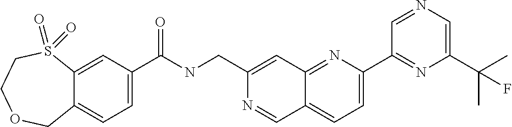 [SMILES: CC(C)(F)c1cncc(-c2ccc3cnc(CNC(=O)c4ccc5c(c4)S(=O)(=O)CCOC5)cc3n2)n1] |
| 204 | 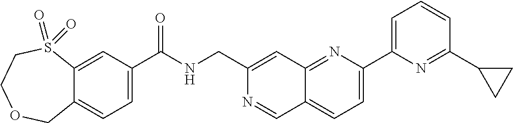 [SMILES: O=C(NCc1cc2nc(-c3cccc(C4CC4)n3)ccc2cn1)c1ccc2c(c1)S(=O)(=O)CCOC2] |
| 209 | 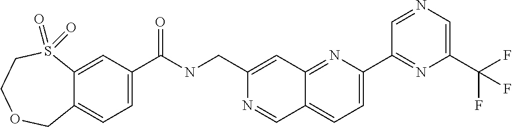 [SMILES: O=C(NCc1cc2nc(-c3cncc(C(F)(F)F)n3)ccc2cn1)c1ccc2c(c1)S(=O)(=O)CCOC2] |
| 285 | 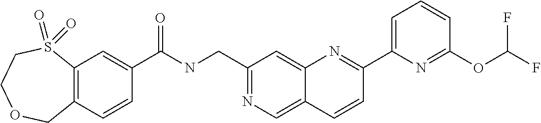 [SMILES: O=C(NCc1cc2nc(-c3cccc(OC(F)F)n3)ccc2cn1)c1ccc2c(c1)S(=O)(=O)CCOC2] |
| 305 | 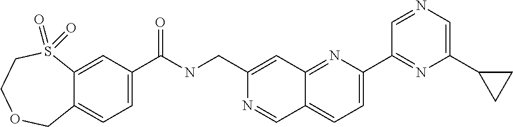 [SMILES: O=C(NCc1cc2nc(-c3cncc(C4CC4)n3)ccc2cn1)c1ccc2c(c1)S(=O)(=O)CCOC2] |
| 291 | 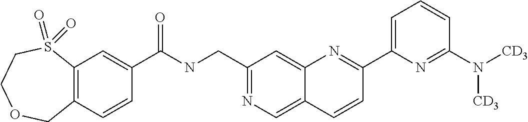 [SMILES: [2H]C([2H])([2H])N(c1cccc(-c2ccc3cnc(CNC(=O)c4ccc5c(c4)S(=O)(=O)CCOC5)cc3n2)n1)C([2H])([2H])[2H]] |
| 182 | 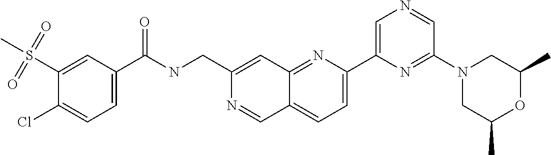 |
| 276 | 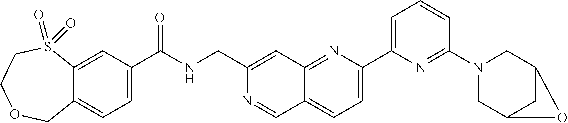 [SMILES: O=C(NCc1cc2nc(-c3cccc(N4CC5CC(C4)O5)n3)ccc2cn1)c1ccc2c(c1)S(=O)(=O)CCOC2] |
| 163 | 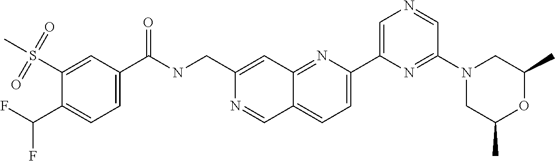 |
| 274 | 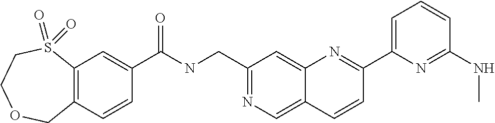 [SMILES: CNc1cccc(-c2ccc3cnc(CNC(=O)c4ccc5c(c4)S(=O)(=O)CCOC5)cc3n2)n1] |
| 230 | 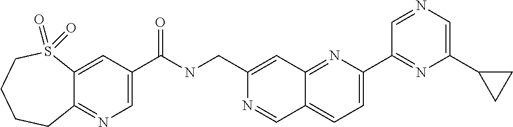 [SMILES: O=C(NCc1cc2nc(-c3cncc(C4CC4)n3)ccc2cn1)c1cnc2c(c1)S(=O)(=O)CCCC2] |
| 264 | 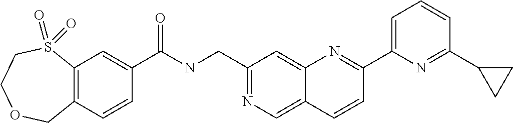 [SMILES: O=C(NCc1cc2nc(-c3cccc(C4CC4)n3)ccc2cn1)c1ccc2c(c1)S(=O)(=O)CCOC2] |
| 236 | 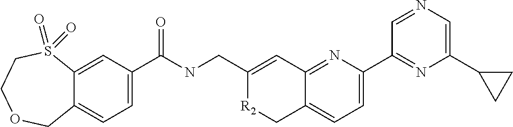 |
| 289 | 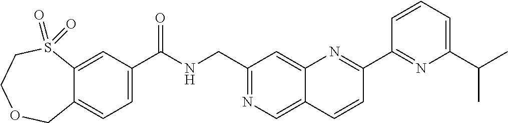 [SMILES: CC(C)c1cccc(-c2ccc3cnc(CNC(=O)c4ccc5c(c4)S(=O)(=O)CCOC5)cc3n2)n1] |
| 269 | 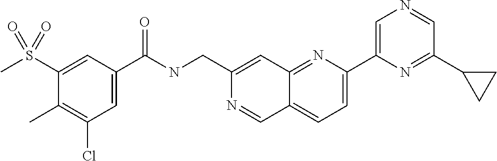 [SMILES: Cc1c(Cl)cc(C(=O)NCc2cc3nc(-c4cncc(C5CC5)n4)ccc3cn2)cc1S(C)(=O)=O] |
| 281 | 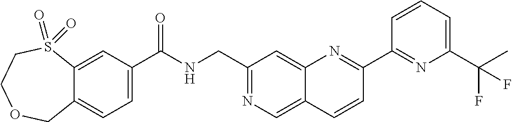 [SMILES: CC(F)(F)c1cccc(-c2ccc3cnc(CNC(=O)c4ccc5c(c4)S(=O)(=O)CCOC5)cc3n2)n1] |
| 272 | 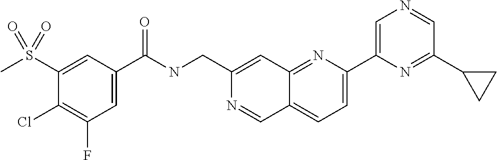 [SMILES: CS(=O)(=O)c1cc(C(=O)NCc2cc3nc(-c4cncc(C5CC5)n4)ccc3cn2)cc(F)c1Cl] |
| 280 | 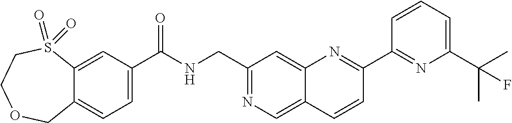 [SMILES: CC(C)(F)c1cccc(-c2ccc3cnc(CNC(=O)c4ccc5c(c4)S(=O)(=O)CCOC5)cc3n2)n1] |
| 298 | 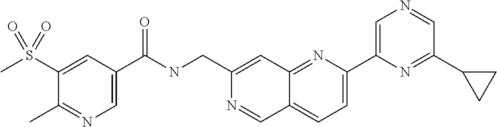 [SMILES: Cc1ncc(C(=O)NCc2cc3nc(-c4cncc(C5CC5)n4)ccc3cn2)cc1S(C)(=O)=O] |
| 241 | 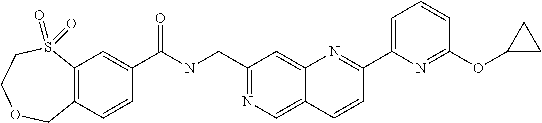 [SMILES: O=C(NCc1cc2nc(-c3cccc(OC4CC4)n3)ccc2cn1)c1ccc2c(c1)S(=O)(=O)CCOC2] |
| 302 | 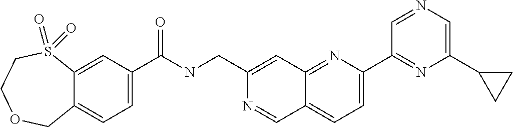 [SMILES: O=C(NCc1cc2nc(-c3cncc(C4CC4)n3)ccc2cn1)c1ccc2c(c1)S(=O)(=O)CCOC2] |
| 239 | 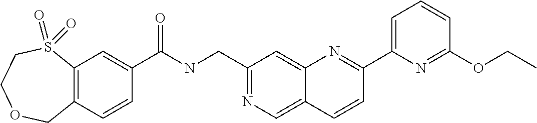 [SMILES: CCOc1cccc(-c2ccc3cnc(CNC(=O)c4ccc5c(c4)S(=O)(=O)CCOC5)cc3n2)n1] |
| 288 | 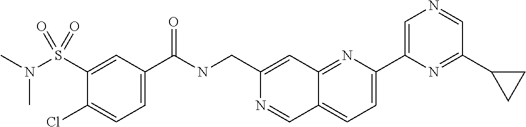 [SMILES: CN(C)S(=O)(=O)c1cc(C(=O)NCc2cc3nc(-c4cncc(C5CC5)n4)ccc3cn2)ccc1Cl] |
| 237 | 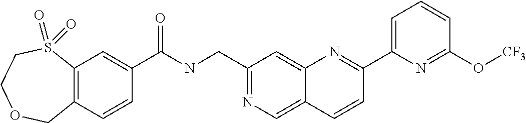 [SMILES: O=C(NCc1cc2nc(-c3cccc(OC(F)(F)F)n3)ccc2cn1)c1ccc2c(c1)S(=O)(=O)CCOC2] |
| 188 | 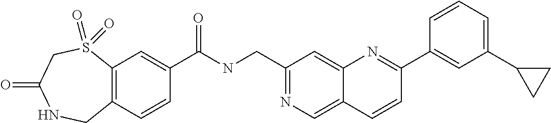 [SMILES: O=C1CS(=O)(=O)c2cc(C(=O)NCc3cc4nc(-c5cccc(C6CC6)c5)ccc4cn3)ccc2CN1] |
| 183 | 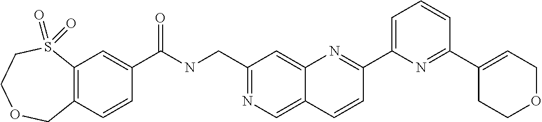 [SMILES: O=C(NCc1cc2nc(-c3cccc(C4=CCOCC4)n3)ccc2cn1)c1ccc2c(c1)S(=O)(=O)CCOC2] |
| 197 | 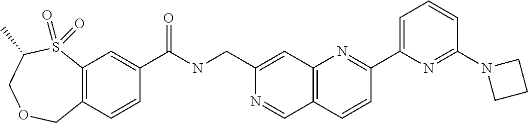 [SMILES: C[C@H]1COCc2ccc(C(=O)NCc3cc4nc(-c5cccc(N6CCC6)n5)ccc4cn3)cc2S1(=O)=O] |
| 169 | 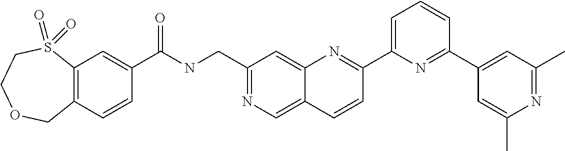 [SMILES: Cc1cc(-c2cccc(-c3ccc4cnc(CNC(=O)c5ccc6c(c5)S(=O)(=O)CCOC6)cc4n3)n2)cc(C)n1] |
| 199 | 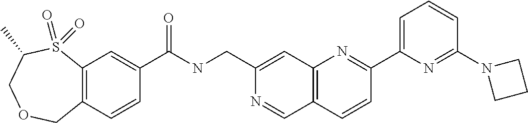 [SMILES: C[C@H]1COCc2ccc(C(=O)NCc3cc4nc(-c5cccc(N6CCC6)n5)ccc4cn3)cc2S1(=O)=O] |
| 213 | 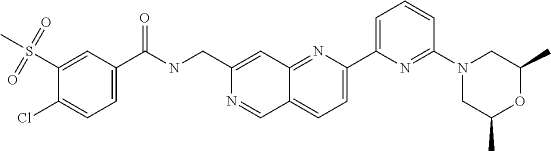 |
| 287 | 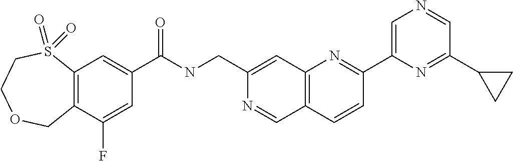 [SMILES: O=C(NCc1cc2nc(-c3cncc(C4CC4)n3)ccc2cn1)c1cc(F)c2c(c1)S(=O)(=O)CCOC2] |
| 206 | 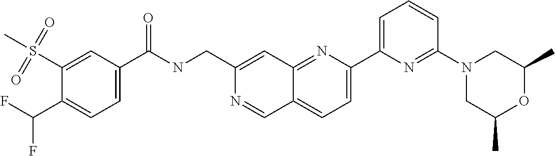 |
| 187 | 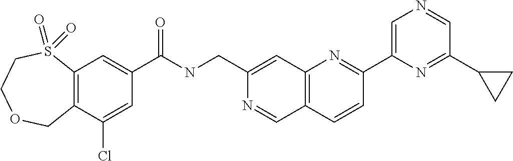 [SMILES: O=C(NCc1cc2nc(-c3cncc(C4CC4)n3)ccc2cn1)c1cc(Cl)c2c(c1)S(=O)(=O)CCOC2] |
| 195 | 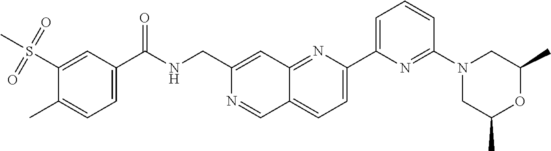 |
| 293 | 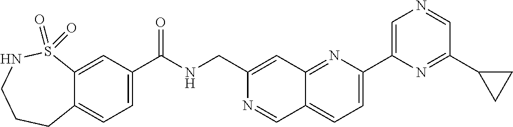 [SMILES: O=C(NCc1cc2nc(-c3cncc(C4CC4)n3)ccc2cn1)c1ccc2c(c1)S(=O)(=O)NCCC2] |
| 192 | 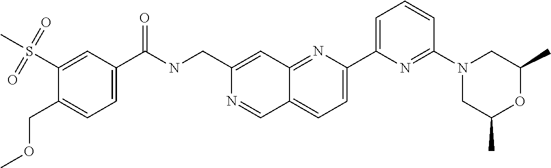 |
| 297 | 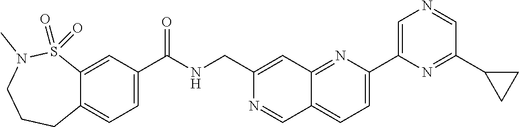 [SMILES: CN1CCCc2ccc(C(=O)NCc3cc4nc(-c5cncc(C6CC6)n5)ccc4cn3)cc2S1(=O)=O] |
| 180 | 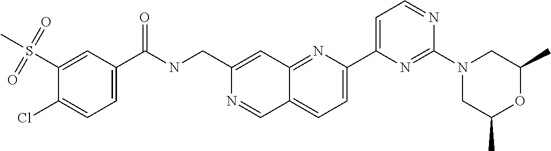 |
| 252 | 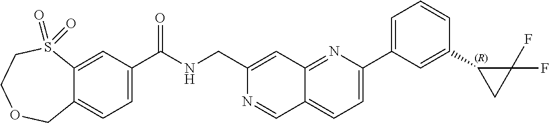 |
| 157 | 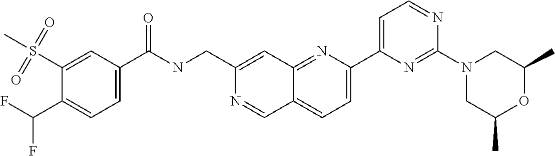 [SMILES: C[C@@H]1CN(c2nccc(-c3ccc4cnc(CNC(=O)c5ccc(C(F)F)c(S(C)(=O)=O)c5)cc4n3)n2)C[C@H](C)O1] |
| 251 | 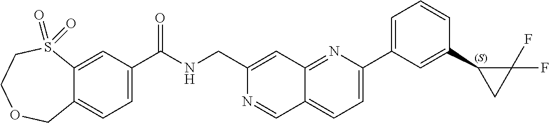 |
| 225 | 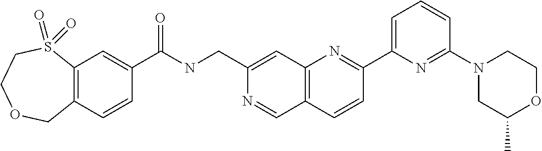 [SMILES: C[C@@H]1CN(c2cccc(-c3ccc4cnc(CNC(=O)c5ccc6c(c5)S(=O)(=O)CCOC6)cc4n3)n2)CCO1] |
| 270 | 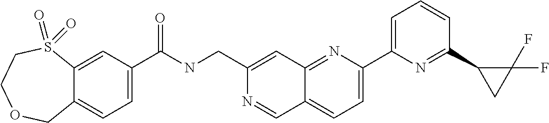 [SMILES: O=C(NCc1cc2nc(-c3cccc([C@@H]4CC4(F)F)n3)ccc2cn1)c1ccc2c(c1)S(=O)(=O)CCOC2] |
| 224 | 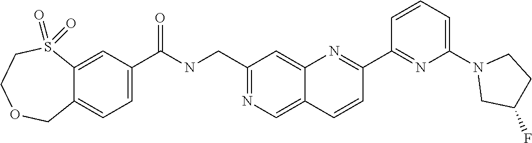 [SMILES: O=C(NCc1cc2nc(-c3cccc(N4CC[C@H](F)C4)n3)ccc2cn1)c1ccc2c(c1)S(=O)(=O)CCOC2] |
| 267 | 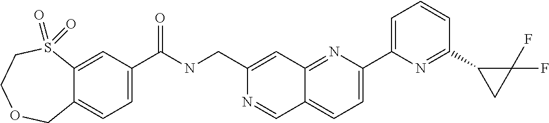 [SMILES: O=C(NCc1cc2nc(-c3cccc([C@H]4CC4(F)F)n3)ccc2cn1)c1ccc2c(c1)S(=O)(=O)CCOC2] |
| 223 | 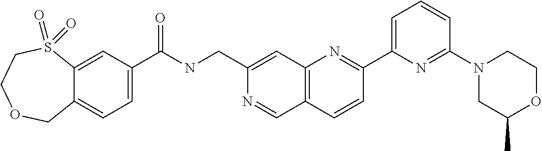 [SMILES: C[C@H]1CN(c2cccc(-c3ccc4cnc(CNC(=O)c5ccc6c(c5)S(=O)(=O)CCOC6)cc4n3)n2)CCO1] |
| 217 | 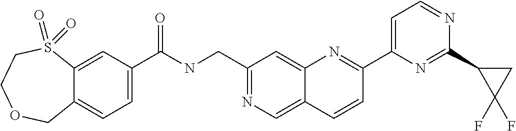 [SMILES: O=C(NCc1cc2nc(-c3ccnc([C@H]4CC4(F)F)n3)ccc2cn1)c1ccc2c(c1)S(=O)(=O)CCOC2] |
| 222 | 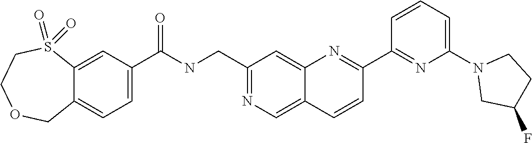 [SMILES: O=C(NCc1cc2nc(-c3cccc(N4CC[C@@H](F)C4)n3)ccc2cn1)c1ccc2c(c1)S(=O)(=O)CCOC2] |
| 218 | 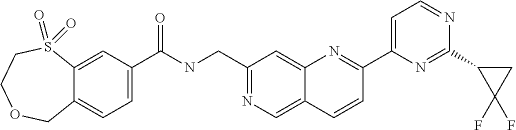 [SMILES: O=C(NCc1cc2nc(-c3ccnc([C@@H]4CC4(F)F)n3)ccc2cn1)c1ccc2c(c1)S(=O)(=O)CCOC2] |
| 221 | 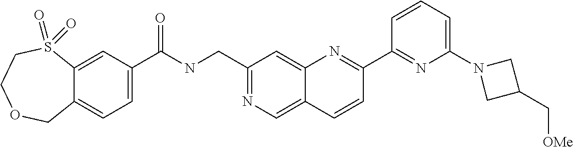 [SMILES: COCC1CN(c2cccc(-c3ccc4cnc(CNC(=O)c5ccc6c(c5)S(=O)(=O)CCOC6)cc4n3)n2)C1] |
| 219 | 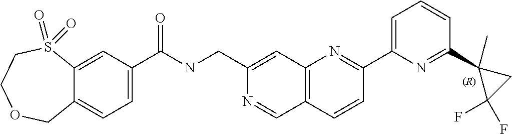 |
| 220 | 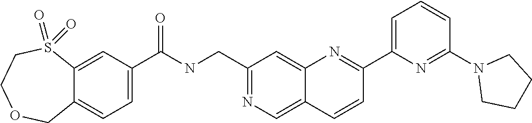 [SMILES: O=C(NCc1cc2nc(-c3cccc(N4CCCC4)n3)ccc2cn1)c1ccc2c(c1)S(=O)(=O)CCOC2] |
| 214 | 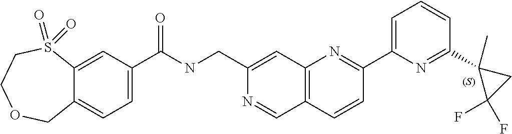 |
| 216 | 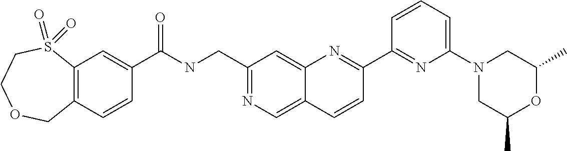 [SMILES: C[C@H]1CN(c2cccc(-c3ccc4cnc(CNC(=O)c5ccc6c(c5)S(=O)(=O)CCOC6)cc4n3)n2)C[C@H](C)O1] |
| 286 | 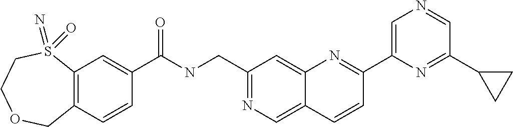 [SMILES: N=S1(=O)CCOCc2ccc(C(=O)NCc3cc4nc(-c5cncc(C6CC6)n5)ccc4cn3)cc21] |
| 215 | 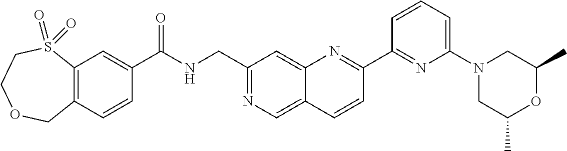 [SMILES: C[C@@H]1CN(c2cccc(-c3ccc4cnc(CNC(=O)c5ccc6c(c5)S(=O)(=O)CCOC6)cc4n3)n2)C[C@@H](C)O1] |
| 37 | 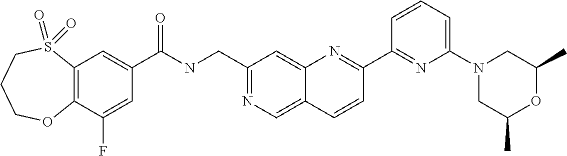 [SMILES: C[C@@H]1CN(c2cccc(-c3ccc4cnc(CNC(=O)c5cc(F)c6c(c5)S(=O)(=O)CCCO6)cc4n3)n2)C[C@H](C)O1] |
| 202 | 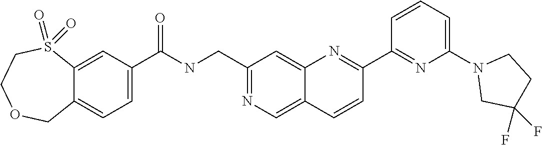 [SMILES: O=C(NCc1cc2nc(-c3cccc(N4CCC(F)(F)C4)n3)ccc2cn1)c1ccc2c(c1)S(=O)(=O)CCOC2] |
| 15 | 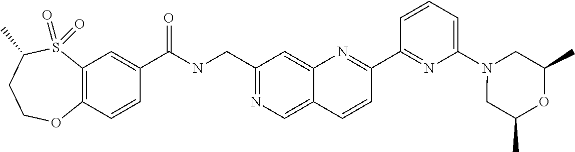 |
| 201 | 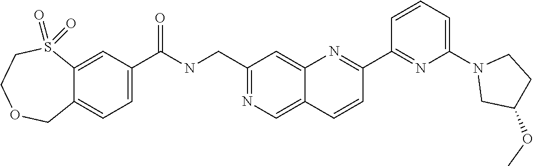 [SMILES: CO[C@H]1CCN(c2cccc(-c3ccc4cnc(CNC(=O)c5ccc6c(c5)S(=O)(=O)CCOC6)cc4n3)n2)C1] |
| 13 | 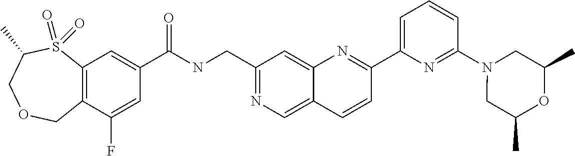 |
| 200 | 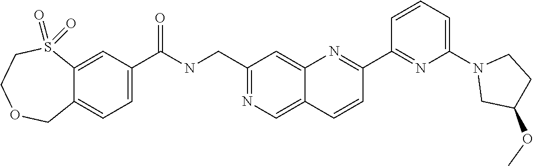 [SMILES: CO[C@@H]1CCN(c2cccc(-c3ccc4cnc(CNC(=O)c5ccc6c(c5)S(=O)(=O)CCOC6)cc4n3)n2)C1] |
| 101 | 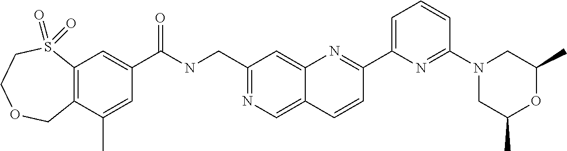 [SMILES: Cc1cc(C(=O)NCc2cc3nc(-c4cccc(N5C[C@@H](C)O[C@@H](C)C5)n4)ccc3cn2)cc2c1COCCS2(=O)=O] |
| 308 | 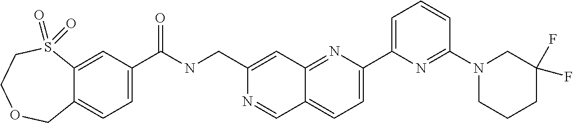 [SMILES: O=C(NCc1cc2nc(-c3cccc(N4CCCC(F)(F)C4)n3)ccc2cn1)c1ccc2c(c1)S(=O)(=O)CCOC2] |
| 63 | 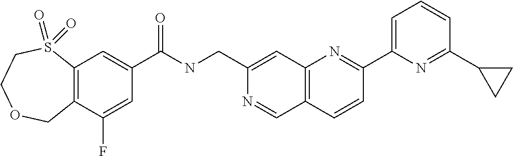 [SMILES: O=C(NCc1cc2nc(-c3cccc(C4CC4)n3)ccc2cn1)c1cc(F)c2c(c1)S(=O)(=O)CCOC2] |
| 186 | 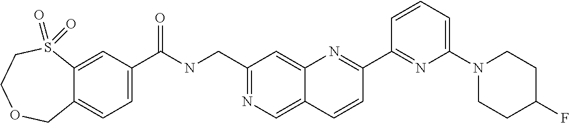 [SMILES: O=C(NCc1cc2nc(-c3cccc(N4CCC(F)CC4)n3)ccc2cn1)c1ccc2c(c1)S(=O)(=O)CCOC2] |

### Materials

Unless otherwise noted, all materials were obtained from commercial suppliers and were used without further purification. All reactions involving air- or moisture-sensitive reagents were performed under a nitrogen atmosphere.

### Example 1. Preparation of Intermediates

### Intermediate 1. 2,3-Dihydro-6H-benzo[e][1,4]oxathiepine-8-carboxylic acid 1,1-dioxide

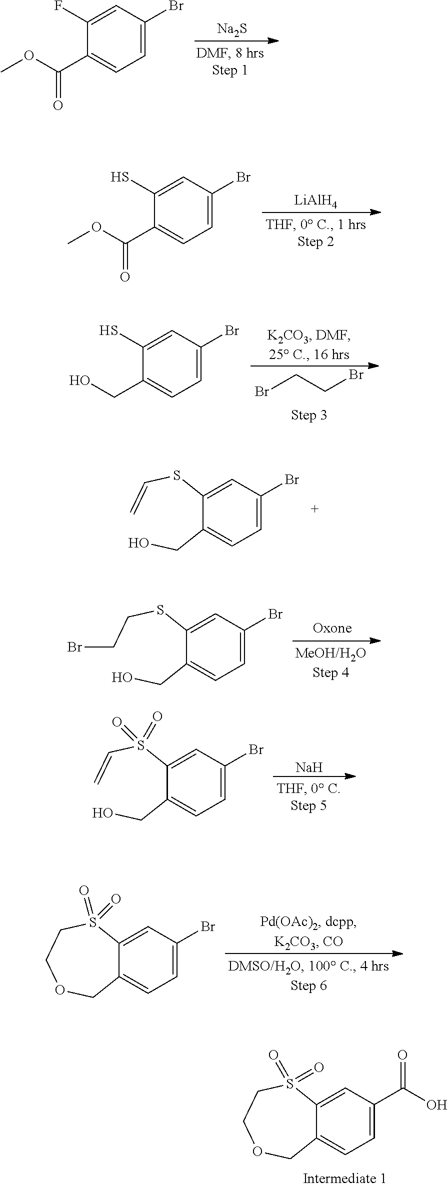

### Step 1: Preparation of methyl 4-bromo-2-mercaptobenzoate

To a solution of methyl 4-bromo-2-fluoro-benzoate (100 g, 429.12 mmol) in DMF (1 L) was added sodium sulfide (33.49 g, 429.1 mmol, 18.0 mL) and the mixture was stirred at 30° C.; for 16 h. The mixture was poured into water (6000 mL) and then was adjusted pH to ˜3 with 2N HCl. The mixture was extracted with MTBE (3000 mL×2). The combined organic phase was washed with brine (3000 mL×3), dried over anhydrous Na₂SO₄, filtered and concentrated under vacuum to give methyl 4-bromo-2-mercaptobenzoate (103 g, crude) as yellow oil, which was used for the next step without further purification.

¹H NMR (400 MHz, DMSO_d6) δ=7.91 (d, J=1.6 Hz, 1H), 7.83-7.81 (m, 1H), 7.43-7.40 (m, 1H), 5.58 (br s, 1H), 3.83 (s, 3H) ppm

### Step 2: Preparation of (4-bromo-2-mercaptophenyl)methanol

To a mixture of methyl 4-bromo-2-mercaptobenzoate (103 g, 416.82 mmol) in THE (1000 mL) was added LiAlH₄ (15.82 g, 418.82 mmol) at 0° C. under N₂. The mixture was stirred at 0° C. for 1 hr. The mixture was poured into 1N HCl (2000 mL) and extracted with EtOAc (2000 mL×2). The combined organic phase was washed with brine (2000 mL), dried over anhydrous Na₂SO₄, filtered and concentrated under vacuum to give (4-bromo-2-mercaptophenyl)methanol (88 g. crude) as yellow oil, which was used for the next step without further purification.

¹H NMR (400 MHz, DMSO_d6) δ=7.59 (s, 1H), 7.32 (d, J=1.2 Hz, 2H), 5.56-5.36 (m, 2H), 4.39 (s, 2H) ppm

### Step 3: Preparation of (4-bromo-2-(vinylthio)phenyl)methanol and (4-bromo-2-((2-bromoethyl)thio)phenyl)methanol

To a mixture of (4-bromo-2-mercaptophenyl)methanol (85 g, 387.95 mmol) in DMF (1700 mL) was added K₂CO₂ (160.9 g, 1.16 mol) and 1,2-dibromoethane (218.6 g, 1.16 mol, 87.8 mL) and the mixture was stirred at 25° C. for 1 hr. Then the mixture was stirred at 70° C. for another 24 h. The reaction mixture was poured into Sat·NH₄Cl (10 L) and extracted with EA (3000 mL*2). The combined organics were washed with brine (4000 mL×2), dried over Na₂SO₄, filtered and filtrate was evaporated to dryness. The residue was purified by silica gel column chromatography (PE/EA=50/1 to 5/1). The fraction was concentrated in vacuum to give (4-bromo-2-(vinylthio)phenyl)methanol (33.5 g, 136.68 mmol, 35% yield) and (4-bromo-2-((2-bromoethyl)thio)phenyl)methanol (10 g, 30.67 mmol, 8% yield) as yellow oil. (4-bromo-2-(vinylthio)phenyl)methanol:

¹H NMR (400 MHz, CDCl₃) δ=7.55 (s, 1H), 7.45 (d, J=2 Hz, 1H), 7.43 (d, J=2 Hz, 1H), 6.49-8.42 (m, 1H), 5.45 (d, J=9.8 Hz, 1H), 5.32 (d, J=10.4 Hz, 1H), 4.73 (s, 2H) ppm. (4-bromo-2-((2-bromoethyl)thio)phenyl)methanol:

¹H NMR (400 MHz, CDCl₃) δ=7.46 (s, 1H), 7.33 (d, J=2 Hz, 1H), 7.09 (d, J=2 Hz, 1H), 6.67 (s, 2H), 3.41-3.38 (m, 2H), 3.25-3.23 (m, 1H) ppm.

### Step 4: Preparation of (4-bromo-2-(vinylsulfonyl)phenyl)methanol

To a mixture of (4-bromo-2-(vinylthio)phenyl)methanol (35.5 g, 144.82 mmol) in MeOH (350 mL) and H₂O (350 mL) was added Oxone® (133.54 g, 217.23 mmol) and the mixture was stirred at 25° C. for 2 h. Water (1500 mL) was added and the mixture was extracted with EtOAc (1500 mL×2). The combined organic phase was washed with brine (1000 mL×2), dried over anhydrous Na₂SO₄, filtered and concentrated under vacuum to give (4-bromo-2-vinylsulfonyl-phenyl)methanol (38.5 g, crude) as a yellow solid, which was used for the next step without further purification.

¹H NMR (400 MHz, DMSO_d6) δ=7.98-7.95 (m, 2H), 7.77-7.75 (m, 1H), 7.22-7.15 (m, 1H), 6.43-6.39 (m, 1H), 6.31 (d, J=10.0 Hz, 1H), 5.62-5.59 (m, 1H), 4.75 (d, J=5.2 Hz, 2H) ppm

### Step 5: Preparation of 8-bromo-2,3-dihydro-5H-benzo[e][1,4]oxathiepine 1,1-dioxide

To a mixture of (4-bromo-2-vinylsulfonyl-phenyl)methanol (38.5 g, 138.9 mmol) in DMF (1000 mL) was added NaH (11.11 g, 277.84 mmol, 60% purity) at 0° C. under N₂. The mixture was stirred at 0° C. for 1 hr. The reaction mixture was poured into sat. NH₄Cl (2 L) and extracted with EA (2000 mL*2). The combined organic phase was washed with brine (2000 mL), dried over anhydrous Na₂SO₄, filtered, and concentrated under vacuum. The residue was purified by column chromatography (SiO₂, PE:EtOAc=50:1-5:1) and concentrated in vacuum to give 8-bromo-2,3-dihydro-5H-benzo[e][1,4]oxathiepine 1,1-dioxide (26.5 g, 95.62 mmol, 69% yield) as a white solid.

¹H NMR (400 MHz, DMSO_d6) δ=7.99 (d, J=2.0 Hz, 1H), 7.92-7.90 (m, 1H), 7.55 (d, J=8.0 Hz, 1H), 4.88 (s, 2H), 4.20-4.17 (m, 2H), 3.88-3.68 (m, 2H) ppm

### Step 6: Preparation of 2,3-dihydro-6H-benzo[e][1,4]oxathiepine-8-carboxylic acid 1,1-dioxide (Intermediate 1)

To a mixture of 8-bromo-2,3-dihydro-5H-benzo[e][1,4]oxathiepine 1,1-dioxide (8.8 g, 31.75 mmol) in DMSO (90 mL) and H₂O (9 mL) was added 1,3-bis(dicyclohexylphosphino)propane bis(tetrafluoroborate) (3.89 g, 8.35 mmol), K₂CO₃ (6.58 g, 47.63 mmol) and Pd(OAc)₂ (712.90 mg, 3.18 mmol). The mixture was purged with CO for three times and then was stirred at 100° C. under CO (15 psi) for 4 h. Water (3000 mL) was added and the mixture was extracted with EtOAc (500 mL×2) and then the organic phase was discarded. The aqueous layer was adjusted pH to ˜3 with 1N HCl. Then the mixture was extracted with EA (500 mL*5). The combined organic phase was washed with brine (2000 mL), dried over anhydrous Na₂SO₄, filtered and concentrated under vacuum. The crude was washed by MTBE (20 mL*2), then filtered, the filter cake was evaporated to dryness to give 2,3-dihydro-5H-benzo[e][1,4]oxathiepine-8-carboxylic acid 1,1 dioxide (15 g, 61.92 mmol, 65% yield) as a white solid.

¹H NMR (400 MHz, DMSO-d6) δ=8.43 (s, 1H), 8.20-8.18 (m, 1H), 7.72-7.70 (m, 1H), 4.96 (s, 2H), 4.23-4.20 (m, 2H), 3.67-3.66 (m, 2H) ppm.

### Intermediate 2. 3,5-Dihydro-2H-[1,4]oxathiepino[6,5-b]pyridine-8-carboxylic acid 1,1-dioxide

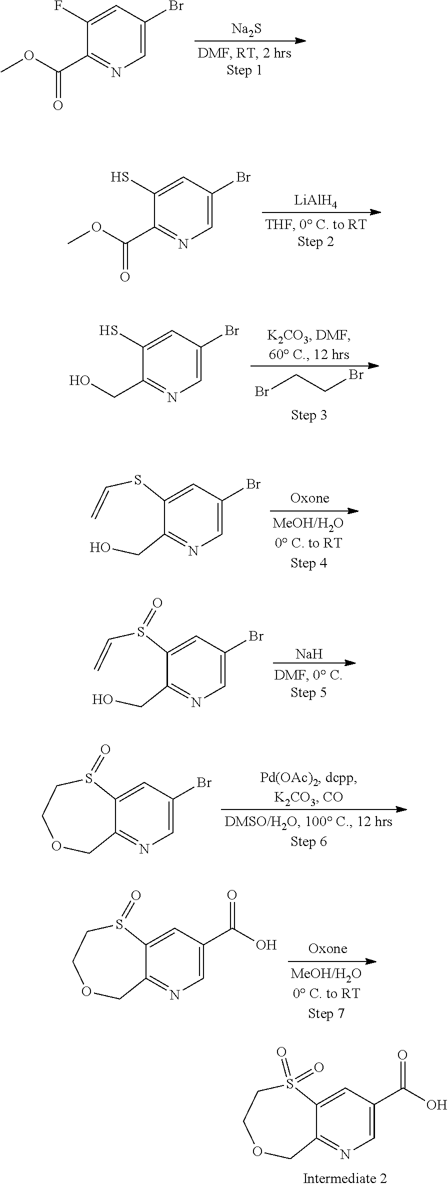

### Step 1: Preparation of methyl 5-bromo-3-mercaptopicolinate

To a solution of methyl 5-bromo-3-fluoro-pyridine-2-carboxylate (1 g, 4.27 mmol) in DMF (10 mL) was added Na₂S (333.49 mg, 4.27 mmol). The mixture was stirred at 25° C. for 2 h. Three of the same batches were combined and purified together. The mixture was diluted with water (50 mL) and adjusted to pH=5 with 1N aq. HCl. The mixture was extracted with EA (50 mL×2). The combined organic layer was washed by brine (50 mL×2), dried with anhydrous Na₂SO₄ and concentrated to afford methyl 5-bromo-3-mercaptopicolinate (3.3 g, crude) as a brown oil. LCMS (ESI) m/z: [M+H]⁺=247.8/249.8

### Step 2: Preparation of (5-bromo-3-mercaptopyridin-2-yl)methanol

To a solution of methyl 5-bromo-3-mercaptopicolinate (3.3 g, 13.30 mmol) in THE (33 mL) was added LiAlH₄ (504.8 mg, 13.30 mmol) at 0° C. The mixture was stirred at 25° C. for 2 h. The mixture was diluted with water (100 mL) and adjusted to pH=8 with 1N aq. HCl. Then the mixture was extracted with EA (100 mL×2). The combined organic layer was dried over anhydrous Na₂SO₄ and concentrated to afford (5-bromo-3-mercaptopyridin-2-yl)methanol (1.68 g, 7.54 mmol) as a brown oil. LCMS (ESI) m/z: [M+H]⁺=219.8/221.8.

### Step 3: Preparation of (5-bromo-3-(vinylthio)pyridin-2-yl)methanol

To a solution of (5-bromo-3-mercaptopyridin-2-yl)methanol (1.66 g, 7.54 mmol) in DMF (15 mL) was added K₂CO₃ (3.13 g, 22.63 mmol) and 1,2-dibromoethane (7.08 g, 37.71 mmol, 2.85 mL). The mixture was stirred at 80° C. for 12 h. The mixture was diluted with water (100 mL) and extracted with EA (100 mL×2). The combined organic layer was dried with anhydrous Na₂SO₄ and concentrated to afford residue. The residue was purified by flash silica gel chromatography (ISCO®; 20 g SepaFlash® Silica Flash Column, Eluent of 0˜100% Ethylacetate/Petroleum ether). The eluent was concentrated to afford (5-bromo-3-(vinylthio)pyridin-2-yl)methanol (800 mg, 2.44 mmol, 32% yield) as a brown oil.

LCMS (ESI) m/z: [M+H]⁺=245.9/247.9.

¹HNMR (400 MHz, DMSO-d₆) δ=8.53 (d, J=2.0 Hz, 1H), 7.92 (d, J=2.0 Hz, 1H), 6.81-6.74 (m, 1H), 5.64-5.51 (m, 2H), 5.32-5.29 (m, 1H), 4.54 (d, J=6.0 Hz, 2H) ppm.

### Step 4: Preparation of (5-bromo-3-(vinylsulfinyl)pyridin-2-yl)methanol

To a solution of (5-bromo-3-(vinylthio)pyridin-2-yl)methanol (600 mg, 2.44 mmol) in MeOH (6 mL) was added Oxone® (824.27 mg, 1.34 mmol) in water (8 mL) slowly at 0° C. The mixture was stirred at 25° C. for 1 hr. The mixture was quenched by saturated aq·Na₂SO₃ (30 mL) and extracted with EA (30 mL×2). The combined organic layer was dried over anhydrous Na₂SO₄ and concentrated to afford residue. The residue was purified by flash silica gel chromatography (ISCO®; 12 g SepaFlash® Silica Flash Column, Eluent of 0˜100% Ethylacetate/Petroleum ether). The eluent was concentrated to afford (5-bromo-3-(vinylsulfinyl)pyridin-2-yl)methanol (500 mg, 1.91 mmol, 78.25% yield) as a colorless oil.

¹HNMR (400 MHz, DMSO-d₆) δ=8.74 (d, J=2.4 Hz, 1H), 8.17 (d, J=2.0 Hz, 1H), 7.18-7.12 (m, 1H), 6.09-6.02 (m, 2H), 5.95 (d, J=9.6 Hz, 1H), 4.85-4.78 (m, 1H), 4.73-4.66 (m, 1H) ppm.

### Step 6: Preparation of 8-bromo-3,5-dihydro-2H-[1,4]oxathiepino[6,5-b]pyridine 1-oxide

To a solution of (5-bromo-3-(vinylsulfinyl)pyridin-2-yl)methanol (500 mg, 1.91 mmol) In DMF (5 mL) was added NaH (152.59 mg, 3.81 mmol, 60% purity) at 0° C. The mixture was stirred at 0° C. for 2 h. The mixture was quenched by saturated aq. NH₄Cl (30 mL) and extracted with EA (30 mL×2). The combined organic layer was dried over anhydrous Na₂SO₄ and concentrated to afford residue. The residue was purified by flash silica gel chromatography (ISCO®; 12 g SepaFlash® Silica Flash Column, Eluent of 0˜10% Ethylacetate/Petroleum ether). The eluent was concentrated to afford 8-bromo-3,5-dihydro-2H-[1,4]oxathiepino[6,5-b]pyridine 1-oxide (350 mg, 1.34 mmol, 70% yield) as a colorless oil.

¹HNMR (400 MHz, DMSO-d₆) δ=8.75 (d, J=2.4 Hz, 1H), 8.19 (d, J=2.0 Hz, 1H), 4.91-4.74 (m, 2H), 4.43-4.34 (m, 1H), 4.21-4.18 (m, 1H), 3.65-3.58 (m, 1H), 3.49-3.44 (m, 1H) ppm.

### Step 6: Preparation of 3,5-dihydro-2H-[1,4]oxathiepino[6,5-b]pyridine-8-carboxylic acid 1-oxide

To a solution of 8-bromo-3,5-dihydro-2H-[1,4]oxathiepino[6,5-b]pyridine 1-oxide (350 mg, 1.34 mmol) in DMSO (4 mL) and water (120.27 mg, 6.68 mmol, 120.27 uL) was added 1,3 bis(dicyclohexylphosphino)propane bis(tetrafluoroborate) (81.75 mg, 133.52 μmol), K₂CO₃ (276.82 mg, 2.00 mmol) and Pd(OAc)₂ (29.98 mg, 133.52 μmol). The mixture was degassed and purged with CO for 3 times. The mixture was stirred at 100° C. for 12 h under CO (15 psi) atmosphere. The mixture was filtered and washed by DMSO (2 mL) and water (2 mL). Then the filter liquid was adjusted to pH=6 with 1N aq. HCl. The filter liquid was purified by reversed-phase HPLC (0.1% FA condition). The eluent was concentrated to remove ACN and lyophilized to afford 3,5-dihydro-2H-[1,4]oxathiepino[6,5-b]pyridine-8-carboxylic acid 1-oxide (70 mg, 0.262 mmol, 20% yield) as a white solid.

LCMS (ESI) m/z: [M+H]⁺=227.9.

¹HNMR (400 MHz, DMSO-d₆) δ=9.03 (d, J=2.0 Hz, 1H), 8.51 (d, J=2.0 Hz, 1H), 5.01-4.88 (m, 2H), 4.43-4.41 (m, 1H), 4.18-4.15 (m, 1H), 3.83-3.62 (m, 2H), 3.52-3.48 (m, 2H) ppm.

### Step 7: Preparation of 3,5-dihydro-2H-[1,4]oxathiepino[6,5-b]pyridine-8-carboxylic acid 1,1-dioxide (Intermediate 2)

To a solution of 3,5-dihydro-2H-[1,4]oxathiepino[6,5-b]pyridine-8-carboxylic acid 1-oxide (70 mg, 0.309 mmol) in MeOH (0.7 mL) was added Oxone® (284.07 mg, 462.07 μmol) in water (0.7 mL) at 0° C. The mixture was stirred at 25° C. for 1 hr. The mixture was filtered. The filter cake was washed by MeOH (5 mL). Then the filter liquid was quenched by saturated Na₂SO₃ solution. Then the solution was purified by reversed-phase HPLC (0.1% FA condition). The eluent was concentrated to remove ACN and lyophilized to afford 3,5-dihydro-2H-[1,4]oxathiepino[6,5-b]pyridine-8-carboxylic acid 1,1-dioxide (38 mg, 138.16 μmol, 44% yield) as a white solid.

LCMS (ESI) m/z: [M+H]⁺=243.9.

### Intermediate 3. 3,4-Dihydro-2H-benzo[b][1,4]oxathiepine-7-carboxylic acid 5,6-dioxide

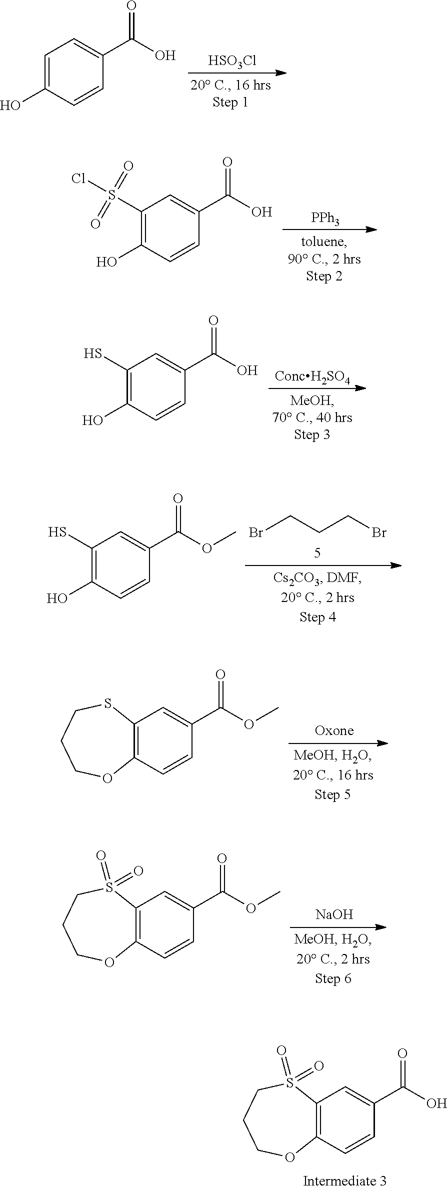

### Step 1: Preparation of 3-chlorosulfonyl-4-hydroxy-benzoic acid

To a solution of HSO₃Cl (31 mL) was added portionwise 4-hydroxybenzoic acid (5.5 g, 39.82 mmol). The mixture was stirred at 20° C. for 16 h. The reaction mixture was dropwise added slowly ice water (300 mL). The mixture was extracted with ethyl acetate (100 mL×3). The combined organic layers were washed with brine (50 mL), dried over Na₂SO₄, concentrated in vacuo to give a residue. The crude product was triturated with PE (30 mL) at 20° C. for 30 min to afford 3-chlorosulfonyl-4-hydroxy-benzoic acid (4.5 g, 13.72 mmol, 74% yield) as a white solid.

¹HNMR (400 MHz, DMSO-d₆) δ=8.09-8.06 (m, 1H), 7.82-7.75 (m, 1H), 6.89-6.81 (m, 1H) ppm.

### Step 2: Preparation of 4-hydroxy-3-mercaptobenzoic acid

To a solution of 3-chlorosulfonyl-4-hydroxy-benzoic acid (1 g, 4.23 mmol) in toluene (20 mL) was added PPh₃ (3.88 g, 14.79 mmol) in portions. The mixture was stirred at 90° C. for 2 h. The reaction was quenched by adding 10% NaOH solution (20 mL). The mixture was extracted with ethyl acetate (20 mL×3). The aqueous phase was adjusted to pH 2 with 1N HCl. The mixture was extracted with ethyl acetate (20 mL×3). The combined organic layers were dried over Na₂SO₄ and concentrated in vacuo to give 4-hydroxy-3-mercaptobenzoic acid (0.62 g, 3.64 mmol, 88.21% yield) as yellow oil.

¹H NMR (400 MHz, DMSO-d₆) δ=12.34 (s, 1H), 7.89-7.83 (m, 1H), 7.61-7.52 (m, 1H), 6.90-6.83 (m, 1H), 5.01 (s, 1H) ppm.

### Step 3: Preparation of methyl 4-hydroxy-3-mercaptobenzoate

To a solution of 4-hydroxy-3-mercaptobenzoic acid (0.6 g, 3.53 mmol) in MeOH (5 mL) was added dropwise H₂SO₄ (352.84 mg, 3.53 mmol, 191.76 uL, 98% purity). The mixture was stirred at 70° C. for 40 h. The reaction was quenched by adding water (20 mL). The mixture was extracted with ethyl acetate (20 mL*3). The combined organic layers were washed with brine (20 mL), dried over Na₂SO₄, concentrated in vacuo to give methyl 4-hydroxy-3-mercaptobenzoate (0.6 g, crude) as white solid.

¹H NMR (400 MHz, DMSO-d₆) δ=11.30 (s, 1H), 8.07-8.01 (m, 1H), 7.77-7.71 (m, 1H), 6.99-6.95 (m, 1H), 3.80-3.78 (m, 3H) ppm.

### Step 4: Preparation of methyl 3,4-dihydro-2H-benzo[b][1,4]oxathiepine-7-carboxylate

To a solution of methyl 4-hydroxy-3-mercaptobenzoate (0.1 g, 542.85 μmol, 1 eq) in DMF (5 mL) was added Cs₂CO₃ (884.36 mg, 2.71 mmol) and dropwise 1,3-dibromopropane (109.6 mg, 0.543 mmol, 55 uL). The mixture was stirred at 20° C. for 2 h. The reaction was quenched by adding water (20 mL). The mixture was extracted with ethyl acetate (20 ml×3). The combined organic layers were washed with brine (20 mL), dried over Na₂SO₄, concentrated in vacuo to give a residue. The residue was purified by prep-TLC (SiO₂, Petroleum ether:Ethyl acetate=1:1), the eluent was concentrated in vacuo to afford methyl 3,4-dihydro-2H-benzo[b][1,4]oxathiepine-7-carboxylate (65 mg, 0.274 mmol, 51% yield) as yellow oil.

LCMS (ESI) m/z: [M+H]⁺=225.1.

¹H NMR (400 MHz, CDCl₃) δ=8.09-8.02 (m, 1H), 7.83-7.74 (m, 1H), 7.03-6.94 (m, 1H), 4.45-4.34 (m, 2H), 3.93-3.84 (m, 3H), 3.10-2.98 (m, 2H), 2.34-2.22 (m, 2H) ppm.

### Step 5: Preparation of methyl 3,4-dihydro-2H-benzo[b][1,4]oxathiepine-7-carboxylate 5,5-dioxide

A mixture of methyl 3,4-dihydro-2H-1,5-benzoxathiepine-7-carboxylate (60 mg, 267.53 μmol) in MeOH (5 ml) and H₂O (5 mL) was added Oxone® (493.40 mg, 802.58 μmol), and then the mixture was stirred at 20° C. for 16 h. The reaction was quenched by adding Sat·Na₂SO₃ (30 mL). The mixture was extracted with DCM (30 mL×5). The combined organic layers were dried over Na₂SO₄ and concentrated in vacuo to give methyl 3,4-dihydro-2H-benzo[b][1,4]oxathiepine-7-carboxylate 5,5-dioxide (66 mg, 0.257 mmol, 96% yield) as yellow oil.

¹H NMR (400 MHz, CDCl3) δ=8.73-8.60 (m, 1H), 8.32-8.16 (m, 1H), 7.26-7.23 (m, 1H), 4.43-4.30 (m, 2H), 3.97-3.91 (m, 3H), 3.48-3.36 (m, 2H), 2.53-2.41 (m, 2H) ppm.

### Step 6: Preparation of 3,4-dihydro-2H-benzo[b][1,4]oxathiepine-7-carboxylic acid 5,5-dioxide (Intermediate 3)

A mixture of methyl 3,4-dihydro-2H-benzo[b][1,4]oxathiepine-7-carboxylate 5,5-dioxide (65 mg, 0.254 mmol) in MeOH (3 mL) and H₂O (3 mL) was added portionwise NaOH (30.44 mg, 0.761 mmol), and then the mixture was stirred at 20° C. for 2 h. The reaction was concentrated in vacuo to give a residue.

The residue was partitioned with EA (10 mL) and 1N NaOH solution (10 mL). The aqueous layer was adjusted to pH 1 with 1N HCl solution and extracted with EA (10 mL×3). The combined organic phases were concentrated in vacuo to give 3,4-dihydro-2H-benzo[b][1,4]oxathiepine-7-carboxylic acid 5,5-dioxide (80 mg, 0.248 mmol, 98% yield) as yellow solid.

LCMS (ESI) m/z: [M+Na]⁺=265.2.

¹H NMR (400 MHz, DMSO-d6) δ=8.42-8.30 (m, 1H), 8.21-8.10 (m, 1H), 7.41-7.29 (m, 1H), 4.34-4.22 (m, 2H), 2.31-2.22 (m, 2H), 1.81-1.71 (m, 2H) ppm.

### Intermediate 4. 6-Chloro-2,3-dihydro-5H-benzo[e][1,4]oxathiepine-8-carboxylic acid 1,1-dioxide

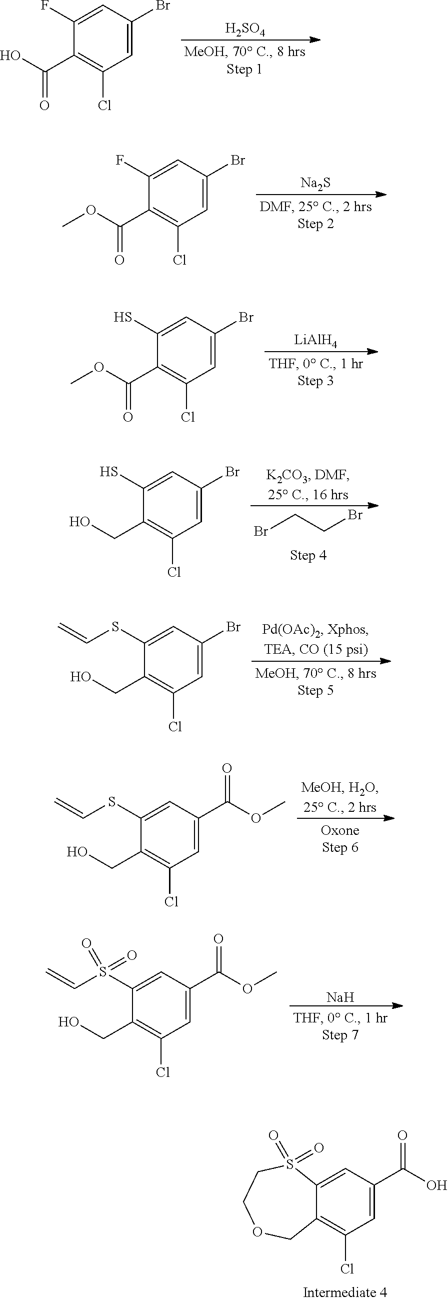

### Step 1: Preparation of methyl 4-bromo-2-chloro-6-fluorobenzoate

To a solution of 4-bromo-2-chloro-6-fluorobenzoic acid (10 g, 39.46 mmol) in MeOH (90 mL) was added conc. H₂SO₄ (18.4 g, 187.60 mmol, 10 mL) slowly, then the mixture was stirred at 70° C. for 8 h. The mixture was concentrated under vacuum to remove part of MeOH, then poured into sat. NaHCO₃ (200 mL), then extracted with EA (200 mL×2). The combined organic layers were washed with brine (100 mL×2), dried over Na₂SO₄, filtered and concentrated under reduced pressure to give methyl 4-bromo-2-chloro-6-fluorobenzoate (9.2 g. crude) as colorless oil, and used to next step directly.

¹H NMR (400 MHz, DMSO-d₆) δ=7.87-7.76 (m, 2H), 3.91 (s, 3H) ppm.

### Step 2: Preparation of methyl 4-bromo-2-chloro-6-mercaptobenzoate

To a solution of methyl methyl 4-bromo-2-chloro-8-fluorobenzoate (7.2 g, 26.92 mmol) in DMF (72 mL) was added Na₂S (2.10 g, 26.92 mmol), then the mixture was stirred at 25° C. for 2 h. The mixture was diluted with water (300 mL), then the resulting mixture was acidized to pH 3 with 1N HCl solution, extracted with EA (200 mL×2). The combined organic layers were washed with brine (250 mL×2), dried over Na₂SO₄, filtered and concentrated under reduced pressure to give methyl 4-bromo-2-chloro-6-mercaptobenzoate (7 g, crude) as yellow oil, the crude product was used to next step directly.

### Step 3: Preparation of (4-bromo-2-chloro-6-mercaptophenyl)methanol

To a mixture of methyl methyl 4-bromo-2-chloro-6-mercaptobenzoate (9 g, 31.97 mmol) in THE (90 mL) was added LiAlH₄ (1.33 g, 35.16 mmol) at 0° C., then the mixture was stirred at 0° C. for 1 hr. The mixture was poured into HCl (1 N, 200 mL), then extracted with EA (250 mL×2). The combined organic layers were washed with brine (200 mL×2), dried over Na₂SO₄, filtered and concentrated under reduced pressure to give (4-bromo-2-chloro-6-mercaptophenyl)methanol (5.8 g, crude) as a colorless oil

### Step 4: Preparation of (4-bromo-2-chloro-6-(vinylthio)phenyl)methanol

To a mixture of (4-bromo-2-chloro-6-mercaptophenyl)methanol (5.7 g, 22.48 mmol) in DMF (110 mL) was added K₂CO₃ (9.32 g, 67.44 mmol) and 1,2-dibromoethane (21.12 g, 112.41 mmol, 8.5 mL), then the mixture was stirred at 25° C. for 12 h. The mixture was poured into water (200 mL) and extracted with EA (100 mL). The combined organic layers were dried over Na₂SO₄, filtered and concentrated under vacuum to give a residue. The residue was purified by column chromatography (PE/EA=10/1 to 1:1, SiO₂) and the elution was evaporated to give (4-bromo-2-chloro-6-(vinylthio)phenyl)methanol (2.7 g, 9.66 mmol, 43% yield) as colorless oil.

¹H NMR (400 MHz, DMSO-d₆) δ=7.65 (d, J=2.0 Hz, 1H), 7.42 (d, J=2.0 Hz, 1H), 8.78-6.71 (m, 1H), 5.81-5.52 (m, 2H), 5.25-5.23 (m, 1H), 4.62 (d, J=5.2 Hz, 2H) ppm

### Step 5: Preparation of methyl 3-chloro-4-(hydroxymethyl)-6-(vinylthio)benzoate

To a mixture (4-bromo-2-chloro-8-(vinylthio)phenyl)methanol (1000 mg, 3.58 mmol) in MeOH (20 mL) and TEA (10 mL) was added Pd(OAc)₂ (80.30 mg, 357.68 μmol) and XPhos (341 mg, 0.715 mmol), then the mixture was degassed and purged with CO (15 psi) for 3 times, and then the mixture was stirred at 70° C. for 8 h under CO (15 psi) atmosphere. The mixture was diluted with water (20 mL), was extracted with EA (15 mL×3), the combined organic phase was washed with brine (30 mL), dried over Na₂SO₄, filtered and concentrated under vacuum to give a crude product. The residue was purified by column chromatography (SiO₂, Petroleum ether/Ethyl acetate=20/1 to 5/1). The fraction was concentrated under vacuum to give methyl 3-chloro-4-(hydroxymethyl)-5-(vinylthio)benzoate (650 mg, 2.38 mmol, 88% yield) as a white solid.

¹H NMR (400 MHz, DMSO-d₆) δ=7.83 (d, J=1.6 Hz, 1H), 7.80 (d, J=1.6 Hz, 1H), 8.74-6.67 (m, 1H), 5.82-5.51 (m, 2H), 5.36-5.33 (m, 1H), 4.70 (d, J=5.2 Hz, 2H), 3.87 (s, 3H) ppm

### Step 6: Preparation of methyl 3-chloro-4-(hydroxymethyl)-5-(vinylsulfonyl)benzoate

To a mixture of methyl 3-chloro-4-(hydroxymethyl)-5-(vinylthio)benzoate (500 mg, 1.93 mmol) in H₂O (5 mL) and MeOH (5 mL) was added Oxone® (3.56 g, 5.80 mmol), the mixture was stirred at 25° C. for 1 hr. The mixture was diluted with water (200 mL), then extracted with EA (250 mL×2), the combined organic solution was washed with sat·Na₂SO₃ (150 mL×2) and brine (100 mL), dried over Na₂SO₄, filtered and concentrated under reduced pressure to give methyl 3-chloro-4-(hydroxymethyl)-5-vinylsulfonyl-benzoate (560 mg, crude) was obtained as a yellow oil LCMS (ESI) m/z: [M+H]⁺=273.0

¹H NMR (400 MHz, DMSO-d₆) δ=8.42 (d, J=1.6 Hz, 1H), 8.27 (d, J=1.6 Hz, 1H), 7.34-7.27 (m, 1H), 6.47-6.31 (m, 2H), 5.52-5.49 (m, 2H), 4.98 (d, J=5.2 Hz, 2H), 3.91 (s, 3H) ppm.

### Step 7: Preparation of 6-chloro-2,3-dihydro-6H-benzo[e][1,4]oxathiepine-8-carboxylic acid 1,1-dioxide (Intermediate 4)

To a mixture of methyl 3-chloro-4-(hydroxymethyl)-5-vinylsulfonyl-benzoate (560 mg, 1.93 mmol) in THE (18 mL) was added NaH (154.09 mg, 3.85 mmol, 60% purity) at 0° C., the mixture was stirred at 0° C. for 1 hr. The mixture was diluted with water (10 mL) and MeOH (5 mL), then stirred at 25° C. for 15 min. the resulting mixture was diluted with water (100 mL), acidized to pH 2 with HCl (1 N), the resulting solution was extracted with EA (150 mL×2). The combined organic layers were washed with brine (200 mL), dried over Na₂SO₄, filtered and concentrated under reduced pressure to give 6-chloro-2,3-dihydro-5H-benzo[e][1,4]oxathiepine-8-carboxylic acid 1,1-dioxide (300 mg, crude) was obtained as a white solid.

¹H NMR (400 MHz, DMSO-d₆) δ=13.91-13.84 (m, 1H), 8.38 (d, J=1.6 Hz, 1H), 8.22 (d, J=1.6 Hz, 1H), 5.19 (s, 2H), 4.23-4.21 (m, 2H), 3.79-3.77-3.74 (m, 2H) ppm.

### Intermediate 5: 6-Fluoro-2,3-dihydro-6H-benzo[e][1,4]oxathiepine-8-carboxylic acid 1,1-dioxide

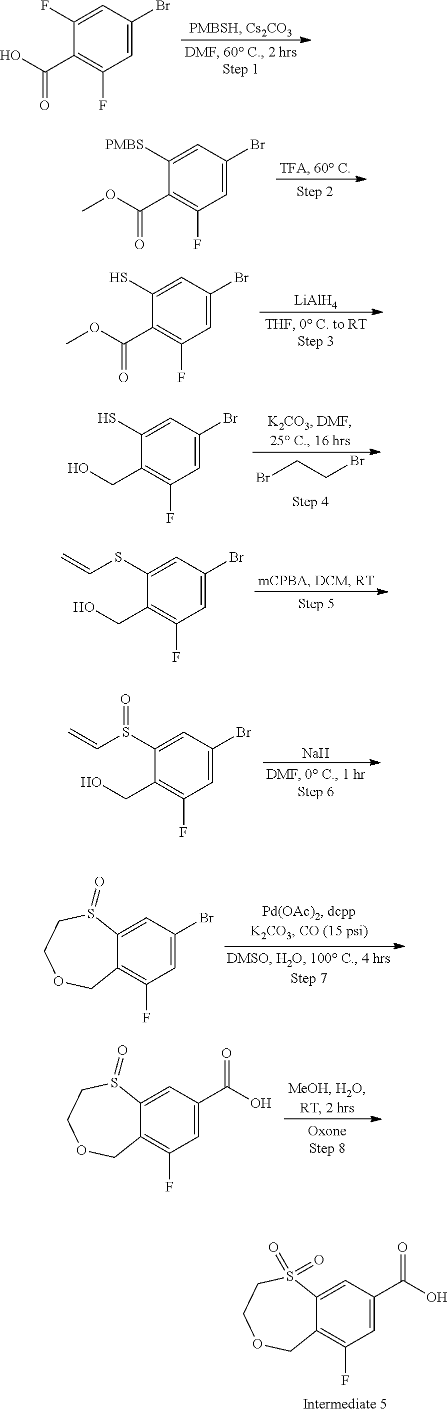

### Step 1: Preparation of methyl 4-bromo-2-fluoro-6-((4-methoxybenzyl)thio)benzoate

To a mixture of methyl 4-bromo-2,6-difluoro-benzoate (5 g, 19.92 mmol) and (4-methoxyphenyl)methanethiol (3.07 g, 19.92 mmol, 2.77 mL) in DMF (50 mL) was added Cs₂CO₃ (12.98 g, 39.84 mmol), then the mixture was stirred at 60° C. for 2 h. The mixture was diluted with water (400 mL), the extracted with EA (200 mL×3), the combined organic layers was washed with brine (200 mL×2), then dried over Na₂SO₄, filtered and concentrated under vacuum to give methyl 4-bromo-2-fluoro-6-((4-methoxybenzyl)thio)benzoate (9 g, crude) as yellow oil, which was used to next step directly.

¹H NMR (400 MHz, DMSO-d₆) δ=7.54-7.51 (m, 2H), 7.29-7.26 (m, 2H), 6.90-6.87 (m, 2H), 4.29 (s, 2H), 3.83 (s, 3H), 3.73-3.72 (m, 3H) ppm.

### Step 2: Preparation of methyl 4-bromo-2-fluoro-6-mercaptobenzoate

A mixture of methyl 4-bromo-2-fluoro-6-((4-methoxybenzyl)thio)benzoate (9 g, 23.36 mmol) in TFA (138.60 g, 1.22 mol, 90 mL) was stirred at 60° C. for 2 h. The mixture was evaporated and then neutralized with by sat·NaHCO₃ to pH 7. Then the mixture was extracted with EA (200 mL). The organic layer was separated and dried over anhydrous Na₂SO₄. The organic phase was concentrated under vacuum to give methyl 4-bromo-2-fluoro-6-mercaptobenzoate (6 g, crude) as yellow oil, which was used to next step directly.

¹H NMR (400 MHz, DMSO-d₆) δ=7.77-7.61 (m, 2H), 3.93 (s, 3H) ppm.

### Step 3: Preparation of (4-bromo-2-fluoro-6-mercaptophenyl)methanol

To a mixture of methyl 4-bromo-2-fluoro-6-mercaptobenzoate (3.4 g, 12.83 mmol) in THE (34 mL) was added LiAlH₄ (535.5 mg, 14.11 mmol), then the mixture was stirred at 0° C. for 1 hr. The mixture was quenched with 1N HCl (100 mL) and extracted with EA (50 mL). The organic layer was separated and dried over anhydrous Na₂SO₄, filtered and concentrated under vacuum to give (4-bromo-2-fluoro-6-mercaptophenyl)methanol (3 g, crude) as a yellow oil, which was used to next step directly.

¹H NMR (400 MHz, DMSO-d₆) δ=7.51 (s, 1H), 7.32-7.24 (m, 1H), 4.45 (d, J=1.2 Hz, 2H)

### Step 4: Preparation of (4-bromo-2-fluoro-6-(vinylthio)phenyl)methanol

To a mixture of (4-bromo-2-fluoro-6-mercaptophenyl)methanol (3 g, 12.85 mmol), K₂CO₃ (5.25 g, 37.96 mmol) In DMF (60 mL) was added 1,2-dibromoethane (11.89 g, 63.27 mmol), then the mixture was stirred at 25° C. for 15 h. The mixture was poured into water (200 mL) and extracted with EA (100 mL). The combined organic layer was dried over Na₂SO₄, filtered, and concentrated under vacuum to give a residue. The residue was purified by column chromatography (PE/EA=10/1, SiO₂) and the eluent was evaporated to give the (4-bromo-2-fluoro-6-(vinylthio)phenyl)methanol (1.6 g, 6.08 mmol, 48% yield) as colorless oil.

¹H NMR (400 MHz, DMSO-d₆) δ=7.47-7.44 (m, 1H), 7.30-7.29 (m, 1H), 6.77-6.70 (m, 1H), 5.59-5.52 (m, 2H), 5.22-5.20 (m, 1H), 4.51-4.48 (m, 2H) ppm

### Step 6: Preparation of (4-bromo-2-fluoro-6-(vinylsulfinyl)phenyl)methanol

To a mixture of (4-bromo-2-fluoro-6-(vinylthio)phenyl)methanol (800 mg, 3.04 mmol) in DCM (12 mL) was added m-CPBA (678.98 mg, 3.34 mmol, 85% purity) at 0° C., then the mixture was stirred at 25° C. for 1 hr. The reaction mixture was quenched by addition saturated aqueous Na₂SO₃ (20 mL) at 0° C., and then diluted with H₂O (20 mL) and extracted with EA (100 mL×3). The combined organic layers were washed with brine 100 ml, dried over Na₂SO₄, filtered, and concentrated under reduced pressure to give a residue. The residue was purified by column chromatography (SiO₂, Petroleum ether/Ethyl acetate=20/1 to 1/1). The fraction was concentrated under vacuum to give (4-bromo-2-fluoro-6-(vinylsulfinyl)phenyl)methanol (690 mg, 2.47 mmol, 81% yield) as a yellow solid, LCMS (ESI) m/z: [⁷⁹BrM+H]⁺=278.9

¹H NMR (400 MHz, DMSO-d₆) δ=7.74-7.71 (m, 1H), 7.61-7.60 (m, 1H), 7.12-7.06 (m, 1H), 6.05-5.92 (m, 2H), 5.85-5.82 (m, 1H), 4.75-4.71 (m, 1H), 4.63-4.58 (m, 1H) ppm

### Step 6: Preparation of 8-bromo-6-fluoro-2,3-dihydro-6H-benzo[e][1,4]oxathiepine 1-oxide

To a mixture of (4-bromo-2-fluoro-6-(vinylsulfinyl)phenyl)methanol (650 mg, 2.33 mmol) in DMF (40 mL) was added NaH (186.3 mg, 4.66 mmol, 60% purity) at 0° C. then the mixture was stirred at 0° C. for 1 hr. The reaction solution was quenched with saturated aqueous NH₄Cl 50 mL and extracted with EA (50 mL×3). The combined organic layers were washed with brine (60 mL×3), dried over Na₂SO₄, filtered, and concentrated under reduced pressure to give a residue. The residue was purified by column chromatography (SiO₂, Petroleum ether/Ethyl acetate=10/1 to 1/1) and the eluent was concentrated under reduced pressure to give 8-bromo-6-fluoro-2,3-dihydro-5H-benzo[e][1,4]oxathiepine 1-oxide (350 mg, 1.25 mmol, 54% yield) as a white solid.

¹H NMR (400 MHz, CDCl₃) δ=7.79 (m, 1H), 7.38-7.35 (m, 1H), 5.13 (d, J=14.4 Hz, 1H), 4.49-4.34 (m, 3H), 3.47-3.41 (m, 1H), 3.26-3.21 (m, 1H) ppm.

### Step 7: Preparation of 6-fluoro-2,3-dihydro-6H-benzo[e][1,4]oxathiepine-8-carboxylic acid 1-oxide

To a mixture of 8-bromo-6-fluoro-2,3-dihydro-5H-benzo[e][1,4]oxathiepine 1-oxide (320 mg, 1.15 mmol), Pd(OAc)₂ (12.87 mg, 57.32 μmol) and dicyclohexyl(3-dicyciohexylphosphaniumylpropyl)phosphonium;ditetrafluoroborate (70.19 mg, 114.64 μmol) in DMSO (4 mL) and H₂O (0.2 mL) was added K₂CO₃ (475.33 mg, 3.44 mmol), then the mixture was stirred at 100° C. for 4 h under CO (15 psi). The mixture was diluted with water (50 mL) and extracted with EA (30 mL×3). The aqueous layer was acidized to pH=3 by HCl solution (2 M) and extracted with EA (100 mL×2). The combined organic phase was washed with brine, dried over Na₂SO₄, filtered and concentrated under vacuum to give 6-fluoro-2,3-dihydro-5H-benzo[e][1,4]oxathiepine-8-carboxylic acid 1-oxide (210 mg, crude) as a white solid, which was used to next step directly.

LCMS (ESI) m/z: [M+H]⁺=244.9

### Step 8: Preparation of 6-fluoro-2,3-dihydro-6H-benzo[e][1,4]oxathiepine-8-carboxylic acid 1,1-dioxide (Intermediate 5)

To a mixture of 6-fluoro-2,3-dihydro-5H-benzo[e][1,4]oxathiepine-8-carboxylic acid 1-oxide (210.00 mg, 859.81 μmol) in MeOH (4 mL) and H₂O (4 mL) was added Oxone® (634.30 mg, 1.03 mmol), then the mixture was stirred at 25° C. for 2 h. The mixture was diluted with water (50 mL), then extracted with EA (30 mL×3), the combined organic layers was washed with brine (40 mL×2), dried over Na₂SO₄, filtered and concentrated under vacuum to give 6-fluoro-2,3-dihydro-5H-benzo[e][1,4]oxathiepine-8-carboxylic acid 1,1-dioxide (220 mg, crude) as a white solid, which was used to next step directly.

### Intermediates 6 and 7. (R)-2-Methyl-2,3-dihydro-5H-benzo[e][1,4]oxathiepine-8-carboxylic acid 1,1-dioxide and (S)-2-methyl-2,3-dihydro-5H-benzo[e][1,4]oxathiepine-8-carboxylic acid 1,1-dioxide

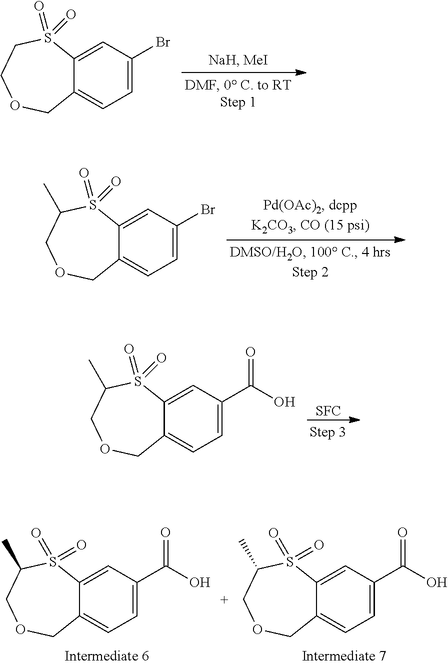

### Step 1: Preparation of 8-bromo-2-methyl-3,5-dihydro-2H-benzo[e][1,4]oxathiepine 1,1-dioxide

To a solution of 8-bromo-2,3-dihydro-5H-benzo[e][1,4]oxathiepine 1,1-dioxide (380 mg, 1.37 mmol, 641.03 uL) in DMF (5 mL) was added NaH (65.82 mg, 1.65 mmol, 60% purity) at 0° C. The mixture was stirred at 0° C. for 0.5 h. Then MeI (233.55 mg, 1.65 mmol, 102.43 uL) was added slowly at 0° C. The mixture was stirred at 25° C. for 1.5 h. The mixture was diluted with saturated NH₄Cl solution (30 mL) and extracted with EtOAc (30 mL×2). The combined organic layer was dried with anhydrous Na₂SO₄ and concentrated to afford residue. The residue was purified by reversed-phase HPLC (0.1% FA condition). The eluent was concentrated to remove MeCN and lyophilized to afford 8-bromo-2-methyl-3,5-dihydro-2H-benzo[e][1,4]oxathiepine 1,1-dioxide (100 mg, 309.11 μmol, 23% yield) as a white solid.

LCMS (ESI) m/z: [M+H]⁺=291.0/292.9

¹HNMR (400 MHz, DMSO-d₆) δ=7.99 (d, J=2.0 Hz, 1H), 7.95-7.92 (m, 1H), 7.56 (d, J=8.0 Hz, 1H), 4.87 (s, 2H), 4.27-4.23 (m, 1H), 4.00-3.95 (m, 1H), 3.71-3.62 (m, 1H), 1.14 (d, J=7.2 Hz, 3H) ppm.

### Step 2: Preparation of 2-methyl-3,5-dihydro-2H-benzo[e][1,4]oxathiepine-8-carboxylic acid 1,1-dioxide

To a solution of 8-bromo-2-methyl-3,5-dihydro-2H-benzo[e][1,4]oxathiepine 1,1-dioxide (100 mg, 343.45 μmol) in DMSO (1 mL) and H₂O (30.95 mg, 1.72 mmol, 31 μL) was added 1,3-bis(dicyclohexylphosphino)propane bis(tetrafluoroborate) (21.03 mg, 34.35 μmol), K₂CO₃ (71.20 mg, 515.18 μmol) and Pd(OAc)₂ (7.71 mg, 34.35 μmol). The flask was degassed and purged with CO for 3 times. The mixture was stirred at 100° C. for 4 h under CO (15 psi) atmosphere. The mixture was filtered and washed by EA (2 mL and water (2 mL). Then the mixture was diluted with water (5 mL) and extracted with EA (5 mL×2). The combined organic layer was discarded. The aqueous phase was adjusted pH=6 with 1N aq. HCl. Then the aqueous phase was extracted with EA (5 mL×2). The combined organic layer was washed by brine (5 mL×2), dried with anhydrous Na₂SO₄ and concentrated to afford 2-methyl-3,5-dihydro-2H-benzo[e][1,4]oxathiepine-8-carboxylic acid 1,1-dioxide (80 mg, 0.290 mol, 85% yield) as a white solid.

LCMS (ESI) m/z: [M+H]⁺=258.9.

¹HNMR (400 MHz, DMSO-d₆) δ=8.44 (d, J=1.6 Hz, 1H), 8.22-8.19 (m, 1H), 7.72 (d, J=8.0 Hz, 1H), 4.95 (s, 2H), 4.29-4.25 (m, 1H), 4.03-3.98 (m, 1H), 3.72-3.60 (m, 1H), 1.14 (d, J=6.8 Hz, 3H) ppm.

### Step 3: Preparation of (R)-2-methyl-3,5-dihydro-2H-benzo[e][1,4]oxathiepine-8-carboxylic acid 1,1-dioxide (Intermediate 6) and (S)-2-methyl-3,5-dihydro-2H-benzo[e][1,4]oxathiepine-8-carboxylic acid 1,1-dioxide (intermediate 7)

Racemic 2-methyl-3,5-dihydro-2H-benzo[e][1,4]oxathiepine-8-carboxylic acid 1,1-dioxide was separated by SFC (column: Daicel ChiralPak IG (250×30 mm, 10 um); mobile phase: [0.1% NH₃H₂O MEOH];B %:30%-30%, 3.0;85 min). The eluent was concentrated to remove most of the solvent and adjusted to pH=6 with FA. Then the mixture was extracted with DCM (20 mL×2). The combined organic layer was dried with anhydrous Na₂SO₄ and concentrated to afford (R)-2-methyl-3,5-dihydro-2H-benzo[e][1.4]oxathiepine-8-carboxylic acid 1,1 dioxide (35 mg, 0.138 mmol, 44% yield) as a white solid and (S)-2-methyl-3,5-dihydro-2H-benzo[e][1,4]oxathiepine-8-carboxylic acid 1,1-dioxide (40 mg, 0.158 mmol, 50.00% yield) as a white solid. Stereochemistry was assigned arbitrarily.

### (R)-2-methyl-3,5-dihydro-2H-benzo[e][1,4]oxathiepine-8-carboxylic acid 1,1-dioxide (Intermediate 6)

LCMS (ESI) m/z: [M+Na]₌279.1.

Chiral SFC: IG-3_5CM_MEOH(DEA)_5_40_3ML T35.M; Rt=1.729 mins.

### (S)-2-methyl-3,5-dihydro-2H-benzo[e][1,4]oxathiepine-8-carboxylic acid 1,1-dioxide (Intermediate 7)

LCMS (ESI) m/z: [M+Na]⁺=279.1.

Chiral SFC: IG-3_5CM_MEOH(DEA)_5_40_3ML_T35.M; Rt=1.897 mins.

### Intermediate 8. 6,7,8,9-Tetrahydrothiepino[3,2-b]pyridine-3-carboxylic acid 5,5-dioxide

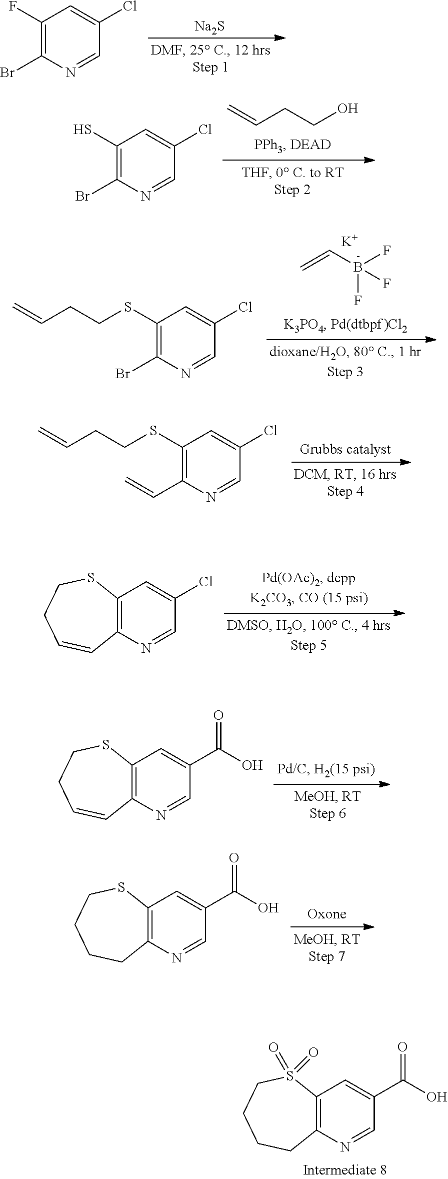

### Step 1: Preparation of 2-bromo-5-chloro-pyridine-3-thiol

To a mixture of 2-bromo-5-chloro-3-fluoro-pyridine (1.3 g, 6.18 mmol, 1 eq) in DMF (20 mL) was added Na₂S (482.14 mg, 6.18 mmol, 259.22 μL) in one portion at 25° C. under N₂. The mixture was stirred at 25° C. for 12 h. The mixture was poured into (100 mL). To the mixture was added aqueous HCl (2M) to adjust pH=3. The aqueous phase was extracted with ethyl acetate (50 mL×2). The combined organic phase was washed with brine (50 mL×1), dried with anhydrous Na₂SO₄, filtered and concentrated under vacuum to afford 2-bromo-5-chloro-pyridine-3-thiol (1.2 g, 5.35 mmol, 86.52% yield) as yellow solid.

LCMS (ESI) m/z: (⁷⁹BrM+H]⁺=225.8.

¹H NMR (400 MHz, DMSO-d₆) δ=8.33-8.23 (m, 1H), 8.17-8.08 (m, 1H) ppm.

### Step 2: Preparation of 2-bromo-3-(but-3-en-1-ylthio)-5-chloropyridine

To a mixture of 2-bromo-5-chloro-pyridine-3-thiol (1.2 g, 5.35 mmol) and but-3-en-1-ol (385.41 mg, 5.35 mmol, 459.91 μL) in THE (10 mL) was added PPh₃ (2.10 g, 8.02 mmol) followed by DEAD (1.40 g, 8.02 mmol, 1.46 mL) dropwise at 0° C. under N₂. The mixture was stirred at 25° C. for 12 h. The mixture was poured into water (50 mL) and extracted with ethyl acetate (30 mL×2). The combined organic phase was washed with brine (30 mL×1), dried with anhydrous Na₂SO₄, filtered and concentrated under vacuum to afford a residue. The residue was purified by silica gel chromatography (Petroleum ether/Ethyl acetate=10/1). The eluent was concentrated to afford 2-bromo-3-(but-3-en-1-ylthio)-5-chloropyridine (1.2 g, 4.31 mmol, 81% yield) as yellow oil.

LCMS (ESI) m/z: [⁷⁹BrM+H]⁺=277.9.

¹H NMR (400 MHz, CDCl₃) δ=8.05-7.99 (m, 1H), 7.34-7.28 (m, 1H), 5.88-5.73 (m, 1H), 5.16-5.01 (m, 2H), 2.99-2.88 (m, 2H), 2.48-2.35 (m, 2H) ppm.

### Step 3: Preparation of 3-(but-3-en-1-ylthio)-5-chloro-2-vinylpyridine

A mixture of 2-bromo-3-(but-3-en-1-ylthio)-5-chloropyridine (880 mg, 3.09 mmol), potassium vinyltrifluoroborate (1.24 g, 9.26 mmol), Pd(dtbpf)Cl₂ (201.19 mg, 308.69 μmol) and K₃PO₄ (1.97 g, 9.26 mmol) in dioxane (12 mL) and H₂O (3 mL) was stirred at 80° C. for 1 hr under N₂. The mixture was poured into H₂O (100 mL) and extracted with EA (30 mL×3). The combined organic layers were washed with brine (20 mL×2), dried over anhydrous Na₂SO₄, filtered and concentrated under reduced pressure to afford a residue. The residue was purified by silica gel chromatography (PE-PE/EA=20/1). The eluent was concentrated under reduced pressure to afford 3-(but-3-en-1-ylthio)-5-chloro-2-vinylpyridine (510 mg, 2.26 mmol, 73.% yield) as yellow oil.

LCMS (ESI) m/z: [M+H]⁺=226.0.

¹H NMR (400 MHz, CDCl₃) δ=8.36 (d, J=2.4 Hz, 1H), 7.60 (d, J=2.4 Hz, 1H), 7.26-7.19 (m, 1H), 6.41-6.36 (m, 1H), 5.89-5.81 (m, 1H), 5.57-5.53 (m, 1H), 5.20-5.07 (m, 2H), 2.97-2.93 (m, 2H), 2.44-2.36 (m, 2H) ppm.

### Step 4: Preparation of 3-chloro-6,7-dihydrothiepino[3,2-b]pyridine

A mixture of 3-(but-3-en-1-ylthio)-5-chloro-2-vinylpyridine (250 mg, 1.11 mmol) and benzylidene-[1,3-bis(2,4,6-trimethylphenyl)imidazolidin-2-ylidene]-dichloro-ruthenium;tricyclohexylphosphane (Grubbs II) (94.0 mg, 0.111 mol) in DCM (12 mL) was stirred at 25° C. for 16 h under N₂. The solution was concentrated under vacuum. The residue was purified by silica gel chromatography (PE-PE/EA=20/1). The eluent was concentrated under reduced pressure to afford 3-chloro-6,7-dihydrothiepino[3,2-b]pyridine (110 mg, 556.44 μmol, 50% yield) as a yellow oil.

LCMS (ESI) m/z: [M+H]⁺=198.0.

¹H NMR (400 MHz, CDCl₃) δ=8.40 (d, J=2.0 Hz, 1H), 7.71 (d, J=1.6 Hz, 1H), 6.81-6.71 (m, 1H), 6.32-6.26 (m, 1H), 3.10-3.05 (m, 2H), 2.88-2.81 (m, 2H)

### Step 5: Preparation of 6,7-dihydrothiepino[3,2-b]pyridine-3-carboxylic acid

A mixture of 3-chloro-6,7-dihydrothiepino[3,2-b]pyridine (50 mg, 0.253 mol), K₂CO₃ (52.44 mg, 0.379 mol), Pd(OAc)₂ (2.84 mg, 12.65 μmol), 1,3-bis(dicyclohexylphosphino)propane bis(tetrafluoroborate) (15.49 mg, 25.29 μmol) and H₂O (100 μL) in DMSO (1 mL) was stirred at 100° C. for 4 h under CO (15 psi). The mixture was poured into H₂O (10 mL) and extracted with EA (10 mL×2). The organic phase was discarded. The aqueous phase was acidified with HCl (1 M) to pH=3 and extracted with EA (10 mL×3). The combined organic layers were washed with brine (10 mL×2), dried over anhydrous Na₂SO₄, filtered and concentrated under reduced pressure to afford 6,7-dihydrothiepino[3,2-b]pyridine-3-carboxylic acid (28 mg, 135.10 μmol, 53.4% yield) as white solid.

LCMS (ESI) m/z: [M+H]⁺=207.9.

¹H NMR (400 MHz, CDCl₃) δ=8.36 (d, J=2.4 Hz, 1H), 7.60 (d, J=2.4 Hz, 1H), 7.26-7.19 (m, 1H), 6.41-6.36 (m, 1H), 5.89-5.81 (m, 1H), 5.57-5.53 (m, 1H), 5.20-5.07 (m, 2H), 2.97-2.93 (m, 2H), 2.44-2.38 (m, 2H) ppm.

### Step 6: Preparation of 6,7,8,9-tetrahydrothiepino[3,2-b]pyridine-3-carboxylic acid

To mixture of 6,7-dihydrothiepino[3,2-b]pyridine-3-carboxylic acid (28 mg, 135.10 μmol) in MeOH (5 mL) was added Pd/C (wet, 50 mg, 10% purity) at 25° C. The mixture was purged with H₂ for 3 times and stirred at 25° C. for 30 min under H₂ (15 psi). The mixture was filtered and the filtrate was concentrated under reduced pressure to afford 6,7,8,9-tetrahydrothiepino[3,2-b]pyridine-3-carboxylic acid (23 mg, 109.91 μmol, 81.2% yield) as white solid.

LCMS (ESI) m/z: [M+H]⁺=210.0.

¹H NMR (400 MHz, DMSO-d₆) δ=8.81 (d, J=2.0 Hz, 1H), 8.20 (d, J=2.0 Hz, 1H), 3.23-3.17 (m, 2H), 2.88-2.78 (m, 2H), 2.11-1.96 (m, 2H), 1.76-1.62 (m, 2H) ppm.

### Step 7: Preparation of 5,5-dioxo-6,7,8,9-tetrahydrothiepino[3,2-b]pyridine-3-carboxylic acid (Intermediate 8)

To a mixture of 6,7,8,9-tetrahydrothiepino[3,2-b]pyridine-3-carboxylic acid (23 mg, 109.91 μmol) in MeOH (1 mL) and H₂O (1 mL) was added Oxone® (67.57 mg, 109.9 μmol) at 25° C. The mixture was stirred at 25° C. for 4 h. The mixture was quenched with sat. Na₂SO₃ (20 mL), acidified with HCl (1 M) to pH=2 and extracted with EA (20 mL×2). The combined organic layers were washed with brine dried over anhydrous Na₂SO₄, filtered and concentrated under reduced pressure to afford 5,5-dioxo-6,7,8,9-tetrahydrothiepino[3,2-b]pyridine-3-carboxylic acid (18 mg, 74.61 μmol, 88% yield) as white solid.

LCMS (ESI) m/z: [M+H]⁺=241.9.

¹H NMR (400 MHz, DMSO-d₆) δ=9.14 (d, J=2.0 Hz, 1H), 8.55 (d, J=2.1 Hz, 1H), 3.53-3.51 (m, 2H), 3.17 (br d, J=5.2 Hz, 2H), 2.19-2.13 (m, 2H), 1.82 (br d. J=3.2 Hz, 2H) ppm.

### Intermediate 9: 4,5-dihydro-2H-benzo[d][1,3]oxathiepine-8-carboxylic acid 1,1-dioxide

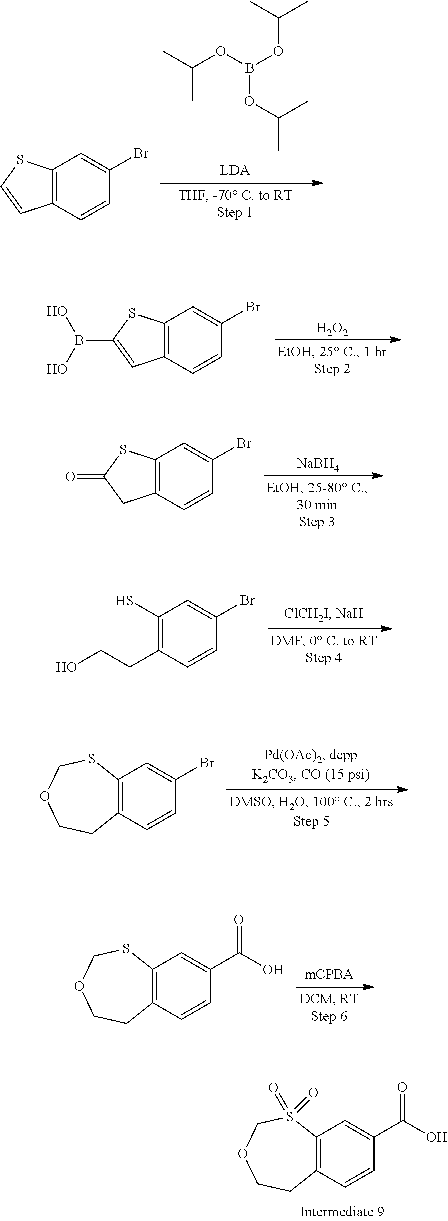

### Step 1: Preparation of (6-bromobenzo[b]thiophen-2-yl)boronic acid

To a mixture of 8-bromobenzo[b]thiophene (8 g, 37.54 mmol) in THE (80 mL) was added LDA (2 M, 22.53 mL) dropwise at −70° C. under N₂. The mixture was stirred at −70° C. for 1 hr. Then to the mixture was added triisopropyl borate (8.47 g, 45.05 mmol, 10.36 mL) at −70° C., and the mixture was stirred for 1 hr. To the mixture was added H₂SO₄ (7.36 g, 75.08 mmol, 4.00 mL) at −70° C., and the mixture was stirred at 25° C. for 1 hr. The mixture was poured into water (300 mL) and extracted with ethyl acetate (200 ml×2). The combined organic phase was washed with brine (200 mL×1), dried with anhydrous Na₂SO₄, filtered and concentrated in vacuum. The residue was triturated by PE/MTBE=10/1 (50 mL). The suspension was filtered. The filter cake was dried under pump to afford (6 bromobenzo[b]thiophen-2-yl)boronic acid (7.3 g, 28.41 mmol, 78% yield) as light yellow solid.

¹H NMR (400 MHz, DMSO-d6) δ=8.58-8.53 (m, 2H), 8.28-8.24 (m, 1H), 7.96-7.93 (m, 1H), 7.88-7.84 (m, 1H), 7.54-7.48 (m, 1H) ppm.

### Step 2: Preparation of 6-bromobenzo[b]thiophen-2(3H)-one

To a mixture of (6-bromobenzo[b]thiophen-2-yl)boronic acid (6.5 g, 25.30 mmol) in EtOH (78 mL) was added H₂O₂ (38.35 g, 338.24 mmol, 32.50 mL) dropwise at 25° C. under N₂. The mixture was stirred at 25° C. for 1 hr. The mixture was filtered. The filter cake was washed with H₂O (50 mL) and dried in vacuum to afford 6-bromobenzo[b]thiophen-2(3H)-one (4.2 g, 18.33 mmol, 72% yield) as brown solid.

LCMS (ESI) m/z: [M+H]⁺=214.8, 216.9.

¹H NMR (400 MHz, CDCl₃) δ=7.53-7.47 (m, 1H), 7.38-7.32 (m, 1H), 7.18 (d, J=8.0 Hz, 1H), 4.06-3.84 (m, 2H) ppm.

### Step 3: Preparation of 2-(4-bromo-2-mercaptophenyl)ethan-1-oi

To a mixture of 6-bromobenzo[b]thiophen-2(3H)-one (4.2 g, 18.33 mmol) in EtOH (67 mL) was added NaBH₄ (3.47 g, 91.67 mmol) in portions at 25° C. under N₂. The mixture was stirred at 80° C. for 30 min. The mixture was cooled to 25° C. To the mixture was added aqueous HCl (1 M) slowly to adjust pH=2. The mixture was poured into water (200 mL) and extracted with ethyl acetate (100 mL×2). The combined organic phase was washed with brine (100 mL), dried over anhydrous Na₂SO₄, filtered, and concentrated in vacuum. The residue was purified by silica gel chromatography (Petroleum ether/Ethyl acetate=2/1). The eluent was concentrated to afford 2-(4-bromo-2-mercaptophenyl)ethan-1-ol (3.6 g, 15.44 mmol, 84% yield) as yellow oil.

¹H NMR (400 MHz, DMSO-d8) δ=7.81 (d, J=2.0 Hz, 1H), 7.24-7.22 (m, 1H), 7.13 (d, J=8.2 Hz, 1H), 5.58 (s, 1H), 4.97-4.49 (m, 1H), 3.59-3.57 (m, 2H), 2.71-2.69 (m, 2H).

### Step 4: Preparation 8-bromo-4,5-dihydrobenzo[d][1,3]oxathiepine

To a mixture of 2-(4-bromo-2-mercaptophenyl)ethan-1-ol (500 mg, 2.14 mmol) in DMF (50 mL) was added NaH (257.37 mg, 6.43 mmol) in portions at 0° C. under N₂. The mixture was stirred at 25° C. for 30 min. Then to the mixture was added chloro(iodo)methane (416.13 mg, 2.38 mmol, 171 μL) in DMF (1 mL) dropwise at 0° C. under N₂. The mixture was stirred at 25° C. for 1.5 h. The mixture was poured into sat·NH₄Cl (10 mL) and extracted with ethyl acetate (10 mL×2). The combined organic phase was washed with brine (10 mL), dried over anhydrous Na₂SO₄, filtered and concentrated in vacuum. The residue was purified by silica gel chromatography (Petroleum ether/Ethyl acetate=10/1). The eluent was concentrated to afford 8-bromo-4,5-dihydrobenzo[d][1,3]oxathiepine (50 mg, 0.189 mmol, 9% yield) as yellow oil.

LCMS (ESI) m/z: [M+H]⁺=246.2, 248.0.

¹H NMR (400 MHz, DMSO-d6) δ=7.67-7.60 (m, 1H), 7.47-7.38 (m, 1H), 7.31-7.22 (m, 1H), 5.00-4.86 (m, 2H), 3.81-3.67 (m, 2H), 3.12-3.09 (m, 2H) ppm.

### Step 5: Preparation of 4,5-dihydrobenzo[d][1,3]oxathiepine-8-carboxylic acid

A solution of 8-bromo-4,5-dihydrobenzo[d][1,3]oxathiepine (50 mg, 203.97 μmol), Pd(OAc)₂ (4.58 mg, 20.40 μmol), 1,3-bis(dicyclohexylphosphino)propane bis(tetrafluoroborate) (24.98 mg, 40.79 μmol) and K₂CO₃ (56.38 mg, 0.408 mmol) in DMSO (2 mL) and H₂O (0.2 mL) was degassed under vacuum and purged with CO several times. The mixture was stirred under CO (15 psi) at 100° C. for 2 h. The mixture was poured into water (20 mL) and extracted with ethyl acetate (10 mL×2). The organic layer was discarded. To the aqueous phase was added aqueous HCl (1 M) to adjust pH=3. The mixture was extracted with ethyl acetate (10 mL×2). The combined organic phase was washed with brine (10 mL), dried over anhydrous Na₂SO₄, filtered and concentrated in vacuum to afford 4,5-dihydrobenzo[d][1,3]oxathiepine-8-carboxylic acid (40 mg, 190.25 μmol, 93% yield) as yellow solid LCMS (ESI) m/z: [M+H]⁺=211.1.

### Step 6: Preparation of 4,5-dihydro-2H-benzo[d][1,3]oxathiepine-8-carboxylic acid 1,1-dioxide (Intermediate 9)

To a mixture of 4,5-dihydrobenzo[d][1,3]oxathiepine-8-carboxylic acid (20 mg, 95.13 μmol) in DCM (1 mL) was added mCPBA (48.28 mg, 237.81 μmol, 85% purity) in portions at 25° C. under N₂. The mixture was stirred at 25° C. for 12 h. The mixture was filtered and the filtrate was concentrated. The residue was purified by reverse phase column (FA) directly. The eluent was concentrated to remove MeCN. The aqueous phase was lyophilized to afford 4,5-dihydrobenzo[d][1,3]oxathiepine-8-carboxylic acid 1,1-dioxide (20 mg, 82.56 μmol, 86.79% yield) as a white solid.

LCMS (ESI) m/z: [M+H₂O]⁺=260.0.

¹H NMR (400 MHz, DMSO-d8) δ=8.38 (d, J=1.6 Hz, 1H), 8.16-8.14 (m, 1H), 7.62 (d, J=7.8 Hz, 1H), 4.99 (s, 2H), 4.01-4.00 (m, 2H), 3.42 (s, 2H) ppm.

### Intermediate 10: 4,5-dihydro-2H-benzo[d][1,3]oxathiepine-8-carboxylic acid 1,1-dioxide

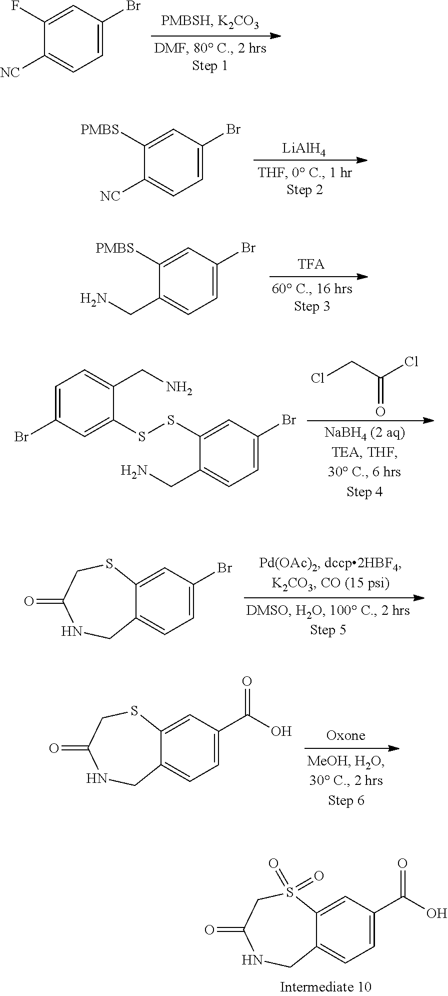

### Step 1: Preparation of 4-bromo-2-[(4-methoxyphenyl)methylsulfanyl]benzonitrile

To a solution of 4-bromo-2-fluoro-benzonitrile (10 g, 50.00 mmol) and (4-methoxyphenyl)methanethiol (7.71 g, 50.00 mmol) in DMF (100 mL) was added Cs₂CO₃ (16.29 g, 50.00 mmol), the mixture was stirred at 60° C. 2 h. The reaction mixture was poured into water (1000 mL), the solution was extracted with EA (1000 mL×3), the combined organic layer was washed with brine (500 mL), dried over Na₂SO₄, filtered and concentrated to give 4-bromo-2-[(4-methoxyphenyl)methylsulfanyl]benzonitrile (13 g, crude) as a white solid.

### Step 2: Preparation of [4-bromo-2-[(4-methoxyphenyl)methylsulfanyl]phenyl]methanamine

To a solution of 4-bromo-2-[(4-methoxyphenyl)methylsulfanyl]benzonitrile (13 g, 38.90 mmol) in THE (150 mL) was added LiAlH₄ (1.62 g, 42.78 mmol) at 0° C. under N₂, the mixture was stirred at 0° C. for 1 hr. To the mixture was poured into water (1.62 g) and 15% NaOH solution (2.5 mL), the solution was poured into EA (500 mL), the solution was filtered and the filtrate was concentrated to give [4-bromo-2-[(4-methoxyphenyl)methylsulfanylphenyl]methanamine (13 g. crude) as yellow oil.

1H NMR (400 MHz, DMSO-d₆) δ=7.48-7.47 (m, 1H), 7.21-7.20 (m, 1H), 7.19-7.18 (m, 3H), 6.85-6.82 (m, 2H), 4.08 (s, 2H), 3.80-3.79 (m, 5H) ppm

### Step 3: Preparation of [2-[2-(aminomethyl)-6-bromo-phenyl]disulfanyl]-4-bromo-phenyl]methanamine

A mixture of [4-bromo-2-[(4-methoxyphenyl)methylsulfanyl]phenyl]methanamine (13 g, 38.43 mmol) in TFA (130 mL) was stirred at 60° C. for 16 h. The reaction mixture was concentrated to give a residue. The residue was purified by reversed-phase HPLC (0.1% FA condition). The solution was lyophilizated to give aminomethyl)-5-bromo-phenyl]disulfanyl]-4-bromo-phenyl]methanamine (3.5 g, 7.20 mmol, 19% yield) as a white solid.

LCMS (ESI) m/z: [⁷⁹BrM+H]⁺=434.8

1H NMR (400 MHz, DMSO-d₆) δ=8.35 (br s, 3H), 7.55 (s, 2H), 7.50-7.37 (m, 1H), 4.05 (s, 2H) ppm

### Step 4: Preparation of 8-bromo-4,5-dihydro-1,4-benzothiazepin-3-one

To a solution of aminomethyl)-5-bromo-phenyl]disulfanyl]-4-bromo-phenyl]methanamine (1 g, 2.30 mmol) in THE (15 mL) was added NaBH₄ (261.37 mg, 6.91 mmol), the mixture was stirred at 30° C. for 2 h. Then to the solution was added TEA (11.52 mmol, 1.60 mL), 2-chioroacetyl chloride (312.13 mg, 2.78 mmol), the mixture was stirred at 30° C. for 3 h. The reaction mixture was poured into water (100 mL) and extracted with EA (100 mL×3). The combined organic layer was washed with brine (200 mL), dried over Na₂SO₄, filtered and concentrated to give a residue. The residue was purified by column chromatography (SiO₂, Petroleum ether/Ethyl acetate=10:1-0:1), the solution was concentrated to give 8-bromo-4,5-dihydro-1,4-benzothiazepin-3-one (300 mg, 871.29 μmol, 38% yield) as a white solid.

LCMS (ESI) m/z: [⁷⁹BrM+H]⁺=260.0

1H NMR (400 MHz, DMSO-d₆) δ=7.37 (d, J=2.0 Hz, 1H), 7.24-7.22 (m, 1H), 7.07 (d, J=8.0 Hz, 1H), 4.45 (s, 2H), 3.89 (s, 2H) ppm

### Step 5: Preparation of 3-oxo-4,5-dihydro-1,4-benzothiazepine-8-carboxylic acid (Intermediate 10)

To a solution of 8-bromo-4,5-dihydro-1,4-benzothiazepin-3-one (280 mg, 1.08 mmol) in DMSO (5 mL) was added dicyclohexyl(3-dicyciohexylphosphaniumylpropyl)phosphonium:ditetrafluoroborate (66.41 mg, 108.47 μmol), K₂CO₃ (224.88 mg, 1.63 mmol), Pd(OAc)₂ (24.35 mg, 108.47 μmol) and H₂O (3.91 mg, 216.94 μmol), the mixture was stirred under CO (15 psi) at 100° C. for 2 h. The reaction mixture was filtered, the solution was extracted with MTBE (10 mL), the organic layer was discarded. Then the aqueous phase was adjusted to pH=2 with 1 N HCl, the solution was extracted with EA (50 mL×5), the combined organic layer was washed with trine (100 mL), dried over Na₂SO₄, filtered and concentrated to give 3-oxo-4,5-dihydro-1,4-benzothiazepine-8-carboxylic acid (120 mg, 0.487 mmol, 45% yield) as a white solid.

LCMS (ESI) m/z: [M+H]⁺=224.1 ¹H NMR (400 MHz, DMSO-d₆) δ=13.09-13.06 (m, 1H), 8.18 (t, J=6.4 Hz, 1H), 7.64 (d, J=1.6 Hz, 1H), 7.60-7.57 (m, 1H), 7.29 (d, J=8.0 Hz, 1H), 4.45 (d, J=6.4 Hz, 2H), 3.91 (s, 2H) ppm

### Step 6: Preparation of 1,1,3-trioxo-4,5-dihydro-1λ6,4-benzothiazepine-8-carboxylic acid

To a solution of 3-oxo-4,5-dihydro-1,4-benzothiazepine-8-carboxylic acid (50 mg, 223.97 μmol) in MeOH (0.5 mL) and H₂O (0.5 mL) was added Oxone (275.37 mg, 447.93 μmol), the mixture was stirred at 30° C. for 2 h. The reaction mixture was poured into MeOH (5 mL), the solution was filtered and the filtrate was concentrated to give 1,1,3-trioxo-4,5-dihydro-1λ6,4-benzothiazepine-8-carboxylic acid (57 mg, 223.31 μmol, 99.71% yield) as a white solid.

Intermediate 11. 4-(2-methoxyethyl)-3-methylsulfonyl-benzoic acid

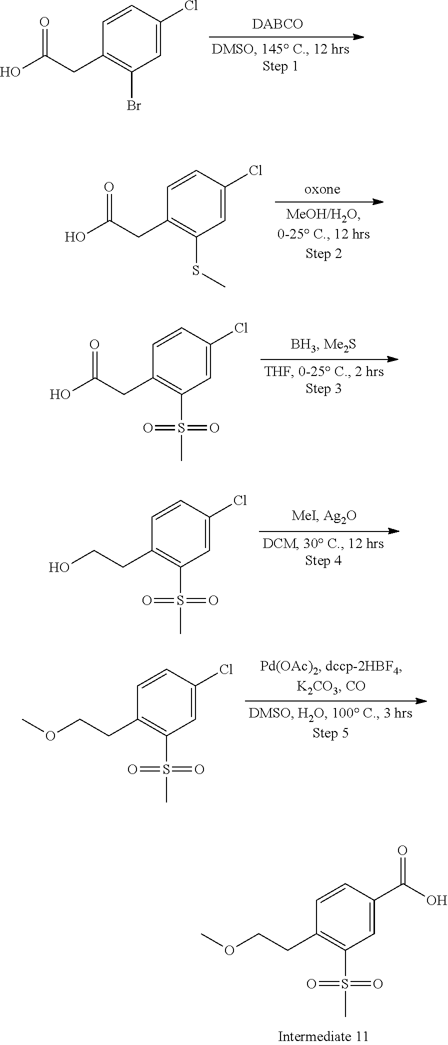

### Step 1: Preparation of 2-(4-chloro-2-methylsulfanyl-phenyl)acetic acid

A mixture of 2-(2-bromo-4-chlorophenyl)acetic acid (1 g, 4.01 mmol), CuI (763.36 mg, 4.01 mmol) and DABCO (899.20 mg, 8.02 mmol, 881.57 uL) in DMSO (10 mL) was stirred at 145° C. for 12 h under N₂. The reaction mixture was diluted with 1N HCl (300 mL) and filtered. The filtrate was extracted with DCM (300 mL×2). The organic layers were dried over anhydrous Na₂SO₄, filtered and concentrated to afford the residue. The residue was purified by column chromatography (Petroleum ether/Ethyl acetate=1/0 to 0/1). The eluent was concentrated to afford 2-(4-chloro-2-methylsulfanyl-phenyl)acetic acid (1.5 g, crude) as a yellow solid which was used directly to the next step.

### Step 2: Preparation of 2-(4-chloro-2-methylsulfonyl-phenyl)acetic acid

To a solution of 2-(4-chloro-2-methylsulfanyl-phenyl)acetic acid (500 mg, 2.31 mmol) in MeOH (3 mL) and H₂O (3 mL) was added ozone (4.26 g, 6.92 mmol) in H₂O (3 mL) at 0° C. The mixture was stirred at 25° C. for 12 h. The reaction mixture was diluted with sat·Na₂SO₃ (100 mL) and stirred for 10 min, then extracted with DCM (100 mL×3). The organic layers were dried over anhydrous Na₂SO₄, filtered and concentrated to afford the residue. The residue was purified by reversed phase (0.1% FA). The eluent was concentrated to afford 2-(4-chloro-2-methylsulfonyl-phenyl)acetic acid (200 mg, 0.804 mol, 35% yield) as a white solid. LCMS (ESI) m/z: [M+H]⁺=248.9.

¹H NMR (400 MHz, DMSO-d6) δ=12.62-12.54 (m, 1H), 7.91 (d, J=2.0 Hz, 1H), 7.78-7.78 (m, 1H), 7.55 (d, J=8.0 Hz, 1H), 4.05 (s, 2H), 3.26 (s, 3H) ppm.

### Step 3: Preparation of 2-(4-chloro-2-methylsulfonyl-phenyl)ethanol

To a solution of 2-(4-chloro-2-methylsulfonyl-phenylacetic acid (200 mg, 804.24 μmol) in THE (4 mL) was added a mixture of BH₃-Me2s (10 M, 402.12 uL) at 0° C. The mixture was stirred at 25° C. for 2 h. The reaction mixture was diluted with 1 N HCl (10 mL) and extracted with DCM (10 mL). The organic layers were dried over anhydrous Na₂SO₄, filtered and concentrated to afford the residue. The residue was purified by reversed phase (0.1% FA). The eluent was concentrated to afford 2-(4-chloro-2-methylsulfonyl-phenyl)ethanol (180 mg, 766.94 μmol, 96% yield) as colorless oil.

LCMS (ESI) m/z: [M+H]⁺=235.0.

¹H NMR (400 MHz, CDCl₃) δ=8.06 (d, J=2.4 Hz, 1H), 7.58-7.55 (m, 1H), 7.42 (d, J=8.0 Hz, 1H), 3.97-3.94 (m, 2H), 3.28-3.25 (m, 2H), 3.15 (s, 3H) ppm.

### Step 4: Preparation of 4-chloro-1-(2-methoxyethyl)-2-methylsulfonyl-benzene

To a solution of 2-(4-chloro-2-methylsulfonyl-phenyl)ethanol (80 mg, 0.341 mmol) in DCM (1 mL) was added Ag₂O (236.97 mg, 1.02 mmol) and MeI (241.91 mg, 1.70 mmol, 106 uL). The mixture was stirred at 30° C. for 12 h. The reaction mixture was diluted with H₂O (10 mL) and extracted with DCM (10 mL×2). The combined organic layers were dried over anhydrous Na₂SO₄, filtered and concentrated to afford the residue, which was purified by reversed phase (0.1% FA). The eluent was concentrated to afford 4-chloro-1-(2-methoxyethyl)-2-methylsulfonyl-benzene (60 mg, 0.241 mmol, 71% yield) as a yellow solid.

LCMS (ESI) m/z: [M+H]⁺=248.9.

¹H NMR (400 MHz, CDCl₃) δ=8.06 (d, J=2.4 Hz, 1H), 7.55-7.52 (m, 1H), 7.42 (d, J=8.4 Hz, 1H), 3.70-3.67 (m, 2H), 3.33-3.29 (m, 5H), 3.15 (s, 3H) ppm.

### Step 5: Preparation of 4-(2-methoxyethyl)-3-methylsulfonyl-benzoic acid ((intermediate 11)

A mixture of 4-chloro-1-(2-methoxyethyl)-2-methylsulfonyl-benzene (60 mg, 0.241 mmol), K₂CO₃ (50.0 mg, 0.362 mmol), dicyclohexyl(3-dicyciohexylphosphaniumylpropyl)phosphonium; ditetrafluoroborate (14.77 mg, 24.12 μmol) and Pd(OAc)₂ (2.71 mg, 12.06 μmol) in DMSO (1 mL) and H₂O (0.2 mL) was degassed and purged with CO for 3 times. The mixture was stirred at 100° C. for 3 h under CO (15 psi) atmosphere. The reaction mixture was diluted with MeOH (10 mL) and filtered. The filtrate was concentrated to get the residue. The residue was purified by reversed phase (0.1% FA). The eluent was concentrated to remove the ACN and lyophilized to afford 4-(2-methoxyethyl)-3-methylsulfonyl-benzoic acid (50 mg, 0.194 mmol, 80% yield) as a white solid.

LCMS (ESI) m/z: [M+H]⁺=259.0.

¹H NMR (400 MHz, CDCl₃) δ=8.78 (d, J=1.6 Hz, 1H), 8.28-8.26 (m, 1H), 7.61 (d, J=8.4 Hz, 1H), 3.77-3.74 (m, 2H), 3.45-3.42 (m, 2H), 3.33 (s, 3H), 3.19 (s, 3H) ppm.

### Intermediate 12. 4-(2-methoxyethyl)-3-methylsulfonyl-benzoic acid

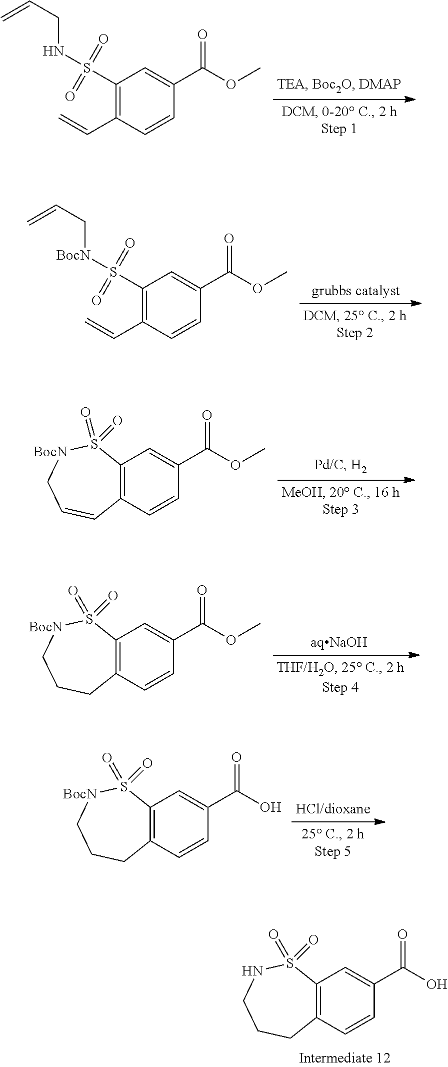

### Step 1: Preparation of methyl 3-[allyl(tert-butoxycarbonyl)sulfamoyl]-4-vinyl-benzoate

To a solution of methyl 3-(allylsulfarnoyl)-4-vinyl-benzoate (1.2 g, 4.27 mmol) (Prepared according to the method in FG-A4366) and DMAP (52.11 mg, 426.55 μmol) in DCM (20 mL) was added TEA (863.24 mg, 8.53 mmol, 1.19 mL) and Boc₂O (1.86 g, 8.53 mmol, 1.96 mL) at 0° C. The mixture was stirred at 20° C. for 2 h. It was poured into water (60 mL) and extracted with DCM (40 mL×3). The combined organic layers were washed with brine (40 mL), dried over Na₂SO₄, filtered and concentrated. The residue was purified by flash silica gel chromatography (ISCO®; 40 g SepaFlash® Silica Flash Column, Eluent of 0˜50% Ethylacetate/Petroleum ethergradient @50 mL/min). The fraction was concentrated in vacuum to give methyl 3-[allyl(tert-butoxycarbonyl)sulfamoyl]-4-vinyl-benzoate (1.5 g, 3.93 mmol, 92% yield) as a yellow oil.

¹H NMR (400 MHz, DMSO-d₆) δ=8.50 (d, J=2.0 Hz, 1H), 8.34-8.15 (m, 1H), 7.92 (d, J=8.4 Hz, 1H), 7.23-7.00 (m, 1H), 6.01-5.86 (m, 2H), 5.75-5.61 (m, 1H), 5.39-5.14 (m, 2H), 4.38 (d, J=4.8 Hz, 2H), 3.91 (s, 3H), 1.13 (s, 9H) ppm.

### Step 2: Preparation of 2-(tert-butyl) 8-methyl benzo[f][1,2]thiazepine-2,8(3H)-dicarboxylate 1,1-dioxide

A mixture of methyl 3-[allyl(tert-butoxycarbonyl)sulfamoyl]-4-vinyl-benzoate (1.5 g, 3.93 mmol) and benzylidene-[1,3-bis(2,4,6-trimethylphenyl)imidazolidin-2-ylidene]-dichloro-ruthenium;tricyclohexylphosphane (333.85 mg, 393.24 μmol) in DCM (80 mL) was degassed and purged with N₂ for 3 times. The mixture was stirred at 25° C. for 2 h under N₂ atmosphere. It was concentrated to remove DCM. The residue was purified by flash silica gel chromatography (ISCO®; 40 g SepaFlash® Silica Flash Column, Eluent of 0˜50% Ethylacetate/Petroleum ethergradient @50 mL/min). The fraction was concentrated in vacuum to give 2-(tert-butyl) 8-methyl benzo[f][1,2]thiazepine-2,8(3H)-dicarboxylate 1,1-dioxide (1.1 g, 2.77 mmol, 70% yield) as a yellow solid.

LCMS (ESI) m/z: [Br⁷⁹M+H]⁺=298.0

¹H NMR (400 MHz, DMSO-d₆) δ=8.43 (d, J=1.6 Hz, 1H), 8.32-8.17 (m, 1H), 7.82 (d, J=8.0 Hz, 1H), 6.75 (d, J=12.8 Hz, 1H), 6.39-6.18 (m, 1H), 4.95-4.57 (m, 2H), 3.92 (s, 3H), 1.11 (s, 9H) ppm.

### Step 3: Preparation of 2-(tert-butyl) 8-methyl 4,5-dihydrobenzo[f][1,2]thiazepine-2,8(3H)-dicarboxylate 1,1-dioxide

A mixture of 2-(tert-butyl) 8-methyl benzo[f][1,2]thiazepine-2,8(3H)-dicarboxylate 1,1-dioxide (500 mg, 1.41 mmol), Pd/C (50 mg, 10% purity) in MeOH (10 mL) was degassed and purged with H₂ for 3 times. The mixture was stirred at 20° C. for 18 h under H₂ atmosphere. It was filtered and concentrated to give 2-(tert-butyl) 8-methyl 4,5-dihydrobenzo[f][1,2]thiazepine-2,8(3H)-dicarboxylate 1,1-dioxide (4.1 g, 12.27 mmol, 96% yield) as a yellow oil.

LCMS (ESI) m/z: [Br⁷⁹M+H]⁺=300.0

¹H NMR (400 MHz, DMSO-d₆) δ=8.37 (d, J=2.0 Hz, 1H), 8.23-8.11 (m, 1H), 7.66 (d, J=8.0 Hz, 1H), 4.17-4.06 (m, 2H), 3.90 (s, 3H), 3.32-3.14 (m, 2H), 1.90-1.53 (m, 2H), 1.22 (s, 9H) ppm.

### Step 4: Preparation of 2-tert-butoxycarbonyl-1,1-dioxo-4,5-dihydro-3H-1λ6,2-benzothiazepine-8-carboxylic acid

To a solution of 2-(tert-butyl) 8-methyl 4,5-dihydrobenzo[f][1,2]thiazepine-2,8(3H)-dicarboxylate 1,1-dioxide (250 mg, 0.703 mmol) in THE (2.5 mL) and H₂O (2.5 mL) was added LiOH·H₂O (118.06 mg, 2.81 mmol). The mixture was stirred at 25° C. for 2 h. It was adjusted to pH=5 by aq·HCl (1 M) and extracted with EA (40 mL×3). The combined organic layers were washed with brine (30 mL), dried over Na₂SO₄, filtered and concentrated to give 2-tert-butoxycarbonyl-1,1-dioxo-4,5-dihydro-3H-1λ6,2-benzothiazepine-8-carboxylic acid (190 mg, 0.473 mmol, 67% yield) as a white solid. LCMS (ESI) m/z: [M+H]⁺=285.9

### Step 5: Preparation of 1,1-dioxo-2,3,4,5-tetrahydro-1λ6,2-benzothiazepine-8-carboxylic acid (Intermediate 12)

A mixture of 2-tert-butoxycarbonyl-1,1-dioxo-4,5-dihydro-3H-1λ6,2-benzothiazepine-8-carboxylic acid (180 mg, 0.527 mmol) in HCl/dioxane (4 M, 3 mL) was stirred at 25° C. for 2 h. It was concentrated to remove dioxane to give 1,1-dioxo-2,3,4,5-tetrahydro-1λ6,2-benzothiazepine-8-carboxylic acid (130 mg, 0.468 mmol, 89% yield, HCl) as a yellow solid.

¹H NMR (400 MHz, DMSO-d₆) δ=8.31 (d, J=1.6 Hz, 1H), 8.05-8.00 (m, 1H), 7.57-7.52 (m, 2H), 3.66 (br s, 2H), 3.22 (br d, J=3.2 Hz, 2H), 1.91-1.77 (m, 1H), 1.70 (br s, 2H) ppm.

### Intermediate 13. 2-methyl-1,1-dioxo-4,5-dihydro-3H-1λ6,2-benzothiazepine-8-carboxylic acid

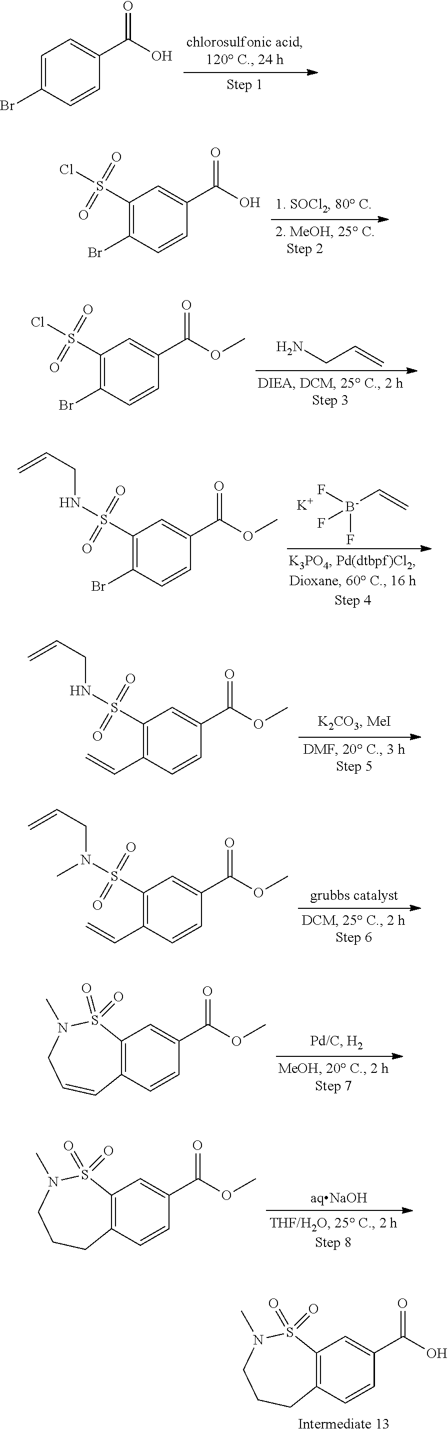

### Step 1: Preparation of 4-bromo-3-chlorosulfonyl-benzoic acid

A mixture of 4-bromobenzoic acid (10 g, 49.75 mmol) in HSO₃Cl (86.95 g, 0.746 mol, 49.7 mL) was stirred at 100° C. for 16 h. The reaction was stirred at 120° C. for another 16 h. It was poured into ice water (400 mL). A precipitate was formed and the mixture was filtered. The filtered cake was dried under vacuum to afford 4-bromo-3-chlorosulfonyl-benzoic acid (11 g, 36.72 mmol, 73% yield) as a gray solid.

LCMS (ESI) m/z: [Br⁷⁹M+H]⁺=300.0

¹H NMR (400 MHz, DMSO-d₆) δ=13.96 (br s, 1H), 8.46 (d, J=1.8 Hz, 1H), 7.79-7.80 (m, 2H) ppm.

### Step 2: Preparation of methyl 4-bromo-3-chlorosulfonyl-benzoate

To a mixture of 4-bromo-3-chlorosulfonyl-benzoic acid (11 g, 36.72 mmol) in SOCl₂ (43.69 g, 387.25 mmol, 28.64 mL) was stirred at 80° C. for 2 h. Then the mixture was concentrated to remove SOCl₂. MeOH (11 mL) was added. The mixture was stirred at 20° C. for 0.5 hr. It was poured into water (600 mL) and extracted with EA (300 mL×3). The combined organic layers were washed with brine (200 mL), dried over Na₂SO₄, filtered and concentrated to give methyl 4-bromo-3-chlorosulfonyl-benzoate (10 g, crude) as a yellow solid.

LCMS (ESI) m/z: [Br⁷⁸M+H]⁺=314.8

¹H NMR (400 MHz, DMSO-d₆) δ=9.31 (br s, 2H), 8.71-8.31 (m, 1H), 7.89-7.62 (m, 2H), 3.86 (s, 3H).

### Step 3: Preparation of methyl 3-(allylsulfamoyl)-4-bromo-benzoate

To a solution of methyl 4-bromo-3-chlorosulfonyl-benzoate (4 g, 12.76 mmol) and prop-2-en-1-amine (1.31 g, 14.03 mmol, 1.73 mL, HCl) in DCM (40 mL) was added DIEA (6.60 g, 51.03 mmol, 8.89 mL) at 0° C. Then then mixture was stirred at 25° C. for 2 h. It was poured into water (100 mL) and extracted with DCM (80 mL×3). The combined organic layers were washed with brine (80 mL), dried over Na₂SO₄, filtered and concentrated. The residue was purified by flash silica gel chromatography (ISCO®; 80 g SepaFlash® Silica Flash Column, Eluent of 0˜50% Ethylacetate/Petroleum ethergradient @ 100 mL/min). The fraction was concentrated in vacuum to give methyl 3-(allylsulfamoyl)-4-bromo-benzoate (4.1 g, 12.27 mmol, 96% yield) as a white solid.

¹H NMR (400 MHz, DMSO-d₆) δ=8.51-8.42 (m, 1H), 8.29 (br s, 1H), 8.01 (d, J=0.8 Hz, 2H), 5.77-5.52 (m, 1H), 5.17-5.06 (m, 1H), 5.03-4.93 (m, 1H), 3.89 (s, 3H), 3.57 (br d, J=4.8 Hz, 2H) ppm.

### Step 4: Preparation of methyl 3-(allylsulfamoyl)-4-vinyl-benzoate

A mixture of methyl 3-(allylsulfamoyl)-4-bromo-benzoate (3.1 g, 9.28 mmol), potassium; trifluoro(vinyl)boranuide (6.21 g, 46.38 mmol), ditert-butyl(cyclopentyl)phosphane; dichloropalladium; iron (604.6 mg, 0.928 mmol), and K₃PO₄ (5.91 g, 27.8 mmol) in dioxane (30 mL) and H₂O (6 mL) was degassed and purged with N₂ for 3 times. The mixture was stirred at 60° C. for 16 h under N₂ atmosphere. It was poured into water (100 mL) and extracted with EA (60 mL×3). The combined organic layers were washed with brine (60 mL), dried over Na₂SO₄, filtered and concentrated. The residue was purified by flash silica gel chromatography (ISCO®; 40 g SepaFlash® Silica Flash Column, Eluent of 0˜50% Ethylacetate/Petroleum ethergradient @ 80 mL/min). The fraction was concentrated in vacuum to give methyl 3-(allylsulfamoyl)-4-vinyl-benzoate (1.5 g, 5.33 mmol, 57% yield) as a white solid.

LCMS (ESI) m/z: [M+H]⁺=282.1

¹H NMR (400 MHz, CDCl₃) δ=8.62 (d, J=1.8 Hz, 1H), 8.29-8.11 (m, 1H), 7.69 (d, J=8.0 Hz, 1H), 7.62-7.47 (m, 1H), 5.92-5.77 (m, 1H), 5.73-5.53 (m, 2H), 5.22-4.97 (m, 2H), 4.84-4.57 (m, 1H), 3.69-3.41 (m, 2H) ppm.

### Step 5: Preparation of methyl 3-[allyl(methyl)sulfamoyl]-4-vinyl-benzoate

To a solution of methyl 3-(allylsulfamoyl)-4-vinyl-benzoate (200 mg, 0.711 mmol) and K₂CO₃ (196.5 mg, 1.42 mmol) in DMF (2 mL) was added MeI (201.81 mg, 1.42 mmol, 88.5 μL). The mixture was stirred at 20° C. for 3 h. It was poured into water (60 mL) and extracted with EA (30 mL×3). The combined organic layers were washed with brine (20 mL) and then dried over Na₂SO₄, filtered and concentrated. The residue was purified by flash silica gel chromatography (ISCO®; 12 g SepaFlash® Silica Flash Column, Eluent of 0˜50% Ethylacetate/Petroleum ethergradient (c) 50 mL/min). The fraction was concentrated in vacuum to give methyl 3-[allyl(methyl)sulfamoyl]-4-vinyl-benzoate (190 mg, 0.643 mmol, 90% yield) as a yellow oil.

LCMS (ESI) m/z: [M+H]⁺=296.0

¹H NMR (400 MHz, CDCl₃) δ=8.56 (d, J=1.6 Hz, 1H), 8.26-8.09 (m, 1H), 7.73 (d, J=8.4 Hz, 1H), 7.67-7.53 (m, 1H), 5.88-5.77 (m, 1H), 5.76-5.64 (m, 1H), 5.59-5.50 (m, 1H), 5.27-5.16 (m, 2H), 3.96 (s, 3H), 3.75 (d, J=8.4 Hz, 2H), 2.75 (s, 3H) ppm.

### Step 6: Preparation of methyl 2-methyl-1,1-dioxo-3H-1λ6,2-benzothiazepine-8-carboxylate

A mixture of methyl 3-[allyl(methyl)sulfamoyl]-4-vinyl-benzoate (190 mg, 0.643 mmol) and benzylidene-[1,3-bis(2,4,6-trimethylphenyl)imidazolidin-2-ylidene]-dichloro-ruthenium:tricyclohexylphosphane (54.61 mg, 64.33 μmol) in DCM 10 mL) was degassed and purged with N₂ for 3×. The mixture was stirred at 25° C. for 2 h under N₂ atmosphere. The residue was purified by flash silica gel chromatography (ISCO®; 12 g SepaFlash® Silica Flash Column, Eluent of ˜50% Ethylacetate/Petroleum ethergradient @ 30 mL/min). The fraction was concentrated in vacuum to give methyl 2-methyl-1,1-dioxo-3H-1λ6,2-benzothiazepine-8-carboxylate (130 mg, 0.486 mmol, 76% yield) as a white solid.

LCMS (ESI) m/z: [M+H]⁺=268.0

¹H NMR (400 MHz, DMSO-d₆) δ=8.38 (d, J=2.0 Hz, 1H), 8.26-8.10 (m, 1H), 7.78 (d, J=8.4 Hz, 1H), 6.72 (br d, J=13.2 z, 1H), 6.31-5.96 (m, 1H), 4.45-4.17 (m, 2H), 3.90 (s, 3H), 2.55 (s, 3H) ppm.

### Step 7: Preparation of methyl 2-methyl-1,1-dioxo-4,5-dihydro-3H-1λ, 6,2-benzothiazepine-8-carboxylate

A mixture of methyl 2-methyl-1,1-dioxo-3H-1λ6,2-benzothiazepine-8-carboxylate (130 mg, 0.488 mmol), Pd/C (13 mg, 10% purity) in MeOH (4 mL) was degassed and purged with H₂ for 3 times. Then the mixture was stirred at 20° C. for 2 h under H₂ atmosphere. It was filtered and concentrated to give methyl 2-methyl-1,1-dioxo-4,5-dihydro-3H-1λ6,2-benzothiazepine-8-carboxylate (110 mg, 0.408 mmol, 84% yield) as a white solid.

LCMS (ESI) m/z: [M+H]⁺=270.0

¹H NMR (400 MHz, CDCl₃) δ=8.56 (d, J=2.0 Hz, 1H), 8.17-8.03 (m, 1H), 7.38 (d, J=7.6 Hz, 1H), 3.95 (s, 3H), 3.92-3.59 (m, 2H), 3.45-3.23 (m, 2H), 2.85 (s, 3H), 1.91-1.80 (m, 3H) ppm.

### Step 8: Preparation of 2-methyl-1,1-dioxo-4,5-dihydro-3H-1λ,6,2-benzothiazepine-8-carboxylic acid (Intermediate 13)

To a solution of methyl 2-methyl-1,1-dioxo-4,5-dihydro-3H-1λ6,2-benzothiazepine-8-carboxylate (110 mg, 0.408 mmol) in THE (1 mL) and H₂O (1 mL) was added LiOH·H₂O (88.58 mg, 1.83 mmol). The mixture was stirred at 25° C. for 2 h. It was adjusted to PH=5 by aq·HCl (1 M) and extracted with EA (20 mL×3). The combined organic layers were washed with brine (20 mL) and then dried over Na₂SO₄, filtered and concentrated to give 2-methyl-1,1-dioxo-4,5-dihydro-3H-1λ6,2-benzothiazepine-8-carboxylic acid (80 mg, 0.313 mmol, 77% yield) as a white solid.

LCMS (ESI) m/z: [M+H]⁺=519.2

¹H NMR (400 MHz, DMSO-d₆) δ=8.27 (d, J=1.6 Hz, 1H), 8.14-8.01 (m, 1H), 7.59 (d, J=8.0 Hz, 1H), 3.75-3.55 (m, 2H), 3.23 (br s, 3H), 2.55 (s, 3H), 1.83-1.71 (m, 2H) ppm.

### Intermediate 14. 4-(difluoromethyl)-3-(methylsulfonyl)benzoic acid

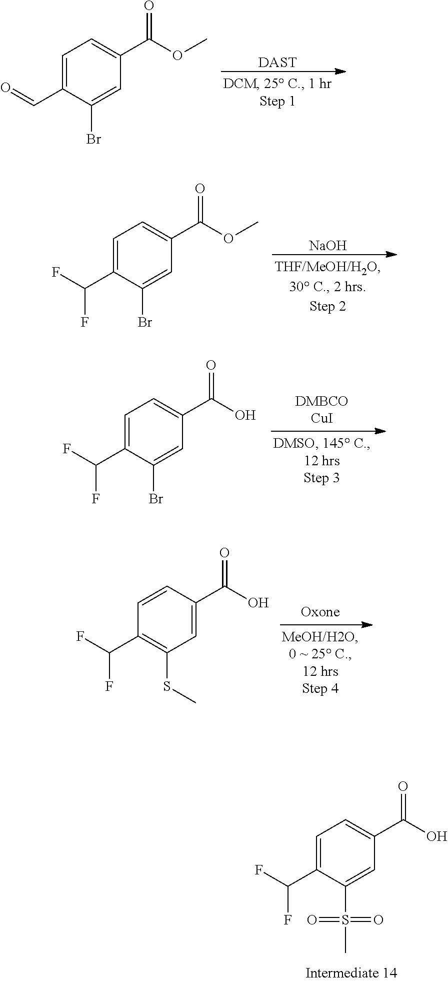

### Step 1: Preparation of methyl 3-bromo-4-(difluoromethyl)benzoate

To a solution of methyl 3-bromo-4-formylbenzoate (300 mg, 1.23 mmol) in DCM (3 mL) was added DAST (596.87 mg, 3.70 mmol, 489.24 uL). The mixture was stirred at 25° C. for 1 hr. The reaction mixture was diluted with sat.NaHCO₃ (20 mL) and extracted with DCM (20 mL). The organic layer was dried over anhydrous Na₂SO₄, filtered and concentrated to afford the residue. The residue was purified by column chromatography (Petroleum ether/Ethyl acetate=1/0 to 3/1). The eluent was concentrated to afford methyl 3-bromo-4-(difluoromethyl)benzoate (190 mg, 716.84 μmol, 58% yield) as yellow oil.

¹H NMR (400 MHz, CDCl₃) δ=8.28 (s, 1H), 8.09 (d, J=8.0 Hz, 1H), 7.75 (d, J=8.0 Hz, 1H), 7.06-6.79 (m, 1H), 3.96 (s, 3H) ppm.

### Step 2: Preparation of 3-bromo-4-(difluoromethyl)benzoic acid

To a solution of methyl 3-bromo-4-(difluoromethyl)benzoate (90 mg, 339.56 μmol) in THE/MeOH/H₂O=2/1/1 (1 mL) was added NaOH (27.16 mg, 679.11 μmol). The mixture was stirred at 30° C. for 2 h. The reaction mixture was diluted with 1N HCl (10 mL) and extracted with DCM (10 mL×2). The combined organic layers were dried over anhydrous Na₂SO₄, filtered and concentrated to afford 3-bromo-4-(difluoromethyl)benzoic acid (70 mg, 278.88 μmol, 82% yield) as yellow oil which was used directly to the next step.

### Step 3: Preparation of 4-(difluoromethyl)-3-methylsulfanyl-benzoic acid

A mixture of 3-bromo-4-(difluoromethyl)benzoic acid (50 mg, 0.199 mmol), DABCO (44.68 mg, 0.398 mmol, 44 uL) and CuI (37.93 mg, 0.199 mmol) in DMSO (0.5 mL) was stirred at 145° C. for 12 h. To the mixture was added 1N HCl to adjust the pH=5. The mixture was filtered. The filtrate was concentrated to get the residue. The residue was purified by reversed phase (0.1% FA). The eluent was concentrated to afford 4-(difluoromethyl)-3-methylsulfanyl-benzoic acid (30 mg, 0.137 mmol, 69% yield) as a yellow solid.

LCMS (ESI) m/z: [M+H]⁺=218.9

¹H NMR (400 MHz, CDCl₃) δ=8.10 (s, 1H), 8.02 (d, J=8.4 Hz, 1H), 7.75 (d, J=8.0 Hz, 1H), 7.15-6.87 (m, 1H), 2.58 (s, 3H) ppm.

### Step 4: Preparation of 4-(difluoromethyl)-3-methylsulfonyl-benzoic acid (Intermediate 14)

To a solution of 4-(difluoromethyl)-3-methylsulfanyl-benzoic acid (30 mg, 137.48 μmol) in MeOH (0.5 mL) was added a mixture of Oxone (169.03 mg, 274.95 μmol) in H₂O (0.5 mL) at 0° C. The mixture was stirred at 25° C. for 12 h. The reaction mixture was diluted with H₂O (10 mL) and extracted with DCM (10 mL). The organic layer was dried over anhydrous Na₂SO₄, filtered and concentrated to afford the residue. The residue was purified by reversed phase (0.1% FA). The eluent was concentrated to afford 4-(difluoromethyl)-3-(methylsulfonyl)benzoic acid (20 mg, 79.9 μmol, 58% yield) as a white solid.

LCMS (ESI) m/z: [M+H]⁺=250.9.

### Intermediate 15. 6-Methyl-5-(methylsulfonyl)nicotinic acid

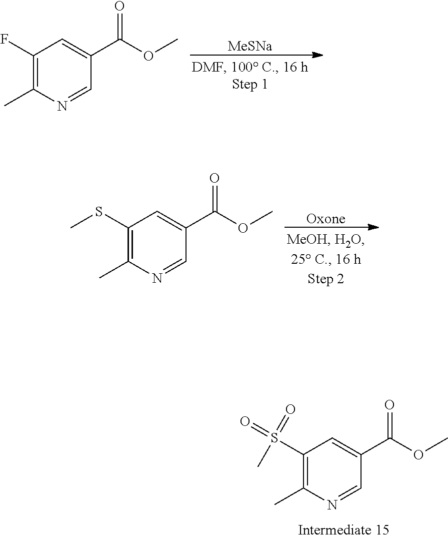

### Step 1: Preparation of 6-methyl-5-(methylthio)nicotinic acid

To a solution of methyl 5-fluoro-6-methyl-pyridine-3-carboxylate (300 mg, 1.77 mmol) in DMF (2 mL) was added sodium thiomethoxide (320.30 mg, 1.95 mmol). The mixture was stirred at 100° C. for 16 h. The reaction mixture was quenched with HCl (1 M) (40 mL) and extracted with EA/MeOH=15/1 (40 mL×5). The combined organic layer was dried over anhydrous Na₂SO₄, filtered and concentrated under reduced pressure to get residue. The residue was purified by reversed-phase HPLC (0.1% FA condition). The solution was concentrated under reduced pressure to remove MeCN and then lyophilized to afford 6 methyl-5-(methylthio)nicotinic acid (200 mg, 1.09 mmol, 62% yield) as a yellow solid.

LCMS (ESI) m/z: [M+H]⁺=183.9.

¹H NMR (400 MHz, DMSO-d₆) δ=14.14-12.40 (m, 1H), 8.69 (d, J=1.6 Hz, 1H), 7.94 (d, J=2.0 Hz, 1H), 2.55 (s, 3H), 2.50 (s, 3H) ppm.

### Step 2: Preparation of 6-methyl-6-(methylsulfonyl)nicotinic acid (Intermediate 15)

To a solution of B-methyl-5-(methylthio)nicotinic acid (30 mg, 163.73 μmol) in MeOH (1 mL) was added Oxone® (150.98 mg, 0.246 mmol) and H₂O (1 mL). The mixture was stirred at 25° C. for 16 h. The reaction mixture was dissolved with DMSO (5 mL) and then filtered to get the filtrate. The filtrate was purified by reversed-phase HPLC (0.1% FA condition). The solution was concentrated under reduced pressure to remove MeCN and then lyophilized to obtain 6-methyl-5-(methylsulfonyl)nicotinic acid (15 mg, 87.8 μmol, 41% yield) as white solid.

LCMS (ESI) m/z: [M+H]⁺=216.1.

¹H NMR (400 MHz, DMSO-d₆) δ=15.43-11.63 (m, 1H), 9.17 (d, J=2.0 Hz, 1H), 8.61 (d, J=2.0 Hz, 1H), 3.36 (s, 3H), 2.90 (s, 3H) ppm.

Intermediate 16. 3-chloro-4-methyl-6-methylsulfonyl-benzoic acid

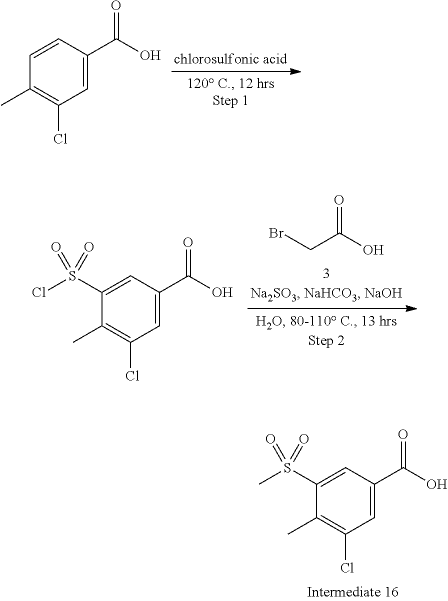

### Step 1: Preparation of 3-chloro-5-chlorosulfonyl-4-methylbenzoic acid

A mixture of 3-chloro-4-methylbenzoic acid (1 g, 5.86 mmol) in chlorosulfonic acid (10.25 g, 87.93 mmol, 5.85 mL) was stirred at 120° C. for 12 h. The reaction mixture was added to H₂O (20 mL) at 0° C. White solid was precipitated out from the mixture. The solid was collected by filtration and dried under reduced pressure to afford 3-chloro-5-chlorosulfonyl-4-methylbenzoic acid (1.2 g, 4.46 mmol, 76% yield) as a white solid.

¹H NMR (400 MHz, DMSO-d6) δ=8.30 (d, J=2.0 Hz, 1H), 7.85 (d, J=2.0 Hz, 1H), 2.63 (s, 3H) ppm.

### Step 2: Preparation of 3-chloro-4-methyl-6-methylsulfonyl-benzoic acid (intermediate 16)

To a solution of Na₂SO₃ (140.51 mg, 1.11 mmol) and NaHCO₃ (280.97 mg, 3.34 mmol, 130.08 uL) in H₂O (1.2 mL) was added 3-chloro-5-chlorosulfonyl-4-methylbenzoic acid (300 mg, 1.11 mmol) at 80° C. The mixture was stirred at 80° C. for 1 hr. Then 2-bromoacetic acid (309.8 mg, 2.23 mmol, 161 NL) and NaOH (89.19 mg, 2.23 mmol) were added and the mixture was stirred at 110° C. for 12 h. The reaction mixture was diluted with H₂O (10 mL), then added 1 N HCl to adjust the pH=3. The white solid was precipitated out from the mixture. The solid was collected by filtration and dried under reduced pressure to afford 3-chloro-4-methyl-5-methylsulfonyl-benzoic acid (120 mg, 0.483 mmol, 43% yield) as a white solid.

LCMS (ESI) m/z: [M+H]⁺=248.9

¹H NMR (400 MHz, DMSO-d8) δ=8.41 (d, J=1.6 Hz, 1H), 8.21 (d, J=1.6 Hz, 1H), 3.32 (s, 3H), 2.74 (s, 3H) ppm.

### Intermediate 17. 4-Chloro-3-fluoro-5-methylsulfonyl-benzoic acid

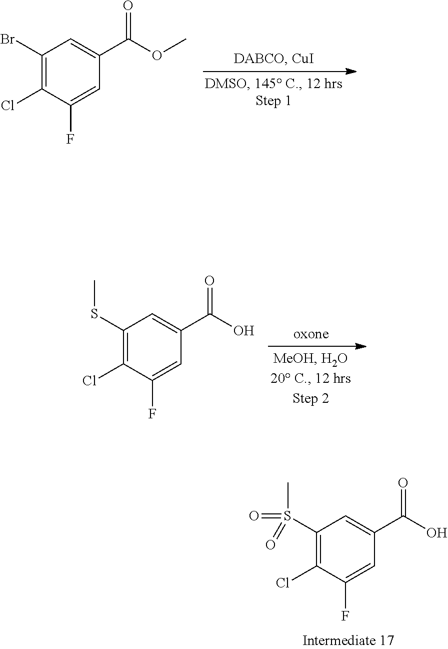

### Step 1: Preparation of 4-chloro-3-fluoro-5-methylsulfanyl-benzoic acid

A mixture of methyl 3-bromo-4-chloro-5-fluoro-benzoate (200 mg, 747.72 μmol), CuI (142.40 mg, 747.72 μmol) and DABCO (167.8 mg, 1.50 mmol, 164 μL) In DMSO (2 mL) was stirred at 145° C. for 12 h under N₂. The reaction mixture was filtered. The filtrate was purified by reversed-phase HPLC (0.1% FA condition). The desired fraction was lyophilized to give 4-chloro-3-fluoro-5-methylsulfonyl-benzoic acid (90 mg, 0.371 mmol, 50% yield) as a white solid.

LCMS (ESI) m/z: [M+H]⁺=220.9.

¹H NMR (400 MHz, DMSO-d₆) δ=7.66-7.56 (m, 2H), 2.59 (s, 3H) ppm.

### Step 2: Preparation of 4-chloro-3-fluoro-5-methylsulfonyl-benzoic acid (Intermediate 17)

To a solution of 4-chloro-3-fluoro-5-methylsulfanyl-benzoic acid (90 mg, 0.408 mmol) In H₂O (1 mL) and MeOH (2 mL) was added Oxone® (501.5 mg, 0.816 mmol). The reaction was stirred at 20° C. for 12 h under N₂. To the mixture was added saturated aqueous Na₂SO₃ (5 mL). The mixture was extracted with EA (5 mL×3). The combined organic layer was washed with brine (10 mL), dried over Na₂SO₄, filtered and concentrated to give 4-chloro-3-fluoro-5-methylsulfonyl-benzoic acid (40 mg, 0.158 mmol, 39% yield) as a white solid.

LCMS (ESI) m/z: [M+H]⁺=252.9.

¹H NMR (400 MHz, DMSO-d₆) δ=8.35 (s, 1H), 8.21-8.19 (m, 1H), 3.45 (s, 3H) ppm.

Intermediate 18. tert-Butyl ((2-chloro-1,6-naphthyridin-7-yl)methyl)carbamate

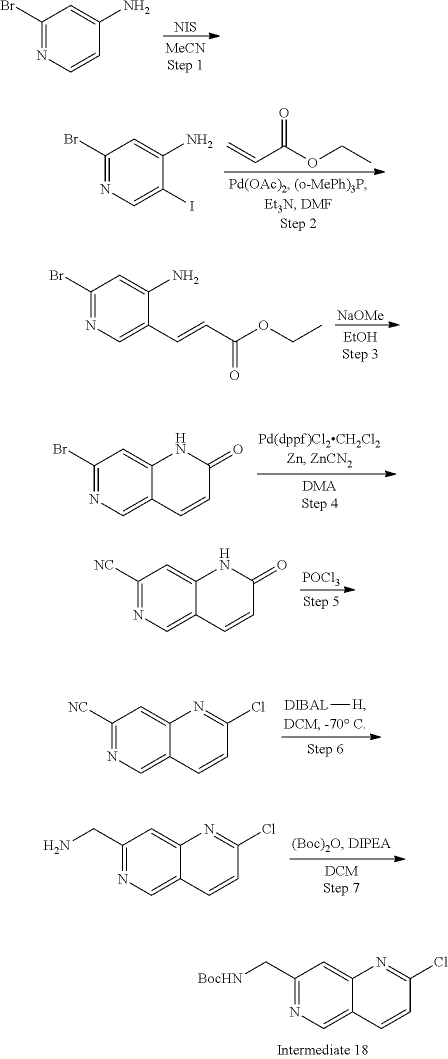

### Step 1. Preparation of 2-bromo-6-iodo-pyridin-4-amine

NIS (93.6 g, 416 mmol) was added to a solution of 2-bromopyridin-4-amine (60 g, 347 mmol) in MeCN (1.5 L) at 80° C. The reaction mixture was stirred at 80° C. for 36 h. The reaction mixture was concentrated under reduced pressure to give a residue. The residue was diluted with saturated Na₂SO₃ (1.5 L) and extracted with EA (1.5 L×2). The combined organic layers were washed with brine (1 L), dried over Na₂SO₄, filtered, and concentrated under reduced pressure to give a residue. The residue was purified by column chromatography (SiO₂, PE/EA=20:3) and concentrated under reduced pressure affording 2-bromo-5-iodo-pyridin-4-amine (65 g, 217 mmol) as a light-yellow solid.

¹H NMR (400 MHz, CDCl₃) δ=8.31 (s, 1H), 6.79 (s, 1H), 4.75 (br s, 2H) ppm.

### Step 2. Preparation of ethyl-3-(4-amino-8-bromo-3-pyridyl)prop-2-enoate

Ethyl prop-2-enoate (45.1 mL, 415 mmol), Et₃N (43.3 mL, 311 mmol), Pd(OAc)₂ (2.3 g, 10.4 mmol), and tris-o-tolylphosphane (6.3 g, 20.7 mmol) was added to a solution of 2-bromo-5-iodo-pyridin-4-amine (62 g, 207 mmol) in DMF (620 mL). The mixture was stirred at 100° C. for 3 h. The reaction mixture was diluted with water (4 L) and extracted with EA (2 L×2). The combined organic layers were washed with brine (2 L), dried over Na₂SO₄, filtered, and concentrated under reduced pressure to give a residue. The residue was purified by column chromatography (SiO₂, PE/EA=20:3) and concentrated under reduced pressure affording ethyl-3-(4-amino-6-bromo-3-pyridyl)prop-2-enoate (50 g, 170 mmol) as a light yellow solid.

LCMS (ESI) m/z: [79BrM+H]+=271.1.

¹H NMR (400 MHz, DMSO-d6) δ=8.23 (s, 1H), 7.73 (d, J=16.0 Hz, 1H), 6.90-6.67 (m, 3H), 6.52 (d, J=16.0 Hz, 1H), 4.18 (d, J=7.2 Hz, 2H), 1.25 (d, J=7.2 Hz, 3H) ppm.

### Step 3. Preparation of 7-bromo-1,6-naphthyridin-2(1H)-one

Sodium thiomethoxide (24.2 mL, 380 mmol) was added to a solution of ethyl-3-(4-amino-6-bromo-3-pyridyl)prop-2-enoate (40 g, 148 mmol) in EtOH (200 mL). The reaction mixture was stirred at 80° C. for 2 h. The reaction mixture was diluted with water (400 mL) and then neutralized with 1N HCl to pH=7.0. The solid was filtered and the filter cake was washed with water (50 mL). The filter cake was concentrated under reduced pressure affording 7-bromo-1,6-naphthyridin-2(1H)-one (22 g, 96.8 mmol) as an off-white solid.

LCMS (ESI) m/z: [79BrM+H]+=224.9.

¹H NMR (400 MHz, DMSO-d6) δ=12.08 (br s, 1H), 8.65 (s, 1H), 7.99 (d, J=9.6 Hz, 1H), 7.36 (s, 1H), 6.62 (d, J=9.6 Hz, 1H) ppm.

### Step 4. Preparation of 2-oxo-1,2-dihydro-1,6-naphthyridine-7-carbonitrile

Zinc powder (406.80 mg, 6.22 mmol) was added to a solution of 7-bromo-1,6-naphthyridin-2(1H)-one (7 g, 31.1 mmol), Zn(CN)₂ (3.95 mL, 62.2 mmol), and Pd(dppt)Cl₂·CH₂Cl₂ (5.08 g, 6.22 mmol) in DMA (140 mL). The reaction mixture was degassed and purged with N₂ three times then the mixture was stirred at 120° C. for 2 h. The reaction mixture was diluted with water (200 mL) and extracted with DCM/isopropanol (v/v=3:1) (150 mL×2). The combined organic layers were filtered, washed with brine (200 mL), dried over Na₂SO₄, filtered, and concentrated under reduced pressure to give a residue. The residue was purified by column chromatography (SiO₂, PE/EA=1:1) and concentrated under reduced pressure affording 2-oxo-1,2-dihydro-1,6-naphthyridine-7-carbonitrile (3 g, 17.5 mmol) as an off-white solid.

¹H NMR (400 MHz, DMSO-d6) δ=12.37 (br s, 1H), 8.97 (s, 1H), 8.09 (d, J=9.6 Hz, 1H), 7.67 (s, 1H), 6.77 (d, J=9.6 Hz, 1H) ppm.

### Step 6. Preparation 2-chloro-1,6-naphthyridine-7-carbonitrile

A mixture of 2-oxo-1,2-dihydro-1,6-naphthyridine-7-carbonitrile (3.0 g, 17.5 mmol) and POCl₃ (30 mL, 323 mmol) was stirred at 80° C. for 2 h. The reaction mixture was poured into H₂O (2 L) and adjusted to pH=7 with NaHCO₃. The solution was extracted with EA (1.5 L×2), the combined organic layers were washed with brine (2 L), dried over Na₂SO₄, filtered, and concentrated under reduced pressure to afford 2-chloro-1,6-naphthyridine-7-carbonitrile (1.1 g, 5.76 mmol) as a brown solid.

LCMS (ESI) m/z: [M+H]⁺=190.1.

¹H NMR (400 MHz, DMSO-d6) δ=9.58 (d, J=0.8 Hz, 1H), 8.79 (d, J=0.8 Hz, 1H), 8.69 (s, 1H), 8.00 (d, J=8.8 Hz, 1H) ppm.

### Step 6. Preparation of (2-chloro-1,6-naphthyridin-7-yl)methanamine

To a solution of 2-chloro-1,6-naphthyridine-7-carbonitrile (25 g, 131.86 mmol) in DCM (1000 mL) was added DIBAL-H (1 M, 329.64 mL, 2.5 eq) dropwise at −70° C. under N₂. The reaction mixture was stirred at −70′ C for 2 h. The reaction mixture was quenched with water (500 mL) and sat. potassium sodium tartrate (1500 mL) and stirred for an additional 30 min. The mixture was extracted with DCM:MeOH=10:1 (6000 mL×3). The combined organic layers were dried over Na₂SO₄, filtered and concentrated under reduced pressure to give (2-chloro-1,6-naphthyridin-7-yl)methanamine (51 g, crude) as a brown solid, which was used for the next step directly.

LCMS (ESI) m/z: [³⁵ClM+H]⁺=194.2

### Step 7: Preparation of tert-butyl ((2-chloro-1,6-naphthyridin-7-yl)methyl)carbamate (Intermediate 18)

To a solution of (2-chloro-1,6-naphthyridin-7-yl)methanamine (51 g, 263.4 mmol) in DCM (1500 mL) was added (Boc)₂O (172.45 g, 790.16 mmol) and DIEA (102.12 g, 790.16 mmol). The mixture was stirred at 25° C. for 16 h. The reaction mixture was diluted with water (1500 mL) and then filtered. The filtrate was extracted with DCM (1000 mL×3). The combined organic layers were washed with brine (1500 mL), dried over Na₂SO₄, filtered and concentrated under reduced pressure to give a residue. The residue was purified by column chromatography (SiO₂, Petroleum ether/Ethyl acetate=10/1 to 2/1 to 1/3) and the eluent was concentrated under reduced pressure to tert-butyl ((2-chloro-1,6-naphthyridin-7-yl)methyl)carbamate (21 g, 64.34 mmol, 24% yield) as a light yellow solid.

LCMS (ESI) m/z: [M+H]+=293.9.

¹H NMR (400 MHz, DMSO-d₆) δ=9.37 (s, 1H), 8.62 (d, J=8.4 Hz, 1H), 7.71 (d, J=8.4 Hz, 1H), 7.58-7.53 (m, 2H), 4.39 (d, J=6.4 Hz, 2H), 4.20-4.25 (m, 2H), 1.41 (s, 9H) ppm.

### Intermediate 19: [2-[6-(2,2-difluorocyclopropyl)-2-pyridyl]-1,6-naphthyridin-7-yl]methanamine

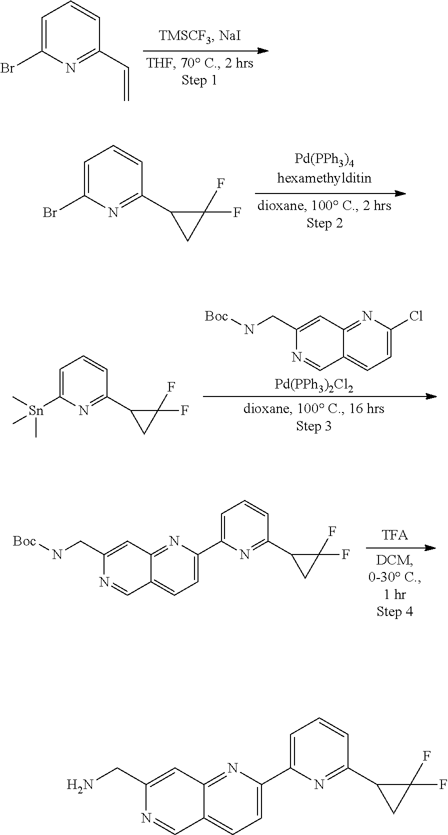

### Step 1: Preparation of 2-bromo-6-(2,2-difluorocyclopropyl)pyridine

To a mixture of 2-Bromo-6-ethenylpyridine (500 mg, 2.72 mmol) and NaI (81.45 mg, 0.543 mmol) in THF (4 mL) was added a solution of TMSCF₃ (1.55 g, 10.87 mmol) In THF (1 mL) over 1 h at 70° C. under N₂. The mixture was stirred at 70° C. for 1 h under N₂. The residue was purified by silica gel chromatography (PE-PE/EA=50/1). The eluent was concentrated under reduced pressure to afford 2-bromo-6-(2,2-difluorocyclopropyl)pyridine (570 mg, 2.44 mmol, 90% yield) as yellow oil.

LCMS (ESI) m/z: [M+H]⁺=233.9.

¹H NMR (400 MHz, CDCl₃) δ=7.52-7.48 (m, 1H), 7.39-7.37 (m, 1H), 7.19 (d, J=7.6 Hz, 1H), 2.95-2.84 (m, 1H), 2.21-2.12 (m, 1H), 1.89-1.83 (m, 1H) ppm.

### Step 2: Preparation of [6-(2,2-difluorocyclopropyl)-2-pyridyl]-trimethylstannane

A mixture of 2-bromo-6-(2,2-difluorocyclopropyl)pyridine (100 mg, 427.28 μmol). HEXAMETHYLDITIN (279.97 mg, 854.55 μmol, 177.20 uL) and Pd(PPh₃)₄ (49.37 mg, 42.73 μmol) in dioxane (2 mL) was stirred at 100° C. for 2 h under N₂. The mixture was filtered and concentrated under reduced pressure to afford [8-(2,2-difluorocyclopropyl)-2-pyridyl]-trimethyistannane (170 mg, crude) as brown oil.

LCMS (ESI) m/z: [M+H]⁺=320.1.

### Step 3: Preparation of tert-butyl N-[[2-[6-(2,2-difluorocyclopropyl)-2-pyridyl]-1,6-naphthyridin-7-yl]methyl]carbamate

A mixture of tert-butyl ((2-chloro-1,6-naphthyridin-7-yl)methyl)carbamate (50 mg, 170.21 μmol), [6-(2,2-difluorocyclopropyl)-2-pyridyl]-trimethylstannane (163 mg, 0.511 mmol) and Pd(PPh₃)₂Cl₂ (11.95 mg, 17.02 μmol) in dioxane (1 mL) was stirred at 100° C. for 18 h under N₂. The mixture was poured into sat. KF (10 mL) and stirred at 20° C. for 30 min. The mixture was extracted with EA (10 mL×3). The combined organic layers were dried over anhydrous Na₂SO₄, filtered and concentrated under reduced pressure to afford a residue. The residue was purified by silica gel chromatography (PE/EA=10/1-EA). The eluent was concentrated under reduced pressure to afford tert butyl N-[[2-[6-(2,2-difluorocyclopropyl)-2-pyridyl]-1,8-naphthyridin-7-yl]methyl]carbamate (30 mg, 72.74 μmol, 43% yield) as yellow solid.

LCMS (ESI) m/z: [M+H]⁺=413.3.

### Step 4: Preparation of [2-[6-(2,2-difluorocyclopropyl)-2-pyridyl]-1,6-naphthyridin-7-yl]methanamine (Intermediate 19)

To a mixture of tert-butyl N-[[2-[6-(2,2-difluorocyclopropyl)-2-pyridyl]-1,6-naphthyridin-7-yl]methyl]carbamate (30 mg, 72.74 μmol) in DCM (1 mL) was added TFA (462 mg, 4.05 mmol, 0.3 mL) at 0° C. The mixture was stirred at 30° C. for 1 hr. The mixture was concentrated under reduced pressure to afford [2-[6-(2,2-difluorocyclopropyl)-2-pyridyl]-1,6-naphthyridin-7-yl]methanamine (31 mg, 72.71 μmol, 100% yield, TFA salt) as yellow solid.

LCMS (ESI) m/z: [M+H]⁺=313.2.

### Intermediate 20. (2-(2-(2,2-difluorocyclopropyl)pyrimidin-4-yl)-1,6-naphthyridin-7-yl)methanaminemate

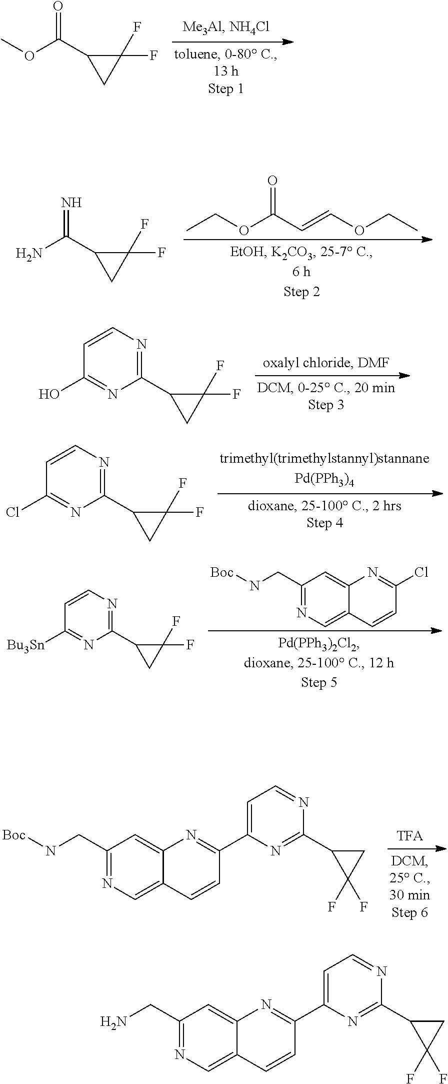

### Step 1: Preparation of 2,2-difluorocyclopropanecarboximidamide

To a mixture of NH₄Cl (6.88 g, 128.59 mmol) in toluene (50 mL) was added a solution of Al(CH₃)₃ (2 M, 64.29 mL) at 0° C. Then the mixture was stirred at 25° C. for 1 hr. To the solution was added methyl 2,2-difluorocyclopropanecarboxylate (3.5 g, 25.72 mmol) at 0° C., and then the solution was stirred at 80° C. for 12 h. A heavy white solid formed. The reaction mixture was cooled to 0° C. MeOH (50 mL) was added and then stirred for 10 min. The mixture was filtered. The filtrate was concentrated in vacuum to give 2,2-difluorocyclopropanecarboximidamide (3 g, crude) as a white solid which was used directly.

### Step 2: Preparation of 2-(2,2-difluorocyclopropyl)pyrimidin-4-ol

To a mixture of 2,2-difluorocyclopropanecarboximidamide (3.00 g, 24.97 mmol) in EtOH (40 mL) was added K₂CO₃ (6.90 g, 49.94 mmol) in one portion at 25° C. under N₂. The mixture was stirred at 25° C. for 10 min. then (E)-ethyl 3-ethoxyacrylate (1.2 g, 8.32 mmol, 1.20 mL) was added at 25° C. The mixture was stirred at 75° C. for 6 h. The reaction mixture was filtered and the filtrate was concentrated in vacuum. The mixture was purified by silica gel chromatography (DCM/MeOH=20/1). The eluent was concentrated to afford 2-(2,2-difluorocyclopropyl)pyrimidin-4-ol (500 mg, 2.90 mmol, 35% yield) as white solid.

LCMS (ESI) m/z: [M+H]⁺=173.2.

¹H NMR (400 MHz, CDCl₃) δ=8.04-7.95 (m, 1H), 6.43-8.35 (m, 1H), 2.84-2.69 (m, 1H), 2.51-2.39 (m, 1H), 2.00-1.88 (m, 1H) ppm.

### Step 3: Preparation of 4-chloro-2-(2,2-difluorocyclopropyl)pyrimidine

To a mixture of 2-(2,2-difluorocyclopropyl)pyrimidin-4-ol (350 mg, 2.03 mmol) and DMF (14.9 mg, 0.203 mmol, 15.8 uL) in DCM (6 mL) was added oxalyl chloride (518 mg, 4.07 mmol, 356 μL) in one portion at 0° C. under N₂. The mixture was stirred at 25° C. for 20 min. The mixture was added to sat. NaHCO₃ (50 mL) at 0° C. The aqueous phase was extracted with DCM (50 mL×2). The combined organic phase was washed with brine (50 mL×1), dried with anhydrous Na₂SO₄, filtered and concentrated in vacuum. The residue was purified by silica gel chromatography (PE/EA=10/1). The eluent was concentrated to afford 4-chloro-2-(2,2-difluorocyclopropyl)pyrimidine (150 mg, 0.787 mmol, 39% yield) as light yellow oil.

LCMS (ESI) m/z: [M+H]⁺=190.9, 192.9.

### Step 4: Preparation of 2-(2,2-difluorocyclopropyl)-4-(tributylstannyl)pyrimidine

To a mixture of 4-chloro-2-(2,2-difluorocyclopropyl)pyrimidine (100 mg, 0.525 mmol) and trimethyl(trimethylstannyl)stannane (343.8 mg, 1.05 mmol, 218 μL) In dioxane (2 mL) was added Pd(PPh₃)₄ (60.63 mg, 52.47 μmol) in one portion at 25° C. under N₂. The mixture was stirred at 100° C. for 2 h. The mixture was poured into water (10 mL) and extracted with ethyl acetate (10 mL×2). The combined organic phase was washed with brine (10 mL×1), dried with anhydrous Na₂SO₄, filtered and concentrated in vacuum to afford 2-(2,2-difluorocyclopropyl)-4-(tributylstannyl)pyrimidine (150 mg, crude) as yellow oil.

LCMS (ESI) m/z: [M+H]⁺=320.9.

### Step 6: Preparation tert-butyl ((2-(2-(2,2-difluorocyclopropyl)pyrimidin-4-yl)-1,6-naphthyridin-7-yl)methyl)carbamate

To a mixture of 2-(2,2-difluorocyclopropyl)-4-(tributylstannyl)pyrimidine (147 mg, 0.460 mmol) and tert-butyl ((2-chloro-1,6-naphthyridin-7-yl)methyl)carbamate (90 mg, 0.308 mmol) in dioxane (2 mL) was added Pd(PPh₃)₂Cl₂ (21.51 mg, 30.64 μmol) in one portion at 25° C. under N₂. The mixture was stirred at 100° C. for 12 h. The mixture was poured into water (30 mL) and extracted with ethyl acetate (20 mL×2). The combined organic phase was washed with brine (20 mL×1), dried with anhydrous Na₂SO₄, filtered and concentrated in vacuum. The residue was purified by flash silica gel chromatography (PE/EA=3/1). The eluent was concentrated to afford tert-butyl ((2-(2-(2,2-difluorocyclopropyl)pyrimidin-4-yl)-1,6-naphthyridin-7-yl)methyl)carbamate (90 mg, 0.218 mmol, 71% yield) as yellow solid.

LCMS (ESI) m/z: [M+H]⁺=414.0.

¹H NMR (400 MHz, DMSO-d6) δ=9.50-9.43 (m, 1H), 9.08-9.01 (m, 1H), 8.84-8.78 (m, 1H), 8.73-8.67 (m, 1H), 8.50-8.44 (m, 1H), 7.88-7.80 (m, 1H), 7.72-7.80 (m, 1H), 4.51-4.42 (m, 2H), 2.30-2.13 (m, 1H), 1.52-1.41 (m, 9H), 1.41-1.21 (m, 2H) ppm.

### Step 6: Preparation of (2-(2-(2,2-difluorocyclopropyl)pyrimidin-4-yl)-1,6-naphthyridin-7-yl)methanaminemate

To a mixture of tert-butyl ((2-(2-(2,2-difluorocyclopropyl)pyrimidin-4-yl)-1,6-naphthyridin-7-yl)methyl)carbamate (90 mg, 0.218 mmol) in DCM (1 mL) was added TFA (770.0 mg, 6.75 mmol, 500 μL) in one portion at 25° C. under N₂. The mixture was stirred at 25° C. for 30 min. The mixture was poured into ice-water (20 mL) and extracted with ethyl acetate (20 mL×1). The organic phase was discarded. To the aqueous phase was added sat·NaHCO₃ to adjust pH=8. Then the aqueous phase was extracted with ethyl acetate (20 mL×2). The combined organic phase was washed with brine (10 mL×1), dried with anhydrous Na₂SO₄, filtered and concentrated in vacuum to afford (2-(2-(2,2-difluorocyclopropyl)pyrimidin-4-yl)-1,6-naphthyridin-7-yl)methanaminemate (70 mg, crude) as light yellow solid, which was used directly without purification.

### Intermediate 21. [2-[6-(2,2-difluoro-1-methyl-cyclopropyl)-2-pyridyl]-1,6-naphthyridin-7-yl]methanamine

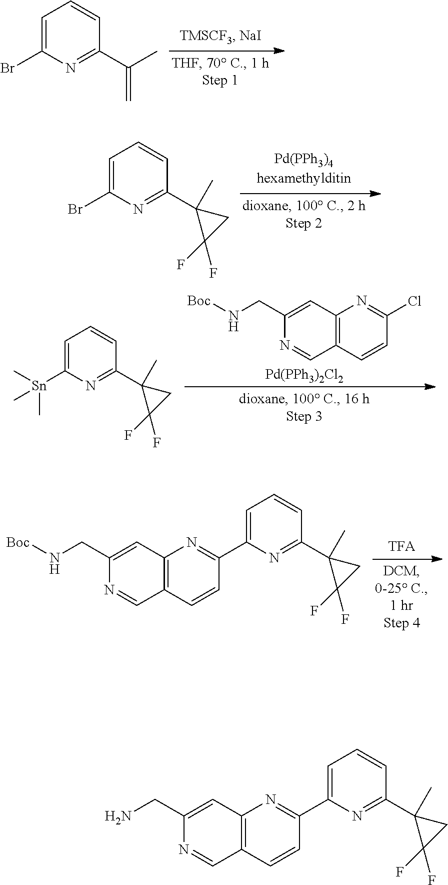

### Step 1: Preparation of 2-bromo-6-(2,2-difluoro-1-methyl-cyclopropyl)pyridine

To a mixture of 2-bromo-6-isopropenyl-pyridine (100 mg, 504.90 μmol) and NaI (15.14 mg, 100.98 μmol) in THE (0.8 mL) was added TMSCF₃ (287.19 mg, 2.02 mmol) dropwise over 30 min at 70° C. under N₂. The mixture was stirred at 70° C. for 30 min under N₂. The mixture was concentrated under reduced pressure to afford a residue. The residue was purified by silica gel chromatography (PE-PE/EA=20/1). The eluent was concentrated under reduced pressure to afford 2-bromo-6-(2,2-difluoro-1-methyl-cyclopropyl)pyridine (125 mg, 0.504 mmol, 100% yield) as yellow oil.

LCMS (ESI) m/z: [M+H]⁺=247.9.

¹H NMR (400 MHz, CDCl₃) δ=7.56-7.50 (m, 1H), 7.40-7.37 (m, 1H), 7.30 (d, J=7.6 Hz, 1H), 2.28-2.21 (m, 1H), 1.63-1.59 (m, 3H), 1.48-1.41 (m, 1H) ppm.

### Step 2: Preparation of [6-(2,2-difluoro-1-methylcyclopropyl)-2-pyridyl]-trimethyl-stannane

A mixture of 2-bromo-6-(2,2-difluoro-1-methyl-cyclopropyl)pyridine (100 mg, 403.12 μmol), HEXAMETHYLDITIN (264.15 mg, 0.806 mmol, 187 μL) and Pd(PPh₃)₄ (48.58 mg, 40.31 μmol) in dioxane (2 mL) was stirred at 100° C. for 2 h under N₂. The mixture was filtered and the filtrate was concentrated under reduced pressure to afford [6-(2,2-difluoro-1-methylcyctopropyl)-2-pyridyl]-trimethyl-stannane (210 mg, crude) as black brown oil.

LCMS (ESI) m/z: [M+H]⁺=334.0.

### Step 3: Preparation of tert-butyl N-[[2-[6-(2,2-difluoro-1-methyl-cyclopropyl)-2-pyridyl]-1,6-naphthyridin-7-yl]methyl]carbamate

A mixture of tert-butyl tert-butyl ((2-chloro-1,6-naphthyridin-7-yl)methyl)carbamate (80 mg, 0.204 mmol), [6-(2,2-difluoro-1-methylcyclopropyl)-2-pyridyl]-trimethyl-stannane (203.4 mg, 0.613 mmol) and Pd(PPh₃)₂Cl₂ (14.34 mg, 20.43 μmol) in dioxane (1 mL) was stirred at 100° C. for 16 h under N₂. The mixture was poured into sat. KF (10 mL) and stirred at 20° C. for 30 min. The mixture was extracted with EA (10 mL×3). The combined organic layers were dried over anhydrous Na₂SO₄, filtered and concentrated under reduced pressure to afford a residue. The residue was purified by silica gel chromatography (PE/EA=10/1-EA). The eluent was concentrated under reduced pressure to afford tert-butyl N-[[2-[6-(2,2-difluoro-1-methyl-cyclopropyl)-2-pyridyl]-1,6-naphthyridin-7-yl]methyl]carbamate (42 mg, 98.49 μmol, 48% yield) as yellow solid.

LCMS (ESI) m/z: [M+H]⁺=427.0.

### Step 4: Preparation of [2-[6-(2,2-difluoro-1-methyl-cyclopropyl)-2-pyridyl]-1,6-naphthyridin-7-yl]methanamine (Intermediate 21)

To a solution of tert-butyl N-[[2-[6-(2,2-difluoro-1-methyl-cyclopropyl)-2-pyridyl]-1,6-naphthyridin-7-yl]methyl]carbamate (42 mg, 98.49 μmol) in DCM (1 mL) was added TFA (462.0 mg, 4.05 mmol, 0.3 mL) at 0° C. The mixture was stirred at 25° C. for 1 hr. The mixture was concentrated under reduced pressure to afford [2-[6-(2,2-difluoro-1-methyl-cyclopropyl)-2-pyridyl]-1,6-naphthyridin-7-yl]methanamine (43 mg, 97.65 μmol, 99% yield, TFA salt) as yellow solid.

LCMS (ESI) m/z: [M+H]⁺=327.0.

### Intermediate 22. 1-Imino-1-oxo-3,5-dihydro-2H-4,1λ6-benzoxathiepine-8-carboxylic acid

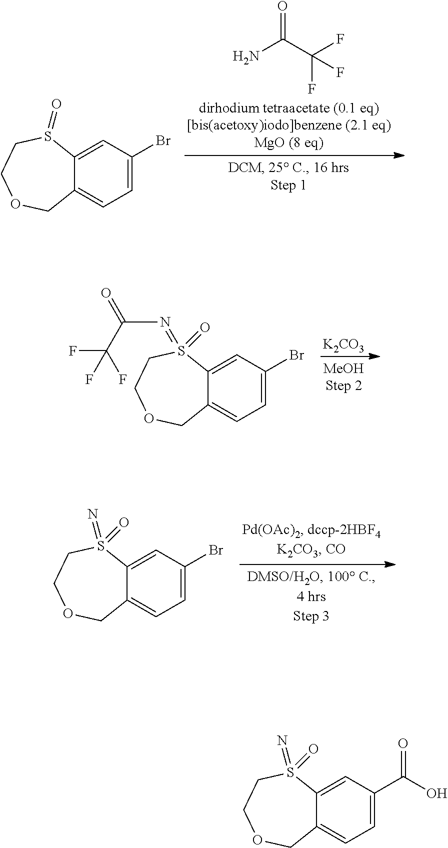

### Step 1: Preparation of N-(8-bromo-1-oxo-3,5-dihydro-2H-4,1λ6-benzoxathiepin-1-ylidene)-2,2,2-trifluoro-acetamide

A mixture of 8 8-bromo-3,5-dihydro-2H-4,1λ4-benzoxathiepine 1-oxide (50 mg, 191.47 μmol), 2,2,2-trifluoroacetamide (64.93 mg, 574.42 μmol,), [acetoxy(phenyl)-λ3-iodanyl] acetate (129.51 mg, 402.09 μmol) and MgO (46.30 mg, 1.15 mmol) in DCM (3 mL) was stirred at 25° C. for 5 min. Then diacetoxyrhodium (8.46 mg, 19.15 μmol) was added to the mixture and the mixture was stirred at 25° C. under N₂ for 18 h. The reaction mixture was diluted with MeOH (3 mL) to give N-(8-bromo-1-oxo-3,5-dihydro-2H-4,1λ6-benzoxathiepin-1-ylidene)-2,2,2-trifluoro-acetamide (71 mg, 190.78 μmol, 100% yield) as a yellow liquid which was used for the next step directly.

LCMS (ESI) m/z=[M+H]⁺=373.2.

### Step 2: Preparation of 8-bromo-1-imino-3,5-dihydro-2H-4,1λ6-benzoxathiepine 1-oxide

A mixture of N-(8-bromo-1-oxo-3,5-dihydro-2H-4,1λ6-benzoxathiepin-1-ylidene)-2,2,2-trifluoro-acetamide (70 mg, 188.09 μmol) in MeOH (3 mL) was added K₂CO₃ (181.97 mg, 1.32 mmol) and the mixture was stirred at 25° C. for 4 h. The mixture was diluted with water (10 mL) and filtered to remove the precipitate. The filtrate was separated and the aqueous layer was extracted with DCM (10 mL). The combined organic phase was washed with brine (10 mL), dried over anhydrous Na₂SO₄, filtered and the filtrate was concentrated under vacuum. The residue was purified by column chromatography (SiO₂, PE:EtOAc=20:1-1:1) to give 8-bromo-1-Imino-3,5-dihydro-2H-4,1λ6-benzoxathiepine 1-oxide (40 mg, 137.65 μmol, 73% yield) as a white solid.

LCMS (ESI) m/z=[M+H]⁺=277.2.

¹H NMR (400 MHz, DMSO_d6) δ=8.07 (d, J=2.0 Hz, 1H), 7.81-7.79 (m, 1H), 7.46 (d, J=8.0 Hz, 1H), 4.99-4.81 (m, 3H), 4.21-4.13 (m, 2H), 3.42-3.39 (m, 2H) ppm

### Step 3: Preparation of 1-imino-1-oxo-3,5-dihydro-2H-4,1λ6-benzoxathiepine-8-carboxylic acid

To a mixture of 8-bromo-1-imino-3,5-dihydro-2H-4,1λ6-benzoxathiepine 1-oxide (40 mg, 144.85 μmol) and diacetoxypalladium (3.25 mg, 14.48 μmol) in DMSO (3 mL) and H₂O (0.3 mL) was added K₂CO₃ (30.03 mg, 217.27 μmol) and dicyclohexyl(3-dicyclohexylphosphaniumylpropyl)phosphonium;ditetrafluoroborate (17.74 mg, 28.97 μmol). The mixture was degassed and purged with CO for 3 times and then was stirred at 100° C. for 4 h under CO atmosphere (15 psi). The mixture was poured into water (50 mL) and extracted with EA (20.0 mL×2), the combined organics were discarded. The aqueous was adjusted pH to 5 by HCl (1 M) and then was extracted with DCM (20.0 mL*3). The combined organic phase was washed with brine (50.0 mL*2), dried over Na₂SO₄, filtered and the filtrate was evaporated to dryness to give 1-imino-1-oxo-3,5-dihydro-2H-4,1λ6-benzoxathiepine-8-carboxylic acid (34 mg, crude) as a yellow solid.

### Example 2. N-[[2-[6-(azetidin-1-yl)-2-pyridyl]-1,6-naphthyridin-7-yl]methyl]-1,1-dioxo-3,5-dihydro-2H-4,1λ6-benzoxathiepine-8-carboxamide

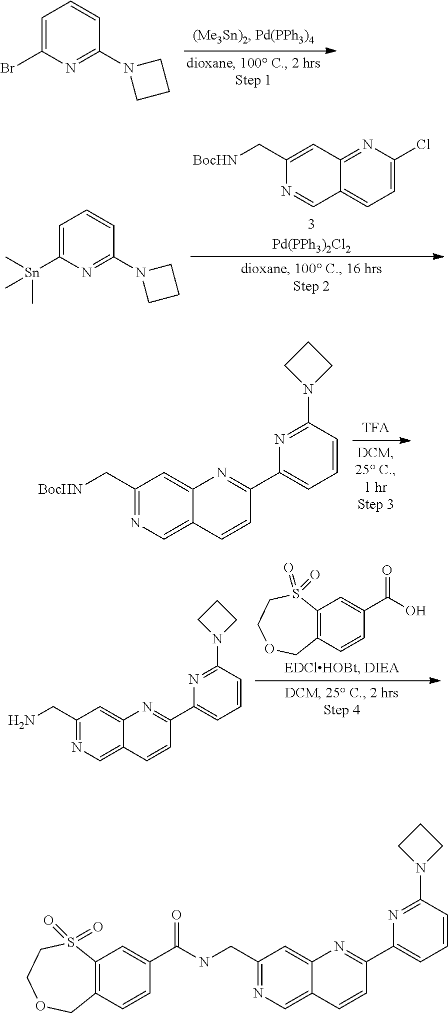

### Step 1. Preparation of [6-(azetidin-1-yl)-2-pyridyl] trimethyl-stannane

To a solution of 2-(azetidin-1-yl)-6-bromo-pyridine (150 mg, 703.98 μmol) in dioxane (3 mL) was added HEXAMETHYLDITIN (481.28 mg, 1.41 mmol) and Pd(PPh₃)₄ (81.35 mg, 70.40 μmol). The mixture was purged with N₂ 3× and then was stirred at 100° C. for 2 h under N₂ atmosphere. The reaction mixture was diluted with H₂O (200 mL) and extracted with EA (150 mL×3). The combined organic layers were washed with brine (200 mL), dried over Na₂SO₄, filtered and the filtrate was concentrated under reduced pressure to give [6-(azetidin-1-yl)-2-pyridyl]-trimethyl-stannane (209 mg, crude) as brown oil which was used into the next step without further purification. LCMS (ESI) m/z: [M+H]⁺=299.3.

### Step 2. Preparation of tort-butyl N-[[2-[6-(azetidin-1-yl)-2-pyridyl]-1,6-naphthyridin-7-yl]methyl]carbamate

To a solution of tert-butyl N-[(2-chloro-1,6-naphthyridin-7-yl)methyl]carbamate (100 mg, 340.43 μmol) in dioxane (2 mL) was added [6-(azetidin-1-yl)-2-pyridyl]-trimethyl-stannane (202.2 mg, 680.7 μmol μmol) and Pd(PPh₃)₂Cl₂ (23.9 mg, 34.04 μmol). The mixture was purged with N₂ for 3 times and then was stirred at 100° C. for 12 h under N₂ atmosphere. The reaction mixture was diluted with H₂O (20 mL) and extracted with EA (30 mL×3). The combined organic layers were washed with brine (30 mL), dried over Na₂SO₄, filtered and the filtrate was concentrated under reduced pressure to give a residue, which was purified by column chromatography (SiO₂, Petroleum ether/Ethyl acetate=10:1-1:1) to give left-butyl N-((2-(6-(azetidin-1-yl)pyridin-2-yl)-1,6-naphthyridin-7-yl)methyl)carbamate (70 mg, 173.45 μmol, 51% yield) as a yellow solid. LCMS (ESI) m/z: [M+H]⁺=392.4. ¹H NMR (400 MHz, CDCl₃) δ=9.22 (s, 1H), 8.68 (d, J=8.8 Hz, 1H), 8.33 (d, J=8.4 Hz, 1H), 7.98 (d, J=7.2 Hz, 1H), 7.93 (s, 1H), 7.72-7.61 (m, 1H), 6.43 (d, J=8.4 Hz, 1H), 4.88 (d, J=4.8 Hz, 2H), 4.18-4.14 (m, 4H), 2.18 (s, 2H), 1.50 (s, 9H) ppm.

### Step 3. Preparation of [2-(6-(azetidin-1-yl)-2-pyridyl]-1,6-naphthyridin-7-yl) methanamine

To a solution of tert-butyl N-((2-(6-(azetidin-1-yl)pyridin-2-yl)-1,6-naphthyridin-7-yl)methyl)carbamate (70 mg, 178.82 μmol) in DCM (3 mL) was added TFA (1 mL) at 0° C. The mixture was stirred at 25° C. for 2 h. The reaction mixture was poured into saturated aqueous NaHCO₃ (30 mL) and extracted with EA (30 mL×3). The combined organic layers were washed with brine (30 mL), dried over Na₂SO₄, filtered and the filtrate was concentrated under reduced pressure to give [2-[6-(azetidin-1-yl)-2-pyridyl]-1,6-naphthyridin-7-yl] methanamine (60 mg, crude) as a yellow solid, which was used into the next step without further purification.

LCMS (ESI) m/z: [M+H]⁺=292.4.

### Step 4. Preparation of N-[[2-[6-(azetidin-1-yl)-2-pyridyl]-1,6-naphthyridin-7-yl]methyl]-1,1-dioxo-3,5-dihydro-2H-4,16-benzoxathiepine-8-carboxamide (1)

To a solution of 1,1-dioxo-3,5-dihydro-2H-4,1λ6-benzoxathiepine-8-carboxylic acid (24.94 mg, 102.97 μmol) in DCM (1 mL) was added EDCl (21.38 mg, 111.55 μmol), HOBt (15.07 mg, 111.55 μmol) and DIEA (33.27 mg, 257.42 μmol). And then [2-[6-(azetidin-1-yl)-2-pyridyl]-1,6-naphthyridin-7-yl]methanamine (25 mg, 85.81 μmol) was added. The mixture was stirred at 25° C. for 2 h. The reaction mixture was diluted with H₂O (20 mL) and extracted with EA (30 mL*3). The combined organic layers were washed with brine (30 mL), dried over Na₂SO₄, filtered and the filtrate was concentrated under reduced pressure to give a residue, which was purified by prep-TLC (SiO₂, DCM:MeOH=15:1) to give the crude product. Then the crude product was further purified by Prep-HPLC (0.1% FA additive). The eluent was concentrated under reduced pressure to remove MeCN and the residue was lyophilized to give N-[[2-[6-(azetidin-1-yl)-2-pyridyl]-1,6-naphthyridin-7-yl]methyl]-1,1-dioxo-3,5-dihydro-2H-4,16-benzoxathiepine-8-carboxamide (10.21 mg, 19.21 μmol, 22% yield) as a yellow solid.

LCMS (ESI) m/z=[M+H]⁺=261.9.

¹H NMR (400 MHz, CD₃OD) δ=9.33 (s, 1H), 8.69-8.63 (m, 2H), 8.62-8.57 (m, 1H), 8.39 (s, 1H), 8.24 (d, J=2.0 Hz, 1H), 7.98 (s, 1H), 7.87 (d, J=7.2 Hz, 1H), 7.73-7.65 (m, 2H), 6.54 (d, J=7.6 Hz, 1H), 5.07 (s, 2H), 4.95 (s, 2H), 4.39-4.34 (m, 2H), 4.17-4.15 (m, 4H), 3.58-3.53 (m, 2H), 2.53-2.40 (m, 2H) ppm.

The following examples in Table 2 were prepared using standard chemical manipulations and procedures similar to those used for the preparation of Example 2.

TABLE 2
Compounds of the Invention

| | LCMS | |
|---|---|---|
| # | (m/z) | 1H NMR |
| 304 | 516.1 | ¹H NMR (400 MHz, CD₃OD) δ = 9.33 (s, 1H), 8.69-8.63 (m, 2H), 8.62-8.57 (m, 1H), |
| | | 8.39 (s, 1H), 8.24 (d, J = 2.0 Hz, 1H), 7.98 (s, 1H), 7.87 (d, J = 7.2 Hz, 1H), 7.73- |
| | | 7.65 (m, 2H), 6.54 (d, J = 7.6 Hz, 1H), 5.07 (s, 2H), 4.95 (s, 2H), 4.39-4.34 (m, 2H), |
| | | 4.17-4.15 (m, 4H), 3.58-3.53 (m, 2H), 2.53-2.40 (m, 2H) ppm. |
| 303 | 504.2 | ¹H NMR (400 MHz, CD₃OD) δ = 9.30 (s, 1H), 8.71 (d, J = 8.8 Hz, 1H), 8.62 (d, J = 1.6 |
| | | Hz, 1H), 8.57 (d, J = 8.4 Hz, 1H), 8.49 (d, J = 2.4 Hz, 1H), 8.22 (d, J = 1.6 Hz, 1H), |
| | | 7.96 (s, 1H), 7.82 (d, J = 7.2 Hz, 1H), 7.73-7.61 (m, 2H), 6.78 (d, J = 8.4 Hz, 1H), |
| | | 5.05 (s, 2H), 4.93 (s, 2H), 4.38-4.29 (m, 2H), 3.57-3.49(m, 2H), 3.19 (s, 6H) ppm. |
| 155 | 588.2 | ¹H NMR (400 MHz, CDCl₃) δ = 9.28 (s, 1H), 8.69 (d, J = 8.8 Hz, 1H), 8.55 (d, J = 1.6 |
| | | Hz, 1H), 8.40 (d, J = 8.8 Hz, 1H), 8.21-8.19 (m, 1H), 8.06 (d, J = 7.6 Hz, 2H), 7.84- |
| | | 7.77 (m, 1H), 7.72-7.70 (m, 1H), 7.49 (d, J = 7.6 Hz, 1H), 6.80 (d, J = 8.4 Hz, 1H), |
| | | 5.02 (d, J = 5.2 Hz, 4H), 4.40 (d, J = 12.8 Hz, 1H), 4.24-4.20 (m, 2H), 4.13-4.08 |
| | | (m, 1H), 3.80-3.79 (m, 2H), 3.40-3.28 (m, 1H), 2.67-2.61 (m, 2H), 1.35-1.32 (m, |
| | | 9H) ppm. |
| 159 | 588.2 | ¹H NMR (400 MHz, DMSO-d6) δ = 9.66-9.59 (m, 1H), 9.40 (s, 1H), 8.71-8.60 (m, |
| | | 2H), 8.53 (d, J = 2.0 Hz, 1H), 8.28-8.26 (m, 1H), 7.92 (d, J = 7.6 Hz, 1H), 7.83 (s, |
| | | 1H), 7.76-7.72 (m, 2H), 7.03 (d, J = 8.4 Hz, 1H), 4.97 (s, 2H), 4.82 (d, J = 5.2 Hz, |
| | | 2H), 4.38-4.24 (m, 3H), 4.04-3.99 (m, 1H), 3.74-3.60 (m, 3H), 2.52-2.52 (m, 2H), |
| | | 1.21 (d, J = 6.0 Hz, 6H), 1.19-1.14 (m, 3H) ppm. |
| 282 | 574.2 | ¹H NMR (400 MHz, DMSO-d6) δ = 9.65-9.63 (m, 1H), 9.41 (s, 1H), 8.66-8.64 (m, |
| | | 2H), 8.55 (d, J = 2.0 Hz, 1H), 8.28-8.26 (m, 1H), 7.91 (d, J = 7.2 Hz, 1H), 7.81 (s, |
| | | 1H), 7.75-7.72 (m, 2H), 7.03 (d, J = 8.8 Hz, 1H), 4.99 (s, 2H), 4.83 (d, J = 5.6 Hz, |
| | | 2H), 4.32 (br d, J = 11.6 Hz, 2H), 4.25-4.22 (m, 2H), 3.70-3.65 (m, 4H), 2.53 (br d, |
| | | J = 1.6 Hz, 2H), 1.22 (d, J = 6.0 Hz, 6H) ppm |
| 190 | 516.2 | ¹H NMR (400 MHz, DMSO-d₆) δ = 9.66-9.59 (m, 1H), 9.36 (s, 1H), 8.68-8.58 (m, |
| | | 2H), 8.56-8.51 (m, 1H), 8.27-8.25 (m, 1H), 7.80-7.71 (m, 3H), 7.42 (d, J = 7.2 Hz, |
| | | 1H), 4.98 (s, 2H), 4.81 (d, J = 5.6 Hz, 2H), 4.32-4.18 (m, 2H), 3.71-3.66 (m, 2H), |
| | | 3.53-3.49 (m, 2H), 3.07-2.95 (m, 5H) ppm. |
| 176 | 516.2 | ¹H NMR (400 MHz, CD₃OD) δ = 9.19 (s, 1H), 8.62 (d, J = 1.6 Hz, 1H), 8.45 (d, J = |
| | | 4.0 Hz, 2H), 8.27-8.17 (m, 2H), 7.86 (s, 1H), 7.64 (d, J = 8.0 Hz, 1H), 6.80 (d, J = |
| | | 8.4 Hz, 1H), 5.05 (s, 2H), 4.89 (br s, 2H), 4.36-4.32 (m, 2H), 3.61-3.51 (m, 4H), |
| | | 3.20-3.11 (m, 2H), 2.89 (s, 3H) ppm. |
| 167 | 541.1 | ¹H NMR (400 MHz, DMSO-d₆) δ = 9.65-9.62 (m, 1H), 9.43 (s, 1H), 8.76-8.70 (m, |
| | | 1H), 8.69-8.61 (m, 1H), 8.54 (d, J = 1.6 Hz, 1H), 8.32-8.23 (m, 2H), 8.02-7.94 (m, |
| | | 1H), 7.84 (s, 1H), 7.74 (d, J = 7.6 Hz, 1H), 7.12-7.16 (m, 1H), 6.65-6.33 (m, 1H), |
| | | 4.98 (s, 2H), 4.88-4.67 (m, 4H), 4.24-4.21 (m, 2H), 3.76-3.62 (m, 2H) ppm. |
| 165 | 519.2 | ¹H NMR (400 MHz, DMSO-d6) δ = 9.74-9.58 (m, 1H), 9.42 (s, 1H), 8.71 (d, J = 8.4 |
| | | Hz, 1H), 8.60 (d, J = 8.4 Hz, 1H), 8.54 (d, J = 1.6 Hz, 1H), 8.30-8.23 (m, 1H), 8.17 |
| | | (d, J = 7.6 Hz, 1H), 7.92-7.8 5 (m, 1H), 7.83 (s, 1H), 7.74 (d, J = 7.6 Hz, 1H), 6.92 |
| | | (d, J = 8.4 Hz, 1H), 5.56-5.43 (m, 1H), 4.98 (s, 2H), 4.83 (d, J = 5.6 Hz, 2H), 4.29- |
| | | 4.18 (m, 2H), 3.76-3.63 (m, 2H), 1.39 (d, J = 6.0 Hz, 6H) ppm |
| 240 | 592.3 | ¹H NMR (400 MHz, DMSO-d₆) δ = 9.70 (t, J = 5.6 Hz, 1H), 9.39 (s, 1H), 8.68-8.61 |
| | | (m, 2H), 8.46-8.40 (m, 1H), 8.18-8.16 (m, 1H), 7.91 (d, J = 7.6 Hz, 1H), 7.83 (s, |
| | | 1H), 7.75 (t, J = 7.6 Hz, 1H), 7.03 (d, J = 8.8 Hz, 1H), 5.02 (s, 2H), 4.82-4.81 (m, |
| | | 2H), 4.32-4.21 (m, 4H), 3.77-3.75 (m, 2H), 3.69-3.67 (m, 2H), 2.47 (br s, 2H), |
| | | 1.21 (d, J = 6.4 Hz, 6H) ppm. |
| 143 | 608.1 | ¹H NMR (400 MHz, DMSO-d₆) δ = 9.75-9.68 (m, 1H), 9.40 (s, 1H), 8.68-8.61 (m, 2H), |
| | | 8.51 (d, J = 1.6 Hz, 1H), 8.49 (s, 1H), 8.41 (d, J = 1.6 Hz, 1H), 7.92-7.84 (m, 2H), |
| | | 7.77-7.73 (m, 1H), 7.03 (d, J = 8.4 Hz, 1H), 5.20 (s, 2H), 4.81 (d, J = 5.6 Hz, 2H), |
| | | 4.32-4.22 (m, 4H), 3.81-3.70 (m, 2H), 3.68-3.61 (m, 2H), 2.54-2.52 (m, 2H), |
| | | 1.21 (d, J = 5.6 Hz, 6H) ppm. |
| 284 | 522.2 | ¹H NMR (400 MHz, DMSO-d₆) δ = 9.73-9.69 (m, 1H), 9.39 (s, 1H), 8.65 (s, 2H), |
| | | 8.41 (d, J = 1.2 Hz, 1H), 8.228.14 (m, 1H), 7.84-7.81 (m, 2H), 7.72-7.68 (m, 1H), |
| | | 6.82 (d, J = 8.4 Hz, 1H), 5.02 (s, 2H), 4.82 (d, J = 5.6 Hz, 2H), 4.24-4.21 (m, 2H), |
| | | 3.78-3.75 (m, 2H), 3.15 (s, 6H) ppm |
| 212 | 588.3 | ¹H NMR (400 MHz, DMSO-d₆) δ = 9.64 (s, 1H), 9.39 (s, 1H), 8.67-8.59 (m, 2H), |
| | | 8.55 (d, J = 2.0 Hz, 1H), 8.28-8.26 (m, 1H), 7.82-7.72 (m, 3H), 6.90-6.84 (m, 1H), |
| | | 4.99 (s, 2H), 4.82 (d, J = 5.6 Hz, 2H), 4.35-4.26 (m, 2H), 4.26-4.21 (m, 2H), 3.71- |
| | | 3.63 (m, 4H), 2.52-2.47 (m, 2H), 2.34 (s, 3H), 1.21 (d, J = 6.0 Hz, 6H) ppm. |
| 175 | 574.0 | ¹H NMR (400 MHz, MeOD) δ = 9.34-9.30 (m, 1H), 8.69-8.64 (m, 1H), 8.63-8.57 |
| | | (m, 2H), 8.54-8.50 (m, 1H), 8.23-8.18 (m, 1H), 7.98-7.95 (m, 1H), 7.95-7.91 (m, |
| | | 1H), 7.78-7.72 (m, 1H), 7.62-7.58 (m, 1H), 7.00-6.95 (m, 1H), 4.954.93 (m, 2H), |
| | | 4.86-4.84 (m, 2H), 4.37-4.29 (m, 2H), 4.16-3.98 (m, 2H), 3.85-3.74 (m, 2H), |
| | | 3.62-3.50 (m, 2H), 2.64-2.52 (m, 2H), 1.31-1.29 (m, 6H) ppm |
| 210 | 515.2 | ¹H NMR (400 MHz, DMSO-d6) δ = 9.65-9.62 (m, 1H), 9.41 (s, 1H), 8.73-8.68 (m, 1H), |
| | | 8.63-8.61 (m, 1H), 8.54 (d, J = 1.6 Hz, 1H), 8.46 (br s, 1H), 8.35 (d, J = 7.2 Hz, |
| | | 1H), 8.30-8.26 (m, 1H), 7.89 (t, J = 8.0 Hz, 1H), 7.83 (s, 1H), 7.74 (d, J = 7.6 Hz, |
| | | 1H), 7.51 (d, J = 7.6 Hz, 1H), 4.98 (s, 2H), 4.83 (d, J = 6.0 Hz, 2H), 4.24-4.21 (m, |
| | | 2H), 3.69-3.67 (m, 2H), 1.57 (s, 3H), 1.35-1.33 (m, 2H), 0.92-0.90 (m, 2H) ppm |
| 208 | 551.2 | ¹H NMR (400 MHz, DMSO-d6) δ = 9.69-9.58 (m, 1H), 9.46-9.41 (m, 1H), 8.75- |
| | | 8.66 (m, 2H), 8.58-8.52 (m, 1H), 8.48-8.46 (m, 1H), 8.31-8.24 (m, 1H), 8.04-7.93 |
| | | (m, 1H), 7.90-7.82 (m, 1H), 7.78-7.71 (m, 1H), 7.67-7.54 (m, 1H), 6.79-6.47(m, |
| | | 1H), 5.03-4.96 (m, 2H), 4.86-4.80 (m, 2H), 4.28-4.20 (m, 2H), 3.72-3.65 (m, 2H), |
| | | 1.51-1.43 (m, 2H), 1.38-1.31 (m, 2H) ppm. ¹⁹F NMR (400 MHz, DMSO-d₆) δ = −118.667 ppm. |
| 204 | 502.1 | ¹H NMR (400 MHz, DMSO-d₆) δ = 9.84-9.81 (m, 1H), 9.42 (s, 1H), 9.29 (d, J = 2.0 |
| | | Hz, 1H), 8.81 (d, J = 2.0 Hz, 1H), 8.72-8.66 (m, 1H), 8.64-8.60 (m, 1H), 8.34 (d, |
| | | J = 7.6 Hz, 1H), 7.90-7.82 (m, 2H), 7.47 (d, J = 7.6 Hz, 1H), 5.07 (s, 2H), 4.85 (d, J = |
| | | 5.6 Hz, 2H), 4.35-4.23 (m, 2H), 3.90-3.82 (m, 2H), 2.28-2.19 (m, 1H), 1.15-1.01 |
| | | (m, 4H) ppm. |
| 285 | 527.2 | ¹H NMR (400 MHz, DMSO-d₆) δ = 9.66-9.63 (m, 1H), 9.44 (s, 1H), 8.77-8.75 (m, |
| | | 1H), 8.66 (d, J = 8.8 Hz, 1H), 8.54 (d, J = 1.6 Hz, 1H), 8.46 (d, J = 7.6 Hz, 1H), 7.89 |
| | | (s, 3H), 7.85 (s, 1H), 7.74 (d, J = 8.0 Hz, 1H), 7.27 (d, J = 8.0 Hz, 1H), 4.98 (s, 2H), |
| | | 4.83 (d, J = 6.0 Hz, 2H), 4.24-4.22 (m, 2H), 3.69-3.67 (m, 2H) ppm. |
| 291 | 510.2 | ¹H NMR (400 MHz, DMSO-d₆) δ = 9.69-9.56 (m, 1H), 9.39 (s, 1H), 8.66 (s, 2H), |
| | | 8.54 (d, J = 1.6 Hz, 1H), 8.43 (s, 1H), 8.31-8.22 (m, 1H), 7.88-7.79 (m, 2H), 7.77- |
| | | 7.62 (m, 2H), 6.81 (d, J = 8.0 Hz, 1H), 4.98 (s, 2H), 4.82 (d, J = 5.6 Hz, 2H), 4.29- |
| | | 4.16 (m, 2H), 3.75-3.61 (m, 2H) ppm. |
| 276 | 558.2 | ¹H NMR (400 MHz, DMSO-d6) δ = 9.64 (br t, J = 5.6 Hz, 1H), 9.39 (s, 1H), 8.70- |
| | | 8.67 (m, 2H), 8.54 (d, J = 1.6 Hz, 1H), 8.27-8.25 (m, 1H), 7.92 (d, J = 7.6 Hz, 1H), |
| | | 7.81-7.73 (m, 3H), 6.82 (d, J = 8.8 Hz, 1H), 4.98 (s, 2H), 4.82-4.76 (m, 4H), 4.24- |
| | | 4.21 (m, 2H), 3.88 (br d, J = 12.0 Hz, 2H), 3.70-3.67 (m, 4H), 3.19-3.14 (m, 1H), |
| | | 1.94 (d, J = 8.4 Hz, 1H) ppm. |
| 274 | 490.1 | ¹H NMR (400 MHz, DMSO-d₆) δ = 9.64-9.61 (m, 1H), 9.37 (s, 1H), 8.65 (s, 2H), |
| | | 8.54 (d, J = 2.0 Hz, 1H), 8.27-8.25 (m, 1H), 7.79 (s, 1H), 7.76-7.73 (m, 2H), 7.58- |
| | | 7.54 (m, 1H), 6.75-6.73 (m, 1H), 6.62 (d, J = 8 .0 Hz, 1H), 4.98 (s, 2H), 4.82 (d, J = |
| | | 5.6 Hz, 2H), 4.24-4.22 (m, 2H), 3.69-3.67 (m, 2H), 2.92 (d, J = 4.8 Hz, 3H) ppm |
| 264 | 501.2 | ¹H NMR (400 MHz, DMSO-d₆) δ = 9.65-9.62 (m, 1H), 9.41 (s, 1H), 8.69-8.67 (m, |
| | | 1H), 8.62-8.60 (m, 1H), 8.54 (d, J = 1.6 Hz, 1H), 8.48 (br d, J = 3.2 Hz, 1H), 8.34 (d, |
| | | J = 7.2 Hz, 1H), 8.27-8.25 (m, 1H), 7.86-7.82 (m, 2H), 7.74 (d, J = 8.0 Hz, 1H), 7.47 |
| | | (d, J = 7.2 Hz, 1H), 4.98 (s, 2H), 4.82 (d, J = 5.6 Hz, 2H), 4.24-4.21 (m, 2H), 3.69- |
| | | 3.67 (m, 2H), 2.26-2.20 (m, 1H), 1.12-1.04 (m, 4H) ppm. |
| 289 | 503.3 | ¹H NMR (400 MHz, DMSO-d6) δ = 9.65 (s, 1H), 9.42 (s, 1H), 8.76-8.68 (m, 2H), |
| | | 8.47 (d, J = 1.2 Hz, 1H), 8.43-8.41 (m, 1H), 8.27 (d, J = 1.6 Hz, 1H), 7.92-7.84(m, |
| | | 1H), 7.84 (s, 1H), 7.74 (d, J = 7.6 Hz, 1H), 7.46 (d, J = 7.2 Hz, 1H), 4.98 (s, 2H), 4.83 |
| | | (d, J = 5.6 Hz, 2H), 4.27-4.19 (m, 2H), 3.75-3.64 (m, 2H), 3.22-3.12 (m, 1H), 1.35 |
| | | (d, J = 6.8 Hz, 6H) ppm. |
| 281 | 525.2 | ¹H NMR (400 MHz, CD₃OD) δ = 9.35 (s, 1H), 8.77-8.73 (m, 2H), 8.66-8.62 (m, |
| | | 2H), 8.23-8.21 (m, 1H), 8.14-8.10 (m, 1H), 8.00 (s, 1H), 7.84-7.82 (m, 1H), 7.64 |
| | | (d, J = 7.6 Hz, 1H), 5.05 (s, 2H), 4.94 (s, 2H), 4.35-4.33 (m, 2H), 3.54-3.52 (m, |
| | | 2H), 2.18-2.08 (m, 3H) ppm |
| 280 | 521.2 | ¹H NMR (400 MHz, CD₃OD) δ = 9.44 (s, 1H), 8.82 (d, J = 8.8 Hz, 1H), 8.69 (d, J = |
| | | 8.8 Hz, 1H), 8.65-8.56 (m, 2H), 8.23-8.21 (m, 1H), 8.10 (s, 1H), 8.04-8.00 (m, |
| | | 1H), 7.74-7.72 (m, 1H), 7.65 (d, J = 7.6 Hz, 1H), 5.05 (s, 2H), 4.97 (s, 2H), 4.35- |
| | | 4.33 (m, 2H), 3.54-3.52 (m, 2H), 1.83-1.77 (m, 6H) ppm |
| 241 | 517.2 | ¹H NMR (400 MHz, DMSO) δ = 9.36-9.30 (m, 1H), 8.73 (d, J = 8.8 Hz, 1H), 8.66- |
| | | 8.59 (m, 2H), 8.53-8.45 (m, 1H), 8.28-8.20 (m, 2H), 7.98 (s, 1H), 7.92-7.84 (m, |
| | | 1H), 7.65 (d, J = 7.6 Hz, 1H), 6.98 (d, J = 8.4 Hz, 1H), 5.06-5.04 (m, 2H), 4.94 (s, |
| | | 2H), 4.45-4.38 (m, 1H), 4.37-4.32 (m, 2H), 3.56-3.50 (m, 2H), 0.92-0.76 (m, 4H) ppm. |
| 239 | 505.2 | ¹H NMR (400 MHz, CD₃OD) δ = 9.32 (s, 1H), 8.71-8.67 (m, 1H), 8.64-8.59 (m, |
| | | 2H), 8.26-8.18 (m, 2H), 7.97 (s, 1H), 7.85-7.81 (m, 1H), 7.65 (d, J = 7.6 Hz, 1H), |
| | | 6.90 (d, J = 8.0 Hz, 1H), 5.05 (s, 2H), 4.93 (s, 2H), 4.58-4.52 (m, 2H), 4.37-4.33 |
| | | (m, 2H), 3.57-3.51 (m, 2H), 1.48-1.45 (m, 3H) ppm. |
| 237 | 545.1 | ¹H NMR (400 MHz, DMSO-d₆) δ = 9.67-9.65 (m, 1H), 9.46 (s, 1H), 8.78 (d, J = 8.8 |
| | | Hz, 1H), 8.61 (d, J = 7.6 Hz, 1H), 8.54 (d, J = 2.0 Hz, 1H), 8.50 (d, J = 8.8 Hz, 1H), |
| | | 8.29-8.21 (m, 2H), 7.87 (s, 1H), 7.74 (d, J = 7.6 Hz, 1H), 7.48 (d, J = 7.6 Hz, 1H), |
| | | 4.98 (s, 2H), 4.84 (d, J = 5.6 Hz, 2H), 4.27-4.20 (m, 2H), 3.71-3.65 (m, 2H) ppm. |
| 183 | 543.2 | ¹H NMR (400 MHz, DMSO-d6) δ = 9.66-9.62 (m, 1H), 9.43 (s, 1H), 8.76-8.70 (m, |
| | | 2H), 8.54 (d, J = 1.6 Hz, 1H), 8.48 (d, J = 7.6 Hz, 1H), 8.28-8.25 (m, 1H), 8.01- |
| | | 7.97 (m, 1H), 7.85 (s, 1H), 7.74 (d, J = 8.0 Hz, 2H), 6.98 (s, 1H), 4.98 (s, 2H), 4.83 |
| | | (d, J = 5.6 Hz, 2H), 4.35-4.32 (m, 2H), 4.24-4.22 (m, 2H), 3.90-3.87 (m, 2H), 3.69- |
| | | 3.67 (m, 2H), 2.73-2.68 (m, 2H) ppm. |
| 169 | 566.2 | ¹H NMR (400 MHz, DMSO-d6) δ = 9.66 (s, 1H), 9.47 (s, 1H), 8.90 (d, J = 8.4 Hz, 1H), |
| | | 8.78 (d, J = 8.8 Hz, 1H), 8.66 (d, J = 7.6 Hz, 1H), 8.55 (d, J = 2.0 Hz, 1H), 8.37 (s, |
| | | 1H), 8.31-8.21 (m, 2H), 8.20-8.12 (m, 1H), 7.91 (s, 2H), 7.88 (s, 1H), 7.75 (d, J = |
| | | 7.6 Hz, 1H), 4.99 (s, 2H), 4.85 (d, J = 5.6 Hz, 2H), 4.28-4.19 (m, 2H), 3.71-3.67 |
| | | (m, 2H), 2.57 (s, 6H) ppm. |
| 213 | 566.2 | ¹H NMR (400 MHz, DMSO-d₆) δ = 9.67 (t, J = 6.0 Hz, 1H), 9.39 (s, 1H), 8.65-8.61 |
| | | (m, 3H), 8.30-9.29 (m, 1H), 7.94-7.89 (m, 2H), 7.81 (s, 1H), 7.74 (t, J = 8.0 Hz, |
| | | 1H), 7.03 (d, J = 8.8 Hz, 1H), 4.82 (d, J = 5.6 Hz, 2H), 4.31 (br d, J = 11.2 Hz, 2H), |
| | | 3.70-3.65 (m, 2H), 3.43 (s, 3H), 2.47 (br s, 2H), 1.21 (d, J = 6.4 Hz, 6H) ppm. |
| 206 | 582.2 | ¹H NMR (400 MHz, DMSO-d₆) δ = 9.75-9.74 (m, 1H), 9.40 (s, 1H), 8.68-8.61 (m, |
| | | 3H), 8.47 (d, J = 8.0 Hz, 1H), 8.11 (d, J = 8.4 Hz, 1H), 7.91 (d, J = 7.6 Hz, 1H), 7.84 |
| | | (s, 1H), 7.84-7.56 (m, 2H), 7.03 (d, J = 8.4 Hz, 1H), 4.84 (d, J = 5.6 Hz, 2H), 4.32 |
| | | (d, J = 12.4 Hz, 2H), 3.68-3.65 (m, 2H), 3.39 (s, 3H), 2.46 (s, 2H), 1.21 (d, J = 6.0 |
| | | Hz, 6H) ppm. |
| 195 | 546.2 | ¹H NMR (400 MHz, DMSO-d₆) δ = 9.54 (d, J = 6.0 Hz, 1H), 9.40 (s, 1H), 8.73-8.59 |
| | | (m, 2H), 8.51 (d, J = 2.0 Hz, 1H), 8.45 (s, 1H), 8.22-8.20 (m, 1H), 7.91 (d, J = 7.2 |
| | | Hz, 1H), 7.81 (s, 1H), 7.78-7.72 (m, 1H), 7.64 (d, J = 8.0 Hz, 1H), 7.04 (d, J = 8.4 |
| | | Hz, 1H), 4.82 (d, J = 6.0 Hz, 2H), 4.32 (d, J = 12.0 Hz, 2H), 3.73-3.64 (m, 2H), 3.29 |
| | | (s, 3H), 2.73 (s, 3H), 2.54-2.53 (m, 2H), 1.22 (d, J = 6.0 Hz, 6H) ppm. |
| 192 | 576.3 | ¹H NMR (400 MHz, DMSO-d6) δ = 9.60-9.56 (m, 1H), 9.40 (s, 1H), 8.70-8.60 (m, |
| | | 2H), 8.53 (d, J = 2.0 Hz, 1H), 8.33-8.30 (m, 1H), 7.91 (d, J = 7.2 Hz, 1H), 7.85 (d, |
| | | J = 8.0 Hz, 1H), 7.82 (s, 1H), 7.76-7.72 (m, 1H), 7.03 (d, J = 8.4 Hz, 1H), 4.90 (s, 2H), |
| | | 4.82 (d, J = 5.6 Hz, 2H), 4.31 (br d, J = 11.2 Hz, 2H), 3.73-3.63 (m, 2H), 3.43 (s, |
| | | 3H), 3.31-3.31 (m, 3H), 2.53 (br d, J = 2.0 Hz, 2H), 1.21 (d, J = 6.0 Hz, 6H) ppm. |
| 180 | 567.2 | ¹H NMR (400 MHz, DMSO-d6) δ = 9.70-9.66 (m, 1H), 9.48 (d, J = 0.4 Hz, 1H), 8.79- |
| | | 8.73 (m, 1H), 8.67 (d, J = 8.4 Hz, 1H), 8.63-8.57 (m, 2H), 8.32-8.29 (m, 1H), 7.93 |
| | | (d, J = 8.4 Hz, 1H), 7.88 (s, 1H), 7.71 (d, J = 4.8 Hz, 1H), 4.83 (d, J = 6.0 Hz, 2H), |
| | | 4.68 (br d, J = 12.4 Hz, 2H), 3.65-3.43 (m, 2H), 3.43 (s, 3H), 2.63 (br d, J = 2.4 Hz, |
| | | 2H), 1.20 (d, J = 6.0 Hz, 6H) ppm. |
| 157 | 583.4 | ¹H NMR (400 MHz, DMSO-d6) δ = 9.75-9.71 (m, 1H), 9.49 (s, 1H), 8.80-8.75 (m, |
| | | 1H), 8.68 (d, J = 8.8 Hz, 1H), 8.61 (d, J = 4.8 Hz, 2H), 8.48-8.45 (m, 1H), 8.11 (d, |
| | | J = 8.0 Hz, 1H), 7.93-7.55 (m, 3H), 4.86 (d, J = 6.0 Hz, 2H), 4.69 (d, J = 12.8 Hz, 2H), |
| | | 3.67-3.60 (m, 2H), 3.40 (s, 3H), 2.68-2.60 (m, 2H), 1.21 (d, J = 6.0 Hz, 6H) ppm. |
| 270 | 537.3 | ¹H NMR (400 MHz, MeOD) δ = 9.34 (s, 1H), 8.75-8.71 (m, 1H), 8.66-8.61 (m, 2H), |
| | | 8.55-8.50 (m, 1H), 8.24-8.21 (m, 1H), 7.99 (s, 1H), 7.96-7.91 (m, 1H), 7.65 (d, J = |
| | | 8.0 Hz, 1H), 7.52 (d, J = 7.6 Hz, 1H), 5.05 (s, 2H), 4.94 (s, 2H), 4.37-4.33 (m, 2H), |
| | | 3.55-3.52 (m, 2H), 3.21-3.14 (m, 1H), 2.49-2.42 (m, 1H), 2.00-1.90 (m, 1H) ppm. |
| | | Chiral SFC: AD-3-IPA + ACN(DEA)-40-3 ML-35T, Rt = 1.639 min, ee % = 100.0%. |
| 267 | 537.3 | ¹H NMR (400 MHz, MeOD) δ = 9.34 (s, 1H), 8.73 (d, J = 8.4 Hz, 1H), 8.65-8.60 (m, |
| | | 2H), 8.54-8.51 (m, 1H), 8.24-8.21 (m, 1H), 7.99 (s, 1H), 7.96-7.91 (m, 1H), 7.65 (d, |
| | | J = 8.0 Hz, 1H), 7.51 (d, J = 7.6 Hz, 1H), 5.05 (s, 2H), 4.94 (s, 2H), 4.38-4.31 (m, |
| | | 2H), 3.56-3.51 (m, 2H), 3.21-3.12 (m, 1H), 2.52-2.41 (m, 1H), 2.02-1.91 (m, 1H) ppm. |
| | | Chiral SFC: AD-3-IPA + ACN(DEA)-40-3 ML-35T, Rt = 2.008 min, ee % = 98.252 %. |
| 217 | 538.3 | ¹H NMR (400 MHz, MeOD) δ = 9.45-9.40 (m, 1H), 8.99-8.90 (m, 1H), 8.81-8.71 |
| | | (m, 2H), 8.66-8.63 (m, 1H), 8.56-8.49 (m, 1H), 8.27-8.21 (m, 1H), 8.06-8.01 (m, |
| | | 1H), 7.70-7.63 (m, 1H), 5.10-5.06 (m, 2H), 4.97 (s, 2H), 4.40-4.33(m, 2H), 3.58- |
| | | 3.52 (m, 2H), 3.30-3.25 (m, 1H), 2.64-2.52 (m, 1H), 2.15-2.00 (m, 1H) ppm. |
| | | SFC: OD-3-MeOH(DEA)-40-3 ML-35T.lcm, rt = 1.629, ee % = 100%. |
| 218 | 538.3 | ¹H NMR (400 MHz, MeOD) δ = 9.45-9.38 (m, 1H), 8.97-8.92 (m, 1H), 8.80-8.70 |
| | | (m, 2H), 8.66-8.62 (m, 1H), 8.54-8.50 (m, 1H), 8.46-8.40 (m, 1H), 8.26-8.21 (m, |
| | | 1H), 8.06-8.02 (m, 1H), 7.69-7.64 (m, 1H), 5.09-5.06 (m, 2H), 4.99-4.95 (m, 2H), |
| | | 4.40-4.34 (m, 2H), 3.58-3.52 (m, 2H), 3.31-3.25 (m, 1H), 2.64-2.53 (m, 1H), |
| | | 2.14-2.01 (m, 1H) ppm. |
| | | SFC: OD-3-MeOH(DEA)-40-3 ML-35T.lcm, rt = 2.531, ee % = 100%. |
| 219 | 551.1 | ¹H NMR (400 MHz, DMSO-d6) δ = 9.68-9.65 (m, 1H), 9.44 (s, 1H), 8.76-8.67 (m, |
| | | 2H), 8.56-8.50 (m, 2H), 8.28-8.25 (m, 1H), 8.05-8.00 (m, 1H), 7.85 (s, 1H), 7.76- |
| | | 7.70 (m, 2H), 4.98 (s, 2H), 4.83 (d, J = 5.6 Hz, 2H), 4.26-4.20 (m, 2H), 3.71-3.67 |
| | | (m, 2H), 2.65-2.58 (m, 1H), 1.81-1.73 (m, 1H), 1.69 (s, 3H) ppm. |
| | | Chiral SFC: OJ-3-MeOH(DEA)-40-3 ML-35T, Rt = 0.987 min, ee % = 100%. |
| 214 | 551.1 | ¹H NMR (400 MHz, DMSO-d6) δ = 9.66-9.63 (m, 1H), 9.44 (s, 1H), 8.76-8.67 (m, |
| | | 2H), 8.59-8.48 (m, 2H), 8.28-8.25 (m, 1H), 8.05-8.00 (m, 1H), 7.85 (s, 1H), 7.75- |
| | | 7.69 (m, 2H), 4.98 (s, 2H), 4.83 (d, J = 6.0 Hz, 2H), 4.29-4.18 (m, 2H), 3.73-3.64 |
| | | (m, 2H), 2.64-2.60 (m, 1H), 1.81-1.72 (m, 1H), 1.69 (s, 3H) ppm. |
| | | Chiral SFC: OJ-3-MeOH(DEA)-40-3 ML-35T, Rt = 1.176 min, ee % = 100%. |
| 37 | 592.5 | ¹H NMR (400 MHz, DMSO-d6) δ = ¹H NMR (400 MHz, DMSO-d6) δ = 9.66-9.63 |
| | | (m, 1H), 9.44 (s, 1H), 8.76-8.67 (m, 2H), 8.59-8.48 (m, 2H), 8.28-8.25 (m, 1H), |
| | | 8.05-8.00 (m, 1H), 7.85 (s, 1H), 7.75-7.69 (m, 2H), 4.98 (s, 2H), 4.83 (d, J = 6.0 |
| | | Hz, 2H), 4.29-4.18 (m, 2H), 3.73-3.64 (m, 2H), 2.64-2.60 (m, 1H), 1.81-1.72 (m, |
| | | 1H), 1.69 (s, 3H) ppm. |
| 15 | 588.5 | ¹H NMR (400 MHz, DMSO-d6) δ = 9.56-9.53 (m, 1H), 9.39 (s, 1H), 8.65-8.59 (m, |
| | | 2H), 8.48 (s, 1H), 8.28-8.25 (m, 1H), 7.90 (d, J = 7.4 Hz, 1H), 7.807.75 (m, 2H), 7.03 |
| | | (d, J = 8.4 Hz, 1H), 7.01 (s, 1H), 4.88 (d, J = 5.6 Hz, 2H), 4.41-4.28 (m, 4H), 3.69- |
| | | 3.64 (m, 3H), 2.54-2.52 (m, 2H), 2.32-2.30 (m, 1H), 2.20-2.16 (m, 1H), 1.34 (d, |
| | | J = 7.2 Hz, 3H ), 1.21 (d, J = 6.4 Hz, 6H) ppm. |
| 13 | 606.3 | ¹H NMR (400 MHz, DMSO-d6) δ = 9.70 (m, 1H), 9.40 (s, 1H), 8.65 (m, 2H), 8.40 (m, |
| | | 1H), 8.20 (m, 1H), 7.92 (d, J = 7.6 Hz, 1H), 7.85 (s, 1H), 7.78-7.70 (m, 1H), 7.03 (d, |
| | | J = 8.4 Hz, 1H), 5.12-5.03 (m, 1H), 5.01-4.91 (m, 1H), 4.82 (m, 2H), 4.36-4.25 |
| | | (m, 3H), 4.03 (m, 1H), 3.77-3.63 (m, 3H), 2.53-2.52 (m, 2H), 1.24-1.16 (m, 9H) ppm. |
| 101 | 588.3 | ¹H NMR (400 MHz, DMSO-d6) δ = 9.61-9.51 (m, 1H), 9.40 (s, 1H), 8.70-8.59 (m, |
| | | 2H), 8.40 (s, 1H), 8.16 (s, 1H), 7.91 (d, J = 7.4 Hz, 1H), 7.80 (s, 1H), 7.74 (m, 1H), |
| | | 7.03 (d, J = 8.6 Hz, 1H), 5.06 (s, 2H), 4.81 (br d, J = 5.6 Hz, 2H), 4.34-4.27 (m, 2H), |
| | | 4.25-4.17 (m, 2H), 3.73-3.62 (m, 4H), 2.53-2.52 (m, 2H), 2.48-2.47 (m, 3H), |
| | | 1.21 (d, J = 6.3 Hz, 6H) ppm. |
| 63 | 519.1 | ¹H NMR (400 MHz, DMSO-d6) δ = 9.72-9.69 (m, 1H), 9.41 (s, 1H), 8.71-8.66 (m, |
| | | 1H), 8.64-8.58 (m, 1H), 8.41 (s, 1H), 8.34 (d, J = 8.0 Hz, 1H), 8.18-8.16 (m, 1H), |
| | | 7.88-7.81 (m, 2H), 7.46 (d, J = 7.6 Hz, 1H), 5.03 (s, 2H), 4.83 (d, J = 5.6 Hz, 2H), |
| | | 4.28-4.19 (m, 2H), 3.80-3.72 (m, 2H), 2.28-2.18 (m, 1H), 1.16-1.00 (m, 4H) ppm. |

### Example 3. N-[[2-[6-(azetidin-1-yl)-2-pyridyl]-1,6-naphthyridin-7-yl]methyl]-1,1-dioxo-3,5-dihydro-2H-4,1λ6-benzoxathiepine-3-carboxamide

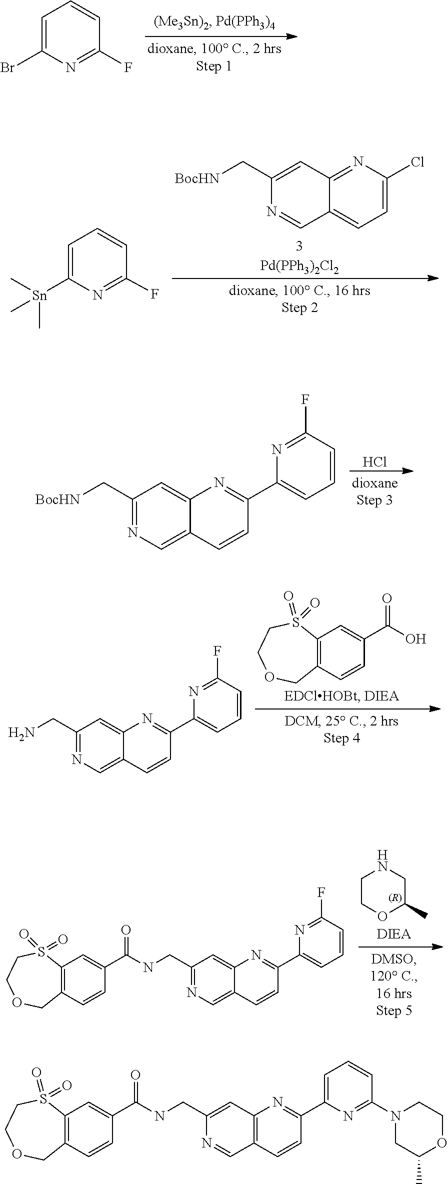

### Step 1. Preparation of (6-fluoro-2-pyridyl)-trimethyl-stannane

To a mixture of 2-bromo-6-fluoro-pyridine (500 mg, 2.84 mmol) in dioxane (5 mL) was added trimethyl(trimethylstannyl)stannane (2.79 g, 8.52 mmol) and Pd(PPh₃)₄ (328.31 mg, 284.11 μmol). The mixture was purged with N₂ for 1 min and then was stirred at 100° C. for 2 h. Water (20 mL) was added and the mixture was extracted with EtOAc (20 mL×2). The combined organic phase was washed with brine (30 mL), dried over anhydrous Na₂SO₄, filtered and the filtrate was concentrated under vacuum to give (6-fluoro-2-pyridyl)-trimethyl-stannane (730 mg, crude) as brown oil.

LCMS (ESI) m/z=[M+H]⁺=261.9.

### Step 2. Preparation of tert-butyl N-[[2-(6-fluoro-2-pyridyl)-1,6-naphthyridin-7-yl]methyl]carbamate

To a mixture of tert-butyl 11 [(2-chloro-1,6-naphthyridin-7-yl)methyl]carbamate (300 mg, 1.02 mmol) and (6-fluoro-2-pyridyl)-trimethyl-stannane (530.85 mg, 2.04 mmol) in dioxane (6 mL) was added Pd(PPh₃)₂Cl₂ (71.68 mg, 102.13 μmol) and the mixture was purged with N₂ for 1 min. The resulting mixture was stirred at 110° C. for 16 h. Then the reaction mixture was poured into Sat. KF (30 mL) and was stirred for 30 min and the mixture was extracted with EtOAc (30 mL×2). The combined organic phase was washed with brine (40 mL), dried over anhydrous Na₂SO₄, filtered and the filtrate was concentrated under vacuum. The reaction mixture was purified by column chromatography (SiO₂, PE:EtOAc=20:1-1:1) to give tert-butyl N-[[2-(6-fluoro-2-pyridyl)-1,8-naphthyridin-7-yl]methyl]carbamate (230 mg, 649.03 μmol, 64% yield) as a yellow solid.

LCMS (ESI) m/z: [M+H]⁺=355.1.

¹H NMR (400 MHz, CDCl₃) δ=9.26 (s, 1H), 8.63-8.58 (m, 2H), 8.41-8.39 (m, 1H), 8.04-7.98 (m, 1H), 7.94 (s, 1H), 7.09-7.06 (m, 1H), 5.49 (br s, 1H), 4.69 (br d, J=5.2 Hz, 2H), 1.50 (s, 9H) ppm.

### Step 3. Preparation of [2-(6-fluoro-2-pyridyl)-1,6-naphthyridin-7-yl]methanamine

A mixture of tert-butyl-N-[[2-(6-fluoro-2-pyridyl)-1,6-naphthyridin-7-yl]methyl]carbamate (220 mg, 620.81 μmol) in HCl/dioxane (2 mL) was stirred at 25° C. for 1 hr. The mixture was evaporated to dryness and the residue was triturated with MTBE (20 mL×2). The mixture was filtered and the fitter cake was evaporated to dryness to give [2-(6-fluoro-2-pyridyl)-1,6-naphthyridin-7-yl]methanamine (180 mg, crude, HCl) as a yellow solid.

LCMS (ESI) m/z: [M+H]⁺=255.1.

### Step 4. Preparation of N-[[2-(6-fluoro-2-pyridyl)-1,6-naphthyridin-7-yl]methyl]-1,1-dioxo-3,6-dihydro-2H-4,1 λ6-benzoxathiepine-8-carboxamide

To a mixture of [2-(6-fluoro-2-pyridyl)-1,6-naphthyridin-7-yl]methanamine (180 mg, 619.15 μmol) and 1,1-dioxo-3,5-dihydro-2H-4,1λ6-benzoxathiepine-8-carboxylic acid (180 mg, 0.743 mmol) in DCM (2 mL) was added DIEA (320.08 mg, 2.48 mmol), EDCl (178 mg, 0.928 mmol) and HOBt (125.49 mg, 928.72 μmol). The mixture was stirred at 25° C. for 1 hr. The mixture was poured into water (20 mL) and extracted with EA (10.0 mL×3). The combined organics were washed with brine (20.0 mL), dried over Na₂SO₄, filtered and the filtrate was evaporated to dryness. The residue was purified by prep-HPLC (0.1% FA condition) and the eluent was concentrated under vacuum to remove the MeCN. The residue was lyophilized to give N-[[2-(6-fluoro-2-pyridyl)-1,6-naphthyridin-7-yl]methyl]-1,1-dioxo-3,5-dihydro-2H-4,1λ6-benzoxathiepine-8-carboxamide (253 mg, 0.528 mmol, 85% yield) as a white solid.

LCMS (ESI) m/z: [M+H]⁺=479.0.

¹H NMR (400 MHz, DMSO-d₆) δ=9.68-9.81 (m, 1H), 9.45 (d, J=0.8 Hz, 1H), 8.75-8.72 (m, 1H), 8.54-8.49 (m, 3H), 8.26-8.26 (m, 1H), 8.25-8.17 (m, 1H), 7.84 (s, 1H), 7.73 (d, J=8.0 Hz, 1H), 7.39-7.36 (m, 1H), 4.97 (s, 2H), 4.83 (d, J=5.6 Hz, 2H), 4.23-4.21 (m, 2H), 3.68-3.66 (m, 2H) ppm.

### Step 5. Preparation of N-[[2-[6-[(2R)-2-methylmorpholin-4-yl]-2-pyridyl]-1,6-naphthyridin-7-yl]methyl]-1,1-dioxo-3,6-dihydro-2H-4,1λ6-benzoxathiepine-8-carboxamide (37)

To a mixture of N-[[2-(6-fluoro-2-pyridyl)-1,6-naphthyridin-7-yl]methyl]-1,1-dioxo-3,5-dihydro-2H-4,1λ6-benzoxathiepine-8-carboxamide (20 mg, 0.0418 mmol) and (2R)-2-methyl morpholine;hydrochloride (17.26 mg, 125.39 μmol) in DMSO (1 mL) was added DIEA (27.0 mg, 0.209 mmol). The mixture was stirred at 120° C. for 16 h. Then the mixture was poured into Sat·NaHCO₃ (20 mL) and was extracted with EA (10.0 mL×3). The combined organics were washed with brine (20.0 mL), dried over Na₂SO₄, filtered and the filtrate was evaporated to dryness. The residue was purified by prep-HPLC (column: Shim-pack C18 150*25*10 um;mobile phase: [water (0.225% FA)-ACN];B %: 38% 58%, 10 min) and the eluent was concentrated under vacuum to remove the MeCN. The residue was lyophilized to give N-[[2-[6-[(2R)-2-methylmorpholin-4-yl]-2-pyridyl]-1,6-naphthyridin-7-yl]methyl]-1,1-dioxo-3,5-dihydro-2H-4,1λ6-benzoxathiepine-8-carboxamide (15.89 mg, 28.24 μmol, 83% yield, FA) as a yellow solid.

LCMS (ESI) m/z: [M+H]⁺=580.3.

¹H NMR (400 MHz, DMSO-d₆) δ=9.65-9.62 (m, 1H), 9.40 (s, 1H), 8.68-8.61 (m, 2H), 8.54 (d, J=2.0 Hz, 1H), 8.46 (s, 1H), 8.28-8.25 (m, 1H), 7.92 (d, J=7.2 Hz, 1H), 7.81 (s, 1H), 7.77-7.73 (m, 2H), 7.03 (d, J=8.4 Hz, 1H), 4.98 (s, 2H), 4.82 (d, J=5.6 Hz, 2H), 4.30-4.22 (m, 4H), 3.99-3.96 (m, 1H), 3.70-3.58 (m, 4H), 2.94-2.87 (m, 1H), 2.82-2.58 (m, 1H), 1.22 (d, J=8.0 Hz, 3H) ppm.

The following examples in Table 3 were prepared using standard chemical manipulations and procedures similar to those used for the preparation of Example 3.

TABLE 3
Compounds of the Invention

| | LCMS | |
|---|---|---|
| # | (m/z) | 1H NMR |
| 225 | 560.3 | ¹H NMR (400 MHz, DMSO-d₆) δ = 9.65-9.62 (m, 1H), 9.40 (s, 1H), 8.68-8.61 (m, 2H), |
| | | 8.54 (d, J = 2.0 Hz, 1H), 8.46 (s, 1H), 8.28-8.25 (m, 1H), 7.92 (d, J = 7.2 Hz, 1H), 7.81 |
| | | (s, 1H), 7.77-7.73 (m, 2H), 7.03 (d, J = 8.4 Hz, 1H), 4.98 (s, 2H), 4.82 (d, J = 5.6 Hz, |
| | | 2H), 4.30-4.22 (m, 4H), 3.99-3.96 (m, 1H), 3.70-3.58 (m, 4H), 2.94-2.87 (m, 1H), |
| | | 2.62-2.56 (m, 1H), 1.22 (d, J = 6.0 Hz, 3H) ppm. |
| 224 | 548.3 | ¹H NMR (400 MHz, DMSO-d₆) δ = 9.64-9, 61 (m, 1H), 9.39 (s, 1H), 8.67 (s, 2H), 8.54 |
| | | (d, J = 1.6 Hz, 1H), 8.43 (s, 1H), 8.27-8.25 (m, 1H), 7.85 (d, J = 7.2 Hz, 1H), 7.81 (s, |
| | | 1H), 7.75-7.69 (m, 2H), 6.69 (d, J = 8.0 Hz, 1H), 5.56-5.42 (m, 1H), 4.98 (s, 2H), |
| | | 4.82 (d, J = 5.6 Hz, 2H), 4.24-4.21 (m, 2H), 3.93-3.74 (m, 3H), 3.69-3.67 (m, 2H), |
| | | 3.58-3.51 (m, 1H), 2.53 (br s, 1H), 2.28-2.16 (m, 1H) ppm. |
| 223 | 560.2 | ¹H NMR (400 MHz, MeOD) δ = 9.33 (s, 1H), 8.72-8.56 (m, 3H), 8.23-8.20 (m, 1H), |
| | | 7.99 (s, 1H), 7.94 (d, J = 7.6 Hz, 1H), 7.80-7.71 (m, 1H), 7.64 (d, J = 7.6 Hz, 1H), |
| | | 6.97 (d, J = 8.4 Hz, 1H), 5.05 (s, 2H), 4.94 (s, 2H), 4.40-4.20 (m, 4H), 4.06-4.02 (m, |
| | | 1H), 3.81-3.67 (m, 2H), 3.57-3.50 (m, 2H), 3.03-2.95 (m, 1H), 2.70-2.61 (m, 1H), |
| | | 1.28 (d, J = 6.4 Hz, 3H) ppm. |
| 222 | 548.2 | ¹H NMR (400 MHz, DMSO-d₆) δ = 9.65-9.62 (m, 1H), 9.39 (s, 1H), 8.67 (s, 2H), 8.54 |
| | | (d, J = 1.6 Hz, 1H), 8.36 (br s, 1H), 8.28-8.25 (m, 1H), 7.89-7.78 (m, 2H), 7.77-7.65 |
| | | (m, 2H), 6.69 (d, J = 8.4 Hz, 1H), 5.60-5.40 (m, 1H), 4.98 (s, 2H), 4.82 (br d, J = 5.6 |
| | | Hz, 2H), 4.28-4.17 (m, 2H), 3.94-3.83 (m, 1H), 3.81-3.65 (m, 4H), 3.58-3.51 (m, |
| | | 1H), 2.54 (br s, 1H), 2.29-2.13 (m, 1H) ppm. |
| 221 | 560.3 | ¹H NMR (400 MHz, DMSO-d6) δ = 9.69-9.57 (m, 1H), 9.39 (s, 1H), 8.69-8.63 (m, |
| | | 1H), 8.62-8.58 (m, 1H), 8.54 (s, 1H), 8.30-8.22 (m, 1H), 7.87 (d, J = 7.6 Hz, 1H), |
| | | 7.80 (s, 1H), 7.77-7.65 (m, 2H), 6.54 (d, J = 8.4 Hz, 1H), 4.98 (s, 2H), 4.82-4.81(m, |
| | | 2H), 4.29-4.18 (m, 2H), 4.11 (s, 2H), 3.85-3.75 (m, 2H), 3.72-3.65 (m, 2H), 3.57- |
| | | 3.56 (m, 2H), 3.30 (s, 3H), 3.03-2.95 (m, 1H) ppm. |
| 220 | 530.3 | ¹H NMR (400 MHz, DMSO-d6) δ = 9.64-9.63 (m, 1H), 9.38 (s, 1H), 8.65 (s, 2H), 8.54 |
| | | (d, J = 1.6 Hz, 1H), 8.27-8.25 (m, 1H), 7.83-7.77 (m, 2H), 7.74 (d, J = 7.6 Hz, 1H), |
| | | 7.67 (d, J = 8.4 Hz, 1H), 6.61 (d, J = 8.4 Hz, 1H), 4.98 (s, 2H), 4.82 (d, J = 5.6 Hz, 2H), |
| | | 4.29-4.17 (m, 2H), 3.71-3.65 (m, 2H), 3.53-3.51 (m, 4H), 2.05-1.94 (m, 4H) ppm. |
| 216 | 574.2 | ¹H NMR(400 MHz, DMSO-d₆) δ = 9.64-9.61 (m, 1H), 9.39 (s, 1H), 8.67-8.60 (m, 2H), |
| | | 8.54 (d, J = 1.6 Hz, 1H), 8.43(s, 1H), 8.27-8.25 (m, 1H), 7.89-7.79 (m, 4H), 7.01 (d, |
| | | J = 8.8 Hz, 1H), 4.98 (s, 2H), 4.81 (d, J = 5.4 Hz, 2H), 4.24-4.21 (m, 2H), 4.10-4.06 (m, |
| | | 2H), 3.75-3.66 (m, 4H), 3.38-3.37 (m, 2H), 1.25-1.18 (m, 6H)ppm |
| | | Chiral SFC: OJ-3-MeOH(DEA)-40-3 mL-35T.lcm, Rt = 1.462 mins, ee % = 100%. |
| 215 | 574.4 | ¹H NMR (400 MHz, DMSO-d6) δ = 9.64-9.61 (m, 1H), 9.39 (s, 1H), 8.69-8.59 (m, |
| | | 2H), 8.54 (d, J = 1.6 Hz, 1H), 8.45 (s, 1H), 8.27-8.25 (m, 1H), 7.88 (d, J = 7.6 Hz, |
| | | 1H), 7.81 (s, 1H), 7.74-7.70 (m, 2H), 7.01 (d, J = 8.4 Hz, 1H), 4.98 (s, 2H), 4.82 (d, |
| | | J = 6.0 Hz, 2H), 4.24-4.20 (m, 2H), 4.10-4.06 (m, 2H), 3.76-3.67 (m, 4H), 3.38 (s, |
| | | 2H), 1.21 (d, J = 6.4 Hz, 6H) ppm |
| | | Chiral SFC: IC-3-MeOH + ACN(DEA)-50-5 min-3 mL-35T. lcm; RT = 3.169 min; |
| | | ee % = 100%. |
| 202 | 566.2 | ¹H NMR (400 MHz, DMSO-d₆) δ = 9.71-9.56 (m, 1H), 9.40 (s, 1H), 8.67 (s, 2H), 8.54 |
| | | (d, J = 1.6 Hz, 1H), 8.31-8.19 (m, 1H), 7.92 (d, J = 7.2 Hz, 1H), 7.81 (s, 1H), 7.80- |
| | | 7.71 (m, 2H), 6.74 (d, J = 8.4 Hz, 1H), 4.98 (s, 2H), 4.82 (br d, J = 5.6 Hz, 2H), 4.28- |
| | | 4.18 (m, 2H), 4.09-3.95 (m, 2H), 3.80-3.72 (m, 2H), 3.71-3.61 (m, 2H), 2.68-2.55 |
| | | (m, 2H) ppm. |
| 201 | 560.3 | ¹H NMR (400 MHz, DMSO) δ = 9.66-9.56 (m, 1H), 9.41-9.34 (m, 1H), 8.66 (s, 2H), |
| | | 8.54 (d, J = 1.6 Hz, 1H), 8.29-8.24 (m, 1H), 8.14-8.12 (m, 1H), 7.83-7.79 (m, 2H), |
| | | 7.74 (d, J = 7.6 Hz, 1H), 7.71-7.65 (m, 1H), 6.63 (d, J = 8.4 Hz, 1H), 4.98 (s, 2H), |
| | | 4.82 (d, J = 5.6 Hz, 2H), 4.29-4.20 (m, 2H), 4.16-4.09 (m, 1H), 3.72-3.66 (m, 2H), |
| | | 3.65-3.59 (m, 3H), 3.54-3.46 (m, 1H), 3.30 (s, 3H), 2.15-2.06 (m, 2H) ppm. |
| | | SFC: AD-3-EtOH (DEA)-60-3 mL-5 min-35T.lcm, Rt = 3.999 mins, ee % value = 100%. |
| 200 | 560.2 | ¹H NMR (400 MHz, DMSO-d6) δ = 9.64-9.61 (m, 1H), 9.38 (s, 1H), 8.65 (s, 2H), 8.53 |
| | | (d, J = 1.6 Hz, 1H), 8.27-8.25 (m, 1H), 7.82-7.80 (m, 2H), 7.74 (d, J = 7.6 Hz, 1H), |
| | | 7.69-7.67 (m, 1H), 6.63 (d, J = 8.0 Hz, 1H), 4.98 (s, 2H), 4.82 (d, J = 5.6 Hz, 2H), 4.24- |
| | | 4.21 (m, 2H), 4.12-4.10 (m, 1H), 3.72-3.66 (m, 2H), 3.69-3.62(m, 3H), 3.50 (d, |
| | | J = 9.6 Hz, 1H), 3.29 (s, 3H), 2.13-2.08 (m, 2H) ppm. |
| | | Chiral SFC: AD-3-EtOH (DEA)-60-3 mL-5 min-35T.lcm. Rt = 3.435 min, ee % = 100%. |
| 308 | 580.2 | ¹H NMR (400 MHz, DMSO-d₆) δ = 9.71-9.58 (m, 1H), 9.40 (d, J = 0.8 Hz, 1H), 8.73- |
| | | 8.65 (m, 1H), 8.64-8.58 (m, 1H), 8.54 (d, J = 2.4 Hz, 1H), 8.43 (s, 1H), 8.31-8.22 (m, |
| | | 1H), 7.91 (d, J = 7.2 Hz, 1H), 7.81 (s, 1H), 7.78-7.70 (m, 2H), 7.12 (d, J = 8.4 Hz, |
| | | 1H), 4.98 (s, 2H), 4.82 (d, J = 6.0 Hz, 2H), 4.28-4.18 (m, 2H), 4.14-4.00 (m, 2H), |
| | | 3.84-3.61 (m, 4H), 2.19-2.09 (m, 2H), 1.85-1.77 (m, 2H) ppm. |
| 186 | 562.1 | ¹H NMR (400 MHz, DMSO-d₆) δ = 9.64-9.61 (m, 1H), 9.40 (s, 1H), 8.70-8.64 (m, |
| | | 1H), 8.64-8.58 (m, 1H), 8.54 (d, J = 2.0 Hz, 1H), 8.27-8.25 (m, 1H), 7.88 (d, J = 7.2 |
| | | Hz, 1H), 7.81 (s, 1H), 7.77-7.70 (m, 2H), 7.07 (d, J = 8.4 Hz, 1H), 5.05-4.79 (m, 4H), |
| | | 4.28-4.19 (m, 2H), 3.97-3.84 (m, 2H), 3.73-3.56 (m, 4H), 2.05-1.93 (m, 2H), 1.86- |
| | | 1.71 (m, 2H) ppm. |
| 184 | 580.1 | ¹H NMR (400 MHz, DMSO-d₆) δ = 9.64 (s, 1H), 9.40 (s, 1H), 8.69-8.60 (m, 2H), 8.54 |
| | | (d, J = 2.0 Hz, 1H), 8.50-8.46 (m, 1H), 8.27-8.25 (m, 1H), 7.92 (d, J = 7.2 Hz, 1H), |
| | | 7.81 (s, 1H), 7.79-7.71 (m, 2H), 7.14 (d, J = 8.4 Hz, 1H), 5.01-4.96 (m, 2H), 4.82 (d, |
| | | J = 5.6 Hz, 2H), 4.24-4.21 (m, 2H), 3.87-3.81 (m, 4H), 3.71-3.64 (m, 2H), 2.12- |
| | | 2.02 (m, 4H) ppm. |
| 181 | 560.4 | ¹H NMR (400 MHz, DMSO-d₆) δ = 9.64-9.63 (m, 1H), 9.39 (s, 1H), 8.69-8.57 (m, |
| | | 2H), 8.54 (d, J = 1.6 Hz, 1H), 8.26-8.25 (m, 1H), 7.89 (d, J = 7.6 Hz, 1H), 7.81 (s, |
| | | 1H), 7.78-7.72 (m, 2H), 6.96 (d, J = 8.4 Hz, 1H), 4.98 (s, 2H), 4.82 (d, J = 5.6 Hz, |
| | | 2H), 4.53-4.43 (m, 1H), 4.27-4.19 (m, 2H), 4.09-4.08 (m, 1H), 4.01-3.97 (m, 1H), |
| | | 3.82-3.75 (m, 1H), 3.73-3.64 (m, 3H), 3.54-3.53 (m, 1H), 3.16-3.15 (m, 1H), 1.19 |
| | | (d, J = 6.8 Hz, 3H) ppm. |
| | | Chiral SFC: AD-3-MeOH+CAN (DEA)-50-3 mL-35T.lcm; Rt = 1.023 mins. |
| 178 | 560.1 | ¹H NMR (400 MHz, DMSO) δ = 9.70-9.58 (m, 1H), 9.42-9.37 (m, 1H), 8.70-8.58 |
| | | (m, 2H), 8.54 (d, J = 1.6 Hz, 1H), 8.318.24 (m, 1H), 7.89 (d, J = 7.2 Hz, 1H), 7.83- |
| | | 7.79 (m, 1H), 7.77-7.72 (m, 2H), 7.00-6.90 (m, 1H), 5.01-4.95 (m, 2H), 4.864.78 |
| | | (m, 2H), 4.52-4.42 (m, 1H), 4.27-4.20 (m, 2H), 4.13-3.95 (m, 2H), 3.81-3.75 (m, |
| | | 1H), 3.73-3.64 (m, 3H), 3.58-3.50 (m, 1H), 3.20-3.11 (m, 1H), 1.19 (d, J = 6.4 Hz, |
| | | 3H) ppm. |
| | | SFC: AD-3-EtOH (DEA)-60-3 mL-5 min-35T.lcm, Rt = 2.356 mins, ee % value = 100%. |
| 174 | 574.2 | ¹H NMR (400 MHz, DMSO-d₆) δ = 9.64-9.62 (m, 1H), 9.38 (s, 1H), 8.67-8.65 (m, 1H), |
| | | 8.61-8.59 (m, 1H), 8.53 (d, J = 1.6 Hz, 1H), 8.46-8.41 (m, 1H), 8.26 (s, 1H), 7.82- |
| | | 7.79 (m, 2H), 7.74-7.69 (m, 2H), 6.87 (d, J = 8.4 Hz, 1H), 4.98-4.97 (m, 2H), 4.81 (d, |
| | | J = 6.0 Hz, 2H), 4.73 (br t, J = 3.2 Hz, 1H), 4.23-4.20 (m, 2H), 3.98-3.98 (m, 2H), |
| | | 3.68-3.66 (m, 2H), 3.53 (br t, J = 11.2 Hz, 2H), 2.97 (s, 3H), 1.87-1.79 (m, 2H), |
| | | 1.63-1.60 (m, 2H) ppm. |
| 145 | 548.1 | ¹H NMR (400 MHz, DMSO-d₆) δ = 9.65-9.61 (m, 1H), 9.39 (d, J = 0.8 Hz, 1H), 8.68- |
| | | 8.66 (m, 1H), 8.61-8.59 (m, 1H), 8.54(d, J = 2.0 Hz, 1H), 8.40 (br s, 1H), 8.28 (s, 1H), |
| | | 7.83-7.81 (m, 2H), 7.75-7.69 (m, 2H), 6.82 (d, J = 8.4 Hz, 1H), 4.99 (s, 2H), 4.83 (d, |
| | | J = 5.6 Hz, 2H), 4.23 (br d, J = 4.8 Hz, 2H), 3.83 (t, J = 6.0 Hz, 2H), 3.70-3.67 (m, |
| | | 2H), 3.61 (t, J = 6.0 Hz, 2H), 3.28 (s, 3H), 3.15 (s, 3H) ppm. |
| 144 | 559.2 | ¹H NMR (400 MHz, DMSO-d₆) δ = 9.64-9.61 (m, 1H), 9.39 (d, J = 0.8 Hz, 1H), 8.67- |
| | | 8.60 (m, 2H), 8.53 (d, J = 2.0 Hz, 1H), 8.27-8.25 (m, 1H), 8.21 (s, 1H), 7.89-7.87 |
| | | (m, 1H), 7.80 (s, 1H), 7.89-7.74 (m, 2H), 7.01 (d, J = 8.4 Hz, 1H), 4.98 (s, 2H), 4.81 |
| | | (d, J = 5.6 Hz, 2H), 4.24-4.21 (m, 2H), 3.69-3.67 (m, 2H), 3.64-3.61 (m, 4H), 2.48- |
| | | 2.44 (m, 4H), 2.24 (s, 3H) ppm. |
| 228 | 546.3 | ¹H NMR (400 MHz, DMSO-d6) δ = 9.64-9.63 (m, 1H), 9.39 (s, 1H), 8.70-8.64 (m, |
| | | 1H), 8.63-8.58 (m, 1H), 8.54 (d, J = 1.6 Hz, 1H), 8.26 (d, J = 7.6 Hz, 1H), 7.89 (d, J = |
| | | 7.2 Hz, 1H), 7.81 (s, 1H), 7.76-7.68 (m, 2H), 6.58 (d, J = 8.0 Hz, 1H), 4.98 (s, 2H), |
| | | 4.82 (d, J = 5.6 Hz, 2H), 4.44-4.34 (m, 1H), 4.31-4.19 (m, 4H), 3.88 (d, J = 3.6 Hz, |
| | | 2H), 3.73-3.65 (m, 2H), 3.28 (s, 3H) ppm. |
| 226 | 546.3 | ¹H NMR (400 MHz, DMSO-d₆) δ = 9.64-9.62 (m, 1H), 9.39 (s, 1H), 8.67-8.61 (m, 2H), |
| | | 8.53 (s, 1H), 8.27-8.25 (m, 1H), 7.93 (d, J = 7.6 Hz, 1H), 7.81 (s, 1H), 7.78-7.73 (m, |
| | | 2H), 7.02 (d, J = 8.4 Hz, 1H), 4.98 (s, 2H), 4.82 (br d, J = 6.0 Hz, 2H), 4.24-4.21 (m, |
| | | 2H), 3.77-3.74 (m, 4H), 3.69-3.67 (m, 2H), 3.61-3.58 (m, 4H) ppm |

### Example 4. N-((2-(4-methyl-3,4-dihydro-2H-benzo[b][1,4]oxazin-8-yl)-1,6-naphthyridin-7-yl)methyl)-3,5-dihydro-2H-benzo[a][1,4]oxathiepine-8-carboxamide 1,1-dioxide

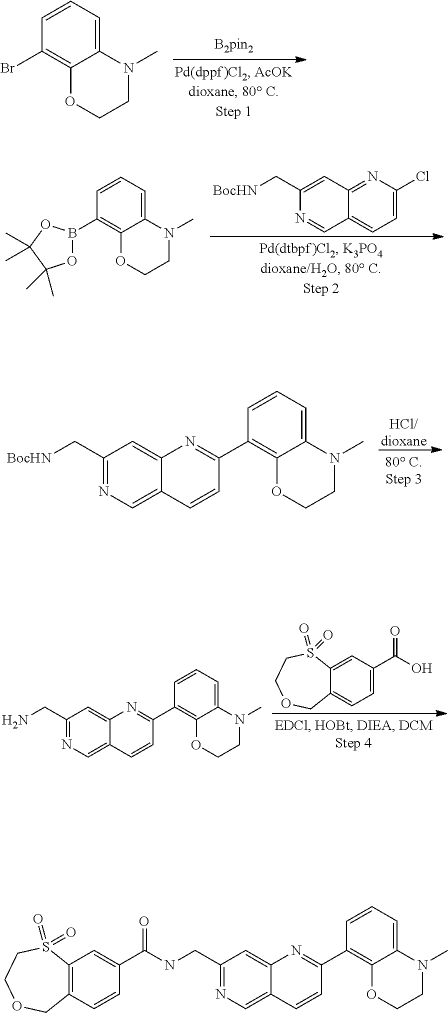

### Step 1: Preparation of 4-methyl-8-(4,4,5,5-tetramethyl-1,3,2-dioxaborolan-2-yl)-3,4-dihydro-2H-benzo[b][1,4]oxazine

Pd(dppf)Cl₂ (32.1 mg, 0.0448 mmol) and AcOK (129 mg, 1.32 mmol) were added to a solution of 4,4,5,5-tetramethyl-2-(4,4,5,5-tetramethyl-1,3,2-dioxaborolan-2-yl)-1,3,2-dioxaborolane (134 mg, 0.526 mmol) and 8-bromo-4-methyl-3,4-dihydro-2H-benzo[b][1,4]oxazine (100 mg, 0.438 mmol) in dioxane (2 mL). The reaction mixture was stirred at 80° C. for 2 h. The reaction mixture was diluted with H₂O (20 mL) and extracted with EA (20 mL×3). The combined organic layers were dried over anhydrous Na₂SO₄, filtered and concentrated under reduced pressure affording the title compound (125 mg, crude) as a brown oil. LCMS (ESI) m/z: [M+H]⁺=278.1

### Step 2: Preparation of tert-butyl ((2-(4-methyl-3,4-dihydro-2H-benzo[b][1,4]oxazin-8-yl)-1,6-naphthyridin-7-yl)methyl)carbamate

A mixture of tert-butyl-N-[(2-chloro-1,6-naphthyridin-7-yl)methyl]carbamate (100 mg, 0.340 mmol), 4-methyl-8-(4,4,5,5-tetramethyl-1,3,2-dioxaborolan-2-yl)-3,4-dihydro-2H-benzo[b][1,4]oxazine (122 mg, 0.443 mmol), K₃PO₄ (217 mg, 1.02 mmol), [1,1-Bis(di-tert-butylphosphino)ferrocene]dichloropalladium(II) (22.2 mg, 0.340 mmol) in dioxane (1 mL) and H₂O (0.3 mL) was degassed and purged with N₂ three times. The mixture was stirred at 80° C. for 2 h. The reaction mixture was diluted with H₂O (10 mL) and extracted with EA (10 mL×3). The combined organic layers were dried over anhydrous Na₂SO₄, filtered and concentrated under reduced pressure to get the residue. The residue was purified by reversed-phase HPLC (0.1% FA additive). The fractions were concentrated under reduced pressure to remove MeCN and then extracted with EA (50 mL×3). The combined organic layers were dried over anhydrous Na₂SO₄, filtered and concentrated under reduced pressure affording the title compound (100 mg, 0.205 mmol) as a yellow solid. LCMS (ESI) m/z: [M+H]⁺=407.3. ¹H NMR (400 MHz, DMSO-d6) δ=9.30 (s, 1H), 8.48 (d, J=8.8 Hz, 1H), 7.98 (d, J=8.4 Hz, 1H), 7.69 (s, 1H), 7.63-7.58 (m, 1H), 7.04-7.02 (m, 1H), 6.94-6.90 (m, 1H), 6.87-6.81 (m, 1H), 4.43 (d, J=5.8 Hz, 2H), 4.35-4.27 (m, 2H), 3.34-3.33 (m, 2H), 2.91 (s, 3H), 1.43 (s, 9H) ppm.

### Step 3: Preparation of (2-(4-methyl-3,4-dihydro-2H-benzo[b][1,4]oxazin-8-yl)-1,6-naphthyridin-7-yl)methanamine hydrochloride salt

HCl/dioxane (4N, 750 uL) was added to a solution of tert-butyl ((2-(4-methyl-3,4-dihydro-2H-benzo[b][1,4]oxazin-8-yl)-1,6-naphthyridin-7-ylmethylcarbamate (90 mg, 0.221 mmol) in dioxane (1 mL). The reaction mixture was stirred at 25° C. for 1 hr. The reaction mixture was concentrated under reduced pressure. The resulting residue was washed with MTBE (5 mL×2), filtered, and dried in vacuo affording the title compound (70 mg, 0.204 mmol) as a brown solid. LCMS (ESI) m/z: [M+H]+=307.2.

### Step 4: Preparation of N-((2-(4-methyl-3,4-dihydro-2H-benzo[b][1,4]oxazin-8-yl)-1,6-naphthyridin-7-yl)methyl)-3,5-dihydro-2H-benzo[a][1,4]oxathiepine-8-carboxamide 1,1-dioxide (58)

EDCl (25.2 mg, 0.131 mol), HOBt (17.7 mg, 0.131 mmol, DIEA (76.2 uL, 0.438 mmol) and (2-(4-methyl-3,4-dihydro-2H-benzo[b][1,4]oxazin-8-yl)-1,6-naphthyridin-7-yl)methanamine hydrochloride salt (30 mg, 0.0875 mmol) were added to a solution of 2,3-dihydro-5H-benzo[e][1,4]oxathiepine-8-carboxylic acid 1,1-dioxide (25.4 mg, 0.105 mmol) In DCM (0.5 mL) was added. The reaction mixture was stirred at 25° C. for 2 h. The reaction mixture was diluted with H₂O (5 mL) and extracted with DCM (5 mL×3). The combined organic layer was dried over anhydrous Na₂SO₄, filtered and concentrated under reduced pressure. The resulting residue was purified by reversed-phase HPLC (0.1% FA condition). The solution was concentrated under reduced pressure to remove MeCN and lyophilized affording the title compound (14.2 mg, 0.0257 mmol) as a yellow solid. LCMS (ESI) m/z: [M+H]+=531.2. ¹H NMR (400 MHz, CD₃OD) δ=9.30 (s, 1H), 8.64-8.59 (m, 1H), 8.48 (d, J=8.8 Hz, 1H), 8.22-8.20 (m, 1H), 8.00 (d, J=8.4 Hz, 1H), 7.93 (s, 1H), 7.83 (d, J=7.8 Hz, 1H), 7.01-8.92 (m, 2H), 8.88-8.83 (m, 1H), 5.04 (s, 2H), 4.92 (s, 2H), 4.35-4.31 (m, 4H), 3.53-3.50 (m, 2H), 3.34 (d, J=4.4 Hz, 2H), 2.97-2.92 (m, 3H) ppm.

The following examples in Table 4A were prepared using standard chemical manipulations and procedures similar to those used for the preparation of Example 4.

TABLE 4A
Compounds of the Invention.

| | LCMS | |
|---|---|---|
| # | (m/z) | 1H NMR |
| 227 | 460.0 | ¹HNMR (400 MHz, DMSO-d6) δ = 9.65-9.62 (m, 1H), 9.39 (s, 1H), 8.67 (d, J = 8.4 Hz, |
| | | 1H), 8.55 (d, J = 1.6 Hz, 1H), 8.34-8.24 (m, 4H), 7.82 (s, 1H), 7.75 (d, J = 7.6 Hz, 1H), |
| | | 7.61-7.52 (m, 3H), 4.99 (s, 2H), 4.83 (d, J = 5.6 Hz, 2H), 4.32-4.14 (m, 2H), 3.78- |
| | | 3.60 (m, 2H) ppm |
| 307 | 515.2 | ¹H NMR (400 MHz, CD₃OD) δ = 9.16 (s, 1H), 8.62 (d, J = 2.0 Hz, 1H), 8.46-8.33 (m, |
| | | 1H), 8.27-8.17 (m, 1H), 8.04 (d, J = 8.8 Hz, 1H), 7.97-7.92 (m, 2H), 7.85 (s, 1H), 7.69- |
| | | 7.61 (m, 2H), 6.64-6.53 (m, 1H), 5.05 (s, 2H), 4.89 (br s, 2H), 4.41-4.28 (m, 2H), |
| | | 3.57-3.51 (m, 2H), 3.48-3.43 (m, 2H), 3.09-2.99 (m, 2H), 2.86 (s, 3H) ppm |
| 196 | 531.3 | ¹H NMR (400 MHz, DMSO-d6) δ = 9.63-9.55 (m, 1H), 9.30 (s, 1H), 8.57-8.50 (m, |
| | | 2H), 8.28-8.19 (m, 2H), 7.78-7.71 (m, 2H), 7.62 (d, J = 2.0 Hz, 1H), 7.58-7.54 (m, |
| | | 1H), 6.82 (d, J = 8.4 Hz, 1H), 4.98 (s, 2H), 4.79 (d, J = 5.6 Hz, 2H), 4.34-4.27 (m, 2H), |
| | | 4.26-4.19 (m, 2H), 3.72-3.65 (m, 2H), 3.29 (d, J = 4.0 Hz, 2H), 2.96 (s, 3H) ppm |
| 193 | 515.2 | ¹H NMR (400 MHz, DMSO-d6) δ = 9.66-9.59 (m, 1H), 9.34 (s, 1H), 8.58 (d, J = 8.8 Hz, |
| | | 1H), 8.54 (d, J = 1.6 Hz, 1H), 8.28-8.20 (m, 2H), 7.79 (s, 1H), 7.74 (d, J = 8.0 Hz, 1H), |
| | | 7.55-7.53 (m, 1H), 7.38 (s, 1H), 7.20 (d, J = 7.6 Hz, 1H), 4.98 (s, 2H), 4.81 (d, J = 5.6 |
| | | Hz, 2H), 4.25-4.20 (m, 2H), 3.71-3.66 (m, 2H), 2.96-2.92 (m, 2H), 2.80 (s, 3H), 2.52- |
| | | 2.51 (m, 2H) ppm |
| 189 | 516.2 | ¹H NMR (400 MHz, DMSO-d6) δ = 9.80-9.78 (m, 1H), 9.37 (s, 1H), 9.28 (d, J = 2.0 Hz, |
| | | 1H), 8.80 (d, J = 2.0 Hz, 1H), 8.61 (d, J = 8.8 Hz, 1H), 8.22 (d, J = 8.4 Hz, 1H), 7.86 (s, |
| | | 1H), 7.55 (d, J = 7.6 Hz, 1H), 7.36-7.32 (m, 1H), 7.27-7.26 (m, 1H), 6.59-6.57 (m, |
| | | 1H), 5.06 (s, 2H), 4.83 (d, J = 6.0 Hz, 2H), 4.31-4.27 (m, 2H), 3.89 (d, J = 14, 4 Hz, |
| | | 4H), 3.85 (s, 2H), 2.35-2.32 (m, 2H) ppm |
| 249 | 501.2 | ¹H NMR (400 MHz, DMSO-d6) δ = 9.82-9.79 (m, 1H), 9.38 (s, 1H), 9.29 (d, J = 2.0 Hz, |
| | | 1H), 8.81 (d, J = 2.0 Hz, 1H), 8.64 (d, J = 8.4 Hz, 1H), 8.28 (d, J = 8.4 Hz, 1H), 8.04 (d, |
| | | J = 8.0 Hz, 1H), 7.99 (s, 1H), 7.87 (s, 1H), 7.45-7.41 (m, 1H), 7.23 (d, J = 7.6 Hz, 1H), |
| | | 5.07 (s, 2H), 4.84 (d, J = 5.8 Hz, 2H), 4.32-4.26 (m, 2H), 3.87-3.80 (m, 2H), 2.10- |
| | | 2.02 (m, 1H), 1.04-0.97 (m, 2H), 0.81-0.75 (m, 2H)ppm |
| 246 | 513.3 | ¹H NMR (400 MHz, DMSO-d6) δ = 9.65-9.62 (m, 1H), 9.36 (s, 1H), 8.72-8.68 (m, |
| | | 1H), 8.63 (d, J = 8.4 Hz, 1H), 8.50 (d, J = 1.2 Hz, 1H), 8.26 (d, J = 8.8 Hz, 1H), 8.18- |
| | | 8.17 (m, 1H), 8.04-8.01 (m, 1H), 7.97 (s, 1H), 7.81 (s, 1H), 7.62 (d, J = 8.4 Hz, 1H), |
| | | 7.43 (t, J = 7.6 Hz, 1H), 7.23 (d, J = 7.6 Hz, 1H), 4.90 (s, 2H), 4.80 (d, J = 5.6 Hz, 2H), |
| | | 4.72 (d, J = 5.6 Hz, 2H), 2.07-2.05 (m, 1H), 1.02-0.99 (m, 2H), 0.78-0.76 (m, 2H) ppm. |
| 262 | 522.2 | ¹H NMR (400 MHz, DMSO-d₆) δ = 9.70 (s, 1H), 9.67-9.64 (m, 1H), 9.48 (s, 1H), 8.99 |
| | | (s, 1H), 8.80-8.76 (m, 1H), 8.65-8.63(m, 1H), 8.53 (d, J = 1.6 Hz, 1H), 8.47(s, 1H), 8.27- |
| | | 8.25 (m, 1H), 7.91 (s, 1H), 7.74 (d, J = 7.6 Hz, 1H), 4.98 (s, 2H), 4.84 (d, J = 5.6 Hz, |
| | | 2H), 4.24-4.21 (m, 2H), 3.69-3.66 (m, 2H), 1.89-1.77 (m, 6H) ppm. |
| 209 | 530.2 | ¹H NMR (400 MHz, DMSO-d₆) δ = 10.03 (s, 1H), 9.69-9.65 (m, 1H), 9.52 (s, 1H), 9.37 |
| | | (s, 1H), 8.84 (d, J = 8.4 Hz, 1H), 8.61-8.52 (m, 2H), 8.28-8.25 (m, 1H), 7.94 (s, 1H), |
| | | 7.75 (d, J = 8.0 Hz, 1H), 4.98 (s, 2H), 4.86 (d, J = 5.6 Hz, 2H), 4.27-4.20 (m, 2H), 3.71- |
| | | 3.66 (m, 2H) ppm. |
| 305 | 502.4 | ¹H NMR (400 MHz, DMSO-d6) δ = 9.65-9.62 (m, 1H), 9.45 (s, 2H), 8.78 (s, 1H), 8.74 |
| | | (d, J = 8.8 Hz, 1H), 8.56-8.53 (m, 2H), 8.27-8.25 (m, 1H), 7.88 (s, 1H), 7.74 (d, J = |
| | | 7.6 Hz, 1H), 4.98 (s, 2H), 4.83 (d, J = 5.6 Hz, 2H), 4.24-4.21 (m, 2H), 3.69-3.66 (m, |
| | | 2H), 3.30 (s, 3H), 2.36-2.34 (m, 1H), 1.16-1.14 (m, 4H) ppm. |
| 182 | 567.2 | ¹H NMR (400 MHz, DMSO-d6) δ = 9.69-9.68 (m, 1H), 9.43 (s, 1H), 8.94 (s, 1H), 8.72 |
| | | (d, J = 8.4 Hz, 1H), 8.61 (d, J = 2.4 Hz, 1H), 8.58 (d, J = 8.8 Hz, 1H), 8.49 (s, 1H), 8.32- |
| | | 8.30 (m, 1H), 7.94 (d, J = 8.4 Hz, 1H), 7.86 (s, 1H), 4.83 (d, J = 5.6 Hz, 2H), 4.40 (br |
| | | d, J = 11.2 Hz, 2H), 3.73-3.66 (m, 2H), 3.43 (s, 3H), 2.62-2.56 (m, 2H), 1.21 (d, J = |
| | | 6.0 Hz, 6H) ppm. |
| 163 | 583.2 | ¹H NMR (400 MHz, DMSO-d6) δ = 9.73-9.71 (m, 1H), 9.44 (d, J = 0.4 Hz, 1H), 8.95 (s, |
| | | 1H), 8.73-8.70 (m, 1H), 8.60-8.56 (m, 2H), 8.49-8.44 (m, 2H), 8.10 (d, J = 8.0 Hz, |
| | | 1H), 7.89 (s, 1H), 7.83-7.56 (m, 1H), 4.85 (d, J = 5.6 Hz, 2H), 4.41 (br d, J = 11.2 Hz, |
| | | 2H), 3.71-3.66 (m, 2H), 3.38 (s, 3H), 2.62-2.56 (m, 2H), 1.22 (d, J = 6.4 Hz, 6H) ppm. |
| 230 | 501.2 | ¹H NMR (400 MHz, DMSO-d6) δ = 9.78-9.74 (m, 1H), 9.45 (d, J = 2.0 Hz, 2H), 9.25 |
| | | (d, J = 2.0 Hz, 1H), 8.79 (s, 1H), 8.77-8.72 (m, 2H), 8.55 (d, J = 8.4 Hz, 1H), 7.92 (s, |
| | | 1H), 4.85 (d, J = 5.6 Hz, 2H), 3.56-3.51 (m, 2H), 3.40-3.35 (m, 2H), 2.39-2.33 (m, |
| | | 1H), 2.22-2.12 (m, 2H), 1.91-1.75 (m, 2H), 1.18-1.13 (m, 4H) ppm. |
| 236 | 502.3 | ¹H NMR (400 MHz, DMSO-d6) δ = 9.63-9.61 (m, 1H), 9.51-9.41 (m, 2H), 8.79 (s, |
| | | 1H), 8.75 (d, J = 8.4 Hz, 1H), 8.56 (d, J = 8.4 Hz, 1H), 8.51-8.48 (m, 1H), 8.24-8.22 |
| | | (m, 7.8 Hz, 1H), 7.87 (s, 1H), 7.66 (d, J = 7.9 Hz, 1H), 5.00 (s, 2H), 4.84 (d, J = 5.6 Hz, |
| | | 2H), 4.09-3.97 (m, 2H), 3.48-3.41 (m, 2H), 2.34 (s, 1H), 1.22-1.10 (m, 4H) ppm |
| 269 | 508.3 | ¹H NMR (400 MHz, DMSO-d6) δ = 9.65-9.62 (m, 1H), 9.44 (s, 2H), 8.78 (s, 1H), 8.75 |
| | | (d, J = 8.8 Hz, 1H), 8.55 (d, J = 8.8 Hz, 1H), 8.50 (d, J = 2.0 Hz, 1H), 8.42 (d, J = 1.6 Hz, |
| | | 1H), 7.89 (s, 1H), 4.83 (d, J = 6.0 Hz, 2H), 3.34 (s, 3H), 2.75 (s, 3H), 2.35-2.32 (m, |
| | | 1H), 1.15-1.14 (m, 4H) ppm. |
| 272 | 512.0 | ¹H NMR (400 MHz, DMSO-d₆) δ = 9.73 (br s, 1H), 9.49-9.39 (m, 2H), 8.83-8.69 (m, |
| | | 2H), 8.55 (d, J = 8.8 Hz, 1H), 8.47 (s, 1H), 8.36 (d, J = 9.2 Hz, 1H), 7.97-7.86 (m, 1H), |
| | | 4.83 (d, J = 5.6 Hz, 2H), 3.46 (s, 3H), 2.37-2.31 (m, 1H), 1.15 (d, J = 6.0 Hz, 4H) ppm. |
| 298 | 475.3 | ¹H NMR (400 MHz, DMSO-d₆) δ = 9.74-9.71 (m, 1H), 9.45 (d, J = 2.0 Hz, 2H), 9.28 (d, |
| | | J = 2.0 Hz, 1H), 8.79 (s, 1H), 8.75-8.73 (m, 2H), 8.55 (d, J = 8.4 Hz, 1H), 7.93 (s, 1H), |
| | | 4.85 (d, J = 5.6 Hz, 2H), 3.38 (s, 3H), 2.91 (s, 3H), 2.39-2.34 (m, 1H), 1.18-1.13 (m,4H) ppm. |
| 302 | 502.1 | ¹H NMR (400 MHz, DMSO-d6) δ = 9.63-9.52 (m, 1H), 9.48-9.38 (m, 2H), 8.80-8.78 |
| | | (m, 1H), 8.77-8.73 (m, 1H), 8.58-8.53 (m, 1H), 8.49-8.46 (m, 1H), 8.31-8.22 (m, |
| | | 1H), 7.90-7.78 (m, 1H), 7.49-7.31 (m, 1H), 4.86-4.78 (m, 2H), 4.32-4.24(m, 2H), |
| | | 3.66-3.59 (m, 2H), 2.38-2.32 (m, 1H), 2.32-2.24 (m, 2H), 1.18-1.13 (m, 4H) ppm. |
| 288 | 523.3 | ¹H NMR (400 MHz, DMSO-d6) δ = 9.67-9.64 (m, 1H), 9.44 (s, 2H), 8.78 (s, 1H), 8.75 |
| | | (d, J = 8.8 Hz, 1H), 8.55 (d, J = 8.4 Hz, 1H), 8.51 (d, J = 2.0 Hz, 1H), 8.25-8.22 (m, |
| | | 1H), 7.90-7.87 (m, 2H), 4.83 (d, J = 5.6 Hz, 2H), 2.84 (s, 6H), 2.34 (d, J = 6.8 Hz, 1H), |
| | | 1.16-1.14 (m, 4H) ppm. |
| 188 | 513.3 | ¹H NMR (400 MHz, DMSO-d6) δ = 9.65-9.62 (m, 1H), 9.36 (s, 1H), 8.72-8.68 (m, |
| | | 1H), 8.63 (d, J = 8.4 Hz, 1H), 8.50 (d, J = 1.2 Hz, 1H), 8.26 (d, J = 8.8 Hz, 1H), 8.18- |
| | | 8.17 (m, 1H), 8.04-8.01 (m, 1H), 7.97 (s, 1H), 7.81 (s, 1H), 7.62 (d, J = 8.4 Hz, 1H), |
| | | 7.43 (t, J = 7.6 Hz, 1H), 7.23 (d, J = 7.6 Hz, 1H), 4.90 (s, 2H), 4.80 (d, J = 5.6 Hz, 2H), |
| | | 4.72 (d, J = 5.6 Hz, 2H), 2.07-2.05 (m, 1H), 1.02-0.99 (m, 2H), 0.78-0.76 (m, 2H) ppm. |
| 197 | 530.2 | ¹H NMR (400 MHz, DMSO-d6) δ = 9.64-9.61 (m, 1H), 9.39 (s, 1H), 8.69-8.64 (m, |
| | | 1H), 8.62-8.58 (m, 1H), 8.52 (d, J = 1.6 Hz, 1H), 8.27-8.26 (m, 1H), 7.87 (d, J = 7.2 |
| | | Hz, 1H), 7.82 (s, 1H), 7.77-7.67 (m, 2H), 6.53 (d, J = 8.0 Hz, 1H), 4.96 (s, 2H), 4.81 (d, |
| | | J = 6.0 Hz, 2H), 4.31-4.27 (m, 1H), 4.12-3.98 (m, 5H), 3.71-3.59 (m, 1H), 2.41- |
| | | 2.35 (m, 2H), 1.16 (d, J = 7.2 Hz, 3H) ppm. |
| | | Chiral SFC: AD-3-IPA + CAN (DEA)-40-5 min-3 mL-35T.lcm; Rt = 2.838 mins, ee % = 100.00%. |
| 199 | 530.2 | ¹H NMR (400 MHz, DMSO-d6) δ = 9.64-9.62 (m, 1H), 9.39 (s, 1H), 8.68-8.64 (m, |
| | | 1H), 8.62-8.58 (m, 1H), 8.52 (d, J = 2.0 Hz, 1H), 8.28-8.26 (m, 1H), 7.87 (d, J = 7.2 |
| | | Hz, 1H), 7.82 (s, 1H), 7.77-7.67 (m, 2H), 6.53 (d, J = 7.6 Hz, 1H), 4.96 (s, 2H), 4.82 (d, |
| | | J = 6.0 Hz, 2H), 4.31-4.27 (m, 1H), 4.12-3.97 (m, 5H), 3.70-3.61 (m, 1H), 2.41- |
| | | 2.36 (m, 2H), 1.16 (d, J = 6.8 Hz, 3H) ppm. |
| | | Chiral SFC: AD-3-IPA + CAN (DEA)-40-5 min-3 mL-35T.lcm; Rt = 3.552 mins. |
| 287 | 520.2 | ¹H NMR (400 MHz, DMSO-d6) δ = 9.73-9.70 (m, 1H), 9.45 (s, 2H), 8.78-8.73 (m, |
| | | 2H), 8.55 (d, J = 8.4 Hz, 1H), 8.41-8.40 (m, 1H), 8.19-8.16 (m, 1H), 7.90 (s, 1H), 5.02 |
| | | (s, 2H), 4.83 (d, J = 5.6 Hz, 2H), 4.24-4.21 (m, 2H), 3.77-3.75 (m, 2H), 2.38-2.33 (m, |
| | | 1H), 1.16-1.14 (m, 4H) ppm. |
| 187 | 536.1 | ¹H NMR (400 MHz, DMSO-d6) δ = 9.74-9.71 (m, 1H), 9.45(s, 2H), 8.78-8.73 (m, |
| | | 2H), 8.57-8.50 (m, 2H), 8.42-8.40 (m, 2H), 7.91 (s, 1H), 5.20 (s, 2H), 4.83 (d, J = 5.6 |
| | | Hz, 2H), 4.24-4.22 (m, 2H), 3.79-3.77 (m, 2H), 2.38-2.33 (m, 1H), 1.16-1.14 (m,4H) ppm |
| 293 | 501.4 | ¹H NMR (400 MHz, DMSO-d6) δ = 9.58-9.52 (m, 1H), 9.45 (d, J = 1.2 Hz, 2H), 8.79 |
| | | (s, 1H), 8.77-8.71 (m, 1H), 8.55 (d, J = 8.8 Hz, 1H), 8.49-8.37 (m, 1H), 8.19-8.05 |
| | | (m, 1H), 7.84 (s, 1H), 7.57 (d, J = 8.0 Hz, 2H), 4.82 (d, J = 6.0 Hz, 2H), 3.41 (br s, 2H), |
| | | 3.29-3.28 (m, 1H), 3.28-3.23 (m, 2H), 2.41-2.29 (m, 1H), 1.73 (br s, 2H), 1.20-1.09 |
| | | (m, 4H) ppm. |
| 297 | 515.4 | ¹H NMR (400 MHz, DMSO-d6) δ = 9.60-9.50 (m, 1H), 9.44 (s, 2H), 8.81-8.69 (m, |
| | | 2H), 8.53 (d, J = 8.4 Hz, 1H), 8.36 (d, J = 1.6 Hz, 1H), 8.22-8.08 (m, 1H), 7.87 (s, 1H), |
| | | 7.61 (d, J = 8.0 Hz, 1H), 4.81 (br d, J = 5.6 Hz, 2H), 3.82-3.50 (m, 2H), 3.24 (br s, 2H), |
| | | 2.57 (s, 3H), 2.40-2.25 (m, 1H), 1.79 (br d, J = 5.2 Hz, 2H), 1.22-1.09 (m, 4H) ppm. |
| 252 | 536.3 | ¹H NMR (400 MHz, DMSO-d6) δ = 9.64 (s, 1H), 9.39 (s, 1H), 8.67 (d, J = 8.4 Hz, 1H), |
| | | 8.53 (d, J = 1.6 Hz, 1H), 8.34-8.17 (m, 4H), 7.82 (s, 1H), 7.74 (d, J = 8.0 Hz, 1H), 7.53 |
| | | (d, J = 8.0 Hz, 1H), 7.45 (s, 1H), 4.98 (s, 2H), 4.82 (d, J = 5.6 Hz, 2H), 4.25-4.20 (m, |
| | | 2H), 3.69-3.66 (m, 2H), 3.11 (s, 1H), 2.14-1.98 (m, 2H) ppm. |
| | | Chiral SFC: IG-3-MeOH+ACN(DEA)-60-3ML-7MIN-35T.lcm, Rt = 3.596 min, ee % = 100%. |
| 251 | 536.3 | ¹H NMR (400 MHz, DMSO-d6) δ = 9.60 (s, 1H), 9.39 (s, 1H), 8.66 (s, 1H), 8.53 (d, J = |
| | | 2.0 Hz, 1H), 8.34-8.16 (m, 4H), 7.82 (s, 1H), 7.74 (d, J = 8.0 Hz, 1H), 7.54 (s, 1H), |
| | | 7.48-7.40 (m, 1H), 4.98 (s, 2H), 4.82 (d, J = 5.6 Hz, 2H), 4.22 (d, J = 5.6 Hz, 2H), 3.69- |
| | | 3.66 (m, 2H), 3.11 (s, 1H), 2.17-1.96 (m, 2H) ppm. |
| | | Chiral SFC: IG-3-MeOH+ACN(DEA)-60-3ML-7MIN-35T.lcm, Rt = 4.550 min, ee % = 96.31%. |
| 286 | 501.3 | ¹H NMR (400 MHz, DMSO-d6) δ = 9.59-9.56 (m, 1H), 9.45-9.45 (m, 2H), 8.79 (s, |
| | | 1H), 8.76-8.74 (m, 1H), 8.58 (d, J = 1.8 Hz, 1H), 8.56 (d, J = 8.8 Hz, 1H), 8.18-8.16 |
| | | (m, 1H), 7.86 (s, 1H), 7.65 (d, J = 8.0 Hz, 1H), 5.11 (d, J = 13.2 Hz, 1H), 4.92 (d, J = |
| | | 13.2 Hz, 1H), 4.84 (d, J = 5.6 Hz, 2H), 4.76 (s, 1H), 4.26-4.19 (m, 2H), 3.44-3.41 (m, |
| | | 2H), 2.39-2.35 (m, 1H), 1.16-1.14 (m, 4H) ppm. |

The following examples in Table 4B were prepared using standard chemical manipulations and procedures similar to those used above.

TABLE 4B
Compounds of the Invention.

| | LCMS | |
|---|---|---|
| # | (ESI/M + H) | 1H NMR |
| 309 | 516.1 | 1H NMR (400 MHz, DMSO-d6) δ 9.75 (t, J = 5.8 Hz, 1H), 8.90 (dd, J = 9.1, 1.0 Hz, |
| | | 1H), 8.52 (dd, J = 5.5, 3.6 Hz, 2H), 8.25 (dd, J = 7.8, 1.9 Hz, 1H), 8.17 (d, J = 1.0 |
| | | Hz, 1H), 7.73 (d, J = 7.9 Hz, 1H), 7.66-7.58 (m, 1H), 7.44-7.26 (m, 2H), 6.68-6.59 |
| | | (m, 1H), 5.12 (d, J = 5.7 Hz, 2H), 4.97 (s, 2H), 4.28-4.21 (m, 2H), 3.91 (t, J = 7.2 |
| | | Hz, 4H), 3.72-3.65 (m, 2H), 2.40-2.29 (m, 2H), |
| 310 | 574.3 | 1H NMR (400 MHz, Methanol-d4) δ 8.79 (d, J = 9.0 Hz, 1H), 8.58 (d, J = 1.9 Hz, |
| | | 1H), 8.41 (d, J = 9.1 Hz, 1H), 8.27-8.14 (m, 2H), 7.86-7.81 (m, 1H), 7.69-7.58 (m, |
| | | 2H), 7.42 (t, J = 8.0 Hz, 1H), 7.16 (dd, J = 8.3, 2.5 Hz, 1H), 5.19 (s, 2H), 5.02 (s, |
| | | 2H), 4.37-4.28 (m, 2H), 3.86-3.75 (m, 2H), 3.65 (dd, 2H), 3.58-3.48 (m, 2H), 2.39 |
| | | (dd, J = 11.9, 10.3 Hz, 2H), 1.23 (d, J = 6.3 Hz, 6H), |
| 311 | 574.3 | 1H NMR (400 MHz, METHANOL-d4) δ = 9.31 (s, 1H), 8.71 (d, J = 8.8 Hz, 1H), 8.64 |
| | | (d, J = 1.8 Hz, 1H), 8.58 (d, J = 8.6 Hz, 1H), 8.48 (s, 1H), 8.24-8.22 (m, 1H), 7.98 |
| | | (s, 1H), 7.91 (d, J = 7.4 Hz, 1H), 7.77-7.63 (m, 2H), 7.33 (d, J = 8.4 Hz, 1H), 5.07 |
| | | (s, 2H), 4.95 (s, 2H), 4.40-4.33 (m, 2H), 3.62-3.53 (m, 3H), 3.44 (s, 3H), 3.36 (s, |
| | | 1H), 3.28 (s, 3H), 2.53-2.51 (m, 1H), 1.43-1.41 (m, 1H), 1.02-0.89 (m, 2H) ppm |
| 312 | 574.3 | 1H NMR (400 MHz, METHANOL-d4) δ = 9.29 (s, 1H), 8.69 (d, J = 8.8 Hz, 1H), 8.62 |
| | | (d, J = 1.8 Hz, 1H), 8.56 (d, J = 8.4 Hz, 1H), 8.47 (s, 1H), 8.22 8.20 (m, 1H), 7.96 |
| | | (s, 1H), 7.89 (d, J = 7.4 Hz, 1H), 7.73-7.61 (m, 2H), 7.31 (d, J = 8.4 Hz, 1H), 5.05 |
| | | (s, 2H), 4.93 (s, 2H), 4.41-4.30 (m, 2H), 3.60-3.50 (m, 3H), 3.42 (s, 3H), 3.34 (br |
| | | s, 1H), 3.26 (s, 3H), 2.51-2.50 (m, 1H), 1.46-1.37 (m, 1H), 0.99-0.89 (m, 2H) ppm |
| 313 | 589.3 | 1H NMR (400 MHz, DMSO-d6) δ = 9.62-9.60 (m, 1H), 9.36 (s, 1H), 8.54-8.49 (m, |
| | | 2H), 8.43 (s, 1H), 8.24-8.22 (m, 1H), 7.88-7.70 (m, 3H), 7.09-6.96 (m, 3H), 4.97 |
| | | (s, 2H), 4.80 (br d, J = 5.6 Hz, 2H), 4.26-4.17 (m, 2H), 4.11-4.09 (m, 2H), 3.70- |
| | | 3.64 (m, 2H), 3.57-3.52 (m, 2H), 3.41-3.40 (m, 5H), 3.29 (s, 3H), 2.01-1.91 (m, 2H) ppm |
| 314 | 578.4 | 1H NMR (400 MHz, METHANOL-d4) δ = 9.30 (s, 1H), 8.71 (d, J = 8.8 Hz, 1H), 8.62 |
| | | (d, J = 1.6 Hz, 1H), 8.58 (d, J = 8.8 Hz, 1H), 8.22-8.20 (m, 1H), 8.00-7.89 (m, |
| | | 2H), 7.79-7.69 (m, 1H), 7.64 (d, J = 8.0 Hz, 1H), 6.85 (d, J = 8.4 Hz, 1H), 5.05 (s, |
| | | 2H), 4.93 (s, 2H), 4.82-4.69 (m, 4H), 4.46-4.31 (m, 4H), 3.60-3.49 (m, 2H), 3.18 |
| | | (s, 3H) ppm |
| 315 | 530.2 | 1H NMR (400 MHz, DMSO-d6) δ = 9.60-9.57 (m, 1H), 9.05 (s, 1H), 8.54 (d, J = 1.6 |
| | | Hz, 1H), 8.27-8.24 (m, 1H), 8.22 (s, 1H), 8.16 (d, J = 9.2 Hz, 1H), 8.01 (d, J = 9.2 |
| | | Hz, 1H), 7.74 (d, J = 7.6 Hz, 1H), 7.48-7.47 (m, 2H), 6.91 (d, J = 7.6 Hz, 1H), 4.98 |
| | | (s, 2H), 4.74 (br d, J = 6.0 Hz, 2H), 4.24-4.21 (m, 2H), 4.13-4.10 (m, 2H), 3.70-3.67 |
| | | (m, 2H), 2.78-2.76 (m, 2H), 2.35 (s, 3H), 1.96-1.90 (m, 2H) ppm |
| 316 | 554.2 | 1H NMR (400 MHz, DMSO-d6) δ = 9.68-9.57 (m, 1H), 9.37 (s, 1H), 8.58-8.50 (m, |
| | | 2H), 8.28-8.23 (m, 1H), 8.06 (d, J = 8.8 Hz, 1H), 7.83-7.70 (m, 3H), 7.19 (s, 1H), |
| | | 7.05 (d, J = 8.0 Hz, 1H), 6.53-6.15 (m, 1H), 4.97 (s, 2H), 4.81 (d, J = 5.6 Hz, 2H), |
| | | 4.29-4.18 (m, 2H), 3.87 (s, 3H), 3.73-3.63 (m, 2H), 3.29-3.22 (m, 2H) ppm |
| 317 | 576.3 | 1H NMR (400 MHz, DMSO-d6) δ = 9.65-9.63 (m, 1H), 9.39 (s, 1H), 8.71-8.57 (m, |
| | | 2H), 8.54 (d, J = 1.6 Hz, 1H), 8.27-8.25 (m, 1H), 7.85-7.78 (m, 2H), 7.77-7.64 |
| | | (m, 2H), 7.04 (d, J = 8.4 Hz, 1H), 4.98 (s, 2H), 4.82 (d, J = 5.6 Hz, 2H), 4.31-4.26 |
| | | (m, 2H), 4.25-4.20 (m, 2H), 3.71-3.66 (m, 2H), 3.24-3.05 (m, 2H), 1.95-1.61 |
| | | (m, 4H), 1.45-1.36 (m, 3H) ppm |
| 318 | 576.3 | 1H NMR (400 MHz, DMSO-d6) δ = 9.65-9.63 (m, 1H), 9.39 (s, 1H), 8.71-8.57 (m, |
| | | 2H), 8.54 (d, J = 1.6 Hz, 1H), 8.27-8.25 (m, 1H), 7.85-7.78 (m, 2H), 7.77-7.64 |
| | | (m, 2H), 7.04 (d, J = 8.4 Hz, 1H), 4.98 (s, 2H), 4.82 (d, J = 5.6 Hz, 2H), 4.31-4.26 |
| | | (m, 2H), 4.25-4.20 (m, 2H), 3.71-3.66 (m, 2H), 3.24-3.05 (m, 2H), 1.95-1.61 |
| | | (m, 4H), 1.45-1.36 (m, 3H) ppm |
| 319 | 588.5 | 1H NMR (400 MHz, METHANOL-d4) δ = 9.22-9.16 (m, 1H), 8.56-8.43 (m, 3H), |
| | | 8.15-8.05 (m, 1H), 7.85-7.82 (m, 1H), 7.81-7.77 (m, 1H), 7.65-7.59 (m, 1H), |
| | | 7.50-7.46 (m, 1H), 6.87-6.82 (m, 1H), 4.82 (br s, 2H), 4.73-4.66 (m, 1H), 4.30- |
| | | 4.16 (m, 3H), 3.77-3.59 (m, 4H), 3.17-3.06 (m, 1H), 2.52-2.39 (m, 2H), 1.53- |
| | | 1.48 (m, 3H), 1.21-1.18 (m, 6H) ppm |
| 320 | 554.2 | 1H NMR (400 MHz, DMSO-d6) δ = 9.70-9.57 (m, 1H), 9.40 (s, 1H), 8.59 (d, J = |
| | | 8.4 Hz, 1H), 8.51 (d, J = 2.0 Hz, 1H), 8.43 (s, 1H), 8.34-8.21 (m, 1H), 8.07 (d, J = |
| | | 8.8 Hz, 1H), 7.90 (d, J = 8.0 Hz, 1H), 7.81 (s, 1H), 7.74 (d, J = 8.0 Hz, 1H), 7.40 (s, |
| | | 1H), 7.31 (d, J = 7.6 Hz, 1H), 7.26-6.95 (m, 1H), 4.95 (s, 2H), 4.81 (brd, J = 6.0 |
| | | Hz, 2H), 4.37-4.22 (m, 1H), 4.08-3.96 (m, 1H), 3.91 (s, 3H), 3.72-3.58 (m, 1H), |
| | | 1.15 (d, J = 6.8 Hz, 3H) ppm |
| 321 | 556.2 | 1H NMR (400 MHz, DMSO-d6) δ = 9.70-9.60 (m, 1H), 9.39 (s, 1H), 8.69-8.58 (m, |
| | | 2H), 8.54 (d, J = 1.6 Hz, 1H), 8.42 (s, 1H), 8.31-8.23 (m, 1H), 7.83-7.78 (m, 2H), |
| | | 7.74 (d, J = 7.6 Hz, 1H), 7.70-7.64 (m, 1H), 6.84 (d, J = 8.4 Hz, 1H), 4.98 (s, 2H), |
| | | 4.82 (br d, J = 5.6 Hz, 2H), 4.31-4.16 (m, 2H), 4.05-3.96 (m, 1H), 3.933.85 (m, |
| | | 1H), 3.74-3.62 (m, 2H), 3.55-3.45 (m, 2H), 2.17-1.99 (m, 1H), 1.91-1.76 (m, |
| | | 1H), 1.21-1.05 (m, 2H), 0.70-0.55 (m, 1H), 0.35-0.17 (m, 1H) ppm |
| 322 | 556.2 | 1H NMR (400 MHz, DMSO-d6) δ = 9.70-9.60 (m, 1H), 9.40 (s, 1H), 8.69-8.58 (m, |
| | | 2H), 8.54 (d, J = 1.6 Hz, 1H), 8.42 (s, 1H), 8.31-8.22 (m, 1H), 7.83-7.78 (m, 2H), |
| | | 7.74 (d, J = 7.6 Hz, 1H), 7.70-7.64 (m, 1H), 6.84 (d, J = 8.4 Hz, 1H), 4.98 (s, 2H), |
| | | 4.82 (br d, J = 5.6 Hz, 2H), 4.31-4.15 (m, 2H), 4.05-3.95 (m, 1H), 3.93-3.85 (m, |
| | | 1H), 3.74-3.62 (m, 2H), 3.55-3.45 (m, 2H), 2.17-1.99 (m, 1H), 1.91-1.77 (m, |
| | | 1H), 1.21-1.05 (m, 2H), 0.70-0.55 (m, 1H), 0.35-0.17 (m, 1H) ppm |
| 323 | 574.3 | 1H NMR (400 MHz, DMSO-d6) δ = 9.64-9.61 (m, 1H), 9.38 (s, 1H), 8.71-8.64 (m, |
| | | 1H), 8.62-8.57 (m, 1H), 8.54 (d, J = 1.6 Hz, 1H), 8.27-8.25 (m, 1H), 7.86-7.78 |
| | | (m, 2H), 7.77-7.65 (m, 2H), 6.84 (d, J = 8.4 Hz, 1H), 4.98 (s, 2H), 4.82 (d, J = 6.0 |
| | | Hz, 2H), 4.29-4.18 (m, 2H), 3.99 (s, 2H), 3.71-3.65 (m, 2H), 3.20 (s, 3H), 3.15 (s, |
| | | 3H), 0.77-0.66 (m, 4H) ppm |
| 324 | 572.3 | 1H NMR (400 MHz, DMSO-d6) δ = 9.58-9.56 (m, 1H), 9.40 (s, 1H), 8.72-8.56 (m, |
| | | 2H), 8.50-8.39 (m, 1H), 8.21-8.11 (m, 1H), 7.92 (d, J = 7.6 Hz, 1H), 7.83-7.70 |
| | | (m, 2H), 7.58-7.48 (m, 1H), 7.08-6.99 (m, 1H), 4.87-4.73 (m, 2H), 4.40-4.23 |
| | | (m, 2H), 3.72-3.63 (m, 3H), 3.12 (br d, J = 4.2 Hz, 2H), 2.66 (br s, 2H), 2.32-2.10 |
| | | (m, 2H), 1.37 (br d, J = 6.8 Hz, 3H), 1.22 (br d, J = 6.2 Hz, 6H) ppm |
| 325 | 602.3 | 1H NMR (400 MHz, DMSO-d6) δ = 9.45-9.33 (m, 2H), 8.71-8.58 (m, 2H), 8.51 |
| | | (d, J = 1.6 Hz, 1H), 8.46 (br s, 1H), 8.28-8.25 (m, 1H), 7.97-7.88 (m, 2H), 7.81- |
| | | 7.68 (m, 2H), 7.04 (d, J = 8.8 Hz, 1H), 5.52-5.38 (m, 1H), 4.96 (s, 2H), 4.41-4.23 |
| | | (m, 3H), 4.02-4.00 (m, 1H), 3.74-3.59 (m, 3H), 2.62 (br s, 2H), 1.66 (br d, J = 7.2 |
| | | Hz, 3H), 1.22 (d, J = 6.2 Hz, 6H), 1.16 (d, J = 7.0 Hz, 3H) ppm |
| 326 | 589.3 | 1H NMR (400 MHz, DMSO-d6) δ = 9.63-9.60 (m, 1H), 9.34 (s, 1H), 8.52-8.49 (m, |
| | | 2H), 8.38 (s, 1H), 8.25-8.23 (m, 1H), 8.01 (d, J = 8.8 Hz, 1H), 7.76 (s, 1H), 7.73 (d, |
| | | J = 8.0 Hz, 1H), 6.96-6.81 (m, 3H), 4.97 (s, 2H), 4.81 (br d, J = 5.2 Hz, 2H), 4.23- |
| | | 4.20 (m, 2H), 4.05-4.04(m, 2H), 3.68-3.65 (m, 2H), 3.62 (br d, J = 6.4 Hz, 1H), |
| | | 3.56-3.50 (m, 4H), 3.28 (s, 3H), 1.16 (d, J = 6.4 Hz, 3H) ppm |
| 327 | 561.1 | 1H NMR (400 MHz, DMSO-d6) δ = 9.68-9.65 (m, 1H), 9.45 (s, 1H), 8.78 (d, J = |
| | | 8.8 Hz, 1H), 8.64 (d, J = 8.8 Hz, 1H), 8.54 (d, J = 1.6 Hz, 1H), 8.45 (d, J = 8.0 Hz, |
| | | 1H), 8.33-8.24 (m, 2H), 8.15-7.71 (m, 3H), 4.98 (s, 2H), 4.83 (d, J = 5.6 Hz, 2H), |
| | | 4.29-4.16 (m, 2H), 3.75-3.63 (m, 2H) ppm |
| 328 | 560.3 | 1H NMR (400 MHz, METHANOL-d4) δ = 9.35 (s, 1H), 8.70-8.60 (m, 3H), 8.49 (br |
| | | s, 1H), 8.31 (d, J = 7.4 Hz, 1H), 8.24-8.22 (m, 1H), 7.99 (s, 1H), 7.94-7.92 (m, |
| | | 1H), 7.66 (d, J = 8.0 Hz, 1H), 7.02 (d, J = 8.0 Hz, 1H), 5.90-5.88 (m, 1H), 5.07 (s, |
| | | 2H), 4.95 (s, 2H), 4.40-4.33 (m, 2H), 3.71-3.53 (m, 5H), 3.31-3.26 (m, 1H), 2.93 |
| | | (s, 3H), 2.78-2.65 (m, 1H), 2.39-2.37 (m, 1H) ppm |
| 329 | 541.2 | 1H NMR (400 MHz, DMSO-d6) δ = 9.64-9.62 (m, 1H), 9.43 (s, 1H), 8.65 (d, J = |
| | | 8.6 Hz, 1H), 8.53 (d, J = 1.6 Hz, 1H), 8.45 (d, J = 7.6 Hz, 1H), 8.28-8.18 (m, 2H), |
| | | 7.81 (s, 1H), 7.73 (d, J = 8.0 Hz, 1H), 7.47 (d, J = 7.6 Hz, 1H), 7.13-6.83 (m, 1H), |
| | | 4.98 (s, 2H), 4.82 (br d, J = 6.0 Hz, 2H), 4.27-4.18 (m, 2H), 4.02 (s, 3H), 3.74- |
| | | 3.62 (m, 2H) ppm |
| 330 | 519.2 | 1H NMR (400 MHz, DMSO-d6) δ = 9.68-9.58 (m, 1H), 9.42 (s, 1H), 8.74-8.68 (m, |
| | | 1H), 8.67-8.60 (m, 1H), 8.53 (d, J = 2.0 Hz, 1H), 8.29-8.28 (m, 1H), 8.20 (d, J = |
| | | 6.8 Hz, 1H), 7.93-7.87 (m, 1H), 7.85 (s, 1H), 7.75 (d, J = 8.0 Hz, 1H), 6.97 (d, J = |
| | | 7.6 Hz, 1H), 4.97 (s, 2H), 4.83 (d, J = 6.0 Hz, 2H), 4.54-4.48 (m, 2H), 4.31-4.27 |
| | | (m, 1H), 4.04-4.02 (m, 1H), 3.70-3.60 (m, 1H), 1.43-1.39 (m, 3H), 1.17 (d, J = |
| | | 7.2 Hz, 3H) ppm |
| 331 | 515.1 | 1H NMR (400 MHz, DMSO-d6) δ = 9.65-9.63 (m, 1H), 9.41 (s, 1H), 8.70-8.67 (m, |
| | | 1H), 8.64-8.59 (m, 1H), 8.53 (s, 1H), 8.34 (d, J = 7.6 Hz, 1H), 8.28 (d, J = 7.6 Hz, |
| | | 1H), 7.87-7.82 (m, 2H), 7.74 (d, J = 7.6 Hz, 1H), 7.46 (d, J = 7.6 Hz, 1H), 4.97 (s, |
| | | 2H), 4.82 (d, J = 5.6 Hz, 2H), 4.29 (d, J = 13.2 Hz, 1H), 4.04-3.99 (m, 1H), 3.66- |
| | | 3.57 (m, 1H), 2.24-2.22 (m, 1H), 1.17 (d, J = 6.8 Hz, 3H), 1.11-1.09 (m, 2H), 1.05- |
| | | 1.03 (m, 2H) ppm |
| 332 | 578.2 | 1H NMR (400 MHz, DMSO-d6) δ = 9.63-9.61 (m, 1H), 9.40 (s, 1H), 8.67 (s, 2H), |
| | | 8.54 (d, J = 1.6 Hz, 1H), 8.45 (br s, 1H), 8.27-8.25 (m, 1H), 7.87 (d, J = 7.4 Hz, |
| | | 1H), 7.81 (s, 1H), 7.77-7.69 (m, 2H), 6.68 (d, J = 8.4 Hz, 1H), 5.56- |
| | | 5.33 (m, 1H), 4.98 (s, 2H), 4.82 (br d, J = 5.6 Hz, 2H), 4.26-4.21 (m, 2H), 4.21- |
| | | 4.12 (m, 1H), 4.04-3.96 (m, 1H), 3.96-3.87 (m, 1H), 3.86-3.78 (m, 1H), 3.77- |
| | | 3.63 (m, 3H), 3.44 (s, 3H) ppm |
| 333 | 578.2 | 1H NMR (400 MHz, DMSO-d6) δ = 9.63-9.61 (m, 1H), 9.40 (s, 1H), 8.67 (s, 2H), |
| | | 8.54 (d, J = 1.6 Hz, 1H), 8.44 (s, 1H), 8.27-8.25 (m, 1H), 7.87 (d, J = 7.4 Hz, 1H), |
| | | 7.81 (s, 1H), 7.77-7.69 (m, 2H), 6.68 (d, J = 8.4 Hz, 1H), 5.54-5.35 |
| | | (m, 1H), 4.98 (s, 2H), 4.85-4.78 (m, 2H), 4.26-4.22 (m, 2H), 4.21-4.11 (m, 1H), |
| | | 4.04-3.96 (m, 1H), 3.96-3.87 (m, 1H), 3.87-3.77 (m, 1H), 3.76-3.65 (m, 3H), |
| | | 3.44 (s, 3H) ppm |
| 334 | 602.3 | 1H NMR (400 MHz, DMSO-d6) δ = 9.71-9.57 (m, 1H), 9.38 (s, 1H), 8.72-8.50 (m, |
| | | 3H), 8.44 (br d, J = 1.8 Hz, 1H), 8.27 (d, J = 8.0 Hz, 1H), 7.88-7.79 (m, 2H), 7.78- |
| | | 7.65 (m, 2H), 6.83 (d, J = 8.4 Hz, 1H), 4.98 (s, 2H), 4.82 (br d, J = 5.6 Hz, 2H), 4.74- |
| | | 4.66 (m, 1H), 4.26-4.19 (m, 2H), 3.88-3.78 (m, 2H), 3.68-3.66 (m, 2H), 3.21 (s, |
| | | 3H), 2.01-1.90 (m, 2H), 1.67-1.50 (m, 2H), 1.19-1.05 (m, 6H) ppm |
| 335 | 558.2 | 1H NMR (400 MHz, DMSO-d6) δ = 9.63-9.61 (m, 1H), 9.42 (s, 1H), 8.61 (d, J = |
| | | 8.6 Hz, 1H), 8.52 (d, J = 1.6 Hz, 1H), 8.25-8.23 (m, 1H), 8.07 (d, J = 8.6 Hz, 1H), |
| | | 7.96 (d, J = 8.0 Hz, 1H), 7.81 (s, 1H), 7.73 (d, J = 7.8 Hz, 1H), 7.54- |
| | | 7.41 (m, 2H), 4.97 (s, 2H), 4.82 (d, J = 5.6 Hz, 2H), 4.27-4.18 (m, 2H), 3.95 (s, |
| | | 3H), 3.72-3.63 (m, 2H) ppm |
| 336 | 558.2 | 1H NMR (400 MHz, DMSO-d6) δ = 9.69-9.58 (m, 1H), 9.46 (s, 1H), 8.74 (d, J = |
| | | 8.8 Hz, 1H), 8.53 (d, J = 1.6 Hz, 1H), 8.47-8.41 (m, 1H), 8.36 (d, J = 8.4 Hz, 1H), |
| | | 8.30-8.21 (m, 1H), 7.92 (s, 1H), 7.81 (d, J = 5.2 Hz, 1H), 7.77-7.68 (m, 2H), 7.18 |
| | | (d, J = 3.6 Hz, 1H), 4.98 (s, 2H), 4.85 (d, J = 5.2 Hz, 2H), 4.60-4.43 (m, 2H), 4.30- |
| | | 4.17 (m, 2H), 3.79-3.72 (m, 2H), 3.71-3.63 (m, 2H), 3.24 (s, 3H) ppm |
| 337 | 544.2 | 1H NMR (400 MHz, METHANOL-d4) δ = 9.30 (s, 1H), 8.71-8.53 (m, 3H), 8.23- |
| | | 8.20 (m, 1H), 7.96 (s, 1H), 7.89 (d, J = 7.2 Hz, 1H), 7.74-7.61 (m, 2H), 7.17 (d, J = |
| | | 8.4 Hz, 1H), 5.07-5.03 (m, 2H), 4.94-4.92 (m, 2H), 4.34-4.33 (m, 2H), 3.89 (d, |
| | | J = 6.8 Hz, 2H), 3.56-3.50 (m, 2H), 2.65-2.59 (m, 1H), 1.25-1.22 (m, 3H), 0.97- |
| | | 0.95 (m, 2H), 0.70-0.69 (m, 2H) ppm |
| 338 | 562.2 | 1H NMR (400 MHz, METHANOL-d4) δ = 9.29 (s, 1H), 8.67 (d, J = 8.8 Hz, 1H), 8.56 |
| | | (d, J = 8.8 Hz, 1H), 8.47 (s, 1H), 8.05-8.02 (m, 1H), 7.96 (s, 1H), 7.89 (d, J = 7.2 |
| | | Hz, 1H), 7.71-7.69 (m, 1H), 7.17 (d, J = 8.4 Hz, 1H), 5.14 (s, 2H), 4.92 (s, 2H), |
| | | 4.34-4.32 (m, 2H), 3.91-3.86 (m, 2H), 3.61-3.54 (m, 2H), 2.65-2.58 (m, 1H), |
| | | 1.25-1.21 (m, 3H), 0.99-0.92 (m, 2H), 0.73-0.67 (m, 2H) ppm |
| 339 | 578.2 | 1H NMR (400 MHz, DMSO-d6) δ = 9.63-9.61 (m, 1H), 9.40 (s, 1H), 8.67 (s, 2H), |
| | | 8.54 (d, J = 1.6 Hz, 1H), 8.27-8.25 (m, 1H), 7.87 (d, J = 7.2 Hz, 1H), 7.81 (s, 1H), |
| | | 7.79-7.68 (m, 2H), 6.71 (d, J = 8.2 Hz, 1H), 5.50-5.27 (m, 1H), 4.98 (s, 2H), 4.82 |
| | | (br d, J = 6.0 Hz, 2H), 4.27-4.15 (m, 3H), 3.95-3.75 (m, 2H), 3.73 (br s, 2H), 3.71- |
| | | 3.66 (m, 2H), 3.40 (s, 3H) ppm |
| 340 | 578.2 | 1H NMR (400 MHz, DMSO-d6) δ = 9.62-9.60 (m, 1H), 9.39 (s, 1H), 8.71-8.64 (m, |
| | | 2H), 8.54 (d, J = 1.6 Hz, 1H), 8.26-8.24 (m, 1H), 7.86 (d, J = 7.2 Hz, 1H), 7.81 (s, |
| | | 1H), 7.77-7.68 (m, 2H), 6.70 (d, J = 8.4 Hz, 1H), 5.52-5.25 (m, |
| | | 1H), 4.98 (s, 2H), 4.82 (br d, J = 5.6 Hz, 2H), 4.28-4.14 (m, 3H), 3.94-3.75 (m, |
| | | 2H), 3.72 (br s, 2H), 3.70-3.66 (m, 2H), 3.40 (s, 3H) ppm |
| 341 | 596.2 | 1H NMR (400 MHz, DMSO-d6) δ = 9.63-9.61 (m, 1H), 9.39 (s, 1H), 8.71-8.65 (m, |
| | | 1H), 8.63-8.56 (m, 1H), 8.54 (d, J = 1.6 Hz, 1H), 8.26-8.24 (m, 1H), 7.92 (d, J = |
| | | 7.4 Hz, 1H), 7.81 (s, 1H), 7.79-7.71 (m, 2H), 7.06 (d, J = 8.6 Hz, |
| | | 1H), 4.98 (s, 2H), 4.82 (br d, J = 5.6 Hz, 2H), 4.39 (br t, J = 12.8 Hz, 2H), 4.29- |
| | | 4.17 (m, 2H), 4.07-3.90 (m, 4H), 3.84-3.82 (m, 2H), 3.74-3.63 (m, 2H) ppm |
| 342 | 560.2 | 1H NMR (400 MHz, DMSO-d6) δ = 9.66-9.64 (m, 1H), 9.43 (s, 1H), 8.76-8.69 (m, |
| | | 1H), 8.67-8.60 (m, 1H), 8.55 (d, J = 2.0 Hz, 1H), 8.28-8.26 (m, 1H), 8.22 (d, J = |
| | | 7.6 Hz, 1H), 8.16 (s, 1H), 7.91-7.90 (m, 1H), 7.84 (s, 1H), 7.75 (d, J = 7.8 Hz, 1H), |
| | | 6.99 (d, J = 8.4 Hz, 1H), 5.65-5.56 (m, 1H), 4.99 (s, 2H), 4.83 (d, J = 5.6 Hz, 2H), |
| | | 4.24-4.22 (m, 2H), 3.74-3.66 (m, 2H), 3.07-3.05 (m, 1H), 2.84 (br d, J = 8.0 Hz, |
| | | 2H), 2.61-2.54 (m, 1H), 2.48-2.43 (m, 1H), 2.39 (s, 3H), 2.01-1.89 (m, 1H) ppm |
| 343 | 540.2 | 1H NMR (400 MHz, DMSO-d6) δ = 9.65-9.62 (m, 1H), 9.44 (s, 1H), 8.67 (d, J = |
| | | 8.8 Hz, 1H), 8.52 (d, J = 1.2 Hz, 1H), 8.25-8.23 (m, 1H), 8.06 (d, J = 8.8 Hz, 1H), |
| | | 7.95 (d, J = 7.6 Hz, 1H), 7.83 (s, 1H), 7.73 (d, J = 7.6 Hz, 2H), 7.46-7.42 (m, 1H), |
| | | 7.40-7.08 (m, 1H), 4.97 (s, 2H), 4.82 (d, J = 5.6 Hz, 2H), 4.25-4.18 (m, 2H), 3.71- |
| | | 3.64 (m, 2H), 3.44 (s, 3H) ppm |
| 344 | 602.3 | 1H NMR (400 MHz, DMSO-d6) δ = 9.56-9.54 (m, 1H), 9.40 (s, 1H), 8.72-8.58 (m, |
| | | 2H), 8.47 (d, J = 1.8 Hz, 1H), 8.19-8.17 (m, 1H), 7.91 (d, J = 7.6 Hz, 1H), 7.80 (s, |
| | | 1H), 7.78-7.67 (m, 2H), 7.03 (d, J = 8.4 Hz, 1H), 4.81 (br d, J = 6.0 Hz, 2H), 4.31 |
| | | (br d, J = 11.2 Hz, 2H), 3.73-3.61 (m, 3H), 3.46-3.40 (m, 4H), 3.24 (s, 3H), 2.52- |
| | | 2.52 (m, 2H), 2.31-2.24 (m, 2H), 1.21 (d, J = 6.2 Hz, 6H) ppm |
| 345 | 576.2 | 1H NMR (400 MHz, METHANOL-d4) δ = 9.32 (s, 1H), 8.69-8.64 (m, 2H), 8.62- |
| | | 8.57 (m, 1H), 8.47-8.41 (m, 1H), 8.24-8.22 (m, 1H), 7.98 (s, 1H), 7.83 (d, J = 7.4 |
| | | Hz, 1H), 7.71-7.64 (m, 2H), 6.81 (d, J = 8.6 Hz, 1H), 5.07 (s, 2H), 4.95 (s, 2H), |
| | | 4.39-4.33 (m, 2H), 3.84-3.66 (m, 4H), 3.61-3.53 (m, 3H), 3.39 (s, 3H), 1.29- |
| | | 1.23 (m, 6H) ppm |
| 346 | 545.2 | 1H NMR (400 MHz, DMSO-d6) δ = 9.65-9.63 (m, 1H), 9.42 (s, 1H), 8.75-8.69 (m, |
| | | 1H), 8.65-8.59 (m, 1H), 8.54 (d, J = 1.8 Hz, 1H), 8.48-8.45 (m, 1H), 8.38 (d, J = |
| | | 7.8 Hz, 1H), 8.27-8.25 (m, 1H), 7.90-7.81 (m, 2H), 7.74 (d, J = 7.8 Hz, 1H), 7.57 |
| | | (d, J = 7.6 Hz, 1H), 4.98 (s, 2H), 4.83 (d, J = 6.0 Hz, 2H), 4.27-4.16 (m, 2H), 3.72- |
| | | 3.64 (m, 2H), 3.55-3.52 (m, 1H), 3.40 (br d, J = 2.4 Hz, 1H), 2.94 (s, 3H), 2.47- |
| | | 2.42 (m, 1H), 1.66-1.55 (m, 1H), 1.51-1.43 (m, 1H), 1.23-1.14 (m, 1H) ppm |
| 347 | 577.3 | 1H NMR (400 MHz, DMSO-d6) δ = 9.69-9.58 (m, 1H), 9.39 (s, 1H), 8.56 (d, J = |
| | | 8.8 Hz, 1H), 8.52 (d, J = 1.6 Hz, 1H), 8.46-8.39 (m, 1H), 8.30-8.19 (m, 1H), 7.96 |
| | | (d, J = 8.4 Hz, 1H), 7.79 (s, 1H), 7.72 (d, J = 8.0 Hz, 1H), 7.30-7.20 (m, 1H), 7.19- |
| | | 7.08 (m, 2H), 4.97 (s, 2H), 4.81 (br d, J = 5.6 Hz, 2H), 4.28-4.14 (m, 2H), 3.76- |
| | | 3.62 (m, 2H), 3.55-3.48 (m, 5H), 3.31-3.30 (m, 2H), 3.23 (s, 3H), 2.86 (s, 3H) ppm |
| 348 | 596.2 | 1H NMR (400 MHz, DMSO-d6) δ = 9.71-9.68 (m, 1H), 9.29 (s, 1H), 8.63 (s, 1H), |
| | | 8.62-8.54 (m, 2H), 8.45 (d, J = 8.0 Hz, 1H), 8.10 (d, J = 8.0 Hz, 1H), 7.91 (d, J = |
| | | 8.8 Hz, 1H), 7.85-7.33 (m, 3H), 5.63-5.27 (m, 1H), 5.23-4.99 (m, 1H), 4.80 (d, |
| | | J = 6.0 Hz, 2H), 3.38 (s, 3H), 2.86-2.70 (m, 4H) ppm |
| 349 | 574.3 | 1H NMR (400 MHz, METHANOL-d4) δ = 9.28 (s, 1H), 8.66-8.59 (m, 2H), 8.58- |
| | | 8.51 (m, 1H), 8.47-8.41 (m, 1H), 8.22-8.20 (m, 1H), 7.95 (s, 1H), 7.90 (d, J = 7.2 |
| | | Hz, 1H), 7.74-7.67 (m, 1H), 7.64 (d, J = 8.0 Hz, 1H), 7.20 (d, J = 8.4 Hz, 1H), 5.07- |
| | | 5.02 (m, 2H), 4.92 (s, 2H), 4.37-4.30 (m, 2H), 4.00-3.97 (m, 2H), 3.72-3.69 (m, |
| | | 2H), 3.55-3.51 (m, 2H), 3.35 (s, 3H), 2.71-2.64 (m, 1H), 1.00-0.91 (m, 2H), 0.77- |
| | | 0.70 (m, 2H) ppm |
| 350 | 529.2 | 1H NMR (400 MHz, DMSO-d6) δ = 9.55-9.53 (m, 1H), 8.95 (s, 1H), 8.51 (d, J = 1.6 |
| | | Hz, 1H), 8.29 (br s, 1H), 8.25-8.23 (m, 1H), 8.11 (d, J = 9.2 Hz, 1H), 7.73 (d, J = 8.0 |
| | | Hz, 1H), 7.36-7.33 (m, 2H), 7.25 (d, J = 8.4 Hz, 1H), 7.05 (s, 1H), 7.00 (d, J = 8.4 |
| | | Hz, 1H), 4.97 (s, 2H), 4.69 (d, J = 6.0 Hz, 2H), 4.23-4.21 (m, 2H), 3.99-3.95 (m, |
| | | 2H), 3.69-3.66 (m, 2H), 2.692.66 (m, 2H), 2.27 (s, 3H), 1.92-1.86 (m, 2H) ppm |
| 351 | 628.3 | 1H NMR (400 MHz, DMSO-d6) δ = 9.63-9.60 (m, 1H), 9.41 (s, 1H), 8.71-8.60 (m, |
| | | 2H), 8.43 (d, J = 1.8 Hz, 1H), 8.27 (d, J = 1.8 Hz, 1H), 8.13 (s, 1H), 7.91 (d, J = 7.4 |
| | | Hz, 1H), 7.82 (s, 1H), 7.78-7.70 (m, 1H), 7.03 (d, J = 8.6 Hz, 1H), 5.32 (br s, 2H), |
| | | 4.87-4.77 (m, 2H), 4.37-4.28 (m, 2H), 4.27-4.21 (m, 2H), 3.71-3.63 (m, 4H), |
| | | 2.55-2.54 (m, 2H), 1.33 (s, 3H), 1.21 (d, J = 6.2 Hz, 6H), 0.88 (s, 4H) ppm |
| 352 | 588.3 | 1H NMR (400 MHz, METHANOL-d4) δ = 9.30 (s, 1H), 8.69-8.61 (m, 2H), 8.61- |
| | | 8.55 (m, 1H), 8.52-8.45 (m, 1H), 8.23-8.20 (m, 1H), 7.96 (s, 1H), 7.86 (d, J = 7.2 |
| | | Hz, 1H), 7.73-7.67 (m, 1H), 7.64 (d, J = 8.0 Hz, 1H), 6.97 (d, J = 8.4 Hz, 1H), 5.05 |
| | | (s, 2H), 4.93 (s, 2H), 4.50-4.43 (m, 1H), 4.38-4.30 (m, 3H), 3.56-3.51 (m, 2H), |
| | | 3.42 (s, 3H), 3.14-3.03 (m, 2H), 2.80-2.77 (m, 1H), 2.26-2.15 (m, 1H), 1.77- |
| | | 1.64 (m, 1H), 1.53-1.39 (m, 1H), 1.08 (d, J = 6.8 Hz, 3H) ppm |
| 353 | 588.3 | 1H NMR (400 MHz, METHANOL-d4) δ = 9.30 (s, 1H), 8.69-8.61 (m, 2H), 8.61- |
| | | 8.55 (m, 1H), 8.52-8.44 (m, 1H), 8.23-8.21 (m, 1H), 7.97 (s, 1H), 7.86 (d, J = 7.2 |
| | | Hz, 1H), 7.73-7.67 (m, 1H), 7.64 (d, J = 8.0 Hz, 1H), 6.97 (d, J = 8.4 Hz, 1H), 5.05 |
| | | (s, 2H), 4.93 (s, 2H), 4.50-4.43 (m, 1H), 4.38-4.31 (m, 3H), 3.56-3.51 (m, 2H), |
| | | 3.42 (s, 3H), 3.14-3.03 (m, 2H), 2.80-2.76 (m, 1H), 2.26-2.15 (m, 1H), 1.77- |
| | | 1.65 (m, 1H), 1.53-1.39 (m, 1H), 1.08 (d, J = 6.8 Hz, 3H) ppm |
| 354 | 588.3 | 1H NMR (400 MHz, DMSO-d6) δ = 9.49-9.46 (m, 1H), 9.39 (s, 1H), 8.72-8.56 (m, |
| | | 2H), 8.29 (d, J = 2.1 Hz, 1H), 8.20 (d, J = 1.8 Hz, 1H), 7.90 (d, J = 7.4 Hz, 1H), 7.80- |
| | | 7.68 (m, 2H), 7.03 (d, J = 8.4 Hz, 1H), 4.79 (d, J = 5.6 Hz, 2H), 4.36-4.17 (m, |
| | | 4H), 3.74-3.62 (m, 2H), 3.60-3.52 (m, 2H), 2.52 (br s, 2H), 2.35-2.26 (m, 5H), |
| | | 1.21 (d, J = 6.2 Hz, 6H) ppm |
| 355 | 624.2 | 1H NMR (400 MHz, DMSO-d6) δ = 9.89-9.67 (m, 1H), 9.41 (s, 1H), 8.80-8.59 (m, |
| | | 3H), 8.53 (s, 1H), 8.46 (br s, 1H), 7.95-7.89 (m, 1H), 7.87-7.82 (m, 1H), 7.75- |
| | | 7.73 (m, 1H), 7.65-7.36 (m, 1H), 7.04 (d, J = 8.6 Hz, 1H), 5.18-5.01 (m, 2H), 4.88- |
| | | 4.79 (m, 2H), 4.32 (br d, J = 11.4 Hz, 2H), 4.27-4.21 (m, 2H), 3.83-3.74 (m, 2H), |
| | | 3.73-3.64 (m, 2H), 2.56-2.55 (m, 2H), 1.22 (d, J = 6.2 Hz, 6H) ppm |
| 356 | 515.2 | 1H NMR (400 MHz, DMSO-d6) δ = 9.60-9.58 (m, 1H), 9.38 (s, 1H), 8.71 (d, J = |
| | | 8.8 Hz, 1H), 8.51 (d, J = 1.6 Hz, 1H), 8.48-8.45 (m, 1H), 8.23-8.21 (m, 1H), 8.13 |
| | | (d, J = 9.2 Hz, 1H), 7.99 (d, J = 8.0 Hz, 1H), 7.80 (s, 1H), 7.72 (d, J = |
| | | 7.8 Hz, 1H), 7.39 (d, J = 6.8 Hz, 1H), 7.36-7.28 (m, 1H), 7.22-7.12 (m, 1H), 4.97 |
| | | (s, 2H), 4.88-4.76 (m, 2H), 4.28-4.15 (m, 2H), 3.91 (s, 2H), 3.73-3.60 (m, 2H) ppm |
| 357 | 566.3 | 1H NMR (400 MHz, CHLOROFORM-d) δ = 9.52-9.25 (m, 1H), 8.62 (s, 1H), 8.48- |
| | | 8.22 (m, 4H), 8.18 (br s, 1H), 8.07 (d, J = 8.0 Hz, 1H), 7.61 (s, 1H), 7.50 (br d, J = |
| | | 7.4 Hz, 1H), 7.33 (br d, J = 8.0 Hz, 1H), 6.93-6.58 (m, 1H), 5.14-5.00 (m, 4H), |
| | | 4.44-4.35 (m, 2H), 3.96-3.89 (m, 1H), 3.46-3.38 (m, 2H), 0.95-0.87 (m, 2H), |
| | | 0.83 (br s, 2H) ppm |
| 358 | 598.2 | 1H NMR (400 MHz, DMSO-d6) δ = 9.62-9.60 (m, 1H), 9.38 (s, 1H), 8.68 (d, J = |
| | | 8.4 Hz, 1H), 8.53-8.51 (m, 2H), 8.26-8.24 (m, 1H), 7.89 (d, J = 7.6 Hz, 1H), 7.81 |
| | | (s, 1H), 7.78-7.69 (m, 2H), 6.98 (d, J = 8.6 Hz, 1H), 6.47-6.15 (m, 1H), 4.97 (S. |
| | | 2H), 4.81 (d, J = 5.6 Hz, 2H), 4.27-4.19 (m, 2H), 4.10-4.08 (m, 2H), 3.84-3.76 |
| | | (m, 2H), 3.71-3.64 (m, 2H), 3.59-3.57 (m, 2H), 3.27 (s, 3H) ppm |
| 359 | 602.3 | 1H NMR (400 MHz, DMSO-d6) δ = 9.64-9.61 (m, 1H), 9.40 (s, 1H), 8.71-8.58 (m, |
| | | 2H), 8.51 (d, J = 1.6 Hz, 1H), 8.46 (d, J = 3.6 Hz, 1H), 8.28-8.25 (m, 1H), 7.92 (d, |
| | | J = 7.2 Hz, 1H), 7.83 (s, 1H), 7.78-7.71 (m, 2H), 7.03 (d, J = 8.4 Hz, 1H), 5.02- |
| | | 4.90 (m, 2H), 4.82 (d, J = 3.6 Hz, 2H), 4.38-4.27 (m, 3H), 4.19-4.15 (m, 1H), 3.72- |
| | | 3.63 (m, 2H), 3.46-3.41 (m, 1H), 2.52 (s, 2H), 1.76-1.65 (m, 1H), 1.36-1.25 (m, |
| | | 1H), 1.21 (d, J = 6.4 Hz, 6H), 0.97-0.94 (m, 3H) ppm |
| 360 | 602.3 | 1H NMR (400 MHz, DMSO-d6) δ = 9.64-9.61 (m, 1H), 9.40 (s, 1H), 8.71-8.58 (m, |
| | | 2H), 8.51 (d, J = 1.6 Hz, 1H), 8.42 (s, 1H), 8.28-8.25 (m, 1H), 7.92 (d, J = 7.2 Hz, |
| | | 1H), 7.83 (s, 1H), 7.78-7.68 (m, 2H), 7.03 (d, J = 8.4 Hz, 1H), 5.05-4.91 (m, 2H), |
| | | 4.81 (d, J = 4.8 Hz, 2H), 4.31 (d, J = 12.0 Hz, 3H), 4.19-4.15 (m, 1H), 3.74-3.61 |
| | | (m, 2H), 3.46-3.41 (m, 1H), 2.48-2.42 (m, 2H), 1.81-1.64 (m, 1H), 1.35-1.26 |
| | | (m, 1H), 1.21 (d, J = 6.4 Hz, 6H), 0.97-0.94 (m, 3H) ppm |
| 361 | 574.5 | 1H NMR (400 MHz, METHANOL-d4) δ = 9.30 (s, 1H), 8.70-8.65 (m, 1H), 8.63 (d, |
| | | J = 2.0 Hz, 1H), 8.57 (d, J = 8.4 Hz, 1H), 8.44 (s, 1H), 8.21-8.20 (m, 1H), 7.96 (s, |
| | | 1H), 7.83 (d, J = 7.6 Hz, 1H), 7.70-7.67 (m, 1H), 7.64 (d, J = 8.0 Hz, 1H), 6.77 (d, |
| | | J = 8.4 Hz, 1H), 5.05 (s, 2H), 4.93 (s, 2H), 4.37-4.31 (m, 2H), 3.91-3.84 (m, 2H), |
| | | 3.83-3.77 (m, 2H), 3.57-3.50 (m, 2H), 3.37-3.34 (m, 1H), 3.17 (s, 3H), 0.53- |
| | | 0.48 (m, 2H), 0.47-0.42 (m, 2H) ppm |
| 362 | 572.3 | 1H NMR (400 MHz, DMSO-d6) δ = 9.58-9.56 (m, 1H), 9.40 (s, 1H), 8.71-8.58 (m, |
| | | 2H), 8.46 (d, J = 1.6 Hz, 1H), 8.43-8.39 (m, 1H), 8.14-8.12 (m, 1H), 7.94-7.87 |
| | | (m, 1H), 7.79 (s, 1H), 7.77-7.70 (m, 1H), 7.53 (d, J = 8.2 Hz, 1H), |
| | | 7.03 (d, J = 8.4 Hz, 1H), 4.80 (br d, J = 5.6 Hz, 2H), 4.31 (br d, J = 11.8 Hz, 2H), |
| | | 3.74-3.58 (m, 3H), 3.16-3.07 (m, 2H), 2.65-2.57 (m, 2H), 2.31-2.11 (m, 2H), |
| | | 1.36 (d, J = 6.8 Hz, 3H), 1.21 (d, J = 6.2 Hz, 6H) ppm |
| 363 | 572.3 | 1H NMR (400 MHz, DMSO-d6) δ = 9.57-9.56 (M, 1H), 9.39 (s, 1H), 8.73-8.58 (m, |
| | | 2H), 8.45 (d, J = 1.6 Hz, 1H), 8.41 (br s, 1H), 8.14-8.12 (M, 1H), 7.91 (d, J = 7.2 |
| | | Hz, 1H), 7.79 (s, 1H), 7.77-7.70 (m, 1H), 7.53 (d, J = 8.2 Hz, 1H), 7.02 (d, J = 8.4 |
| | | Hz, 1H), 4.80 (br d, J = 5.6 Hz, 2H), 4.31 (br d, J = 11.6 Hz, 2H), 3.71-3.59 (m, |
| | | 3H), 3.15-3.06 (m, 2H), 2.64 (brd, J = 4.0 Hz, 2H), 2.32-2.25 (m, 1H), 2.22-2.08 |
| | | (m, 1H), 1.36 (d, J = 6.8 Hz, 3H), 1.21 (d, J = 6.2 Hz, 6H) ppm |
| 364 | 558.2 | 1H NMR (400 MHz, DMSO-d6) δ = 9.53-9.51 (m, 1H), 9.40 (s, 1H), 8.73-8.55 (m, |
| | | 2H), 8.47-8.37 (m, 1H), 8.24-8.22 (m, 1H), 7.92 (d, J = 7.4 Hz, 1H), 7.82 (s, 1H), |
| | | 7.78-7.71 (m, 1H), 7.68 (d, J = 8.0 Hz, 1H), 7.03 (d, J = 8.6 Hz, |
| | | 1H), 4.81 (brd, J = 5.6 Hz, 2H), 4.31 (br d, J = 12.4 Hz, 2H), 3.82-3.74 (m, 1H), |
| | | 3.72-3.64 (m, 2H), 3.59-3.57 (m, 1H), 3.05-2.95 (m, 1H), 2.48-2.44 (m, 2H), |
| | | 1.39 (d, J = 6.8 Hz, 3H), 1.21 (d, J = 6.2 Hz, 6H) ppm |
| 365 | 560.3 | 1H NMR (400 MHz, DMSO-d6) δ = 9.61-9.59 (m, 1H), 9.40 (s, 1H), 8.74-8.58 (m, |
| | | 2H), 8.49-8.42 (m, 1H), 8.40 (s, 1H), 8.37-8.31 (m, 1H), 8.06-8.04 (m, 1H), 7.91 |
| | | (d, J = 7.4 Hz, 1H), 7.87-7.80 (m, 2H), 7.78-7.69 (m, 1H), 7.03 (d, J = 8.4 Hz, |
| | | 1H), 4.83 (d, J = 5.8 Hz, 2H), 4.35-4.26 (m, 2H), 3.71-3.64 (m, 2H), 3.53-3.48 |
| | | (m, 1H), 2.55-2.52 (m, 3H), 1.20-1.18 (m, 12H) ppm |
| 366 | 584.3 | 1H NMR (400 MHz, DMSO-d6) δ = 9.63-9.61 (m, 1H), 9.40 (s, 1H), 8.58 (d, J = |
| | | 8.8 Hz, 1H), 8.53 (d, J = 1.8 Hz, 1H), 8.26-8.24 (m, 1H), 8.20 (d, J = 8.8 Hz, 1H), |
| | | 7.96 (d, J = 7.8 Hz, 1H), 7.80 (s, 1H), 7.73 (d, J = 7.8 Hz, 1H), 7.41 (s, |
| | | 1H), 7.31 (d, J = 8.0 Hz, 1H), 7.23-6.92 (m, 1H), 4.97 (s, 2H), 4.82 (br d, J = 5.8 |
| | | Hz, 2H), 4.36-4.27 (m, 2H), 4.26-4.14 (m, 2H), 3.75-3.62 (m, 4H), 3.28 (s, 3H) ppm |
| 367 | 518.2 | 1H NMR (400 MHz, DMSO-d6) δ = 9.67-9.51 (m, 1H), 9.11 (s, 1H), 8.53 (d, J = |
| | | 2.0 Hz, 1H), 8.36 (s, 1H), 8.31 (d, J = 2.0 Hz, 2H), 8.28-8.24 (m, 1H), 7.94-7.89 |
| | | (m, 1H), 7.74 (d, J = 8.0 Hz, 1H), 7.51 (s, 1H), 7.38-7.32 (m, 1H), 7.12-7.04 (m, |
| | | 1H), 4.98 (s, 2H), 4.74 (d, J = 5.6 Hz, 2H), 4.35 (s, 4H), 4.28-4.14 (m, 2H), 3.76- |
| | | 3.62 (m, 2H) ppm |
| 368 | 554.2 | 1H NMR (400 MHz, DMSO-d6) δ = 9.73-9.53 (m, 1H), 9.39 (s, 1H), 8.62-8.46 (m, |
| | | 2H), 8.36-8.20 (m, 1H), 8.07 (d, J = 8.4 Hz, 1H), 7.87 (d, J = 8.0 Hz, 1H), 7.80 (s, |
| | | 1H), 7.73 (d, J = 7.6 Hz, 1H), 7.39-7.16 (m, 2H), 4.97 (s, 2H), 4.82 (d, J = 6.0 Hz, |
| | | 2H), 4.34-4.12 (m, 2H), 3.92 (s, 3H), 3.76-3.58 (m, 2H), 2.08 (s, 3H) ppm |
| 369 | 545.2 | 1H NMR (400 MHz, DMSO-d6) δ = 9.64-9.62 (m, 1H), 9.41 (s, 1H), 8.72-8.66 (m, |
| | | 1H), 8.65-8.60 (m, 1H), 8.54 (d, J = 2.0 Hz, 1H), 8.44 (s, 1H), 8.39-8.31 (m, 1H), |
| | | 8.27-8.25 (m, 1H), 7.89-7.80 (m, 2H), 7.74 (d, J = 8.0 Hz, 1H), 7.47 (d, J = 7.8 |
| | | Hz, 1H), 4.98 (s, 2H), 4.82 (d, J = 6.0 Hz, 2H), 4.27-4.20 (m, 2H), 3.72-3.65 (m, |
| | | 2H), 3.493.47 (m, 1H), 3.36-3.36 (m, 1H), 3.27 (s, 3H), 2.20-2.10 (m, 1H), 1.86- |
| | | 1.75 (m, 1H), 1.31-1.30 (m, 1H), 1.05-0.96 (m, 1H) ppm |
| 370 | 574.4 | 1H NMR (400 MHz, METHANOL-d4) δ = 9.30 (s, 1H), 8.70-8.65 (m, 1H), 8.63 (d, |
| | | J = 2.0 Hz, 1H), 8.60-8.57 (m, 1H), 8.23-8.21 (m, 1H), 7.96 (s, 1H), 7.83 (d, J = |
| | | 7.2 Hz, 1H), 7.70-7.63 (m, 2H), 6.79 (d, J = 8.4 Hz, 1H), 5.05 (s, 2H), 4.95-4.90 |
| | | (m, 2H), 4.84-4.80 (m, 2H), 4.64-4.61 (m, 2H), 4.36-4.33 (m, 2H), 4.05 (d, J = |
| | | 7.2 Hz, 2H), 3.65-3.60 (m, 2H), 3.56-3.50 (m, 3H), 1.25-1.18 (m, 3H) ppm |
| 371 | 558.3 | 1H NMR (400 MHz, DMSO-d6) δ = 9.63-9.61 (m, 1H), 9.45 (s, 1H), 8.82-8.60 (m, |
| | | 2H), 8.49 (s, 1H), 8.33 (d, J = 8.0 Hz, 1H), 8.18-8.05 (m, 1H), 7.97-7.79 (m, 3H), |
| | | 7.75-7.73 (m, 1H), 7.04 (d, J = 8.6 Hz, 1H), 4.85 (br d, J = 5.6 Hz, 2H), 4.32 (brd, |
| | | J = 12.0 Hz, 2H), 3.72-3.65 (m, 2H), 2.99-2.90 (m, 1H), 2.54 (br s, 2H), 1.27- |
| | | 1.11 (m, 8H), 1.10-1.04 (m, 2H) ppm |
| 372 | 576.3 | 1H NMR (400 MHz, DMSO-d6) δ = 9.63-9.61 (m, 1H), 9.38 (s, 1H), 8.67 (d, J = |
| | | 8.6 Hz, 1H), 8.61-8.50 (m, 2H), 8.43 (br d, J = 3.4 Hz, 1H), 8.27-8.25 (m, 1H), |
| | | 7.82-7.77 (m, 2H), 7.74 (d, J = 8.0 Hz, 1H), 7.70-7.62 (m, 1H), 6.78 (d, J = 8.6 |
| | | Hz, 1H), 4.98 (s, 3H), 4.82 (d, J = 6.0 Hz, 2H), 4.23-4.21 m, 2H), 3.72-3.62 (m, |
| | | 2H), 3.57-3.41 (m, 4H), 3.26 (s, 3H), 1.23-1.16 (m, 6H) ppm |
| 373 | 612.2 | 1H NMR (400 MHz, DMSO-d6) δ = 9.68-9.56 (m, 1H), 9.39 (s, 1H), 8.69 (d, J = |
| | | 8.4 Hz, 1H), 8.62-8.51 (m, 2H), 8.40-8.32 (m, 1H), 8.29-8.20 (m, 1H), 7.89 (d, |
| | | J = 7.2 Hz, 1H), 7.81 (s, 1H), 7.77-7.70 (m, 2H), 7.00 (d, J = 8.8 Hz, 1H), 4.98 (s, |
| | | 2H), 4.82 (d, J = 5.6 Hz, 2H), 4.28-4.19 (m, 4H), 3.86-3.80 (m, 2H), 3.70-3.67 |
| | | (m, 2H), 3.63-3.60 (m, 2H), 3.27 (s, 3H), 1.72-1.59 (m, 3H) ppm |
| 374 | 588.3 | 1H NMR (400 MHz, METHANOL-d4) δ = 9.30 (s, 1H), 8.69-8.65 (m, 1H), 8.63 (d, |
| | | J = 1.8 Hz, 1H), 8.60-8.56 (m, 1H), 8.22-8.20 (m, 1H), 7.96 (s, 1H), 7.86 (d, J = |
| | | 7.4 Hz, 1H), 7.70-7.61 (m, 2H), 6.79 (d, J = 8.6 Hz, 1H), 5.05 (s, 2H), 4.93 (s, 2H), |
| | | 4.56-4.46 (m, 1H), 4.37-4.31 (m, 2H), 3.92-3.84 (m, 2H), 3.62-3.60 (m, 2H), |
| | | 3.56-3.50 (m, 2H), 3.39 (s, 3H), 2.46-2.35 (m, 2H), 2.30-2.16 (m, 2H), 1.85- |
| | | 1.76 (m, 2H) ppm |
| 375 | 545.2 | 1H NMR (400 MHz, DMSO-d6) δ = 9.65-9.63 (m, 1H), 9.42 (s, 1H), 8.75-8.68 (m, |
| | | 1H), 8.66-8.59 (m, 1H), 8.54 (d, J = 1.6 Hz, 1H), 8.27-8.25 (m, 1H), 8.22-8.15 |
| | | (m, 1H), 7.94-7.86 (m, 1H), 7.83 (s, 1H), 7.74 (d, J = 7.6 Hz, 1H), 7.05-6.89 (m, |
| | | 1H), 4.98 (s, 2H), 4.83 (d, J = 5.6 Hz, 2H), 4.44 (d, J = 6.8 Hz, 2H), 4.24-4.21 (m, |
| | | 2H), 3.72-3.64 (m, 2H), 2.88-2.73 (m, 1H), 2.15-2.05 (m, 2H), 1.96-1.84 (m, 4H) ppm |
| 376 | 531.2 | 1H NMR (400 MHz, DMSO-d6) δ = 9.66-9.64 (m, 1H), 9.43 (s, 1H), 8.76-8.66 (m, |
| | | 2H), 8.55 (d, J = 1.8 Hz, 1H), 8.46 (d, J = 7.8 Hz, 1H), 8.37 (br s, 1H), 8.28-8.26 |
| | | (m, 1H), 7.95-7.93 (m, 1H), 7.85 (s, 1H), 7.75 (d, J = 8.0 Hz, 1H), |
| | | 7.51 (d, J = 7.8 Hz, 1H), 4.99 (s, 2H), 4.84 (d, J = 5.8 Hz, 2H), 4.28-4.20 (m, 2H), |
| | | 4.17-4.15 (m, 1H), 4.02-4.00 (m, 1H), 3.93-3.84 (m, 2H), 3.75-3.65 (m, 3H), |
| | | 2.41-2.35 (m, 1H), 2.31-2.23 (m, 1H) ppm |
| 377 | 557.2 | 1H NMR (400 MHz, DMSO-d6) δ = 9.67-9.64 (m, 1H), 9.42 (s, 1H), 8.74-8.63 (m, |
| | | 2H), 8.54 (d, J = 2.0 Hz, 1H), 8.47 (s, 1H), 8.37 (d, J = 7.2 Hz, 1H), 8.27 (d, J = 7.6 |
| | | Hz, 1H), 7.92 (d, J = 8.0 Hz, 1H), 7.83 (s, 1H), 7.74 (d, J = 8.0 Hz, 1H), 7.50 (d, J = |
| | | 7.2 Hz, 1H), 4.98 (s, 2H), 4.83 (d, J = 5.6 Hz, 2H), 4.28-4.19 (m, 2H), 4.02-3.94 |
| | | (m, 1H), 3.91-3.83 (m, 1H), 3.72-3.65 (m, 2H), 3.63-3.55 (m, 1H), 3.48-3.43 |
| | | (m, 1H), 2.74-2.68 (m, 1H), 2.13-2.08 (m, 1H), 1.94-1.85 (m, 1H), 1.45-1.38 |
| | | (m, 1H), 1.10 (d, J = 4.0 Hz, 1H) ppm |
| 378 | 557.2 | 1H NMR (400 MHz, DMSO-d6) δ = 9.67-9.64 (m, 1H), 9.42 (s, 1H), 8.74-8.63 (m, |
| | | 2H), 8.54 (d, J = 2.0 Hz, 1H), 8.47 (s, 1H), 8.36 (d, J = 7.2 Hz, 1H), 8.27 (d, J = 7.6 |
| | | Hz, 1H), 7.91 (d, J = 8.0 Hz, 1H), 7.83 (s, 1H), 7.74 (d, J = 8.0 Hz, 1H), 7.50 (d, J = |
| | | 7.2 Hz, 1H), 4.98 (s, 2H), 4.82 (d, J = 5.6 Hz, 2H), 4.28-4.19 (m, 2H), 4.02-3.94 |
| | | (m, 1H), 3.91-3.83 (m, 1H), 3.72-3.65 (m, 2H), 3.63-3.55 (m, 1H), 3.48-3.43 |
| | | (m, 1H), 2.74-2.68 (m, 1H), 2.13-2.08 (m, 1H), 1.94-1.85 (m, 1H), 1.45-1.38 |
| | | (m, 1H), 1.10 (d, J = 4.0 Hz, 1H) ppm |
| 379 | 530.3 | 1H NMR (400 MHz, MeOD) δ = 8.97 (s, 1H), 8.61 (d, J = 2.0 Hz, 1H), 8.21-8.18 (m, |
| | | 1H), 8.11 (d, J = 8.8 Hz, 2H), 7.71 (d, J = 9.2 Hz, 1H), 7.67-7.61 (m, 3H), 7.08-7.05 |
| | | (m, 1H), 5.30-5.26 (m, 1H), 5.04 (s, 2H), 4.85-4.83 (m, 2H), 4.34-4.32 (m, 2H), |
| | | 3.53-3.51 (m, 2H), 2.98-2.93 (m, 1H), 2.86-2.85 (m, 1H), 2.16-2.14 (m, 1H), 1.90- |
| | | 1.85 (m, 1H), 1.35 (d, J = 6.8 Hz, 3H) ppm |
| 380 | 519.2 | 1H NMR (400 MHz, DMSO-d6) δ = 9.50-9.48 (m, 1H), 8.93 (s, 1H), 8.50 (d, J = |
| | | 1.6 Hz, 1H), 8.36 (s, 1H), 8.26-8.14 (m, 3H), 7.71 (d, J = 8.0 Hz, 1H), 7.48-7.36 |
| | | (m, 2H), 4.96 (s, 2H), 4.68 (d, J = 5.6 Hz, 2H), 4.26-4.16 (m, 2H), 4.00-3.90 (m, |
| | | 2H), 3.71 (s, 3H), 3.68-3.64 (m, 2H), 2.77-2.75 (m, 2H), 1.99 (br d, J = 5.0 Hz, 2H) ppm |
| 381 | 531.2 | 1H NMR (400 MHz, DMSO-d6) δ = 9.66-9.63 (m, 1H), 9.42 (s, 1H), 8.71 (d, J = |
| | | 8.8 Hz, 1H), 8.62 (d, J = 8.8 Hz, 1H), 8.54 (d, J = 1.6 Hz, 1H), 8.45 (s, 1H), 8.28- |
| | | 8.26 (m, 1H), 8.20 (d, J = 7.2 Hz, 1H), 7.92-7.88 (m, 1H), 7.83 (s, 1H), 7.75 (d, |
| | | J = 8.0 Hz, 1H), 7.00 (d, J = 7.6 Hz, 1H), 4.99 (s, 2H), 4.83 (d, J = 5.6 Hz, 2H), 4.31 |
| | | (d, J = 7.2 Hz, 2H), 4.27-4.20 (m, 2H), 3.73-3.64 (m, 2H), 1.38-1.28 (m, 1H), |
| | | 0.66-0.53 (m, 2H), 0.48-0.33 (m, 2H) ppm |
| 382 | 560.2 | 1H NMR (400 MHz, DMSO-d6) δ = 9.64-9.62 (m, 1H), 9.38 (s, 1H), 8.70-8.63 (m, |
| | | 1H), 8.60-8.56 (m, 1H), 8.54 (d, J = 2.0 Hz, 1H), 8.27-8.25 (m, 1H), 7.85-7.78 |
| | | (m, 2H), 7.76-7.65 (m, 2H), 6.85 (d, J = 8.4 Hz, 1H), 5.08-4.95 (m, 3H), 4.82 (d, |
| | | J = 5.6 Hz, 2H), 4.58-4.43 (m, 2H), 4.26-4.19 (m, 2H), 4.01-3.85 (m, 2H), 3.72- |
| | | 3.65 (m, 2H), 3.19 (s, 3H), 2.65-2.60 (m, 2H) ppm |
| 383 | 560.3 | 1H NMR (400 MHz, DMSO-d6) δ = 9.64-9.62 (m, 1H), 9.38 (s, 1H), 8.69-8.63 (m, |
| | | 1H), 8.62-8.52 (m, 2H), 8.27-8.25 (m, 1H), 7.85-7.78 (m, 2H), 7.76-7.67 (m, |
| | | 2H), 6.85 (d, J = 8.4 Hz, 1H), 5.05-5.02 (m, 1H), 4.98 (s, 2H), 4.82 (d, J = 5.2 Hz, |
| | | 2H), 4.56-4.43 (m, 2H), 4.26-4.20 (m, 2H), 4.00-3.86 (m, 2H), 3.71-3.65 (m, |
| | | 2H), 3.19 (s, 3H), 2.65-2.60 (m, 2H) ppm |
| 384 | 541.2 | 1H NMR (400 MHz, DMSO-d6) δ = 9.65-9.63 (m, 1H), 9.44 (s, 1H), 8.81-8.72 (m, |
| | | 1H), 8.66 (d, J = 8.8 Hz, 1H), 8.52 (d, J = 2.0 Hz, 1H), 8.48-8.41 (m, 2H), 8.31- |
| | | 7.89 (m, 3H), 7.86 (s, 1H), 7.76-7.70 (m, 1H), 7.27 (d, J = 8.2 Hz, 1H), 5.00-4.92 |
| | | (m, 2H), 4.83 (br d, J = 5.2 Hz, 2H), 4.35-4.22 (m, 1H), 4.02-4.00 (m, 1H), 3.69- |
| | | 3.58 (m, 1H), 1.16 (d, J = 7.2 Hz, 3H) ppm |
| 386 | 538.2 | 1H NMR (400 MHz, DMSO-d6) δ = 9.67-9.58 (m, 1H), 9.38 (s, 1H), 8.56 (d, J = |
| | | 8.8 Hz, 1H), 8.51 (d, J = 1.2 Hz, 1H), 8.42 (s, 1H), 8.30-8.22 (m, 1H), 8.06 (d, J = |
| | | 8.8 Hz, 1H), 7.86-7.77 (m, 2H), 7.73 (d, J = 8.0 Hz, 1H), 7.31 (d, J = 1.6 Hz, 1H), |
| | | 7.21-7.12 (m, 1H), 4.96 (s, 2H), 4.81 (br d, J = 5.6 Hz, 2H), 4.32-4.24 (m, 1H), |
| | | 4.06-3.97 (m, 1H), 3.90 (s, 3H), 3.70-3.59 (m, 1H), 1.16 (d, J = 7.2 Hz, 3H) ppm |
| 387 | 530.2 | 1H NMR (400 MHz, DMSO-d6) δ = 9.63-9.60 (m, 1H), 9.34 (s, 1H), 8.55-8.46 (m, |
| | | 2H), 8.43 (s, 1H), 8.26-8.24 (m, 1H), 8.06 (d, J = 8.4 Hz, 1H), 7.82-7.67 (m, 3H), |
| | | 6.90 (s, 1H), 6.79 (d, J = 8.0 Hz, 1H), 4.97 (s, 2H), 4.80 (d, J = 5.6 Hz, 2H), 4.29- |
| | | 4.17 (m, 2H), 3.87 (s, 3H), 3.71-3.62 (m, 2H), 2.05-1.95 (m, 1H), 1.06-0.97 (m, |
| | | 2H), 0.86-0.75 (m, 2H) ppm |
| 388 | 559.2 | 1H NMR (400 MHz, DMSO-d6) δ = 9.66-9.65 (m, 1H), 9.46 (s, 1H), 8.78 (d, J = |
| | | 8.8 Hz, 1H), 8.62 (d, J = 7.6 Hz, 1H), 8.55-8.47 (m, 2H), 8.33-8.20 (m, 2H), 7.88 |
| | | (s, 1H), 7.75 (d, J = 8.0 Hz, 1H), 7.48 (d, J = 8.0 Hz, 1H), 4.97 (s, 2H), 4.84 (d, J = |
| | | 5.6 Hz, 2H), 4.31-4.27 (m, 1H), 4.04-3.99 (m, 1H), 3.71-3.59 (m, 1H), 1.17 (d, |
| | | J = 6.8 Hz, 3H) ppm |
| 389 | 608.2 | 1H NMR (400 MHz, DMSO-d6) δ = 9.65-9.63 (m, 1H), 9.40 (s, 1H), 8.71-8.59 (m, |
| | | 2H), 8.45 (d, J = 2.2 Hz, 2H), 8.40 (d, J = 2.2 Hz, 1H), 7.90 (d, J = 7.4 Hz, 1H), 7.81 |
| | | (s, 1H), 7.77-7.71 (m, 1H), 7.03 (d, J = 8.4 Hz, 1H), 4.80 (d, J = 5.6 Hz, 2H), 4.38- |
| | | 4.26 (m, 4H), 3.73-3.62 (m, 4H), 2.52 (br s, 2H), 2.32 (br d, J = 1.8 Hz, 2H), 1.21 |
| | | (d, J = 6.4 Hz, 6H) ppm |
| 390 | 581.2 | 1H NMR (400 MHz, DMSO-d6) δ = 9.74-9.59 (m, 1H), 9.42 (s, 1H), 8.76-8.69 (m, 1H), |
| | | 8.68-8.60 (m, 1H), 8.54 (d, J = 1.6 Hz, 1H), 8.40-8.34 (m, 1H), 8.31-8.16 |
| | | (m, 2H), 7.97-7.87 (m, 1H), 7.83 (s, 1H), 7.74 (d, J = 8.0 Hz, 1H), 7.01 (d, J = 8.0 |
| | | Hz, 1H), 4.98 (s, 2H), 4.83 (br d, J = 5.6 Hz, 2H), 4.54 (d, J = 6.4 Hz, 2H), 4.27- |
| | | 4.18 (m, 2H), 3.77-3.60 (m, 2H), 2.85-2.68 (m, 3H), 2.60-2.54 (m, 2H) ppm |
| 391 | 567.2 | 1H NMR (400 MHz, DMSO-d6) δ = 9.65-9.62 (m, 1H), 9.42 (s, 1H), 8.76-8.68 (m, |
| | | 1H), 8.67-8.60 (m, 1H), 8.54 (d, J = 1.6 Hz, 1H), 8.30-8.21 (m, 2H), 7.95-7.91 |
| | | (m, 1H), 7.83 (s, 1H), 7.74 (d, J = 8.0 Hz, 1H), 7.05 (d, J = 8.4 Hz, 1H), 4.98 (s, 2H), |
| | | 4.83 (d, J = 5.6 Hz, 2H), 4.74-4.59 (m, 1H), 4.44-4.39 (m, 1H), 4.29-4.18 (m, |
| | | 2H), 3.75-3.64 (m, 2H), 2.41-2.33 (m, 1H), 1.78-1.71 (m, 1H), 1.65-1.49 (m, 1H) ppm |
| 392 | 551.2 | 1H NMR (400 MHz, DMSO-d6) δ = 9.64-9.61 (m, 1H), 9.43 (s, 1H), 8.74-8.65 (m, |
| | | 2H), 8.55-8.48 (m, 2H), 8.28-8.26 (m, 1H), 7.99-7.97 (m, 1H), 7.86 (s, 1H), 7.74 |
| | | (d, J = 7.8 Hz, 1H), 7.63 (d, J = 7.6 Hz, 1H), 4.97 (s, 2H), 4.83 (br d, J = 5.6 Hz, |
| | | 2H), 4.29-4.27 (m, 1H), 4.02-3.99 (m, 1H), 3.68-3.62 (m, 1H), 2.52 (br s, 2H), |
| | | 2.14-2.06 (m, 1H), 1.17 (d, J = 7.2 Hz, 3H) ppm |
| 393 | 551.2 | 1H NMR (400 MHz, DMSO-d6) δ = 9.64-9.61 (m, 1H), 9.43 (s, 1H), 8.74-8.65 (m, |
| | | 2H), 8.55-8.48 (m, 2H), 8.28-8.26 (m, 1H), 7.99-7.97 (m, 1H), 7.86 (s, 1H), 7.74 |
| | | (d, J = 7.8 Hz, 1H), 7.64 (d, J = 7.6 Hz, 1H), 4.97 (s, 2H), 4.83 (br d, J = 5.6 Hz, |
| | | 2H), 4.29-4.26 (m, 1H), 4.02-4.00 (m, 1H), 3.68-3.62 (m, 1H), 2.52 (br s, 2H), |
| | | 2.14-2.05 (m, 1H), 1.17 (d, J = 7.2 Hz, 3H) ppm |
| 394 | 556.2 | 1H NMR (400 MHz, DMSO-d6) δ = 9.57-9.55 (m, 1H), 9.03 (s, 1H), 8.53 (d, J = |
| | | 1.6 Hz, 1H), 8.25-8.24 (m, 1H), 8.13 (d, J = 9.2 Hz, 1H), 7.98 (d, J = 2.4 Hz, 1H), |
| | | 7.90 (d, J = 9.2 Hz, 1H), 7.73 (d, J = 8.0 Hz, 1H), 7.46 (s, 1H), 7.26 (d, J = 2.4 Hz, |
| | | 1H), 4.98 (s, 2H), 4.72 (d, J = 5.6 Hz, 2H), 4.24-4.21 (m, 2H), 4.15-4.05 (m, 2H), |
| | | 3.74-3.63 (m, 2H), 2.79-2.76 (m, 2H), 1.97-1.87 (m, 3H), 0.98-0.92 (m, 2H), |
| | | 0.73-0.68 (m, 2H) ppm |
| 395 | 548.2 | 1H NMR (400 MHz, DMSO-d6) δ = 9.65-9.62 (m, 1H), 9.03 (s, 1H), 8.40 (d, J = |
| | | 1.2 Hz, 1H), 8.35-8.30 (m, 1H), 8.20-8.11 (m, 2H), 7.98 (d, J = 1.6 Hz, 1H), 7.91 |
| | | (d, J = 9.2 Hz, 1H), 7.52-7.42 (m, 2H), 5.02 (s, 2H), 4.72 (d, J = 5.6 Hz, 2H), 4.27- |
| | | 4.19 (m, 2H), 4.17-4.09 (m, 2H), 3.80-3.72 (m, 2H), 2.80-2.77 (m, 2H), 2.25 (s, |
| | | 3H), 1.97-1.91 (m, 2H) ppm |
| 396 | 544.2 | 1H NMR (400 MHz, DMSO-d6) δ = 9.57-9.54 (m, 1H), 9.03 (s, 1H), 8.52 (d, J = |
| | | 1.6 Hz, 1H), 8.26 (d, J = 1.6 Hz, 1H), 8.14 (d, J = 9.2 Hz, 1H), 7.98 (d, J = 1.6 Hz, |
| | | 1H), 7.91 (d, J = 9.2 Hz, 1H), 7.73 (d, J = 7.6 Hz, 1H), 7.55-7.39 (m, 2H), 4.96 (s, |
| | | 2H), 4.72 (d, J = 5.6 Hz, 2H), 4.30-4.27 (m, 1H), 4.17-4.09 (m, 2H), 4.04-4.02 |
| | | (m, 1H), 3.70-3.60 (m, 1H), 2.80-2.79 (m, 2H), 2.24 (s, 3H), 1.97-1.92 (m, 2H), |
| | | 1.16 (d, J = 7.2 Hz, 3H) ppm |
| 397 | 564.2 | 1H NMR (400 MHz, DMSO-d6) δ = 9.64-9.62 (m, 1H), 9.48 (s, 1H), 8.78 (d, J = |
| | | 8.8 Hz, 1H), 8.63 (d, J = 5.0 Hz, 1H), 8.53 (d, J = 1.6 Hz, 1H), 8.40 (d, J = 8.8 Hz, |
| | | 1H), 8.26-8.24 (m, 1H), 8.02-7.91 (m, 2H), 7.74 (d, J = 7.8 Hz, 1H), |
| | | 7.67-7.32 (m, 2H), 4.98 (s, 2H), 4.86 (br d, J = 6.0 Hz, 2H), 4.27-4.20 (m, 2H), |
| | | 3.98 (s, 3H), 3.71-3.65 (m, 2H) ppm |
| 398 | 600.3 | 1H NMR (400 MHz, DMSO-d6) δ = 9.65-9.63 (m, 1H), 9.38 (s, 1H), 8.68 (d, J = |
| | | 8.8 Hz, 1H), 8.59-8.52 (m, 2H), 8.28-8.26 (m, 1H), 7.92 (d, J = 7.6 Hz, 1H), 7.81 |
| | | (s, 1H), 7.78-7.70 (m, 2H), 7.16 (d, J = 8.4 Hz, 1H), 4.98 (s, 2H), 4.82 (d, J = 6.0 |
| | | Hz, 2H), 4.62-4.49 (m, 1H), 4.28-4.19 (m, 2H), 3.99-3.97 (m, 2H), 3.73-3.65 |
| | | (m, 2H), 3.54-3.48 (m, 2H), 2.27-2.10 (m, 3H), 1.81 (d, J = 10.8 Hz, 2H), 1.03- |
| | | 0.94 (m, 2H), 0.70-0.58 (m, 2H) ppm |
| 399 | 541.2 | 1H NMR (400 MHz, DMSO-d6) δ = 9.67-9.64 (m, 1H), 9.45 (s, 1H), 8.81-8.73 (m, |
| | | 1H), 8.72-8.66 (m, 1H), 8.54 (d, J = 2.0 Hz, 1H), 8.33 (d, J = 8.0 Hz, 1H), 8.28- |
| | | 8.26 (m, 1H), 8.14 (d, J = 8.0 Hz, 1H), 7.86 (s, 1H), 7.74 (d, J = 8.0 Hz, 1H), 7.45- |
| | | 6.85 (m, 1H), 4.99 (s, 2H), 4.84 (d, J = 5.2 Hz, 2H), 4.30-4.19 (m, 2H), 4.14 (s, |
| | | 3H), 3.75-3.63 (m, 2H) ppm |
| 400 | 563.3 | 1H NMR (400 MHz, METHANOL-d4) δ = 8.88 (br s, 1H), 8.61 (d, J = 2.0 Hz, 1H), |
| | | 8.31 (s, 1H), 8.24-8.13 (m, 2H), 7.63 (d, J = 8.0 Hz, 1H), 7.54 (s, 1H), 7.43 (d, J = |
| | | 9.4 Hz, 1H), 5.04 (s, 2H), 4.81 (s, 2H), 4.38-4.31 (m, 2H), 4.22-4.20 (m, 2H), 4.04- |
| | | 3.96 (m, 2H), 3.72-3.70 (m, 2H), 3.57-3.49 (m, 2H), 3.29 (s, 3H), 2.88-2.86 (m, |
| | | 2H), 2.17-2.08 (m, 2H) ppm |
| 401 | 562.3 | 1H NMR (400 MHz, DMSO-d6) δ = 9.64-9.62 (m, 1H), 9.46 (s, 1H), 8.69 (d, J = |
| | | 8.8 Hz, 1H), 8.51 (d, J = 1.6 Hz, 1H), 8.24-8.22 (m, 1H), 7.96 (d, J = 8.8 Hz, 1H), |
| | | 7.78 (s, 1H), 7.72 (d, J = 7.8 Hz, 1H), 7.62-7.60 (m, 1H), 6.85-6.83 (m, 1H), 6.36- |
| | | 6.34 (m, 1H), 4.97 (s, 2H), 4.82 (d, J = 5.6 Hz, 2H), 4.25-4.19 (m, 2H), 3.74- |
| | | 3.65 (m, 6H), 3.12-3.08 (m, 4H) ppm |
| 402 | 593.3 | 1H NMR (400 MHz, DMSO-d6) δ = 9.61-9.59 (m, 1H), 9.35 (s, 1H), 8.57-8.49 (m, |
| | | 2H), 8.47 (s, 1H), 8.24-8.22 (m, 1H), 8.01 (d, J = 8.8 Hz, 1H), 7.77-7.67 (m, 2H), |
| | | 7.26-7.24 (m, 1H), 6.85-6.83 (m, 1H), 4.97 (s, 2H), 4.80 (d, J = 5.6 Hz, 2H), 4.20- |
| | | 4.18 (m, 4H), 3.70-3.63 (m, 2H), 3.58-3.56 (m, 2H), 3.37 (br s, 2H), 3.27 (s, 5H) ppm |
| 404 | 559.2 | 1H NMR (400 MHz, DMSO-d6) δ = 9.719.68 (m, 1H), 9.43 (s, 1H), 8.65 (d, J = 8.6 |
| | | Hz, 1H), 8.48-8.38 (m, 2H), 8.24-8.13 (m, 2H), 7.84 (s, 1H), 7.48 (d, J = 7.8 Hz, |
| | | 1H), 7.14-6.83 (m, 1H), 5.02 (s, 2H), 4.82 (br d, J = 5.6 Hz, 2H), 4.27-4.20 (m, |
| | | 2H), 4.02 (s, 3H), 3.79-3.73 (m, 2H) ppm |
| 405 | 555.2 | 1H NMR (400 MHz, DMSO-d6) δ = 9.64-9.62 (m, 1H), 9.43 (s, 1H), 8.66 (d, J = |
| | | 8.6 Hz, 1H), 8.52 (d, J = 1.8 Hz, 1H), 8.48-8.43 (m, 1H), 8.27-8.25 (m, 1H), 8.21 |
| | | (d, J = 8.8 Hz, 1H), 7.84 (s, 1H), 7.74 (d, J = 7.8 Hz, 1H), 7.48 (d, J = |
| | | 7.8 Hz, 1H), 7.17-6.80 (m, 1H), 4.96 (s, 2H), 4.87-4.77 (m, 2H), 4.35-4.20 (m, |
| | | 1H), 4.06-3.96 (m, 4H), 3.70-3.57 (m, 1H), 1.20-1.13 (m, 3H) ppm |
| 40€ | 567.3 | 1H NMR (400 MHz, DMSO-d6) δ 9.82 (t, J = 5.8 Hz, 1H), 8.98 (dd, J = 9.0, 1.0 Hz, |
| | | 1H), 8.89 (d, J = 9.0 Hz, 1H), 8.61 (d, J = 2.2 Hz, 1H), 8.30 (dd, J = 8.4, 2.2 Hz, |
| | | 1H), 8.19 (d, J = 1.0 Hz, 1H), 7.96-7.91 (m, 2H), 7.79 (dd, J = 8.5, 7.4 Hz, 1H), |
| | | 7.09 (d, J = 8.5 Hz, 1H), 5.13 (d, J = 5.7 Hz, 2H), 4.33 (dd, 2H), 3.74-3.62 (m, 2H), |
| | | 3.42 (s, 3H), 2.55 (m, 2H), 1.22 (d, J = 6.2 Hz, 6H). |
| 407 | 609.3 | 1H NMR (400 MHz, DMSO-d6) δ = 9.93-9.90 (m, 1H), 9.42-9.40 (m, 2H), 8.97 (d, |
| | | J = 1.6 Hz, 1H), 8.68-8.61 (m, 2H), 8.44 (s, 1H), 7.91-7.88 (m, 2H), 7.77-7.73 (m, |
| | | 1H), 7.04 (d, J = 8.4 Hz, 1H), 4.86 (br d, J = 6.0 Hz, 2H), 4.32 (br d, J = 12.4 Hz, |
| | | 2H), 3.74-3.72 (m, 2H), 3.70-3.66 (m, 2H), 2.54-2.52 (m, 4H), 2.30 (br d, J = 3.6 |
| | | Hz, 2H), 1.22 (d, J = 6.42 Hz, 6H) ppm |
| 408 | 530.3 | 1H NMR (400 MHz, DMSO-d6) δ = 9.62-9.47 (m, 1H), 8.91 (s, 1H), 8.52 (d, J = |
| | | 2.0 Hz, 1H), 8.41 (br s, 1H), 8.31-8.22 (m, 1H), 8.18 (d, J = 9.2 Hz, 1H), 7.73 (d, |
| | | J = 8.0 Hz, 1H), 7.49 (d, J = 7.6 Hz, 1H), 7.40 (d, J = 9.2 Hz, 1H), 7.30 (s, 1H), 7.08 |
| | | (d, J = 7.6 Hz, 1H), 4.97 (s, 2H), 4.89 (s, 2H), 4.67 (d, J = 5.6 Hz, 2H), 4.31-4.17 |
| | | (m, 2H), 4.10-3.99 (m, 2H), 3.74-3.64 (m, 2H), 2.92-2.83 (m, 2H), 2.43 (s, 3H) ppm |
| 409 | 588.2 | 1H NMR (400 MHz, DMSO-d6) δ = 9.63-9.60 (m, 1H), 9.37 (s, 1H), 8.58-8.48 (m, |
| | | 2H), 8.26-8.24 (m, 1H), 8.06 (d, J = 8.8 Hz, 1H), 7.77 (s, 1H), 7.74-7.70 (m, 2H), |
| | | 7.45 (d, J = 7.6 Hz, 1H), 7.06-7.02 (m, 1H), 4.97 (s, 2H), 4.81 (d, J = 6.0 Hz, 2H), |
| | | 4.64-4.53 (m, 1H), 4.37-4.18 (m, 4H), 3.98-3.83 (m, 1H), 3.71-3.63 (m, 2H), |
| | | 3.53-3.40 (m, 1H), 3.22-3.15 (m, 1H), 2.81-2.72 (m, 1H), 2.64-2.58 (m, 1H), |
| | | 2.18-2.01 (m, 2H) ppm |
| 410 | 588.2 | 1H NMR (400 MHz, DMSO-d6) δ = 9.63-9.61 (m, 1H), 9.37 (s, 1H), 8.58-8.47 (m, |
| | | 2H), 8.26-8.24 (m, 1H), 8.07 (d, J = 8.8 Hz, 1H), 7.76 (s, 1H), 7.74-7.70 (m, 2H), |
| | | 7.45 (d, J = 7.6 Hz, 1H), 7.06-7.02 (m, 1H), 4.97 (s, 2H), 4.81 (d, J = 6.0 Hz, 2H), |
| | | 4.64-4.53 (m, 1H), 4.37-4.17 (m, 4H), 3.98-3.83 (m, 1H), 3.71-3.63 (m, 2H), |
| | | 3.53-3.40 (m, 1H), 3.22-3.14 (m, 1H), 2.81-2.72 (m, 1H), 2.64-2.58 (m, 1H), |
| | | 2.18-2.01 (m, 2H) ppm |
| 411 | 519.2 | 1H NMR (400 MHz, DMSO-d6) δ = 9.63-9.60 (m, 1H), 9.37 (s, 1H), 8.58 (d, J = |
| | | 8.4 Hz, 1H), 8.52 (d, J = 2.0 Hz, 1H), 8.30-8.23 (m, 2H), 8.19 (d, J = 8.4 Hz, 1H), |
| | | 7.79 (s, 1H), 7.73 (d, J = 7.6 Hz, 1H), 7.04 (d, J = 7.6 Hz, 1H), 4.96 (s, 2H), 4.81 (d, |
| | | J = 6.0 Hz, 2H), 4.30-4.26 (m, 1H), 4.05-3.95 (m, 4H), 3.68-3.61 (m, 1H), 2.49 |
| | | (s, 3H), 1.16 (d, J = 7.2 Hz, 3H) ppm |
| 412 | 574.3 | 1H NMR (400 MHz, DMSO-d6) δ = 9.64-9.63 (m, 1H), 9.39 (s, 1H), 8.66 (s, 2H), |
| | | 8.54 (d, J = 1.6 Hz, 1H), 8.33 (s, 1H), 8.28-8.25 (m, 1H), 7.92 (d, J = 7.2 Hz, 1H), |
| | | 7.82 (s, 1H), 7.77-7.71 (m, 2H), 7.13 (d, J = 8.4 Hz, 1H), 4.98 (s, 2H), 4.82 (d, J = |
| | | 5.6 Hz, 2H), 4.27-4.19 (m, 2H), 3.73-3.64 (m, 2H), 3.39-3.34 (m, 1H), 3.23 (s, |
| | | 3H), 3.19-3.13 (m, 4H), 2.74-2.72 (m, 1H), 1.50-1.48 (m, 1H), 1.23-1.10 (m, |
| | | 1H), 0.65-0.62 (m, 1H) ppm |
| 413 | 574.2 | 1H NMR (400 MHz, DMSO-d6) δ = 9.64-9.63 (m, 1H), 9.39 (s, 1H), 8.65 (s, 2H), |
| | | 8.54 (d, J = 1.6 Hz, 1H), 8.33 (s, 1H), 8.28-8.26 (m, 1H), 7.92 (d, J = 7.2 Hz, 1H), |
| | | 7.82 (s, 1H), 7.77-7.71 (m, 2H), 7.12 (d, J = 8.4 Hz, 1H), 4.98 (s, 2H), 4.82 (d, J = |
| | | 5.6 Hz, 2H), 4.27-4.19 (m, 2H), 3.73-3.65 (m, 2H), 3.39-3.34 (m, 1H), 3.23 (s, |
| | | 3H), 3.19-3.13 (m, 4H), 2.74-2.73 (m, 1H), 1.50-1.48 (m, 1H), 1.23-1.10 (m, |
| | | 1H), 0.65-0.62 (m, 1H) ppm |
| 414 | 544.2 | 1H NMR (400 MHz, DMSO-d6) δ = 9.63-9.60 (m, 1H), 9.35 (s, 1H), 8.64-8.62 (m, |
| | | 2H), 8.54 (d, J = 1.6 Hz, 1H), 8.31 (br s, 1H), 8.27-8.25 (m, 1H), 7.77(s, 1H), 7.74- |
| | | 7.72 (m, 2H), 7.37 (d, J = 7.6 Hz, 1H), 4.98 (s, 2H), 4.81 (d, J = 5.6 Hz, 2H), 4.24- |
| | | 4.21 (m, 2H), 3.69-3.64 (m, 3H), 3.19(s, 3H), 2.84-2.74 (m, 2H), 1.87-1.78 (m, 2H), |
| | | 1.19 (d, J = 6.4 Hz, 3H) ppm |
| 415 | 587.2 | 1H NMR (400 MHz, DMSO-d6) δ = 9.39 (s, 1H), 9.32 (m, 1H), 8.70-8.60 (m, 2H), |
| | | 8.47 (d, J = 2.0 Hz, 1H), 8.10 (m, 1H), 7.91 (d, J = 7.4 Hz, 1H), 7.78-7.71 (m, 2H), |
| | | 7.18 (d, J = 8.8 Hz, 1H), 7.03 (d, J = 8.4 Hz, 1H), 4.78 (br d, J = 5.6 Hz, 2H), 4.32 |
| | | (br d, J = 11.6 Hz, 2H), 3.73-3.63 (m, 2H), 3.52-3.43 (m, 4H), 3.08 (s, 3H), 2.55- |
| | | 2.53 (m, 2H), 2.17-2.07 (m, 2H), 1.22 (d, J = 6.1 Hz, 6H) ppm |
| 416 | 588.2 | 1H NMR (400 MHz, DMSO-d6) δ = 9.59-9.52 (m, 1H), 9.39 (s, 1H), 8.71-8.58 (m, |
| | | 2H), 8.46 (d, J = 2.4 Hz, 1H), 8.42 (s, 1H), 8.27-8.24 (m, 1H), 7.90 (d, J = 7.2 Hz, |
| | | 1H), 7.70 (s, 2H), 7.38 (d, J = 8.4 Hz, 1H), 7.03 (d, J = 8.8 Hz, 1H), 4.80 (d, J = 5.6 |
| | | Hz, 2H), 4.31 (d, J = 11.2 Hz, 2H), 4.25-4.14 (m, 1H), 3.69-3.65 (m, 2H), 3.64- |
| | | 3.58 (m, 2H), 2.32-2.05 (m, 4H), 1.43 (d, J = 6.4 Hz, 3H), 1.21 (d, J = 6.4 Hz, 6H) ppm |
| 417 | 533.2 | 1H NMR (400 MHz, DMSO-d6) δ = 9.64-9.61 (m, 1H), 9.41 (s, 1H), 8.73-8.58 (m, |
| | | 2H), 8.54 (s, 1H), 8.33-8.24 (m, 1H), 8.06 (s, 1H), 7.84 (s, 1H), 7.75 (d, J = 8.0 Hz, |
| | | 1H), 6.80 (s, 1H), 5.02-4.93 (m, 2H), 4.87-4.75 (m, 2H), 4.49-4.47 (m, 2H), 4.35- |
| | | 4.24 (m, 1H), 4.06-3.95 (m, 1H), 3.71-3.61 (m, 1H), 2.39 (s, 3H), 1.39-1.37 (m, |
| | | 3H), 1.17 (d, J = 7.0 Hz, 3H) ppm |
| 418 | 588.3 | 1H NMR (400 MHz, DMSO-d6) δ = 9.63-9.51 (m, 1H), 9.39 (s, 1H), 8.72-8.58 (m, |
| | | 2H), 8.46 (d, J = 2.4 Hz, 1H), 8.33-8.19 (m, 1H), 7.90 (d, J = 7.2 Hz, 1H), 7.81- |
| | | 7.67 (m, 2H), 7.38 (d, J = 8.4 Hz, 1H), 7.03 (d, J = 8.8 Hz, 1H), 4.80 (d, J = 6.0 Hz, |
| | | 2H), 4.31 (brd, J = 11.2 Hz, 2H), 4.23-4.13 (m, 1H), 3.71-3.59 (m, 4H), 2.34- |
| | | 2.11 (m, 4H), 1.43 (d, J = 6.0 Hz, 3H), 1.21 (d, J = 6.4 Hz, 6H) ppm |
| 419 | 554.2 | 1H NMR (400 MHz, DMSO-d6) δ = 9.73-9.53 (m, 1H), 9.39 (s, 1H), 8.62-8.46 (m, |
| | | 2H), 8.36-8.20 (m, 1H), 8.07 (d, J = 8.4 Hz, 1H), 7.87 (d, J = 8.0 Hz, 1H), 7.80 (s, |
| | | 1H), 7.73 (d, J = 7.6 Hz, 1H), 7.39-7.16 (m, 2H), 4.97 (s, 2H), 4.82 (d, J = 6.0 Hz, |
| | | 2H), 4.34-4.12 (m, 2H), 3.92 (s, 3H), 3.76-3.58 (m, 2H), 2.08 (s, 3H) ppm |
| 420 | 531.2 | 1H NMR (400 MHz, DMSO-d6) δ = 9.56-9.54 (m, 1H), 9.02 (s, 1H), 8.52 (d, J = |
| | | 1.6 Hz, 1H), 8.45 (br s, 1H), 8.28-8.21 (m, 2H), 7.73 (d, J = 7.8 Hz, 1H), 7.52 (d, |
| | | J = 9.2 Hz, 1H), 7.40 (s, 1H), 7.35 (d, J = 8.2 Hz, 1H), 6.79 (s, 1H), 6.76-6.70 (m, |
| | | 1H), 4.98 (s, 2H), 4.71 (d, J = 6.0 Hz, 2H), 4.28-4.20 (m, 6H), 3.71-3.66 (m, 2H), |
| | | 2.26 (s, 3H) ppm |
| 421 | 557.3 | 1H NMR (400 MHz, DMSO-d6) δ 9.63 (t, J = 5.9 Hz, 1H), 9.38 (s, 1H), 8.62 (d, J = |
| | | 8.6 Hz, 1H), 8.51 (d, J = 1.8 Hz, 1H), 8.24 (dd, J = 7.8, 1.9 Hz, 1H), 8.01 (d, J = 8.6 |
| | | Hz, 1H), 7.79 (s, 1H), 7.73 (d, J = 7.8 Hz, 1H), 7.26-7.14 (m, 2H), 6.70 (dd, J = |
| | | 7.4, 1.3 Hz, 1H), 4.97 (s, 2H), 4.81 (d, J = 5.8 Hz, 2H), 4.26-4.15 (m, 2H), 3.78 |
| | | (dt, J = 11.3, 3.2 Hz, 2H), 3.72-3.62 (m, 2H), 3.62-3.55 (m, 2H), 3.45 (td, J = |
| | | 11.5, 2.7 Hz, 1H), 3.30 (d, J = 7.0 Hz, 2H), 3.04 (td, J = 12.3, 3.5 Hz, 1H), 2.84 (dd, |
| | | J = 16.4, 7.4 Hz, 1H). |
| 422 | 520.25 | 1H NMR (400 MHz, DMSO-d6) δ 9.85 (t, J = 5.8 Hz, 1H), 9.00 (d, J = 9.0 Hz, 1H), |
| | | 8.88 (d, J = 9.0 Hz, 1H), 8.42-8.34 (m, 2H), 8.24 (s, 1H), 8.17 (dd, J = 10.1, 1.7 Hz, |
| | | 1H), 7.90 (t, J = 7.8 Hz, 1H), 7.53 (d, J = 7.6 Hz, 1H), 5.14 (d, J = 5.6 Hz, 2H), 5.02 |
| | | (s, 2H), 4.23 (dd, J = 6.3, 3.5 Hz, 2H), 3.76 (dd, J = 5.9, 3.6 Hz, 2H), 2.31-2.21 (m, |
| | | 1H), 1.18-1.02 (m, 4H). |
| 423 | 516.25 | 1H NMR (400 MHz, DMSO-d6) δ 9.78 (t, J = 5.8 Hz, 1H), 9.00 (dd, J = 9.0, 1.0 Hz, |
| | | 1H), 8.87 (d, J = 9.0 Hz, 1H), 8.52 (d, J = 1.9 Hz, 1H), 8.37 (dd, J = 7.8, 1.0 Hz, |
| | | 1H), 8.27 (dd, J = 7.8, 1.9 Hz, 1H), 8.22 (d, J = 1.0 Hz, 1H), 7.89 (t, J = 7.8 Hz, 1H), |
| | | 7.74 (d, J = 7.9 Hz, 1H), 7.53 (dd, J = 7.9, 1.0 Hz, 1H), 5.14 (d, J = 5.7 Hz, 2H), |
| | | 4.96 (s, 2H), 4.29 (dd, J = 13.2, 2.4 Hz, 1H), 4.02 (dd, J = 13.3, 5.8 Hz, 1H), 3.73- |
| | | 3.58 (m, 1H), 2.30-2.21 (m, 1H), 1.23-0.99 (m, 7H). |
| 424 | 520.2 | 1H NMR (300 MHz, DMSO-d6) δ 9.99 (s, 1H), 9.84 (t, J = 5.8 Hz, 1H), 9.59 (s, 1H), |
| | | 8.48 (d, J = 1.7 Hz, 1H), 8.37 (dd, J = 7.8, 1.0 Hz, 1H), 8.26 (dd, J = 10.1, 1.7 Hz, |
| | | 1H), 8.07-7.93 (m, 2H), 7.62 (dd, J = 7.9, 1.0 Hz, 1H), 5.10 (s, 2H), 4.94 (d, J = |
| | | 5.7 Hz, 2H), 4.30 (dd, J = 6.4, 3.5 Hz, 2H), 3.84 (t, J = 4.8 Hz, 2H), 2.38-2.31 (m, |
| | | 1H), 1.27-1.09 (m, 4H). |
| 425 | 570.3 | 1H NMR (400 MHz, DMSO-d6) δ = 9.63-9.62 (m, 1H), 9.35 (s, 1H), 8.74 (d, J = 8.8 |
| | | Hz, 1H), 8.63 (d, J = 8.4 Hz, 1H), 8.54 (d, J = 1.6 Hz, 1H), 8.28-8.25 (m, 1H), 8.21 |
| | | (s, 1H), 7.84 (d, J = 7.6 Hz, 1H), 7.78 (s, 1H), 7.75 (d, J = 7.6 Hz, 1H), 7.41 (d, J = |
| | | 7.6 Hz, 1H), 4.98 (s, 2H), 4.81 (br d, J = 5.6 Hz, 2H), 4.24-4.22 (m, 2H), 3.76-3.67 |
| | | (m, 1H), 3.86-2.69 (m, 2H), 2.91-2.82 (m, 1H), 2.76-2.69 (m, 2H), 1.78-1.75 (m, |
| | | 2H), 1.19-1.13 (m, 6H), 0.78-0.77 (m, 1H), 0.67-0.59 (m, 2H) ppm |
| 426 | 546.3 | 1H NMR (400 MHz, DMSO-d6) δ = 9.62-9.60 (m, 1H), 9.33 (s, 1H), 8.65-8.50 (m, |
| | | 3H), 8.46 (s, 1H), 8.26-8.24 (m, 1H), 7.82 (d, J = 8.0 Hz, 1H), 7.77-7.69 (m, 2H), |
| | | 7.10 (d, J = 7.8 Hz, 1H), 4.98 (s, 2H), 4.80 (d, J = 5.6 Hz, 2H), 4.32-4.21 (m, 2H), |
| | | 4.20-4.08 (m, 2H), 3.78-3.62 (m, 3H), 3.18 (s, 3H), 1.21 (d, J = 6.4 Hz, 3H) ppm |
| 427 | 553.2 | 1H NMR (400 MHz, DMSO-d6) δ = 9.65-9.63 (m, 1H), 9.42 (s, 1H), 8.79-8.72 (m, |
| | | 1H), 8.69-8.63 (m, 1H), 8.54 (d, J = 1.6 Hz, 1H), 8.49-8.39 (m, 1H), 8.33 (d, J = |
| | | 7.4 Hz, 1H), 8.27-8.24 (m, 1H), 8.00-7.98 (m, 1H), 7.85 (s, 1H), |
| | | 7.74 (d, J = 7.8 Hz, 1H), 7.13 (d, J = 8.4 Hz, 1H), 4.99 (s, 2H), 4.94-4.86 (m, 1H), |
| | | 4.83 (br d, J = 5.6 Hz, 2H), 4.28-4.18 (m, 2H), 3.72-3.62 (m, 2H), 2.21-2.15 (m, |
| | | 1H), 1.94-1.87 (m, 1H) ppm |
| 428 | 586.5 | 1H NMR (400 MHz, CHLOROFORM-d) δ = 9.25 (s, 1H), 8.62-8.53 (m, 2H), 8.37 |
| | | (d, J = 8.8 Hz, 1H), 8.20-8.18 (m, 1H), 8.04-7.98 (m, 2H), 7.83-7.76 (m, 1H), |
| | | 7.69-7.67 (m, 1H), 7.49 (d, J = 7.8 Hz, 1H), 7.11 (d, J = 8.4 Hz, 1H), |
| | | 5.06-5.02 (m, 2H), 5.01-4.96 (m, 2H), 4.81-4.79 (m, 2H), 4.65-4.63 (m, 2H), |
| | | 4.41-4.36 (m, 2H), 4.19 (d, J = 7.2 Hz, 2H), 3.44-3.40 (m, 3H), 2.54-2.46 (m, |
| | | 1H), 0.98 (br d, J = 5.2 Hz, 2H), 0.75-0.73 (m, 2H) ppm |
| 429 | 544.2 | 1H NMR (400 MHz, DMSO-d6) δ = 9.56-9.54 (m, 1H), 9.02 (s, 1H), 8.54 (d, J = |
| | | 1.4 Hz, 1H), 8.28-8.21 (m, 1H), 8.12 (d, J = 9.4 Hz, 1H), 7.93 (s, 1H), 7.82 (d, J = |
| | | 9.2 Hz, 1H), 7.74 (d, J = 7.8 Hz, 1H), 7.45 (s, 1H), 4.98 (s, 2H), 4.73 (br d, J = 5.6 |
| | | Hz, 2H), 4.28-4.19 (m, 2H), 4.16-4.09 (m, 2H), 3.74-3.65 (m, 2H), 2.76-2.74 |
| | | (m, 2H), 2.23-2.20 (m, 3H), 2.20-2.15 (m, 3H), 1.99-1.91 (m, 2H) ppm |
| 430 | 530.3 | 1H NMR (400 MHz, DMSO-d6) δ = 9.63-9.61 (m, 1H), 9.40 (s, 1H), 8.60 (d, J = |
| | | 8.8 Hz, 1H), 8.52 (s, 1H), 8.23 (d, J = 7.6 Hz, 1H), 8.05 (d, J = 8.8 Hz, 1H), 7.80 (s, |
| | | 1H), 7.72 (d, J = 8.0 Hz, 1H), 7.53 (d, J = 7.6 Hz, 1H), 7.17-7.15 (m, 1H), 7.01 (d, |
| | | J = 6.4 Hz, 1H), 4.96 (s, 2H), 4.81 (br d, J = 5.6 Hz, 2H), 4.21-4.20 (m, 2H), 3.68- |
| | | 3.67 (m, 2H), 3.50 (s, 3H), 2.23-2.20 (m, 1H), 1.02-1.00 (m, 2H), 0.75-0.73 (m, 2H) ppm |
| 431 | 622.2 | 1H NMR (400 MHz, DMSO-d6) δ = 9.73-9.71 (m, 1H), 9.40 (s, 1H), 8.65-8.63 (m, |
| | | 2H), 8.51 (d, J = 1.6 Hz, 2H), 8.43 (d, J = 1.6 Hz, 1H), 7.92 (d, J = 7.4 Hz, 1H), 7.86 |
| | | (s, 1H), 7.78-7.72 (m, 1H), 7.03 (d, J = 8.4 Hz, 1H), 5.28-5.11 (m, 2H), 4.82 (br d, |
| | | J = 5.4 Hz, 2H), 4.34-4.28 (m, 3H), 4.02-4.00 (m, 1H), 3.80-3.72 (m, 1H), 3.71- |
| | | 3.64 (m, 2H), 2.54-2.52 (m, 2H), 1.22 (d, J = 6.2 Hz, 6H), 1.18 (d, J = 7.0 Hz, 3H) ppm |
| 432 | 622.2 | 1H NMR (400 MHz, DMSO-d6) δ = 9.73-9.70 (m, 1H), 9.40 (s, 1H), 8.65-8.64 (m, |
| | | 2H), 8.50 (d, J = 1.6 Hz, 2H), 8.43 (d, J = 1.6 Hz, 1H), 7.92 (d, J = 7.4 Hz, 1H), 7.86 |
| | | (s, 1H), 7.78-7.72 (m, 1H), 7.03 (d, J = 8.4 Hz, 1H), 5.28-5.12 (m, 2H), 4.81 (br d, |
| | | J = 5.4 Hz, 2H), 4.34-4.28 (m, 3H), 4.02-4.00 (m, 1H), 3.80-3.72 (m, 1H), 3.71- |
| | | 3.64 (m, 2H), 2.54-2.52 (m, 2H), 1.22 (d, J = 6.2 Hz, 6H), 1.18 (d, J = 7.0 Hz, 3H) ppm |
| 433 | 614.3 | 1H NMR (400 MHz, DMSO-d6) δ = 9.55-9.52 (m, 1H), 9.38 (s, 1H), 8.68-8.58 (m, |
| | | 2H), 8.44 (d, J = 2.2 Hz, 1H), 8.31 (s, 1H), 8.25-8.23 (m, 1H), 7.88 (d, J = 7.4 Hz, |
| | | 1H), 7.78-7.70 (m, 2H), 7.37 (d, J = 8.4 Hz, 1H), 7.02 (d, J = 8.4 Hz, 1H), 4.79 (br |
| | | d, J = 5.6 Hz, 2H), 4.30 (br d, J = 11.2 Hz, 2H), 3.71-3.60 (m, 3H), 3.60-3.50 (m, |
| | | 1H), 3.49-3.41 (m, 1H), 2.46 (br s, 5H), 1.22-1.19 (m, 6H), 0.65-0.47 (m, 3H), |
| | | 0.43-0.32 (m, 1H) ppm |
| 434 | 537.2 | 1H NMR (400 MHz, DMSO-d6) δ = 9.96-9.93 (m, 1H), 9.46 (d, J = 5.2 Hz, 2H), 9.43 |
| | | (d, J = 1.6 Hz, 1H), 8.97 (d, J = 1.6 Hz, 1H), 8.79 (s, 1H), 8.76 (d, J = 8.4 Hz, 1H), |
| | | 8.56 (d, J = 8.8 Hz, 1H), 8.49-8.46 (m, 1H), 7.95 (s, 1H), 4.88 (br d, J = 5.6 Hz, |
| | | 2H), 3.75-3.72 (m, 2H), 2.55-2.54 (m, 2H), 2.35-2.28 (m, 3H), 1.16-1.14 (m, 4H) ppm |
| 435 | 614.3 | 1H NMR (400 MHz, DMSO-d6) δ = 9.57-9.56 (m, 1H), 9.39 (s, 1H), 8.67-8.60 (m, |
| | | 2H), 8.45 (d, J = 2.4 Hz, 1H), 8.27-8.24 (m, 1H), 7.90 (d, J = 7.2 Hz, 1H), 7.77-7.73 |
| | | (m, 2H), 7.39 (d, J = 8.4 Hz, 1H), 7.03 (d, J = 8.8 Hz, 1H), 4.80 (br d, J = 5.6 Hz, |
| | | 2H), 4.32 (brd, J = 11.6 Hz, 2H), 3.69-3.63 (m, 3H), 3.61-3.52 (m, 1H), 3.48-3.46 |
| | | (m, 1H), 2.52 (br s, 2H), 2.46-2.29 (m, 3H), 1.21 (d, J = 6.0 Hz, 6H), 0.62-0.57 (m, |
| | | 2H), 0.50-0.37 (m, 2H) ppm |
| 436 | 588.3 | 1H NMR (400 MHz, DMSO-d6) δ = 9.63 (s, 1H), 9.39 (s, 1H), 8.71-8.59 (m, 2H), |
| | | 8.54 (d, J = 1.6 Hz, 1H), 8.36-8.34 (m, 1H), 8.27-8.25 (m, 1H), 7.87-7.66 (m, |
| | | 4H), 6.81 (d, J = 8.4 Hz, 1H), 4.98 (s, 2H), 4.82 (d, J = 5.6 Hz, 2H), 4.79-4.70 (m, |
| | | 1H), 4.26-4.20 (m, 2H), 3.71-3.65 (m, 2H), 3.37-3.36 (m, 2H), 3.27 (s, 3H), 3.05 |
| | | (s, 3H), 2.40-2.30 (m, 3H), 2.01-1.90 (m, 2H) ppm |
| 437 | 588.4 | 1H NMR (400 MHz, DMSO-d6) δ = 9.64-9.61 (m, 1H), 9.39 (s, 1H), 8.66 (d, J = |
| | | 8.8 Hz, 1H), 8.59 (d, J = 8.8 Hz, 1H), 8.54 (s, 1H), 8.26-8.25 (m, 1H), 7.83-7.80 |
| | | (m, 2H), 7.74 (d, J = 7.6 Hz, 1H), 7.68-7.66 (m, 1 H), 7.02 (d, J = 8.4 Hz, 1H), 4.98 |
| | | (s, 2H), 4.81 (d, J = 6 Hz, 2H), 4.24-4.20 (m, 2H), 3.71-3.65 (m, 5H), 3.43-3.30 |
| | | (m, 5H), 2.11-2.01 (m, 1 H), 2.00-1.98 (m, 1H), 1.89-1.86 (m, 2H), 1.64-1.60 |
| | | (m, 2 H), 0.94 (d, J = 6.8 Hz, 1H) ppm |
| 438 | 588.4 | 1H NMR (400 MHz, DMSO-d6) δ = 9.64-9.60 (m, 1H), 9.39 (s, 1H), 8.66 (d, J = |
| | | 8.8 Hz, 1H), 8.58 (d, J = 8.8 Hz, 1H), 8.54 (s, 1H), 8.26-8.25 (m, 1H), 7.83-7.80 |
| | | (m, 2H), 7.74 (d, J = 7.6 Hz, 1H), 7.68-7.65 (m, 1 H), 7.02 (d, J = 8.4 Hz, 1H), 4.98 |
| | | (s, 2H), 4.81 (d, J = 6 Hz, 2H), 4.24-4.20 (m, 2H), 3.71-3.65 (m, 5H), 3.43-3.30 |
| | | (m, 5H), 2.11-2.01 (m, 1 H), 2.00-1.98 (m, 1H), 1.89-1.86 (m, 2H), 1.64-1.61 |
| | | (m, 2 H), 0.94 (d, J = 6.8 Hz, 1H) ppm |
| 439 | 546.2 | 1H NMR (400 MHz, DMSO-d6) δ = 9.56-9.54 (m, 1H), 9.07 (s, 1H), 8.52 (d, J = |
| | | 1.6 Hz, 1H), 8.28-8.21 (m, 1H), 8.20-8.12 (m, 1H), 8.08 (d, J = 5.8 Hz, 1H), 7.91 |
| | | (d, J = 9.4 Hz, 1H), 7.73 (d, J = 8.0 Hz, 1H), 7.49 (s, 1H), 6.83 (d, J = 5.8 Hz, 1H), |
| | | 4.97 (s, 2H), 4.73 (br d, J = 5.6 Hz, 2H), 4.22-4.20 (m, 2H), 4.13-4.10 (m, 2H), |
| | | 3.92-3.83 (m, 3H), 3.71-3.63 (m, 2H), 2.71-2.62 (m, 2H), 1.97-1.87 (m, 2H) ppm |
| 440 | 531.2 | 1H NMR (400 MHz, DMSO-d6) δ = 9.60-9.57 (m, 1H), 9.09 (s, 1H), 8.52 (d, J = |
| | | 1.6 Hz, 1H), 8.27-8.20 (m, 2H), 8.02 (s, 1H), 7.92 (d, J = 9.2 Hz, 1H), 7.73 (d, J = |
| | | 7.6 Hz, 1H), 7.50 (s, 1H), 4.97 (s, 2H), 4.73 (d, J = 5.6 Hz, 2H), 4.26-4.15 (m, 4H), |
| | | 3.72-3.65 (m, 2H), 2.97-2.93 (m, 2H), 2.41 (s, 3H), 2.09-2.00 (m, 2H) ppm |
| 441 | 559.2 | 1H NMR (400 MHz, DMSO-d6) δ = 9.70-9.56 (m, 1H), 9.10 (s, 1H), 8.52 (d, J = |
| | | 2.0 Hz, 1H), 8.32-8.22 (m, 1H), 8.19-8.11 (m, 1H), 7.88 (d, J = 9.2 Hz, 1H), 7.80- |
| | | 7.69 (m, 2H), 7.56 (s, 1H), 7.13 (br s, 1H), 4.98 (s, 2H), 4.76 (br d, J = 5.6 Hz, 2H), |
| | | 4.27-4.19 (m, 2H), 4.17-4.07 (m, 2H), 3.74-3.63 (m, 3H), 2.92 (s, 6H), 2.82- |
| | | 2.74 (m, 2H), 2.00-1.87 (m, 2H) ppm |
| 442 | 531.2 | 1H NMR (400 MHz, DMSO-d6) δ = 9.64-9.51 (m, 1H), 9.04 (s, 1H), 8.53 (d, J = |
| | | 1.6 Hz, 1H), 8.30 (br s, 1H), 8.27-8.23 (m, 1H), 8.18 (d, J = 9.6 Hz, 1H), 7.97 (d, |
| | | J = 9.2 Hz, 1H), 7.73 (d, J = 7.6 Hz, 1H), 7.63-7.54 (m, 1H), 7.46 (s, 1H), 7.12-6.96 |
| | | (m, 2H), 4.98 (s, 2H), 4.72 (br d, J = 5.6 Hz, 2H), 4.32-4.16 (m, 4H), 3.72-3.65 |
| | | (m, 2H), 3.40-3.38 (m, 2H), 2.93 (s, 3H) ppm |
| 443 | 516.2 | 1H NMR (300 MHz, DMSO-d6) δ 9.92 (s, 1H), 9.67 (t, J = 5.8 Hz, 1H), 9.52 (s, 1H), |
| | | 8.53 (d, J = 1.8 Hz, 1H), 8.29 (ddd, J = 9.6, 7.8, 1.4 Hz, 2H), 7.96-7.86 (m, 2H), |
| | | 7.75 (d, J = 7.8 Hz, 1H), 7.54 (dd, J = 7.8, 1.0 Hz, 1H), 4.97 (s, 2H), 4.86 (d, J = 5.8 |
| | | Hz, 2H), 4.29 (dd, J = 13.2, 2.4 Hz, 1H), 4.02 (dd, J = 13.3, 5.9 Hz, 1H), 3.65 (t, J = |
| | | 7.5 Hz, 1H), 2.28-2.26 (m, 1H), 1.21-1.12 (m, 5H), 1.11-1.02 (m, 2H). |
| 444 | 602.3 | 1H NMR (400 MHz, DMSO-d6) δ = 9.57-9.56 (m, 1H), 9.40 (s, 1H), 8.71-8.59 (m, |
| | | 2H), 8.47 (d, J = 1.6 Hz, 1H), 8.19 (d, J = 2.0 Hz, 1H), 7.91 (d, J = 7.2 Hz, 1H), 7.80 |
| | | (s, 1H), 7.77-7.68 (m, 2H), 7.03 (d, J = 8.4 Hz, 1H), 4.81 (d, J = 5.6 Hz, 2H), 4.32 |
| | | (d, J = 12.4 Hz, 2H), 3.73-3.62 (m, 3H), 3.47-3.38 (m, 4H), 3.25 (s, 3H), 2.53- |
| | | 2.52 (m, 2H), 2.31-2.23 (m, 2H), 1.21 (d, J = 6.4 Hz, 6H) ppm |
| 445 | 560.2 | 1H NMR (400 MHz, DMSO-d6) δ = 9.60-9.57 (m, 1H), 9.06 (s, 1H), 8.53 (d, J = |
| | | 1.6 Hz, 1H), 8.25 (d, J = 7.6 Hz, 1H), 8.16 (d, J = 9.6 Hz, 1H), 7.99 (d, J = 9.2 Hz, |
| | | 1H), 7.74 (d, J = 7.6 Hz, 1H), 7.60 (d, J = 7.6 Hz, 1H), 7.48 (s, 1H), 7.05 (d, J = 7.6 |
| | | Hz, 1H), 4.98 (s, 2H), 4.73 (d, J = 5.2 Hz, 2H), 4.36 (s, 2H), 4.26-4.19 (m, 2H), |
| | | 4.17-4.10 (m, 2H), 3.73-3.65 (m, 2H), 3.34 (s, 3H), 2.82-2.79 (m, 2H), 2.02- |
| | | 1.89 (m, 2H) ppm |
| 446 | 602.3 | 1H NMR (400 MHz, DMSO-d6) δ = 9.56-9.54 (m, 1H), 9.40 (s, 1H), 8.69-8.61 (m, |
| | | 2H), 8.47 (d, J = 2.0 Hz, 1H), 8.19-8.17 (m, 1H), 7.91 (d, J = 7.4 Hz, 1H), 7.80 (s, |
| | | 1H), 7.78-7.67 (m, 2H), 7.03 (d, J = 8.4 Hz, 1H), 4.87-4.74 (m, |
| | | 2H), 4.37-4.27 (m, 2H), 3.74-3.62 (m, 3H), 3.46-3.40 (m, 4H), 3.25 (s, 3H), 2.52 |
| | | (br s, 2H), 2.30-2.23 (m, 2H), 1.21 (d, J = 6.2 Hz, 6H) ppm |
| 447 | 560.3 | 1H NMR (400 MHz, DMSO-d6) δ = 9.58-9.57 (m, 1H), 9.06 (s, 1H), 8.53 (d, J = |
| | | 1.6 Hz, 1H), 8.25 (d, J = 7.6 Hz, 1H), 8.18 (d, J = 9.2 Hz, 1H), 8.07 (d, J = 2.0 Hz, |
| | | 1H), 7.93 (d, J = 9.2 Hz, 1H), 7.73 (d, J = 8.0 Hz, 1H), 7.56 (d, J = 2.0 Hz, 1H), 7.49 |
| | | (s, 1H), 4.98 (s, 2H), 4.73 (d, J = 5.6 Hz, 2H), 4.37 (s, 2H), 4.27-4.19 (m, 2H), 4.18- |
| | | 4.09 (m, 2H), 3.73-3.63 (m, 2H), 3.30 (s, 3H), 2.85-2.81 (m, 2H), 1.99-1.93 (m, 2H) ppm |
| 448 | 572.2 | 1H NMR (400 MHz, DMSO-d6) δ = 9.65-9.62 (m, 1H), 9.46 (s, 1H), 8.74 (d, J = |
| | | 8.4 Hz, 1H), 8.53 (d, J = 2.0 Hz, 1H), 8.44 (d, J = 5.2 Hz, 1H), 8.37 (d, J = 8.8 Hz, |
| | | 1H), 8.27-8.24 (m, 1H), 7.92 (s, 1H), 7.81 (d, J = 5.2 Hz, 1H), 7.73 (d, J = 8.0 Hz, |
| | | 1H), 7.70 (d, J = 3.6 Hz, 1H), 7.19 (d, J = 3.6 Hz, 1H), 4.98 (s, 2H), 4.85 (d, J = 6.0 |
| | | Hz, 2H), 4.36 (d, J = 6.0 Hz, 2H), 4.28-4.16 (m, 2H), 3.81-3.76 (m, 1H), 3.72- |
| | | 3.64 (m, 2H), 3.21 (s, 3H), 1.07 (d, J = 6.0 Hz, 3H) ppm |
| 449 | 572.3 | 1H NMR (400 MHz, DMSO-d6) δ = 9.65-9.62 (m, 1H), 9.46 (s, 1H), 8.74 (d, J = |
| | | 8.8 Hz, 1H), 8.53 (d, J = 1.6 Hz, 1H), 8.44 (d, J = 5.2 Hz, 1H), 8.37 (d, J = 8.8 Hz, |
| | | 1H), 8.27-8.24 (m, 1H), 7.92 (s, 1H), 7.81 (d, J = 5.2 Hz, 1H), 7.74 (d, J = 7.6 Hz, |
| | | 1H), 7.70 (d, J = 3.6 Hz, 1H), 7.19 (d, J = 3.2 Hz, 1H), 4.98 (s, 2H), 4.85 (d, J = 5.6 |
| | | Hz, 2H), 4.36 (d, J = 5.6 Hz, 2H), 4.26-4.19 (m, 2H), 3.83-3.76 (m, 1H), 3.71- |
| | | 3.64 (m, 2H), 3.20 (s, 3H), 1.07 (d, J = 6.0 Hz, 3H) ppm |
| 450 | 515.2 | 1H NMR (400 MHz, DMSO-d6) δ = 9.76-9.74 (m, 1H), 9.45 (s, 2H), 9.35 (d, J = 2.4 |
| | | Hz, 1H), 8.78-8.77 (m, 2H), 8.75 (d, J = 8.4 Hz, 1H), 8.56 (d, J = 8.4 Hz, 1H), 7.93 |
| | | (s, 1H), 4.85 (d, J = 5.6 Hz, 2H), 3.46 (br d, J = 7.2 Hz, 4H), 2.36-2.31 (m, 1H), 1.94 |
| | | (br s, 2H), 1.86-1.81 (m, 2H), 1.26 (br s, 2H), 1.16 (s, 2H), 1.14 (d, J = 2.8 Hz, 2H) ppm |
| 451 | 587.3 | 1H NMR (400 MHz, DMSO-d6) δ = 9.75-9.72 (m, 1H), 9.40 (s, 1H), 9.35 (d, J = 2.0 |
| | | Hz, 1H), 8.78 (d, J = 2.0 Hz, 1H), 8.68-8.61 (m, 2H), 7.91 (d, J = 7.6 Hz, 1H), 7.87 |
| | | (s, 1H), 7.75 (t, J = 8.0 Hz, 1H), 7.04 (d, J = 8.4 Hz, 1H), 4.84 (d, J = 5.6 Hz, 2H), |
| | | 4.32 (br d, J = 12.0 Hz, 2H), 3.69-3.65 (m, 2H), 3.47-3.44 (m, 4H), 2.52 (br d, J = |
| | | 2.4 Hz, 2H), 1.94(br s, 2H), 1.85-1.81 (m, 2H), 1.25 (br s, 2H), 1.22 (d, J = 6.4 Hz, 6H) ppm |
| 452 | 516.2 | 1H NMR (400 MHz, METHANOL-d4) δ = 9.32 (s, 1H), 9.26 (d, J = 2.4 Hz, 1H), 8.88 |
| | | (d, J = 2.0 Hz, 1H), 8.71-8.66 (m, 1H), 8.63-8.57 (m, 1H), 8.50 (s, 1H), 8.36- |
| | | 8.34 (m, 1H), 8.01 (s, 1H), 7.80-7.78 (m, 1H), 7.41-7.35 (m, 1H), 5.13 (d, J = 1.6 |
| | | Hz, 2H), 4.95 (s, 2H), 4.46-4.42 (m, 1H), 4.18-4.14 (m, 1H), 3.63-3.54 (m, 1H), |
| | | 2.25-2.15 (m, 1H), 1.32 (d, J = 7.2 Hz, 3H), 1.18-1.14 (m, 2H), 1.09-1.03 (m, 2H) ppm |
| 453 | 537.1 | 1H NMR (400 MHz, METHANOL-d4) δ = 9.32 (s, 1H), 8.74-8.64 (m, 1H), 8.64- |
| | | 8.56 (m, 1H), 8.47 (s, 1H), 8.21 (d, J = 7.2 Hz, 1H), 8.07-8.04 (m, 1H), 7.97 (s, |
| | | 1H), 7.84-7.82 (m, 1H), 6.90 (d, J = 8.0 Hz, 1H), 5.24-5.14 (m, 1H), 5.12-5.04 |
| | | (m, 1H), 4.93 (s, 2H), 4.59-4.51 (m, 2H), 4.38-4.36(m, 1H), 4.12-4.07 (m, 1H), |
| | | 3.57-3.46 (m, 1H), 1.46-145 (m, 3H), 1.29 (d, J = 7.2 Hz, 3H) ppm |
| 454 | 614.2 | 1H NMR (400 MHz, DMSO-d6) δ = 9.65-9.62 (m, 1H), 9.40 (s, 1H), 8.72-8.60 (m, |
| | | 2H), 8.57 (d, J = 2.0 Hz, 1H), 8.33-8.30 (m, 1H), 8.07 (d, J = 8.0 Hz, 1H), 7.91 (d, |
| | | J = 7.2 Hz, 1H), 7.80 (s, 1H), 7.75-7.71 (m, 1H), 7.03 (d, J = 8.4 Hz, 1H), 4.82 (d, |
| | | J = 5.6 Hz, 2H), 4.49 (d, J = 8.0 Hz, 1H), 4.39-4.26 (m, 3H), 4.21-4.14 (m, 1H), |
| | | 3.74-3.58 (m, 4H), 2.54-2.52 (m, 2H), 1.59-1.45 (m, 1H), 1.21 (d, J = 6.4 Hz, |
| | | 6H), 0.82-0.69 (m, 2H), 0.61-0.59 (m, 1H), 0.39-0.22 (m, 1H) ppm |
| 455 | 516.2 | 1H NMR (400 MHz, METHANOL-d4) δ = 9.32 (s, 1H), 9.26 (d, J = 2.0 Hz, 1H), 8.88 |
| | | (d, J = 2.0 Hz, 1H), 8.72-8.65 (m, 1H), 8.63-8.56 (m, 1H), 8.50 (s, 1H), 8.35 (d, |
| | | J = 7.6 Hz, 1H), 8.00 (s, 1H), 7.84-7.76 (m, 1H), 7.38 (d, J = 7.2 Hz, |
| | | 1H), 5.13 (d, J = 1.6 Hz, 2H), 4.95 (s, 2H), 4.46-4.42 (m, 1H), 4.18-4.14 (m, 1H), |
| | | 3.64-3.53 (m, 1H), 2.26-2.14 (m, 1H), 1.32 (d, J = 7.2 Hz, 3H), 1.20-1.12 (m, |
| | | 2H), 1.12-1.01 (m, 2H) ppm |
| 456 | 602.2 | 1H NMR (400 MHz, DMSO-d6) δ = 9.49-9.46 (m, 1H), 9.39 (s, 1H), 8.70-8.58 (m, |
| | | 2H), 8.47-8.41 (m, 1H), 8.30 (d, J = 2.4 Hz, 1H), 8.21 (d, J = 1.6 Hz, 1H), 7.90 (d, |
| | | J = 7.4 Hz, 1H), 7.80-7.69 (m, 2H), 7.03 (d, J = 8.6 Hz, 1H), 4.79 (br d, J = 6.0 Hz, |
| | | 2H), 4.46-4.37 (m, 1H), 4.31 (br d, J = 11.4 Hz, 2H), 4.16-4.05 (m, 1H), 3.71- |
| | | 3.64 (m, 2H), 3.62-3.56 (m, 1H), 2.65-2.56 (m, 2H), 2.33 (s, 4H), 2.24-2.13 (m, |
| | | 1H), 1.33 (d, J = 7.0 Hz, 3H), 1.21 (d, J = 6.2 Hz, 6H) ppm |
| 457 | 602.2 | 1H NMR (400 MHz, DMSO-d6) δ = 9.57-9.55 (m, 1H), 9.40 (s, 1H), 8.69-8.60 (m, |
| | | 2H), 8.39 (d, J = 1.6 Hz, 1H), 8.17 (d, J = 1.4 Hz, 1H), 7.91 (d, J = 7.4 Hz, 1H), 7.82 |
| | | (s, 1H), 7.78-7.69 (m, 1H), 7.03 (d, J = 8.6 Hz, 1H), 5.10-4.95 (m, 2H), 4.81 (br d, |
| | | J = 5.0 Hz, 2H), 4.35-4.24 (m, 3H), 3.99-3.97 (m, 1H), 3.71-3.59 (m, 3H), 2.53- |
| | | 2.52 (m, 2H), 2.48-2.46 (m, 3H), 1.21 (d, J = 6.2 Hz, 6H), 1.16 (d, J = 7.2 Hz, 3H) ppm |
| 458 | 533.2 | 1H NMR (400 MHz, DMSO-d6) δ = 9.71-9.69 (m, 1H), 9.40 (s, 1H), 8.72-8.65 (m, |
| | | 1H), 8.65-8.57 (m, 1H), 8.39 (d, J = 1.2 Hz, 1H), 8.37-8.30 (m, 1H), 8.19 (d, J = |
| | | 1.6 Hz, 1H), 7.90-7.80 (m, 2H), 7.46 (d, J = 7.6 Hz, 1H), 5.13-5.02 (m, 1H), 5.00- |
| | | 4.91 (m, 1H), 4.82 (d, J = 5.2 Hz, 2H), 4.30-4.27 (m, 1H), 4.04-4.01 (m, 1H), 3.81- |
| | | 3.67 (m, 1H), 2.28-2.18 (m, 1H), 1.17 (d, J = 7.2 Hz, 3H), 1.13-1.07 (m, 2H), |
| | | 1.07-1.00 (m, 2H) ppm |
| 459 | 614.3 | 1H NMR (400 MHz, DMSO-d6) δ = 9.70-9.58 (m, 1H), 9.40 (s, 1H), 8.72-8.60 (m, |
| | | 2H), 8.56 (d, J = 2.0 Hz, 1H), 8.41 (s, 1H), 8.34-8.27 (m, 1H), 8.07 (d, J = 8.0 Hz, |
| | | 1H), 7.91 (d, J = 7.6 Hz, 1H), 7.81 (s, 1H), 7.77-7.69 (m, 1H), 7.03 (d, J = 8.4 Hz, |
| | | 1H), 4.82 (d, J = 5.6 Hz, 2H), 4.49 (d, J = 8.0 Hz, 1H), 4.37-4.27 (m, 3H), 4.23- |
| | | 4.11 (m, 1H), 3.72-3.60 (m, 4H), 2.52 (br d, J = 2.0 Hz, 2H), 1.58-1.42 (m, 1H), |
| | | 1.21 (d, J = 6.4 Hz, 6H), 0.74 (br d, J = 8.0 Hz, 2H), 0.66-0.56 (m, 1H), 0.38-0.23 |
| | | (m, 1H) ppm |
| 460 | 568.2 | 1H NMR (400 MHz, DMSO-d6) δ = 9.71-9.60 (m, 1H), 9.39 (s, 1H), 8.74-8.60 (m, |
| | | 2H), 8.54 (s, 1H), 8.41 (br s, 1H), 8.32-8.23 (m, 1H), 7.85 (d, J = 7.2 Hz, 1H), 7.81 |
| | | (s, 1H), 7.75-7.68 (m, 2H), 6.82 (d, J = 8.4 Hz, 1H), 6.40-6.02 (m, 1H), 4.98 (s, |
| | | 2H), 4.82 (br d, J = 5.6 Hz, 2H), 4.25-4.21 (m, 2H), 3.92-3.80 (m, 2H), 3.73-3.64 |
| | | (m, 2H), 3.11 (s, 3H), 2.29-2.12 (m, 2H) ppm |
| 461 | 602.3 | 1H NMR (400 MHz, DMSO-d6) δ = 9.49-9.47 (m, 1H), 9.39 (s, 1H), 8.76-8.59 (m, |
| | | 2H), 8.30 (d, J = 2.2 Hz, 1H), 8.21 (d, J = 1.8 Hz, 1H), 8.16 (s, 1H), 7.90 (d, J = 7.4 |
| | | Hz, 1H), 7.81-7.69 (m, 2H), 7.03 (d, J = 8.4 Hz, 1H), 4.79 (br d, J = 5.6 Hz, 2H), |
| | | 4.48-4.37 (m, 1H), 4.31 (br d, J = 11.2 Hz, 2H), 4.18-4.03 (m, 1H), 3.73-3.64 |
| | | (m, 2H), 3.63-3.55 (m, 1H), 2.54 (br s, 2H), 2.33 (s, 3H), 2.29-2.13 (m, 2H), 1.33 |
| | | (d, J = 7.0 Hz, 3H), 1.21 (d, J = 6.2 Hz, 6H) ppm |
| 462 | 602.3 | 1H NMR (400 MHz, DMSO-d6) δ = 9.57-9.55 (m, 1H), 9.40 (s, 1H), 8.76-8.56 (m, |
| | | 2H), 8.39 (d, J = 1.5 Hz, 1H), 8.33 (s, 1H), 8.17 (d, J = 1.2 Hz, 1H), 7.91 (d, J = 7.4 |
| | | Hz, 1H), 7.82 (s, 1H), 7.74-7.72 (m, 1H), 7.03 (d, J = 8.6 Hz, 1H), 5.04-5.02 (m, |
| | | 2H), 4.81 (brd, J = 5.6 Hz, 2H), 4.44-4.21 (m, 3H), 3.99-3.97 (m, 1H), 3.72-3.56 |
| | | (m, 3H), 2.54 (br s, 2H), 2.47 (br s, 3H), 1.21 (d, J = 6.2 Hz, 6H), 1.16 (d, J = 7.0 |
| | | Hz, 3H) ppm |
| 463 | 544.2 | 1H NMR (400 MHz, DMSO-d6) δ = 9.63-9.50 (m, 1H), 9.05-8.97 (m, 1H), 8.56- |
| | | 8.49 (m, 1H), 8.36-8.30 (m, 1H), 8.26-8.24 (m, 1H), 8.14 (d, J = 9.4 Hz, 1H), 8.00 |
| | | (d, J = 9.4 Hz, 1H), 7.74 (d, J = 8.0 Hz, 1H), 7.45 (s, 1H), 7.35 (s, 1H), 4.98 (s, 2H), |
| | | 4.73 (br d, J = 5.6 Hz, 2H), 4.26-4.21 (m, 2H), 4.14-4.10 (m, 2H), 3.71-3.68 (m, |
| | | 2H), 2.74 (s, 2H), 2.32 (s, 3H), 2.21 (s, 3H), 1.98-1.88 (m, 2H) ppm |
| 464 | 552.1 | 1H NMR (400 MHz, DMSO-d6) δ = 9.04 (s, 1H), 8.93 (d, J = 5.2 Hz, 1H), 8.77- |
| | | 8.72 (m, 2H), 8.62 (s, 1H), 8.50 (d, J = 5.2 Hz, 1H), 8.23 (d, J = 8.0 Hz, 1H), 8.02 |
| | | (s, 1H), 7.65 (d, J = 7.6 Hz, 1H), 5.07-5.02 (m, 2H), 4.95 (s, 2H), 4.39 (d, J = 13.2 |
| | | Hz, 1H), 4.12-4.07 (m, 1H), 3.48-3.46 (m, 1H), 3.27-3.26 (m, 1H), 2.59-2.54 (m, |
| | | 1 H), 2.07-2.03 (m, 1H), 1.28 (d, J = 7.2 Hz, 1H) ppm |
| 465 | 552.1 | 1H NMR (400 MHz, DMSO-d6) δ = 9.04 (s, 1H), 8.93 (d, J = 5.2 Hz, 1H), 8.77- |
| | | 8.72 (m, 2H), 8.62 (s, 1H), 8.50 (d, J = 5.2 Hz, 1H), 8.23 (d, J = 8.0 Hz, 1H), 8.02 |
| | | (s, 1H), 7.65 (d, J = 7.6 Hz, 1H), 5.07-5.01 (m, 2H), 4.95 (s, 2H), 4.38 (d, J = 13.2 |
| | | Hz, 1H), 4.12-4.07 (m, 1H), 3.48-3.47 (m, 1H), 3.27-3.25 (m, 1H), 2.59-2.54 (m, |
| | | 1 H), 2.07-2.03 (m, 1H), 1.27 (d, J = 7.2 Hz, 1H) ppm |
| 466 | 558.25 | 1H NMR (400 MHz, DMSO-d6) δ 9.63 (t, J = 5.9 Hz, 1H), 9.36 (s, 1H), 8.62 (d, J = |
| | | 1.4 Hz, 2H), 8.54 (d, J = 1.8 Hz, 1H), 8.27 (dd, J = 7.7, 1.8 Hz, 1H), 7.87-7.64 (m, |
| | | 3H), 7.47 (d, J = 7.4 Hz, 1H), 4.98 (s, 1H), 4.81 (d, J = 5.8 Hz, 1H), 4.28-4.19 (m, |
| | | 2H), 4.19-4.08 (m, 1H), 3.92 (td, J = 9.6, 9.1, 4.8 Hz, 1H), 3.82 (dd, J = 11.0, 3.6 |
| | | Hz, 2H), 3.69 (t, J = 4.8 Hz, 2H), 3.30 (s, 1H), 3.24 (d, J = 16.8 Hz, 2H), 3.09 (dd, |
| | | J = 17.3, 8.8 Hz, 1H), 2.62-2.55 (m, 1H), |
| 467 | 544.2 | 1H NMR (400 MHz, EW9897-1831-P1A, DMSO-d6) δ = 9.63 (t, J = 5.8 Hz, 1H), |
| | | 9.36 (s, 1H), 8.62 (s, 2H), 8.54 (d, J = 1.8 Hz, 1H), 8.45-8.42 (m, 1H), 8.27 (dd, |
| | | J = 1.8, 7.8 Hz, 1H), 7.77 (s, 1H), 7.74 (dd, J = 2.0, 7.7 Hz, 2H), 7.36 (d, J = 7.3 Hz, |
| | | 1H), 4.98 (s, 2H), 4.81 (br d, J = 5.6 Hz, 2H), 4.26-4.21 (m, 2H), 3.71-3.64 (m, |
| | | 3H), 3.20 (s, 3H), 2.90-2.70 (m, 2H), 1.93-1.74 (m, 2H), 1.19 (d, J = 6.4 Hz, 3H) ppm |
| 468 | 544.2 | 1H NMR (400 MHz, EW9897-1831-P2A, DMSO-d6) δ = 9.62 (br t, J = 5.8 Hz, 1H), |
| | | 9.36 (s, 1H), 8.63 (s, 2H), 8.54 (d, J = 1.6 Hz, 1H), 8.43 (br s, 1H), 8.27 (dd, J = 1.6, |
| | | 7.8 Hz, 1H), 7.77 (s, 1H), 7.74 (dd, J = 2.0, 7.7 Hz, 2H), 7.36 (d, J = 7.4 Hz, 1H), |
| | | 4.98 (s, 2H), 4.81 (br d, J = 5.6 Hz, 2H), 4.26-4.21 (m, 2H), 3.71-3.64 (m, 3H), |
| | | 3.20 (s, 3H), 2.87-2.70 (m, 2H), 1.93-1.75 (m, 2H), 1.19 (d, J = 6.5 Hz, 3H) |
| 469 | 530.3 | 1H NMR (400 MHz, DMSO-d6) δ = 9.57-9.54 (m, 1H), 8.98 (s, 1H), 8.53 (d, J = |
| | | 2.0 Hz, 1H), 8.38-8.37 (m, 1H), 8.26-8.24 (m, 1H), 8.04 (d, J = 9.2 Hz, 1H), 7.89- |
| | | 7.87 (m, 1H), 7.73 (d, J = 8.0 Hz, 1H), 7.46 (s, 1H), 7.37-7.34 (m, 1H), 6.65 (d, J = |
| | | 9.2 Hz, 1H), 4.98 (s, 2H), 4.73 (d, J = 5.6 Hz, 2H), 4.27-4.19 (m, 2H), 4.18-3.77 |
| | | (m, 2H), 3.73-3.63 (m, 2H), 2.75-2.68 (m, 2H), 1.89-1.77 (m, 2H), 1.69 (d, J = |
| | | 4.0 Hz, 2H) ppm |
| 470 | 574.4 | 1H NMR (400 MHz, METHANOL-d4) δ = 9.33 (s, 1H), 8.76 (d, J = 8.6 Hz, 1H), 8.67- |
| | | 8.60 (m, 2H), 8.24-8.22 (m, 1H), 7.99 (s, 1H), 7.88 (d, J = 7.4 Hz, 1H), 7.76- |
| | | 7.70 (m, 1H), 7.67 (d, J = 7.8 Hz, 1H), 6.83 (d, J = 8.4 Hz, 1H), 5.64-5.50 (m, 1H), |
| | | 5.07 (s, 2H), 4.95 (s, 2H), 4.93-4.91 (m, 1H), 4.73-4.71 (m, 1H), 4.61-4.59 (m, |
| | | 1H), 4.47-4.45 (m, 1H), 4.40-4.31 (m, 2H), 3.57-3.53 (m, 2H), 3.44-3.37 (m, |
| | | 1H), 2.92-2.87 (m, 3H), 1.19 (d, J = 6.8 Hz, 3H) ppm |
| 471 | 574.4 | 1H NMR (400 MHz, METHANOL-d4) δ = 9.34 (s, 1H), 8.76 (d, J = 8.6 Hz, 1H), 8.67- |
| | | 8.59 (m, 2H), 8.24-8.22 (m, 1H), 7.99 (s, 1H), 7.89 (d, J = 7.4 Hz, 1H), 7.76- |
| | | 7.70 (m, 1H), 7.67 (d, J = 7.8 Hz, 1H), 6.83 (d, J = 8.6 Hz, 1H), 5.64-5.50 (m, 1H), |
| | | 5.07 (s, 2H), 4.95 (s, 2H), 4.94 (br s, 1H), 4.73-4.71 (m, 1H), 4.61-4.59 (m, 1H), |
| | | 4.47-4.45 (m, 1H), 4.39-4.34 (m, 2H), 3.58-3.53 (m, 2H), 3.43-3.37 (m, 1H), |
| | | 2.89 (s, 3H), 1.19 (d, J = 6.6 Hz, 3H) ppm |
| 472 | 543.2 | 1H NMR (400 MHz, DMSO-d6) δ = 9.64 (s, 1H), 9.42 (s, 1H), 8.74-8.69 (m, 1H), |
| | | 8.68-8.64 (m, 1H), 8.54 (d, J = 1.6 Hz, 1H), 8.41 (d, J = 7.2 Hz, 1H), 8.27 (d, J = |
| | | 7.6 Hz, 1H), 7.91 (d, J = 7.6 Hz, 1H), 7.84 (s, 1H), 7.74 (d, J = 7.6 Hz, 1H), 7.35 (d, |
| | | J = 7.2 Hz, 1H), 4.98 (s, 2H), 4.83 (d, J = 5.6 Hz, 2H), 4.29-4.15 (m, 4H), 3.88- |
| | | 3.78 (m, 2H), 3.72-3.66 (m, 2H), 2.31-2.28 (m, 1H), 1.48-1.45 (m, 1H), 1.08- |
| | | 1.06 (m, 1H) ppm |
| 473 | 566.2 | 1H NMR (400 MHz, DMSO-d6) δ = 9.59 (s, 1H), 9.13 (s, 1H), 8.53 (d, J = 1.6 Hz, |
| | | 1H), 8.39 (s, 1H), 8.29-8.25 (m, 2H), 8.25-8.23 (m, 1H), 7.95 (d, J = 9.2 Hz, 1H), |
| | | 7.79 (s, 1H), 7.74 (d, J = 7.6 Hz, 1H), 7.54 (s, 1H), 7.32-6.84 (m, 1H), 4.98 (s, 2H), |
| | | 4.75 (d, J = 5.6 Hz, 2H), 4.24-4.22 (m, 2H), 4.19-4.13 (m, 2H), 3.72-3.65 (m, |
| | | 2H), 2.91-2.88 (m, 2H), 2.03-1.95 (m, 2H) ppm |
| 474 | 610.2 | 1H NMR (400 MHz, METHANOL-d4) δ = 9.31 (s, 1H), 8.70-8.63 (m, 2H), 8.62- |
| | | 8.55 (m, 1H), 8.46 (br d, J = 1.2 Hz, 1H), 8.23-8.21 (m, 1H), 7.97 (s, 1H), 7.92 (d, |
| | | J = 7.4 Hz, 1H), 7.79-7.70 (m, 2H), 6.96 (d, J = 8.6 Hz, 1H), 4.93 (s, 2H), 4.32- |
| | | 4.31 (m, 2H), 3.93-3.91 (m, 2H), 3.77-3.75 (m, 2H), 3.21 (s, 3H), 2.56-2.54 (m, |
| | | 2H), 1.68-1.65 (m, 3H), 1.29 (d, J = 6.2 Hz, 6H) ppm |
| 475 | 590.2 | 1H NMR (400 MHz, METHANOL-d4) δ = 9.33 (s, 1H), 8.72-8.56 (m, 3H), 8.24- |
| | | 8.22 (m, 1H), 8.09-7.86 (m, 3H), 7.75-7.73 (m, 1H), 6.98 (d, J = 8.4 Hz, 1H), 6.67- |
| | | 6.48 (m, 1H), 4.95 (s, 2H), 4.28-4.26 (m, 2H), 3.86-3.71 (m, 2H), 3.21-3.16 (m, |
| | | 3H), 2.58-2.56 (m, 2H), 2.29-2.11 (m, 3H), 1.31 (d, J = 6.2 Hz, 6H) ppm |
| 476 | 574.3 | 1H NMR (400 MHz, DMSO-d6) δ = 9.62-9.60 (m, 1H), 9.36 (s, 1H), 8.67-8.58 (m, |
| | | 2H), 8.54 (d, J = 1.8 Hz, 1H), 8.42-8.31 (m, 1H), 8.27-8.25 (m, 1H), 7.87-7.68 |
| | | (m, 3H), 7.36 (d, J = 7.4 Hz, 1H), 4.98 (s, 2H), 4.81 (br d, J = 5.6 Hz, 2H), 4.33- |
| | | 4.15 (m, 2H), 3.81-3.60 (m, 3H), 3.54-3.48 (m, 1H), 3.43-3.41 (m, 1H), 3.31- |
| | | 3.29 (m, 3H), 3.26 (s, 3H), 2.85-2.69 (m, 2H), 2.08-1.98 (m, 1H), 1.81-1.71 (m, 1H) ppm |
| 477 | 574.3 | 1H NMR (400 MHz, DMSO-d6) δ = 9.62-9.60 (m, 1H), 9.36 (s, 1H), 8.72-8.58 (m, |
| | | 2H), 8.54 (d, J = 1.8 Hz, 1H), 8.38 (s, 1H), 8.27-8.25 (m, 1H), 7.93-7.61 (m, 3H), |
| | | 7.37 (d, J = 7.4 Hz, 1H), 4.98 (s, 2H), 4.81 (br d, J = 5.6 Hz, 2H), 4.29-4.16 (m, |
| | | 2H), 3.74-3.63 (m, 3H), 3.55-3.49 (m, 1H), 3.46-3.42 (m, 1H), 3.30 (s, 3H), 3.26 |
| | | (s, 3H), 2.84-2.69 (m, 2H), 2.06-1.99 (m, 1H), 1.81-1.72 (m, 1H) ppm |
| 478 | 550.3 | 1H NMR (400 MHz, DMSO-d6) δ = 9.57-9.55 (m, 1H), 9.10 (s, 1H), 8.53 (d, J = |
| | | 1.8 Hz, 1H), 8.34 (s, 1H), 8.28-8.19 (m, 2H), 8.07 (d, J = 5.4 Hz, 1H), 7.88 (d, J = |
| | | 9.2 Hz, 1H), 7.73 (d, J = 7.8 Hz, 1H), 7.51 (s, 1H), 7.20 (d, J = 5.4 Hz, 1H), 4.98 (s, |
| | | 2H), 4.74 (d, J = 6.0 Hz, 2H), 4.26-4.19 (m, 2H), 4.18-4.12 (m, 2H), 3.72-3.64 |
| | | (m, 2H), 2.87-2.85 (m, 2H), 2.04-1.96 (m, 2H) ppm |
| 479 | 595.3 | 1H NMR (400 MHz, METHANOL-d4) δ = 9.31 (s, 1H), 8.61 (d, J = 1.6 Hz, 1H), 8.48 |
| | | (d, J = 8.0 Hz, 1H), 8.22-8.19 (m, 1H), 8.00 (d, J = 8.4 Hz, 1H), 7.93 (s, 1H), 7.63 |
| | | (d, J = 7.6 Hz, 1H), 7.03-6.91 (m, 3H), 5.04 (s, 2H), 4.92 (s, 2H), 4.39-4.29 (m, |
| | | 2H), 4.25-4.20 (m, 2H), 3.76-3.69 (m, 2H), 3.58-3.50 (m, 4H), 1.73-1.64 (m, 3H) ppm |
| 480 | 560.3 | 1H NMR (400 MHz, DMSO-d6) δ = 9.65-9.64 (m, 1H), 9.40 (s, 1H), 8.66 (s, 2H), |
| | | 8.55 (d, J = 1.6 Hz, 1H), 8.40 (br s, 1H), 8.28-8.26 (m, 1H), 7.92 (d, J = 7.4 Hz, 1H), |
| | | 7.82 (s, 1H), 7.79-7.74 (m, 2H), 7.09 (d, J = 8.4 Hz, 1H), 4.99 (s, 2H), 4.83 (br d, |
| | | J = 5.6 Hz, 2H), 4.26-4.21 (m, 2H), 3.73-3.67 (m, 2H), 3.43-3.42 (m 1H), 3.38 (s, |
| | | 3H), 3.20 (s, 3H), 2.73-2.72 (m, 1H), 1.23-1.22 (m, 1H), 0.95-0.88 (m, 1H) ppm |
| 481 | 557.2 | 1H NMR (400 MHz, DMSO-d6) δ = 9.70-9.55 (m, 1H), 9.19 (s, 1H), 8.50 (d, J = |
| | | 1.6 Hz, 1H), 8.36-8.26 (m, 1H), 8.25-8.17 (m, 1H), 7.73 (d, J = 7.8 Hz, 1H), 7.65- |
| | | 7.54 (m, 2H), 7.37 (d, J = 8.2 Hz, 1H), 6.71-6.64 (m, 2H), 4.97 (s, 2H), 4.76 (br d, |
| | | J = 5.4 Hz, 2H), 4.28 (br d, J = 4.2 Hz, 4H), 4.24-4.20 (m, 2H), 3.69-3.66 (m, 2H), |
| | | 1.94-1.83 (m, 1H), 0.97-0.89 (m, 2H), 0.69-0.62 (m, 2H) ppm |
| 482 | 616.3 | 1H NMR (400 MHz, DMSO-d6) δ = 9.58-9.55 (m, 1H), 9.40 (s, 1H), 8.73-8.57 (m, |
| | | 2H), 8.47 (d, J = 1.6 Hz, 1H), 8.20 (d, J = 8.0 Hz, 1H), 7.91 (d, J = 7.2 Hz, 1H), 7.81 |
| | | (s, 1H), 7.77-7.71 (m, 1H), 7.69 (d, J = 8.0 Hz, 1H), 7.03 (d, J = 8.4 Hz, 1H), 4.81 |
| | | (d, J = 5.6 Hz, 2H), 4.31 (d, J = 11.2 Hz, 2H), 3.78-3.61 (m, 3H), 3.58-3.50 (m, |
| | | 1H), 3.50-3.37 (m, 5H), 2.52 (s, 2H), 2.30-2.19 (m, 2H), 1.21 (d, J = 6.0 Hz, 6H), |
| | | 1.03-1.00 (m, 3H) ppm |
| 483 | 572.3 | 1H NMR (400 MHz, DMSO-d6) δ = 9.60-9.57 (m, 1H), 9.40 (s, 1H), 8.70-8.58 (m, |
| | | 2H), 8.45 (s, 1H), 8.43 (d, J = 1.6 Hz, 1H), 8.18 (d, J = 8.4 Hz, 1H), 7.91 (d, J = 7.6 |
| | | Hz, 1H), 7.79 (s, 1H), 7.74-7.68(m, 1H), 7.67 (d, J = 8.4 Hz, 1H), 7.03 (d, J = 8.4 |
| | | Hz, 1H), 4.80 (d, J = 5.6 Hz, 2H), 4.31 (d, J = 11.2 Hz, 2H), 3.73-3.59 (m, 3H), |
| | | 3.59-3.51 (m, 1H), 3.26 (s, 2H), 2.52 (s, 2H), 2.23-2.10 (m, 1H), 1.38 (d, J = 7.2 |
| | | Hz, 3H), 1.21 (d, J = 6.4 Hz, 6H) ppm |
| 484 | 622.2 | 1H NMR (400 MHz, DMSO-d6) δ = 9.64-9.63 (m, 1H), 9.40 (s, 1H), 8.73-8.59 (m, |
| | | 2H), 8.53-8.36 (m, 2H), 7.91 (d, J = 7.2 Hz, 1H), 7.82 (s, 1H), 7.75-7.73 (m, 1H), |
| | | 7.03 (d, J = 8.4 Hz, 1H), 4.80 (d, J = 5.2 Hz, 2H), 4.56-4.47 (m, 1H), 4.31 (d, J = |
| | | 11.6 Hz, 2H), 4.26-4.17 (m, 1H), 3.76-3.62 (m, 3H), 2.54-2.52 (m, 2H), 2.41- |
| | | 2.34 (m, 1H), 2.30-2.18 (m, 1H), 1.35 (d, J = 7.0 Hz, 3H), 1.21 (d, J = 6.0 Hz, 6H) ppm |
| 485 | 616.3 | 1H NMR (400 MHz, DMSO-d6) δ = 9.57-9.55 (m, 1H), 9.40 (s, 1H), 8.71-8.58 (m, |
| | | 2H), 8.50-8.45 (m, 1H), 8.20-8.18 (m, 1H), 7.91 (d, J = 7.4 Hz, 1H), 7.84-7.65 |
| | | (m, 3H), 7.03 (d, J = 8.2 Hz, 1H), 4.81 (br d, J = 5.6 Hz, 2H), 4.32 (br d, J = 11.6 |
| | | Hz, 2H), 3.77-3.63 (m, 3H), 3.57-3.52 (m, 1H), 3.49-3.39 (m, 5H), 2.54-2.53 |
| | | (m, 2H), 2.31-2.21 (m, 2H), 1.22 (d, J = 6.2 Hz, 6H), 1.02-1.00 (m, 3H) ppm |
| 486 | 622.2 | 1H NMR (400 MHz, DMSO-d6) δ = 9.64-9.63 (m, 1H), 9.40 (s, 1H), 8.73-8.59 (m, |
| | | 2H), 8.53-8.36 (m, 2H), 7.92 (d, J = 7.2 Hz, 1H), 7.82 (s, 1H), 7.75-7.73 (m, 1H), |
| | | 7.02 (d, J = 8.4 Hz, 1H), 4.81 (d, J = 5.2 Hz, 2H), 4.56-4.47 (m, 1H), 4.31 (d, J = |
| | | 11.6 Hz, 2H), 4.26-4.17 (m, 1H), 3.76-3.62 (m, 3H), 2.54-2.52 (m, 2H), 2.41- |
| | | 2.34 (m, 1H), 2.30-2.17 (m, 1H), 1.34 (d, J = 7.0 Hz, 3H), 1.20 (d, J = 6.0 Hz, 6H) ppm |
| 487 | 491.1 | 1H NMR (400 MHz, DMSO-d6) δ = 9.66-9.65 (m, 1H), 9.42 (s, 1H), 8.74-8.62 (m, |
| | | 2H), 8.54 (d, J = 2.0 Hz, 1H), 8.27-8.25 (m, 1H), 8.21 (d, J = 7.6 Hz, 1H), 7.92- |
| | | 7.90 (m, 1H), 7.83 (s, 1H), 7.74 (d, J = 8.0 Hz, 1H), 7.00 (d, J = 8.4 Hz, 1H), 4.98 |
| | | (s, 2H), 4.83 (d, J = 5.6 Hz, 2H), 4.26-4.19 (m, 2H), 4.04 (s, 3H), 3.72-3.64 (m, 2H) ppm |
| 488 | 530.2 | 1H NMR (400 MHz, DMSO-d6) δ = 9.63-9.61 (m, 1H), 9.35 (s, 1H), 8.62 (s, 2H), |
| | | 8.53 (d, J = 1.6 Hz, 1H), 8.26 (d, J = 7.6 Hz, 1H), 7.84-7.65 (m, 3H), 7.33 (d, J = |
| | | 7.6 Hz, 1H), 4.98 (s, 2H), 4.80 (d, J = 5.6 Hz, 2H), 4.28-4.19 (m, 2H), 3.73-3.64 |
| | | (m, 2H), 3.44-3.38 (m, 2H), 3.19 (s, 3H), 2.79-2.76 (m, 2H), 1.97-1.84 (m, 2H) ppm |
| 489 | 555.2 | 1H NMR (400 MHz, DMSO-d6) δ = 9.65-9.63 (m, 1H), 9.45 (s, 1H), 8.76 (d, J = |
| | | 8.6 Hz, 1H), 8.64 (d, J = 8.6 Hz, 1H), 8.54 (d, J = 1.6 Hz, 1H), 8.48 (s, 1H), 8.36- |
| | | 8.22 (m, 2H), 8.13 (d, J = 8.0 Hz, 1H), 7.85 (s, 1H), 7.75 (d, J = 8.0 Hz, 1H), 7.36- |
| | | 6.86 (m, 1H), 4.99 (s, 2H), 4.83 (br d, J = 5.6 Hz, 2H), 4.63-4.60 (m, 2H), 4.27- |
| | | 4.19 (m, 2H), 3.74-3.64 (m, 2H), 1.47-1.40 (m, 3H) ppm |
| 490 | 572.3 | 1H NMR (400 MHz, DMSO-d6) δ = 9.59-9.57 (m, 1H), 9.40 (s, 1H), 8.72-8.58 (m, |
| | | 2H), 8.49-8.46 (m, 1H), 8.43 (d, J = 1.8 Hz, 1H), 8.18-8.16 (m, 1H), 7.91 (d, J = |
| | | 7.4 Hz, 1H), 7.82-7.64 (m, 3H), 7.03 (d, J = 8.4 Hz, 1H), 4.80 (d, J = 5.8 Hz, 2H), |
| | | 4.31 (br d, J = 11.2 Hz, 2H), 3.79-3.48 (m, 4H), 3.29 (br s, 2H), 2.53 (br d, J = 2.0 |
| | | Hz, 2H), 2.23-2.04 (m, 1H), 1.38 (d, J = 7.2 Hz, 3H), 1.21 (d, J = 6.2 Hz, 6H) ppm |
| 491 | 532.2 | 1H NMR (400 MHz, DMSO-d6) δ = 9.63-9.50 (m, 1H), 9.08 (s, 1H), 8.53 (d, J = |
| | | 1.6 Hz, 1H), 8.44-8.39 (m, 1H), 8.32-8.22 (m, 3H), 7.81-7.69 (m, 2H), 7.48 (s, |
| | | 1H), 7.20 (s, 1H), 4.98 (s, 2H), 4.73 (d, J = 5.6 Hz, 2H), 4.32 (s, 4H), 4.27-4.19 (m, |
| | | 2H), 3.73-3.65 (m, 2H), 2.25 (s, 3H) ppm |
| 492 | 545.1 | 1H NMR (400 MHz, DMSO-d6) δ = 9.61-9.49 (m, 1H), 8.97 (s, 1H), 8.52 (d, J = |
| | | 1.8 Hz, 1H), 8.36 (s, 1H), 8.29-8.21 (m, 1H), 8.13 (d, J = 9.2 Hz, 1H), 7.73 (d, J = |
| | | 8.0 Hz, 1H), 7.44-7.33 (m, 2H), 7.21-7.10 (m, 1H), 6.93 (d, J = 8.2 Hz, 1H), 6.76 |
| | | (d, J = 8.0 Hz, 1H), 4.97 (s, 2H), 4.70 (d, J = 5.8 Hz, 2H), 4.28-4.16 (m, 2H), 4.09- |
| | | 3.99 (m, 2H), 3.82 (s, 3H), 3.74-3.64 (m, 2H), 2.69-2.63 (m, 2H), 1.93-1.81 (m, 2H) ppm |
| 493 | 539.2 | 1H NMR (400 MHz, DMSO-d6) δ = 9.63-9.61 (m, 1H), 9.47 (s, 1H), 8.73 (d, J = |
| | | 8.4 Hz, 1H), 8.51 (d, J = 1.8 Hz, 1H), 8.24-8.21 (m, 1H), 8.00-7.98 (m, 2H), 7.83 |
| | | (s, 1H), 7.72 (d, J = 7.8 Hz, 1H), 7.00 (d, J = 8.8 Hz, 1H), 4.97 (s, 2H), 4.83 (d, J = |
| | | 5.6 Hz, 2H), 4.34-4.31 (m, 2H), 4.24-4.19 (m, 2H), 3.67 (br s, 2H), 1.33-1.30 (m, 3H) ppm |
| 494 | 606.2 | 1H NMR (400 MHz, DMSO-d6) δ = 9.62-9.60 (m, 1H), 9.39 (s, 1H), 8.67-8.61 (m, |
| | | 2H), 8.28-8.27 (m, 1H), 8.23-8.20 (m, 1H), 7.90 (d, J = 7.2 Hz, 1H), 7.80 (s, 1H), |
| | | 7.76-7.74 (m, 1H), 7.04 (d, J = 8.4 Hz, 1H), 4.80 (br d, J = 5.6 Hz, 2H), 4.32-4.27 |
| | | (m, 3H), 3.69-3.65 (m, 4H), 2.53 (br d, J = 2.4 Hz, 2H), 2.30-2.18 (m, 2H), 1.45 (d, |
| | | J = 6.4 Hz, 3H), 1.22 (d, J = 6.4 Hz, 6H) ppm |
| 495 | 606.1 | 1H NMR (400 MHz, DMSO-d6) δ = 9.62-9.60 (m, 1H), 9.39 (s, 1H), 8.67-8.61 (m, |
| | | 2H), 8.28-8.27 (m, 1H), 8.23-8.20 (m, 1H), 7.91 (d, J = 7.2 Hz, 1H), 7.80 (s, 1H), |
| | | 7.76-7.74 (m, 1H), 7.05 (d, J = 8.4 Hz, 1H), 4.81 (br d, J = 5.6 Hz, 2H), 4.32-4.27 |
| | | (m, 3H), 3.69-3.65 (m, 4H), 2.53 (br d, J = 2.4 Hz, 2H), 2.30-2.18 (m, 2H), 1.45 (d, |
| | | J = 6.4 Hz, 3H), 1.21 (d, J = 6.4 Hz, 6H) ppm |
| 496 | 546.2 | 1H NMR (400 MHz, DMSO-d6) δ = 9.59-9.56 (m, 1H), 9.09 (s, 1H), 8.53 (s, 1H), |
| | | 8.24-8.21 (m, 3H), 8.02 (d, J = 9.2 Hz, 1H), 7.76-7.72 (m, 2H), 7.51 (s, 1H), 7.10- |
| | | 7.08 (m, 1 H), 4.97 (s, 2H), 4.74 (d, J = 6.0 Hz, 2H), 4.43-4.35 (m, 2H), 4.23-4.21 |
| | | (m, 2H), 3.90-3.82 (m, 1H), 3.69-3.66 (m, 2H), 3.36 (s, 3H), 2.14-2.06 (m, 2H) ppm |
| 497 | 537.2 | 1H NMR (400 MHz, DMSO-d6) δ = 9.65-9.63 (m, 1H), 9.42 (s, 1H), 8.71 (d, J = |
| | | 8.4 Hz, 1H), 8.57 (d, J = 8.8 Hz, 1H), 8.53 (d, J = 2.0 Hz, 1H), 8.47-8.41 (m, 1H), |
| | | 8.29-8.27 (m, 1H), 8.24-8.21 (m, 1H), 7.89-7.80 (m, 2H), 7.75 (d, J = 8.0 Hz, |
| | | 1H), 4.97 (s, 2H), 4.82 (d, J = 5.2 Hz, 2H), 4.62-4.59 (m, 2H), 4.30-4.27 (m, 1H), |
| | | 4.04-4.02 (m, 1H), 3.70-3.60 (m, 1H), 1.46-1.43 (m, 3H), 1.16 (d, J = 7.2 Hz, 3H) ppm |
| 498 | 587.3 | 1H NMR (400 MHz, DMSO-d6) δ = 9.39 (s, 1H), 9.28-9.26 (m, 1H), 8.69-8.60 (m, |
| | | 2H), 8.42 (s, 1H), 8.35 (d, J = 2.2 Hz, 1H), 8.07-8.06 (m, 1H), 7.92 (d, J = 7.4 Hz, |
| | | 1H), 7.78-7.70 (m, 2H), 7.03 (d, J = 8.4 Hz, 1H), 6.96 (d, J = 9.2 Hz, 1H), 4.77 (d, |
| | | J = 5.6 Hz, 2H), 4.32 (br d, J = 11.2 Hz, 2H), 3.96-3.95 (m, 1H), 3.76-3.61 (m, |
| | | 4H), 3.11 (s, 3H), 2.53 (br s, 2H), 1.27 (d, J = 6.8 Hz, 3H), 1.22 (d, J = 6.2 Hz, 6H) ppm |
| 499 | 517.2 | 1H NMR (400 MHz, DMSO-d6) δ = 9.64-9.63 (m, 1H), 9.39 (s, 1H), 8.67 (d, J = 8.8 |
| | | Hz, 1H), 8.54 (d, J = 1.6 Hz, 1H), 8.51 (d, J = 8.8 Hz, 1H), 8.27-8.25 (m, 1H), 8.14 |
| | | (d, J = 7.6 Hz, 1H), 7.80 (s, 1H), 7.75-7.70 (m, 2H), 4.98 (s, 2H), 4.82 (d, J = 6.0 |
| | | Hz, 2H), 4.36-4.34 (m, 2H), 4.24-4.21 (m, 2H), 3.69-3.67 (m, 2H), 2.88-2.85 (m, |
| | | 2H), 1.98-1.95 (m, 2H) ppm |
| 500 | 558.3 | 1H NMR (400 MHz, DMSO-d6) δ = 9.54-9.51 (m, 1H), 9.40 (s, 1H), 8.65-8.61 (m, |
| | | 2H), 8.42 (s, 1H), 8.39 (d, J = 1.2 Hz, 1H), 8.24-8.21 (m, 1H), 7.92 (d, J = 7.4 Hz, |
| | | 1H), 7.82 (s, 1H), 7.74-7.72 (m, 1H), 7.68 (d, J = 8.0 Hz, 1H), |
| | | 7.03 (d, J = 8.4 Hz, 1H), 4.81 (d, J = 5.6 Hz, 2H), 4.31 (br d, J = 11.0 Hz, 2H), 3.77- |
| | | 3.75 (m, 1H), 3.68-3.65 (m, 2H), 3.62-3.54 (m, 1H), 3.05-2.95 (m, 1H), 2.54- |
| | | 2.52 (m, 2H), 1.39 (d, J = 6.8 Hz, 3H), 1.21 (d, J = 6.4 Hz, 6H) ppm |
| 50 | 519.3 | 1H NMR (400 MHz, DMSO-d6) δ = 9.64-9.61 (m, 1H), 9.39 (s, 1H), 8.70-8.65 (m, |
| | | 1H), 8.64-8.56 (m, 1H), 8.54 (d, J = 1.8 Hz, 1H), 8.48-8.36 (m, 1H), 8.278.25 |
| | | (m, 1H), 8.12 (d, J = 7.4 Hz, 1H), 7.80 (s, 1H), 7.77-7.69 (m, 2H), 4.98 (s, 2H), |
| | | 4.82 (d, J = 5.6 Hz, 2H), 4.54 (q, J = 7.2 Hz, 2H), 4.28-4.20 (m, 2H), 3.73-3.64 |
| | | (m, 2H), 2.23 (s, 3H), 1.43-1.41 (m, 3H) ppm |
| 502 | 558.2 | 1H NMR (400 MHz, DMSO-d6) δ = 9.54-9.52 (m, 1H), 9.40 (s, 1H), 8.64-8.61 (m, |
| | | 2H), 8.39 (s, 1H), 8.24-8.21 (m, 1H), 7.92 (d, J = 7.6 Hz, 1H), 7.82 (s, 1H), 7.74- |
| | | 7.72 (m, 1H), 7.68 (d, J = 8.2 Hz, 1H), 7.03 (d, J = 8.4 Hz, 1H), 4.81 (br d, J = 5.6 |
| | | Hz, 2H), 4.31 (br d, J = 11.6 Hz, 2H), 3.82-3.53 (m, 4H), 3.00-2.98 (m, 1H), 2.64- |
| | | 2.54 (m, 2H), 1.39 (d, J = 6.8 Hz, 3H), 1.21 (d, J = 6.2 Hz, 6H) ppm |
| 503 | 558.2 | 1H NMR (400 MHz, DMSO-d6) δ = 9.59-9.56 (m, 1H), 9.08 (s, 1H), 8.52 (d, J = |
| | | 1.6 Hz, 1H), 8.29-8.23 (m, 3H), 7.79 (d, J = 2.0 Hz, 1H), 7.73 (d, J = 7.6 Hz, 1H), |
| | | 7.47 (s, 1H), 6.99 (d, J = 2.0 Hz, 1H), 4.98 (s, 2H), 4.73 (d, J = 6.0 Hz, 2H), 4.31 (s, |
| | | 4H), 4.26-4.19 (m, 2H), 3.73-3.64 (m, 2H), 1.98-1.85 (m, 1H), 1.00-0.90 (m, |
| | | 2H), 0.75-0.65 (m, 2H) ppm |
| 504 | 530.1 | 1H NMR (400 MHz, METHANOL-d4) δ = 9.58-9.55 (m, 1H), 9.06 (s, 1H), 8.53 (s, |
| | | 1H), 8.25 (d, J = 7.6 Hz, 1H), 8.16 (d, J = 8.8 Hz, 1H), 8.12 (s, 1H), 7.90 (d, J = 9.2 |
| | | Hz, 1H), 7.73 (d, J = 8.0 Hz, 1H), 7.65 (s, 1H), 7.49 (s, 1H), 7.08-7.07 (m, 1H), |
| | | 4.97 (s, 2H), 4.73 (d, J = 6.4 Hz, 1H), 4.24-4.08 (m, 4H), 3.69-3.66 (m, 2H), 2.97- |
| | | 2.96 (m, 1H), 2.12-2.07 (m, 1H), 1.67-1.62 (m, 1H), 1.30 (d, J = 6.8 Hz, 3H) ppm |
| 505 | 622.2 | 1H NMR (400 MHz, DMSO-d6) δ = 9.64-9.63 (m, 1H), 9.40 (s, 1H), 8.72-8.59 (m, |
| | | 2H), 8.44 (d, J = 2.0 Hz, 1H), 8.38 (d, J = 2.0 Hz, 1H), 7.90 (d, J = 7.2 Hz, 1H), 7.81 |
| | | (s, 1H), 7.77-7.75 (m, 1H), 7.03 (d, J = 8.4 Hz, 1H), 4.80 (d, J = 5.6 Hz, 2H), 4.31 |
| | | (d, J = 11.6 Hz, 2H), 4.28-4.20 (m, 1H), 3.74-3.62 (m, 4H), 2.54 (s, 2H), 2.39- |
| | | 2.33 (m, 1H), 2.23-2.14 (m, 1H), 1.51 (d, J = 6.4 Hz, 3H), 1.21 (d, J = 6.4 Hz, 6H) ppm |
| 506 | 622.2 | 1H NMR (400 MHz, DMSO-d6) δ = 9.64-9.62 (m, 1H), 9.39 (s, 1H), 8.71-8.59 (m, |
| | | 2H), 8.47-8.36 (m, 2H), 7.90 (d, J = 7.4 Hz, 1H), 7.83-7.70 (m, 2H), 7.03 (d, J = |
| | | 8.4 Hz, 1H), 4.80 (d, J = 5.6 Hz, 2H), 4.38-4.18 (m, 3H), 3.73-3.59 (m, 4H), 2.53 |
| | | (br d, J = 2.6 Hz, 2H), 2.40-2.33 (m, 1H), 2.23-2.15 (m, 1H), 1.51 (d, J = 6.2 Hz, |
| | | 3H), 1.21 (d, J = 6.2 Hz, 6H) ppm |
| 507 | 545.2 | 1H NMR (400 MHz, DMSO-d6) δ = 9.53-9.39 (m, 2H), 8.76-8.58 (m, 2H), 8.52 |
| | | (d, J = 1.8 Hz, 1H), 8.46 (br s, 1H), 8.36-8.34 (m, 1H), 8.26-8.24 (m, 1H), 7.99 (s, |
| | | 1H), 7.87-7.85 (m, 1H), 7.72 (d, J = 7.8 Hz, 1H), 7.47-7.45 (m, 1H), 5.67-5.52 |
| | | (m, 1H), 4.98 (s, 2H), 4.35-4.13 (m, 2H), 3.92 (d, J = 6.6 Hz, 2H), 3.73-3.60 (m, |
| | | 2H), 3.34 (s, 3H), 2.28-2.19 (m, 1H), 1.13-1.01 (m, 4H) ppm |
| 508 | 568.1 | 1H NMR (400 MHz, DMSO-d6) δ = 9.60-9.59 (m, 1H), 9.16 (s, 1H), 8.53 (d, J = |
| | | 1.6 Hz, 1H), 8.43 (s, 1H), 8.39-8.33 (m, 1H), 8.32-8.28 (m, 1H), 8.25 (d, J = 7.6 |
| | | Hz, 1H), 8.10 (d, J = 1.6 Hz, 1H), 7.73 (d, J = 8.0 Hz, 1H), 7.58-7.47 (m, 2H), 7.26- |
| | | 6.85 (m, 1H), 4.98 (s, 2H), 4.75 (d, J = 5.6 Hz, 2H), 4.43-4.38 (m, 2H), 4.38- |
| | | 4.32 (m, 2H), 4.26-4.19 (m, 2H), 3.74-3.62 (m, 2H) ppm |
| 509 | 587.2 | 1H NMR (400 MHz, DMSO-d6) δ = 9.74-9.72 (m, 1H), 9.39 (s, 1H), 9.25 (d, J = |
| | | 2.4 Hz, 1H), 8.72 (d, J = 2.2 Hz, 1H), 8.68-8.59 (m, 2H), 8.42 (s, 1H), 7.90 (d, J = |
| | | 7.4 Hz, 1H), 7.85 (s, 1H), 7.80-7.68 (m, 1H), 7.09-6.96 (m, 1H), 4.82 (br d, J = |
| | | 5.4 Hz, 2H), 4.31 (brd, J = 11.2 Hz, 2H), 3.67-3.64 (m, 2H), 3.54 (br s, 1H), 3.28- |
| | | 3.22 (m, 2H), 2.51 (br s, 2H), 2.30-2.17 (m, 1H), 2.07-1.85 (m, 2H), 1.79-1.63 |
| | | (m, 1H), 1.27-1.17 (m, 9H) ppm |
| 510 | 574.2 | 1H NMR (400 MHz, DMSO-d6) δ = 9.58-9.56 (m, 1H), 9.04 (s, 1H), 8.53 (d, J = |
| | | 1.6 Hz, 1H), 8.37 (s, 1H), 8.25 (d, J = 7.6 Hz, 1H), 8.15 (d, J = 9.2 Hz, 1H), 8.00 (d, |
| | | J = 2.0 Hz, 1H), 7.93 (d, J = 9.2 Hz, 1H), 7.73 (d, J = 8.0 Hz, 1H), 7.54-7.41 (m, |
| | | 2H), 4.98 (s, 2H), 4.73 (d, J = 5.6 Hz, 2H), 4.26-4.20 (m, 2H), 4.17-4.09 (m, 2H), |
| | | 3.73-3.64 (m, 2H), 3.54-3.52 (m, 2H), 3.25 (s, 3H), 2.80-2.78 (m, 4H), 1.99- |
| | | 1.90 (m, 2H) ppm |
| 511 | 544.1 | 1H NMR (400 MHz, DMSO-d6) δ = 9.56-9.55 (m, 1H), 9.06 (s, 1H), 8.51 (d, J = |
| | | 1.6 Hz, 1H), 8.41 (s, 1H), 8.30-8.22 (m, 2H), 7.73 (d, J = 8.0 Hz, 1H), 7.58-7.51 |
| | | (m, 1H), 7.48 (s, 1H), 7.41 (d, J = 9.2 Hz, 1H), 7.37-7.27 (m, 2H), 7.21-7.13 (m, |
| | | 1H), 4.98 (s, 2H), 4.80 (s, 2H), 4.72 (d, J = 5.6 Hz, 2H), 4.27-4.18 (m, 2H), 3.72- |
| | | 3.64 (m, 2H), 3.34-3.33 (m, 3H) ppm |
| 512 | 530.2 | 1H NMR (400 MHz, DMSO-d6) δ = 9.57-9.56 (m, 1H), 8.96 (s, 1H), 8.53 (d, J = |
| | | 1.8 Hz, 1H), 8.32 (s, 1H), 8.26-8.25 (m, 1H), 8.14 (d, J = 9.2 Hz, 1H), 7.74 (d, J = |
| | | 7.8 Hz, 1H), 7.38-7.35 (m, 2H), 7.22 (m, 1H), 7.09-7.02 (m, 1H), 6.81-6.80 (m, |
| | | 1H), 6.67-6.61 (m, 1H), 4.98 (s, 2H), 4.70 (d, J = 5.8 Hz, 2H), 4.23-4.21 (m, 4H), |
| | | 3.72-3.66 (m, 2H), 3.36-3.35 (m, 2H), 2.91 (s, 3H) ppm |
| 513 | 550.1 | 1H NMR (400 MHz, DMSO-d6) δ = 9.59-9.57 (m, 1H), 9.12 (s, 1H), 8.53 (d, J = |
| | | 1.8 Hz, 1H), 8.48-8.39 (m, 1H), 8.33-8.21 (m, 2H), 7.93 (d, J = 9.2 Hz, 1H), 7.73 |
| | | (d, J = 7.8 Hz, 1H), 7.63 (d, J = 7.7 Hz, 1H), 7.52 (s, 1H), 7.08 (d, J = 7.8 Hz, 1H), |
| | | 4.98 (s, 2H), 4.75 (d, J = 5.8 Hz, 2H), 4.28-4.19 (m, 2H), 4.18-4.07 (m, 2H), 3.72- |
| | | 3.64 (m, 2H), 2.82-2.80 (m, 2H), 2.01-1.92 (m, 2H) ppm |
| 514 | 530.2 | 1H NMR (400 MHz, DMSO-d6) δ = 9.49-9.48 (m, 1H), 9.03 (s, 1H), 8.46 (d, J = |
| | | 2.4 Hz, 1H), 8.25 (d, J = 2.4 Hz, 1H), 8.14 (d, J = 9.2 Hz, 1H), 7.98 (d, J = 1.6 Hz, |
| | | 1H), 7.91 (d, J = 9.2 Hz, 1H), 7.46-7.42 (m, 2H), 7.38 (d, J = 8.4 Hz, 1H), 4.71 (d, |
| | | J = 5.6 Hz, 2H), 4.33-4.23 (m, 2H), 4.16-4.07 (m, 2H), 3.66-3.58 (m, 2H), 2.80- |
| | | 2.78 (m, 2H), 2.31-2.26 (m, 2H), 2.25 (s, 3H), 1.99-1.89 (m, 2H) ppm |
| 515 | 587.3 | 1H NMR (400 MHz, DMSO-d6) δ = 9.75-9.73 (m, 1H), 9.40 (s, 1H), 9.26 (d, J = |
| | | 2.2 Hz, 1H), 8.73 (d, J = 2.0 Hz, 1H), 8.69-8.56 (m, 2H), 7.91 (d, J = 7.4 Hz, 1H), |
| | | 7.86 (s, 1H), 7.75 (t, J = 8.0 Hz, 1H), 7.03 (d, J = 8.6 Hz, 1H), 4.83 (br d, J = 5.8 Hz, |
| | | 2H), 4.31 (brd, J = 11.4 Hz, 2H), 3.79-3.62 (m, 2H), 3.60-3.43 (m, 1H), 3.30 (br |
| | | s, 2H), 2.54-2.52 (m, 2H), 2.30-2.18 (m, 1H), 2.12-1.90 (m, 2H), 1.82-1.60 (m, |
| | | 1H), 1.26-1.10 (m, 9H) ppm |
| 516 | 606.2 | 1H NMR (400 MHz, DMSO-d6) δ = 9.70-9.67 (m, 1H), 9.40 (s, 1H), 8.72-8.58 (m, |
| | | 2H), 8.45 (d, J = 1.6 Hz, 1H), 8.18-8.15 (m, 1H), 7.90 (d, J = 7.6 Hz, 1H), 7.82 (s, |
| | | 1H), 7.78-7.69 (m, 1H), 7.03 (d, J = 8.4 Hz, 1H), 5.53-5.48 (m, 1H), 4.82 (d, J = |
| | | 5.6 Hz, 2H), 4.31 (d, J = 11.2 Hz, 2H), 4.27-4.20 (m, 1H), 4.03-3.85 (m, 2H), 3.76- |
| | | 3.63 (m, 3H), 2.53-2.52 (m, 2H), 1.62-1.60 (m, 3H), 1.21 (d, J = 6.0 Hz, 6H) ppm |
| 517 | 602.2 | 1H NMR (400 MHz, DMSO-d6) δ = 9.66-9.63 (m, 1H), 9.40 (s, 1H), 8.71-8.57 (m, |
| | | 3H), 8.32-8.29 (m, 1H), 7.96-7.87 (m, 1H), 7.83-7.71 (m, 3H), 7.03 (d, J = 8.4 |
| | | Hz, 1H), 5.32-5.27 (m, 1H), 4.82 (d, J = 5.6 Hz, 2H), 4.31 (d, J = 11.6 Hz, 2H), |
| | | 4.21-4.11 (m, 1H), 4.00-3.94 (m, 1H), 3.72-3.63 (m, 2H), 3.61-3.49 (m, 1H), |
| | | 2.53-2.52 (m, 2H), 1.62 (d, J = 6.4 Hz, 3H), 1.26-1.19 (m, 9H) ppm |
| 518 | 602.4 | 1H NMR (400 MHz, DMSO-d6) δ = 9.65-9.63 (m, 1H), 9.40 (s, 1H), 8.71-8.56 (m, |
| | | 3H), 8.43-8.36 (m, 1H), 8.32-8.29 (m, 1H), 7.91 (d, J = 7.6 Hz, 1H), 7.82-7.71 |
| | | (m, 3H), 7.02 (d, J = 8.4 Hz, 1H), 5.30 (d, J = 6.4 Hz, 1H), 4.82 (d, J = 5.6 Hz, 2H), |
| | | 4.31 (br d, J = 11.6 Hz, 2H), 4.21-4.12 (m, 1H), 4.00-3.94 (m, 1H), 3.69-3.65 |
| | | (m, 2H), 3.56-3.53 (m, 1H), 2.53 (br s, 2H), 1.62 (d, J = 6.8 Hz, 3H), 1.25-1.19 |
| | | (m, 9H) ppm |
| 519 | 555.3 | 1H NMR (400 MHz, DMSO-d6) δ = 9.66-9.63 (m, 1H), 9.45 (s, 1H), 8.80-8.65 (m, |
| | | 2H), 8.53 (d, J = 1.6 Hz, 1H), 8.46 (s, 1H), 8.33 (d, J = 7.6 Hz, 1H), 8.29-8.27 (m, |
| | | 1H), 8.14 (d, J = 8.0 Hz, 1H), 7.88 (s, 1H), 7.75 (d, J = 7.6 Hz, 1H), 7.30-7.02 (m, |
| | | 1H), 4.97 (s, 2H), 4.84 (d, J = 5.6 Hz, 2H), 4.30-4.27 (m, 1H), 4.14 (s, 3H), 4.04- |
| | | 3.99 (m, 1H), 3.71-3.60 (m, 1H), 1.17 (d, J = 6.8 Hz, 3H) ppm |
| 520 | 567.3 | 1H NMR (400 MHz, DMSO-d6) δ = 9.58-9.52 (m, 1H), 9.08 (s, 1H), 8.51 (d, J = |
| | | 1.8 Hz, 1H), 8.40 (s, 2H), 8.32 (d, J = 9.0 Hz, 1H), 8.24 (dd, J = 1.9, 7.8 Hz, 1H), |
| | | 7.72 (d, J = 7.9 Hz, 1H), 7.63 (d, J = 8.5 Hz, 1H), 7.58 (d, J = 9.0 Hz, 1H), 7.45 (s, |
| | | 1H), 7.14 (s, 1H), 7.12-6.81 (m, 2H), 4.99-4.97 (m, 2H), 4.72 (d, J = 6.0 Hz, 2H), |
| | | 4.36-4.30 (m, 2H), 4.24-4.20 (m, 4H), 3.69-3.66 (m, 2H) ppm |
| 521 | 566.2 | 1H NMR (400 MHz, DMSO-d6) δ = 9.57-9.55 (m, 1H), 9.10 (s, 1H), 8.53 (d, J = |
| | | 1.8 Hz, 1H), 8.28-8.19 (m, 3H), 7.84 (d, J = 9.2 Hz, 1H), 7.73 (d, J = 7.8 Hz, 1H), |
| | | 7.52 (s, 1H), 7.40-7.12 (m, 2H), 4.98 (s, 2H), 4.74 (d, J = 5.8 Hz, 2H), 4.23-4.20 |
| | | (m, 2H), 4.18-4.12 (m, 2H), 3.72-3.61 (m, 2H), 2.92-2.90 (m, 2H), 1.99-1.97 (m, 2H) ppm |
| 522 | 606.2 | 1H NMR (400 MHz, DMSO-d6) δ = 9.69-9.68 (m, 1H), 9.40 (s, 1H), 8.69-8.60 (m, |
| | | 2H), 8.45 (d, J = 1.2 Hz, 1H), 8.17-8.15 (m, 1H), 7.90 (d, J = 7.2 Hz, 1H), 7.82 (s, |
| | | 1H), 7.78-7.72 (m, 1H), 7.03 (d, J = 8.4 Hz, 1H), 5.53-5.48 (m, 1H), 4.82 (d, J = |
| | | 5.6 Hz, 2H), 4.31 (d, J = 11.2 Hz, 2H), 4.27-4.20 (m, 1H), 4.02-3.86 (m, 2H), 3.76- |
| | | 3.61 (m, 3H), 2.53-2.52 (m, 2H), 1.62-1.60 (m, 3H), 1.21 (d, J = 6.4 Hz, 6H) ppm |
| 523 | 532.2 | 1H NMR (400 MHz, DMSO-d6) δ = 9.58-9.56 (m, 1H), 9.11 (s, 1H), 8.52 (d, J = |
| | | 1.6 Hz, 1H), 8.34-8.24 (m, 3H), 8.24-8.23 (m, 1H), 7.92-7.90 (m, 1H), 7.74 (d, |
| | | J = 8.9 Hz, 1H), 7.53 (s, 1H), 7.35-7.33 (m, 1H), 7.08-7.05 (m, 1H), 5.04-4.88 (m, |
| | | 2H), 4.74 (br d, J = 5.8 Hz, 2H), 4.35 (s, 4H), 4.29-4.27 (m, 1H), 4.02-4.00 (m, |
| | | 1H), 3.70-3.60 (m, 1H), 1.17 (d, J = 7.0 Hz, 3H) ppm |
| 524 | 550.1 | 1H NMR (400 MHz, DMSO-d6) δ = 9.65-9.63 (m, 1H), 9.10 (s, 1H), 8.38 (d, J = |
| | | 1.4 Hz, 1H), 8.35-8.26 (m, 2H), 8.17-8.15 (m, 1H), 7.91-7.90 (m, 1H), 7.53 (s, |
| | | 1H), 7.34-7.32 (m, 1H), 7.07-7.05 (m, 1H), 5.10-4.90 (m, 2H), 4.73 (br d, J = 5.4 |
| | | Hz, 2H), 4.34 (s, 4H), 4.28-4.25 (m, 1H), 4.02-4.00 (m, |
| | | 1H), 3.73-3.71 (m, 1H), 1.17 (d, J = 7.0 Hz, 3H) ppm |
| 525 | 583.1 | 1H NMR (400 MHz, DMSO-d6) δ = 9.57-9.54 (m, 1H), 9.03 (s, 1H), 8.50 (d, J = 1.6 |
| | | Hz, 1H), 8.40 (br d, J = 2.4 Hz, 1H), 8.26-8.21 (m, 2H), 7.74-7.70 (m, 2H), 7.44- |
| | | 7.37(m, 4H), 4.96 (s, 2H), 4.70 (d, J = 5.6 Hz, 2H), 4.23-4.20 (m, 2H), 4.00-3.97 (m, |
| | | 2H), 3.67-3.65 (m, 2H), 2.82-2.79 (m, 2H), 1.98-1.92(m, 2H) ppm |
| 526 | 534.1 | 1H NMR (400 MHz, DMSO-d6) δ = 9.70-9.48 (m, 1H), 9.10 (s, 1H), 8.52 (d, J = |
| | | 2.0 Hz, 1H), 8.44-8.36 (m, 1H), 8.30-8.21 (m, 2H), 8.17-8.08 (m, 1H), 7.96 (d, |
| | | J = 9.2 Hz, 1H), 7.73 (d, J = 8.0 Hz, 1H), 7.51 (s, 1H), 7.03-6.92 (m, 1H), 4.98 (s, |
| | | 2H), 4.74 (d, J = 6.0 Hz, 2H), 4.29-4.12 (m, 4H), 3.74-3.62 (m, 2H), 2.87-2.76 |
| | | (m, 2H), 2.03-1.91 (m, 2H) ppm |
| 527 | 540.2 | 1H NMR (400 MHz, METHANOL-d4) δ = 9.02 (s, 1H), 8.62 (d, J = 1.8 Hz, 1H), 8.51- |
| | | 8.43 (m, 1H), 8.26-8.18 (m, 2H), 7.67-7.63 (m, 2H), 7.60 (s, 1H), 7.54-7.47 (m, |
| | | 3H), 5.06 (s, 2H), 4.85 (br s, 2H), 4.43-4.33 (m, 2H), 4.10-4.08 (m, 2H), 3.59- |
| | | 3.51 (m, 2H), 2.86-2.84 (m, 2H), 2.15-2.01 (m, 2H) ppm |
| 528 | 585.2 | 1H NMR (400 MHz, DMSO-d6) δ = 9.71-9.66 (m, 1H), 9.40 (s, 1H), 9.14 (d, J = |
| | | 2.0 Hz, 1H), 8.70-8.60 (m, 3H), 7.91 (d, J = 7.2 Hz, 1H), 7.83 (s, 1H), 7.77-7.75 |
| | | (m, 1H), 7.04 (d, J = 8.4 Hz, 1H), 4.83 (br d, J = 5.6 Hz, 2H), 4.32 (br d, J = 11.6 |
| | | Hz, 2H), 3.81-3.75 (m, 2H), 3.69-3.67 (m, 2H), 2.58-2.53 (m, 4H), 1.99-1.88 |
| | | (m, 1H), 1.72-1.67 (m, 1H), 1.54-1.48 (m, 1H), 1.22 (d, J = 6.4 Hz, 6H), 0.74- |
| | | 0.67 (m, 1H) ppm |
| 529 | 531.1 | 1H NMR (400 MHz, DMSO-d6) δ = 9.64-9.63 (m, 1H), 9.39 (s, 1H), 8.66(d, J = 8.4 |
| | | Hz, 1H), 8.54-8.50 (m, 2H), 8.27-8.25 (m, 1H), 8.14 (d, J = 7.6 Hz, 1H), 7.80 (s, |
| | | 1H), 7.75-7.70 (m, 2H), 4.98 (s, 2H), 4.82 (br d, J = 6.0 Hz, 2H), 4.41-4.39 (m, 1H), |
| | | 4.24-4.21 (m, 2H), 3.69-3.67 (m, 2H), 2.92-2.84 (m, 2H), 2.08-2.03 (m, 1H), 1.71- |
| | | 1.66 (m, 1H), 1.42 (d, J = 6.4 Hz, 3H) ppm |
| 530 | 573.1 | 1H NMR (400 MHz, DMSO-d6) δ = 9.75-9.73 (m, 1H), 9.45 (s, 1H), 8.78-8.73 (m, |
| | | 1H), 8.71-8.66 (m, 1H), 8.47-8.43 (m, 1H), 8.41 (d, J = 1.2 Hz, 1H), 8.33 (d, J = |
| | | 8.0 Hz, 1H), 8.22-8.19 (m, 1H), 8.15 (d, J = 8.0 Hz, 1H), 7.90 (s, 1H), 7.31-7.01 |
| | | (m, 1H), 5.11-5.03 (m, 1H), 5.00-4.93 (m, 1H), 4.84 (d, J = 5.2 Hz, 2H), 4.31- |
| | | 4.27 (m, 1H), 4.14 (s, 3H), 4.05-4.00 (m, 1H), 3.79-3.70 (m, 1H), 1.18 (d, J = 7.2 |
| | | Hz, 3H) ppm |
| 53- | 584.3 | 1H NMR (400 MHz, DMSO-d6) δ = 9.62-9.51 (m, 1H), 9.39 (s, 1H), 8.64 (q, J = |
| | | 8.6 Hz, 2H), 8.42 (d, J = 1.8 Hz, 1H), 8.15-8.13 (m, 1H), 7.91 (d, J = 7.4 Hz, 1H), |
| | | 7.79 (s, 1H), 7.75-7.73 (m, 1H), 7.57 (d, J = 8.2 Hz, 1H), 7.03 (d, J = 8.6 Hz, 1H), |
| | | 4.80 (d, J = 5.4 Hz, 2H), 4.31 (br d, J = 12.4 Hz, 2H), 3.68-3.66 (m, 2H), 3.16- |
| | | 3.14 (m, 2H), 2.52 (br s, 2H), 2.37-2.34 (m, 2H), 1.36-1.29 (m, 2H), 1.21 (d, J = |
| | | 6.2 Hz, 6H), 1.17 (br d, J = 2.0 Hz, 2H) ppm |
| 532 | 565.2 | 1H NMR (400 MHz, DMSO-d6) δ = 9.56-9.54 (m, 1H), 9.02 (s, 1H), 8.52 (d, J = |
| | | 1.6 Hz, 1H), 8.38 (br s, 1H), 8.27-8.18 (m, 2H), 7.73 (d, J = 7.8 Hz, 1H), 7.58 (s, |
| | | 1H), 7.43-7.36 (m, 3H), 7.25 (d, J = 7.6 Hz, 1H), 7.12-6.79 (m, 1H), 4.97 (s, 2H), |
| | | 4.71 (d, J = 5.6 Hz, 2H), 4.28-4.17 (m, 2H), 4.00 (t, J = 6.4 Hz, 2H), 3.75-3.63 |
| | | (m, 2H), 2.79-2.77 (m, 2H), 1.94-1.92 (m, 2H) ppm |
| 533 | 549.1 | 1H NMR (400 MHz, METHANOL-d4) δ = 8.94 (s, 1H), 8.60 (d, J = 1.6 Hz, 1H), 8.20- |
| | | 8.18 (m, 1H), 8.14 (d, J = 9.2 Hz, 1H), 7.63 (d, J = 7.8 Hz, 1H), 7.59 (s, 1H), 7.45 |
| | | (d, J = 9.2 Hz, 1H), 7.37 (d, J = 2.0 Hz, 1H), 7.21 (d, J = 8.4 Hz, 1H), 7.07-7.05 |
| | | (m, 1H), 5.04 (s, 2H), 4.82 (s, 2H), 4.37-4.31 (m, 2H), 4.05-4.03 (m, 2H), 3.55- |
| | | 3.49 (m, 2H), 2.76-2.75 (m, 2H), 2.02-2.00 (m, 2H) ppm |
| 534 | 560.1 | 1H NMR (400 MHz, DMSO-d6) δ = 9.63-9.60 (m, 1H), 9.38 (s, 1H), 8.65(s, 2H), |
| | | 8.53(d, J = 1.6 Hz, 1H), 8.30 (s, 1H), 8.27-8.25(m, 1H), 7.89 (d, J = 7.2 Hz, 1H), |
| | | 7.81 (s, 1H), 7.74-7.69(m, 2H), 7.09 (d, J = 8.4 Hz, 1H), 4.97 (s, 2H), 4.82 (br d, J = |
| | | 5.6 Hz, 2H), 4.23-4.21 (m, 2H), 3.69-3.66 (m, 2H), 3.53-3.50 (m, 2H), 3.25(s, 3H), |
| | | 3.23 (s, 3H), 2.67-2.64 (m, 1H), 1.13-1.07 (m, 1H), 0.80-0.76 (m, 1H) ppm |
| 535 | 602.3 | 1H NMR (400 MHz, DMSO-d6) δ = 9.65-9.62 (m, 1H), 9.40 (s, 1H), 8.72-8.58 (m, |
| | | 2H), 8.52 (s, 1H), 8.48-8.37 (m, 1H), 8.36-8.27 (m, 1H), 7.91 (d, J = 7.6 Hz, 1H), |
| | | 7.83 (s, 1H), 7.81-7.68 (m, 2H), 7.03 (d, J = 8.6 Hz, 1H), 5.35-5.22 (m, 1H), 4.91- |
| | | 4.74 (m, 2H), 4.43 (d, J = 13.2 Hz, 1H), 4.31 (d, J = 12.4 Hz, 2H), 4.04-4.00 (m, |
| | | 1H), 3.72-3.60 (m, 3H), 2.53 (d, J = 4.0 Hz, 2H), 1.63 (d, J = 6.4 Hz, 3H), 1.21 (d, |
| | | J = 6.0 Hz, 6H), 1.07 (d, J = 6.8 Hz, 3H) ppm |
| 536 | 556.3 | 1H NMR (400 MHz, DMSO-d6) δ = 9.57-9.54 (m, 1H), 9.06 (s, 1H), 8.53 (s, 1H), |
| | | 8.29 (s, 1H), 8.25 (d, J = 7.6 Hz, 1H), 8.17 (d, J = 9.2 Hz, 1H), 7.82 (d, J = 9.6 Hz, |
| | | 1H), 7.73 (d, J = 7.6 Hz, 1H), 7.47 (s, 1H), 7.43 (d, J = 7.6 Hz, 1H), 6.96 (d, J = 7.6 |
| | | Hz, 1H), 4.97 (s, 2H), 4.73 (d, J = 5.6 Hz, 2H), 4.30-4.17 (m, 2H), 4.10-4.07 (m, |
| | | 2H), 3.74-3.60 (m, 2H), 2.77-2.73 (m, 2H), 2.03-1.88 (m, 3H), 0.87-0.74 (m, 4H) ppm |
| 537 | 588.3 | 1H NMR (400 MHz, DMSO-d6) δ = 9.60-9.50 (m, 1H), 9.40 (s, 1H), 8.71-8.58 (m, |
| | | 2H), 8.47 (d, J = 2.2 Hz, 1H), 8.44 (s, 1H), 8.34-8.32 (m, 1H), 7.90 (d, J = 7.4 Hz, |
| | | 1H), 7.81-7.71 (m, 2H), 7.63 (d, J = 8.6 Hz, 1H), 7.03 (d, J = 8.4 Hz, 1H), 4.80 (d, |
| | | J = 5.6 Hz, 2H), 4.40-4.25 (m, 4H), 3.76-3.61 (m, 2H), 3.45-3.40 (m, 2H), 2.52 |
| | | (br s, 2H), 2.13-1.96 (m, 2H), 1.67-1.56 (m, 2H), 1.21 (d, J = 6.2 Hz, 6H) ppm |
| 538 | 530.2 | 1H NMR (400 MHz, DMSO-d6) δ = 9.50-9.48 (m, 1H), 9.03 (s, 1H), 8.47 (d, J = |
| | | 2.4 Hz, 1H), 8.23 (d, J = 6.0 Hz, 1H), 8.14 (d, J = 7.2 Hz, 1H), 7.99 (d, J = 8.8 Hz, |
| | | 1H), 7.47-7.44 (m, 2H), 7.38 (d, J = 8.4 Hz, 1H), 6.89 (d, J = 7.6 Hz, 1H), 4.71 (d, |
| | | J = 6.0 Hz, 2H), 4.27-4.25 (m, 2H), 4.11-4.09 (m, 2H), 3.63-3.61 (m, 2H), 2.77- |
| | | 2.74 (m, 2H), 2.34 (br s, 2H), 2.28-2.26 (m, 2H), 1.94-1.91 (m, 2H) ppm |
| 539 | 545.2 | 1H NMR (400 MHz, DMSO-d6) δ = 9.58-9.53 (m, 1H), 8.99 (s, 1H), 8.52 (d, J = |
| | | 1.2 Hz, 1H), 8.34 (s, 1H), 8.25-8.23 (m, 1H), 8.15 (d, J = 9.6 Hz, 1H), 7.73 (d, J = |
| | | 8.0 Hz, 1H), 7.47 (d, J = 9.6 Hz, 1H), 7.39 (s, 1H), 7.15 (d, J = 8.4 Hz, 1H), 6.91 (d, |
| | | J = 2.4 Hz, 1H), 6.69-6.68 (m, 1H), 4.97 (s, 2H), 4.70 (d, J = 5.6 Hz, 2H), 4.27- |
| | | 4.19 (m, 2H), 3.99-3.96 (m, 2H), 3.69 (s, 5H), 2.67-2.66 (m, 2H), 1.94-1.85 (m, 2H) ppm |
| 540 | 602.3 | 1H NMR (400 MHz, DMSO-d6) δ = 9.65-9.63 (m, 1H), 9.40 (s, 1H), 8.71-8.60 (m, |
| | | 2H), 8.52 (d, J = 1.6 Hz, 1H), 8.38-8.34 (m, 1H), 8.32-8.30 (m, 1H), 7.92 (d, J = |
| | | 7.6 Hz, 1H), 7.83 (s, 1H), 7.81-7.70 (m, 2H), 7.03 (d, J = 8.4 Hz, 1H), 5.30-5.27 |
| | | (m, 1H), 4.89-4.74 (m, 2H), 4.47-4.39 (m, 1H), 4.31 (d, J = 11.6 Hz, 2H), 4.04- |
| | | 4.03 (m, 1H), 3.72-3.60 (m, 3H), 2.62-2.57 (m, 2H), 1.63 (d, J = 6.4 Hz, 3H), 1.21 |
| | | (d, J = 6.4 Hz, 6H), 1.07 (d, J = 6.8 Hz, 3H) ppm |
| 541 | 602.3 | 1H NMR (400 MHz, DMSO-d6) δ = 9.51-9.49 (m, 1H), 9.39 (s, 1H), 8.71-8.56 (m, |
| | | 2H), 8.40 (br s, 1H), 8.28 (d, J = 2.0 Hz, 1H), 8.20 (d, J = 1.6 Hz, 1H), 7.90 (d, J = |
| | | 7.4 Hz, 1H), 7.81-7.70 (m, 2H), 7.03 (d, J = 8.5 Hz, 1H), 4.79 (br d, J = 5.6 Hz, |
| | | 2H), 4.31 (brd, J = 11.8 Hz, 2H), 4.18-4.06 (m, 1H), 3.72-3.63 (m, 2H), 3.61- |
| | | 3.53 (m, 2H), 2.53-2.52 (m, 2H), 2.35 (s, 3H), 2.32-2.24 (m, 1H), 2.14 (br d, J = |
| | | 15.2 Hz, 1H), 1.48 (d, J = 6.4 Hz, 3H), 1.21 (d, J = 6.4 Hz, 6H) ppm |
| 542 | 569.1 | 1H NMR (400 MHz, DMSO-d6) δ = 9.64-9.63 (m, 1H), 9.42 (s, 1H), 8.65 (d, J = |
| | | 8.8 Hz, 1H), 8.52 (d, J = 1.8 Hz, 1H), 8.45 (d, J = 7.8 Hz, 1H), 8.43 (br s, 1H), 8.27- |
| | | 8.26 (m, 1H), 8.22 (d, J = 8.8 Hz, 1H), 7.83 (s, 1H), 7.74 (d, J = 7.8 Hz, 1H), 7.47 |
| | | (d, J = 7.8 Hz, 1H), 4.96 (s, 2H), 4.82 (br d, J = 5.4 Hz, 2H), 4.29-4.28 (m, 1H), |
| | | 4.09-3.93 (m, 4H), 3.69-3.59 (m, 1H), 2.05-2.03 (m, 3H), 1.16 (d, J = 7.2 Hz, 3H) ppm |
| 543 | 587.1 | 1H NMR (400 MHz, DMSO-d6) δ = 9.72-9.70 (m, 1H), 9.43 (s, 1H), 8.65 (d, J = |
| | | 8.4 Hz, 1H), 8.45 (d, J = 7.8 Hz, 1H), 8.39 (s, 1H), 8.25-8.12 (m, 2H), 7.86 (s, 1H), |
| | | 7.47 (d, J = 7.8 Hz, 1H), 5.16-4.91 (m, 2H), 4.82 (br d, J = 4.6 Hz, 2H), 4.41-4.22 |
| | | (m, 1H), 4.12-3.93 (m, 4H), 3.79-3.67 (m, 1H), 2.06-2.04 (m, 3H), 1.21-1.13 (m, 3H) ppm |
| 544 | 602.4 | 1H NMR (400 MHz, DMSO-d6) δ = 9.519.49 (m, 1H), 9.39 (s, 1H), 8.72-8.57 (m, |
| | | 2H), 8.28 (d, J = 2.0 Hz, 1H), 8.20 (d, J = 1.6 Hz, 1H), 7.90 (d, J = 7.4 Hz, 1H), 7.80- |
| | | 7.69 (m, 2H), 7.03 (d, J = 8.6 Hz, 1H), 4.79 (br d, J = 5.6 Hz, 2H), 4.31 (br d, J = |
| | | 11.6 Hz, 2H), 4.19-4.02 (m, 1H), 3.74-3.63 (m, 2H), 3.61-3.54 (m, 2H), 2.54 (s, |
| | | 2H), 2.38-2.26 (m, 4H), 2.14 (br d, J = 14.6 Hz, 1H), 1.48 (d, J = 6.2 Hz, 3H), 1.21 |
| | | (d, J = 6.2 Hz, 6H) ppm |
| 545 | 546.3 | 1H NMR (400 MHz, DMSO-d6) δ = 9.58-9.57 (m, 1H), 9.08 (s, 1H), 8.51 (d, J = |
| | | 1.6 Hz, 1H), 8.44 (d, J = 1.6 Hz, 1H), 8.33-8.22 (m, 3H), 7.82-7.70 (m, 2H), 7.50 |
| | | (s, 1H), 7.20 (d, J = 2.0 Hz, 1H), 4.96 (s, 2H), 4.73 (d, J = 5.6 Hz, 2H), 4.35-4.26 |
| | | (m, 5H), 4.02 (d, J = 6.0 Hz, 1H), 3.70-3.61 (m, 1H), 2.25 (s, 3H), 1.19-1.12 (m, 3H) ppm |
| 546 | 517.3 | 1H NMR (400 MHz, METHANOL-d4) δ = 8.87 (s, 1H), 8.60 (d, J = 1.6 Hz, 1H), 8.49- |
| | | 8.39 (m, 3H), 8.22-8.16 (m, 2H), 7.63 (d, J = 7.6 Hz, 1H), 7.51 (s, 1H), 7.40 (d, |
| | | J = 9.2 Hz, 1H), 5.11 (s, 2H), 5.05 (s, 2H), 4.80 (s, 2H), 4.35-4.33 (m, 2H), 4.25- |
| | | 4.23 (m, 2H), 3.55-3.51 (m, 2H), 3.18-3.15 (m, 2H) ppm |
| 547 | 622 | 1H NMR (400 MHz, METHANOL-d4) δ = 9.30 (s, 1H), 8.70-8.61 (m, 2H), 8.61- |
| | | 8.56 (m, 1H), 8.48 (s, 1H), 8.34 (d, J = 2.0 Hz, 1H), 8.00-7.88 (m, 2H), 7.75-7.71 |
| | | (m, 1H), 6.96 (d, J = 8.4 Hz, 1H), 5.69-5.64 (m, 1H), 4.92 (s, 2H), 4.41-4.27 (m, |
| | | 3H), 3.94-3.83 (m, 2H), 3.82-3.73 (m, 2H), 3.64-3.59 (m, 1H), 2.58-2.52 (m, |
| | | 2H), 1.69 (d, J = 6.8 Hz, 3H), 1.29 (d, J = 6.4 Hz, 6H) ppm |
| 548 | 547.1 | 1H NMR (400 MHz, DMSO-d6) δ = 9.61-9.48 (m, 1H), 9.00 (s, 1H), 8.51 (d, J = |
| | | 1.6 Hz, 1H), 8.38 (s, 1H), 8.29-8.17 (m, 2H), 7.73 (d, J = 7.6 Hz, 1H), 7.46 (d, J = |
| | | 8.8 Hz, 1H), 7.41-7.31 (m, 2H), 6.60-6.46 (m, 2H), 4.97 (s, 2H), 4.69 (d, J = 5.6 |
| | | Hz, 2H), 4.32-4.18 (m, 6H), 3.73 (s, 3H), 3.71-3.65 (m, 2H) ppm |
| 549 | 546.3 | 1H NMR (400 MHz, DMSO-d6) δ = 9.53-9.51 (m, 1H), 9.09 (s, 1H), 8.43-8.35 (m, |
| | | 1H), 8.34-8.22 (m, 2H), 8.16 (d, J = 1.2 Hz, 1H), 7.78 (s, 1H), 7.48 (s, 1H), 7.21 (d, |
| | | J = 1.2 Hz, 1H), 5.06 (s, 2H), 4.73 (br d, J = 5.8 Hz, 2H), 4.33 (s, 4H), 4.25-4.17 |
| | | (m, 2H), 3.70-3.63 (m, 2H), 2.50-2.50 (m, 3H), 2.26 (s, 3H) ppm |
| 550 | 550.2 | 1H NMR (400 MHz, DMSO-d6) δ = 9.72-9.58 (m, 1H), 9.09 (s, 1H), 8.44-8.39 (m, |
| | | 1H), 8.33-8.24 (m, 2H), 8.17-8.16 (m, 1H), 7.80-7.76 (m, 1H), 7.50 (s, 1H), 7.21 |
| | | (d, J = 1.2 Hz, 1H), 5.03 (s, 2H), 4.74 (br d, J = 5.6 Hz, 2H), 4.38-4.30 (m, 4H), |
| | | 4.27-4.19 (m, 2H), 3.80-3.74 (m, 2H), 2.26 (s, 3H) ppm |
| 551 | 531.2 | 1H NMR (400 MHz, DMSO-d6) δ = 9.74-9.72 (m, 1H), 9.26 (s, 1H), 8.69 (d, J = |
| | | 2.4 Hz, 1H), 8.63 (s, 1H), 8.50-8.43 (m, 2H), 7.75 (d, J = 9.2 Hz, 1H), 7.65-7.54 |
| | | (m, 3H), 7.02 (s, 1H), 6.97-6.95 (m, 1H), 4.93 (d, J = 6.0 Hz, 2H), 4.54- |
| | | 4.40 (m, 6H), 3.88-3.84 (m, 2H), 2.50 (s, 5H) ppm |
| 552 | 566 | 1H NMR (400 MHz, DMSO-d6) δ = 9.68-9.66 (m, 1H), 9.08 (s, 1H), 8.50 (d, J = |
| | | 1.6 Hz, 1H), 8.40 (d, J = 1.6 Hz, 1H), 8.32-8.25 (m, 2H), 7.77 (d, J = 1.2 Hz, 1H), |
| | | 7.50 (s, 1H), 7.21 (d, J = 1.4 Hz, 1H), 5.20 (s, 2H), 4.73 (br d, J = 5.6 Hz, 2H), 4.36- |
| | | 4.31 (m, 4H), 4.27-4.19 (m, 2H), 3.82-3.74 (m, 2H), 2.25 (s, 3H) ppm |
| 553 | 622.1 | 1H NMR (400 MHz, METHANOL-d4) δ = 9.31 (s, 1H), 8.69-8.64 (m, 2H), 8.62- |
| | | 8.56 (m, 1H), 8.50-8.46 (m, 1H), 8.34 (d, J = 2.0 Hz, 1H), 7.97-7.90 (m, 2H), 7.75- |
| | | 7.73 (m, 1H), 6.96 (d, J = 8.4 Hz, 1H), 5.69-5.64 (m, 1H), 4.92 (s, 2H), 4.42- |
| | | 4.27 (m, 3H), 3.91-3.82 (m, 2H), 3.81-3.74 (m, 2H), 3.65-3.58 (m, 1H), 2.59- |
| | | 2.53 (m, 2H), 1.70 (d, J = 6.4 Hz, 3H), 1.29 (d, J = 6.4 Hz, 6H) ppm |
| 554 | 564.2 | 1H NMR (400 MHz, DMSO-d6) δ = 9.65-9.62 (m, 1H), 9.08 (s, 1H), 8.39 (d, J = |
| | | 1.2 Hz, 1H), 8.33-8.24 (m, 2H), 8.18 (d, J = 1.6 Hz, 1H), 7.77 (d, J = 1.2 Hz, 1H), |
| | | 7.52 (s, 1H), 7.21 (d, J = 1.2 Hz, 1H), 5.12-5.02 (m, 1H), 5.00-4.91 (m, 1H), 4.73 |
| | | (d, J = 5.2 Hz, 2H), 4.37-4.25 (m, 5H), 4.02 (d, J = 13.2 Hz, 1H), 3.80-3.69 (m, |
| | | 1H), 2.25 (s, 3H), 1.18 (d, J = 7.2 Hz, 3H) ppm |
| 555 | 587.2 | 1H NMR (400 MHz, METHANOL-d4) δ = 8.90 (s, 1H), 8.61 (d, J = 1.6 Hz, 1H), 8.21- |
| | | 8.19 (m, 1H), 8.07 (d, J = 9.6 Hz, 1H), 7.64 (d, J = 8.0 Hz, 1H), 7.56 (s, 1H), 7.44 |
| | | (d, J = 9.2 Hz, 1H), 7.11-7.07 (m, 1H), 6.97 (d, J = 8.0 Hz, 1H), 6.33 (d, J = 8.0 |
| | | Hz, 1H), 5.33-5.30 (m, 1H), 5.08-5.02 (m, 4H), 4.82 (s, 2H), 4.73-4.70 (m, 2H), |
| | | 4.35-4.32 (m, 2H), 4.14-4.11 (m, 2H), 3.53-3.51 (m, 2H), 2.83-2.80 (m, 2H), |
| | | 2.03-1.96 (m, 2H) ppm |
| 556 | 530.2 | 1H NMR (400 MHz, DMSO-d6) δ = 9.59-9.57 (m, 1H), 9.07 (s, 1H), 8.54 (d, J = |
| | | 1.6 Hz, 1H), 8.30 (s, 1H), 8.27-8.25 (m, 1H), 8.19 (d, J = 9.2 Hz, 1H), 8.12-8.10 |
| | | (m, 1H), 7.96 (d, J = 9.2 Hz, 1H), 7.74 (d, J = 7.6 Hz, 1H), 7.60 (d, J = 7.2 |
| | | Hz, 1H), 7.51 (s, 1H), 7.05-7.03 (m, 1H), 4.98 (s, 2H), 4.74 (d, J = 5.6 Hz, 2H), |
| | | 4.50-4.43 (m, 1H), 4.26-4.22 (m, 2H), 3.72-3.67 (m, 2H), 3.55-3.49 (m, 1H), |
| | | 2.94-2.92 (m, 1H), 2.55 (br s, 1H), 2.16-2.06 (m, 1H), 1.06 (d, J = 6.8 Hz, 3H) ppm |
| 557 | 532.2 | 1H NMR (400 MHz, DMSO-d6) δ = 9.61-9.54 (m, 1H), 9.09 (s, 1H), 8.53 (d, J = |
| | | 1.8 Hz, 1H), 8.41-8.37 (m, 1H), 8.31-8.23 (m, 2H), 7.73 (d, J = 8.0 Hz, 1H), 7.49 |
| | | (s, 1H), 7.23 (d, J = 8.0 Hz, 1H), 6.91 (d, J = 8.0 Hz, 1H), 4.98 (s, 2H), 4.74 (br d, |
| | | J = 6.0 Hz, 2H), 4.34-4.27 (m, 4H), 4.25-4.20 (m, 2H), 3.70-3.65 (m, 2H), 2.36 (s, 3H) ppm |
| 558 | 540.2 | 1H NMR (400 MHz, DMSO-d6) δ = 9.60-9.51 (m, 1H), 9.05 (s, 1H), 8.51 (d, J = |
| | | 1.8 Hz, 1H), 8.38 (s, 1H), 8.28-8.21 (m, 2H), 7.85 (d, J = 1.2 Hz, 1H), 7.72 (d, J = |
| | | 7.8 Hz, 1H), 7.49-7.41 (m, 4H), 4.97 (s, 2H), 4.71 (d, J = 5.6 Hz, 2H), 4.24-4.20 |
| | | (m, 2H), 3.99-3.97 (m, 2H), 3.69-3.65 (m, 2H), 2.81-2.80 (m, 2H), 1.94-1.92 |
| | | (m, 2H) ppm |
| 559 | 602.3 | 1H NMR (400 MHz, DMSO-d6) δ = 9.55-9.54 (m, 1H), 9.38 (s, 1H), 8.67-8.60 (m, |
| | | 2H), 8.43 (s, 1H), 8.25-8.22 (m, 1H), 7.89 (d, J = 7.2 Hz, 1H), 7.79 (s, 1H), 7.73- |
| | | 7.71 (m, 1H), 7.30 (d, J = 8.4 Hz, 1 H), 7.02 (d, J = 6.4 Hz, 1 H) 4.79 (d, J = 4.2 Hz, |
| | | 2H), 4.31 (d, J = 11.6 Hz, 2H), 3.67-3.66 (m, 2H), 3.60-3.59 (m, 2H), 2.51-2.50 |
| | | (m, 2H), 2.24-2.21 (m, 2H), 1.31 (s, 6H), 1.20 (d, J = 6.0 Hz, 2H) ppm |
| 560 | 587.1 | 1H NMR (400 MHz, DMSO-d6) δ = 9.39 (s, 1H), 9.28-9.27 (m, 1H), 8.67-8.63 (m, |
| | | 2H), 8.34 (d, J = 2.4 Hz, 1H), 8.07-8.04 (m, 1H), 7.91 (d, J = 7.6 Hz, 1H), 7.76- |
| | | 7.70 (m, 2H), 7.03 (d, J = 8.4 Hz, 1H), 6.95 (d, J = 9.2 Hz, 1H), 4.75 (br d, J = 6.0 |
| | | Hz, 2H), 4.32 (br d, J = 11.2 Hz, 2H), 3.96-3.92 (m, 1H), 3.73-3.63 (m, 4H), 3.31 |
| | | (s, 3H), 2.54-2.52 (m, 2H), 1.27 (d, J = 6.8 Hz, 3H), 1.22 (d, J = 6.0 Hz, 6H) ppm |
| 561 | 574.3 | 1H NMR (400 MHz, DMSO-d6) δ = 9.57-9.56 (m, 1H), 9.06 (s, 1H), 8.53 (d, J = |
| | | 1.6 Hz, 1H), 8.32 (s, 1H), 8.25-8.23 (m, 1H), 8.18 (d, J = 9.2 Hz, 1H), 7.96 (d, J = |
| | | 9.2 Hz, 1H), 7.73 (d, J = 7.8 Hz, 1H), 7.60 (d, J = 7.6 Hz, 1H), 7.49 (s, |
| | | 1H), 7.04 (d, J = 7.6 Hz, 1H), 4.98 (s, 2H), 4.74 (d, J = 5.6 Hz, 2H), 4.25-4.19 (m, |
| | | 3H), 4.19-4.05 (m, 2H), 3.71-3.64 (m, 2H), 3.21 (s, 3H), 2.81-2.80 (m, 2H), 1.96- |
| | | 1.94 (m, 2H), 1.34 (d, J = 6.4 Hz, 3H) ppm |
| 562 | 574.3 | 1H NMR (400 MHz, DMSO-d6) δ = 9.57-9.55 (m, 1H), 9.06 (s, 1H), 8.53 (d, J = |
| | | 1.8 Hz, 1H), 8.33 (br s, 1H), 8.25-8.24 (m, 1H), 8.18 (d, J = 9.4 Hz, 1H), 7.96 (d, |
| | | J = 9.2 Hz, 1H), 7.73 (d, J = 7.9 Hz, 1H), 7.60 (d, J = 7.6 Hz, 1H), 7.49 (s, 1H), 7.04 |
| | | (d, J = 7.6 Hz, 1H), 4.98 (s, 2H), 4.74 (d, J = 5.8 Hz, 2H), 4.28-4.19 (m, 3H), 4.19- |
| | | 4.07 (m, 2H), 3.75-3.63 (m, 2H), 3.21 (s, 3H), 2.81-2.80 (m, 2H), 1.96-1.95 (m, |
| | | 2H), 1.34 (d, J = 6.6 Hz, 3H) ppm |
| 563 | 566.2 | 1H NMR (400 MHz, DMSO-d6) δ = 9.58-9.56 (m, 1H), 9.11 (s, 1H), 8.53 (d, J = |
| | | 1.8 Hz, 1H), 8.40 (s, 1H), 8.31-8.19 (m, 2H), 7.99 (d, J = 9.2 Hz, 1H), 7.75-7.73 |
| | | (m, 2H), 7.52 (s, 1H), 7.29 (d, J = 7.6 Hz, 1H), 7.00-6.64 (m, 1H), 4.98 |
| | | (s, 2H), 4.75 (d, J = 5.8 Hz, 2H), 4.26-4.20 (m, 2H), 4.20-4.11 (m, 2H), 3.74- |
| | | 3.64 (m, 2H), 2.88-2.86 (m, 2H), 2.04-1.91 (m, 2H) ppm |
| 564 | 574.3 | 1H NMR (400 MHz, DMSO-d6) δ = 9.57-9.56 (m, 1H), 9.06 (s, 1H), 8.53 (d, J = |
| | | 1.8 Hz, 1H), 8.38 (br s, 1H), 8.26-8.24 (m, 1H), 8.16 (d, J = 9.2 Hz, 1H), 8.00 (d, |
| | | J = 9.4 Hz, 1H), 7.73 (d, J = 8.0 Hz, 1H), 7.59 (d, J = 7.6 Hz, 1H), 7.48 (s, 1H), 7.06 |
| | | (d, J = 7.4 Hz, 1H), 4.98 (s, 2H), 4.73 (d, J = 5.8 Hz, 2H), 4.40 (s, 2H), 4.30-4.19 |
| | | (m, 2H), 4.18-4.09 (m, 2H), 3.73-3.64 (m, 2H), 3.53-3.51 (m, 2H), 2.81-2.80 |
| | | (m, 2H), 1.95-1.93 (m, 2H), 1.17-1.15 (m, 3H) ppm |
| 565 | 546.2 | 1H NMR (400 MHz, DMSO-d6) δ = 9.55-9.53 (m, 1H), 8.99 (s, 1H), 8.52 (d, J = |
| | | 2.0 Hz, 1H), 8.29-8.21 (m, 1H), 8.11 (d, J = 9.4 Hz, 1H), 7.97-7.66 (m, 3H), 7.43 |
| | | (s, 1H), 7.32 (d, J = 3.0 Hz, 1H), 4.97 (s, 2H), 4.71 (br d, J = 5.8 Hz, 2H), 4.27- |
| | | 4.17 (m, 2H), 4.16-4.06 (m, 2H), 3.81 (s, 3H), 3.72-3.64 (m, 2H), 2.80-2.78 (m, |
| | | 2H), 1.99-1.87 (m, 2H) ppm |
| 566 | 571.1 | 1H NMR (400 MHz, METHANOL-d4) δ = 9.12-9.03 (m, 1H), 8.65-8.59 (m, 1H), |
| | | 8.35-8.15 (m, 2H), 8.10-7.91 (m, 1H), 7.73-7.61 (m, 2H), 7.55-7.39 (m, 1H), |
| | | 6.18 (d, J = 8.0 Hz, 1H), 5.05 (s, 2H), 4.86 (s, 2H), 4.40-4.30 (m, 2H), 4.23-4.14 |
| | | (m, 2H), 4.14-3.91 (m, 4H), 3.58-3.49 (m, 2H), 2.77-2.74 (m, 2H), 2.37-2.18 |
| | | (m, 2H), 2.13-2.01 (m, 2H) ppm |
| 567 | 587.3 | 1H NMR (400 MHz, DMSO-d6) δ = 9.39 (s, 1H), 9.28-9.27 (m, 1H), 8.68-8.61 (m, |
| | | 2H), 8.35 (d, J = 2.0 Hz, 1H), 8.07-8.04 (m, 1H), 7.92 (d, J = 7.2 Hz, 1H), 7.77- |
| | | 7.70 (m, 2H), 7.03 (d, J = 8.4 Hz, 1H), 6.96 (d, J = 9.2 Hz, 1H), 4.77 (br d, J = 5.6 |
| | | Hz, 2H), 4.32 (br d, J = 11.4 Hz, 2H), 3.95-3.92 (m, 1H), 3.76-3.65 (m, 4H), 3.11 |
| | | (s, 3H), 2.54-2.53 (m, 2H), 1.27 (d, J = 6.8 Hz, 3H), 1.22 (d, J = 6.0 Hz, 6H) ppm |
| 568 | 546.2 | 1H NMR (400 MHz, DMSO-d6) δ = 9.58-9.56 (m, 1H), 9.08 (s, 1H), 8.53 (d, J = |
| | | 1.8 Hz, 1H), 8.32 (br s, 1H), 8.27-8.16 (m, 2H), 8.04 (d, J = 9.2 Hz, 1H), 7.73 (d, |
| | | J = 7.8 Hz, 1H), 7.59-7.41 (m, 2H), 6.46 (d, J = 8.0 Hz, 1H), 4.98 (s, 2H), 4.73 (d, |
| | | J = 5.8 Hz, 2H), 4.29-4.18 (m, 2H), 4.17-4.06 (m, 2H), 3.72-3.65 (m, 5H), 2.75- |
| | | 2.73 (m, 2H), 1.95-1.93 (m, 2H) ppm |
| 569 | 544.1 | 1H NMR (400 MHz, DMSO-d6) δ = 9.64-9.61 (m, 1H), 9.37 (s, 1H), 8.69-8.59 (m, |
| | | 2H), 8.54 (d, J = 2.0 Hz, 1H), 8.28-8.25 (m, 1H), 7.93 (d, J = 7.6 Hz, 1H), 7.79 (s, |
| | | 1H), 7.74 (d, J = 8.0 Hz, 1H), 7.54 (d, J = 7.6 Hz, 1H), 4.98 (s, 2H), 4.81 (d, J = 5.6 |
| | | Hz, 2H), 4.28-4.18 (m, 2H), 3.74-3.63 (m, 2H), 3.26-3.22 (m, 2H), 3.11 (s, 3H), |
| | | 2.83-2.75 (m, 2H), 1.90-1.78 (m, 2H), 1.76-1.65 (m, 2H) ppm |
| 570 | 584.2 | 1H NMR (400 MHz, DMSO-d6) δ = 9.67-9.52 (m, 1H), 9.12 (s, 1H), 8.57-8.50 (m, |
| | | 1H), 8.47-8.37 (m, 1H), 8.34-8.19 (m, 2H), 7.94 (d, J = 9.2 Hz, 1H), 7.86-7.77 |
| | | (m, 1H), 7.73 (d, J = 8.0 Hz, 1H), 7.54 (s, 1H), 7.45 (d, J = 7.6 Hz, 1H), 4.97 (s, 2H), |
| | | 4.75 (d, J = 6.4 Hz, 2H), 4.25-4.15 (m, 4H), 3.70-3.65 (m, 2H), 2.97-2.86 (m, |
| | | 2H), 2.05-1.96 (m, 2H) ppm |
| 57 | 580.2 | 1H NMR (400 MHz, METHANOL-d4) δ = 9.03 (s, 1H), 8.62 (d, J = 1.0 Hz, 1H), 8.55- |
| | | 8.44 (m, 2H), 8.19-8.17 (m, 2H), 8.00 (d, J = 9.2 Hz, 1H), 7.73-7.59 (m, 3H), |
| | | 7.29 (d, J = 7.4 Hz, 1H), 5.05 (s, 2H), 4.82-4.80 (m, 2H), 4.39-4.31 (m, 2H), 4.26- |
| | | 4.24 (m, 2H), 3.55-3.50 (m, 2H), 3.00-2.90 (m, 2H), 2.09-2.07(m, 2H), 1.89- |
| | | 1.86 (m, 3H) ppm |
| 572 | 533.2 | 1H NMR (400 MHz, DMSO-d6) δ = 9.57-9.56 (m, 1H), 9.03 (s, 1H), 8.51 (d, J = |
| | | 1.6 Hz, 1H), 8.35 (d, J = 2.0 Hz, 1H), 8.29-8.14 (m, 2H), 7.73 (d, J = 7.6 Hz, 1H), |
| | | 7.47 (d, J = 9.2 Hz, 1H), 7.41 (s, 1H), 7.31-7.17 (m, 2H), 6.89-6.86(m, 1H), 4.97 |
| | | (s, 2H), 4.71 (d, J = 6.0 Hz, 2H), 4.26-4.18 (m, 2H), 3.99-3.97 (m, 2H), 3.72- |
| | | 3.61 (m, 2H), 2.73-2.71 (m, 2H), 1.95-1.89 (m, 2H) ppm |
| 573 | 560.2 | 1H NMR (400 MHz, DMSO-d6) δ = 9.57-9.54 (m, 1H), 9.04 (s, 1H), 8.53 (d, J = |
| | | 1.6 Hz, 1H), 8.26-8.23 (m, 1H), 8.15 (d, J = 9.2 Hz, 1H), 8.04 (d, J = 6.0 Hz, 1H), |
| | | 7.90 (d, J = 9.2 Hz, 1H), 7.73 (d, J = 7.6 Hz, 1H), 7.47 (s, 1H), 6.79 (d, J = 5.6 Hz, |
| | | 1H), 4.98 (s, 2H), 4.72 (d, J = 6.0 Hz, 2H), 4.23-4.21 (m, 2H), 4.18-4.11 (m, 4H), |
| | | 3.69-3.66 (m, 2H), 2.68-2.65 (m, 2H), 1.94-1.88 (m, 2H), 1.38-1.34 (m, 3H) ppm |
| 574 | 553.2 | 1H NMR (400 MHz, DMSO-d6) δ = 9.67-9.65 (m, 1H), 9.27 (s, 1H), 8.79 (d, J = |
| | | 4.0 Hz, 1H), 8.63 (d, J = 8.8 Hz, 1H), 8.52 (s, 1H), 8.39 (s, 2H), 8.26 (d, J = 8.0 Hz, |
| | | 1H), 8.05 (d, J = 9.2 Hz, 1H), 7.74 (d, J = 7.6 Hz, 1H), 7.59 (s, 1H), 4.98 (s, 2H), |
| | | 4.75 (d, J = 6.4 Hz, 2H), 4.23-4.21 (m, 2H), 3.76-3.74 (m, 4H), 3.69-3.66 (m, 2H), |
| | | 3.31 = 3.29 (m, 4H) ppm |
| 575 | 566.2 | 1H NMR (400 MHz, DMSO-d6) δ = 9.71-9.68 (m, 1H), 9.39 (s, 1H), 8.71-8.65 (m, |
| | | 1H), 8.63-8.57 (m, 2H), 8.32-8.30 (m, 1H), 7.94 (d, J = 8.4 Hz, 1H), 7.86-7.77 |
| | | (m, 2H), 7.71-7.67 (m, 1H), 7.04 (d, J = 8.4 Hz, 1H), 4.81 (d, J = 6.0 Hz, 2H), 4.75- |
| | | 4.56 (m, 1H), 4.50-4.37 (m, 1H), 4.32-4.28 (m, 1H), 3.43 (s, 3H), 3.24-3.17 (m, |
| | | 1H), 3.01-2.89 (m, 1H), 2.64-2.58 (m, 1H), 1.91-1.87 (m, 1H), 1.50-1.34 (m, |
| | | 2H), 1.01 (d, J = 6.8 Hz, 3H) ppm |
| 576 | 560.2 | 1H NMR (400 MHz, DMSO-d6) δ = 9.62-9.54 (m, 1H), 9.08 (s, 1H), 8.52 (d, J = |
| | | 1.6 Hz, 1H), 8.39 (s, 1H), 8.28-8.17 (m, 2H), 7.99 (d, J = 9.2 Hz, 1H), 7.73 (d, J = |
| | | 7.6 Hz, 1H), 7.54-7.46 (m, 2H), 6.47-6.40 (m, 1H), 4.99-4.95 (m, 2H), 4.73 (d, |
| | | J = 5.6 Hz, 2H), 4.25-4.20 (m, 2H), 4.17-4.07 (m, 4H), 3.70-3.66 (m, 2H), 2.75- |
| | | 2.72 (m, 2H), 1.99-1.90 (m, 2H), 1.26-1.22 (m, 3H) ppm |
| 577 | 585.3 | 1H NMR (400 MHz, DMSO-d6) δ = 9.73-9.70 (m, 1H), 9.40 (s, 1H), 9.14 (d, J = |
| | | 2.0 Hz, 1H), 8.70-8.61 (m, 3H), 8.41 (br s, 1H), 7.91 (d, J = 7.2 Hz, 1H), 7.83 (s, |
| | | 1H), 7.77-7.73 (m, 1H), 7.04 (d, J = 8.4 Hz, 1H), 4.83 (br d, J = 5.6 Hz, 2H), 4.32 |
| | | (br d, J = 11.6 Hz, 2H), 3.83-3.75 (m, 2H), 3.72-3.66 (m, 2H), 2.58-2.55 (m, 2H), |
| | | 2.48-2.41 (m, 2H), 1.98-1.88 (m, 1H), 1.73-1.66 (m, 1H), 1.54-1.48 (m, 1H), |
| | | 1.22 (d, J = 6.0 Hz, 6H), 0.75-0.64 (m, 1H) ppm |
| 578 | 602.2 | 1H NMR (400 MHz, DMSO-d6) δ = 9.61-9.59 (m, 1H), 9.40 (s, 1H), 8.67-8.63 |
| | | (m, 3H), 8.52 (s, 1H), 8.31-8.29 (m, 1H), 7.91 (d, J = 7.2 Hz, 1H), 7.83-7.81 (m, |
| | | 2H), 7.74-7.73 (m, 1H), 7.02 (d, J = 8.4 Hz, 1H), 4.98 (s, 2H), 4.81 (d, J = 6.4 Hz, |
| | | 2H), 4.31 (d, J = 12 Hz, 2 H), 3.69-3.65 (m, 2H), 3.50-3.48 (m, 1H), 3.31 (s, 3H), |
| | | 2.51-2.50 (m, 2H), 1.21 (d, J = 6.4 Hz, 6H), 0.64-0.62 (m, 2H), 0.54-0.49 (m, 2H) ppm |
| 579 | 585.3 | 1H NMR (400 MHz, DMSO-d6) δ = 9.73-9.71 (m, 1H), 9.40 (s, 1H), 9.14 (d, J = |
| | | 2.0 Hz, 1H), 8.70-8.61 (m, 3H), 8.42 (br s, 1H), 7.91 (d, J = 7.6 Hz, 1H), 7.83 (s, |
| | | 1H), 7.77-7.73 (m, 1H), 7.04 (d, J = 8.4 Hz, 1H), 4.83 (br d, J = 5.6 Hz, 2H), 4.32 |
| | | (br d, J = 11.2 Hz, 2H), 3.81-3.75 (m, 2H), 3.73-3.66 (m, 2H), 2.65-2.56 (m, 2H), |
| | | 2.43 (br s, 2H), 1.99-1.87 (m, 1H), 1.74-1.66 (m, 1H), 1.52-1.50 (m, 1H), 1.22 |
| | | (d, J = 6.0 Hz, 6H), 0.75-0.64 (m, 1H) ppm |
| 580 | 602.3 | 1H NMR (400 MHz, DMSO-d6) δ = 9.57-9.56 (m, 1H), 9.39 (s, 1H), 8.70-8.59 (m, |
| | | 2H), 8.48 (s, 1H), 8.46 (d, J = 2.4 Hz, 1H), 8.27 (d, J = 2.4 Hz, 1H), 7.90 (d, J = 7.6 |
| | | Hz, 1H), 7.80-7.70 (m, 2H), 7.40 (d, J = 8.4 Hz, 1H), 7.03 (d, J = 8.4 Hz, 1H), 4.80 |
| | | (d, J = 5.6 Hz, 2H), 4.31 (d, J = 11.2 Hz, 2H), 3.90 (d, J = 3.6 Hz, 1H), 3.68-3.67 |
| | | (m, 2H), 3.64-3.58 (m, 2H), 2.52 (s, 2H), 2.31-2.22 (m, 1H), 2.19-2.09 (m, 1H), |
| | | 1.83-1.73 (m, 1H), 1.73-1.63 (m, 1H), 1.21 (d, J = 6.4 Hz, 6H), 1.10-1.06 (m, 3H) ppm |
| 581 | 624.3 | 1H NMR (400 MHz, DMSO-d6) δ = 9.63-9.61 (m, 1H), 9.40 (s, 1H), 8.69-8.60 (m, |
| | | 2H), 8.48 (d, J = 2.0 Hz, 1H), 8.32 (d, J = 2.0 Hz, 1H), 7.89 (d, J = 7.6 Hz, 1H), 7.81- |
| | | 7.71 (m, 2H), 7.45 (d, J = 8.4 Hz, 1H), 7.03 (d, J = 8.4 Hz, 1H), 6.63-6.18 (m, |
| | | 1H), 4.81 (d, J = 5.6 Hz, 2H), 4.43-4.26 (m, 3H), 3.81-3.64 (m, 4H), 2.53-2.52 |
| | | (m, 2H), 2.42-2.35 (m, 1H), 2.28-2.20 (m, 1H), 1.21 (d, J = 6.0 Hz, 6H) ppm |
| 582 | 624.4 | 1H NMR (400 MHz, DMSO-d6) δ = 9.70-9.52 (m, 1H), 9.40 (s, 1H), 8.73-8.58 (m, |
| | | 2H), 8.48 (d, J = 2.2 Hz, 1H), 8.32-8.30 (m, 1H), 7.89 (d, J = 7.6 Hz, 1H), 7.83- |
| | | 7.70 (m, 2H), 7.45 (d, J = 8.4 Hz, 1H), 7.03 (d, J = 8.4 Hz, 1H), 6.62-6.16 (m, 1H), |
| | | 4.81 (br d, J = 5.6 Hz, 2H), 4.43-4.25 (m, 3H), 3.82-3.62 (m, 4H), 2.52 (br s, 2H), |
| | | 2.38-2.36 (m, 1H), 2.29-2.20 (m, 1H), 1.21 (d, J = 6.2 Hz, 6H) ppm |
| 583 | 586.2 | 1H NMR (400 MHz, DMSO-d6) δ = 9.69-9.57 (m, 1H), 9.16 (s, 1H), 8.53 (d, J = |
| | | 1.6 Hz, 1H), 8.44 (br s, 1H), 8.40 (d, J = 9.2 Hz, 1H), 8.31-8.23 (m, 2H), 7.73 (d, |
| | | J = 8.0 Hz, 1H), 7.55 (s, 1H), 7.51 (s, 2H), 4.98 (s, 2H), 4.76 (d, J = 5.6 Hz, 2H), 4.47- |
| | | 4.36 (m, 4H), 4.27-4.20 (m, 2H), 3.70-3.65 (m, 2H) ppm |
| 584 | 602.3 | 1H NMR (400 MHz, DMSO-d6) δ = 9.58-9.56 (m, 1H), 9.40 (s, 1H), 8.72-8.55 (m, |
| | | 2H), 8.48 (br s, 1H), 8.46 (d, J = 2.4 Hz, 1H), 8.27-8.26 (m, 1H), 7.90 (d, J = 7.6 |
| | | Hz, 1H), 7.81-7.70 (m, 2H), 7.40 (d, J = 8.4 Hz, 1H), 7.03 (d, J = 8.6 Hz, 1H), 4.80 |
| | | (br d, J = 5.8 Hz, 2H), 4.31 (br d, J = 11.4 Hz, 2H), 3.94-3.83 (m, 1H), 3.74-3.58 |
| | | (m, 4H), 2.52 (br s, 2H), 2.27-2.25 (m, 1H), 2.19-2.06 (m, 1H), 1.87-1.62 (m, |
| | | 2H), 1.21 (d, J = 6.2 Hz, 6H), 1.08-1.06 (m, 3H) ppm |
| 585 | 584.3 | 1H NMR (400 MHz, DMSO-d6) δ = 9.66-9.63 (m, 1H), 9.47 (s, 1H), 8.75 (d, J = |
| | | 8.4 Hz, 1H), 8.55 (d, J = 1.6 Hz, 1H), 8.45 (d, J = 4.8 Hz, 1H), 8.38 (d, J = 8.8 Hz, |
| | | 1H), 8.27-8.26 (m, 1H), 7.93 (s, 1H), 7.82-7.80 (m, 2H), 7.74 (d, J = 7.6 Hz, 1H), |
| | | 7.21 (d, J = 3.6 Hz, 1H), 4.99 (s, 2H), 4.86 (br d, J = 5.6 Hz, 2H), 4.54 (s, 2H), 4.27- |
| | | 4.21 (m, 2H), 3.72-3.66 (m, 2H), 3.29 (s, 3H), 0.90-0.85 (m, 2H), 0.81-0.74 (m, 2H) ppm |
| 586 | 589.1 | 1H NMR (400 MHz, METHANOL-d4) δ = 8.92 (s, 1H), 8.63 (d, J = 1.6 Hz, 1H), 8.22 |
| | | 8.20 (m, 1H), 8.09 (d, J = 9.4 Hz, 1H), 7.65 (d, J = 7.8 Hz, 1H), 7.59 (s, 1H), 7.51- |
| | | (d, J = 9.2 Hz, 1H), 7.16 (d, J = 8.4 Hz, 1H), 6.94 (d, J = 2.4 Hz, 1H), 6.74-6.72 |
| | | (m, 1H), 5.06 (s, 2H), 4.84 (br s, 2H), 4.41-4.33 (m, 2H), 4.12-4.04 (m, 4H), 3.78- |
| | | 3.70 (m, 2H), 3.56-3.52 (m, 2H), 3.41 (s, 3H), 2.73-2.71 (m, 2H), 2.01-2.00 (m, 2H) ppm |
| 587 | 608.1 | 1H NMR (400 MHz, DMSO-d6) δ = 9.63-9.61 (m, 1H), 9.40 (s, 1H), 8.70-8.60 (m, |
| | | 2H), 8.52 (d, J = 1.8 Hz, 1H), 8.45 (s, 1H), 8.28-8.26 (m, 1H), 7.91 (d, J = 7.4 Hz, |
| | | 1H), 7.84-7.78 (m, 2H), 7.74-7.72 (m, 1H), 7.03 (d, J = 8.4 Hz, 1H), 4.82 (d, J = |
| | | 5.8 Hz, 2H), 4.31 (br d, J = 11.6 Hz, 2H), 3.97-3.81 (m, 2H), 3.72-3.64 (m, 2H), |
| | | 3.64-3.58 (m, 2H), 2.73-2.63 (m, 2H), 2.52 (br d, J = 2.0 Hz, 2H), 1.21 (d, J = 6.4 |
| | | Hz, 6H) ppm |
| 588 | 550.2 | 1H NMR (400 MHz, DMSO-d6) δ = 9.59-9.56 (m, 1H), 9.09 (s, 1H), 8.53 (d, J = |
| | | 2.0 Hz, 1H), 8.34 (s, 1H), 8.26-8.24 (m, 1H), 8.21 (d, J = 8.8 Hz, 1H), 8.14 (d, J = |
| | | 2.4 Hz, 1H), 7.89 (d, J = 9.2 Hz, 1H), 7.77-7.71 (m, 2H), 7.50 (s, 1H), 4.98 (s, 2H), |
| | | 4.74 (d, J = 5.6 Hz, 2H), 4.23-4.21 (m, 2H), 4.16-4.08 (m, 2H), 3.73-3.66 (m, |
| | | 2H), 2.85-2.82 (m, 2H), 2.01-1.92 (m, 2H) ppm |
| 589 | 586.3 | 1H NMR (400 MHz, CHLOROFORM-d) δ = 8.99 (s, 1H), 8.55 (d, J = 2.0 Hz, 1H), |
| | | 8.20-8.17 (m, 1H), 8.08-8.01 (m, 1H), 7.97-7.90 (m, 1H), 7.74 (br s, 1H), 7.68 |
| | | (s, 1H), 7.49 (d, J = 7.6 Hz, 1H), 7.38 (d, J = 8.0 Hz, 1H), 6.45 (d, J = 8.0 Hz, 1H), |
| | | 5.04 (s, 2H), 4.92 (d, J = 5.2 Hz, 2H), 4.42-4.36 (m, 2H), 4.28-4.25 (m, 2H), 3.97 |
| | | (d, J = 6.8 Hz, 2H), 3.46-3.36 (m, 2H), 2.81-2.78 (m, 2H), 2.10-2.03 (m, 2H), |
| | | 1.25-1.17 (m, 1H), 0.61-0.53 (m, 2H), 0.31-0.24 (m, 2H) ppm |
| 590 | 574.3 | 1H NMR (400 MHz, DMSO-d6) δ = 9.64-9.63 (m, 1H), 9.27 (br s, 1H), 8.59-8.43 |
| | | (m, 1H), 8.33-8.21 (m, 2H), 8.14 (br d, J = 9.0 Hz, 1H), 7.78-7.68 (m, 2H), 7.56 |
| | | (s, 1H), 4.98 (s, 2H), 4.82 (br d, J = 4.6 Hz, 2H), 4.41 (s, 2H), 4.30-4.14 (m, 4H), |
| | | 3.70-3.66 (m, 2H), 3.34 (br s, 3H), 2.81-2.80 (m, 2H), 2.38 (s, 3H), 2.01-1.89 |
| | | (m, 2H) ppm |
| 591 | 534.2 | 1H NMR (400 MHz, CHLOROFORM-d) δ = 8.99 (s, 1H), 8.55 (d, J = 1.6 Hz, 1H), |
| | | 8.20-8.17 (m, 1H), 8.05 (d, J = 2.8 Hz, 1H), 8.01-7.93 (m, 2H), 7.75 (s, 1H), 7.69 |
| | | (s, 1H), 7.49 (d, J = 8.0 Hz, 1H), 5.04 (s, 2H), 4.93 (d, J = 5.2 Hz, 2H), 4.43-4.34 |
| | | (m, 2H), 4.33-4.27 (m, 2H), 3.45-3.38 (m, 2H), 2.91-2.88 (m, 2H), 2.13-2.06 |
| | | (m, 2H) ppm |
| 592 | 559.3 | 1H NMR (400 MHz, DMSO-d6) δ = 9.67-9.64 (m, 1H), 9.47 (s, 1H), 8.80 (d, J = |
| | | 8.8 Hz, 1H), 8.66 (d, J = 8.8 Hz, 1H), 8.53 (d, J = 1.6 Hz, 1H), 8.29-8.24 (m, 2H), |
| | | 8.14-7.69 (m, 3H), 7.34-7.31 (m, 1H), 4.97 (s, 2H), 4.84 (d, J = 5.6 Hz, 2H), 4.31- |
| | | 4.27 (m, 1H), 4.04-3.99 (m, 1H), 3.72-3.58 (m, 1H), 1.17 (d, J = 7.2 Hz, 3H) ppm |
| 593 | 545.3 | 1H NMR (400 MHz, DMSO-d6) δ = 9.68-9.66 (m, 1H), 9.25 (s, 1H), 8.52 (d, J = |
| | | 2.0 Hz, 1H), 8.33-8.24 (m, 2H), 8.20-8.11 (m, 1H), 7.74 (d, J = 8.0 Hz, 1H), 7.66 |
| | | (s, 1H), 7.09 (d, J = 8.0 Hz, 1H), 6.95 (d, J = 8.4 Hz, 1H), 4.98 (s, 2H), 4.81 (d, J = |
| | | 5.6 Hz, 2H), 4.38-4.30 (m, 2H), 4.24-4.21 (m, 2H), 3.71-3.65 (m, 2H), 3.38- |
| | | 3.36 (m, 2H), 2.91 (s, 3H), 2.32 (s, 3H) ppm |
| 594 | 568.2 | 1H NMR (400 MHz, DMSO-d6) δ = 9.70-9.60 (m, 1H), 9.33-9.24 (m, 1H), 8.52 |
| | | (d, J = 1.7 Hz, 1H), 8.47-8.39 (m, 2H), 8.26 (br d, J = 8.0 Hz, 1H), 7.74 (d, J = 8.0 |
| | | Hz, 1H), 7.70-7.63 (m, 1H), 7.51 (br d, J = 8.2 Hz, 1H), 7.39 (br d, J = 8.0 Hz, 1H), |
| | | 7.06-6.72 (m, 1H), 4.98 (s, 2H), 4.80 (br s, 2H), 4.43 (s, 4H), 4.23-4.21 (m, 2H), |
| | | 3.70-3.65 (m, 2H) ppm |
| 595 | 559.3 | 1H NMR (400 MHz, METHANOL-d4) δ = 9.18 (s, 1H), 8.64 (d, J = 1.6 Hz, 1H), 8.50- |
| | | 8.38 (m, 1H), 8.29-8.17 (m, 1H), 8.08-7.91 (m, 1H), 7.75-7.55 (m, 3H), 6.57 (br |
| | | d, J = 8.8 Hz, 1H), 5.06 (s, 2H), 4.89 (s, 2H), 4.40-4.31 (m, 2H), 4.28-4.19 (m, |
| | | 2H), 3.61-3.51 (m, 2H), 3.21-3.08 (m, 6H), 2.86-2.75 (m, 2H), 2.22-2.07 (m, 2H) ppm |
| 596 | 573.2 | 1H NMR (400 MHz, DMSO-d6) δ = 9.76-9.73 (m, 1H), 9.40 (s, 1H), 9.26 (d, J = |
| | | 2.0 Hz, 1H), 8.81 (d, J = 2.0 Hz, 1H), 8.68-8.61 (m, 2H), 7.92-7.90 (m, 1H), 7.86 |
| | | (s, 1H), 7.76-7.73 (m, 1H), 7.03 (d, J = 8.4 Hz, 1H), 4.82 (d, J = 5.6 Hz, 2H), 4.31 |
| | | (d, J = 11.2 Hz, 2H), 3.69-3.65 (m, 3H), 3.24-3.22 (m, 2H), 2.52-2.50 (m, 2H), |
| | | 2.38-2.35 (m, 1H), 2.22-2.19 (m, 1H), 1.37 (d, J = 6.8 Hz, 3H), 1.21 (d, J = 6.4 |
| | | Hz, 6H) ppm |
| 597 | 573.2 | 1H NMR (400 MHz, DMSO-d6) δ = 9.76-9.73 (m, 1H), 9.40 (s, 1H), 9.26 (d, J = |
| | | 2.0 Hz, 1H), 8.81 (d, J = 2.0 Hz, 1H), 8.67-8.61 (m, 2H), 7.93-7.90 (m, 1H), 7.86 |
| | | (s, 1H), 7.76-7.73 (m, 1H), 7.02 (d, J = 8.4 Hz, 1H), 4.83 (d, J = 5.6 Hz, 2H), 4.31 |
| | | (d, J = 11.2 Hz, 2H), 3.69-3.65 (m, 3H), 3.24-3.22 (m, 2H), 2.52-2.50 (m, 2H), |
| | | 2.37-2.35 (m, 1H), 2.22-2.19 (m, 1H), 1.37 (d, J = 6.8 Hz, 3H), 1.21 (d, J = 6.4 |
| | | Hz, 6H) ppm |
| 598 | 586.3 | 1H NMR (400 MHz, DMSO-d6) δ = 9.60-9.57 (m, 1H), 9.06 (s, 1H), 8.53 (s, 1H), |
| | | 8.26-8.24(m, 1H), 8.16 (d, J = 9.2 Hz, 1H), 8.01 (d, J = 9.2 Hz, 1H), 7.73 (d, J = |
| | | 8.0 Hz, 1H), 7.58 (d, J = 7.6 Hz, 1H), 7.48 (s, 1H), 7.03 (d, J = 7.6 Hz, 1H), 4.97 (s, |
| | | 2H), 4.73 (d, J = 5.6 Hz, 2H), 4.44 (s, 2H), 4.23-4.21 (m, 2H), 4.15-4.12 (m, 2H), |
| | | 3.69-3.67 (m, 2H), 3.41-3.39 (m, 1H), 2.81-2.78 (m, 2H), 1.96-1.93 (m, 2H), |
| | | 0.57-0.53 (m, 2H), 0.46-0.45 (m, 2H) ppm |
| 599 | 594.2 | 1H NMR (400 MHz, DMSO-d6) δ = 9.58-9.56 (m, 1H), 9.09 (s, 1H), 8.53 (d, J = 1.6 |
| | | Hz, 1H), 8.28-8.19 (m, 2H), 8.00 (d, J = 9.2 Hz, 1H), 7.77-7.70 (m, 2H), 7.50 (s, |
| | | 1H), 4.98 (s, 2H), 4.74 (d, J = 5.8 Hz, 2H), 4.44 (s, 2H), 4.26-4.19 (m, 2H), 4.17- |
| | | 4.09 (m, 2H), 3.73-3.62 (m, 2H), 3.30 (s, 3H), 2.84-2.82 (m, 2H), 2.01-1.87 (m, 2H) ppm |
| 600 | 542.2 | 1H NMR (400 MHz, DMSO-d6) δ = 9.64-9.52 (m, 1H), 9.07 (s, 1H), 8.53 (d, J = |
| | | 1.6 Hz, 1H), 8.47 (s, 1H), 8.25-8.23 (m, 1H), 8.19 (d, J = 9.0 Hz, 1H), 8.05-8.02 |
| | | (m, 1H), 7.92 (d, J = 9.2 Hz, 1H), 7.73 (d, J = 8.0 Hz, 1H), 7.49 (s, 1H), 7.24-7.22 |
| | | (m, 1H), 7.01-6.99 (m, 1H), 4.98 (s, 2H), 4.73 (br d, J = 6.0 Hz, 2H), 4.32-4.15 |
| | | (m, 4H), 3.73-3.62 (m, 2H), 1.91-1.81 (m, 2H), 1.13-1.01 (m, 2H), 1.00-0.89 (m, 2H) ppm |
| 601 | 596.2 | 1H NMR (400 MHz, DMSO-d6) δ = 9.59-9.56 (m, 1H), 9.11 (s, 1H), 8.52 (d, J = |
| | | 1.6 Hz, 1H), 8.44 (s, 1H), 8.27-8.20 (m, 2H), 7.98 (d, J = 9.2 Hz, 1H), 7.73 (d, J = |
| | | 7.6 Hz, 1H), 7.57 (d, J = 8.0 Hz, 1H), 7.50 (s, 1H), 6.53 (d, J = 8.0 Hz, 1H), 6.48- |
| | | 6.14 (m, 1H), 4.98 (s, 2H), 4.73 (d, J = 5.6 Hz, 2H), 4.42-4.33 (m, 2H), 4.26-4.18 |
| | | (m, 2H), 4.14-4.07 (m, 2H), 3.71-3.64 (m, 2H), 2.78-2.75 (m, 2H), 2.01-1.90 (m, 2H) ppm |
| 602 | 560.3 | 1H NMR (400 MHz, DMSO-d6) δ = 9.63-9.51 (m, 1H), 9.05-8.94 (m, 1H), 8.53 (s, |
| | | 1H), 8.43 (br s, 1H), 8.26 (br d, J = 7.2 Hz, 1H), 8.10 (br d, J = 9.2 Hz, 1H), 7.86 (d, |
| | | J = 9.0 Hz, 1H), 7.73 (br d, J = 7.8 Hz, 1H), 7.42 (s, 1H), 7.28 (s, 1H), 4.98 (s, 2H), |
| | | 4.82-4.59 (m, 2H), 4.30-4.17 (m, 2H), 4.11-4.10 (m, 2H), 3.81 (s, 3H), 3.69 (br |
| | | d, J = 3.8 Hz, 2H), 2.79-2.77 (m, 2H), 2.28 (s, 3H), 1.96-1.88 (m, 2H) ppm |
| 603 | 534.3 | 1H NMR (400 MHz, DMSO-d6) δ = 9.58-9.56 (m, 1H), 9.12 (s, 1H), 8.53 (d, J = |
| | | 1.6 Hz, 1H), 8.43 (br s, 1H), 8.31-8.23 (m, 2H), 7.96 (d, J = 9.2 Hz, 1H), 7.79- |
| | | 7.71 (m, 2H), 7.52 (s, 1H), 6.72-6.70 (m, 1H), 4.98 (s, 2H), 4.75 (d, J = 5.6 Hz, |
| | | 2H), 4.26-4.19 (m, 2H), 4.15-4.09 (m, 2H), 3.70-3.65 (m, 2H), 2.82 2.80 (m, |
| | | 2H), 2.01-1.91 (m, 2H) ppm |
| 604 | 572.1 | 1H NMR (400 MHz, DMSO-d6) δ = 9.65-9.63 (m, 1H), 9.39 (s, 1H), 8.69-8.59 (m, |
| | | 2H), 8.55 (d, J = 1.6 Hz, 1H), 8.28-8..25 (m, 1H), 7.89 (d, J = 7.2 Hz, 1H), 7.81 (s, |
| | | 1H), 7.77-7.68 (m, 2H), 6.57 (d, J = 8.4 Hz, 1H), 4.99 (s, 2H), 4.82 (br d, J = 5.6 |
| | | Hz, 2H), 4.72-4.72 (m, 2H), 4.36-4.33 (m, 2H), 4.26-4.17 (m, 4H), 3.82-3.78 |
| | | (m, 2H), 3.74-3.67 (m, 2H), 3.31-3.28 (m, 1H), |
| | | 3.16-3.08 (m, 1H) ppm |
| 605 | 559.2 | 1H NMR (400 MHz, DMSO-d6) δ = 9.67-9.48 (m, 1H), 8.98 (s, 1H), 8.52 (d, J = |
| | | 1.6 Hz, 1H), 8.44 (s, 1H), 8.28-8.21 (m, 1H), 8.15 (d, J = 8.8 Hz, 1H), 7.73 (d, J = |
| | | 8.0 Hz, 1H), 7.42-7.35 (m, 2H), 7.30 (s, 1H), 7.22 (d, J = 7.6 Hz, 1H), 7.05-6.96 |
| | | (m, 1H), 4.98 (s, 2H), 4.70 (d, J = 5.6 Hz, 2H), 4.35 (s, 2H), 4.26-4.19 (m, 2H), |
| | | 4.04-3.95 (m, 2H), 3.73-3.65 (m, 2H), 3.25 (s, 3H), 2.76-2.69 (m, 2H), 1.96- |
| | | 1.87 (m, 2H) ppm |
| 606 | 557.3 | 1H NMR (400 MHz, DMSO-d6) δ = 9.58-9.55 (m, 1H), 9.03 (s, 1H), 8.52 (d, J = |
| | | 1.6 Hz, 1H), 8.45-8.42 (m, 1H), 8.29-8.22 (m, 2H), 7.73 (d, J = 8.0 Hz, 1H), 7.52 |
| | | (d, J = 9.2 Hz, 1H), 7.39 (s, 1H), 7.19 (d, J = 2.0 Hz, 1H), 6.88-6.80 (m, 1H), 6.80- |
| | | 6.72 (m, 1H), 4.97 (s, 2H), 4.71 (d, J = 6.0 Hz, 2H), 4.25-4.17 (m, 6H), 3.69-3.66 |
| | | (m, 2H), 1.88-1.81 (m, 1H), 0.88-0.81 (m, 2H), 0.59-0.53 (m, 2H) ppm |
| 607 | 574.3 | 1H NMR (400 MHz, DMSO-d6) δ = 9.58 (s, 1H), 9.06 (s, 1H), 8.53 (d, J = 1.6 Hz, |
| | | 1H), 8.39 (s, 1H), 8.26 (d, J = 7.6 Hz, 1H), 8.17 (d, J = 9.2 Hz, 1H), 8.06 (d, J = 2.0 |
| | | Hz, 1H), 7.95 (d, J = 9.2 Hz, 1H), 7.73 (d, J = 8.0 Hz, 1H), 7.55 (d, J = 2.0 Hz, 1H), |
| | | 7.48 (s, 1H), 4.98 (s, 2H), 4.73 (d, J = 6.0 Hz, 2H), 4.34-4.32 (m, 1H), 4.26-4.19 |
| | | (m, 2H), 4.17-4.10 (m, 2H), 3.72-3.65 (m, 2H), 3.15 (s, 3H), 2.86-2.83 (m, 2H), |
| | | 2.01-1.92 (m, 2H), 1.38 (d, J = 6.4 Hz, 3H) ppm |
| 608 | 574.3 | 1H NMR (400 MHz, DMSO-d6) δ = 9.60-9.57 (m, 1H), 9.06 (s, 1H), 8.53 (d, J = |
| | | 1.6 Hz, 1H), 8.40 (s, 1H), 8.26 (d, J = 7.6 Hz, 1H), 8.17 (d, J = 9.2 Hz, 1H), 8.06 (d, |
| | | J = 2.0 Hz, 1H), 7.95 (d, J = 9.2 Hz, 1H), 7.73 (d, J = 8.0 Hz, 1H), 7.55 (d, J = 2.0 |
| | | Hz, 1H), 7.48 (s, 1H), 4.98 (s, 2H), 4.73 (d, J = 5.6 Hz, 2H), 4.34-4.32 (m, 1H), |
| | | 4.28-4.20 (m, 2H), 4.18-4.10 (m, 2H), 3.73-3.65 (m, 2H), 3.15 (s, 3H), 2.86- |
| | | 2.84 (m, 2H), 2.00-1.96 (m, 2H), 1.38 (d, J = 6.4 Hz, 3H) ppm |
| 609 | 605.3 | 1H NMR (400 MHz, DMSO-d6) δ = 9.67-9.65 (m, 1H), 9.43 (s, 1H), 8.65 (d, J = |
| | | 8.8 Hz, 1H), 8.51 (d, J = 1.6 Hz, 1H), 8.39 (br s, 1H), 8.28-8.26 (m, 1H), 8.02- |
| | | 8.00 (m, 1H), 7.82 (s, 1H), 7.73 (d, J = 7.8 Hz, 1H), 7.54-7.48 (m, 1H), 7.29-7.24 |
| | | (m, 1H), 7.22-7.16 (m, 1H), 4.96 (s, 2H), 4.82 (br d, J = 5.4 Hz, 2H), 4.28-4.26 |
| | | (m, 1H), 4.01-4.00 (m, 1H), 3.82-3.74 (m, 2H), 3.68-3.61 (m, 1H), 3.31 (br d, J = |
| | | 11.2 Hz, 2H), 2.43 2.41 (m, 2H), 1.17-1.12 (m, 9H) ppm |
| 610 | 606.3 | 1H NMR (400 MHz, DMSO-d6) δ = 9.66-9.64 (m, 1H), 9.47 (s, 1H), 8.73 (d, J = |
| | | 8.6 Hz, 1H), 8.51 (d, J = 1.2 Hz, 1H), 8.41 (br s, 1H), 8.27-8.25 (m, 1H), 8.14 (d, |
| | | J = 5.0 Hz, 1H), 8.08-8.06 (m, 1H), 7.86 (s, 1H), 7.73 (d, J = 7.8 Hz, 1H), 7.34-7.32 |
| | | (m, 1H), 4.96 (s, 2H), 4.83 (brd, J = 5.4 Hz, 2H), 4.28-4.26 (m, 1H), 4.01-3.99 (m, |
| | | 1H), 3.88 (br d, J = 12.4 Hz, 2H), 3.78-3.69 (m, 2H), 3.67-3.60 (m, 1H), 2.60- |
| | | 2.58 (m, 2H), 1.18-1.11 (m, 9H) ppm |
| 611 | 544.3 | 1H NMR (400 MHz, DMSO-d6) δ = 9.67-9.59 (m, 1H), 9.36 (s, 1H), 8.63 (s, 2H), |
| | | 8.54 (d, J = 2.0 Hz, 1H), 8.38 (s, 1H), 8.30-8.23 (m, 1H), 7.81-7.70 (m, 3H), 7.44 |
| | | (d, J = 8.4 Hz, 1H), 4.98 (s, 2H), 4.81 (d, J = 6.0 Hz, 2H), 4.25-4.21 (m, 2H), 3.71- |
| | | 3.66 (m, 2H), 3.42 (br d, J = 5.6 Hz, 2H), 3.21 (s, 3H), 3.00-2.86 (m, 1H), 2.03- |
| | | 1.91 (m, 1H), 1.73-1.61 (m, 1H), 1.25 (d, J = 6.9 Hz, 3H) ppm |
| 612 | 544.2 | 1H NMR (400 MHz, DMSO-d6) δ = 9.63-9.61 (m, 1H), 9.35 (s, 1H), 8.62 (s, 2H), |
| | | 8.53 (d, J = 1.8 Hz, 1H), 8.39 (br s, 1H), 8.26-8.24 (m, 1H), 7.80-7.68 (m, 3H), |
| | | 7.33 (d, J = 7.4 Hz, 1H), 4.97 (s, 2H), 4.80 (d, J = 5.8 Hz, 2H), 4.27-4.17 (m, 2H), |
| | | 3.72-3.63 (m, 2H), 3.41-3.38 (m, 1H), 3.19 (s, 3H), 3.08-3.06 (m, 1H), 2.89- |
| | | 2.74 (m, 1H), 2.46-2.41 (m, 1H), 2.14-2.00 (m, 1H), 1.02 (d, J = 6.6 Hz, 3H) ppm |
| 613 | 575.3 | 1H NMR (400 MHz, DMSO-d6) δ = 9.63-9.60 (m, 1H), 9.37 (s, 1H), 8.57-8.47 (m, |
| | | 2H), 8.25-8.23 (m, 1H), 7.85 (d, J = 8.4 Hz, 1H), 7.78-7.69 (m, 2H), 7.09-6.96 |
| | | (m, 2H), 6.92-6.83 (m, 1H), 4.97 (s, 2H), 4.81 (d, J = 5.6 Hz, 2H), 4.31-4.17 (m, |
| | | 3H), 4.12-4.08 (m, 1H), 3.82-3.74 (m, 1H), 3.71-3.64 (m, 2H), 3.44-3.38 (m, |
| | | 2H), 3.30 (s, 3H), 2.94 (s, 3H) ppm |
| 614 | 575.3 | 1H NMR (400 MHz, DMSO-d6) δ = 9.63-9.60 (m, 1H), 9.37 (s, 1H), 8.58-8.47 (m, |
| | | 2H), 8.25-8.23 (m, 1H), 7.85 (d, J = 8.4 Hz, 1H), 7.79-7.69 (m, 2H), 7.09-6.96 |
| | | (m, 2H), 6.88-6.85 (m, 1H), 4.97 (s, 2H), 4.81 (d, J = 5.6 Hz, 2H), 4.30-4.19 (m, |
| | | 3H), 4.12-4.08 (m, 1H), 3.84-3.73 (m, 1H), 3.71-3.63 (m, 2H), 3.44-3.38 (m, |
| | | 2H), 3.30 (s, 3H), 2.94 (s, 3H) ppm |
| 615 | 545.2 | 1H NMR (400 MHz, DMSO-d6) δ = 9.57-9.54 (m, 1H), 9.03 (s, 1H), 9.12-8.93 (m, |
| | | 1H), 8.53 (d, J = 2.0 Hz, 1H), 8.26-8.24 (m, 1H), 8.17 (d, J = 9.2 Hz, 1H), 7.94 (d, |
| | | J = 9.2 Hz, 1H), 7.73 (d, J = 8.0 Hz, 1H), 7.55-7.54 (m, 1H), 7.46 (s, 1H), 7.11- |
| | | 7.09 (m, 1H), 7.00-6.97 (m, 1H), 4.98 (s, 2H), 4.72 (d, J = 5.6 Hz, 2H), 4.26-4.21 |
| | | (m, 4H), 3.76-3.62 (m, 2H), 3.43-3.37 (m, 4H), 1.13-1.05 (m, 3H) ppm |
| 616 | 558.3 | 1H NMR (400 MHz, DMSO-d6) δ = 9.63-9.55 (m, 1H), 9.10 (s, 1H), 8.53 (d, J = |
| | | 1.8 Hz, 1H), 8.46 (s, 2H), 8.30 (d, J = 8.8 Hz, 1H), 8.25-8.23 (m, 1H), 8.22-8.18 |
| | | (m, 1H), 7.73 (d, J = 8.0 Hz, 1H), 7.49 (s, 1H), 7.21 (d, J = 8.0 Hz, 1H), 6.98 (d, J = |
| | | 8.0 Hz, 1H), 4.98 (s, 2H), 4.73 (d, J = 5.6 Hz, 2H), 4.31-4.27 (m, 4H), 4.25-4.19 |
| | | (m, 2H), 3.70-3.65 (m, 2H), 2.04-1.97 (m, 1H), 0.89-0.84 (m, 2H), 0.81-0.76 (m, 2H) ppm |
| 617 | 582.3 | 1H NMR (400 MHz, DMSO-d6) δ = 9.61-9.59 (m, 1H), 9.14 (s, 1H), 8.53 (d, J = |
| | | 1.8 Hz, 1H), 8.37 (d, J = 9.4 Hz, 2H), 8.30-8.23 (m, 2H), 7.73 (d, J = 7.8 Hz, 1H), |
| | | 7.53 (s, 1H), 7.45 (d, J = 8.2 Hz, 1H), 7.33 (d, J = 8.2 Hz, 1H), 4.98 (s, 2H), 4.75 (d, |
| | | J = 5.6 Hz, 2H), 4.44-4.38 (m, 2H), 4.36 (br d, J = 4.8 Hz, 2H), 4.25-4.19 (m, 2H), |
| | | 3.71-3.65 (m, 2H), 1.96-1.94 (m, 3H) ppm |
| 618 | 545.2 | 1H NMR (400 MHz, DMSO-d6) δ = 9.59-9.56 (m, 1H), 9.02 (s, 1H), 8.54 (d, J = |
| | | 1.6 Hz, 1H), 8.40 (s, 1H), 8.26 (d, J = 7.6 Hz, 1H), 8.16 (d, J = 9.2 Hz, 1H), 7.95 (d, |
| | | J = 9.2 Hz, 1H), 7.74 (d, J = 7.6 Hz, 1H), 7.53-7.38 (m, 2H), 6.92 (d, J = 1.6 Hz, |
| | | 1H), 4.99 (s, 2H), 4.72 (d, J = 5.6 Hz, 2H), 4.27-4.25 (m, 4H), 3.73-3.64 (m, 2H), |
| | | 3.39-3.38 (m, 2H), 2.93 (s, 3H), 2.24 (s, 3H) ppm |
| 619 | 548.2 | 1H NMR (400 MHz, DMSO-d6) δ = 9.58-9.55 (m, 1H), 9.06 (s, 1H), 8.53 (s, 1H), |
| | | 8.40 (d, J = 4.0 Hz, 1H), 8.31-8.16 (m, 3H), 7.79-7.68 (m, 2H), 7.46 (s, 1H), 7.08 |
| | | (d, J = 2.4 Hz, 1H), 4.98 (s, 2H), 4.72 (d, J = 5.6 Hz, 2H), 4.33 (s, 4H), 4.26-4.20 |
| | | (m, 2H), 3.81 (s, 3H), 3.70-3.65 (m, 2H) ppm |
| 620 | 582.2 | 1H NMR (400 MHz, METHANOL-d4) δ = 9.03 (s, 1H), 8.61 (d, J = 1.8 Hz, 1H), 8.52- |
| | | 8.37 (m, 1H), 8.23-8.14 (m, 2H), 8.07 (d, J = 5.7 Hz, 1H), 7.87 (d, J = 9.2 Hz, |
| | | 1H), 7.69-7.60 (m, 2H), 7.31-6.90 (m, 1H), 6.88 (d, J = 5.6 Hz, 1H), 5.04 (s, 2H), |
| | | 4.85-4.85 (m, 2H), 4.39-4.31 (m, 2H), 4.23-4.21 (m, 2H), 3.56-3.47 (m, 2H), |
| | | 2.86 (t, J = 6.7 Hz, 2H), 2.09-2.00 (m, 2H) ppm |
| 621 | 586.3 | 1H NMR (400 MHz, Methanol-d4) δ 9.30 (s, 1H), 8.69-8.57 (m, 3H), 8.25-8.19 |
| | | (m, 1H), 7.96 (s, 2H), 7.88 (d, J = 7.3 Hz, 1H), 7.74-7.60 (m, 2H), 6.98 (d, J = 8.5 |
| | | Hz, 1H), 5.05 (s, 3H), 4.93 (d, J = 3.1 Hz, 3H), 4.61 (t, J = 7.8 Hz, 2H), 4.38-4.31 |
| | | (m, 3H), 3.83-3.67 (m, 3H), 3.53 (dd, J = 5.7, 4.0 Hz, 3H), 2.52 (t, J = 7.8 Hz, 2H), |
| | | 2.06-1.95 (m, 6H), 1.15 (d, J = 6.1 Hz, 3H). |
| 622 | 588.3 | 1H NMR (400 MHz, Methanol-d4) δ 9.30 (s, 1H), 8.69-8.57 (m, 3H), 8.25-8.19 |
| | | (m, 1H), 7.96 (s, 2H), 7.88 (d, J = 7.3 Hz, 1H), 7.74-7.60 (m, 2H), 6.98 (d, J = 8.5 |
| | | Hz, 1H), 5.05 (s, 3H), 4.93 (d, J = 3.1 Hz, 3H), 4.61 (t, J = 7.8 Hz, 2H), 4.38-4.31 |
| | | (m, 3H), 3.83-3.67 (m, 3H), 3.53 (dd, J = 5.7, 4.0 Hz, 3H), 2.52 (t, J = 7.8 Hz, 2H), |
| | | 2.06-1.95 (m, 6H), 1.15 (d, J = 6.1 Hz, 3H). |
| 623 | 587.3 | 1H NMR (400 MHz, DMSO-d6) δ 9.58 (t, J = 5.8 Hz, 1H), 9.35 (s, 1H), 8.64 (d, J = |
| | | 1.7 Hz, 1H), 8.56 (d, J = 1.8 Hz, 1H), 8.38 (dd, J = 8.6, 1.7 Hz, 1H), 8.31 (dd, J = |
| | | 7.8, 1.9 Hz, 1H), 8.22 (d, J = 8.7 Hz, 1H), 7.89 (s, 1H), 7.76 (d, J = 7.8 Hz, 1H), |
| | | 7.73 (dd, J = 8.5, 7.5 Hz, 1H), 7.49 (d, J = 7.5 Hz, 1H), 6.94 (d, J = 8.5 Hz, 1H), |
| | | 5.00 (s, 2H), 4.84-4.78 (m, 2H), 4.39-4.29 (m, 3H), 4.06 (dd, J = 13.3, 5.8 Hz, |
| | | 1H), 3.68 (dddd, J = 14.0, 7.8, 5.9, 2.3 Hz, 3H), 2.54-2.46 (m, 1H), 1.24 (d, J = |
| | | 6.2 Hz, 6H), 1.20 (d, J = 7.0 Hz, 3H). |
| 624 | 605.3 | 1H NMR (600 MHz, DMSO-d6) δ 9.65 (t, J = 5.8 Hz, 1H), 9.32 (s, 1H), 8.62 (d, J = |
| | | 1.7 Hz, 1H), 8.40 (d, J = 1.7 Hz, 1H), 8.35 (dd, J = 8.5, 1.7 Hz, 1H), 8.20 (dd, J = |
| | | 9.1, 3.3 Hz, 2H), 7.89 (s, 1H), 7.71 (t, J = 7.9 Hz, 1H), 7.46 (d, J = 7.4 Hz, 1H), 6.91 |
| | | (d, J = 8.5 Hz, 1H), 5.08 (d, J = 14.4 Hz, 1H), 4.96 (d, J = 14.4 Hz, 1H), 4.83-4.73 |
| | | (m, 2H), 4.31 (ddd, J = 13.1, 10.1, 2.4 Hz, 3H), 4.04 (dd, J = 13.3, 5.8 Hz, 1H), 3.76- |
| | | 3.67 (m, 1H), 3.69-3.63 (m, 1H), 2.50-2.45 (m, 1H), 1.28-1.23 (m, 1H), 1.21 |
| | | (d, J = 6.2 Hz, 6H), 1.18 (d, J = 7.0 Hz, 3H), |
| 625 | 551.2 | 1H NMR (400 MHz, DMSO-d6) δ = 9.58-9.56 (m, 1H), 9.02 (s, 1H), 8.51 (d, J = |
| | | 1.6 Hz, 1H), 8.40 (s, 1H), 8.29-8.17 (m, 2H), 7.72 (d, J = 7.6 Hz, 1H), 7.50 (d, J = |
| | | 7.6 Hz, 1H), 7.45-7.38 (m, 2H), 7.33 (d, J = 9.2 Hz, 1H), 4.97 (s, 2H), 4.70 (d, J = |
| | | 5.6 Hz, 2H), 4.27-4.18 (m, 2H), 3.98-3.94 (m, 2H), 3.70-3.64 (m, 2H), 2.72- |
| | | 2.69(m, 2H), 1.94-1.88 (m, 2H) ppm |
| 626 | 574.3 | 1H NMR (400 MHz, DMSO-d6) δ = 9.56-9.55 (m, 1H), 9.07-8.97 (m, 1H), 8.52 |
| | | (d, J = 1.8 Hz, 1H), 8.28-8.21 (m, 1H), 8.14 (d, J = 9.4 Hz, 1H), 7.99 (d, J = 9.2 |
| | | Hz, 1H), 7.72 (d, J = 7.8 Hz, 1H), 7.44 (d, J = 9.6 Hz, 2H), 4.97 (s, 2H), 4.72 (d, J = |
| | | 5.6 Hz, 2H), 4.38 (s, 2H), 4.25-4.18 (m, 2H), 4.15-4.08 (m, 2H), 3.71-3.64 (m, |
| | | 2H), 3.27 (s, 3H), 2.78 (t, J = 6.3 Hz, 2H), 2.27 (s, 3H), 1.93-1.91 (m, 2H) ppm |
| 627 | 588.3 | 1H NMR (400 MHz, DMSO-d6) δ = 9.56-9.55 (m, 1H), 9.41 (s, 1H), 8.72-8.60 (m, |
| | | 2H), 8.55 (d, J = 1.6 Hz, 1H), 8.45 (br s, 1H), 8.28-8.27 (m, 1H), 7.93 (d, J = 7.2 |
| | | Hz, 1H), 7.82 (s, 1H), 7.78-7.66 (m, 2H), 7.04 (d, J = 8.4 Hz, 1H), 4.82 (br d, J = |
| | | 5.6 Hz, 2H), 4.32 (brd, J = 12.2 Hz, 2H), 4.05-3.95 (m, 2H), 3.78-3.76 (m, 2H), |
| | | 3.72-3.63 (m, 2H), 3.57-3.46 (m, 4H), 2.63-2.54 (m, 2H), 1.22-1.20 (d, J = 6.2 |
| | | Hz, 6H) ppm |
| 628 | 588.3 | 1H NMR (400 MHz, DMSO-d6) δ = 9.63-9.62 (m, 1H), 9.40 (s, 1H), 8.73-8.56 (m, |
| | | 3H), 8.30 (d, J = 1.6 Hz, 1H), 7.91 (d, J = 7.6 Hz, 1H), 7.82 (s, 1H), 7.78-7.72 (m, |
| | | 2H), 7.03 (d, J = 8.4 Hz, 1H), 4.88 (s, 2H), 4.82 (d, J = 5.6 Hz, 2H), 4.31 (d, J = |
| | | 11.6 Hz, 2H), 3.68 (d, J = 10.4 Hz, 2H), 3.62-3.60 (m, 2H), 3.55-3.48 (m, 2H), |
| | | 2.53 (s, 2H), 2.29-2.26 (m, 2H), 1.21 (d, J = 6.0 Hz, 6H) ppm |
| 629 | 560.1 | 1H NMR (400 MHz, DMSO-d6) δ = 9.58-5.55 (m, 1H), 9.04 (s, 1H), 8.53 (d, J = |
| | | 1.6 Hz, 1H), 8.26-8.24 (m, 1H), 8.14 (d, J = 9.2 Hz, 1H), 7.98 (s, 1H), 7.89 (d, J = |
| | | 9.2 Hz, 1H), 7.73 (d, J = 7.6 Hz, 1H), 7.46 (s, 1H), 4.98 (s, 2H), 4.72 (d, J = 5.6 Hz, |
| | | 2H), 4.27-4.17 (m, 2H), 4.14-4.08 (m, 2H), 3.79 (s, 3H), 3.71-3.64 (m, 2H), 2.79- |
| | | 2.76 (m, 2H), 2.19 (s, 3H), 2.00-1.86 (m, 2H) ppm |
| 630 | 586.3 | 1H NMR (400 MHz, DMSO-d6) δ = 9.57-9.56 (m, 1H), 9.07 (s, 1H), 8.53 (d, J = |
| | | 1.8 Hz, 1H), 8.42 (s, 1H), 8.25-8.24 (m, 1H), 8.18 (d, J = 9.2 Hz, 1H), 7.95 (d, J = |
| | | 9.2 Hz, 1H), 7.73 (d, J = 7.8 Hz, 1H), 7.56-7.46 (m, 2H), 6.96 (d, J = |
| | | 7.6 Hz, 1H), 4.98 (s, 2H), 4.73 (d, J = 5.6 Hz, 2H), 4.27-4.18 (m, 2H), 4.16-4.10 |
| | | (m, 2H), 4.02 (t, J = 7.8 Hz, 1H), 3.85-3.72 (m, 2H), 3.71-3.61 (m, 3H), 3.44- |
| | | 3.42 (m, 1H), 2.79-2.77 (m, 2H), 2.23-2.15 (m, 1H), 2.10-2.03 (m, 1H), 1.95- |
| | | 1.93 (m, 2H) ppm |
| 631 | 628.4 | 1H NMR (400 MHz, DMSO-d6) δ = 9.68-9.67 (m, 1H), 9.41 (s, 1H), 8.71-8.60 (m, |
| | | 2H), 8.43 (d, J = 1.6 Hz, 1H), 8.16 (s, 1H), 7.91 (d, J = 7.2 Hz, 1H), 7.81 (s, 1H), |
| | | 7.74-7.72 (m, 1H), 7.03 (d, J = 8.2 Hz, 1H), 5.00 (s, 2H), 4.83 (d, J = 5.8 Hz, 2H), |
| | | 4.37-4.26 (m, 2H), 4.24-4.14 (m, 2H), 3.95-3.83 (m, 1H), 3.72-3.63 (m, 4H), |
| | | 2.56-2.54 (m, 2H), 2.37 (br s, 1H), 2.28-2.19 (m, 2H), 2.07-1.99 (m, 1H), 1.86- |
| | | 1.76 (m, 1H), 1.21 (d, J = 6.2 Hz, 6H) ppm |
| 632 | 570.1 | 1H NMR (400 MHz, DMSO-d6) δ = 9.57-9.54 (m, 1H), 9.04 (s, 1H), 8.53 (d, J = |
| | | 1.6 Hz, 1H), 8.25-8.24 (m, 1H), 8.15 (d, J = 9.2 Hz, 1H), 7.79 (d, J = 9.2 Hz, 1H), |
| | | 7.73 (d, J = 7.6 Hz, 1H), 7.45 (s, 1H), 7.32 (s, 1H), 4.97 (s, 2H), 4.72 (d, J = 6.0 Hz, |
| | | 2H), 4.28-4.19 (m, 2H), 4.12-4.05 (m, 2H), 3.74-3.62 (m, 2H), 2.74-2.71 (m, |
| | | 2H), 2.33 (s, 3H), 2.12-2.04 (m, 1H), 1.92-1.89 (m, 2H), 0.88-0.76 (m, 4H) ppm |
| 633 | 546.2 | 1H NMR (400 MHz, DMSO-d6) δ = 9.58-9.55 (m, 1H), 9.07 (s, 1H), 8.53 (d, J = |
| | | 1.6 Hz, 1H), 8.26-8.24 (m, 1H), 8.18 (d, J = 9.2 Hz, 1H), 8.19-8.14 (m, 1H), 7.91 |
| | | (d, J = 9.2 Hz, 1H), 7.73 (d, J = 8.0 Hz, 1H), 7.63 (d, J = 6.8 Hz, 1H), 7.51 (s, 1H), |
| | | 7.08-7.06 (m, 1H), 4.98 (s, 2H), 4.74 (d, J = 5.6 Hz, 2H), 4.60-4.52 (m, 1H), 4.27- |
| | | 4.19 (m, 2H), 3.99-3.90 (m, 2H), 3.73-3.62 (m, 2H), 3.17 (s, 3H), 3.08-3.06 (m, |
| | | 1H), 2.96-2.88 (m, 1H) ppm |
| 634 | 567.2 | 1H NMR (400 MHz, DMSO-d6) δ = 9.59-9.57 (m, 1H), 9.07 (s, 1H), 8.51 (d, J = |
| | | 1.6 Hz, 1H), 8.34 (d, J = 8.8 Hz, 1H), 8.25-8.23 (m, 1H), 7.76-7.70 (m, 2H), 7.53 |
| | | (d, J = 9.2 Hz, 1H), 7.43 (s, 1H), 7.27-7.22 (m, 1H), 7.08-6.79 (m, 2H), 4.97 (s, |
| | | 2H), 4.72 (d, J = 6.0 Hz, 2H), 4.37-4.31 (m, 2H), 4.26-4.19 (m, 4H), 3.71-3.63 |
| | | (m, 2H) ppm |
| 635 | 588.4 | 1H NMR (400 MHz, DMSO-d6) δ = 9.58-9.56 (m, 1H), 9.06 (s, 1H), 8.53 (d, J = |
| | | 1.8 Hz, 1H), 8.25-8.23 (m, 1H), 8.18 (d, J = 9.2 Hz, 1H), 8.14 (s, 1H), 7.96 (d, J = |
| | | 9.2 Hz, 1H), 7.73 (d, J = 7.8 Hz, 1H), 7.59 (d, J = 7.6 Hz, 1H), 7.48 (s, |
| | | 1H), 7.05 (d, J = 7.6 Hz, 1H), 4.98 (s, 2H), 4.73 (d, J = 6.0 Hz, 2H), 4.32-4.31 (m, |
| | | 1H), 4.23-4.21 (m, 2H), 4.21-4.05 (m, 2H), 3.71-3.63 (m, 2H), 3.47-3.35 (m, |
| | | 2H), 2.81-2.80 (m, 2H), 2.03-1.86 (m, 2H), 1.34 (d, J = 6.6 Hz, |
| | | 3H), 1.12-1.10 (m, 3H) ppm |
| 636 | 588.4 | 1H NMR (400 MHz, DMSO-d6) δ = 9.58-9.56 (m, 1H), 9.06 (s, 1H), 8.53 (d, J = 1.8 |
| | | Hz, 1H), 8.38 (s, 1H), 8.25-8.23 (m, 1H), 8.18 (d, J = 9.2 Hz, 1H), 7.96 (d, J = 9.4 |
| | | Hz, 1H), 7.73 (d, J = 7.8 Hz, 1H), 7.59 (d, J = 7.6 Hz, 1H), 7.48 (s, |
| | | 1H), 7.05 (d, J = 7.6 Hz, 1H), 4.98 (s, 2H), 4.73 (d, J = 5.8 Hz, 2H), 4.32-4.30 (m, |
| | | 1H), 4.27-4.20 (m, 2H), 4.20-4.03 (m, 2H), 3.76-3.63 (m, 2H), 3.46-3.39 (m, |
| | | 2H), 2.81-2.80 (m, 2H), 2.03-1.87 (m, 2H), 1.34 (d, J = 6.4 Hz, 3H), 1.12-1.10 |
| | | (m, 3H) ppm |
| 637 | 560.2 | 1H NMR (400 MHz, DMSO-d6) δ = 9.57-9.54 (m, 1H), 8.99 (s, 1H), 8.53 (d, J = |
| | | 2.0 Hz, 1H), 8.26-8.24 (m, 1H), 8.10 (d, J = 9.2 Hz, 1H), 7.90 (s, 1H), 7.78 (d, J = |
| | | 9.6 Hz, 1H), 7.73 (d, J = 7.6 Hz, 1H), 7.43 (s, 1H), 4.98 (s, 2H), 4.71 (d, J = 5.6 Hz, |
| | | 2H), 4.29-4.18 (m, 2H), 4.15-4.06 (m, 2H), 3.87 (s, 3H), 3.71-3.65 (m, 2H), 2.75- |
| | | 2.71 (m, 2H), 2.13 (s, 3H), 1.98-1.89 (m, 2H) ppm |
| 638 | 575.3 | 1H NMR (400 MHz, CHLOROFORM-d) δ = 9.26 (s, 1H), 8.57 (d, J = 2.0 Hz, 1H), |
| | | 8.29 (d, J = 8.8 Hz, 1H), 8.22-8.19 (m, 1H), 8.15-8.07 (m, 2H), 7.92 (s, 1H), 7.49 |
| | | (d, J = 7.6 Hz, 1H), 7.20-7.17 (m, 1H), 7.04-7.01 (m, 1H), 6.76-6.73 (m, 1H), |
| | | 5.05-5.01 (m, 4H), 4.43-4.37 (m, 3H), 4.19-3.99 (m, 1H), 3.55-3.48 (m, 3H), |
| | | 3.43-3.40 (m, 2H), 3.38 (s, 3H), 3.06 (s, 3H) ppm |
| 639 | 575.3 | 1H NMR (400 MHz, CHLOROFORM-d) δ = 9.25 (s, 1H), 8.55 (d, J = 2.0 Hz, 1H), |
| | | 8.27 (d, J = 8.8 Hz, 1H), 8.21-8.19 (m, 1H), 8.09-8.04 (m, 2H), 7.80 (s, 1H), 7.49 |
| | | (d, J = 7.6 Hz, 1H), 7.20-7.17 (m, 1H), 7.04-7.01 (m, 1H), 6.76-6.73 (m, 1H), |
| | | 5.05-4.98 (m, 4H), 4.42-4.37 (m, 3H), 4.12-3.99 (m, 1H), 3.56-3.48 (m, 3H), |
| | | 3.43-3.40 (m, 2H), 3.38 (s, 3H), 3.06 (s, 3H) ppm |
| 640 | 521.3 | 1H NMR (400 MHz, DMSO-d6) δ = 9.49-9.48 (m, 1H), 8.84 (s, 1H), 8.51 (d, J = |
| | | 1.8 Hz, 1H), 8.32-8.17 (m, 1H), 8.08 (d, J = 9.2 Hz, 1H), 7.72 (d, J = 7.8 Hz, 1H), |
| | | 7.37-7.14 (m, 2H), 5.05-4.91 (m, 2H), 4.69-4.61 (m, 2H), 4.26-4.16 (m, 2H), |
| | | 3.73-3.62 (m, 2H), 3.02-2.77 (m, 1H), 2.46-2.42 (m, 2H), 1.87-1.54 (m, 7H), |
| | | 1.49-1.26 (m, 6H) ppm |
| 641 | 605.2 | 1H NMR (400 MHz, DMSO-d6) δ = 9.42-9.36 (m, 2H), 8.67-8.63 (m, 2H), 8.23 (d, |
| | | J = 1.6 Hz, 1H), 7.97-7.90 (m, 2H), 7.79-7.70 (m, 2H), 7.03 (d, J = 8.4 Hz, 1H), |
| | | 4.77 (br d, J = 5.6 Hz, 2H), 4.32 (br d, J = 12 Hz, 2H), 3.87-3.84 (m, 1H), 3.79- |
| | | 3.63 (m, 4H), 3.25 (d, J = 5.6 Hz, 3H), 2.54-2.52 (m, 2H), 1.30 (d, J = 6.8 Hz, 3H), |
| | | 1.22 (d, J = 6.0 Hz, 6H) ppm |
| 642 | 605.3 | 1H NMR (400 MHz, DMSO-d6) δ = 9.42-9.36 (m, 2H), 8.67-8.63 (m, 2H), 8.22 (d, |
| | | J = 1.6 Hz, 1H), 7.97-7.90 (m, 2H), 7.79-7.70 (m, 2H), 7.02 (d, J = 8.4 Hz, 1H), |
| | | 4.77 (br d, J = 5.6 Hz, 2H), 4.31 (br d, J = 12 Hz, 2H), 3.86-3.84 (m, 1H), 3.79- |
| | | 3.63 (m, 4H), 3.25 (d, J = 5.6 Hz, 3H), 2.55-2.52 (m, 2H), 1.30 (d, J = 6.8 Hz, 3H), |
| | | 1.22 (d, J = 6.0 Hz, 6H) ppm |
| 643 | 582.3 | 1H NMR (400 MHz, METHANOL-d4) δ = 9.05 (s, 1H), 8.61 (d, J = 1.6 Hz, 1H), 8.51- |
| | | 8.39 (m, 1H), 8.26-8.16 (m, 2H), 7.96 (d, J = 9.2 Hz, 1H), 7.71-7.59 (m, 3H), |
| | | 7.45-7.04 (m, 1H), 6.56 (d, J = 7.8 Hz, 1H), 5.04 (s, 2H), 4.85-4.84 (m, 2H), 4.40- |
| | | 4.30 (m, 2H), 4.24-4.17 (m, 2H), 3.57-3.49 (m, 2H), 2.87 (br t, J = 6.6 Hz, 2H), |
| | | 2.12-2.01 (m, 2H) ppm |
| 644 | 571.2 | 1H NMR (400 MHz, DMSO-d6) δ = 9.58-9.55 (m, 1H), 8.99 (s, 1H), 8.52 (d, J = |
| | | 2.0 Hz, 1H), 8.40 (s, 1H), 8.25-8.23 (m, 1H), 8.16 (d, J = 9.2 Hz, 1H), 7.73 (d, J = |
| | | 7.6 Hz, 1H), 7.48 (d, J = 9.2 Hz, 1H), 7.38 (s, 1H), 7.14 (d, J = 8.4 Hz, 1H), 7.08 (d, |
| | | J = 2.4 Hz, 1H), 6.75-6.73 (m, 1H), 4.98 (s, 2H), 4.70 (d, J = 5.6 Hz, 2H), 4.28- |
| | | 4.19 (m, 2H), 3.99-3.95 (m, 2H), 3.76-3.71 (m, 1H), 3.70-3.65 (m, 2H), 2.67- |
| | | 2.64 (m, 2H), 1.93-1.86 (m, 2H), 0.730.57 (m, 4H) ppm |
| 645 | 551.8 | 1H NMR (400 MHz, DMSO-d6) δ = 9.59 (s, 1H), 9.13 (s, 1H), 8.53 (d, J = 1.8 Hz, |
| | | 1H), 8.35-8.31 (m, 1H), 8.27-8.20 (m, 2H), 7.95 (d, J = 2.4 Hz, 1H), 7.74 (d, J = |
| | | 7.8 Hz, 1H), 7.55 (d, J = 2.4 Hz, 1H), 7.52 (s, 1H), 4.98 (s, 2H), 4.74 (d, J = 5.6 Hz, |
| | | 2H), 4.36-4.34 (m, 4H), 4.23-4.21 (m, 2H), 3.72-3.65 (m, 2H) ppm |
| 646 | 565.8 | 1H NMR (400 MHz, DMSO-d6) δ = 9.59 (s, 1H), 9.13 (s, 1H), 8.52 (d, J = 1.6 Hz, |
| | | 1H), 8.39 (s, 1H), 8.35-8.31 (m, 1H), 8.27-8.26 (z, 1H), 8.23 (d, J = 9.2 Hz, 1H), |
| | | 8.24-8.21 (m, 1H), 7.95 (d, J = 2.4 Hz, 1H), 7.74 (d, J = 7.8 Hz, 1H), 7.57-7.52 |
| | | (m, 2H), 4.96 (s, 2H), 4.74 (br d, J = 5.6 Hz, 2H), 4.41-4.32 (m, 4H), 4.29-4.28 |
| | | (m, 1H), 4.03 (s, 1H), 3.69-3.61 (m, 1H), 1.16 (d, J = 7.0 Hz, 3H) ppm |
| 647 | 531.4 | 1H NMR (400 MHz, DMSO-d6) δ = 9.54-9.53 (m, 1H), 9.02 (s, 1H), 8.52 (d, J = |
| | | 2.0 Hz, 1H), 8.41-8.28 (m, 2H), 8.24-8.23 (m, 1H), 8.19 (s, 1H), 7.73 (d, J = 7.8 |
| | | Hz, 1H), 7.51 (s, 1H), 7.31-7.14 (m, 2H), 6.67 (d, J = 8.2 Hz, 1H), 4.98 |
| | | (s, 2H), 4.72 (d, J = 5.8 Hz, 2H), 4.29-4.19 (m, 4H), 3.81 (s, 3H), 3.70-3.65 (m, |
| | | 2H), 3.11-3.09 (m, 2H) ppm |
| 648 | 592.3 | 1H NMR (400 MHz, DMSO-d6) δ = 9.41 (s, 1H), 9.35-9.33 (m, 1H), 8.69-8.62 (m, |
| | | 2H), 8.25 (d, J = 6.8 Hz, 1H), 7.92 (d, J = 7.6 Hz, 1H), 7.87 (s, 1H), 7.80-7.69 (m, |
| | | 2H), 7.04 (d, J = 8.4 Hz, 1H), 4.95 (s, 2H), 4.82 (br d, J = 6.0 Hz, 2H), 4.32 (br d, |
| | | J = 11.6 Hz, 2H), 4.26-4.18 (m, 2H), 3.73-3.62 (m, 4H), 2.54-2.53 (m, 2H), 1.22 |
| | | (d, J = 6.4 Hz, 6H) ppm |
| 649 | 545.2 | 1H NMR (400 MHz, DMSO-d6) δ = 9.57-9.55 (m, 1H), 9.03 (s, 1H), 8.53 (d, J = |
| | | 1.6 Hz, 1H), 8.27-8.25 (m, 1H), 8.18 (d, J = 9.2 Hz, 1H), 7.95 (d, J = 9.2 Hz, 1H), |
| | | 7.73 (d, J = 7.6 Hz, 1H), 7.56 (d, J = 3.6 Hz, 1H), 7.45 (s, 1H), 7.03-6.98 (m, 2H), |
| | | 4.98 (s, 2H), 4.78 (d, J = 13.2 Hz, 1H), 4.72 (d, J = 6.0 Hz, 2H), 4.30-4.18 (m, 2H), |
| | | 3.69-3.67 (m, 3H), 3.59 (d, J = 3.2 Hz, 1H), 2.91 (s, 3H), 1.00 (d, J = 6.4 Hz, 3H) ppm |
| 650 | 573.3 | 1H NMR (400 MHz, DMSO-d6) δ = 9.56-9.54 (m, 1H), 8.96 (s, 1H), 8.52 (d, J = |
| | | 1.6 Hz, 1H), 8.26 (d, J = 7.6 Hz, 1H), 8.19 (s, 1H), 8.06 (d, J = 9.2 Hz, 1H), 7.81 (d, |
| | | J = 9.2 Hz, 1H), 7.77-7.71 (m, 2H), 7.42 (s, 1H), 7.09 (d, J = 2.8 Hz, 1H), 4.96 (s, |
| | | 2H), 4.70 (d, J = 5.6 Hz, 2H), 4.29 (d, J = 13.2 Hz, 1H), 4.13-4.07 (m, 2H), 4.02- |
| | | 4.00 (m, 1H), 3.70-3.60 (m, 1H), 2.90 (s, 6H), 2.77-2.75 (m, 2H), 1.97-1.86 (m, |
| | | 2H), 1.16 (d, J = 7.2 Hz, 3H) ppm |
| 651 | 574.2 | 1H NMR (400 MHz, DMSO-d6) δ = 9.63-9.47 (m, 1H), 9.12 (s, 1H), 8.53 (d, J = |
| | | 1.8 Hz, 1H), 8.32 (s, 1H), 8.25-8.23 (m, 1H), 8.18 (d, J = 9.2 Hz, 1H), 7.98 (d, J = |
| | | 9.2 Hz, 1H), 7.73 (d, J = 7.8 Hz, 1H), 7.55-7.40 (m, 2H), 6.92 (d, J = 7.6 Hz, 1H), |
| | | 5.04-4.91 (m, 2H), 4.73 (br d, J = 5.6 Hz, 2H), 4.27-4.09 (m, 4H), 3.72-3.61 (m, |
| | | 4H), 3.25 (s, 3H), 2.84-2.83 (m, 2H), 2.78-2.76 (m, 2H), 2.02-1.86 (m, 2H) ppm |
| 652 | 507.2 | 1H NMR (400 MHz, DMSO-d6) δ = 9.51-9.50 (m, 1H), 8.93 (s, 1H), 8.85 (s, 1H), |
| | | 8.55-8.47 (m, 1H), 8.26-8.21 (m, 1H), 8.20 (s, 1H), 8.16-8.05 (m, 1H), 7.72 (d, |
| | | J = 7.8 Hz, 1H), 7.36-7.19 (m, 2H), 4.97 (s, 2H), 4.72-4.61 (m, 2H), 4.28-4.17 (m, |
| | | 2H), 3.89-3.79 (m, 1H), 3.72-3.64 (m, 2H), 3.53-3.43 (m, 1H), 3.02 (dt, J = 6.2, |
| | | 10.4 Hz, 1H), 2.87-2.71 (m, 1H), 2.03-1.88 (m, 1H), 1.84-1.53 (m, 6H), 1.47- |
| | | 1.13 (m, 3H) ppm |
| 653 | 531.2 | 1H NMR (400 MHz, DMSO-d6) δ = 9.60-9.50 (m, 1H), 8.98 (s, 1H), 8.67 (br d, |
| | | J = 8.8 Hz, 1H), 8.52 (s, 1H), 8.40 (br s, 2H), 8.29-8.22 (m, 2H), 7.73 (d, J = 7.8 Hz, |
| | | 1H), 7.47 (s, 1H), 7.20 (d, J = 9.2 Hz, 1H), 6.90 (d, J = 2.0 Hz, 1H), 6.80-6.79 (m, |
| | | 1H), 4.98 (s, 2H), 4.71 (brd, J = 6.0 Hz, 2H), 4.26-4.17 (m, 4H), 3.73 (s, 3H), 3.70- |
| | | 3.66 (m, 2H), 3.21 (br s, 2H) ppm |
| 654 | 535.1 | 1H NMR (400 MHz, DMSO-d6) δ = 9.57-9.50 (m, 1H), 9.15-9.01 (m, 1H), 8.82 |
| | | (d, J = 2.0 Hz, 1H), 8.54 (s, 1H), 8.38 (d, J = 9.2 Hz, 1H), 8.29-8.19 (m, 1H), 7.72 |
| | | (d, J = 7.8 Hz, 1H), 7.59-7.50 (m, 1H), 7.34-7.22 (m, 2H), 7.00-6.98 (m, 1H), |
| | | 5.05-4.90 (m, 2H), 4.74 (d, J = 5.8 Hz, 2H), 4.33-4.17 (m, 4H), 3.73-3.60 (m, |
| | | 2H), 3.27-3.20 (m, 2H) ppm |
| 655 | 602.4 | 1H NMR (400 MHz, DMSO-d6) δ = 9.61-9.60 (m, 1H), 9.40 (s, 1H), 8.72-8.57 (m, |
| | | 3H), 8.51-8.41 (m, 1H), 8.33-8.25 (m, 1H), 7.90 (d, J = 7.2 Hz, 1H), 7.80-7.70 |
| | | (m, 3H), 7.03 (d, J = 8.6 Hz, 1H), 4.82 (br d, J = 5.6 Hz, 2H), 4.31 (br d, J = 11.8 |
| | | Hz, 2H), 4.02-3.90 (m, 2H), 3.85-3.75 (m, 2H), 3.72-3.62 (m, 2H), 2.41-2.40 |
| | | (m, 2H), 1.64 (s, 6H), 1.21 (d, J = 6.2 Hz, 6H) ppm |
| 656 | 516.3 | 1H NMR (400 MHz, DMSO-d6) δ = 9.57-9.56 (m, 1H), 9.17-9.02 (m, 2H), 8.53 |
| | | (d, J = 1.8 Hz, 1H), 8.36 (d, J = 9.2 Hz, 1H), 8.25-8.24 (m, 1H), 8.21 (br s, 1H), |
| | | 7.97 (s, 1H), 7.73 (d, J = 7.8 Hz, 1H), 7.56-7.42 (m, 2H), 4.98 (s, 2H), 4.72 (d, J = |
| | | 5.6 Hz, 2H), 4.32-4.31 (m, 2H), 4.26-4.18 (m, 2H), 3.71-3.65 (m, 2H), 3.12- |
| | | 3.10 (m, 2H), 2.24 (s, 3H) ppm |
| 657 | 528 | 1H NMR (400 MHz, METHANOL-d4) δ = 9.36 (s, 1H), 8.86 (s, 1H), 8.74 (s, 1H), |
| | | 8.68-8.62 (m, 2H), 8.28-8.20 (m, 2H), 8.03 (s, 1H), 7.65 (d, J = 7.8 Hz, 1H), 7.09 |
| | | (s, 1H), 5.06 (s, 2H), 4.97 (s, 2H), 4.39-4.31 (m, 2H), 3.94 (s, 3H), 3.60-3.51 (m, |
| | | 2H), 2.61 (s, 3H) ppm |
| 658 | 546.3 | 1H NMR (400 MHz, DMSO-d6) δ = 9.59-9.58 (m, 1H), 9.09 (s, 1H), 8.53 (d, J = |
| | | 1.8 Hz, 1H), 8.42-8.39 (m, 1H), 8.38 (br s, 1H), 8.32-8.23 (m, 2H), 7.73 (d, J = |
| | | 7.8 Hz, 1H), 7.49 (s, 1H), 7.25 (d, J = 8.2 Hz, 1H), 6.92 (d, J = 7.9 Hz, 1H), 4.98 (s, |
| | | 2H), 4.74 (d, J = 5.8 Hz, 2H), 4.32 (s, 4H), 4.26-4.19 (m, 2H), 3.73-3.65 (m, 2H), |
| | | 2.67-2.62 (m, 2H), 1.21 (t, J = 7.6 Hz, 3H) ppm |
| 659 | 586.4 | 1H NMR (400 MHz, DMSO-d6) δ = 9.58-9.57 (m, 1H), 9.06 (s, 1H), 8.53 (d, J = |
| | | 1.8 Hz, 1H), 8.44-8.33 (m, 1H), 8.25-8.23 (m, 1H), 8.18 (d, J = 9.2 Hz, 1H), 7.96 |
| | | (d, J = 9.2 Hz, 1H), 7.73 (d, J = 7.8 Hz, 1H), 7.56 (d, J = 7.6 Hz, 1H), |
| | | 7.48 (s, 1H), 7.04 (d, J = 7.8 Hz, 1H), 4.98 (s, 2H), 4.79-4.70 (m, 3H), 4.26-4.19 |
| | | (m, 2H), 4.18-4.06 (m, 2H), 3.98-3.91 (m, 1H), 3.85-3.77 (m, 1H), 3.72-3.66 |
| | | (m, 2H), 2.80-2.78 (m, 2H), 2.23-2.17 (m, 1H), 2.00-1.93 (m, 2H), 1.91-1.84 |
| | | (m, 3H) ppm |
| 660 | 594.3 | 1H NMR (400 MHz, DMSO-d6) δ = 9.58-9.57 (m, 1H), 9.12 (s, 1H), 8.53 (d, J = |
| | | 1.6 Hz, 1H), 8.30-8.23 (m, 2H), 7.95 (d, J = 9.2 Hz, 1H), 7.73 (d, J = 7.8 Hz, 1H), |
| | | 7.68 (s, 1H), 7.52 (s, 1H), 4.98 (s, 2H), 4.75 (br d, J = 5.6 Hz, 2H), 4.41 (s, 2H), |
| | | 4.25-4.20 (m, 2H), 4.17-4.11 (m, 2H), 3.71-3.66 (m, 2H), 3.35 (s, 3H), 2.84- |
| | | 2.82 (m, 2H), 2.01-1.91 (m, 2H) ppm |
| 661 | 544.3 | 1H NMR (400 MHz, DMSO-d6) δ = 9.62-9.61 (m, 1H), 9.36 (s, 1H), 8.62 (s, 2H), |
| | | 8.54 (d, J = 1.8 Hz, 1H), 8.37 (br s, 1H), 8.26-8.24 (m, 1H), 7.81-7.69 (m, 3H), |
| | | 7.34 (d, J = 7.6 Hz, 1H), 4.98 (s, 2H), 4.81 (d, J = 5.6 Hz, 2H), 4.27-4.20 (m, 2H), |
| | | 3.71-3.65 (m, 2H), 3.42-3.39 (m, 1H), 3.20 (s, 3H), 3.09-3.07 (m, 1H), 2.83- |
| | | 2.81 (m, 1H), 2.44 (br d, J = 9.8 Hz, 1H), 2.15-2.02 (m, 1H), 1.03 (d, J = 6.8 Hz, 3H) ppm |
| 662 | 544.3 | 1H NMR (400 MHz, DMSO-d6) δ = 9.62-9.61 (m, 1H), 9.36 (s, 1H), 8.62 (s, 2H), |
| | | 8.54 (d, J = 1.8 Hz, 1H), 8.37 (br s, 1H), 8.26-8.24 (m, 1H), 7.81-7.69 (m, 3H), |
| | | 7.34 (d, J = 7.6 Hz, 1H), 4.98 (s, 2H), 4.81 (d, J = 5.6 Hz, 2H), 4.27-4.20 (m, 2H), |
| | | 3.71-3.65 (m, 2H), 3.42-3.39 (m, 1H), 3.20 (s, 3H), 3.09-3.07 (m, 1H), 2.83- |
| | | 2.81 (m, 1H), 2.44 (br d, J = 9.8 Hz, 1H), 2.15-2.02 (m, 1H), 1.03 (d, J = 6.8 Hz, 3H) ppm |
| 663 | 600.3 | 1H NMR (400 MHz, DMSO-d6) δ = 9.59-9.58 (m, 1H), 9.06 (s, 1H), 8.53 (d, J = 1.6 |
| | | Hz, 1H), 8.45-8.39 (m, 1H), 8.25-8.24 (m, 1H), 8.18 (d, J = 9.2 Hz, 1H), 7.98 (d, |
| | | J = 9.2 Hz, 1H), 7.73 (d, J = 7.8 Hz, 1H), 7.60 (d, J = 7.8 Hz, 1H), |
| | | 7.48 (s, 1H), 7.04 (d, J = 7.6 Hz, 1H), 4.97 (s, 2H), 4.73 (br d, J = 5.6 Hz, 2H), 4.28- |
| | | 4.16 (m, 3H), 4.13-4.04 (m, 1H), 3.73-3.63 (m, 2H), 3.46 (br d, J = 8.2 Hz, 1H), |
| | | 3.19 (s, 3H), 2.82-2.80 (m, 2H), 1.96-1.94 (m, 2H), 1.16-1.00 |
| | | (m, 1H), 0.63-0.22 (m, 4H) ppm |
| 664 | 600.3 | 1H NMR (400 MHz, DMSO-d6) δ = 9.59-9.58 (m, 1H), 9.07 (s, 1H), 8.53 (d, J = |
| | | 1.6 Hz, 1H), 8.46 (s, 1H), 8.26-8.24 (m, 1H), 8.19 (d, J = 9.2 Hz, 1H), 7.98 (d, J = |
| | | 9.2 Hz, 1H), 7.74 (d, J = 8.2 Hz, 1H), 7.61 (d, J = 7.6 Hz, 1H), 7.49 (s, |
| | | 1H), 7.05 (d, J = 7.6 Hz, 1H), 4.98 (s, 2H), 4.74 (br d, J = 5.6 Hz, 2H), 4.27-4.18 |
| | | (m, 3H), 4.13-4.05 (m, 1H), 3.71-3.66 (m, 2H), 3.46 (s, 1H), 3.20 (s, 3H), 2.82- |
| | | 2.80 (m, 2H), 1.97-1.95 (m, 2H), 1.16-1.10 (m, 1H), 0.59- |
| | | 0.25 (m, 4H) ppm |
| 665 | 546.2 | 1H NMR (400 MHz, DMSO-d6) δ = 9.60-9.51 (m, 1H), 9.08 (s, 1H), 8.53 (d, J = |
| | | 2.0 Hz, 1H), 8.47-8.43 (m, 1H), 8.34-8.22 (m, 3H), 7.80 (d, J = 2.0 Hz, 1H), 7.73 |
| | | (d, J = 8.0 Hz, 1H), 7.48 (s, 1H), 7.23 (d, J = 2.0 Hz, 1H), 4.98 (s, 2H), 4.73 (d, J = |
| | | 5.6 Hz, 2H), 4.33 (s, 4H), 4.26-4.20 (m, 2H), 3.71-3.64 (m, 2H), 2.60-2.54 (m, |
| | | 2H), 1.20-1.16 (m, 3H) ppm |
| 666 | 571.3 | 1H NMR (400 MHz, DMSO-d6) δ = 9.56-9.53 (m, 1H), 8.96 (s, 1H), 8.53 (d, J = |
| | | 1.6 Hz, 1H), 8.25 (d, J = 7.6 Hz, 1H), 8.06 (d, J = 9.2 Hz, 1H), 7.75 (d, J = 8.4 Hz, |
| | | 2H), 7.44 (d, J = 2.8 Hz, 1H), 7.40 (s, 1H), 6.78 (d, J = 2.8 Hz, 1H), 4.98 (s, 2H), |
| | | 4.70 (d, J = 6.0 Hz, 2H), 4.27-4.20 (m, 2H), 4.12-4.05 (m, 2H), 3.86-3.84 (m, |
| | | 4H), 3.72-3.64 (m, 2H), 2.76-2.73 (m, 2H), 2.38-2.33 (m, 2H), 1.96-1.82 (m, 2H) ppm |
| 667 | 613.2 | 1H NMR (400 MHz, DMSO-d6) δ = 9.39 (s, 1H), 9.32-9.31 (m, 1H), 8.69-8.61 (m, |
| | | 2H), 8.35 (d, J = 2.0 Hz, 1H), 8.09-8.07 (m, 1H), 7.92 (d, J = 7.6 Hz, 1H), 7.77- |
| | | 7.71 (m, 2H), 7.39 (d, J = 9.2 Hz, 1H), 7.03 (d, J = 8.4 Hz, 1H), 4.78 (br d, J = 5.6 |
| | | Hz, 2H), 4.32 (br d, J = 11.2 Hz, 2H), 3.97-3.94 (m, 1H), 3.76-3.64 (m, 3H), 3.61- |
| | | 3.52 (m, 1H), 2.78-2.71 (m, 1H), 2.52 (m, 2H), 1.25 (d, J = 6.8 Hz, 3H), 1.22 (d, |
| | | J = 6.0 Hz, 6H), 0.99-0.97 (m, 2H), 0.74-0.73 (m, 2H) ppm |
| 668 | 574.2 | 1H NMR (400 MHz, DMSO-d6) δ = 9.63-9.62 (m, 1H), 9.39 (s, 1H), 8.66 (s, 2H), |
| | | 8.53 (d, J = 1.8 Hz, 1H), 8.28-8.27 (m, 1H), 7.89 (d, J = 7.2 Hz, 1H), 7.83 (s, 1H), |
| | | 7.76-7.69 (m, 2H), 7.08 (d, J = 8.2 Hz, 1H), 4.97 (s, 2H), 4.82 (br d, |
| | | J = 4.4 Hz, 2H), 4.32-4.27 (m, 1H), 4.05-3.99 (m, 1H), 3.69-3.63 (m, 1H), 3.54- |
| | | 3.49 (m, 1H), 3.26 (s, 3H), 3.24 (s, 3H), 2.65-2.63 (m, 1H), 1.17 (d, J = 7.0 Hz, |
| | | 3H), 1.14-1.09 (m, 1H), 0.78-0.77 (m, 1H) ppm |
| 669 | 574.2 | 1H NMR (400 MHz, DMSO-d6) δ = 9.63-9.62 (m, 1H), 9.39 (s, 1H), 8.66 (s, 2H), |
| | | 8.53 (d, J = 1.6 Hz, 1H), 8.28-8.27 (m, 1H), 7.89 (d, J = 7.2 Hz, 1H), 7.83 (s, 1H), |
| | | 7.78-7.67 (m, 2H), 7.09 (d, J = 8.2 Hz, 1H), 4.97 (s, 2H), 4.82 (br d, J = 5.6 Hz, |
| | | 2H), 4.29-4.27 (m, 1H), 4.02-4.00 (m, 1H), 3.70-3.61 (m, 1H), 3.54-3.50 (m, |
| | | 1H), 3.26 (s, 3H), 3.24 (s, 3H), 2.68-2.62 (m, 1H), 1.17 (d, J = 7.0 Hz, 3H), 1.14- |
| | | 1.08 (m, 1H), 0.82-0.74 (m, 1H) ppm |
| 670 | 574.2 | 1H NMR (400 MHz, DMSO-d6) δ = 9.62-9.61 (m, 1H), 9.38 (s, 1H), 8.65 (s, 2H), |
| | | 8.52 (d, J = 1.8 Hz, 1H), 8.41-8.33 (m, 1H), 8.27-8.26 (m, 1H), 7.91 (d, J = 7.4 |
| | | Hz, 1H), 7.82 (s, 1H), 7.79-7.71 (m, 2H), 7.07 (d, J = 8.4 Hz, 1H), 4.96 (s, 2H), |
| | | 4.81 (br d, J = 5.6 Hz, 2H), 4.28-4.27 (m, 1H), 4.01-3.99 (m, 1H), 3.69-3.61 (m, |
| | | 1H), 3.41-3.40 (m, 1H), 3.37 (s, 3H), 3.19 (s, 3H), 2.73-2.67 (m, 1H), 1.20-1.19 |
| | | (m, 1H), 1.16 (d, J = 7.2 Hz, 3H), 0.96-0.84 (m, 1H) ppm |
| 671 | 571.2 | 1H NMR (400 MHz, METHANOL-d4) δ = 9.36 (s, 1H), 8.64-8.59 (m, 2H), 8.21- |
| | | 8.18 (m, 1H), 7.88 (s, 1H), 7.79 (d, J = 8.4 Hz, 1H), 7.64 (d, J = 7.6 Hz, 1H), 7.40- |
| | | 7.34 (m, 2H), 7.34-7.28 (m, 1H), 5.04 (s, 2H), 4.93 (s, 2H), 4.63-4.58 (m, 2H), |
| | | 4.56-4.48 (m, 2H), 4.36-4.31 (m, 2H), 3.68-3.64 (m, 1H), 3.63 (s, 2H), 3.54- |
| | | 3.51 (m, 2H), 3.08-3.05 (m, 2H), 2.71-2.68 (m, 2H) ppm |
| 672 | 574.2 | 1H NMR (400 MHz, DMSO-d6) δ = 9.64-9.63 (m, 1H), 9.40 (s, 1H), 8.66 (s, 2H), |
| | | 8.53 (d, J = 1.6 Hz, 1H), 8.29-8.27(m, 1H), 7.93 (d, J = 7.2 Hz, 1H), 7.84 (s, 1H), |
| | | 7.80-7.73 (m, 2H), 7.09 (d, J = 8.0 Hz, 1H), 4.97 (s, 2H), 4.82 (br d, J = 5.6 Hz, |
| | | 2H), 4.30-1-4.28 (m, 1H), 4.05-4.00 (m, 1H), 3.70-3.62 (m, 1H), 3.42-3.41 (m, |
| | | 1H), 3.38 (s, 3H), 3.20 (s, 3H), 2.72-2.71 (m, 1H), 1.24-1.19 (m, 1H), 1.17 (d, J = |
| | | 7.2 Hz, 3H), 0.94-0.88 (m, 1H) ppm |
| 673 | 558.2 | 1H NMR (400 MHz, DMSO-d6) δ = 9.63-9.61 (m, 1H), 9.39 (s, 1H), 9.20 (s, 1H), |
| | | 8.64 (d, J = 8.4 Hz, 1H), 8.54 (d, J = 1.6 Hz, 1H), 8.41 (d, J = 8.8 Hz, 1H), 8.27- |
| | | 8.24 (m, 1H), 8.06-7.95 (m, 2H), 7.83 (d, J = 8.8 Hz, 1H), 7.73 (d, J = 8.0 Hz, 1H), |
| | | 7.42-7.46 (m, 1H), 4.98 (s, 2H), 4.84 (d, J = 5.6 Hz, 2H), 4.69-4.66 (m, 2H), 4.24- |
| | | 4.21 (m, 2H), 3.88-3.85 (m, 2H), 3.69-3.64 (m, 2H), 3.18 (s, 3H) ppm |
| 674 | 573.2 | 1H NMR (400 MHz, DMSO-d6) δ = 9.62-9.61 (m, 1H), 9.41 (s, 1H), 8.64 (d, J = |
| | | 8.4 Hz, 1H), 8.50 (d, J = 1.8 Hz, 1H), 8.39 (s, 2H), 8.23-8.22 (m, 1H), 7.81-7.75 |
| | | (m, 2H), 7.71 (d, J = 7.8 Hz, 1H), 7.33-7.26 (m, 2H), 7.26-7.21 (m, |
| | | 1H), 4.96 (s, 2H), 4.81 (d, J = 5.6 Hz, 2H), 4.26-4.18 (m, 2H), 3.70-3.63 (m, 4H), |
| | | 3.32-3.32 (m, 2H), 3.06 (s, 3H), 2.95-2.87 (m, 2H), 2.77-2.69 (m, 2H), 2.54- |
| | | 2.52 (m, 2H) ppm |
| 675 | 579 | 1H NMR (400 MHz, METHANOL-d4) δ = 9.39 (s, 1H), 8.67-8.59 (m, 2H), 8.22- |
| | | 8.21 (m, 1H), 7.93 (s, 1H), 7.80 (d, J = 8.6 Hz, 1H), 7.65 (d, J = 7.8 Hz, 1H), 7.40- |
| | | 7.28 (m, 3H), 6.09-5.74 (m, 1H), 5.06 (s, 2H), 4.96 (br s, 2H), 4.39-4.32 (m, 2H), |
| | | 3.86 (s, 2H), 3.57-3.51 (m, 2H), 3.08-3.03 (m, 2H), 2.99-2.95 (m, 2H), 2.85- |
| | | 2.84 (m, 2H) ppm |
| 676 | 601.3 | 1H NMR (400 MHz, DMSO-d6) δ = 9.57-9.56 (m, 1H), 8.99 (s, 1H), 8.52 (d, J = |
| | | 1.8 Hz, 1H), 8.25-5.24 (m, 1H), 8.16 (d, J = 9.2 Hz, 1H), 7.73 (d, J = 7.8 Hz, 1H), |
| | | 7.46 (d, J = 9.2 Hz, 1H), 7.39 (s, 1H), 7.15 (d, J = 8.4 Hz, 1H), 6.92 (d, |
| | | J = 2.4 Hz, 1H), 6.71-6.70 (m, 1H), 4.97 (s, 2H), 4.77-4.63 (m, 4H), 4.38-4.82 (m, |
| | | 2H), 4.27-4.19 (m, 2H), 4.13 (d, J = 6.8 Hz, 2H), 3.97-3.96 (m, 2H), 3.73-3.65 |
| | | (m, 2H), 3.46 (br s, 1H), 2.68-2.63 (m, 2H), 2.52 (br s, 1H), 1.89-1, 87 (m, 2H) ppm |
| 677 | 572.2 | 1H NMR (400 MHz, DMSO-d6) δ = 9.58-9.55 (m, 1H), 9.05 (s, 1H), 8.53 (d, J = |
| | | 1.6 Hz, 1H), 8.26-8.24 (m, 1H), 8.19-8.13 (m, 1H), 8.09 (d, J = 5.6 Hz, 1H), 7.91 |
| | | (d, J = 9.2 Hz, 1H), 7.74 (d, J = 7.9 Hz, 1H), 7.47 (s, 1H), 7.06 (d, J = 5.6 Hz, 1H), |
| | | 4.98 (s, 2H), 4.73 (d, J = 5.6 Hz, 2H), 4.23-4.22 (m, 2H), 4.16-4.09 (m, 2H), 4.00- |
| | | 3.99 (m, 1H), 3.73-3.66 (m, 2H), 2.62-2.59 (m, 2H), 1.94-1.85 (m, 2H), 0.89- |
| | | 0.81 (m, 2H), 0.76-0.68 (m, 2H) ppm |
| 678 | 537.2 | 1H NMR (400 MHz, DMSO-d6) δ = 9.66-9.65(m, 1H), 9.45 (s, 1H), 8.74 (d, J = 8.2 |
| | | Hz, 2H), 8.69-8.64 (m, 1H), 8.54 (d, J = 1.8 Hz, 1H), 8.27-8.25 (m, 1H), 8.10 (d, |
| | | J = 8.2 Hz, 1H), 7.86 (s, 1H), 7.74 (d, J = 7.8 Hz, 1H), 4.98 (s, 2H), 4.84 (d, J = 5.6 |
| | | Hz, 2H), 4.27-4.19 (m, 2H), 3.72-3.64 (m, 2H), 3.17-3.10 (m, 2H), 2.81-2.68 (m, 2H) ppm |
| 679 | 531.3 | 1H NMR (400 MHz, DMSO-d6) δ 9.18 (s, 1H), 8.56 (d, J = 1.9 Hz, 1H), 8.28 |
| | | (dd, J = 7.7, 1.9 Hz, 1H), 8.05-7.95 (m, 2H), 7.76 (d, J = 7.8 Hz, 1H), 7.64 (s, 1H), |
| | | 7.13 (d, J = 8.0 Hz, 1H), 6.71 (d, J = 8.0 Hz, 1H), 5.79 (s, 5H), 5.01 (s, 2H), 4.76 |
| | | (d, J = 5.8 Hz, 2H), 4.35 (t, J = 4.4 Hz, 2H), 4.30-4.23 (m, 2H), 4.05-3.98 (m, |
| | | 2H), 3.73-3.66 (m, 2H), 2.25 (s, 3H). |
| 680 | 545.3 | 1H NMR (400 MHz, DMSO-d6) δ 9.56 (t, J = 5.9 Hz, 1H), 9.18 (s, 1H), 8.55 (d, J = |
| | | 1.9 Hz, 1H), 8.30 (dd, J = 7.8, 1.9 Hz, 1H), 8.05-7.94 (m, 2H), 7.76 (d, J = 7.8 Hz, |
| | | 1H), 7.71 (s, 1H), 7.65 (s, 1H), 7.13 (d, J = 7.9 Hz, 1H), 6.71 (d, J = 8.0 Hz, 1H), |
| | | 4.99 (s, 2H), 4.76 (d, J = 5.7 Hz, 2H), 4.38-4.29 (m, 3H), 4.10-3.98 (m, 3H), 3.70- |
| | | 3.63 (m, 1H), 2.25 (s, 3H), 1.20 (d, J = 7.0 Hz, 3H). |
| 681 | 545.3 | 1H NMR (400 MHz, METHANOL-d4) δ = 9.01 (s, 1H), 8.61 (d, J = 2.0 Hz, 1H), 8.44 |
| | | (d, J = 1.6 Hz, 1H), 8.21-8.19 (m, 1H), 8.16 (d, J = 8.8 Hz, 1H), 7.96 (d, J = 9.2 |
| | | Hz, 1H), 7.75 (d, J = 5.2 Hz, 1H), 7.66-7.63 (m, 2H), 6.95 (d, J = 4.8 Hz, 1H), 5.05 |
| | | (s, 2H), 4.85 (s, 2H), 4.36-4.33 (m, 2H), 4.31-4.25 (m, 2H), 3.54-3.51 (m, 2H), |
| | | 3.30-3.27 (m, 2H), 2.83 (s, 3H), 2.39 (s, 3H) ppm |
| 682 | 581.2 | 1H NMR (400 MHz, DMSO-d6) δ = 9.58-9.56 (m, 1H), 9.07 (s, 1H), 8.51 (d, J = |
| | | 1.6 Hz, 1H), 8.33 (d, J = 9.2 Hz, 1H), 8.26-8.22 (m, 1H), 7.76-7.69 (m, 2H), 7.53 |
| | | (d, J = 9.2 Hz, 1H), 7.40 (s, 1H), 7.23-7.21 (m, 1H), 7.04 (d, J = 8.4 Hz, 1H), 4.97 |
| | | (s, 2H), 4.71 (d, J = 5.6 Hz, 2H), 4.36-4.30 (m, 2H), 4.24-4.20 (m, 4H), 3.70- |
| | | 3.65 (m, 2H), 1.95-1.86 (m, 3H) ppm |
| 683 | 586.1 | 1H NMR (400 MHz, DMSO-d6) δ = 9.59-9.56 (m, 1H), 9.05 (s, 1H), 8.52 (d, J = |
| | | 2.0 Hz, 1H), 8.25-8.23 (m, 7.9 Hz, 1H), 8.15 (d, J = 9.6 Hz, 1H), 7.77-7.68 (m, |
| | | 2H), 7.48 (s, 1H), 7.28 (s, 1H), 4.98 (s, 2H), 4.73 (d, J = 5.2 Hz, 2H), 4.27-4.17 (m, |
| | | 2H), 4.13-4.07 (m, 2H), 3.84 (s, 3H), 3.72-3.64 (m, 2H), 2.80-2.77 (m, 2H), 2.37- |
| | | 2.33 (m, 1H), 1.94-1.88 (m, 2H), 0.90-0.74 (m, 4H) ppm |
| 684 | 574.2 | 1H NMR (400 MHz, DMSO-d6) δ = 9.56-9.46 (m, 1H), 9.39 (s, 1H), 8.70-8.58 (m, |
| | | 2H), 8.49-8.43 (m, 1H), 8.17-8.15 (m, 1H), 7.91 (d, J = 7.4 Hz, 1H), 7.81-7.71 |
| | | (m, 2H), 7.28-7.20 (m, 1H), 7.03 (d, J = 8.4 Hz, 1H), 4.89-4.75 (m, 3H), 4.67- |
| | | 4.57 (m, 1H), 4.32 (br d, J = 11.4 Hz, 2H), 4.03-3.90 (m, 1H), 3.74-3.61 (m, 2H), |
| | | 2.52 (br s, 2H), 1.34 (d, J = 7.0 Hz, 3H), 1.21 (d, J = 6.4 Hz, 6H) ppm |
| 685 | 555.3 | 1H NMR (400 MHz, DMSO-d6) δ = 9.64-9.61 (m, 1H), 9.43 (s, 1H), 8.66-8.64 (m, |
| | | 1H), 8.50 (d, J = 2.0 Hz, 1H), 8.24-8.22 (m, 1H), 8.16 (d, J = 1.6 Hz, 1H), 7.83- |
| | | 7.76 (m, 2H), 7.72 (d, J = 8.0 Hz, 1H), 7.32-7.27 (m, 2H), 7.26-7.22 (m, 1H), 4.96 |
| | | (s, 2H), 4.82 (d, J = 5.6 Hz, 2H), 4.26-4.17 (m, 2H), 3.74 (s, 2H), 3.70-3.61 (m, |
| | | 2H), 2.94-2.84 (m, 4H), 1.72-1.69 (m, 1H), 0.38-0.30 (m, 2H), 0.28-0.17 (m, 2H) ppm |
| 686 | 557.3 | 1H NMR (400 MHz, DMSO-d6) δ = 9.61-9.50 (m, 1H), 9.04 (s, 1H), 8.53 (d, J = |
| | | 1.6 Hz, 1H), 8.25 (d, J = 7.6 Hz, 1H), 8.18 (d, J = 9.2 Hz, 1H), 8.01 (d, J = 9.2 Hz, |
| | | 1H), 7.73 (d, J = 7.6 Hz, 1H), 7.66 (d, J = 1.6 Hz, 1H), 7.50-7.40 (m, 2H), 7.04 (d, |
| | | J = 8.0 Hz, 1H), 4.98 (s, 2H), 4.72 (d, J = 6.0 Hz, 2H), 4.23 (d, J = 4.8 Hz, 4H), 3.76- |
| | | 3.64 (m, 2H), 3.42-3.39 (m, 2H), 2.46-2.43 (m, 1H), 0.91-0.81 (m, 2H), 0.66- |
| | | 0.56 (m, 2H) ppm |
| 687 | 574 | 1H NMR (400 MHz, DMSO-d6) δ = 9.56-9.45 (m, 1H), 9.39 (s, 1H), 8.65-8.61 (m, |
| | | 2H), 8.48 (d, J = 0.8 Hz, 1H), 8.46 (d, J = 2.0 Hz, 1H), 8.17 (d, J = 8.8 Hz, 1H), 7.91 |
| | | (d, J = 7.6 Hz, 1H), 7.79 (s, 1H), 7.74 (d, J = 8.0 Hz, 1H), 7.24 (d, J = 8.8 Hz, 1H), |
| | | 7.03 (d, J = 8.8 Hz, 1H), 4.86-4.75 (m, 3H), 4.62 (d, J = 8.4 Hz, 1H), 4.31 (d, J = |
| | | 11.6 Hz, 2H), 4.04-3.92 (m, 1H), 3.72-3.63 (m, 2H), 2.56-2.54 (m, 2H), 1.34 (d, |
| | | J = 7.2 Hz, 3H), 1.21 (d, J = 6.0 Hz, 6H) ppm |
| 688 | 519.2 | 1H NMR (400 MHz, DMSO-d6) δ = 9.55-9.49 (m, 1H), 8.90 (s, 1H), 8.60 (d, J = |
| | | 9.2 Hz, 1H), 8.52 (d, J = 1.8 Hz, 1H), 8.24-8.23 (m, 1H), 8.13 (d, J = 8.8 Hz, 1H), |
| | | 7.73 (d, J = 7.8 Hz, 1H), 7.49 (s, 1H), 7.31 (s, 1H), 4.97 (s, 2H), 4.67 (d, J = 5.4 Hz, |
| | | 2H), 4.26-4.13 (m, 4H), 3.67-3.65 (m, 2H), 3.57 (s, 3H), 2.66-2.64 (m, 2H), 2.00- |
| | | 1.92 (m, 2H) ppm |
| 689 | 592.1 | 1H NMR (400 MHz, METHANOL-d4) δ = 9.00 (s, 1H), 8.61 (d, J = 1.8 Hz, 1H), 8.21- |
| | | 8.20 (m, 1H), 8.09 (d, J = 9.2 Hz, 1H), 7.88 (d, J = 9.2 Hz, 1H), 7.68-7.60 (m, |
| | | 2H), 7.52 (d, J = 7.6 Hz, 1H), 7.03 (d, J = 7.6 Hz, 1H), 5.04 (s, 2H), 4.85 (s, 2H), |
| | | 4.37-4.30 (m, 3H), 4.09-4.07 (m, 1H), 3.52-3.51 (m, 2H), 2.95-2.84 (m, 3H), |
| | | 2.11-2.00 (m, 3H), 1.79-1.67 (m, 1H) ppm |
| 690 | 592.1 | 1H NMR (400 MHz, METHANOL-d4) δ = 9.00 (s, 1H), 8.61 (d, J = 1.8 Hz, 1H), 8.20- |
| | | 8.19 (m, 1H), 8.10 (d, J = 9.2 Hz, 1H), 7.88 (d, J = 9.2 Hz, 1H), 7.68-7.61 (m, |
| | | 2H), 7.52 (d, J = 7.8 Hz, 1H), 7.03 (d, J = 7.6 Hz, 1H), 5.04 (s, 2H), 4.84 (s, 2H), |
| | | 4.39-4.29 (m, 3H), 4.09-4.07 (m, 1H), 3.55-3.49 (m, 2H), 2.96-2.82 (m, 3H), |
| | | 2.11-2.02 (m, 3H), 1.77-1.67 (m, 1H) ppm |
| 691 | 569.8 | 1H NMR (400 MHz, DMSO-d6) δ = 9.65-9.64 (m, 1H), 9.13 (s, 1H), 8.40 (d, J = |
| | | 1.4 Hz, 1H), 8.33 (d, J = 9.4 Hz, 1H), 8.23 (d, J = 9.2 Hz, 1H), 8.16-8.15 (m, 1H), |
| | | 7.98-7.93 (m, 1H), 7.56-7.52 (m, 2H), 5.02 (s, 2H), 4.74 (br d, J = 5.4 Hz, 2H), |
| | | 4.41-4.32 (m, 4H), 4.27-4.19 (m, 2H), 3.81-3.72 (m, 2H) ppm |
| 692 | 586.9 | 1H NMR (400 MHz, DMSO-d6) δ = 9.61-9.49 (m, 1H), 9.00 (s, 1H), 8.52 (d, J = |
| | | 1.6 Hz, 1H), 8.24 (dd, J = 2.0, 7.9 Hz, 1H), 8.17 (d, J = 9.2 Hz, 1H), 7.76-7.69 (m, |
| | | 1H), 7.46-7.37 (m, 2H), 7.14 (d, J = 8.0 Hz, 1H), 6.74 (d, J = 2.3 Hz, 1H), 6.53- |
| | | 6.52 (m, 1H), 5.20-5.19 (m, 1H), 5.01-4.94 (m, 2H), 4.83-4.82 (m, 2H), 4.70 (br |
| | | d, J = 5.6 Hz, 2H), 4.53-4.52 (m, 2H), 4.28-4.18 (m, 2H), 3.97-3.96 (m, 2H), |
| | | 3.72-3.64 (m, 2H), 2.66-2.64 (m, 2H), 1.93-1.85 (m, 2H) ppm |
| 693 | 417.9 | 1H NMR (400 MHz, DMSO-d6) δ = 9.63-9.61 (m, 1H), 9.42 (s, 1H), 8.64 (d, J = |
| | | 8.4 Hz, 1H), 8.49 (s, 1H), 8.24-8.21 (m, 1H), 7.74-7.71 (m, 3H), 4.98 (s, 2H), 4.79 |
| | | (br d, J = 5.6 Hz, 2H), 4.23-4.21 (m, 2H), 3.68-3.65 (m, 2H) ppm |
| 694 | 544.3 | 1H NMR (400 MHz, DMSO-d6) δ = 9.62-9.61 (m, 1H), 9.36 (s, 1H), 8.62 (s, 2H), |
| | | 8.54 (d, J = 1.6 Hz, 1H), 8.33 (br s, 1H), 8.26-8.24 (m, 1H), 7.83-7.69 (m, 3H), |
| | | 7.43 (d, J = 8.2 Hz, 1H), 4.98 (s, 2H), 4.81 (br d, J = 5.6 Hz, 2H), 4.30-4.18 (m, |
| | | 2H), 3.72-3.65 (m, 2H), 3.43 (br s, 2H), 3.21 (s, 3H), 2.99-2.88 (m, 1H), 1.97- |
| | | 1.95 (m, 1H), 1.72-1.59 (m, 1H), 1.25 (d, J = 6.8 Hz, 3H) ppm |
| 695 | 544.3 | 1H NMR (400 MHz, DMSO-d6) δ = 9.62-9.61 (m, 1H), 9.36 (s, 1H), 8.62 (s, 2H), |
| | | 8.54 (d, J = 1.6 Hz, 1H), 8.33 (br s, 1H), 8.26-8.24 (m, 1H), 7.83-7.69 (m, 3H), |
| | | 7.43 (d, J = 8.2 Hz, 1H), 4.98 (s, 2H), 4.81 (br d, J = 5.6 Hz, 2H), 4.30-4.18 (m, |
| | | 2H), 3.72-3.65 (m, 2H), 3.43 (br s, 2H), 3.21 (s, 3H), 2.99-2.88 (m, 1H), 1.97- |
| | | 1.95 (m, 1H), 1.72-1.59 (m, 1H), 1.25 (d, J = 6.8 Hz, 3H) ppm |
| 696 | 603.2 | 1H NMR (400 MHz, DMSO-d6) δ = 9.59-9.51 (m, 1H), 8.99 (s, 1H), 8.52 (d, J = |
| | | 1.6 Hz, 1H), 8.53-8.52 (m, 1H), 8.24 (d, J = 1.6 Hz, 1H), 8.17 (d, J = 9.2 Hz, 1H), |
| | | 7.73 (d, J = 7.6 Hz, 1H), 7.47-7.33 (m, 2H), 7.11 (d, J = 8.8 Hz, 1H), 6.92 (d, J = |
| | | 2.4 Hz, 1H), 6.67 (d, J = 2.0 Hz, 1H), 4.98 (s, 2H), 4.70 (d, J = 6.0 Hz, 2H), 4.51- |
| | | 4.42 (m, 1H), 4.26-4.19 (m, 2H), 3.97-3.95(m, 2H), 3.70-3.65 (m, 2H), 3.43 (d, |
| | | J = 6.0 Hz, 2H), 3.25 (s, 3H), 2.67-2.63 (m, 2H), 1.94-1.85 (m, 2H), 1.16 (d, J = 6.0 |
| | | Hz, 3H) ppm |
| 697 | 603.2 | 1H NMR (400 MHz, DMSO-d6) δ = 9.59-9.50 (m, 1H), 8.99 (s, 1H), 8.52 (d, J = |
| | | 1.6 Hz, 1H), 8.43-8.34 (m, 1H), 8.24 (d, J = 1.6 Hz, 1H), 8.17 (d, J = 9.2 Hz, 1H), |
| | | 7.73 (d, J = 8.0 Hz, 1H), 7.42 (d, J = 9.2 Hz, 1H), 7.37 (s, 1H), 7.11 (d, J = 8.4 Hz, |
| | | 1H), 6.91 (d, J = 2.4 Hz, 1H), 6.67 (d, J = 2.4 Hz, 1H), 4.97 (s, 2H), 4.70 (d, J = 5.6 |
| | | Hz, 2H), 4.47 (d, J = 6.0 Hz, 1H), 4.26-4.19 (m, 2H), 3.97 (d, J = 6.4 Hz, 2H), 3.71- |
| | | 3.64 (m, 2H), 3.42 (d, J = 6.0 Hz, 1H), 3.39 (d, J = 4.0 Hz, 1H), 3.25 (s, 3H), 2.66- |
| | | 2.63 (m, 2H), 1.92-1.87 (m, 2H), 1.16 (d, J = 6.4 Hz, 3H) ppm |
| 698 | 592.1 | 1H NMR (400 MHz, DMSO-d6) δ = 9.67-9.51 (m, 1H), 9.13 (s, 1H), 8.52 (d, J = |
| | | 2.0 Hz, 1H), 8.34 (d, J = 9.2 Hz, 1H), 8.32 (s, 1H), 8.25-8.23 (m, 1H), 8.08 (d, J = |
| | | 9.2 Hz, 1H), 7.73 (d, J = 7.6 Hz, 1H), 7.50 (s, 1H), 7.44 (s, 1H), 4.97 (s, 2H), 4.74 |
| | | (d, J = 6.0 Hz, 2H), 4.35-4.20 (m, 6H), 3.71-3.65 (m, 2H), 2.37-2.34 (m, 1H), |
| | | 0.97-0.91 (m, 2H), 0.87-0.81 (m, 2H) ppm |
| 699 | 570.1 | 1H NMR (400 MHz, METHANOL-d4) δ = 9.35 (s, 1H), 8.75-8.70 (m, 1H), 8.68- |
| | | 8.60 (m, 2H), 8.57-8.51 (m, 1H), 8.45 (d, J = 7.6 Hz, 1H), 8.24-8.23 (m, 1H), 8.00 |
| | | (s, 1H), 7.94-7.93 (m, 1H), 7.67 (d, J = 7.8 Hz, 1H), 7.54 (d, J = 7.8 Hz, 1H), 5.07 |
| | | (s, 2H), 4.95 (s, 2H), 4.43-4.33 (m, 2H), 3.59-3.52 (m, 3H), 3.07-2.92 (m, 3H), |
| | | 2.79-2.70 (m, 1H), 2.61 (s, 3H), 2.45-2.43 (m, 1H), 2.27-2.11 (m, 1H), 1.57- |
| | | 1.55 (m, 1H), 1.21-1.19 (m, 1H) ppm |
| 700 | 594.1 | 1H NMR (400 MHz, METHANOL-d4) δ = 9.03 (s, 1H), 8.61 (d, J = 1.8 Hz, 1H), 8.30- |
| | | 8.24 (m, 1H), 8.23-8.17 (m, 2H), 7.69-7.57 (m, 2H), 7.25 (d, J = 8.2 Hz, 1H), |
| | | 7.04 (d, J = 8.2 Hz, 1H), 5.04 (s, 2H), 4.85 (br s, 2H), 4.65-4.60 (m, 1H), 4.39- |
| | | 4.37 (m, 1H), 4.36-4.27 (m, 3H), 4.19-4.10 (m, 1H), 3.52-3.50 (m, 2H), 2.97- |
| | | 2.85 (m, 1H), 2.10-2.01 (m, 1H), 1.84-1.72 (m, 1H) ppm |
| 701 | 558.2 | 1H NMR (400 MHz, DMSO-d6) δ = 9.65-9.63 (m, 1H), 9.37 (s, 1H), 9.18 (d, J = |
| | | 8.8 Hz, 1H), 8.68 (d, J = 8.8 Hz, 1H), 8.59 (s, 1H), 8.55 (d, J = 1.6 Hz, 1H), 8.45- |
| | | 8.40 (m, 1H), 8.28-8.26 (m, 1H), 7.94 (d, J = 8.0 Hz, 1H), 7.82 (s, 1H), 7.74 (d, J = |
| | | 8.0 Hz, 1H), 7.29-7.22 (m, 1H), 4.98 (s, 2H), 4.83 (d, J = 5.6 Hz, 2H), 4.72-4.69 |
| | | (m, 2H), 4.27-4.19 (m, 2H), 3.91-3.89 (m, 2H), 3.71-3.66 (m, 2H), 3.26 (s, 3H) ppm |
| 702 | 594.1 | 1H NMR (400 MHz, METHANOL-d4) δ = 9.02 (s, 1H), 8.61 (d, J = 1.6 Hz, 1H), 8.31- |
| | | 8.24 (m, 1H), 8.24-8.17 (m, 2H), 7.69-7.60 (m, 2H), 7.26 (d, J = 8.2 Hz, 1H), |
| | | 7.04 (d, J = 8.2 Hz, 1H), 5.05 (s, 2H), 4.94 (br d, J = 1.8 Hz, 2H), 4.65-4.60 (m, |
| | | 1H), 4.44-4.38 (m, 1H), 4.36-4.28 (m, 3H), 4.22-4.20 (m, 1H), 3.55-3.51 (m, |
| | | 2H), 2.91-2.90 (m, 1H), 2.17-1.96 (m, 1H), 1.89-1.67 (m, 1H) ppm |
| 703 | 532.2 | 1H NMR (400 MHz, DMSO-d6) δ = 9.60-9.52 (m, 1H), 9.11-9.02 (m, 2H), 8.57- |
| | | 8.50 (m, 1H), 8.48-8.44 (m, 1H), 8.39-8.31 (m, 1H), 8.29-8.22 (m, 1H), 7.85 (d, |
| | | J = 2.4 Hz, 1H), 7.77-7.71 (m, 1H), 7.48-7.44 (m, 1H), 7.41 (br s, 1H), 4.98 (s, |
| | | 2H), 4.72 (br d, J = 5.8 Hz, 2H), 4.37-4.30 (m, 2H), 4.26-4.19 (m, 2H), 3.80 (s, |
| | | 3H), 3.70-3.66 (m, 2H), 3.17-3.13 (m, 2H) ppm |
| 704 | 600.3 | 1H NMR (400 MHz, DMSO-d6) δ = 9.60-9.53 (m, 1H), 9.39 (s, 1H), 8.69-8.59 (m, |
| | | 2H), 8.50 (d, J = 2.0 Hz, 1H), 8.44 (s, 1H), 8.24-8.22 (m, 1H), 7.91 (d, J = 7.2 Hz, |
| | | 1H), 7.80 (s, 1H), 7.77-7.71 (m, 1H), 7.19 (d, J = 8.4 Hz, 1H), 7.03 (d, J = 8.4 Hz, |
| | | 1H), 4.80 (d, J = 5.6 Hz, 2H), 4.31 (d, J = 11.6 Hz, 2H), 3.70-3.65 (m, 2H), 3.64- |
| | | 3.61 (m, 2H), 2.53 (s, 2H), 2.32 (d, J = 2.0 Hz, 2H), 1.21 (d, J = 6.0 Hz, 6H), 0.98- |
| | | 0.92 (m, 2H), 0.76-0.70 (m, 2H) ppm |
| 705 | 603.3 | 1H NMR (400 MHz, DMSO-d6) δ = 9.56-9.54 (m, 1H), 8.98 (s, 1H), 8.51 (d, J = |
| | | 2.0 Hz, 1H), 8.43 (s, 1H), 8.24 (d, J = 2.0 Hz, 1H), 8.15 (d, J = 9.2 Hz, 1H), 7.72 (d, |
| | | J = 7.6 Hz, 1H), 7.44 (d, J = 9.2 Hz, 1H), 7.38 (s, 1H), 7.12 (d, J = 8.4 Hz, 1H), 6.90 |
| | | (d, J = 2.4 Hz, 1H), 6.68 (d, J = 8.4 Hz, 1H), 4.97 (s, 2H), 4.69 (d, J = 6.0 Hz, 2H), |
| | | 4.27-4.18 (m, 2H), 4.00-3.93 (m, 2H), 3.87-3.82 (m, 2H), 3.69-3.64 (m, 2H), |
| | | 3.61 (d, J = 6.0 Hz, 1H), 3.26 (s, 3H), 2.67-2.63 (m, 2H), 1.92-1.85 (m, 2H), 1.11 |
| | | (d, J = 6.4 Hz, 3H) ppm |
| 706 | 603.3 | 1H NMR (400 MHz, DMSO-d6) δ = 9.54-9.53 (m, 1H), 8.99 (s, 1H), 8.52 (d, J = |
| | | 1.8 Hz, 1H), 8.37-8.28 (m, 1H), 8.24-8.23 (m, 1H), 8.16 (d, J = 9.2 Hz, 1H), 7.73 |
| | | (d, J = 7.8 Hz, 1H), 7.45 (d, J = 9.2 Hz, 1H), 7.39 (s, 1H), 7.13 (d, J = |
| | | 8.4 Hz, 1H), 6.91 (d, J = 2.4 Hz, 1H), 6.69-6.68 (m, 1H), 4.98 (s, 2H), 4.70 (d, J = |
| | | 5.8 Hz, 2H), 4.25-4.20 (m, 2H), 3.97-3.95 (m, 2H), 3.87-3.84 (m, 2H), 3.71- |
| | | 3.65 (m, 2H), 3.60 (s, 1H), 3.27 (s, 3H), 2.71-2.65 (m, 2H), 1.89-1.87 (m, 2H), |
| | | 1.12 (d, J = 6.4 Hz, 3H) ppm |
| 707 | 574.3 | 1H NMR (400 MHz, DMSO-d6) δ 8.52 (s, 1H), 8.05 (s, 1H), 7.86-7.75 (m, 2H), |
| | | 7.56 (d, J = 6.2 Hz, 1H), 7.47-7.36 (m, 2H), 7.16 (s, 1H), 6.86 (d, J = 7.9 Hz, 1H), |
| | | 6.01 (d, J = 6.3 Hz, 1H), 4.27 (s, 2H), 4.13 (s, 2H), 3.60-3.52 (m, 2H), 2.98-2.94 |
| | | (m, 1H), 2.75 (d, J = 5.3 Hz, 2H), 2.08-1.88 (m, 3H), 0.65-0.41 (m, 6H). |
| 708 | 588.4 | 1H NMR (400 MHz, Methanol-d4) δ 9.28 (s, 1H), 8.81 (d, J = 1.3 Hz, 1H), 8.59 |
| | | (d, J = 1.9 Hz, 1H), 8.55 (dd, J = 8.6, 1.6 Hz, 1H), 8.32 (d, J = 6.3 Hz, 1H), 8.20 |
| | | (dd, J = 7.8, 1.9 Hz, 1H), 8.17 (d, J = 8.7 Hz, 1H), 7.93 (s, 1H), 7.62 (d, J = 7.8 Hz, |
| | | 1H), 6.77 (d, J = 6.3 Hz, 1H), 5.08-4.95 (m, 2H), 4.89 (s, 2H), 4.49 (s, 2H), 4.42- |
| | | 4.34 (m, 1H), 4.09 (dd, J = 13.3, 5.8 Hz, 1H), 3.70 (ddd, J = 10.5, 6.2, 2.7 Hz, 1H), |
| | | 2.73-2.60 (m, 3H), 1.28-1.20 (m, 9H). |
| 709 | 586.3 | 1H NMR (400 MHz, Methanol-d4) δ 9.29 (s, 1H), 8.69 (d, J = 8.7 Hz, 1H), 8.64- |
| | | 8.53 (m, 1H), 8.42 (s, 3H), 8.22 (dd, J = 7.8, 1.9 Hz, 1H), 7.96 (s, 1H), 7.85 (d, J = |
| | | 7.4 Hz, 1H), 7.72-7.60 (m, 2H), 6.67 (d, J = 8.3 Hz, 1H), 5.12 (s, 1H), 5.09-4.96 |
| | | (m, 2H), 4.92 (s, 2H), 4.38 (dd, J = 13.3, 2.4 Hz, 1H), 4.09 (dd, J = 13.3, 5.9 Hz, |
| | | 1H), 4.00 (dd, J = 8.8, 6.7 Hz, 1H), 3.80-3.66 (m, 3H), 3.59-3.52 (m, 3H), 3.15 |
| | | (d, J = 4.0 Hz, 1H), 2.15 (s, 3H), 1.60 (s, 1H), 1.37 (dd, J = 7.0, 2.0 Hz, 2H). |
| 710 | 589.4 | 1H NMR (400 MHz, DMSO-d6) δ 10.00 (s, 1H), 9.69 (t, J = 5.8 Hz, 1H), 9.54 (s, |
| | | 1H), 8.56 (d, J = 1.9 Hz, 1H), 8.31 (dd, J = 7.9, 1.9 Hz, 1H), 7.97 (s, 1H), 7.91 |
| | | (d, J = 7.3 Hz, 1H), 7.87-7.75 (m, 2H), 7.14 (d, J = 8.4 Hz, 1H), 5.01 (s, 2H), 4.90 |
| | | (d, J = 5.8 Hz, 2H), 4.36 (dd, J = 20.9, 11.8 Hz, 2H), 4.06 (dd, J = 13.4, 5.9 Hz, 1H), |
| | | 3.74-3.66 (m, 5H), 3.03 (s, 1H), 3.01 (s, 7H), 1.26 (d, J = 6.2 Hz, 6H), 1.21 (d, J = |
| | | 7.1 Hz, 3H). |
| 711 | 577.2 | 1H NMR (400 MHz, DMSO-d6) δ = 9.59-9.49 (m, 1H), 9.01 (s, 1H), 8.51 (d, J = |
| | | 1.6 Hz, 1H), 8.47 (s, 1H), 8.29-8.16 (m, 2H), 7.72 (d, J = 8.0 Hz, 1H), 7.41-7.32 |
| | | (m, 1H), 7.25-7.15 (m, 1H), 7.13-7.06 (m, 1H), 6.98 (d, J = 9.2 Hz, 1H), 4.97 (s, |
| | | 2H), 4.70 (d, J = 5.2 Hz, 2H), 4.43 (s, 2H), 4.28-4.17 (m, 2H), 4.15-3.89 (m, 2H), |
| | | 3.71-3.63 (m, 2H), 3.27-3.24 (m, 3H), 2.78-2.75 (m, 2H), 1.97-1.84 (m, 2H) ppm |
| 712 | 572.2 | 1H NMR (400 MHz, DMSO-d6) δ = 9.58-9.57 (m, 1H), 9.12-9.06 (m, 1H), 8.52 |
| | | (d, J = 1.6 Hz, 1H), 8.32-8.23 (m, 3H), 7.84 (d, J = 2.4 Hz, 1H), 7.73 (d, J = 7.6 |
| | | Hz, 1H), 7.52-7.43 (m, 1H), 7.15 (d, J = 2.0 Hz, 1H), 4.98 (s, 2H), 4.73 (d, J = 5.6 |
| | | Hz, 2H), 4.34-4.30 (m, 4H), 4.25-4.20 (m, 2H), 3.70-3.66 (m, 2H), 1.39 (s, 3H), |
| | | 0.93-0.82 (m, 2H), 0.80-0.70 (m, 2H) ppm |
| 713 | 572.2 | 1H NMR (400 MHz, DMSO-d6) δ = 9.60-9.57 (m, 1H), 9.11 (s, 1H), 8.53 (d, J = |
| | | 1.6 Hz, 1H), 8.38 (s, 1H), 8.33-8.29 (m, 1H), 8.28-8.21 (m, 2H), 7.74 (d, J = 8.0 |
| | | Hz, 1H), 7.50 (s, 1H), 7.25 (d, J = 8.0 Hz, 1H), 6.99 (d, J = 8.0 Hz, 1H), 4.98 (s, |
| | | 2H), 4.74 (d, J = 5.6 Hz, 2H), 4.34-4.26 (m, 4H), 4.26-4.21 (m, 2H), 3.71-3.66 |
| | | (m, 2H), 1.44 (s, 3H), 1.07-1.01 (m, 2H), 0.76-0.70 (m, 2H) ppm |
| 714 | 5563.3 | 1H NMR (400 MHz, DMSO-d6) δ = 9.55-9.54 (m, 1H), 9.00 (s, 1H), 8.51 (d, J = |
| | | 1.6 Hz, 1H), 8.24-8.23 (m, 1H), 8.16 (d, J = 9.2 Hz, 1H), 7.73 (d, J = 7.8 Hz, 1H), |
| | | 7.45 (d, J = 9.2 Hz, 1H), 7.43-7.36 (m, 1H), 7.16 (d, J = 7.8 Hz, 1H), |
| | | 7.11 (d, J = 11.6 Hz, 1H), 4.98 (s, 2H), 4.70 (br d, J = 5.6 Hz, 2H), 4.27-4.18 (m, |
| | | 2H), 3.99-3.97 (m, 2H), 3.74 (s, 3H), 3.69-3.66 (m, 2H), 2.68-2.63 (m, 2H), 1.89- |
| | | 1.87 (m, 2H) ppm |
| 715 | 592.2 | 1H NMR (400 MHz, DMSO-d6) δ = 9.63-9.62 (m, 1H), 9.40 (s, 1H), 8.72-8.60 (m, |
| | | 2H), 8.52 (d, J = 1.6 Hz, 1H), 8.44 (s, 1H), 8.28-8.27 (m, 1H), 7.91 (d, J = 7.2 Hz, |
| | | 1H), 7.83 (s, 1H), 7.78-7.70 (m, 2H), 7.03 (d, J = 8.6 Hz, 1H), 4.82 (br d, J = 5.6 |
| | | Hz, 2H), 4.31 (br d, J = 12.0 Hz, 2H), 3.73-3.60 (m, 3H), 2.54-2.51 (m, 2H), 1.21 |
| | | (d, J = 6.2 Hz, 6H), 1.16 (d, J = 7.0 Hz, 3H) ppm |
| 716 | 561.2 | 1H NMR (400 MHz, DMSO-d6) δ = 9.55-9.54 (m, 1H), 9.04 (s, 1H), 8.52 (d, J = |
| | | 1.8 Hz, 1H), 8.29 (s, 1H), 8.28-8.22 (m, 2H), 7.73 (d, J = 7.8 Hz, 1H), 7.54 (d, J = |
| | | 9.2 Hz, 1H), 7.46-7.44 (m, 1H), 7.42 (s, 1H), 7.11-7.03 (m, 1H), 7.01-6.96 (m, |
| | | 1H), 6.95-6.88 (m, 1H), 4.97 (s, 2H), 4.71 (d, J = 6.0 Hz, 2H), 4.63-4.62 (m, 1H), |
| | | 4.45-4.37 (m, 1H), 4.27-4.19 (m, 2H), 3.73 (dd, J = 7.7, 13.6 Hz, 1H), 3.70-3.65 |
| | | (m, 2H), 3.59-3.48 (m, 2H), 3.21 (s, 3H) ppm |
| 717 | 592.2 | 1H NMR (400 MHz, DMSO-d6) δ = 9.63-9.62 (m, 1H), 9.40 (s, 1H), 8.73-8.58 (m, |
| | | 2H), 8.52 (d, J = 1.6 Hz, 1H), 8.42 (s, 1H), 8.28-8.27 (m, 1H), 7.91 (d, J = 7.6 Hz, |
| | | 1H), 7.83 (s, 1H), 7.79-7.65 (m, 2H), 7.03 (d, J = 8.6 Hz, 1H), 4.82 (br d, J = 5.6 |
| | | Hz, 2H), 4.31 (br d, J = 11.6 Hz, 2H), 3.74-3.57 (m, 3H), 2.56-2.52 (m, 2H), 1.21 |
| | | (d, J = 6.2 Hz, 6H), 1.16 (d, J = 7.2 Hz, 3H) ppm |
| 718 | 563.1 | 1H NMR (400 MHz, DMSO-d6) δ = 9.54-9.53 (m, 1H), 9.02 (s, 1H), 8.51 (d, J = |
| | | 1.8 Hz, 1H), 8.26-8.19 (m, 2H), 7.72 (d, J = 7.8 Hz, 1H), 7.37 (s, 1H), 7.10-7.05 |
| | | (m, 1H), 7.01-7.00 (m, 1H), 6.81-6.80 (m, 1H), 4.97 (s, 2H), 4.70 (d, J = 5.8 Hz, |
| | | 2H), 4.22-4.20 (m, 2H), 3.81 (s, 3H), 3.72-3.64 (m, 2H), 3.30 (s, 2H), 2.67-2.65 |
| | | (m, 2H), 1.84-1.82 (m, 2H) |
| 719 | 546.1 | 1H NMR (400 MHz, DMSO-d6) δ = 9.54-9.53 (m, 1H), 8.98 (s, 1H), 8.56-8.48 (m, 1H), |
| | | 8.46 (br s, 1H), 8.33-8.08 (m, 3H), 7.72 (d, J = 7.8 Hz, 1H), 7.49-7.30 (m, |
| | | 2H), 6.81-6.66 (m, 1H), 4.97 (s, 2H), 4.69 (br d, J = 6.0 Hz, 2H), 4.30-4.17 (m, |
| | | 2H), 3.98-3.96 (m, 2H), 3.84 (s, 3H), 3.70-3.64 (m, 2H), 2.72-2.70 (m, 2H), 1.96- |
| | | 1.84 (m, 2H) ppm |
| 720 | 552.3 | 1H NMR (400 MHz, DMSO-d6) δ = 9.62-9.55 (m, 1H), 9.15-9.09 (m, 2H), 8.53 |
| | | (d, J = 1.6 Hz, 1H), 8.47 (s, 1H), 8.45 (d, J = 9.4 Hz, 1H), 8.34 (s, 1H), 8.25-8.23 |
| | | (m, 1H), 7.82 (s, 1H), 7.73 (d, J = 7.8 Hz, 1H), 7.52 (s, 1H), 7.22-6.91 (m, 1H), |
| | | 4.98 (s, 2H), 4.75 (d, J = 5.8 Hz, 2H), 4.40-4.38 (m, 2H), 4.25-4.20 (m, 2H), 3.70- |
| | | 3.66 (m, 2H), 3.22 (br d, J = 8.2 Hz, 2H) ppm |
| 721 | 565 | 1H NMR (400 MHz, DMSO-d6) δ = 9.87-9.73 (m, 1H), 9.48 (d, J = 1.6 Hz, 1H), |
| | | 9.07 (s, 1H), 8.89 (s, 1H), 8.18 (d, J = 9.2 Hz, 1H), 7.83 (d, J = 9.2 Hz, 1H), 7.78- |
| | | 7.41 (m, 3H), 6.97 (d, J = 7.6 Hz, 1H), 4.78 (d, J = 5.6 Hz, 2H), 4.17-4.04 (m, 2H), |
| | | 3.48 (s, 3H), 2.78-2.74 (m, 2H), 2.03-1.88 (m, 3H), 0.90-0.80 (m, 2H), 0.79- |
| | | 0.72 (m, 2H) ppm |
| 722 | 572.2 | 1H NMR (400 MHz, DMSO-d6) δ = 9.60-9.57(m, 1H), 9.11 (s, 1H), 8.53 (d, J = 1.6 |
| | | Hz, 1H), 8.46 (d, J = 9.2 Hz, 1H), 8.32 (d, J = 9.2 Hz, 1H), 8.25 (d, J = 7.6 Hz, 1H), |
| | | 7.73 (d, J = 7.6 Hz, 1H), 7.50 (s, 1H), 7.24 (d, J = 8.0 Hz, 1H), 6.89 (d, J = 8.0 Hz, |
| | | 1H), 4.98 (s, 2H), 4.74 (d, J = 5.6 Hz, 2H), 4.33 (s, 4H), 4.26-4.20 (m, 2H), 3.71- |
| | | 3.65 (m, 2H), 3.57-3.51 (m, 1H), 2.29-2.16 (m, 4H), 2.00-1.90 (m, 1H), 1.87- |
| | | 1.77 (m, 1H) ppm |
| 723 | 556.2 | 1H NMR (400 MHz, DMSO-d6) δ = 9.60-9.57 (m, 1H), 9.08 (s, 1H), 8.54 (d, J = |
| | | 2.0 Hz, 1H), 8.34 (s, 1H), 8.26-8.25 (m, 1H), 8.18 (d, J = 9.2 Hz, 1H), 8.12-811 |
| | | (m, 1H), 8.04-8.03 (m, 1H), 7.92 (d, J = 9.2 Hz, 1H), 7.74 (d, J = 7.6 Hz, 1H), 7.50 |
| | | (s, 1H), 7.14-7.13 (m, 1H), 4.99 (s, 2H), 4.74 (d, J = 5.6 Hz, 2H), 4.27-4.20 (m, |
| | | 2H), 4.14-4.08 (m, 2H), 3.73-3.67 (m, 2H), 2.38-2.32 (m, 2H), 2.14-1.98 (m, 6H) ppm |
| 724 | 565.3 | 1H NMR (400 MHz, DMSO-d6) δ = 9.80-9.77 (m, 1H), 9.48 (d, J = 2.0 Hz, 1H), |
| | | 9.04 (s, 1H), 8.89 (s, 1H), 8.14 (d, J = 9.2 Hz, 1H), 7.99 (d, J = 2.4 Hz, 1H), 7.91 (d, |
| | | J = 9.2 Hz, 1H), 7.78-7.47 (m, 2H), 7.27 (d, J = 2.4 Hz, 1H), 4.77 (d, J = 5.6 Hz, |
| | | 2H), 4.18-4.03 (m, 2H), 3.48 (s, 3H), 2.79-2.76 (m, 2H), 2.00-1.86 (m, 3H), 1.01- |
| | | 0.91 (m, 2H), 0.75-0.66 (m, 2H) ppm |
| 725 | 556.2 | 1H NMR (400 MHz, DMSO-d6) δ = 9.60-9.51 (m, 1H), 8.99 (s, 1H), 8.53-8.46 (m, |
| | | 2H), 8.43 (br s, 1H), 8.27-8.15 (m, 2H), 7.72 (d, J = 7.8 Hz, 1H), 7.42-7.34 (m, |
| | | 2H), 7.15 (s, 1H), 4.97 (s, 2H), 4.69 (d, J = 5.6 Hz, 2H), 4.27-4.18 (m, 2H), 3.99 (t, |
| | | J = 6.2 Hz, 2H), 3.71-3.65 (m, 2H), 2.73 (1, J = 6.3 Hz, 2H), 2.54 (s, 1H), 2.10- |
| | | 2.00 (m, 1H), 1.90 (quin, J = 6.2 Hz, 2H), 0.99-0.79 (m, 4H) ppm |
| 726 | 619.2 | 1H NMR (400 MHz, DMSO-d6) δ = 9.55-9.54 (m, 1H), 8.99 (s, 1H), 8.51 (d, J = |
| | | 1.6 Hz, 1H), 8.24-8.22 (m, 1H), 8.16 (d, J = 9.0 Hz, 1H), 7.73 (d, J = 7.8 Hz, 1H), |
| | | 7.48-7.35 (m, 2H), 7.23-7.07 (m, 2H), 4.97 (s, 2H), 4.70 (br d, J = 5.6 |
| | | Hz, 2H), 4.66-4.64 (m, 2H), 4.38 (t, J = 6.0 Hz, 2H), 4.22-4.20 (m, 2H), 4.18 (d, |
| | | J = 6.8 Hz, 2H), 3.98-3.96 (m, 2H), 3.73-3.65 (m, 3H), 2.66-2.64 (m, 2H), 2.00- |
| | | 1.80 (m, 2H) ppm |
| 727 | 520.1 | 1H NMR (400 MHz, DMSO-d6) δ = 9.58-9.55 (m, 1H), 9.09 (s, 1H), 9.01 (d, J = |
| | | 9.2 Hz, 1H), 8.52 (d, J = 1.6 Hz, 1H), 8.39 (d, J = 9.2 Hz, 1H), 8.25 (d, J = 1.6 Hz, |
| | | 1H), 8.11 (d, J = 1.2 Hz, 1H), 7.73 (d, J = 7.6 Hz, 1H), 7.70-7.64 (m, 1H), 7.48 (s, |
| | | 1H), 4.98 (s, 2H), 4.73 (d, J = 5.6 Hz, 2H), 4.39-4.37(m, 2H), 4.27-4.18 (m, 2H), |
| | | 3.73-3.64 (m, 2H), 3.20-3.16 (m, 2H) ppm |
| 728 | 587.4 | 1H NMR (400 MHz, DMSO-d6) δ 9.51 (t, J = 5.8 Hz, 1H), 9.35 (s, 1H), 8.64 (d, J = |
| | | 1.5 Hz, 1H), 8.45-8.35 (m, 2H), 8.25-8.16 (m, 2H), 7.88 (s, 1H), 7.73 (dd, J = |
| | | 8.5, 7.5 Hz, 1H), 7.50 (d, J = 7.4 Hz, 1H), 6.94 (d, J = 8.5 Hz, 1H), 5.09 (s, 2H), |
| | | 4.80 (d, J = 5.8 Hz, 2H), 4.34 (d, J = 12.2 Hz, 2H), 4.28-4.21 (m, 2H), 3.75-3.64 |
| | | (m, 1H), 3.68 (s, 3H), 1.24 (d, J = 6.2 Hz, 6H). |
| 729 | 557.4 | 1H NMR (400 MHz, Methanol-d4) δ 9.08 (s, 1H), 8.60 (d, J = 1.9 Hz, 1H), 8.51 (s, |
| | | 1H), 8.19 (dd, J = 7.8, 1.9 Hz, 1H), 7.99-7.89 (m, 2H), 7.67 (s, 1H), 7.66-7.59 |
| | | (m, 2H), 7.03 (d, J = 8.0 Hz, 1H), 6.71 (d, J = 8.0 Hz, 1H), 5.04 (s, 2H), 4.84 (s, 2H), |
| | | 4.37-4.29 (m, 4H), 4.03-3.97 (m, 2H), 3.52 (s, 1H), 3.55-3.48 (m, 1H), 1.88- |
| | | 1.80 (m, 1H), 0.73 (tt, J = 7.6, 2.5 Hz, 4H). |
| 730 | 570.2 | 1H NMR (400 MHz, DMSO-d6) δ = 9.58-9.57 (m, 1H), 9.07 (s, 1H), 8.54 (s, 1H), |
| | | 8.33 (s, 1H), 8.26-8.24 (m, 1H), 8.20 (d, J = 9.2 Hz, 1H), 8.06 (d, J = 9.2 Hz, 1H), |
| | | 7.74 (d, J = 7.6 Hz, 1H), 7.50-7.48 (m, 2H), 6.87 (d, J = 7.6 Hz, |
| | | 1H), 4.99 (s, 2H), 4.74 (brd, J = 5.6 Hz, 2H), 4.27-4.20 (m, 2H), 4.19-4.08 (m, |
| | | 2H), 3.71-3.66 (m, 2H), 3.53 (s, 1H), 2.79-2.78 (m, 2H), 2.26-2.16 (m, 4H), 2.02- |
| | | 1.91 (m, 3H), 1.83-1.75 (m, 1H) ppm |
| 731 | 563.3 | 1H NMR (400 MHz, DMSO-d6) δ = 9.56-9.55 (m, 1H), 8.98 (s, 1H), 8.51 (d, J = |
| | | 1.8 Hz, 1H), 8.43 (s, 1H), 8.24-8.23 (m, 1H), 8.14 (d, J = 9.2 Hz, 1H), 7.73 (d, J = |
| | | 7.8 Hz, 1H), 7.44-7.29 (m, 2H), 7.11 (d, J = 7.8 Hz, 2H), 4.97 (s, 2H), |
| | | 4.69 (d, J = 6.0 Hz, 2H), 4.26-4.19 (m, 2H), 4.00-3.98 (m, 2H), 3.87 (d, J = 1.6 |
| | | Hz, 3H), 3.70-3.66 (m, 2H), 2.73-2.70 (m, 2H), 1.88-1.84 (m, 2H) ppm |
| 732 | 560.2 | 1H NMR (400 MHz, DMSO-d6) δ = 9.66-9.54 (m, 1H), 9.03 (s, 1H), 8.53 (d, J = |
| | | 1.6 Hz, 1H), 8.36 (s, 1H), 8.29-8.21 (m, 1H), 8.13 (d, J = 9.2 Hz, 1H), 7.97 (d, J = |
| | | 9.2 Hz, 1H), 7.73 (d, J = 7.6 Hz, 1H), 7.45 (s, 1H), 6.70 (s, 1H), 4.98 (s, 2H), 4.72 |
| | | (d, J = 6.0 Hz, 2H), 4.28-4.19 (m, 2H), 4.16-4.07 (m, 2H), 3.86 (s, 3H), 3.72- |
| | | 3.65 (m, 2H), 2.65-2.59 (m, 2H), 2.34 (s, 3H), 1.98-1.83 (m, 2H) ppm |
| 733 | 560.2 | 1H NMR (400 MHz, DMSO-d6) δ = 9.72-9.71 (m, 1H), 9.44-9.13 (m, 1H), 8.98 (s, |
| | | 1H), 8.79 (d, J = 2.0 Hz, 1H), 8.33-8.25 (m, 1H), 8.14 (d, J = 9.2 Hz, 1H), 7.48- |
| | | 7.42 (m, 1H), 7.39 (d, J = 9.2 Hz, 1H), 7.19-7.13 (m, 1H), 6.93 (d, J = 8.0 Hz, 1H), |
| | | 6.77 (d, J = 8.0 Hz, 1H), 5.10-4.99 (m, 2H), 4.77-4.70 (m, 2H), 4.39-4.32 (m, |
| | | 1H), 4.12-4.03 (m, 3H), 3.87-3.79 (m, 4H), 2.66 (d, J = 6.0 Hz, 2H), 1.97-1.78 |
| | | (m, 2H), 1.23-1.19 (m, 3H) ppm |
| 734 | 556.1 | 1H NMR (400 MHz, DMSO-d6) δ = 9.61-9.48 (m, 1H), 9.04 (s, 1H), 8.52 (d, J = |
| | | 1.6 Hz, 1H), 8.32 (br s, 1H), 8.25-8.24 (m, 1H), 8.15 (d, J = 9.2 Hz, 1H), 7.95- |
| | | 7.85 (m, 2H), 7.73 (d, J = 7.8 Hz, 1H), 7.46 (s, 1H), 7.07 (d, J = 2.2 Hz, 1H), 4.97 |
| | | (s, 2H), 4.72 (d, J = 5.6 Hz, 2H), 4.28-4.20 (m, 4H), 3.69-3.66 (m, 2H), 2.22 (s, |
| | | 3H), 1.87-1.79 (m, 2H), 1.10-1.03 (m, 2H), 0.96-0.89 (m, 2H) ppm |
| 735 | 601.2 | 1H NMR (400 MHz, DMSO-d6) δ = 9.55-9.52 (m, 1H), 8.99 (s, 1H), 8.52 (d, J = |
| | | 2.0 Hz, 1H), 8.25-8.23 (m, 1H), 8.15 (d, J = 9.6 Hz, 1H), 7.73 (d, J = 8.0 Hz, 1H), |
| | | 7.46 (d, J = 9.2 Hz, 1H), 7.40 (s, 1H), 7.15 (d, J = 8.4 Hz, 1H), 6.96 (d, J = 2.4 Hz, |
| | | 1H), 6.73-6.70 (m, 1H), 5.03-4.91 (m, 3H), 4.70 (d, J = 5.6 Hz, 2H), 4.57-4.40 |
| | | (m, 2H), 4.29-4.20 (m, 2H), 4.13-4.06 (m, 1H), 4.05-3.96 (m, 3H), 3.71-3.65 |
| | | (m, 2H), 2.71-2.60 (m, 4H), 1.93-1.86 (m, 2H) ppm |
| 736 | 549.1 | 1H NMR (400 MHz, DMSO-d6) δ = 9.56-9.53 (m, 1H), 9.01 (s, 1H), 8.52 (d, J = |
| | | 1.6 Hz, 1H), 8.39 (s, 1H), 8.25-8.23 (m, 1H), 8.17 (d, J = 9.2 Hz, 1H), 7.73 (d, J = |
| | | 7.6 Hz, 1H), 7.43-7.37 (m, 2H), 7.33-7.27 (m, 1H), 7.26-7.19 (m, 1H), 6.80 (br d, |
| | | J = 8.8 Hz, 1H), 4.98 (s, 2H), 4.71 (br d, J = 5.6 Hz, 2H), 4.26-4.21 (m, 2H), 3.70- |
| | | 3.65 (m, 3H), 3.52-3.45 (m, 2H), 2.73 (br d, J = 4.0 Hz, 2H), 1.97-1.80 (m, 2H) ppm |
| 737 | 536.1 | 1H NMR (400 MHz, DMSO-d6) δ = 9.59-9.58 (m, 1H), 9.10 (s, 1H), 8.52 (d, J = |
| | | 1.6 Hz, 1H), 8.30 (d, J = 9.2 Hz, 1H), 8.25-8.24 (m, 1H), 8.19 (d, J = 9.2 Hz, 1H), |
| | | 7.95 (d, J = 2.8 Hz, 1H), 7.73 (d, J = 7.8 Hz, 1H), 7.50 (s, 1H), 7.45-7.43 (m, 1H), |
| | | 4.97 (s, 2H), 4.73 (br d, J = 5.6 Hz, 2H), 4.36-4.34 (m, 4H), 4.28-4.18 (m, 2H), |
| | | 3.71-3.65 (m, 2H) ppm |
| 738 | 550.2 | 1H NMR (400 MHz, DMSO-d6) δ = 9.59-9.58 (m, 1H), 9.10 (s, 1H), 8.51 (d, J = |
| | | 1.6 Hz, 1H), 8.30 (d, J = 9.2 Hz, 1H), |
| | | 8.26-8.24 (m, 1H), 8.19 (d, J = 9.2 Hz, 1H), 7.95 (d, J = 2.6 Hz, 1H), 7.74 (d, J = |
| | | 7.8 Hz, 1H), 7.51 (s, 1H), 7.45-7.43 (m, 1H), 4.96 (s, 2H), 4.73 (br d, J = 5.6 Hz, |
| | | 2H), 4.36 (br d, J = 3.4 Hz, 4H), 4.29-4.28 (m, 1H), 4.01-3.98 (m, 1H), 3.65-3.64 |
| | | (m, 1H), 1.16 (d, J = 7.2 Hz, 3H) ppm |
| 739 | 558.1 | 1H NMR (400 MHz, DMSO-d6) δ = 9.53-9.52 (m, 1H), 9.03 (s, 1H), 8.57-8.47 (m, |
| | | 2H), 8.32-8.18 (m, 2H), 7.71 (d, J = 7.8 Hz, 1H), 7.50 (d, J = 9.2 Hz, 1H), 7.39 (s, |
| | | 1H), 6.88 (s, 1H), 4.96 (s, 2H), 4.69 (d, J = 5.8 Hz, 2H), 4.37-4.30 (m, 2H), 4.25- |
| | | 4.17 (m, 4H), 3.69-3.62 (m, 2H), 2.03-1.97 (m, 1H), 0.90-0.85 (m, 4H) ppm |
| 740 | 600.3 | 1H NMR (400 MHz, DMSO-d6) δ = 9.62-9.60 (m, 1H), 9.40 (s, 1H), 8.68-8.61 (m, |
| | | 2H), 8.41 (d, J = 1.6 Hz, 1H), 8.27-8.24 (m, 1H), 7.92 (d, J = 7.2 Hz, 1H), 7.83 (s, |
| | | 1H), 7.77-7.72 (m, 2H), 7.03 (d, J = 8.4 Hz, 1H), 5.08 (s, 2H), 4.81 (br d, J = 5.6 |
| | | Hz, 2H), 4.35-4.02 (m, 4H), 3.69-3.64 (m, 2H), 2.47 (br s, 2H), 1.33-1.11 (m, 9H) ppm |
| 741 | 550.2 | 1H NMR (400 MHz, DMSO-d6) δ = 9.59-9.58 (m, 1H), 9.08 (s, 1H), 8.51 (d, J = |
| | | 1.8 Hz, 1H), 8.36 (s, 1H), 8.29-8.24 (m, 3H), 7.82-7.69 (m, 2H), 7.49 (s, 1H), 7.20 |
| | | (d, J = 1.8 Hz, 1H), 4.73 (br d, J = 5.7 Hz, 2H), 4.32 (s, 4H), 3.64-3.60 (m, 1H), |
| | | 2.25 (s, 3H), 1.15 (d, J = 7.2 Hz, 3H) ppm |
| 742 | 581.1 | 1H NMR (400 MHz, DMSO-d6) δ = 9.58-9.57 (m, 1H), 9.04 (s, 1H), 8.52 (d, J = |
| | | 1.6 Hz, 1H), 8.30 (s, 1H), 8.25-8.24 (m, 1H), 8.18 (d, J = 9.2 Hz, 1H), 7.92 (d, J = |
| | | 9.2 Hz, 1H), 7.73 (d, J = 7.8 Hz, 1H), 7.62-7.60 (m, 1H), 7.46 (s, 1H), 7.27 (d, J = |
| | | 8.2 Hz, 1H), 7.01-6.98 (m, 1H), 6.48-6.11 (m, 1H), 4.97 (s, 2H), 4.72 (d, J = 5.6 |
| | | Hz, 2H), 4.23-4.20 (m, 4H), 3.83-3.80 (m, 2H), 3.71-3.65 (m, 2H), 3.56-3.52 |
| | | (m, 2H) ppm |
| 743 | 516.1 | 1H NMR (400 MHz, DMSO-d6) δ = 9.58-9.57 (m, 1H), 9.15 (d, J = 9.2 Hz, 1H), |
| | | 9.08 (s, 1H), 8.53 (d, J = 1.6 Hz, 1H), 8.36 (d, J = 9.2 Hz, 1H), 8.25-8.15 (m, 1H), |
| | | 8.03 (d, J = 5.2 Hz, 1H), 7.74 (d, J = 7.8 Hz, 1H), 7.47 (s, 1H), 6.81 (d, |
| | | J = 5.2 Hz, 1H), 4.98 (s, 2H), 4.73 (d, J = 5.8 Hz, 2H), 4.34-4.30 (m, 2H), 4.27- |
| | | 4.19 (m, 2H), 3.73-3.64 (m, 2H), 3.08-3.06 (m, 2H), 2.24 (s, 3H) ppm |
| 744 | 545.2 | 1H NMR (400 MHz, DMSO-d6) δ = 9.63-9.40 (m, 1H), 8.97 (s, 1H), 8.51 (s, 1H), |
| | | 8.49 (s, 1H), 8.29-8.20 (m, 2H), 7.72 (d, J = 8.0 Hz, 1H), 7.48 (d, J = 8.4 Hz, 1H), |
| | | 7.36 (s, 1H), 7.15 (s, 1H), 4.98 (d, J = 6.8 Hz, 4H), 4.69 (d, J = 5.6 Hz, 2H), 4.28- |
| | | 4.14 (m, 6H), 3.67 (d, J = 4.0 Hz, 2H), 1.74-1.59 (m, 1H), 0.83-0.77 (m, 2H), 0.51- |
| | | 0.48 (m, 2H) ppm |
| 745 | 619.1 | 1H NMR (400 MHz, DMSO-d6) δ = 9.62-9.53 (m, 1H), 9.00 (s, 1H), 8.52 (s, 1H), |
| | | 8.25 (d, J = 7.6 Hz, 1H), 8.16 (d, J = 9.2 Hz, 1H), 7.73 (d, J = 8.0 Hz, 1H), 7.47 (d, J |
| | | = 9.2 Hz, 1H), 7.40 (s, 1H), 7.17 (d, J = 8.4 Hz, 1H), 6.96 (s, 1H), 6.73 (d, J = 8.4 |
| | | Hz, 1H), 4.97 (s, 2H), 4.73-4.59 (m, 6H), 4.42-4.30 (m, 2H), 4.22 (s, 2H), 3.99- |
| | | 3.96 (m, 2H), 3.68 (s, 2H), 2.68-2.65 (m, 2H), 1.91-1.87 (m, 2H) ppm |
| 746 | 572.2 | 1H NMR (400 MHz, DMSO-d6) δ = 9.60-9.57 (m, 1H), 9.07 (s, 1H), 8.53 (s, 1H), |
| | | 8.47 (br s, 1H), 8.29-8.17 (m, 3H), 7.74 (d, J = 8.0 Hz, 1H), 7.56-7.46 (m, 2H), |
| | | 6.48 (d, J = 8.0 Hz, 1H), 4.98 (s, 2H), 4.74 (br d, J = 5.2 Hz, 2H), 4.23 (br d, J = 5.2 |
| | | Hz, 2H), 4.14 (br t, J = 5.6 Hz, 2H), 4.00 (br d, J = 2.8 Hz, 1H), 3.68 (br d, J = 4.4 |
| | | Hz, 2H), 2.76 (br t, J = 6.4 Hz, 2H), 2.01-1.92 (m, 2H), 0.74-0.62 (m, 4H) ppm |
| 747 | 615.3 | 1H NMR (400 MHz, DMSO-d6) δ = 9.55-9.54 (m, 1H), 8.99 (s, 1H), 8.52 (d, J = |
| | | 1.8 Hz, 1H), 8.34-8.29 (m, 1H), 8.24-8.22 (m, 1H), 8.15 (d, J = 9.2 Hz, 1H), 7.73 |
| | | (d, J = 7.8 Hz, 1H), 7.45 (d, J = 9.2 Hz, 1H), 7.39 (s, 1H), 7.13 (d, J = |
| | | 8.4 Hz, 1H), 6.90 (d, J = 2.4 Hz, 1H), 6.68-6.65 (m, 1H), 4.97 (s, 2H), 4.70 (d, J = |
| | | 5.8 Hz, 2H), 4.22-4.20 (m, 2H), 4.10-4.08 (m, 1H), 3.97-3.95 (m, 2H), 3.92- |
| | | 3.81 (m, 2H), 3.78-3.71 (m, 1H), 3.70-3.61 (m, 3H), 2.69-2.63 (m, 2H), 1.99- |
| | | 1.73 (m, 5H), 1.64 (s, 1H) ppm |
| 748 | 571.3 | 1H NMR (400 MHz, DMSO-d6) δ = 9.57-9.54 (m, 1H), 9.01 (s, 1H), 8.52 (d, J = |
| | | 1.2 Hz, 1H), 8.25 (d, J = 1.6 Hz, 1H), 8.15 (d, J = 9.2 Hz, 1H), 7.97 (d, J = 9.2 Hz, |
| | | 1H), 7.73 (d, J = 7.6 Hz, 1H), 7.50 (s, 1H), 7.43 (s, 1H), 7.28 (s, 1H), 4.97 (s, 2H), |
| | | 4.71 (d, J = 5.6 Hz, 2H), 4.24-4.21 (m, 4H), 3.78-3.60 (m, 2H), 3.39-3.35 (m, |
| | | 2H), 2.46-2.41 (m, 1H), 2.25 (s, 3H), 0.93-0.79 (m, 2H), 0.66-0.55 (m, 2H) ppm |
| 749 | 546.2 | 1H NMR (400 MHz, DMSO-d6) δ = 9.82-9.67 (m, 1H), 9.28 (d, J = 2.4 Hz, 1H), |
| | | 9.02 (s, 1H), 8.78 (d, J = 2.4 Hz, 1H), 8.43 (s, 1H), 8.24 (d, J = 9.2 Hz, 1H), 7.52 (d, |
| | | J = 9.2 Hz, 1H), 7.46 (s, 1H), 7.35 (d, J = 8.2 Hz, 1H), 6.80-6.71 (m, 2H), 5.10- |
| | | 4.99 (m, 2H), 4.72 (br d, J = 6.0 Hz, 2H), 4.35-4.33 (m, 1H), 4.30-4.17 (m, 4H), |
| | | 4.12-4.03 (m, 1H), 3.83-3.82 (m, 1H), 2.26 (s, 3H), 1.21 (d, J = 7.0 Hz, 3H) ppm |
| 750 | 563.3 | 1H NMR (400 MHz, DMSO-d6) δ = 9.54-9.53 (m, 1H), 9.02 (s, 1H), 8.51 (d, J = 1.6 |
| | | Hz, 1H), 8.28-8.17 (m, 2H), 7.72 (d, J = 7.8 Hz, 1H), 7.45 (d, J = 9.2 Hz, 1H), 7.41 |
| | | (s, 1H), 6.77-6.75 (m, 1H), 6.67-6.65 (m, 1H), 4.97 (s, 2H), 4.71 (d, J = 5.6 Hz, |
| | | 2H), 4.26-4.19 (m, 2H), 4.06-3.99 (m, 2H), 3.82 (s, 3H), 3.72-3.62 (m, 2H), 2.60- |
| | | 2.58 (m, 2H), 1.92-1.81 (m, 2H) ppm |
| 751 | 576.1 | 1H NMR (400 MHz, DMSO-d6) δ = 9.59-9.56 (m, 1H), 9.11 (s, 1H), 8.53 (d, J = |
| | | 1.6 Hz, 1H), 8.32 (d, J = 9.2 Hz, 1H), 8.26-8.24 (m, 1H), 8.07 (d, J = 9.2 Hz, 1H), |
| | | 7.74 (d, J = 7.6 Hz, 1H), 7.49 (s, 1H), 7.35 (d, J = 10.0 Hz, 1H), 4.98 (s, 2H), 4.74 |
| | | (d, J = 5.6 Hz, 2H), 4.32-4.28 (m, 4H), 4.26-4.21 (m, 2H), 3.71-3.65 (m, 2H), |
| | | 2.23-2.15 (m, 1H), 0.99-0.91 (m, 2H), 0.90-0.84 (m, 2H) ppm |
| 752 | 544.3 | 1H NMR (400 MHz, DMSO-d6) δ = 9.59-9.56 (m, 1H), 9.08 (s, 1H), 8.54 (d, J = |
| | | 1.6 Hz, 1H), 8.26-8.25 (m, 1H), 8.18 (d, J = 9.2 Hz, 1H), 8.12-8.11 (m, 1H), 7.91 |
| | | (d, J = 9.2 Hz, 1H), 7.82-7.81 (m, 1H), 7.74 (d, J = 8.0 Hz, 1H), 7.50 (s, 1H), 7.09- |
| | | 7.08 (m, 1H), 4.99 (s, 2H), 4.74 (d, J = 5.6 Hz, 2H), 4.26-4.19 (m, 2H), 4.19-4.12 |
| | | (m, 2H), 3.71-3.66 (m, 2H), 1.87-1.79 (m, 2H), 1.31 (s, 6H) ppm |
| 753 | 572.3 | 1H NMR (400 MHz, DMSO-d6) δ = 9.579.56 (m, 1H), 9.08 (s, 1H), 8.53 (d, J = 1.6 |
| | | Hz, 1H), 8.32-8.22 (m, 3H), 7.80 (d, J = 1.8 Hz, 1H), 7.73 (d, J = 7.8 Hz, 1H), 7.48 |
| | | (s, 1H), 7.27 (d, J = 1.8 Hz, 1H), 4.98 (s, 2H), 4.73 (d, J = 5.8 Hz, 2H), 4.33 (s, 4H), |
| | | 4.27-4.18 (m, 2H), 3.71-3.65 (m, 2H), 3.50-3.30 (m, 1H), 2.28-2.10 (m, 2H), |
| | | 2.17-2.06 (m, 2H), 2.03-1.92 (m, 1H), 1.87-1.80 (m, 1H) ppm |
| 754 | 587.3 | 1H NMR (400 MHz, DMSO-d6) δ = 9.56-9.54 (m, 1H), 9.00 (s, 1H), 8.52 (d, J = |
| | | 2.0 Hz, 1H), 8.26-8.23 (m, 1H), 8.18 (s, 2H), 8.15 (s, 1H), 7.73 (d, J = 7.6 Hz, 1H), |
| | | 7.53-7.25 (m, 2H), 6.56 (d, J = 2.4 Hz, 1H), 4.98 (s, 2H), 4.71 (d, J = 5.6 Hz, 2H), |
| | | 4.36-4.18 (m, 6H), 3.73-3.65 (m, 2H), 3.24-3.21 (m, 4H), 2.03-1.86 (m, 4H) ppm |
| 755 | 558.3 | 1H NMR (400 MHz, DMSO-d6) δ = 9.59-9.56 (m, 1H), 9.16 (s, 1H), 8.51 (d, J = |
| | | 1.6 Hz, 1H), 8.42 (d, J = 8.8 Hz, 1H), 8.25-8.22 (m, 1H), 8.19 (s, 1H), 8.01 (s, 1H), |
| | | 7.73 (d, J = 7.6 Hz, 1H), 7.67 (d, J = 9.2 Hz, 1H), 7.50 (d, J = 7.6 Hz, 2H), 4.97 (s, |
| | | 2H), 4.74 (d, J = 5.6 Hz, 2H), 4.32-4.30 (m, 2H), 4.26-4.20 (m, 2H), 4.18-4.11 |
| | | (m, 2H), 3.69-3.65 (m, 2H), 2.00-1.89 (m, 1H), 0.85-0.75 (m, 4H) ppm |
| 756 | 597.3 | 1H NMR (400 MHz, DMSO-d6) δ = 9.57-9.55 (m, 1H), 9.01 (s, 1H), 8.52 (d, J = |
| | | 1.6 Hz, 1H), 8.33 (s, 1H), 8.25 (d, J = 7.6 Hz, 1H), 8.15 (d, J = 9.2 Hz, 1H), 7.96 (d, |
| | | J = 9.2 Hz, 1H), 7.73 (d, J = 7.6 Hz, 1H), 7.49 (d, J = 2.0 Hz, 1H), 7.43 (s, 1H), 7.10 |
| | | (d, J = 2.0 Hz, 1H), 4.98 (s, 2H), 4.71 (d, J = 5.6 Hz, 2H), 4.24-4.21 (m, 4H), 3.72- |
| | | 3.65 (m, 2H), 3.37 (d, J = 5.6 Hz, 2H), 2.47-2.45 (m, 1H), 1.96-1.86 (m, 1H), 1.00- |
| | | 0.92 (m, 2H), 0.89-0.81 (m, 2H), 0.74-0.66 (m, 2H), 0.63-0.54 (m, 2H) ppm |
| 757 | 593.2 | 1H NMR (400 MHz, DMSO-d6) δ = 9.55-9.54 (m, 1H), 9.12-9.03 (m, 1H), 8.50 |
| | | (d, J = 1.8 Hz, 1H), 8.40 (br s, 1H), 8.32 (d, J = 9.2 Hz, 1H), 8.23 (d, J = 7.8 Hz, |
| | | 1H), 7.75-7.69 (m, 1H), 7.38 (s, 1H), 7.13-7.12 (m, 1H), 6.72-6.70 (m, 1H), 4.97 |
| | | (s, 2H), 4.71 (d, J = 5.8 Hz, 2H), 4.28-4.15 (m, 6H), 3.69-3.64 (m, 2H), 1.77- |
| | | 1.68 (m, 1H), 0.92-0.85 (m, 2H), 0.75-0.65 (m, 2H) ppm |
| 758 | 623.2 | 1H NMR (400 MHz, DMSO-d6) δ = 9.57-9.54 (m, 1H), 9.02 (s, 1H), 8.52 (d, J = |
| | | 1.6 Hz, 1H), 8.40-8.35 (m, 1H), 8.26-8.24 (m, 1H), 8.23-8.17 (m, 2H), 7.73 (d, |
| | | J = 7.6 Hz, 1H), 7.50-7.40 (m, 2H), 6.73 (d, J = 2.8 Hz, 1H), 4.98 (s, 2H), 4.71 (d, |
| | | J = 5.6 Hz, 2H), 4.32-4.29 (m, 4H), 4.26-4.18 (m, 2H), 3.75-3.66 (m, 4H), 3.51- |
| | | 3.47 (m, 2H), 2.56-2.53 (m, 2H) ppm |
| 759 | 615.4 | 1H NMR (400 MHz, DMSO-d6) δ = 9.56-9.55(m, 1H), 8.99 (s, 1H), 8.52 (d, J = 2.0 |
| | | Hz, 1H), 8.26-8.23 (m, 1H), 8.15 (d, J = 8.8 Hz, 1H), 7.73 (d, J = 8.0 Hz, 1H), 7.45 |
| | | (d, J = 9.2 Hz, 1H), 7.39 (s, 1H), 7.13 (d, J = 8.3 Hz, 1H), 6.91 (d, J = 2.4 Hz, 1H), |
| | | 6.69 (d, J = 8.4 Hz, 1H), 4.97 (s, 2H), 4.70 (d, J = 5.6 Hz, 2H), 4.27-4.18 (m, 2H), |
| | | 3.98-3.97 (m, 2H), 3.90-3.84 (m, 1H), 3.83-3.77 (m, 1H), 3.77-3.71 (m, 2H), |
| | | 3.70-3.66 (m, 2H), 3.65-3.58 (m, 1H), 3.49 (d, J = 8.8 Hz, 1H), 2.70-2.63 (m, |
| | | 2H), 2.62-2.56 (m, 1H), 2.02-1.93 (m, 1H), 1.93-1.85 (m, 2H), 1.66-1.55 (m, 1H) ppm |
| 760 | 575.2 | 1H NMR (400 MHz, DMSO-d6) δ = 9.61-9.49 (m, 1H), 9.10-9.00 (m, 1H), 8.52- |
| | | 8.48 (m, 1H), 8.34-8.26 (m, 1H), 8.26-8.20 (m, 1H), 7.71 (d, J = 7.6 Hz, 1H), 7.46- |
| | | 7.37 (m, 1H), 7.13-7.03 (m, 1H), 6.80-6.55 (m, 2H), 4.96 (s, 2H), 4.71 (d, J = |
| | | 5.6 Hz, 2H), 4.22 (s, 6H), 3.71-3.63 (m, 2H), 2.00-1.85 (m, 1H), 0.98-0.85 (m, |
| | | 2H), 0.73-0.61 (m, 2H) ppm |
| 761 | 546.3 | 1H NMR (400 MHz, DMSO-d6) δ = 9.56-9.53 (m, 1H), 9.03 (s, 1H), 8.51 (d, J = |
| | | 1.6 Hz, 1H), 8.31 (s, 1H), 8.27 (s, 1H), 8.26-8.22 (m, 2H), 8.03 (s, 1H), 7.72 (d, J = |
| | | 7.6 Hz, 1H), 7.44 (d, J = 9.2 Hz, 1H), 7.38 (s, 1H), 4.97 (s, 2H), 4.70 (d, J = 6.0 Hz, |
| | | 2H), 4.28-4.19 (m, 2H), 4.09-4.00 (m, 2H), 3.92 (s, 3H), 3.70-3.65 (m, 2H), 2.69- |
| | | 2.65 (m, 2H), 1.96-1.84 (m, 2H) ppm |
| 762 | 527.2 | 1H NMR (400 MHz, DMSO-d6) δ = 9.59-9.58 (m, 1H), 9.28 (s, 1H), 8.52 (d, J = 1.8 |
| | | Hz, 1H), 8.35 (d, J = 8.6 Hz, 1H), 8.25 (d, J = 7.8 Hz, 1H), 8.19-8.09 (m, 1H), 7.72 |
| | | (d, J = 7.8 Hz, 1H), 7.68 (s, 1H), 7.53 (d, J = 8.4 Hz, 2H), |
| | | 7.14-6.90 (m, 1H), 4.97 (s, 2H), 4.78 (d, J = 5.8 Hz, 2H), 4.224.10(m, 2H), 3.71- |
| | | 3.64 (m, 2H), 2.85-2.75 (m, 1H), 2.72-2.61 (m, 1H), 2.27 (d, J = 8.6 Hz, 1H), 2.14- |
| | | 2.05 (m, 1H), 2.01-1.90 (m, 1H), 1.84-1.77 (m, 1H), 1.48-1.40 (m, 1H) ppm |
| 763 | 561.4 | 1H NMR (400 MHz, DMSO-d6) δ = 9.58-9.57 (m, 1H), 9.01 (s, 1H), 8.52 (d, J = |
| | | 1.6 Hz, 1H), 8.25 (d, J = 7.6 Hz, 1H), 8.20 (s, 2H), 7.73 (d, J = 7.6 Hz, 1H), 7.55 (d, |
| | | J = 2.8 Hz, 1H), 7.42 (s, 1H), 6.77 (d, J = 2.8 Hz, 1H), 4.97 (s, 2H), 4.71 (d, J = 5.6 |
| | | Hz, 2H), 4.32 (d, J = 4.8 Hz, 2H), 4.30-4.25 (m, 2H), 4.25-4.20 (m, 2H), 3.72- |
| | | 3.65 (m, 2H), 2.89 (s, 6H) ppm |
| 764 | 549.2 | 1H NMR (400 MHz, DMSO-d6) δ = 9.65-9.47 (m, 1H), 9.06 (s, 1H), 8.50 (d, J = |
| | | 1.6 Hz, 1H), 8.30 (d, J = 9.0 Hz, 1H), 8.26-8.20 (m, 1H), 7.72 (d, J = 7.8 Hz, 1H), |
| | | 7.41 (s, 1H), 7.09-6.90 (m, 1H), 6.76-6.70 (m, 1H), 6.69-6.63 (m, 1H), |
| | | 4.97 (s, 2H), 4.71 (br d, J = 5.4 Hz, 2H), 4.33-4.14 (m, 7H), 3.76-3.61 (m, 2H), |
| | | 2.28 (s, 3H) ppm |
| 765 | 575.2 | 1H NMR (400 MHz, DMSO-d6) δ = 9.56-9.55 (m, 1H), 9.06 (s, 1H), 8.51 (d, J = 1.6 |
| | | Hz, 1H), 8.44-8.39 (m, 1H), 8.29 (d, J = 9.2 Hz, 1H), 8.23 (d, J = 7.8 Hz, 1H), 7.72 |
| | | (d, J = 7.8 Hz, 1H), 7.41 (s, 1H), 7.07-6.90 (m, 1H), 6.81-6.36 (m, 2H), 4.97 (s, |
| | | 2H), 4.71 (br d, J = 5.6 Hz, 2H), 4.23 (br s, 6H), 3.75-3.60 (m, 2H), 1.99-1.82 (m, |
| | | 1H), 1.03-0.86 (m, 2H), 0.75-0.61 (m, 2H) ppm |
| 766 | 584.3 | 1H NMR (400 MHz, DMSO-d6) δ = 9.60-9.59 (m, 1H), 9.09 (s, 1H), 8.53 (d, J = |
| | | 1.6 Hz, 1H), 8.33-8.16 (m, 3H), 7.81 (d, J = 4.8 Hz, 1H), 7.73 (d, J = 7.6 Hz, 1H), |
| | | 7.48 (s, 1H), 6.83 (d, J = 4.8 Hz, 1H), 4.98 (s, 2H), 4.73 (d, J = 5.6 Hz, 2H), 4.36 (d, |
| | | J = 4.8 Hz, 2H), 4.32 (d, J = 4.8 Hz, 2H), 4.26-4.19 (m, 2H), 3.74-3.63 (m, 2H), |
| | | 2.58 (s, 1H), 2.15 (s, 6H) ppm |
| 767 | 552.1 | 1H NMR (400 MHz, METHANOL-d4) δ = 9.10 (s, 1H), 8.61 (d, J = 2.0 Hz, 1H), 8.47- |
| | | 8.43 (m, 1H), 8.35-8.31 (m, 1H), 8.27 (d, J = 9.0 Hz, 1H), 8.218.19 (m, 1H), |
| | | 8.07 (d, J = 9.2 Hz, 2H), 7.71 (s, 1H), 7.64 (d, J = 8.0 Hz, 1H), 7.18-7.17 (m, 1H), |
| | | 5.05 (s, 2H), 4.98-4.95 (m, 2H), 4.44-4.39 (m, 2H), 4.36-4.32 (m, 2H), 3.54- |
| | | 3.51 (m, 2H), 2.64-2.50 (m, 2H) ppm |
| 768 | 546.3 | 1H NMR (400 MHz, DMSO-d6) δ = 9.59-9.58 (m, 1H), 9.13 (s, 1H), 8.51 (d, J = 1.8 |
| | | Hz, 1H), 8.39 (s, 1H), 8.33 (d, J = 9.0 Hz, 1H), 8.24 (d, J = 7.8 Hz, 1H), 7.92 (s, |
| | | 1H), 7.73 (d, J = 7.8 Hz, 1H), 7.63 (d, J = 9.0 Hz, 1H), 7.49 (s, 1H), 6.73 (s, 1H), |
| | | 4.97 (s, 2H), 4.73 (d, J = 5.6 Hz, 2H), 4.28-4.15 (m, 2H), 3.96-3.90 (m, 2H), 3.78 |
| | | (s, 3H), 3.70-3.65 (m, 2H), 2.75-2.67 (m, 2H), 2.01-1.87 (m, 2H) ppm |
| 769 | 588.3 | 1H NMR (400 MHz, DMSO-d6) δ = 9.83-9.69 (m, 1H), 9.35-9.27 (m, 2H), 8.80 |
| | | (d, J = 2.2 Hz, 1H), 8.61 (s, 1H), 8.35 (d, J = 8.6 Hz, 1H), 8.19 (d, J = 8.6 Hz, 1H), |
| | | 7.91 (s, 1H), 7.70-7.60 (m, 1H), 7.45 (d, J = 7.6 Hz, 1H), 6.90 (d, J = 8.4 Hz, 1H), |
| | | 5.14-4.96 (m, 2H), 4.79 (br d, J = 5.4 Hz, 2H), 4.42-4.26 (m, 3H), 4.09-3.99 (m, |
| | | 1H), 3.81 (brd, J = 2.8 Hz, 1H), 3.71-3.62 (m, 2H), 3.38 (br s, 2H), 1.25-1.17 (m, 9H) ppm |
| 770 | 550.3 | 1H NMR (400 MHz, DMSO-d6) δ = 9.59-9.56 (m, 1H), 9.10 (s, 1H), 8.53 (d, J = |
| | | 1.6 Hz, 1H), 8.29 (s, 2H), 8.26-8.25 (m, 1H), 7.74 (d, J = 7.6 Hz, 1H), 7.49 (s, 1H), |
| | | 7.37 (d, J = 9.6 Hz, 1H), 4.98 (s, 2H), 4.74 (d, J = 5.6 Hz, 2H), 4.34 (s, 4H), 4.27- |
| | | 4.21 (m, 2H), 3.71-3.66 (m, 2H), 2.34 (d, J = 2.8 Hz, 3H) ppm |
| 771 | 586.3 | 1H NMR (400 MHz, DMSO-d6) δ = 9.57-9.54 (m, 1H), 9.04 (s, 1H), 8.52 (d, J = |
| | | 2.0 Hz, 1H), 8.25 (d, J = 1.6 Hz, 1H), 8.21 (s, 1H), 8.15 (d, J = 9.2 Hz, 1H), 7.80 (d, |
| | | J = 9.2 Hz, 1H), 7.73 (d, J = 8.0 Hz, 1H), 7.45 (s, 1H), 6.77 (s, 1H), 4.97 (s, 2H), |
| | | 4.72 (d, J = 5.6 Hz, 2H), 4.22-4.21 (m, 2H), 4.12-4.05 (m, 2H), 3.87 (s, 3H), 3.71- |
| | | 3.65 (m, 2H), 2.61-2.58 (m, 2H), 2.04-1.94 (m, 1H), 1.93-1.82 (m, 2H), 0.89- |
| | | 0.75 (m, 4H) ppm |
| 772 | 574.1 | 1H NMR (400 MHz, METHANOL-d4) δ = 9.33 (s, 1H), 8.70-8.58 (m, 3H), 8.48 (d, |
| | | J = 8.4 Hz, 1H), 8.23-8.20 (m, 1H), 7.98 (s, 1H), 7.80 (d, J = 7.6 Hz, 1H), 7.65 (d, |
| | | J = 8.0 Hz, 1H), 5.05 (s, 2H), 4.93 (s, 2H), 4.39-4.32 (m, 2H), 4.30-4.12 (m, 2H), |
| | | 3.77-3.74 (m, 2H), 3.56-3.51 (m, 2H), 3.44 (s, 3H), 3.25-3.24 (m, 2H), 3.15- |
| | | 3.14 (m, 4H) ppm |
| 773 | 587.2 | 1H NMR (400 MHz, DMSO-d6) δ = 9.66-9.57 (m, 1H), 9.10 (s, 1H), 8.43 (d, J = |
| | | 4.4 Hz, 1H), 8.40-8.30 (m, 2H), 8.15-8.13 (m, 1H), 7.44 (s, 1H), 7.20-7.11 (m, |
| | | 1H), 7.10-7.08 (m, 1H), 7.01-6.97 (m, 1H), 5.02 (s, 2H), 4.71 (d, J = 5.2 Hz, 2H), |
| | | 4.33-4.17 (m, 6H), 3.79-3.72 (m, 2H) ppm |
| 774 | 565.2 | 1H NMR (400 MHz, DMSO-d6) δ = 9.56-9.54 (m, 1H), 9.05 (s, 1H), 8.51 (d, J = |
| | | 1.6 Hz, 1H), 8.29 (d, J = 9.2 Hz, 1H), 8.24-8.22 (m, 1H), 7.72 (d, J = 7.6 Hz, 1H), |
| | | 7.40 (s, 1H), 7.08-7.05 (m, 1H), 6.59-6.55 (m, 1H), 6.44-6.43 (m, 1H), 4.97 (s, |
| | | 2H), 4.71 (d, J = 6.0 Hz, 2H), 4.28-4.19 (m, 6H), 3.75 (s, 3H), 3.69-3.65 (m, 2H) ppm |
| 775 | 558.2 | 1H NMR (400 MHz, DMSO-d6) δ = 9.70-9.67 (m, 1H), 8.54-8.43 (m, 2H), 8.23- |
| | | 821 (m, 1H), 7.83-7.69 (m, 2H), 7.63 (s, 1H), 7.38 (d, J = 8.4 Hz, 1H), 6.75-6.59 |
| | | (m, 2H), 5.06-4.88 (m, 4H), 4.34-4.14 (m, 6H), 3.73-3.61 (m, 2H), 1.95-1.80 |
| | | (m, 1H), 0.99-0.87 (m, 2H), 0.71-0.58 (m, 2H) ppm |
| 776 | 547.2 | 1H NMR (400 MHz, DMSO-d6) δ = 9.58-9.55 (m, 1H), 9.06 (s, 1H), 8.52 (d, J = |
| | | 1.6 Hz, 1H), 8.35 (s, 1H), 8.32-8.18 (m, 2H), 7.74 (d, J = 7.6 Hz, 1H), 7.62 (d, J = |
| | | 9.2 Hz, 1H), 7.43 (s, 1H), 7.08 (d, J = 3.2 Hz, 1H), 6.89 (d, J = 8.8 Hz, 1H), 6.68- |
| | | 6.65 (m, 1H), 4.98 (s, 2H), 4.72 (d, J = 5.6 Hz, 2H), 4.24-4.22 (m, 4H), 4.20-4.18 |
| | | (m, 2H), 3.70-3.67 (m, 5H) ppm |
| 777 | 568.3 | 1H NMR (400 MHz, DMSO-d6) δ = 9.58-9.55 (m, 1H), 9.11 (s, 1H), 8.88 (s, 1H), |
| | | 8.50 (d, J = 1.6 Hz, 1H), 8.38 (d, J = 9.2 Hz, 1H), 8.27 (s, 1H), 8.24-8.22 (m, 1H), |
| | | 7.72 (d, J = 7.6 Hz, 1H), 7.61 (d, J = 9.2 Hz, 1H), 7.45 (s, 1H), 7.26 (s, 1H), 7.01- |
| | | 6.72 (m, 1H), 4.97 (s, 2H), 4.72 (d, J = 5.6 Hz, 2H), 4.44-4.42 (m, 2H), 4.27-4.18 |
| | | (m, 4H), 3.70-3.65 (m, 2H) ppm |
| 778 | 566.3 | 1H NMR (400 MHz, DMSO-d6) δ = 9.60-9.57 (m, 1H), 9.12 (s, 1H), 8.53 (d, J = |
| | | 2.0 Hz, 1H), 8.46-8.40 (m, 1H), 8.32 (s, 2H), 8.26-8.24 (m, 1H), 7.73 (d, J = 8.0 |
| | | Hz, 1H), 7.49 (d, J = 9.6 Hz, 2H), 4.98 (s, 2H), 4.74 (br d, J = 6.0 Hz, 2H), 4.33 (s, |
| | | 4H), 4.26-4.19 (m, 2H), 3.71-3.65 (m, 2H), 2.42 (s, 3H) ppm |
| 779 | 590.3 | 1H NMR (400 MHz, DMSO-d6) δ = 9.67-9.55 (m, 1H), 9.40 (s, 1H), 8.72-8.57 (m, |
| | | 2H), 8.50 (d, J = 1.6 Hz, 1H), 8.24 (d, J = 7.6 Hz, 1H), 7.90 (d, J = 7.2 Hz, 1H), 7.80 |
| | | (s, 1H), 7.78-7.70 (m, 2H), 7.03 (d, J = 8.8 Hz, 1H), 5.11-4.89 (m, 1H), 4.81 (d, |
| | | J = 5.6 Hz, 2H), 4.31 (d, J = 11.6 Hz, 2H), 3.72-3.60 (m, 3H), 3.58-3.45 (m, 3H), |
| | | 2.56 (d, J = 4.0 Hz, 2H), 2.47-2.39 (m, 2H), 1.21 (d, J = 6.0 Hz, 6H) ppm |
| 780 | 557.2 | 1H NMR (400 MHz, DMSO-d6) δ = 9.62-9.60 (m, 1H), 9.20 (s, 1H), 8.52 (d, J = |
| | | 1.6 Hz, 1H), 8.35 (d, J = 8.8 Hz, 1H), 8.26-8.24 (m, 1H), 8.19 (s, 1H), 8.07 (d, J = |
| | | 9.2 Hz, 1H), 7.73 (d, J = 7.6 Hz, 1H), 7.56 (s, 1H), 4.97 (s, 2H), 4.76 (d, J = 6.0 Hz, |
| | | 2H), 4.29-4.18 (m, 2H), 4.15-4.05 (m, 2H), 3.71-3.66 (m, 2H), 2.76-2.73 (m, |
| | | 2H), 2.02-1.92 (m, 3H), 0.94-0.80 (m, 4H) ppm |
| 781 | 586.2 | 1H NMR (400 MHz, DMSO-d6) δ = 9.64-9.53 (m, 1H), 9.14 (s, 1H), 8.53 (d, J = |
| | | 1.6 Hz, 1H), 8.41 (br s, 1H), 8.38-8.33 (m, 1H), 8, 31-8.23 (m, 2H), 7.73 (d, J = |
| | | 7.6 Hz, 1H), 7.60 (d, J = 10.4 Hz, 1H), 7.52 (s, 1H), 7.19-6.86 (m, 1H), 5.04-4.93 |
| | | (m, 2H), 4.78-4.71 (m, 2H), 4.45-4.36 (m, 4H), 4.27-4.19 (m, 2H), 3.71-3.65 (m, 2H) ppm |
| 782 | 587.3 | 1H NMR (400 MHz, DMSO-d6) δ = 9.57-9.56 (m, 1H), 9.02 (s, 1H), 8.53 (d, J = 1.8 |
| | | Hz, 1H), 8.28-8.18 (m, 3H), 7.77-7.69 (m, 2H), 7.43 (s, 1H), 6.96 (d, J = 2.4 Hz, |
| | | 1H), 4.98 (s, 2H), 4.71 (d, J = 5.8 Hz, 2H), 4.38-4.26 (m, 4H), 4.23 (d, J = 5.8 Hz, |
| | | 2H), 3.71-3.65 (m, 2H), 2.92 (s, 3H), 2.39-2.30 (m, 1H), 0.91-0.78 (m, 2H), 0.64- |
| | | 0.47 (m, 2H) ppm |
| 783 | 564.3 | 1H NMR (400 MHz, DMSO-d6) δ = 9.56-9.55 (m, 1H), 9.12 (s, 1H), 8.50 (d, J = 1.6 |
| | | Hz, 1H), 8.38 (br s, 1H), 8.32 (d, J = 9.0 Hz, 1H), 8.23 (d, J = 7.8 Hz, 1H), 7.81 (s, |
| | | 1H), 7.72 (d, J = 7.8 Hz, 1H), 7.45 (s, 1H), 7.21 (d, J = 9.0 Hz, 1H), |
| | | 4.97 (s, 2H), 4.72 (d, J = 5.8 Hz, 2H), 4.25-4.20 (m, 2H), 4.00 (br t, J = 6.1 Hz, |
| | | 2H), 3.91 (s, 3H), 3.69-3.65 (m, 2H), 2.72-2.70 (m, 2H), 1.93-1.90 (m, 2H) ppm |
| 784 | 572.3 | 1H NMR (400 MHz, DMSO-d6) δ = 9.56-9.55 (m, 1H), 9.06 (s, 1H), 8.51 (d, J = 1.8 |
| | | Hz, 1H), 8.33-8.19 (m, 3H), 7.72 (d, J = 7.8 Hz, 1H), 7.61 (s, 1H), 7.46 (s, 1H), |
| | | 4.97 (s, 2H), 4.72 (d, J = 5.8 Hz, 2H), 4.38-4.27 (m, 4H), 4.22-4.10 (m, 2H), 3.71- |
| | | 3.63 (m, 2H), 2.31-2.23 (m, 3H), 1.89-1.78 (m, 1H), 0.95-0.85 (m, 2H), 0.67- |
| | | 0.59 (m, 2H) ppm |
| 785 | 557.3 | 1H NMR (400 MHz, DMSO-d6) δ = 9.60-9.59 (m, 1H), 9.12 (s, 1H), 8.52 (d, J = |
| | | 1.6 Hz, 1H), 8.43 (s, 1H), 8.32-8.22 (m, 2H), 8.17 (s, 1H), 7.84 (d, J = 9.2 Hz, 1H), |
| | | 7.73 (d, J = 7.6 Hz, 1H), 7.51 (s, 1H), 4.97 (s, 2H), 4.74 (d, J = 5.6 Hz, 2H), 4.27- |
| | | 4.19 (m, 2H), 4.18-4.10 (m, 2H), 3.74-3.63 (m, 2H), 2.95-2.91 (m, 2H), 2.12- |
| | | 1.99 (m, 3H), 0.98-0.89 (m, 2H), 0.80-0.78 (m, 2H) ppm |
| 786 | 556.3 | 1H NMR (400 MHz, DMSO-d6) δ 9.53 (s, 1H), 9.15 (s, 1H), 8.54 (d, J = 1.9 Hz, |
| | | 1H), 8.33 (s, 1H), 8.27 (dd, J = 7.8, 1.9 Hz, 1H), 8.07 (d, J = 9.0 Hz, 1H), 7.75 |
| | | (d, J = 7.8 Hz, 1H), 7.63 (dd, J = 8.9, 2.3 Hz, 1H), 7.61 (s, 1H), 7.56 (d, J = 2.2 Hz, |
| | | 1H), 6.87-6.78 (m, 2H), 6.59 (dd, J = 8.4, 2.1 Hz, 1H), 5.01 (s, 2H), 4.73 (d, J = |
| | | 5.7 Hz, 2H), 4.30-4.22 (m, 4H), 3.89-3.82 (m, 2H), 3.73-3.66 (m, 2H), 1.78 |
| | | (ddd, J = 13.4, 8.5, 5.0 Hz, 1H), 0.92-0.78 (m, 2H), 0.54-0.45 (m, 2H). |
| 787 | 557.2 | 1H NMR (400 MHz, DMSO-d6) δ 9.54 (d, J = 6.0 Hz, 1H), 9.16 (s, 1H), 8.56 (d, J = |
| | | 1.9 Hz, 1H), 8.28 (dd, J = 7.8, 1.9 Hz, 1H), 8.01 (d, J = 8.8 Hz, 1H), 7.90 (dd, J = |
| | | 9.1. 2.1 Hz, 1H), 7.76 (d, J = 7.8 Hz, 1H), 7.68 (s, 1H), 7.62 (d, J = 2.3 Hz, 2H), |
| | | 6.90 (d, J = 2.1 Hz, 1H), 6.53 (s, 1H), 5.01 (s, 2H), 4.75 (d, J = 5.8 Hz, 2H), 4.36 |
| | | (t, J = 4.5 Hz, 2H), 4.30-4.23 (m, 2H), 4.04-3.97 (m, 2H), 3.73-3.66 (m, 2H), |
| | | 1.88 (t, J = 8.5 Hz, 1H), 0.96-0.88 (m, 2H), 0.71-0.63 (m, 2H). |
| 788 | 556.2 | 1H NMR (400 MHz, METHANOL-d4) δ = 9.33 (s, 1H), 8.70-8.65 (m, 1H), 8.64- |
| | | 8.58 (m, 2H), 8.41 (d, J = 8.0 Hz, 1H), 8.23-8.20 (m, 1H), 7.98 (s, 1H), 7.74 (d, J = |
| | | 8.4 Hz, 1H), 7.65 (d, J = 8.0 Hz, 1H), 5.05 (s, 2H), 4.94 (s, 2H), 4.38-4.31 (m, 2H), |
| | | 4.05 (s, 2H), 3.55-3.51 (m, 2H), 3.09-3.08 (m, 2H), 3.03-3.02 (m, 2H), 2.07- |
| | | 2.01 (m, 1H), 0.71-0.65 (m, 2H), 0.64-0.58 (m, 2H) ppm |
| 789 | 580.3 | 1H NMR (400 MHz, DMSO-d6) δ = 13.26-12.32 (m, 1H), 9.52-9.50 (m, 1H), 9.05 |
| | | (s, 1H), 8.50 (d, J = 1.6 Hz, 1H), 8.32-8.26 (m, 1H), 8.28-8.22 (m, 1H), 8.08- |
| | | 7.85 (m, 3H), 7.71 (d, J = 8.0 Hz, 1H), 7.57-7.50 (m, 2H), 7.46 (d, J = 2.0 Hz, 1H), |
| | | 7.39 (d, J = 2.0 Hz, 1H), 7.28-7.26 (m, 1H), 7.02 (d, J = 8.4 Hz, 1H), 4.96 (s, 2H), |
| | | 4.68 (br d, J = 5.6 Hz, 2H), 4.24-4.19 (m, 2H), 3.73-3.70 (m, 2H), 3.68-3.64 (m, |
| | | 2H), 2.78-2.75 (m, 2H), 2.00-1.93 (m, 2H) ppm |
| 790 | 560.2 | 1H NMR (400 MHz, DMSO-d6) δ = 9.54-9.50 (m, 1H), 9.07 (s, 1H), 8.51 (d, J = 1.8 |
| | | Hz, 1H), 8.24 (d, J = 7.8 Hz, 1H), 7.95-7.89 (m, 1H), 7.88-7.82 (m, 1H), 7.72 (d, |
| | | J = 7.8 Hz, 1H), 7.52 (s, 2H), 7.38 (d, J = 2.4 Hz, 1H), 6.74 (d, J = 2.4 Hz, 1H), 4.97 |
| | | (s, 2H), 4.69 (d, J = 5.8 Hz, 2H), 4.34-4.18 (m, 4H), 3.97-3.89 (m, 2H), 3.71- |
| | | 3.62 (m, 2H), 2.82 (s, 6H) ppm |
| 791 | 621.3 | 1H NMR (400 MHz, DMSO-d6) δ = 9.84-9.83 (m, 1H), 9.43 (d, J = 2.0 Hz, 1H), 9.41 |
| | | (s, 1H), 8.74 (d, J = 2.0 Hz, 1H), 8.70-8.60 (m, 2H), 8.36 (br s, 1H), 7.91 (d, J = |
| | | 7.4 Hz, 1H), 7.87 (s, 1H), 7.75 (d, J = 8.4 Hz, 1H), 7.03 (d, J = 8.4 Hz, |
| | | 1H), 4.84 (d, J = 5.6 Hz, 2H), 4.32 (br d, J = 11.0 Hz, 2H), 3.81-3.73 (m, 1H), 3.70- |
| | | 3.63 (m, 3H), 3.53 (br dd, J = 13.6 Hz, 1H), 2.52-2.52 (m, 3H), 2.45-2.41 (m, |
| | | 1H), 1.39-1.28 (m, 1H), 1.21 (d, J = 6.2 Hz, 6H) ppm |
| 792 | 559.32 | ¹H NMR (400 MHz, DMSO-d₆) δ 9.51 (t, J = 5.9 Hz, 1H), 8.95 (s, 1H), 8.51 (d, J = |
| | | 1.9 Hz, 1H), 8.24 (dd, J = 7.8, 1.9 Hz, 1H), 8.11 (d, J = 9.2 Hz, 1H), 7.72 (d, J = 7.8 |
| | | Hz, 1H), 7.36 (s, 1H), 7.31 (d, J = 9.2 Hz, 1H), 6.94 (s, 1H), 6.84 (s, 1H), 5.99 (s, |
| | | 2H), 4.97 (s, 2H), 4.69 (d, J = 5.8 Hz, 2H), 4.26-4.19 (m, 2H), 3.95 (t, J = 6.5 Hz, |
| | | 2H), 3.71-3.64 (m, 2H), 2.61 (t, J = 6.6 Hz, 2H), 1.87 (p, J = 6.5 Hz, 2H). |
| 793 | 573.32 | ¹H NMR (400 MHz, DMSO-d₆) δ 9.52 (t, J = 5.9 Hz, 1H), 8.94 (s, 1H), 8.52 (d, J = |
| | | 1.8 Hz, 1H), 8.24 (dd, J = 7.8, 1.9 Hz, 1H), 8.11 (d, J = 9.2 Hz, 1H), 7.72 (d, J = 7.8 |
| | | Hz, 1H), 7.42-7.30 (m, 2H), 6.84 (s, 1H), 6.75 (s, 1H), 4.97 (s, 2H), 4.69 (d, J = |
| | | 5.8 Hz, 2H), 4.22 (d, J = 5.3 Hz, 5H), 3.94 (t, J = 6.4 Hz, 2H), 3.75-3.62 (m, 2H), |
| | | 2.60 (t, J = 6.6 Hz, 2H), 1.87 (p, J = 6.5 Hz, 2H). |
| 794 | 613.2 | 1H NMR (400 MHz, DMSO-d6) δ = 9.56-9.53 (m, 1H), 9.11 (s, 1H), 8.49 (d, J = |
| | | 2.0 Hz, 1H), 8.44 (d, J = 1.2 Hz, 1H), 8.36 (d, J = 8.8 Hz, 1H), 8.23-8.21 (m, 1H), |
| | | 7.72 (d, J = 8.0 Hz, 1H), 7.43 (s, 1H), 7.24-7.23 (m, 1H), 6.95 (d, J = 1.6 Hz, 1H), |
| | | 4.97 (s, 2H), 4.71 (d, J = 5.6 Hz, 2H), 4.48 (s, 2H), 4.31-4.18 (m, 6H), 3.71-3.65 |
| | | (m, 2H), 3.36 (s, 3H) ppm |
| 795 | 612.2 | 1H NMR (400 MHz, DMSO-d6) δ = 9.61-9.58 (m, 1H), 9.13 (s, 1H), 8.52 (d, J = |
| | | 1.6 Hz, 1H), 8.33 (d, J = 9.2 Hz, 1H), 8.25 (d, J = 1.6 Hz, 1H), 8.11 (d, J = 9.2 Hz, |
| | | 1H), 7.73 (d, J = 7.8 Hz, 1H), 7.52 (s, 1H), 7.47 (d, J = 8.0 Hz, 1H), 7.35 (d, J = 8.0 |
| | | Hz, 1H), 4.97 (s, 2H), 4.74 (d, J = 6.0 Hz, 2H), 4.52-4.33 (m, 3H), 4.26-4.15 (m, |
| | | 3H), 3.72-3.64 (m, 2H), 2.63-2.56 (m, 2H) ppm |
| 796 | 579.2 | 1H NMR (400 MHz, DMSO-d6) δ = 9.55-9.45 (m, 1H), 9.07 (s, 1H), 8.50 (d, J = |
| | | 1.6 Hz, 1H), 8.42 (br s, 1H), 8.31 (d, J = 9.2 Hz, 1H), 8.22 (d, J = 7.8 Hz, 1H), 7.73- |
| | | 7.70 (m, 1H), 7.41 (s, 1H), 7.12 (d, J = 9.2 Hz, 1H), 6.85-6.77 (m, |
| | | 2H), 4.96 (s, 2H), 4.70 (d, J = 5.6 Hz, 2H), 4.38 (s, 2H), 4.27-4.20 (m, 6H), 3.69- |
| | | 3.65 (m, 2H), 3.30 (s, 3H) ppm |
| 797 | 590.3 | 1H NMR (400 MHz, METHANOL-d4) δ = 9.19 (s, 1H), 8.57-8.50 (m, 2H), 8.50- |
| | | 8.43 (m, 1H), 8.11 (d, J = 7.8 Hz, 1H), 7.85 (s, 1H), 7.80 (d, J = 7.6 Hz, 1H), 7.61- |
| | | 7.50 (m, 1H), 7.54 (d, J = 7.6 Hz, 1H), 6.48 (d, J = 8.2 Hz, 1H), 4.95 (s, 2H), 4.82 |
| | | (s, 2H), 4.80 (br s, 1H), 4.74 (d, J = 8.4 Hz, 1H), 4.66 (d, J = 19.2 Hz, 2H), 4.26- |
| | | 4.22 (m, 2H), 4.19-4.00 (m, 2H), 3.96 (d, J = 8.4 Hz, 2H), 3.47-3.40 (m, 2H), 3.30- |
| | | 3.23 (m, 1H) ppm |
| 798 | 602.3 | 1H NMR (400 MHz, DMSO-d6) δ = 9.54-9.51 (m, 1H), 9.09 (s, 1H), 8.51 (d, J = |
| | | 1.6 Hz, 1H), 8.24 (d, J = 1.6 Hz, 1H), 7.98-7.91 (m, 1H), 7.88-7.83 (m, 1H), 7.72 |
| | | (d, J = 8.0 Hz, 1H), 7.59-7.53 (m, 2H), 7.50 (d, J = 2.4 Hz, 1H), 6.94 (d, J = 2.4 |
| | | Hz, 1H), 4.97 (s, 2H), 4.70 (d, J = 5.6 Hz, 2H), 4.36-4.27 (m, 2H), 4.26-4.18 (m, |
| | | 2H), 3.99-3.91 (m, 2H), 3.76-3.69 (m, 4H), 3.68-3.63 (m, 2H), 3.07-3.01 (m, 4H) ppm |
| 799 | 567.2 | 1H NMR (400 MHz, DMSO-d6) δ = 9.62-9.44 (m, 1H), 9.18 (s, 1H), 8.51 (d, J = |
| | | 1.8 Hz, 1H), 8.29-8.20 (m, 1H), 8.10-7.97 (m, 1H), 7.91 (d, J = 9.2 Hz, 1H), 7.81- |
| | | 7.67 (m, 2H), 7.62 (s, 1H), 7.32 (d, J = 7.8 Hz, 1H), 7.10 (d, J = 8.0 Hz, 1H), 6.70- |
| | | 6.45 (m, 1H), 4.97 (s, 2H), 4.73 (d, J = 5.6 Hz, 2H), 4.50-4.35 (m, 2H), 4.29-4.18 |
| | | (m, 2H), 4.09-3.99 (m, 2H), 3.73-3.59 (m, 2H) ppm |
| 800 | 558.2 | 1H NMR (400 MHz, DMSO-d6) δ = 9.53-9.44 (m, 1H), 9.39-9.30 (m, 1H), 8.51 |
| | | (d, J = 1.6 Hz, 1H), 8.26-8.18 (m, 1H), 7.97 (d, J = 1.2 Hz, 2H), 7.76-7.69 (m, |
| | | 1H), 7.66-7.58 (m, 2H), 6.90 (d, J = 2.0 Hz, 1H), 4.97 (s, 2H), 4.79 (d, J = 5.6 Hz, |
| | | 2H), 4.37-4.29 (m, 2H), 4.24-4.20 (m, 2H), 4.05-3.98 (m, 2H), 3.69-3.64 (m, |
| | | 2H), 1.92-1.80 (m, 1H), 0.94-0.86 (m, 2H), 0.69-0.60 (m, 2H) ppm |
| 801 | 558.3 | 1H NMR (400 MHz, DMSO-d6) δ = 9.60-9.55 (m, 1H), 9.29 (d, J = 2.4 Hz, 1H), 9.21 |
| | | (s, 1H), 8.53 (d, J = 1.6 Hz, 1H), 8.40 (br s, 1H), 8.25 (d, J = 7.8 Hz, 1H), 7.99 (d, |
| | | J = 2.4 Hz, 1H), 7.73 (d, J = 7.8 Hz, 1H), 7.67 (s, 1H), 7.60 (d, J = 2.0 |
| | | Hz, 1H), 6.90 (d, J = 2.0 Hz, 1H), 4.97 (s, 2H), 4.75 (d, J = 5.6 Hz, 2H), 4.41-4.34 |
| | | (m, 2H), 4.25-4.20 (m, 2H), 4.00-3.94 (m, 2H), 3.69-3.64 (m, 2H), 1.89-1.80 |
| | | (m, 1H), 0.93-0.86 (m, 2H), 0.68-0.61 (m, 2H) ppm |
| 802 | 564.2 | 1H NMR (400 MHz, DMSO-d6) δ = 9.59-9.58 (m, 1H), 9.12 (s, 1H), 8.52 (d, J = |
| | | 1.6 Hz, 1H), 8.26-8.18 (m, 2H), 8.15 (d, J = 3.6 Hz, 1H), 7.88 (d, J = 8.8 Hz, 1H), |
| | | 7.73 (d, J = 8.0 Hz, 1H), 7.55 (s, 1H), 4.98 (s, 2H), 4.75 (d, J = 5.6 Hz, 2H), 4.26- |
| | | 4.20 (m, 2H), 4.17-4.12 (m, 2H), 4.09 (d, J = 4.0 Hz, 3H), 3.70-3.66 (m, 2H), 2.76- |
| | | 2.73 (m, 2H), 1.96-1.88 (m, 2H) ppm |
| 803 | 561.3 | 1H NMR (400 MHz, DMSO-d6) δ = 9.60-9.49 (m, 1H), 8.99 (s, 1H), 8.51 (d, J = |
| | | 1.6 Hz, 1H), 8.28-8.19 (m, 3H), 7.72 (d, J = 7.6 Hz, 1H), 7.48 (d, J = 9.2 Hz, 1H), |
| | | 7.36 (s, 1H), 6.16 (s, 1H), 5.00-4.94 (m, 2H), 4.73-4.64 (m, 2H), 4.31-4.19 (m, |
| | | 6H), 3.73-3.62 (m, 2H), 2.98 (s, 6H) ppm |
| 804 | 576.3 | 1H NMR (400 MHz, DMSO-d6) δ = 9.60-9.57 (m, 1H), 9.13 (s, 1H), 8.52 (d, J = |
| | | 1.6 Hz, 1H), 8.45 (s, 1H), 8.33 (d, J = 9.2 Hz, 1H), 8.26-8.23 (m, 1H), 8.17 (d, J = |
| | | 9.2 Hz, 1H), 7.73 (d, J = 8.0 Hz, 1H), 7.51 (s, 1H), 7.05 (d, J = 11.2 Hz, 1H), 4.98 |
| | | (s, 2H), 4.74 (d, J = 5.6 Hz, 2H), 4.38-4.34 (m, 2H), 4.33-4.28 (m, 2H), 4.25- |
| | | 4.20 (m, 2H), 3.71-3.65 (m, 2H), 2.03-1.97 (m, 1H), 0.90-0.87 (m, 2H), 0.83- |
| | | 0.78 (m, 2H) ppm |
| 805 | 593.2 | 1H NMR (400 MHz, DMSO-d6) δ = 9.54-9.51 (m, 1H), 9.10 (s, 1H), 8.50 (d, J = |
| | | 2.0 Hz, 1H), 8.42 (s, 1H), 8.34 (d, J = 8.8 Hz, 1H), 8.23-8.21 (m, 1H), 7.71 (d, J = |
| | | 7.6 Hz, 1H), 7.44 (s, 1H), 7.26-7.22 (m, 1H), 6.43-6.41 (m, 1H), 4.97 (s, 2H), 4.71 |
| | | (d, J = 5.6 Hz, 2H), 4.30-4.14 (m, 6H), 3.70-3.59 (m, 2H), 2.08-2.02 (m, 1H), |
| | | 1.02-0.95 (m, 2H), 0.79-0.74 (m, 2H) ppm |
| 806 | 530.1 | 1H NMR (400 MHz, DMSO-d6) δ = 9.64-9.54(m, 1H), 9.31 (s, 1H), 8.85 (s, 1H), |
| | | 8.58 (d, J = 8.6 Hz, 1H), 8.54 (d, J = 1.6 Hz, 1H), 8.26 (d, J = 7.8 Hz, 1H), 8.07 (d, |
| | | J = 8.6 Hz, 1H), 7.84-7.51 (m, 3H), 4.98 (s, 2H), 4.79 (d, J = 5.6 Hz, 2H), 4.28-4.19 |
| | | (m, 2H), 4.06 (s, 3H), 3.73-3.65 (m, 2H) ppm |
| 807 | 574.3 | 1H NMR (400 MHz, DMSO-d6) δ = 9.61-9.58 (m, 1H), 9.14 (s, 1H), 8.53 (d, J = 1.6 |
| | | Hz, 1H), 8.35 (s, 2H), 8.26-8.16 (m, 1H), 7.74 (d, J = 7.8 Hz, 1H), 7.53 (s, 1H), |
| | | 7.47 (d, J = 8.2 Hz, 1H), 7.33 (d, J = 8.1 Hz, 1H), 7.04-6.66 (m, 1H), |
| | | 4.75 (d, J = 5.7 Hz, 2H), 4.39 (br d, J = 8.6 Hz, 4H) ppm |
| 808 | 491.2 | 1H NMR (400 MHz, DMSO-d6) δ = 9.64 (br d, J = 5.6 Hz, 1H), 9.40 (s, 1H), 8.61 (d, |
| | | J = 8.6 Hz, 1H), 8.53 (d, J = 1.6 Hz, 1H), 8.36-8.24 (m, 3H), 8.18 (d, J = 8.6 Hz, |
| | | 1H), 7.79 (s, 1H), 7.73 (d, J = 7.8 Hz, 1H), 7.19-7.10 (m, 1H), 4.97 (s, 2H), 4.82 (br |
| | | d, J = 5.6 Hz, 2H), 4.26-4.19 (m, 2H), 3.98 (s, 3H), 3.71-3.64 (m, 2H) ppm |
| 809 | 572.3 | 1H NMR (400 MHz, DMSO-d6) δ = 9.60-9.57 (m, 1H), 9.10 (s, 1H), 8.52 (d, J = |
| | | 1.6 Hz, 1H), 8.42 (br s, 1H), 8.30 (d, J = 9.2 Hz, 1H), 8.26-8.24 (m, 1H), 8.22- |
| | | 8.18 (m, 1H), 7.73 (d, J = 8.0 Hz, 1H), 7.48 (s, 1H), 7.19 (d, J = 8.0 Hz, 1H), 6.95 |
| | | (d, J = 8.0 Hz, 1H), 4.97 (s, 2H), 4.73 (br d, J = 5.6 Hz, 2H), 4.33-4.20 (m, 6H), |
| | | 3.73-3.65 (m, 2H), 1.81-1.64 (m, 1H), 1.20-1.08 (m, 4H), 0.99-0.97 (m, 1H), |
| | | 0.73-0.63 (m, 1H) ppm |
| 810 | 572.3 | 1H NMR (400 MHz, DMSO-d6) δ = 9.60-9.57 (m, 1H), 9.10 (s, 1H), 8.52 (s, 1H), |
| | | 8.42 (br s, 1H), 8.30 (d, J = 9.2 Hz, 1H), 8.25 (br d, J = 7.6 Hz, 1H), 8.22-8.17 (m, |
| | | 1H), 7.73 (d, J = 7.6 Hz, 1H), 7.48 (s, 1H), 7.19 (d, J = 8.0 Hz, 1H), 6.95 (d, J = 8.0 |
| | | Hz, 1H), 4.97 (s, 2H), 4.73 (br d, J = 5.6 Hz, 2H), 4.28 (br s, 4H), 4.25-4.20 (m, |
| | | 2H), 3.71-3.66 (m, 2H), 1.79-1.68 (m, 1H), 1.20-1.08 (m, 4H), 1.02-0.93 (m, |
| | | 1H), 0.68-0.66 (m, 1H) ppm |
| 811 | 558.3 | 1H NMR (400 MHz, METHANOL-d4) δ = 9.26 (s, 1H), 9.22 (s, 1H), 8.59 (d, J = 2.0 |
| | | Hz, 1H), 8.53-8.44 (m, 1H), 8.22-8.12 (m, 2H), 7.72 (d, J = 2.0 Hz, 1H), 7.62 (d, |
| | | J = 7.6 Hz, 1H), 7.57 (s, 1H), 6.95 (d, J = 2.0 Hz, 1H), 5.04 (s, 2H), 4.81 (s, 2H), 4.37- |
| | | 4.30 (m, 6H), 3.54-3.51 (m, 2H), 1.96-1.85 (m, 1H), 1.02-0.92 (m, 2H), 0.74- |
| | | 0.63 (m, 2H) ppm |
| 812 | 530.2 | 1H NMR (400 MHz, DMSO-d6) δ = 9.64-9.61 (m, 1H), 9.27 (s, 1H), 8.62-8.48 (m, |
| | | 3H), 8.37 (s, 1H), 8.26-8.24 (m, 1H), 7.88 (d, J = 8.4 Hz, 1H), 7.74 (d, J = 8.0 Hz, |
| | | 1H), 7.69-7.25 (m, 2H), 4.98 (s, 2H), 4.78 (d, J = 6.0 Hz, 2H), 4.26-4.20 (m, 2H), |
| | | 3.83 (s, 3H), 3.69-3.66 (m, 2H) ppm |
| 813 | 576.3 | 1H NMR (400 MHz, DMSO-d6) δ = 9.56-9.45 (m, 1H), 9.07 (s, 1H), 8.53-8.46 (m, |
| | | 2H), 8.32 (d, J = 9.0 Hz, 1H), 8.23-8.20 (m, 1H), 7.72 (d, J = 7.8 Hz, 1H), 7.54 (d, |
| | | J = 9.2 Hz, 1H), 7.42 (s, 1H), 4.97 (s, 2H), 4.71 (d, J = 5.6 Hz, 2H), 4.43-4.40 (m, |
| | | 2H), 4.31-4.14 (m, 4H), 3.71-3.64 (m, 2H), 2.23-2.20 (m, 1H), 1.05-0.89 (m, 4H) ppm |
| 814 | 571.3 | 1H NMR (400 MHz, DMSO-d6) δ = 9.62-9.50 (m, 1H), 9.05 (br s, 1H), 8.52 (s, |
| | | 1H), 8.36-8.01 (m, 2H), 7.85-7.69 (m, 3H), 7.47 (br s, 1H), 6.17 (br d, J = 6.0 Hz, |
| | | 1H), 4.97 (s, 2H), 4.72 (br d, J = 5.6 Hz, 2H), 4.28-4.00 (m, 8H), 3.73-3.66 (m, |
| | | 2H), 2.69 (br s, 2H), 2.31-2.22 (m, 2H), 1.93-1.82 (m, 2H) ppm |
| 815 | 530.3 | 1H NMR (400 MHz, DMSO-d6) δ = 9.64-9.60 (m, 1H), 9.37 (s, 1H), 8.63-8.49 (m, |
| | | 2H), 8.41 (br s, 1H), 8.25 (br d, J = 7.8 Hz, 1H), 8.16 (d, J = 8.6 Hz, 1H), 7.86-7.75 |
| | | (m, 2H), 7.73 (d, J = 7.8 Hz, 1H), 7.46 (br t, J = 7.8 Hz, 1H), 7.17 (d, J = 8.4 Hz, |
| | | 1H), 7.08-7.00 (m, 1H), 4.97 (s, 2H), 4.81 (br d, J = 5.6 Hz, 2H), 4.29-4.12 (m, |
| | | 2H), 3.96 (d, J = 6.8 Hz, 2H), 3.72-3.63 (m, 2H), 1.29-1.14 (m, 1H), 0.52 (br d, |
| | | J = 6.8 Hz, 2H), 0.31 (br d, J = 5.0 Hz, 2H) ppm |
| 816 | 574.3 | 1H NMR (400 MHz, DMSO-d6) δ = 9.62-9.51 (m, 1H), 9.04 (s, 1H), 8.52 (d, J = |
| | | 1.6 Hz, 1H), 8.30-8.21 (m, 1H), 8.13 (d, J = 9.2 Hz, 1H), 7.90 (d, J = 9.2 Hz, 1H), |
| | | 7.73 (d, J = 7.6 Hz, 1H), 7.46 (s, 1H), 6.97 (s, 1H), 4.97 (s, 2H), 4.72 (d, J = 5.6 Hz, |
| | | 2H), 4.32 (s, 2H), 4.32-4.21 (m, 2H), 4.18-4.09 (m, 2H), 3.71-3.65 (m, 2H), 3.30- |
| | | 3.29 (m, 3H), 2.75-2.70 (m, 2H), 2.26 (s, 3H), 2.00-1.92 (m, 2H) ppm |
| 817 | 637.2 | 1H NMR (400 MHz, DMSO-d6) δ = 9.63-9.53 (m, 1H), 9.04 (s, 1H), 8.55-8.50 (m, |
| | | 1H), 8.30-8.21 (m, 3H), 7.77-7.71 (m, 1H), 7.61 (d, J = 2.4 Hz, 1H), 7.44 (s, 1H), |
| | | 6.94 (d, J = 2.4 Hz, 1H), 5.02-4.94 (m, 2H), 4.75-4.70 (m, 2H), 4.37-4.28 (m, |
| | | 4H), 4.26-4.20 (m, 2H), 3.97-3.83 (m, 1H), 3.73-3.63 (m, 2H), 3.05-2.90 (m, |
| | | 2H), 2.80 (s, 3H), 2.68-2.60 (m, 2H) ppm |
| 818 | 574.3 | 1H NMR (400 MHz, DMSO-d6) δ = 9.53-9.50 (m, 1H), 9.14 (s, 1H), 8.46 (d, J = |
| | | 2.4 Hz, 1H), 8.35 (s, 2H), 8.26-8.24 (m, 1H), 7.50 (s, 1H), 7.47 (d, J = 8.0 Hz, 1H), |
| | | 7.39 (d, J = 8.4 Hz, 1H), 7.33 (d, J = 8.4 Hz, 1H), 7.02-6.70 (m, 1H), 4.73 (d, J = |
| | | 5.6 Hz, 2H), 4.45-4.34 (m, 4H) ppm |
| 819 | 601.3 | 1H NMR (400 MHz, DMSO-d6) δ = 9.60-9.48 (m, 1H), 9.05-8.95 (m, 1H), 8.56- |
| | | 8.48 (m, 1H), 8.44-8.38 (m, 1H), 8.28-8.15 (m, 3H), 7.73 (d, J = 7.8 Hz, 1H), 7.56 |
| | | (d, J = 2.4 Hz, 1H), 7.45-7.40 (m, 1H), 6.83-6.75 (m, 1H), 5.01-4.94 (m, 2H), |
| | | 4.75-4.68 (m, 2H), 4.36-4.17 (m, 6H), 4.04-3.95 (m, 1H), 3.72-3.64 (m, 2H), |
| | | 2.80-2.76 (m, 3H), 2.25-2.14 (m, 2H), 2.09-1.98 (m, 2H), 1.71-1.58 (m, 2H) ppm |
| 820 | 530.2 | 1H NMR (400 MHz, DMSO-d6) δ = 9.64-9.61 (m, 1H), 9.38 (s, 1H), 8.64 (d, J = |
| | | 8.8 Hz, 1H), 8.53 (d, J = 1.2 Hz, 1H), 8.49-8.45 (m, 1H), 8.33-8.22 (m, 2H), 7.88- |
| | | 7.79 (m, 3H), 7.73 (d, J = 7.6 Hz, 1H), 7.47-7.43 (m, 1H), 7.11-7.09 (m, 1H), 4.98 |
| | | (s, 2H), 4.81 (d, J = 5.6 Hz, 2H), 4.30-4.15 (m, 2H), 3.93 (d, J = 6.8 Hz, 2H), 3.75- |
| | | 3.61 (m, 2H), 1.30-1.20 (m, 1H), 0.64-0.55 (m, 2H), 0.37-0.34 (m, 2H) ppm |
| 821 | 560.2 | 1H NMR (400 MHz, DMSO-d6) δ = 9.55-9.52 (m, 1H), 9.05 (s, 1H), 8.64 (s, 1H), |
| | | 8.51 (d, J = 1.6 Hz, 1H), 8.28-8.20 (m, 2H), 8.13 (s, 1H), 7.72 (d, J = 7.6 Hz, 1H), |
| | | 7.47 (d, J = 9.2 Hz, 1H), 7.41 (s, 1H), 7.29 (s, 1H), 4.97 (s, 2H), 4.71 (d, J = 5.6 Hz, |
| | | 2H), 4.45 (s, 2H), 4.25-4.19 (m, 2H), 4.03-4.00 (m, 2H), 3.72-3.63 (m, 2H), 3.37 |
| | | (s, 3H), 2.82-2.79 (m, 2H), 1.97-1.90 (m, 2H) ppm |
| 822 | 558.3 | 1H NMR (400 MHz, DMSO-d6) δ = 9.57 (br t, J = 5.9 Hz, 1H), 9.23 (s, 1H), 8.52 (d, |
| | | J = 1.5 Hz, 1H), 8.24 (dd, J = 1.5. 7.8 Hz, |
| | | 1H), 8.07 (d, J = 9.0 Hz, 1H), 7.97-7.81 (m, 3H), 7.72 (d, J = 7.8 Hz, 1H), 7.66 (s, |
| | | 1H), 4.97 (s, 2H), 4.74 (br d, J = 5.9 Hz, |
| | | 2H), 4.39-4.31 (m, 2H), 4.24-4.19 (m, 2H), 4.08-3.99 (m, 2H), 3.69-3.62 (m, |
| | | 2H), 1.91-1.83 (m, 1H), 0.83-0.74 (m, 2H), 0.74-0.66 (m, 2H) |
| 823 | 580.3 | 1H NMR (400 MHz, DMSO-d6) δ = 9.59-9.56 (m, 1H), 9.10 (s, 1H), 8.53 (d, J = 1.6 |
| | | Hz, 1H), 8.44 (br s, 1H), 8.30-8.18 (m, 2H), 7.90 (d, J = 9.2 Hz, 1H), 7.79-7.64 |
| | | (m, 2H), 7.50 (s, 1H), 7.16 (d, J = 7.6 Hz, 1H), 5.95-5.60 (m, 1H), 4.98 (s, 2H), |
| | | 4.94-4.69 (m, 4H), 4.29-4.19 (m, 2H), 4.14-4.10 (m, 2H), 3.70-3.64 (m, 2H), |
| | | 2.85-2.83 (m, 2H), 1.97 (br t, J = 6.0 Hz, 2H) ppm |
| 824 | 505.2 | 1H NMR (400 MHz, DMSO-d6) δ = 9.62-9.61 (m, 1H), 9.31 (s, 1H), 8.69 (d, J = |
| | | 8.8 Hz, 1H), 8.56-8.47 (m, 2H), 8.38 (d, J = 7.6 Hz, 1H), 8.27-8.25 (m, 1H), 7.80- |
| | | 7.65 (m, 2H), 6.40 (d, J = 7.6 Hz, 1H), 4.97 (s, 2H), 4.79 (d, J = 5.6 Hz, 2H), 4.27- |
| | | 4.15 (m, 2H), 3.72-3.64 (m, 2H), 3.56 (s, 3H), 2.47 (s, 3H) ppm |
| 825 | 564.3 | 1H NMR (400 MHz, DMSO-d6) δ = 9.54-9.50 (m, 1H), 9.12 (s, 1H), 8.50 (d, J = 1.6 |
| | | Hz, 1H), 8.48-8.45 (m, 1H), 8.35 (d, J = 9.2 Hz, 1H), 8.22-8.18 (m, 1H), 7.94 (d, |
| | | J = 2.8 Hz, 1H), 7.71 (d, J = 7.8 Hz, 1H), 7.40 (s, 1H), 7.29-7.27 (m, 1H), 4.96 (s, |
| | | 2H), 4.71 (d, J = 5.6 Hz, 2H), 4.28-4.16 (m, 2H), 4.05 (br d, J = 3.6 Hz, 2H), 3.89 |
| | | (s, 3H), 3.73-3.60 (m, 2H), 2.69-2.61 (m, 2H), 1.91-1.81 (m, 2H) ppm |
| 826 | 491.2 | 1H NMR (400 MHz, METHANOL-d4) δ = 9.31 (s, 1H), 8.61 (d, J = 2.0 Hz, 1H), 8.54- |
| | | 8.48 (m, 2H), 8.38-8.37 (m, 1H), 8.22-8.20 (m, 1H), 7.92 (s, 1H), 7.88-7.87 (m, |
| | | 1H), 7.64 (d, J = 7.8 Hz, 1H), 6.58-6.55 (m, 1H), 5.05 (s, 2H), 4.92 (s, 2H), 4.37- |
| | | 4.31 (m, 2H), 3.69 (s, 3H), 3.55-3.51 (m, 2H) ppm |
| 827 | 576.2 | 1H NMR (400 MHz, DMSO-d6) δ = 9.58-9.55 (m, 1H), 9.12 (s, 1H), 8.52 (d, J = |
| | | 2.0 Hz, 1H), 8.34-8.22 (m, 3H), 8.13 (s, 1H), 7.73 (d, J = 8.0 Hz, 1H), 7.66 (d, J = |
| | | 8.8 Hz, 1H), 7.52 (s, 1H), 4.98 (s, 2H), 4.74 (d, J = 5.6 Hz, 2H), 4.46-4.33 (m, 4H), |
| | | 4.28-4.18 (m, 2H), 3.76-3.58 (m, 2H), 1.96-1.90 (m, 1H), 1.00-0.93 (m, 2H), |
| | | 0.82-0.77 (m, 2H) ppm |
| 828 | 608.3 | 1H NMR (400 MHz, DMSO-d6) δ = 9.59-9.58 (m, 1H), 9.14 (s, 1H), 8.52 (d, J = 1.6 |
| | | Hz, 1H), 8.46-8.21 (m, 4H), 7.73 (d, J = 7.8 Hz, 1H), 7.51 (s, 1H), 7.28 (d, J = 7.8 |
| | | Hz, 1H), 6.99 (d, J = 8.0 Hz, 1H), 4.97 (s, 2H), 4.74 (br d, J = 6.0 Hz, 2H), 4.33 (br |
| | | d, J = 3.2 Hz, 4H), 4.26-4.18 (m, 2H), 3.72-3.65 (m, 2H), 3.46-3.44 (m, 1H), |
| | | 2.97-2.74 (m, 4H) ppm |
| 829 | 574.3 | 1H NMR (400 MHz, DMSO-d6) δ = 9.55-9.52 (m, 1H), 8.98 (s, 1H), 8.51 (d, J = |
| | | 1.6 Hz, 1H), 8.38 (s, 1H), 8.29-8.20 (m, 2H), 8.10 (d, J = 9.2 Hz, 1H), 7.72 (d, J = |
| | | 7.6 Hz, 1H), 7.41 (s, 1H), 7.18 (s, 1H), 7.05-6.89 (m, 2H), 4.97 (s, 2H), 4.71 (d, J = |
| | | 6.0 Hz, 2H), 4.33-4.30 (m, 2H), 4.25-4.19 (m, 2H), 3.69-3.64 (m, 2H), 3.62- |
| | | 3.59 (m, 2H), 3.18 (s, 3H), 2.04-1.86 (m, 1H), 1.11-1.02 (m, 2H), 0.86-0.77 (m, 2H) ppm |
| 830 | 577.1 | 1H NMR (400 MHz, METHANOL-d4) δ = 9.09-8.98 (m, 1H), 8.63-8.57 (m, 1H), |
| | | 8.47-8.37 (m, 1H), 8.26-8.14 (m, 2H), 7.95-7.88 (m, 1H), 7.66-7.59 (m, 2H), |
| | | 7.19-7.12 (m, 1H), 5.06-5.02 (m, 4H), 4.35-4.32 (m, 2H), 4.08-4.02 (m, 2H), |
| | | 3.54-3.50 (m, 2H), 2.85-2.82 (m, 6H), 2.79-2.75 (m, 2H), 2.07-1.97 (m, 2H) ppm |
| 831 | 572.3 | 1H NMR (400 MHz, DMSO-d6) δ = 9.59-9.58 (m, 1H), 9.10 (s, 1H), 8.52 (d, J = |
| | | 2.0 Hz, 1H), 8.44 (s, 1H), 8.36-8.19 (m, 3H), 7.73 (d, J = 7.6 Hz, 1H), 7.49 (s, 1H), |
| | | 7.24 (d, J = 8.4 Hz, 1H), 7.00 (d, J = 8.0 Hz, 1H), 4.97 (s, 2H), 4.73 (d, J = 5.6 Hz, |
| | | 2H), 4.41-4.14 (m, 6H), 3.74-3.64 (m, 2H), 2.14-2.08 (m, 1H), 1.28-1.11 (m, |
| | | 1H), 0.97-0.94 (m, 1H), 0.87-0.83 (m, 4H) ppm |
| 832 | 572.3 | 1H NMR (400 MHz, DMSO-d6) δ = 9.60-9.57 (m, 1H), 9.10 (s, 1H), 8.52 (d, J = |
| | | 2.0 Hz, 1H), 8.42 (s, 1H), 8.35-8.19 (m, 3H), 7.73 (d, J = 7.6 Hz, 1H), 7.49 (s, 1H), |
| | | 7.24 (d, J = 8.0 Hz, 1H), 7.00 (d, J = 8.4 Hz, 1H), 4.97 (s, 2H), 4.73 (d, J = 5.6 Hz, |
| | | 2H), 4.41-4.13 (m, 6H), 3.73-3.65 (m, 2H), 2.14-2.08 (m, 1H), 1.30-1.11 (m, |
| | | 1H), 0.98-0.95 (m, 1H), 0.87-0.85 (m, 4H) ppm |
| 833 | 574.3 | 1H NMR (400 MHz, DMSO-d6) δ = 9.54-9.53 (m, 1H), 8.97 (s, 1H), 8.51 (d, J = 1.6 |
| | | Hz, 1H), 8.40 (br s, 1H), 8.30 (d, J = 2.4 Hz, 1H), 8.24-8.23 (m, 1H), 8.08 (d, J = 9.2 |
| | | Hz, 1H), 7.72 (d, J = 7.8 Hz, 1H), 7.52-7.50 (m, 1H), 7.41 (s, 1H), 7.33 (d, J = 8.4 |
| | | Hz, 1H), 6.96 (d, J = 9.4 Hz, 1H), 4.97 (s, 2H), 4.70 (br d, J = 5.8 Hz, 2H), 4.31 (br t, |
| | | J = 6.0 Hz, 2H), 4.26-4.19 (m, 2H), 3.70-3.65 (m, 2H), 3.60 (t, J = 6.0 Hz, 2H), |
| | | 3.18 (s, 3H), 2.05-1.91 (m, 1H), 1.08-0.96 (m, 2H), 0.83-0.71 (m, 2H) ppm |
| 834 | 573.3 | 1H NMR (400 MHz, DMSO-d6) δ = 9.57-9.52 (m, 1H), 9.03-8.97 (m, 1H), 8.51 (s, |
| | | 1H), 8.27-8.19 (m, 3H), 7.75-7.69 (m, 1H), 7.50-7.43 (m, 1H), 7.38-7.34 (m, |
| | | 1H), 5.90 (s, 1H), 4.97 (s, 2H), 4.69 (br d, J = 5.6 Hz, 2H), 4.31-4.27 (m, 2H), 4.25- |
| | | 4.20 (m, 4H), 3.92-3.85 (m, 4H), 3.72-3.64 (m, 2H), 2.32-2.25 (m, 2H) ppm |
| 835 | 610.2 | 1H NMR (400 MHz, DMSO-d6) δ = 9.63-9.54 (m, 1H), 9.14-9.07 (m, 1H), 8.56- |
| | | 8.48 (m, 1H), 8.43-8.37 (m, 1H), 8.27-8.21 (m, 2H), 8.08-8.00 (m, 1H), 7.77- |
| | | 7.70 (m, 2H), 7.51 (s, 1H), 7.12-6.75 (m, 1H), 5.01-4.94 (m, 2H), 4.78-4.70 (m, |
| | | 2H), 4.54 (s, 2H), 4.24-4.20 (m, 2H), 4.20-4.15 (m, 2H), 3.70-3.66 (m, 2H), 3.50 |
| | | (br s, 3H), 2.91-2.87 (m, 2H), 2.02-1.95 (m, 2H) ppm |
| 836 | 559.3 | 1H NMR (400 MHz, METHANOL-d4) δ = 9.03 (s, 1H), 8.60 (d, J = 2.0 Hz, 1H), 8.41 |
| | | (d, J = 3.2 Hz, 1H), 8.26-8.18 (m, 2H), 7.88 (d, J = 5.6 Hz, 1H), 7.66-7.61 (m, |
| | | 2H), 7.52 (d, J = 9.2 Hz, 1H), 6.96 (d, J = 6.0 Hz, 1H), 5.04 (s, 2H), 4.95 (d, J = 3.6 |
| | | Hz, 2H), 4.34-4.32 (m, 2H), 4.06-4.03 (m, 2H), 3.55-3.50 (m, 2H), 2.90 (s, 6H), |
| | | 2.79-2.73 (m, 2H), 2.03-1.98 (m, 2H) ppm |
| 837 | 559.3 | 1H NMR (400 MHz, DMSO-d6) δ = 9.58-9.57 (m, 1H), 9.02 (s, 1H), 8.53 (d, J = |
| | | 1.6 Hz, 1H), 8.31 (s, 1H), 8.24 (d, J = 1.6 Hz, 1H), 8.09 (d, J = 9.2 Hz, 1H), 7.98 (d, |
| | | J = 5.6 Hz, 1H), 7.76-7.73 (m, 2H), 7.46 (s, 1H), 6.70 (d, J = 5.6 Hz, 1H), 4.98 (s, |
| | | 2H), 4.72 (br d, J = 5.6 Hz, 2H), 4.26-4.20 (m, 2H), 4.05-4.02 (m, 2H), 3.70-3.67 |
| | | (m, 2H), 2.78 (s, 6H), 2.67 (br d, J = 2.0 Hz, 2H), 1.93-1.77 (m, 2H) ppm |
| 838 | 582.2 | 1H NMR (400 MHz, DMSO-d6) δ = 9.60-9.55 (m, 1H), 9.14 (s, 1H), 8.53 (s, 1H), |
| | | 8.34 (s, 1H), 8.25 (d, J = 9.4 Hz, 2H), 7.73 (d, J = 7.9 Hz, 1H), 7.52 (s, 1H), 7.42 (d, |
| | | J = 8.1 Hz, 1H), 7.20 (d, J = 8.1 Hz, 1H), 6.06-5.52 (m, 1H), 5.01-4.96 (m, 2H), |
| | | 4.95-4.77 (m, 2H), 4.75 (br d, J = 5.9 Hz, 2H), 4.41-4.29 (m, 4H), 4.25-4.18 (m, |
| | | 2H), 3.70-3.66 (m, 2H) ppm |
| 839 | 575.1 | 1H NMR (400 MHz, DMSO-d6) δ = 9.56 (d, J = 5.6 Hz, 1H), 9.20 (s, 1H), 8.50 (d, |
| | | J = 1.8 Hz, 1H), 8.40 (s, 1H), 8.36 (m, 2H), 8.23 (m, 1H), 7.72 (d, J = 7.8 Hz, 1H), |
| | | 7.63 (d, J = 9.0 Hz, 1H), 7.56 (s, 1H), 7.08 (d, J = 6.1 Hz, 1H), 4.97 (s, 2H), 4.75 (d, |
| | | J = 5.8 Hz, 2H), 4.43 (d, J = 6.1 Hz, 2H), 4.26-4.19 (m, 2H), 3.70-3.59 (m, 4H), |
| | | 3.17 (s, 3H), 2.10-2.04 (m, 1H), 1.01-0.92 (m, 4H) ppm |
| 840 | 574.3 | 1H NMR (400 MHz, METHANOL-d4) δ = 8.90 (s, 1H), 8.60 (d, J = 1.6 Hz, 1H), 8.20- |
| | | 8.19 (m, 1H), 8.02 (d, J = 9.2 Hz, 1H), 7.69-7.68 (m, 1H), 7.63 (d, J = 8.0 Hz, |
| | | 1H), 7.58 (s, 1H), 7.16 (d, J = 8.0 Hz, 1H), 7.11 (d, J = 7.6 Hz, 1H), 7.03 (d, J = 9.2 |
| | | Hz, 1H), 5.04 (s, 2H), 4.82 (s, 2H), 4.40-4.38 (m, 2H), 4.37-4.32 (m, 2H), 3.72- |
| | | 3.70 (m, 2H), 3.56-3.50 (m, 2H), 3.28 (s, 3H), 2.12-1.99 (m, 1H), 1.00-0.92 (m, 4H) ppm |
| 841 | 545.3 | 1H NMR (400 MHz, DMSO-d6) δ = 9.59-9.58 (m, 1H), 9.16 (s, 1H), 8.52 (d, J = 1.6 |
| | | Hz, 1H), 8.35-8.19 (m, 2H), 8.08 (d, J = 9.0 Hz, 1H), 7.73 (d, J = 7.8 Hz, 1H), 7.54 |
| | | (s, 1H), 4.97 (s, 2H), 4.75 (d, J = 5.7 Hz, 2H), 4.29-4.16 (m, 2H), 4.14-4.03 (m, |
| | | 2H), 3.73-3.63 (m, 2H), 2.71 (br t, J = 6.5 Hz, 2H), 2.34 (d, J = 16.0 Hz, 6H), 2.05- |
| | | 1.92 (m, 2H) ppm |
| 842 | 534.3 | 1H NMR (400 MHz, DMSO-d6) δ = 9.63-9.53 (m, 1H), 9.09 (s, 1H), 8.53 (d, J = |
| | | 1.8 Hz, 1H), 8.43 (s, 1H), 8.28-8.17 (m, 3H), 7.91 (d, J = 9.2 Hz, 1H), 7.76-7.71 |
| | | (m, 1H), 7.68 (d, J = 6.6 Hz, 1H), 7.51 (s, 1H), 7.12 (dd, J = 4.8, 7.4 Hz, 1H), 5.55- |
| | | 5.28 (m, 1H), 4.98 (s, 2H), 4.87-4.77 (m, 1H), 4.74 (d, J = 5.7 Hz, 2H), 4.30-4.14 |
| | | (m, 2H), 4.03-3.85 (m, 1H), 3.75-3.61 (m, 2H), 3.44-3.41 (m, 1H), 3.13 (br dd, |
| | | J = 2.4, 3.5 Hz, 1H) ppm |
| 843 | 558.2 | 1H NMR (400 MHz, DMSO-d6) δ = 9.56 (s, 1H), 9.05 (s, 1H), 8.51 (d, J = 2.0 Hz, |
| | | 1H), 8.38 (s, 1H), 8.28 (d, J = 8.8 Hz, 1H), 8.24-8.22 (m, 1H), 7.84 (d, J = 8.4 Hz, |
| | | 1H), 7.72 (d, J = 7.6 Hz, 1H), 7.47 (d, J = 9.2 Hz, 1H), 7.40 (s, 1H), 6.93 (d, J = 8.4 |
| | | Hz, 1H), 4.97 (s, 2H), 4.70 (d, J = 6.0 Hz, 2H), 4.40-4.30 (m, 2H), 4.26-4.14 (m, |
| | | 4H), 3.70-3.64 (m, 2H), 2.05-1.95 (m, 1H), 0.88-0.87 (m, 2H), 0.83-0.81 (m, 2H) ppm |
| 844 | 558.2 | 1H NMR (400 MHz, DMSO-d6) δ = 9.59-9.40 (m, 1H), 9.09 (s, 1H), 8.93 (s, 1H), |
| | | 8.51 (d, J = 1.6 Hz, 1H), 8.24 (m, 1H), 7.73 (d, J = 7.8 Hz, 1H), 7.49 (d, J = 8.2 Hz, |
| | | 1H), 7.44 (s, 1H), 6.72-6.62 (m, 2H), 4.97 (s, 2H), 4.73 (br d, |
| | | J = 5.6 Hz, 2H), 4.32 (d, J = 4.6 Hz, 2H), 4.27-4.11 (m, 4H), 3.75-3.62 (m, 2H), |
| | | 1.94-1.83 (m, 1H), 0.98-0.88 (m, 2H), 0.70-0.60 (m, 2H) ppm |
| 845 | 561.3 | 1H NMR (400 MHz, DMSO-d6) δ = 9.56-9.53 (m, 1H), 8.98 (s, 1H), 8.51 (d, J = |
| | | 1.6 Hz, 1H), 8.41-8.34 (m, 1H), 8.26-8.18 (m, 2H), 7.73-7.68 (m, 2H), 7.42- |
| | | 7.29 (m, 2H), 6.28 (d, J = 8.8 Hz, 1H), 4.97 (s, 2H), 4.69 (d, J = 6.0 Hz, 2H), 4.34- |
| | | 4.28 (m, 2H), 4.24-4.21 (m, 2H), 4.19-4.16 (m, 2H), 3.71-3.66 (m, 2H), 2.98 (s, 6H) ppm |
| 846 | 568.2 | 1H NMR (400 MHz, DMSO-d6) δ = 9.59-9.56 (m, 1H), 9.13 (s, 1H), 8.50 (d, J = |
| | | 1.6 Hz, 1H), 8.41 (br s, 1H), 8.38 (d, J = 9.2 Hz, 1H), 8.27-8.17 (m, 2H), 7.72 (d, |
| | | J = 8.0 Hz, 1H), 7.59 (d, J = 9.2 Hz, 1H), 7.47 (s, 1H), 7.28 (d, J = 8.4 Hz, 1H), 6.95- |
| | | 6.67 (m, 1H), 4.97 (s, 2H), 4.73 (d, J = 6.0 Hz, 2H), 4.52-4.44 (m, 2H), 4.23-4.21 |
| | | (m, 4H), 3.70-3.63 (m, 2H) ppm |
| 847 | 608.4 | 1H NMR (400 MHz, DMSO-d6) δ = 9.59 (m, 1H), 9.11 (s, 1H), 8.53 (s, 1H), 8.30 (s, |
| | | 2H), 8.25-8.23 (m, 1H), 7.91 (d, J = 1.8 Hz, 1H), 7.73 (d, J = 7.8 Hz, 1H), 7.50 (s, |
| | | 1H), 7.31 (d, J = 1.8 Hz, 1H), 6.06-5.66 (m, 1H), 4.97 (s, 2H), 4.74 (br d, J = 5.6 |
| | | Hz, 2H), 4.34 (br s, 4H), 4.26-4.17 (m, 2H), 3.74-3.64 (m, 2H), 1.18-1.09 (m, |
| | | 2H), 1.02 (br s, 2H) ppm |
| 848 | 531.3 | 1H NMR (400 MHz, DMSO-d6) δ = 9.66-9.56 (m, 1H), 9.18 (s, 1H), 8.56-8.49 (m, |
| | | 1H), 8.44-8.41 (m, 2H), 8.34-8.30 (m, 1H), 8.27-8.22 (m, 1H), 8.06-8.00 (m, |
| | | 1H), 7.76-7.70 (m, 1H), 7.56 (s, 1H), 5.02-4.94 (m, 2H), 4.75 (br d, J = 5.6 Hz, |
| | | 2H), 4.28-4.18 (m, 2H), 4.15-4.08 (m, 2H), 3.71-3.66 (m, 2H), 2.77 (br t, J = 6.4 |
| | | Hz, 2H), 2.37 (s, 3H), 2.07-1.97 (m, 2H) ppm |
| 849 | 527.2 | 1H NMR (400 MHz, DMSO-d6) δ = 9.68-9.65 (m, 1H), 9.43 (s, 1H), 8.75-8.60 (m, |
| | | 4H), 8.54 (s, 1H), 8.35 (s, 1H), 8.26 (d, J = 7.6 Hz, 1H), 7.90-7.82 (m, 2H), 7.74 (d, |
| | | J = 7.6 Hz, 1H), 7.68-7.25 (m, 1H), 4.98 (s, 2H), 4.83 (d, J = 5.6 Hz, 2H), 4.30- |
| | | 4.17 (m, 2H), 3.75-3.66 (m, 2H) ppm |
| 850 | 491.2 | 1H NMR (400 MHz, DMSO-d6) δ = 9.65 (d, J = 5.8 Hz, 1H), 9.38 (s, 1H), 8.67- |
| | | 8.62 (m, 1H), 8.62-8.56 (m, 2H), 8.54 (d, J = 1.6 Hz, 1H), 8.48 (d, J = 2.8 Hz, 1H), |
| | | 8.27 (m, 1H), 7.81 (s, 1H), 7.74 (d, J = 7.8 Hz, 1H), 7.58 (m, 1H), 4.98 (s, 2H), 4.82 |
| | | (br d, J = 5.6 Hz, 2H), 4.28-4.18 (m, 2H), 3.93 (s, 3H), 3.74-3.64 (m, 2H) ppm |
| 851 | 517.3 | 1H NMR (400 MHz, DMSO-d6) δ = 9.63-9.60 (m, 1H), 9.38 (s, 1H), 8.68-8.63 (m, |
| | | 1H), 8.62-8.57 (m, 2H), 8.54 (d, J = 2.0 Hz, 2H), 8.27 (br d, J = 7.8 Hz, 1H), 7.81 |
| | | (s, 1H), 7.77-7.66 (m, 2H), 4.98 (s, 2H), 4.82 (br d, J = 5.6 Hz, 2H), 4.29-4.16 (m, |
| | | 2H), 4.08 (td, J = 2.9, 5.9 Hz, 1H), 3.73-3.60 (m, 2H), 0.92-0.84 (m, 2H), 0.79- |
| | | 0.69 (m, 2H) ppm |
| 852 | 572.3 | 1H NMR (400 MHz, DMSO-d6) δ = 9.75-9.72 (m, 1H), 9.42 (s, 1H), 8.62 (d, J = |
| | | 9.2 Hz, 1H), 8.51 (d, J = 1.6 Hz, 1H), 8.28-8.25 (m, 1H), 8.14 (s, 1H), 8.07 (s, 1H), |
| | | 7.85 (d, J = 9.2 Hz, 1H), 7.78-7.69 (m, 2H), 7.31 (s, 1H), 4.98 (s, 2H), 4.82 (d, J = |
| | | 5.6 Hz, 2H), 4.25-4.20 (m, 2H), 4.09-4.04 (m, 3H), 3.69-3.65 (m, 2H), 2.85- |
| | | 2.75 (m, 2H), 2.03-2.00 (m, 2H), 0.74-0.68 (m, 2H), 0.66-0.65 (m, 2H) ppm |
| 853 | 576.3 | 1H NMR (400 MHz, DMSO-d6) δ = 9.57 (m, 1H), 9.16 (s, 1H), 8.50 (s, 1H), 8.46 (br |
| | | s, 1H), 8.42 (d, J = 9.0 Hz, 1H), 8.23 (m, 1H), 8.01 (s, 1H), 7.72 (d, J = 7.8 Hz, 1H), |
| | | 7.48 (s, 1H), 7.29 (m, 1H), 4.97 (s, 2H), 4.73 (m, 2H), 4.29-4.21 (m, 6H), 3.69- |
| | | 3.65 (m, 2H), 2.17 (m, 1H), 0.97-0.87 (m, 4H) ppm |
| 854 | 582.3 | 1H NMR (400 MHz, DMSO-d6) δ = 9.59 (t, J = 5.9 Hz, 1H), 9.14 (s, 1H), 8.53 (d, |
| | | J = 1.8 Hz, 1H), 8.41-8.32 (m, 1H), 8.29-8.20 (m, 2H), 7.73 (d, J = 7.8 Hz, 1H), |
| | | 7.52 (s, 1H), 7.42 (d, J = 8.1 Hz, 1H), 7.20 (d, J = 8.1 Hz, 1H), 5.99-5.61 (m, 1H), |
| | | 5.04-4.96 (m, 2H), 4.95-4.67 (m, 4H), 4.47-4.28 (m, 4H), 4.23 (br dd, J = 4.1, |
| | | 5.7 Hz, 2H), 3.72-3.63 (m, 2H) ppm |
| 855 | 527.9 | 1H NMR (400 MHz, DMSO-d6) δ = 9.58 (m, 1H), 9.03 (s, 1H), 8.53 (s, 1H), 8.36 (br |
| | | s, 1H), 8.26 (m, 1H), 8.15-8.08 (m, 2H), 7.85 (m, 1H), 7.73 (d, J = 8.2 Hz, 1H), |
| | | 7.55 (d, J = 9.4 Hz, 1H), 7.48 (s, 1H), 7.11 (m, 1H), 5.20 (m, 1H), 4.98 (s, 2H), 4.73 |
| | | (d, J = 6.4 Hz, 2H), 4.26-4.19 (m, 2H), 3.71-3.66 (m, 2H), 3.19 (d, J = 11.6 Hz, |
| | | 1H), 2.18-2.11 (m, 1H), 2.11-2.03 (m, 1H), 1.01 (m, 1H), 0.80 (m, 1H) ppm |
| 856 | 582.3 | 1H NMR (400 MHz, DMSO-d6) δ = 9.59 (m, 1H), 9.14 (s, 1H), 8.53 (d, J = 1.8 Hz, |
| | | 1H), 8.40 (s, 1H), 8.37-8.33 (m, 1H), 8.31-8.22 (m, 2H), 7.73 (d, J = 7.8 Hz, 1H), |
| | | 7.52 (s, 1H), 7.42 (d, J = 8.1 Hz, 1H), 7.21 (d, J = 8.2 Hz, 1H), 5.94-5.65 (m, 1H), |
| | | 4.98 (s, 2H), 4.92-4.72 (m, 4H), 4.38 (s, 4H), 4.25-4.21 (m, 2H), 3.70-3.66 (m, 2H) ppm |

### Example 5. Assay for ATPase Catalytic Activity of BRM and BRG-1

The ATPase catalytic activity of BRM or BRG-1 was measured by an in vitro biochemical assay using ADP-Glo™ (Promega, V9102). The ADP-Glo™ kinase assay is performed in two steps once the reaction is complete. The first step is to deplete any unconsumed ATP in the reaction. The second step is to convert the reaction product ADP to ATP, which will be utilized by the luciferase to generate luminesce and be detected by a luminescence reader, such as Envision.

The assay reaction mixture (10 μL) contains 30 nM of BRM or BRG-1, 20 nM salmon sperm DNA (from Invitrogen, UfraPure™ Salmon Sperm DNA Solution, cat #15632011), and 400 μM of ATP in the ATPase assay buffer, which comprises of 20 mM Tris, pH 8, 20 mM MgCl₂, 50 mM NaCl, 0.1% Tween-20, and 1 mM fresh DTT (Piercer™ DTT (Dithiothreitol), cat #20290). The reaction is initiated by the addition of the 2.5 μL ATPase solution to 2.5 μL ATP/DNA solution on low volume white Proxiplate-384 plus plate (PerkinElmer, cat #6008280) and incubates at room temperature for 1 hour. Then, following addition of 5 μL of ADP-Glo™ Reagent provided in the kit, the reaction incubates at room temperature for 40 minutes. Then, 10 μL of Kinase Detection Reagent provided in the kit is added to convert ADP to ATP, and the reaction incubates at room temperature for 60 minutes. Finally, luminescence measurement is collected with a plate-reading luminometer, such as Envision.

BRM and BRG-1 were synthesized from high five insect cell lines with a purity of greater than 90%. IC₅₀ data from the ATPase catalytic activity assay described herein are shown in Tables 5A and 5B below.

TABLE 5A
BRM and BRG-1 Inhibition Data
for Compounds of the Invention

| | BRM | BRG1 | |
|---|---|---|---|
| cpd | IC₅₀ | IC₅₀ | |
| # | (μM) | (μM) | Ratio* |
| 13 | 0.0090 | 0.0345 | 3.83 |
| 15 | 0.0075 | 0.0511 | 6.80 |
| 37 | 0.0086 | 0.0850 | 9.89 |
| 40 | 0.0089 | 0.0884 | 9.95 |
| 41 | 0.0181 | 0.1936 | 10.70 |
| 42 | 0.0173 | 0.1272 | 7.37 |
| 43 | 0.0198 | 0.1848 | 9.33 |
| 44 | 0.0905 | 0.6944 | 7.67 |
| 45 | 0.0051 | 0.0198 | 3.87 |
| 46 | 0.0093 | 0.0459 | 4.92 |
| 47 | 0.0745 | 0.4566 | 6.12 |
| 48 | 0.2942 | 1.8418 | 6.26 |
| 49 | 0.0248 | 0.4553 | 18.33 |
| 50 | 0.1465 | 1.5178 | 10.36 |
| 51 | 0.0349 | 0.2000 | 5.74 |
| 52 | 0.0760 | 0.7165 | 9.43 |
| 53 | 0.0117 | 0.1165 | 9.98 |
| 54 | 0.0095 | 0.0447 | 4.70 |
| 55 | 0.0253 | 0.2392 | 9.45 |
| 56 | 0.0136 | 0.2401 | 17.62 |
| 57 | 0.0080 | 0.1181 | 14.73 |
| 58 | 0.0969 | 0.5459 | 5.63 |
| 59 | 0.0730 | 0.7798 | 10.68 |
| 60 | 0.7885 | 5.0000 | 6.34 |
| 61 | 0.9565 | 5.0000 | 5.23 |
| 62 | 0.0189 | 0.2467 | 13.08 |
| 63 | 0.0214 | 0.6332 | 29.53 |
| 64 | 0.0175 | 0.3399 | 19.39 |
| 65 | 0.0162 | 0.0997 | 6.14 |
| 66 | 0.2106 | 1.6384 | 7.78 |
| 67 | 0.0894 | 0.5855 | 6.55 |
| 68 | 0.4330 | 4.9257 | 11.38 |
| 69 | 0.2622 | 2.4747 | 9.44 |
| 70 | 0.0368 | 0.4059 | 11.02 |
| 71 | 0.0239 | 0.2848 | 11.93 |
| 72 | 0.2241 | 1.2277 | 5.48 |
| 73 | 0.0068 | 0.0794 | 11.74 |
| 74 | 0.1223 | 1.1775 | 9.63 |
| 75 | 0.0346 | 0.6806 | 19.65 |
| 76 | 0.0457 | 0.6818 | 14.90 |
| 77 | 0.0775 | 1.0738 | 13.85 |
| 78 | 0.0190 | 0.1978 | 10.42 |
| 79 | 0.0146 | 0.1155 | 7.89 |
| 80 | 0.1761 | 2.0698 | 11.75 |
| 81 | 0.2750 | 1.7419 | 6.33 |
| 82 | 0.4271 | 5.0000 | 11.71 |
| 83 | 0.0081 | 0.1347 | 16.64 |
| 84 | 0.1210 | 0.5701 | 4.71 |
| 85 | 0.0157 | 0.1286 | 8.19 |
| 86 | 0.0225 | 0.3244 | 14.43 |
| 87 | 0.6887 | 4.9159 | 7.14 |
| 88 | 0.0342 | 0.5347 | 15.63 |
| 89 | 0.0308 | 0.6479 | 21.02 |
| 90 | 0.1660 | 2.1405 | 12.89 |
| 91 | 0.0048 | 0.0170 | 3.52 |
| 92 | 0.0077 | 0.1030 | 13.36 |
| 93 | 0.0096 | 0.0936 | 9.74 |
| 94 | 0.0320 | 0.2883 | 9.02 |
| 95 | 0.0407 | 0.6101 | 15.00 |
| 96 | 0.0497 | 0.6999 | 14.09 |
| 97 | 0.0203 | 0.3245 | 15.97 |
| 98 | 0.0330 | 0.2573 | 7.80 |
| 99 | 0.0787 | 0.8841 | 11.24 |
| 100 | 0.0080 | 0.0941 | 11.83 |
| 101 | 0.0079 | 0.0577 | 7.32 |
| 102 | 0.0187 | 0.1434 | 7.68 |
| 103 | 0.2525 | 2.7681 | 10.96 |
| 104 | 0.2027 | 0.9026 | 4.45 |
| 105 | 0.0699 | 0.9371 | 13.41 |
| 106 | 0.0761 | 0.6667 | 8.76 |
| 107 | 0.0056 | 0.0560 | 9.98 |
| 108 | 0.6725 | 5.0000 | 7.43 |
| 109 | 0.0169 | 0.1274 | 7.52 |
| 110 | 0.0209 | 0.4068 | 19.51 |
| 111 | 0.0060 | 0.0274 | 4.58 |
| 112 | 0.0129 | 0.1763 | 13.71 |
| 113 | 0.0595 | 0.5832 | 9.79 |
| 114 | 0.0708 | 1.6108 | 22.76 |
| 115 | 0.0235 | 0.2364 | 10.05 |
| 116 | 0.0226 | 0.2361 | 10.43 |
| 117 | 0.0446 | 0.3125 | 7.01 |
| 118 | 0.0249 | 0.2516 | 10.11 |
| 119 | 0.0400 | 0.2211 | 5.53 |
| 120 | 0.0447 | 0.4690 | 10.48 |
| 121 | 0.0867 | 0.4974 | 5.73 |
| 122 | 0.0103 | 0.0677 | 6.56 |
| 123 | 0.0232 | 0.2902 | 12.51 |
| 124 | 0.0378 | 0.5405 | 14.29 |
| 125 | 0.0183 | 0.2240 | 12.26 |
| 126 | 0.0335 | 0.2952 | 8.80 |
| 127 | 0.8458 | 4.0512 | 4.79 |
| 128 | 0.0426 | 0.4681 | 10.99 |
| 129 | 0.0479 | 0.5703 | 11.91 |
| 130 | 0.0107 | 0.1043 | 9.77 |
| 131 | 0.0716 | 0.5096 | 7.12 |
| 132 | 0.0125 | 0.2044 | 16.30 |
| 133 | 0.0615 | 0.4866 | 7.91 |
| 134 | 0.1856 | 2.4647 | 13.28 |
| 135 | 0.1119 | 1.0599 | 9.48 |
| 136 | 0.0334 | 0.2387 | 7.15 |
| 137 | 0.0225 | 0.3824 | 16.99 |
| 138 | 0.0108 | 0.0507 | 4.70 |
| 139 | 0.4318 | 2.0748 | 4.81 |
| 140 | 0.0352 | 0.2433 | 6.92 |
| 141 | 0.0130 | 0.1690 | 12.97 |
| 142 | 0.2302 | 2.6598 | 11.55 |
| 143 | 0.0076 | 0.1085 | 14.36 |
| 144 | 0.1466 | 1.3496 | 9.21 |
| 145 | 0.0036 | 0.0571 | 15.77 |
| 146 | 0.0153 | 0.1972 | 12.92 |
| 147 | 0.0156 | 0.3938 | 25.22 |
| 148 | 0.4547 | 3.0912 | 6.80 |
| 149 | 0.0366 | 0.8104 | 22.13 |
| 150 | 0.0338 | 0.3963 | 11.71 |
| 151 | 0.0608 | 0.6693 | 11.00 |
| 152 | 0.1357 | 2.1401 | 15.77 |
| 153 | 0.0463 | 0.4512 | 9.74 |
| 154 | 0.1424 | 2.1276 | 14.94 |
| 155 | 0.0045 | 0.0252 | 5.59 |
| 156 | 0.3659 | 1.9193 | 5.25 |
| 157 | 0.0145 | 0.3195 | 21.99 |
| 158 | 0.0131 | 0.2617 | 20.03 |
| 159 | 0.0367 | 0.4091 | 11.15 |
| 160 | 0.0030 | 0.0136 | 4.54 |
| 161 | 0.0047 | 0.0452 | 9.62 |
| 162 | 0.0384 | 0.5639 | 14.67 |
| 163 | 0.0208 | 0.2639 | 12.71 |
| 164 | 0.1102 | 1.4106 | 12.80 |
| 165 | 0.1155 | 2.1237 | 18.39 |
| 166 | 0.0168 | 0.2063 | 12.29 |
| 167 | 0.0593 | 0.8271 | 13.95 |
| 168 | 0.0940 | 0.8788 | 9.35 |
| 169 | 0.0030 | 0.0099 | 3.33 |
| 170 | 0.1090 | 0.6894 | 6.33 |
| 171 | 0.0524 | 0.4993 | 9.52 |
| 172 | 0.0092 | 0.1055 | 11.42 |
| 173 | 0.1080 | 1.4875 | 13.78 |
| 174 | 0.0288 | 0.2657 | 9.24 |
| 175 | 0.0040 | 0.0207 | 5.17 |
| 176 | 0.4370 | 1.7905 | 4.10 |
| 177 | 0.0158 | 0.2223 | 14.02 |
| 178 | 0.1799 | 1.3897 | 7.72 |
| 179 | 0.0333 | 0.3827 | 11.49 |
| 180 | 0.0176 | 0.2717 | 15.48 |
| 181 | 0.0405 | 0.3673 | 9.06 |
| 182 | 0.0445 | 0.4217 | 9.47 |
| 183 | 0.0089 | 0.1014 | 11.33 |
| 184 | 0.0805 | 0.7688 | 9.55 |
| 185 | 0.3566 | 1.3359 | 3.75 |
| 186 | 0.0221 | 0.2539 | 11.50 |
| 187 | 0.1093 | 2.6676 | 24.40 |
| 188 | 0.2871 | 5.0000 | 17.42 |
| 189 | 0.1352 | 0.7511 | 5.56 |
| 190 | 0.0459 | 0.3935 | 8.57 |
| 191 | 0.0601 | 1.1849 | 19.73 |
| 192 | 0.0051 | 0.0327 | 6.45 |
| 193 | 0.0354 | 0.2615 | 7.38 |
| 194 | 0.2193 | 2.7836 | 12.69 |
| 195 | 0.0037 | 0.0313 | 8.49 |
| 196 | 0.2358 | 0.7218 | 3.06 |
| 197 | 0.0193 | 0.2443 | 12.64 |
| 198 | 0.2225 | 0.9667 | 4.34 |
| 199 | 0.2521 | 2.4103 | 9.56 |
| 200 | 0.0081 | 0.0591 | 7.31 |
| 201 | 0.0194 | 0.1768 | 9.11 |
| 202 | 0.0179 | 0.1357 | 7.59 |
| 203 | 0.1540 | 1.5757 | 10.23 |
| 204 | 0.0565 | 0.6789 | 12.01 |
| 205 | 0.0903 | 1.4844 | 16.45 |
| 206 | 0.0080 | 0.1097 | 13.71 |
| 207 | 0.4070 | 5.0000 | 12.29 |
| 208 | 0.0427 | 0.3771 | 8.84 |
| 209 | 0.4979 | 3.6192 | 7.27 |
| 210 | 0.0176 | 0.1742 | 9.92 |
| 211 | 0.0070 | 0.0213 | 3.05 |
| 212 | 0.0143 | 0.1217 | 8.51 |
| 213 | 0.0095 | 0.1319 | 13.94 |
| 214 | 0.3311 | 3.3139 | 10.01 |
| 215 | 0.0070 | 0.0662 | 9.47 |
| 216 | 0.0109 | 0.0937 | 8.63 |
| 217 | 0.0612 | 0.9646 | 15.77 |
| 218 | 0.1656 | 1.6219 | 9.79 |
| 219 | 0.1363 | 1.2336 | 9.05 |
| 220 | 0.0160 | 0.1442 | 9.02 |
| 221 | 0.0823 | 0.7942 | 9.65 |
| 222 | 0.0129 | 0.1360 | 10.58 |
| 223 | 0.0069 | 0.0496 | 7.16 |
| 224 | 0.0259 | 0.3655 | 14.09 |
| 225 | 0.0046 | 0.0287 | 6.29 |
| 226 | 0.0111 | 0.1365 | 12.29 |
| 227 | 0.0504 | 0.4608 | 9.15 |
| 228 | 0.0205 | 0.2823 | 13.77 |
| 229 | 0.1188 | 1.5210 | 12.81 |
| 230 | 0.0416 | 0.3994 | 9.60 |
| 231 | 0.1303 | 2.3431 | 17.98 |
| 232 | 0.0585 | 0.4097 | 7.00 |
| 233 | 0.0771 | 1.0802 | 14.01 |
| 234 | 0.1878 | 1.5198 | 8.09 |
| 235 | 0.1541 | 1.1274 | 7.32 |
| 236 | 0.0340 | 0.4400 | 12.93 |
| 237 | 0.0227 | 0.4681 | 20.63 |
| 238 | 0.2615 | 5.0000 | 19.12 |
| 239 | 0.0206 | 0.4217 | 20.48 |
| 240 | 0.0065 | 0.0238 | 3.66 |
| 241 | 0.0623 | 1.0740 | 17.23 |
| 242 | 0.2862 | 3.1650 | 11.06 |
| 243 | 0.5280 | 4.6594 | 8.82 |
| 244 | 0.0207 | 0.3375 | 16.32 |
| 245 | 0.0187 | 0.1763 | 9.42 |
| 246 | 0.1481 | 1.5234 | 10.29 |
| 247 | 0.3249 | 2.0014 | 6.16 |
| 248 | 0.0702 | 0.5899 | 8.41 |
| 249 | 0.1012 | 1.0560 | 10.44 |
| 250 | 0.1047 | 0.7508 | 7.17 |
| 251 | 0.1757 | 1.7026 | 9.69 |
| 252 | 0.0753 | 1.3786 | 18.31 |
| 253 | 0.0207 | 0.3985 | 19.26 |
| 254 | 0.0542 | 0.5165 | 9.53 |
| 255 | 0.0364 | 0.4867 | 13.37 |
| 256 | 0.0490 | 0.4258 | 8.69 |
| 257 | 0.0154 | 0.1074 | 6.99 |
| 258 | 0.2213 | 4.1234 | 18.63 |
| 259 | 0.4670 | 5.0000 | 10.71 |
| 260 | 0.6974 | 5.0000 | 7.17 |
| 261 | 0.0165 | 0.2326 | 14.10 |
| 262 | 0.4657 | 2.6236 | 5.63 |
| 263 | 0.0233 | 0.3482 | 14.94 |
| 264 | 0.0235 | 0.5903 | 25.10 |
| 265 | 0.1670 | 2.3986 | 14.36 |
| 266 | 0.8186 | 5.0000 | 6.11 |
| 267 | 0.0352 | 1.0920 | 30.99 |
| 268 | 0.0163 | 0.3426 | 21.00 |
| 269 | 0.1998 | 5.0000 | 25.03 |
| 270 | 0.1266 | 1.5747 | 12.44 |
| 271 | 0.0161 | 0.2045 | 12.70 |
| 272 | 0.2447 | 4.2296 | 17.28 |
| 273 | 0.1192 | 2.7980 | 23.47 |
| 274 | 0.0696 | 0.5889 | 8.46 |
| 275 | 0.0849 | 1.3209 | 15.55 |
| 276 | 0.0818 | 0.5842 | 7.14 |
| 277 | 0.6148 | 5.3589 | 8.72 |
| 278 | 0.0738 | 1.9132 | 25.91 |
| 279 | 0.5289 | 5.0000 | 9.45 |
| 280 | 0.2118 | 2.1737 | 10.26 |
| 281 | 0.0828 | 1.6505 | 19.92 |
| 282 | 0.0042 | 0.0193 | 4.64 |
| 283 | 0.1969 | 3.4800 | 17.67 |
| 284 | 0.0121 | 0.1779 | 14.69 |
| 285 | 0.0193 | 0.4967 | 25.69 |
| 286 | 0.3514 | 4.8887 | 13.91 |
| 287 | 0.0242 | 0.5937 | 24.53 |
| 288 | 0.1966 | 3.4572 | 17.58 |
| 289 | 0.0598 | 0.9579 | 16.01 |
| 290 | 0.1725 | 1.2411 | 7.19 |
| 291 | 0.0103 | 0.0972 | 9.47 |
| 292 | 0.6670 | 5.0000 | 7.50 |
| 293 | 0.2226 | 2.4122 | 10.84 |
| 294 | 0.1562 | 2.7134 | 17.37 |
| 295 | 0.3851 | 5.0000 | 12.98 |
| 296 | 0.3156 | 5.0000 | 15.84 |
| 297 | 0.1404 | 2.0314 | 14.47 |
| 298 | 0.2213 | 2.1913 | 9.90 |
| 299 | 0.8687 | 6.3572 | 7.32 |
| 300 | 0.0647 | 0.7328 | 11.32 |
| 301 | 0.8582 | 8.8778 | 10.34 |
| 302 | 0.2767 | 2.3534 | 8.50 |
| 303 | 0.0123 | 0.1557 | 12.63 |
| 304 | 0.0094 | 0.1357 | 14.43 |
| 305 | 0.0300 | 0.2790 | 9.32 |
| 306 | 0.8547 | 4.2542 | 4.98 |
| 307 | 0.2674 | 1.6907 | 6.32 |
| 308 | 0.0054 | 0.0158 | 2.91 |

*Ratio is a numeric value produced by dividing BRG1 IC₅₀ (μM) by BRM IC₅₀ (μM).

TABLE 5B
BRM and BRG-1 Inhibition Data
for Compounds of the Invention

| | BRM | BRG1 | |
|---|---|---|---|
| cpd | IC50 | IC50 | |
| # | (μM) | (μM) | Ratio* |
| 309 | 0.0139 | 0.2509 | 18.06 |
| 310 | 0.0058 | 0.0724 | 12.46 |
| 311 | 0.0220 | 0.2830 | 12.86 |
| 312 | 0.0245 | 0.3262 | 13.30 |
| 313 | 0.1048 | 0.3868 | 3.69 |
| 314 | 0.0457 | 0.4804 | 10.51 |
| 315 | 0.0184 | 0.3405 | 18.47 |
| 316 | 0.0471 | 0.4946 | 10.49 |
| 317 | 0.0063 | 0.0348 | 5.55 |
| 318 | 0.0094 | 0.0804 | 8.53 |
| 319 | 0.0211 | 0.3287 | 15.59 |
| 320 | 0.0120 | 0.2260 | 18.81 |
| 321 | 0.0160 | 0.1953 | 12.19 |
| 322 | 0.0110 | 0.1205 | 10.96 |
| 323 | 0.0171 | 0.2300 | 13.46 |
| 324 | 0.0248 | 0.6135 | 24.73 |
| 325 | 0.0106 | 0.1737 | 16.33 |
| 326 | 0.0099 | 0.0810 | 8.22 |
| 327 | 0.0192 | 0.2908 | 15.15 |
| 328 | 0.8429 | 5.0000 | 5.93 |
| 329 | 0.0086 | 0.1302 | 15.19 |
| 330 | 0.0110 | 0.4727 | 43.09 |
| 331 | 0.0135 | 0.4179 | 30.98 |
| 332 | 0.0166 | 0.2245 | 13.49 |
| 333 | 0.0171 | 0.2270 | 13.29 |
| 334 | 0.0212 | 0.3772 | 17.82 |
| 335 | 0.0243 | 0.2884 | 11.87 |
| 336 | 0.0066 | 0.1067 | 16.08 |
| 337 | 0.0219 | 0.2115 | 9.67 |
| 338 | 0.0201 | 0.2186 | 10.88 |
| 339 | 0.0096 | 0.0858 | 8.89 |
| 340 | 0.0222 | 0.3093 | 13.96 |
| 341 | 0.0251 | 0.1847 | 7.37 |
| 342 | 0.2835 | 4.3205 | 15.24 |
| 343 | 1.3471 | 5.0000 | 3.71 |
| 344 | 0.0076 | 0.0203 | 2.68 |
| 345 | 0.0150 | 0.2511 | 16.77 |
| 346 | 0.3675 | 2.7742 | 7.55 |
| 347 | 1.2297 | 3.7312 | 3.03 |
| 348 | 0.0743 | 2.5579 | 34.42 |
| 349 | 0.0241 | 0.2526 | 10.47 |
| 350 | 0.0081 | 0.1054 | 12.99 |
| 351 | 5.0000 | 5.0000 | 1.00 |
| 352 | 0.0602 | 0.4830 | 8.02 |
| 353 | 0.0095 | 0.1247 | 13.13 |
| 354 | 0.0140 | 0.4149 | 29.57 |
| 355 | 0.0087 | 0.0639 | 7.36 |
| 356 | 3.3085 | 5.0000 | 1.51 |
| 357 | 0.0214 | 0.2931 | 13.72 |
| 358 | 0.0046 | 0.0354 | 7.69 |
| 359 | 5.0000 | 5.0000 | 1.00 |
| 360 | 0.9737 | 5.0000 | 5.14 |
| 361 | 0.0074 | 0.1448 | 19.62 |
| 362 | 0.0169 | 0.2379 | 14.06 |
| 363 | 0.2062 | 5.0000 | 24.25 |
| 364 | 0.0190 | 0.2314 | 12.21 |
| 365 | 0.0131 | 0.3248 | 24.88 |
| 366 | 0.3242 | 2.9626 | 9.14 |
| 367 | 0.0084 | 0.1508 | 17.87 |
| 368 | 5.0000 | 5.0000 | 1.00 |
| 369 | 0.0361 | 0.4155 | 11.52 |
| 370 | 0.0146 | 0.1522 | 10.43 |
| 371 | 0.1965 | 5.0000 | 25.45 |
| 372 | 0.0109 | 0.1173 | 10.78 |
| 373 | 0.0129 | 0.1383 | 10.75 |
| 374 | 0.0088 | 0.0707 | 8.04 |
| 375 | 0.0427 | 0.6296 | 14.74 |
| 376 | 0.0664 | 0.5353 | 8.06 |
| 377 | 0.0124 | 0.1518 | 12.27 |
| 378 | 0.0117 | 0.1619 | 13.88 |
| 379 | 0.2582 | 1.7106 | 6.63 |
| 380 | 0.0982 | 0.5109 | 5.20 |
| 381 | 0.0194 | 0.2409 | 12.43 |
| 382 | 0.0091 | 0.1200 | 13.22 |
| 383 | 0.0066 | 0.0724 | 10.88 |
| 384 | 0.0143 | 0.4073 | 28.41 |
| 386 | 0.0092 | 0.1397 | 15.24 |
| 387 | 0.0683 | 0.7268 | 10.65 |
| 388 | 0.0184 | 0.5874 | 31.86 |
| 389 | 0.0137 | 0.5278 | 38.63 |
| 390 | 0.0607 | 1.0465 | 17.24 |
| 391 | 0.0287 | 0.4560 | 15.87 |
| 392 | 0.0421 | 1.1020 | 26.15 |
| 393 | 0.0190 | 0.6384 | 33.52 |
| 394 | 0.0058 | 0.0879 | 15.21 |
| 395 | 0.0041 | 0.0586 | 14.47 |
| 396 | 0.0059 | 0.1035 | 17.40 |
| 397 | 0.0152 | 0.1750 | 11.51 |
| 398 | 0.1848 | 0.9191 | 4.97 |
| 399 | 0.0078 | 0.1138 | 14.57 |
| 400 | 0.0204 | 0.4784 | 23.51 |
| 401 | 1.3209 | 5.0000 | 3.79 |
| 402 | 0.0057 | 0.0609 | 10.67 |
| 404 | 0.0084 | 0.1424 | 17.03 |
| 405 | 0.0109 | 0.1884 | 17.27 |
| 406 | 0.0050 | 0.0697 | 13.86 |
| 407 | 0.0125 | 0.0586 | 4.69 |
| 408 | 0.4756 | 2.2706 | 4.77 |
| 409 | 0.0166 | 0.1426 | 8.58 |
| 410 | 0.1650 | 1.1019 | 6.68 |
| 411 | 0.0146 | 0.2752 | 18.90 |
| 412 | 0.0765 | 0.6528 | 8.53 |
| 413 | 0.0129 | 0.2098 | 16.27 |
| 414 | 0.0233 | 0.1927 | 8.27 |
| 415 | 0.0206 | 0.4940 | 23.93 |
| 416 | 0.0983 | 2.1252 | 21.63 |
| 417 | 0.2527 | 5.0000 | 19.79 |
| 418 | 0.0136 | 0.1932 | 14.19 |
| 419 | 0.0245 | 0.3837 | 15.63 |
| 420 | 0.0042 | 0.0426 | 10.13 |
| 421 | 0.0568 | 0.8276 | 14.57 |
| 422 | 0.0109 | 0.3102 | 28.45 |
| 423 | 0.0152 | 0.4911 | 32.23 |
| 424 | 0.0208 | 0.6026 | 29.03 |
| 425 | 0.1008 | 0.9657 | 9.58 |
| 426 | 0.0238 | 0.1360 | 5.71 |
| 427 | 0.0473 | 0.4311 | 9.12 |
| 428 | 0.0503 | 0.3241 | 6.45 |
| 429 | 0.0074 | 0.0764 | 10.32 |
| 430 | 0.4769 | 2.4113 | 5.06 |
| 431 | 0.0161 | 0.1516 | 9.43 |
| 432 | 1.4960 | 5.0000 | 3.34 |
| 433 | 0.3314 | 4.5044 | 13.59 |
| 434 | 0.1176 | 1.5893 | 13.52 |
| 435 | 0.2302 | 4.2477 | 18.45 |
| 436 | 0.0150 | 0.2259 | 15.10 |
| 437 | 0.0248 | 0.2921 | 11.79 |
| 438 | 0.0318 | 0.3568 | 11.23 |
| 439 | 0.0044 | 0.0438 | 10.04 |
| 440 | 0.0323 | 0.4532 | 14.01 |
| 441 | 0.0117 | 0.0995 | 8.52 |
| 442 | 0.0164 | 0.1785 | 10.89 |
| 443 | 0.0347 | 1.2679 | 36.53 |
| 444 | 0.0130 | 0.0275 | 2.12 |
| 445 | 0.0204 | 0.6581 | 32.29 |
| 446 | 0.0112 | 0.0317 | 2.83 |
| 447 | 0.0221 | 0.2220 | 10.05 |
| 448 | 0.2628 | 3.6691 | 13.96 |
| 449 | 0.0349 | 0.4910 | 14.08 |
| 450 | 0.0149 | 0.1829 | 12.27 |
| 451 | 0.0050 | 0.0080 | 1.60 |
| 452 | 0.0440 | 0.9577 | 21.77 |
| 453 | 0.0274 | 0.5954 | 21.75 |
| 454 | 0.0252 | 0.2129 | 8.46 |
| 455 | 5.0000 | 5.0000 | 1.00 |
| 456 | 0.0185 | 0.3983 | 21.49 |
| 457 | 0.0059 | 0.0483 | 8.14 |
| 458 | 0.0192 | 0.6539 | 34.03 |
| 459 | 0.1925 | 5.0000 | 25.98 |
| 460 | 0.0242 | 0.2350 | 9.71 |
| 461 | 1.5870 | 5.0000 | 3.15 |
| 462 | 1.7150 | 5.0000 | 2.92 |
| 463 | 0.0078 | 0.1190 | 15.21 |
| 464 | 0.0360 | 0.9860 | 27.40 |
| 465 | 0.1023 | 1.5606 | 15.25 |
| 466 | 0.1219 | 0.8323 | 6.83 |
| 467 | 0.0236 | 0.1020 | 4.32 |
| 468 | 0.0249 | 0.1907 | 7.67 |
| 469 | 2.3433 | 4.2602 | 1.82 |
| 470 | 0.0748 | 0.9542 | 12.76 |
| 471 | 0.1672 | 0.8843 | 5.29 |
| 472 | 0.0250 | 0.3729 | 14.93 |
| 473 | 0.0109 | 0.1470 | 13.46 |
| 474 | 0.2296 | 4.6942 | 20.44 |
| 475 | 0.2154 | 1.6789 | 7.79 |
| 476 | 0.0037 | 0.0366 | 9.82 |
| 477 | 0.0452 | 0.2935 | 6.50 |
| 478 | 0.0255 | 0.2500 | 9.80 |
| 479 | 0.0483 | 0.1893 | 3.92 |
| 480 | 0.0051 | 0.0602 | 11.83 |
| 481 | 0.0132 | 0.1132 | 8.58 |
| 482 | 0.0231 | 0.2336 | 10.09 |
| 483 | 0.0438 | 1.2250 | 27.99 |
| 484 | 0.0137 | 0.2894 | 21.16 |
| 485 | 0.0130 | 0.1886 | 14.45 |
| 486 | 1.1644 | 5.0000 | 4.29 |
| 487 | 0.0219 | 0.3435 | 15.66 |
| 488 | 0.0127 | 0.0908 | 7.17 |
| 489 | 0.0159 | 0.1812 | 11.37 |
| 490 | 0.0561 | 0.7866 | 14.02 |
| 491 | 0.0058 | 0.0628 | 10.83 |
| 492 | 0.0044 | 0.0191 | 4.38 |
| 493 | 0.1789 | 0.9210 | 5.15 |
| 494 | 0.0224 | 0.0985 | 4.39 |
| 495 | 0.0631 | 0.9812 | 15.54 |
| 496 | 0.1569 | 2.3859 | 15.21 |
| 497 | 0.0259 | 0.4115 | 15.92 |
| 498 | 0.0266 | 0.4395 | 16.52 |
| 499 | 0.2896 | 1.1935 | 4.12 |
| 500 | 0.2078 | 3.5151 | 16.91 |
| 501 | 0.0099 | 0.1352 | 13.59 |
| 502 | 0.0080 | 0.1205 | 14.99 |
| 503 | 0.0058 | 0.0737 | 12.62 |
| 504 | 0.0286 | 0.2131 | 7.45 |
| 505 | 0.5386 | 5.0000 | 9.28 |
| 506 | 0.0492 | 0.6452 | 13.12 |
| 507 | 0.3869 | 2.4281 | 6.28 |
| 508 | 0.0131 | 0.1374 | 10.53 |
| 509 | 0.0064 | 0.0269 | 4.21 |
| 510 | 0.0139 | 0.1417 | 10.20 |
| 511 | 0.3290 | 0.8349 | 2.54 |
| 512 | 0.0287 | 0.2174 | 7.58 |
| 513 | 0.0169 | 0.1640 | 9.70 |
| 514 | 0.0589 | 0.4647 | 7.89 |
| 515 | 0.2314 | 2.5980 | 11.23 |
| 516 | 0.0122 | 0.0850 | 6.98 |
| 517 | 0.4289 | 5.0000 | 11.66 |
| 518 | 4.5614 | 5.0000 | 1.10 |
| 519 | 0.0133 | 0.1443 | 10.89 |
| 520 | 0.0214 | 0.2633 | 12.29 |
| 521 | 0.0597 | 0.4238 | 7.10 |
| 522 | 0.0138 | 0.0621 | 4.48 |
| 523 | 0.0116 | 0.1806 | 15.53 |
| 524 | 0.0146 | 0.1710 | 11.68 |
| 525 | 0.0251 | 0.3731 | 14.85 |
| 526 | 0.0221 | 0.2503 | 11.34 |
| 527 | 0.0209 | 0.1770 | 8.47 |
| 528 | 0.0079 | 0.0336 | 4.24 |
| 529 | 0.1731 | 0.6713 | 3.88 |
| 530 | 0.0195 | 0.2177 | 11.19 |
| 531 | 0.1767 | 5.0000 | 28.30 |
| 532 | 0.0064 | 0.1197 | 18.84 |
| 533 | 0.0073 | 0.0914 | 12.51 |
| 534 | 0.0061 | 0.0763 | 12.41 |
| 535 | 0.6374 | 5.0000 | 7.84 |
| 536 | 0.0104 | 0.5899 | 56.64 |
| 537 | 0.0099 | 0.0616 | 6.24 |
| 538 | 0.1141 | 1.6741 | 14.67 |
| 539 | 0.0122 | 0.1532 | 12.53 |
| 540 | 0.0307 | 0.6325 | 20.63 |
| 541 | 0.1312 | 5.0000 | 38.11 |
| 542 | 0.0168 | 0.2288 | 13.60 |
| 543 | 0.0202 | 0.3829 | 18.97 |
| 544 | 0.0418 | 1.0209 | 24.42 |
| 545 | 0.0077 | 0.0659 | 8.60 |
| 546 | 1.8162 | 5.0000 | 2.75 |
| 547 | 0.2928 | 5.0000 | 17.08 |
| 548 | 0.0054 | 0.0517 | 9.59 |
| 549 | 0.0073 | 0.1308 | 17.80 |
| 550 | 0.0053 | 0.0639 | 12.09 |
| 551 | 0.0203 | 0.3986 | 19.65 |
| 552 | 0.0098 | 0.1903 | 19.48 |
| 553 | 0.0204 | 0.4576 | 22.48 |
| 554 | 0.0086 | 0.0831 | 9.64 |
| 555 | 0.0046 | 0.0250 | 5.44 |
| 556 | 1.6996 | 5.0000 | 2.94 |
| 557 | 0.0113 | 0.2354 | 20.89 |
| 558 | 0.0093 | 0.1387 | 14.96 |
| 559 | 3.9323 | 5.0000 | 1.27 |
| 560 | 0.4980 | 5.0000 | 10.04 |
| 561 | 0.0672 | 0.8394 | 12.50 |
| 562 | 0.4248 | 5.0000 | 11.77 |
| 563 | 0.0271 | 0.4485 | 16.52 |
| 564 | 0.0336 | 0.8813 | 26.25 |
| 565 | 0.0115 | 0.1383 | 12.06 |
| 566 | 0.0326 | 0.6786 | 20.83 |
| 567 | 0.0328 | 0.5340 | 16.28 |
| 568 | 0.1063 | 1.0575 | 9.95 |
| 569 | 0.1557 | 0.5172 | 3.32 |
| 570 | 0.0669 | 0.5570 | 8.32 |
| 571 | 0.3314 | 2.2781 | 6.87 |
| 572 | 0.0084 | 0.1356 | 16.08 |
| 573 | 0.0079 | 0.0438 | 5.56 |
| 574 | 0.2738 | 1.7766 | 6.49 |
| 575 | 0.0054 | 0.0823 | 15.34 |
| 576 | 0.0889 | 1.6763 | 18.86 |
| 577 | 0.5693 | 2.1548 | 3.78 |
| 578 | 0.0277 | 0.5255 | 19.00 |
| 579 | 0.0084 | 0.0369 | 4.40 |
| 580 | 0.1058 | 1.7642 | 16.67 |
| 581 | 0.1554 | 2.3662 | 15.23 |
| 582 | 1.2358 | 5.0000 | 4.05 |
| 583 | 0.1087 | 1.8929 | 17.41 |
| 584 | 0.7925 | 5.0000 | 6.31 |
| 585 | 0.0401 | 0.3693 | 9.22 |
| 586 | 0.0055 | 0.0819 | 14.77 |
| 587 | 0.0112 | 0.0639 | 5.69 |
| 588 | 0.0289 | 0.4117 | 14.25 |
| 589 | 0.0777 | 1.0786 | 13.88 |
| 590 | 0.0224 | 0.1774 | 7.93 |
| 591 | 0.0220 | 0.2420 | 10.98 |
| 592 | 0.0529 | 1.6780 | 31.69 |
| 593 | 0.2429 | 2.1903 | 9.02 |
| 594 | 0.0372 | 1.1862 | 31.92 |
| 595 | 0.4078 | 3.5038 | 8.59 |
| 596 | 0.6812 | 5.0000 | 7.34 |
| 597 | 0.0413 | 0.4795 | 11.61 |
| 598 | 0.0293 | 0.9907 | 33.79 |
| 599 | 0.0588 | 0.7367 | 12.54 |
| 600 | 0.0132 | 0.1760 | 13.32 |
| 601 | 0.2150 | 1.9096 | 8.88 |
| 602 | 0.0158 | 0.1579 | 9.98 |
| 603 | 0.0107 | 0.1169 | 10.88 |
| 604 | 0.0057 | 0.0569 | 10.00 |
| 605 | 0.0117 | 0.1869 | 16.04 |
| 606 | 0.0075 | 0.1246 | 16.72 |
| 607 | 0.0446 | 0.7023 | 15.74 |
| 608 | 0.0445 | 0.9524 | 21.42 |
| 609 | 0.0392 | 1.1137 | 28.40 |
| 610 | 0.0513 | 1.1099 | 21.62 |
| 611 | 0.0388 | 0.7132 | 18.38 |
| 612 | 0.0266 | 0.3497 | 13.16 |
| 613 | 0.2134 | 2.2212 | 10.41 |
| 614 | 0.0951 | 0.8615 | 9.06 |
| 615 | 0.0221 | 0.3581 | 16.22 |
| 616 | 0.0082 | 0.4424 | 54.03 |
| 617 | 0.0904 | 2.5430 | 28.12 |
| 618 | 0.0090 | 0.1260 | 14.00 |
| 619 | 0.0056 | 0.0627 | 11.17 |
| 620 | 0.0168 | 0.1577 | 9.37 |
| 621 | 0.0749 | 0.4080 | 5.44 |
| 622 | 0.0274 | 0.1092 | 3.98 |
| 623 | 0.0110 | 0.0236 | 2.15 |
| 624 | 0.0200 | 0.0381 | 1.90 |
| 625 | 0.0367 | 0.5150 | 14.05 |
| 626 | 0.0200 | 0.4614 | 23.08 |
| 627 | 0.0039 | 0.0050 | 1.28 |
| 628 | 0.0027 | 0.0097 | 3.60 |
| 629 | 0.0100 | 0.0850 | 8.49 |
| 630 | 0.0480 | 1.1578 | 24.14 |
| 631 | 1.1941 | 5.0000 | 4.19 |
| 632 | 0.0583 | 1.1295 | 19.38 |
| 633 | 2.2349 | 5.0000 | 2.24 |
| 634 | 0.0073 | 0.0819 | 11.28 |
| 635 | 0.1677 | 2.5286 | 15.08 |
| 636 | 0.2709 | 4.2631 | 15.73 |
| 637 | 0.0200 | 0.2074 | 10.34 |
| 638 | 0.2266 | 0.8829 | 3.90 |
| 639 | 0.1017 | 0.7795 | 7.66 |
| 640 | 0.6151 | 4.4017 | 7.16 |
| 641 | 0.0136 | 0.1574 | 11.56 |
| 642 | 0.1562 | 2.9466 | 18.87 |
| 643 | 0.1082 | 0.8887 | 8.21 |
| 644 | 0.0064 | 0.1696 | 26.34 |
| 645 | 0.0098 | 0.1705 | 17.38 |
| 646 | 0.0121 | 0.2472 | 20.44 |
| 647 | 0.0559 | 0.3697 | 6.61 |
| 648 | 0.0050 | 0.0194 | 3.85 |
| 649 | 0.1265 | 0.7502 | 5.93 |
| 650 | 0.0127 | 0.1147 | 9.04 |
| 651 | 0.0553 | 1.7007 | 30.77 |
| 652 | 0.4428 | 3.1913 | 7.21 |
| 653 | 0.2303 | 1.7209 | 7.47 |
| 654 | 0.1037 | 2.5701 | 24.78 |
| 655 | 0.2489 | 3.9320 | 15.80 |
| 656 | 0.0122 | 0.1281 | 10.50 |
| 657 | 0.0353 | 0.2019 | 5.73 |
| 658 | 0.0070 | 0.3418 | 48.50 |
| 659 | 0.0427 | 1.0364 | 24.28 |
| 660 | 0.0072 | 0.0590 | 8.17 |
| 661 | 0.0439 | 0.3011 | 6.86 |
| 662 | 0.0367 | 0.1726 | 4.71 |
| 663 | 0.6730 | 5.0000 | 7.43 |
| 664 | 1.7471 | 5.0000 | 2.86 |
| 665 | 0.0064 | 0.0868 | 13.54 |
| 666 | 0.0242 | 0.2677 | 11.06 |
| 667 | 0.0390 | 0.5165 | 13.24 |
| 668 | 0.0084 | 0.0734 | 8.70 |
| 669 | 0.0171 | 0.3236 | 18.96 |
| 670 | 0.0101 | 0.0883 | 8.79 |
| 671 | 4.0045 | 5.0000 | 1.25 |
| 672 | 0.0152 | 0.2086 | 13.70 |
| 673 | 0.5679 | 4.9915 | 8.79 |
| 674 | 3.7806 | 5.0000 | 1.32 |
| 675 | 0.8839 | 5.0000 | 5.66 |
| 676 | 0.0030 | 0.0355 | 11.70 |
| 677 | 0.0038 | 0.0357 | 9.28 |
| 678 | 0.0545 | 0.5487 | 10.07 |
| 679 | 0.0189 | 0.4228 | 22.41 |
| 680 | 0.0327 | 0.9694 | 29.69 |
| 681 | 0.0330 | 0.3147 | 9.53 |
| 682 | 0.0103 | 0.1643 | 15.88 |
| 683 | 0.0763 | 0.7954 | 10.42 |
| 684 | 0.4840 | 5.0000 | 10.33 |
| 685 | 0.7152 | 5.0000 | 6.99 |
| 686 | 0.0143 | 0.1994 | 13.91 |
| 687 | 0.0197 | 0.3603 | 18.34 |
| 688 | 0.2574 | 1.7119 | 6.65 |
| 689 | 0.0402 | 0.8817 | 21.94 |
| 690 | 0.0235 | 1.2862 | 54.81 |
| 691 | 0.0095 | 0.1589 | 16.64 |
| 692 | 0.0060 | 0.0849 | 14.09 |
| 693 | | | |
| 694 | 0.0342 | 0.2868 | 8.38 |
| 695 | 0.0433 | 0.4204 | 9.72 |
| 696 | 0.0068 | 0.1284 | 18.76 |
| 697 | 0.0049 | 0.0379 | 7.66 |
| 698 | 0.1930 | 4.7234 | 24.47 |
| 699 | 0.2751 | 2.2127 | 8.04 |
| 700 | 0.0145 | 0.7576 | 52.11 |
| 701 | 0.0872 | 0.8685 | 9.96 |
| 702 | 0.0102 | 0.9599 | 93.83 |
| 703 | 0.0368 | 0.2982 | 8.10 |
| 704 | 0.0911 | 2.1107 | 23.18 |
| 705 | 0.0043 | 0.0581 | 13.63 |
| 706 | 0.0050 | 0.0469 | 9.39 |
| 707 | 0.0546 | 0.2641 | 4.84 |
| 708 | 0.0529 | 0.9073 | 17.15 |
| 709 | 0.0376 | 0.4355 | 11.57 |
| 710 | 0.0298 | 0.1526 | 5.13 |
| 711 | 0.0093 | 0.2548 | 27.53 |
| 712 | 0.0086 | 0.1024 | 11.90 |
| 713 | 0.0459 | 0.9865 | 21.51 |
| 714 | 0.0549 | 0.6674 | 12.16 |
| 715 | 0.0054 | 0.0221 | 4.12 |
| 716 | 1.7325 | 4.7719 | 2.75 |
| 717 | 0.0770 | 0.4322 | 5.61 |
| 718 | 0.0105 | 0.0996 | 9.44 |
| 719 | 0.0426 | 0.5801 | 13.62 |
| 720 | 0.0436 | 0.2312 | 5.30 |
| 721 | 0.1879 | 5.0000 | 26.60 |
| 722 | 0.0088 | 0.7163 | 81.47 |
| 723 | 0.0402 | 0.6673 | 16.58 |
| 724 | 0.1222 | 2.2493 | 18.41 |
| 725 | 0.0268 | 0.2937 | 10.95 |
| 726 | 0.0247 | 0.2925 | 11.83 |
| 727 | 0.1033 | 1.2228 | 11.84 |
| 728 | 0.0123 | 0.0296 | 2.42 |
| 729 | 0.0446 | 1.9188 | 42.98 |
| 730 | 0.0164 | 0.8136 | 49.65 |
| 731 | 0.0184 | 0.1984 | 10.80 |
| 732 | 0.0093 | 0.0925 | 9.97 |
| 733 | 0.0094 | 0.0842 | 8.96 |
| 734 | 0.0063 | 0.0862 | 13.67 |
| 735 | 0.0082 | 0.1101 | 13.49 |
| 736 | 1.1376 | 3.8956 | 3.42 |
| 737 | 0.0104 | 0.2270 | 21.91 |
| 738 | 0.0132 | 0.4281 | 32.33 |
| 739 | 0.0127 | 0.2213 | 17.37 |
| 740 | 0.0482 | 1.0808 | 22.41 |
| 741 | 0.0066 | 0.0934 | 14.12 |
| 742 | 0.0240 | 0.4781 | 19.92 |
| 743 | 0.0275 | 0.1196 | 4.35 |
| 744 | 0.4317 | 3.9395 | 9.13 |
| 745 | 0.0045 | 0.0279 | 6.27 |
| 746 | 0.0222 | 0.5671 | 25.51 |
| 747 | 0.0088 | 0.1184 | 13.42 |
| 748 | 0.0121 | 0.1537 | 12.75 |
| 749 | 0.0126 | 0.2095 | 16.64 |
| 750 | 0.0040 | 0.0183 | 4.62 |
| 751 | 0.0362 | 2.2969 | 63.48 |
| 752 | 1.0370 | 3.8127 | 3.68 |
| 753 | 0.0142 | 0.2025 | 14.28 |
| 754 | 0.0191 | 0.1915 | 10.04 |
| 755 | 0.0071 | 0.1244 | 17.47 |
| 756 | 0.0151 | 0.1761 | 11.62 |
| 757 | 0.0149 | 0.2944 | 19.81 |
| 758 | 0.0356 | 0.5066 | 14.23 |
| 759 | 0.0053 | 0.0533 | 9.97 |
| 760 | 0.0088 | 0.0622 | 7.05 |
| 761 | 0.0087 | 0.0679 | 7.77 |
| 762 | 5.0000 | 5.0000 | 1.00 |
| 763 | 0.0053 | 0.0454 | 8.52 |
| 764 | 0.0095 | 0.1588 | 16.66 |
| 765 | 0.0235 | 0.2844 | 12.08 |
| 766 | 0.0171 | 0.1791 | 10.47 |
| 767 | 0.0174 | 0.1941 | 11.17 |
| 768 | 0.0107 | 0.1257 | 11.76 |
| 769 | 0.0084 | 0.0264 | 3.16 |
| 770 | 0.0164 | 0.3387 | 20.67 |
| 771 | 0.0105 | 0.1718 | 16.35 |
| 772 | 0.3971 | 2.8809 | 7.26 |
| 773 | 0.0385 | 1.1656 | 30.25 |
| 774 | 0.0120 | 0.1737 | 14.44 |
| 775 | 0.0125 | 0.1519 | 12.13 |
| 776 | 0.0063 | 0.1001 | 15.77 |
| 777 | 0.0740 | 0.9814 | 13.26 |
| 778 | 0.0258 | 0.4153 | 16.09 |
| 779 | 0.0055 | 0.0117 | 2.13 |
| 780 | 0.0290 | 0.5153 | 17.78 |
| 781 | 0.0279 | 0.7132 | 25.53 |
| 782 | 0.0087 | 0.0435 | 4.99 |
| 783 | 0.0311 | 0.4443 | 14.27 |
| 784 | 0.0246 | 0.2123 | 8.62 |
| 785 | 0.0858 | 1.3287 | 15.49 |
| 786 | 0.0156 | 0.1583 | 10.14 |
| 787 | 0.0135 | 0.1937 | 14.38 |
| 788 | 0.6197 | 4.1467 | 6.69 |
| 789 | 0.0059 | 0.0303 | 5.10 |
| 790 | 0.0122 | 0.0614 | 5.03 |
| 791 | 0.0197 | 0.0435 | 2.21 |
| 792 | 0.0137 | 0.1352 | 9.88 |
| 793 | 0.0118 | 0.1403 | 11.88 |
| 794 | 0.0066 | 0.0304 | 4.60 |
| 795 | 0.1222 | 4.1141 | 33.67 |
| 796 | 0.0208 | 0.2723 | 13.06 |
| 797 | 0.0188 | 0.1897 | 10.11 |
| 798 | 0.0074 | 0.0438 | 5.90 |
| 799 | 0.0255 | 0.6629 | 26.04 |
| 800 | 0.1686 | 0.6359 | 3.77 |
| 801 | 0.1110 | 1.4430 | 13.00 |
| 802 | 0.0081 | 0.0671 | 8.31 |
| 803 | 0.0081 | 0.1048 | 12.88 |
| 804 | 0.0158 | 0.6462 | 40.93 |
| 805 | 0.0262 | 0.3025 | 11.56 |
| 806 | 0.0923 | 0.8006 | 8.67 |
| 807 | 0.0191 | 0.4860 | 25.47 |
| 808 | 0.0840 | 1.0502 | 12.50 |
| 809 | 0.0069 | 0.2694 | 39.28 |
| 810 | 0.0083 | 0.1854 | 22.29 |
| 811 | 5.0000 | 5.0000 | 1.00 |
| 812 | 0.0458 | 0.6618 | 14.46 |
| 813 | 0.0381 | 0.4772 | 12.52 |
| 814 | 0.0214 | 0.2223 | 10.40 |
| 815 | 0.1927 | 1.4460 | 7.51 |
| 816 | 0.0423 | 0.7638 | 18.04 |
| 817 | 0.0124 | 0.1431 | 11.55 |
| 818 | 0.0624 | 1.6796 | 26.90 |
| 819 | 0.0064 | 0.0467 | 7.26 |
| 820 | 0.0961 | 1.3756 | 14.31 |
| 821 | 0.0797 | 0.9590 | 12.03 |
| 822 | 0.0367 | 0.4576 | 12.47 |
| 823 | 0.0613 | 1.0931 | 17.84 |
| 824 | 0.7420 | 4.8192 | 6.49 |
| 825 | 0.0319 | 0.2456 | 7.70 |
| 826 | 3.1971 | 5.0000 | 1.56 |
| 827 | 0.0131 | 0.1526 | 11.63 |
| 828 | 0.0451 | 2.3687 | 52.48 |
| 829 | 1.6557 | 5.0000 | 3.02 |
| 830 | 0.5764 | 2.7603 | 4.79 |
| 831 | 0.0352 | 1.1635 | 33.09 |
| 832 | 0.0155 | 0.9056 | 58.26 |
| 833 | 2.3216 | 5.0000 | 2.15 |
| 834 | 0.0202 | 0.2909 | 14.40 |
| 835 | 0.0171 | 0.1561 | 9.15 |
| 836 | 0.1048 | 0.6505 | 6.21 |
| 837 | 0.0330 | 0.1634 | 4.95 |
| 838 | 0.0275 | 1.0648 | 38.76 |
| 839 | 5.0000 | 5.0000 | 1.00 |
| 840 | 3.9984 | 5.0000 | 1.25 |
| 841 | 0.2902 | 2.0763 | 7.16 |
| 842 | 0.0299 | 0.3288 | 10.98 |
| 843 | 0.0562 | 0.4837 | 8.61 |
| 844 | 0.0190 | 0.3795 | 19.99 |
| 845 | 0.0212 | 0.1838 | 8.66 |
| 846 | 0.4062 | 4.5733 | 11.26 |
| 847 | 0.0194 | 0.3762 | 19.41 |
| 848 | 0.2156 | 2.1791 | 10.10 |
| 849 | 0.6060 | 5.0000 | 8.25 |
| 850 | 0.6637 | 4.5607 | 6.87 |
| 851 | 0.7080 | 2.5737 | 3.64 |
| 852 | 0.0098 | 0.1764 | 17.95 |
| 853 | 0.0126 | 0.2050 | 16.24 |
| 854 | 0.1049 | 2.0699 | 19.74 |
| 855 | 0.3953 | 3.7956 | 9.60 |
| 856 | 0.0119 | 0.6638 | 55.91 |

### Example 6. Synthesis of Compound A

BRG1/BRM Inhibitor compound A has the structure:

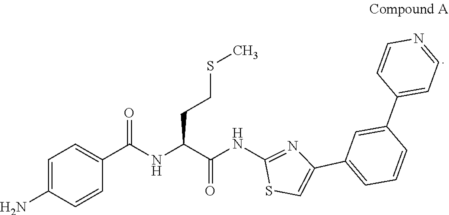 [SMILES: CSCC[C@H](NC(=O)c1ccc(N)cc1)C(=O)Nc1nc(-c2cccc(-c3ccncc3)c2)cs1]

Compound A was synthesized as shown in Scheme 1 below.

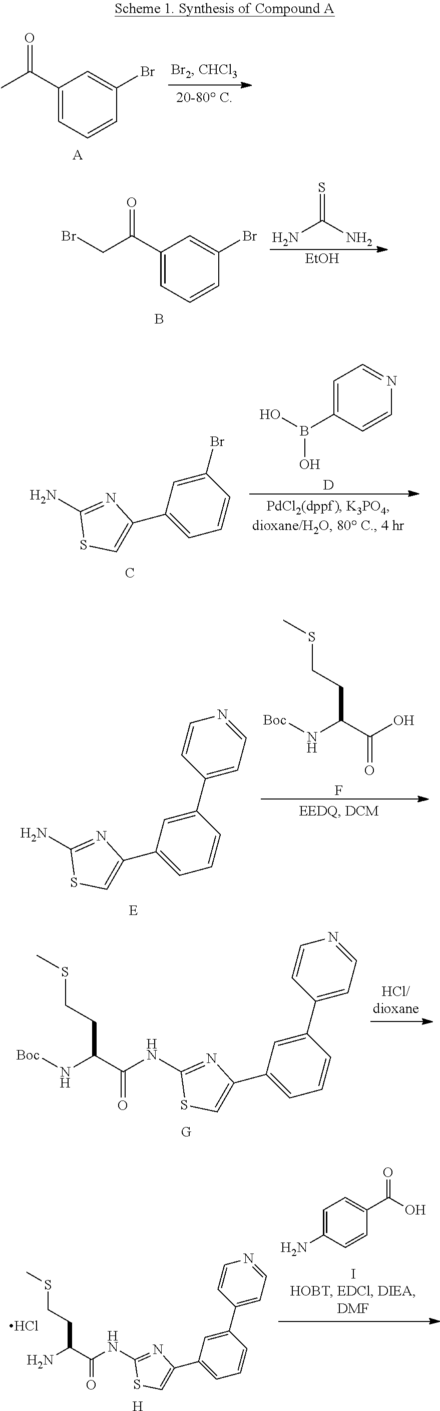

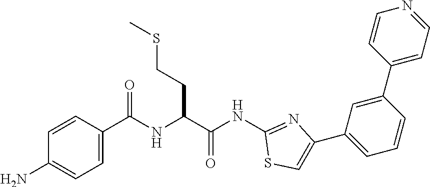 [SMILES: CSCC[C@H](NC(=O)c1ccc(N)cc1)C(=O)Nc1nc(-c2cccc(-c3ccncc3)c2)cs1]

The ATPase catalytic activity of BRM or BRG-1 in the presence of Compound A was measured by the in vitro biochemical assay using ADP-Glo™ (Promega, V9102) described above. Compound A was found to have an IC₅₀ of 10.4 nM against BRM and 19.3 nM against BRG1 in the assay.

### Example 7. Effects of BRG1/BRM ATPase Inhibition on the Growth of Uveal Melanoma and Hematological Cancer Cell Lines

Procedure: Uveal melanoma cell lines (92-1, MP41, MP38, MP48), prostate cancer cell lines (LNCAP), lung cancer cell lines (NCI-H1299), and immortalized embryonic kidney lines (HEK293T) were plated into 96 well plates with growth media (see Table 6). BRG1/BRM ATPase inhibitor, Compound A, was dissolved in DMSO and added to the cells in a concentration gradient from 0 to 10 micromolar at the time of plating. Cells were incubated at 37 degrees Celsius for 3 days. After three days of treatment, the media was removed from the cells and 30 microliters of TrypLE (Gibco) was added to cells for 10 minutes. Cells were detached from the plates and resuspended with the addition of 170 microliters of growth media. Cells from two DMSO-treated control wells were counted, and the initial number of cells plated at the start of the experiment, were re-plated into fresh-compound containing plates for an additional four days at 37 degrees Celsius. At day 7, cells were harvested as described above. On day 3 and day 7, relative cell growth was measured by the addition of Cell-titer glo (Promega) and luminescence was measured on an Envision plate reader (Perkin Elmer). The concentration of compound at which each cell line's growth was inhibited by 50% (GI₅₀), was calculated using Graphpad Prism, and is plotted below. For multiple myeloma cell lines (OPM2, MM1S, LP1), ALL cell lines (TALL1, JURKAT, RS411), DLBCL cell lines (SUDHL6, SUDHL4, DB, WSUDLCL2, PFEIFFER), AML cell lines (OCIAML5), MDS cell lines (SKM1), ovarian cancer cell lines (OW, TYKNU), esophageal cancer cell lines (KYSE150), rhabdoid tumor lines (RD, G402, G401, HS729, A204), liver cancer cell lines (HLF, HLE, PLCRPF5), and lung cancer cell lines (SW1573, NCIH2444), the above methods were performed with the following modifications: Cells were plated in 96 well plates, and the next day, BRG1/BRM ATPase inhibitor, Compound A, was dissolved in DMSO and added to the cells in a concentration gradient from 0 to 10 micromolar. At the time of cell splitting on days 3 and 7, cells were split into new 96 well plates, and fresh compound was added four hours after re-plating.

Table 6 lists the tested cell lines and growth media used.

TABLE 6
Cell Lines and Growth Media

| Cell Line | Source | Growth Media |
|---|---|---|
| 92-1 | SIGMA | RPMI1640 + 20% FBS |
| A204 | ATCC | McCoy's 5A + 10% FBS |
| DB | ATCC | RPMI1640 + 10% FBS |
| G401 | ATCC | McCoy's 5A + 10% FBS |
| G402 | ATCC | McCoy's 5A + 10% FBS |
| HEK293T | ATCC | DMEM + 10% FBS |
| HLE | CRB | DMEM + 10% FBS |
| HLF | JCRB | DMEM + 10% FBS |
| HS729 | ATCC | DMEM + 10% FBS |
| JURKAT | ATCC | RPMI1640 + 10% FBS |
| KYSE150 | DSMZ | RPMI1640/Ham's F12 + 10% FBS |
| LNCAP | ATCC | RPMI1640 + 10% FBS |
| LP1 | DSMZ | IMDM + 20% FBS |
| MM1S | ATCC | RPMI1640 + 10% FBS |
| MP38 | ATCC | RPMI1640 + 20% FBS |
| MP41 | ATCC | RPMI1640 + 20% FBS |
| MP46 | ATCC | RPMI1640 + 20% FBS |
| NCIH1299 | ATCC | RPMI1640 + 10% FBS |
| NCIH2444 | ATCC | RPMI1640 + 20% FBS |
| OCIAML5 | DSMZ | alpha-MEM + 20% FBS + 10 ng/ml GM-CSF |
| OPM2 | DSMZ | RPMI1640 + 10% FBS |
| OV7 | ECACC | DMEM/Ham's F12 (1:1) + 2 mM Glutamine + 10% |
| | | FBS + 0.5 ug/ml hydrocortisone + 10 ug/ml insulin |
| PFEIFFER | ATCC | RPMI1640 + 10% FBS |
| PLCPRF5 | ATCC | EMEM + 10% FBS |
| RD | ATCC | DMEM + 10% FBS |
| RS411 | ATCC | RPMI1640 + 10% FBS |
| SKM1 | JCRB | RPMI1640 + 10% FBS |
| SUDHL4 | DSMZ | RPMI1640 + 10% FBS |
| SUDHL6 | ATCC | RPMI1640 + 20% FBS |
| SW1573 | ATCC | DMEM + 10% FBS |
| TALL1 | ICRB | RPMI1640 + 10% FBS |
| TYKNU | JCRB | EMEM + 20% FBS |
| WSUDLCL2 | DSMZ | RPMI1640 + 10% FBS |

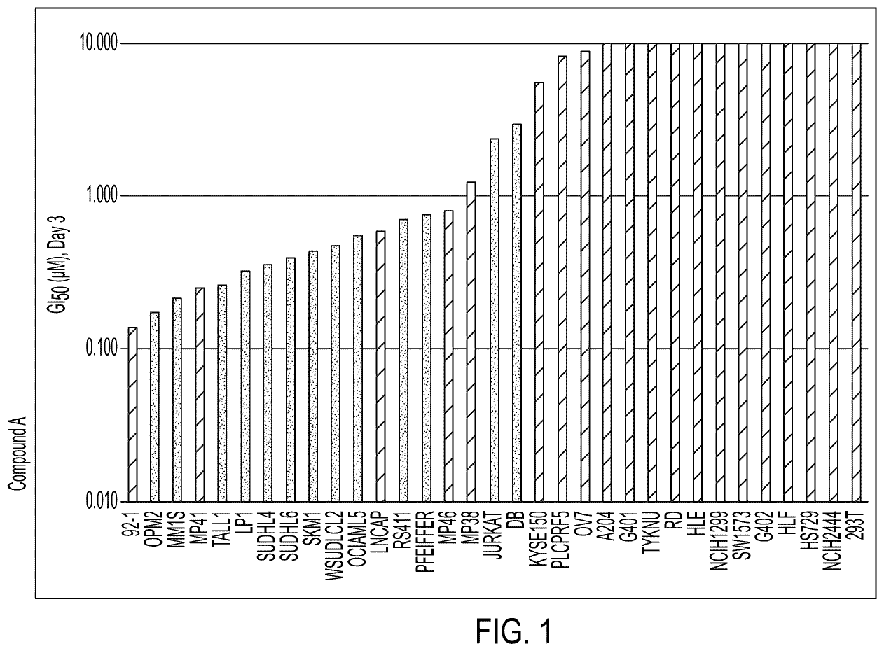

Results: As shown in FIG. 1, the uveal melanoma and hematologic cancer cell lines were more sensitive to BRG1/BRM inhibition than the other tested cell lines. Inhibition of the uveal melanoma and hematologic cancer cell lines was maintained through day 7.

### Example 8. Comparison of BRG1/BRM Inhibitors to Clinical PKC and MEK Inhibitors in Uveal Melanoma Cell Lines

Procedure: Uveal melanoma cell lines, 92-1 or MP41, were plated in 96 well plates in the presence of growth media (see Table 5). BAF ATPase inhibitors (Compound A), PKC inhibitor (LXS196; MedChemExpress), or MEK inhibitor (Selumetinib; Selleck Chemicals) were dissolved in DMSO and added to the cells in a concentration gradient from 0 to 10 micromolar at the time of plating. Cells were incubated at 37 degrees Celsius for 3 days. After three days of treatment, cell growth was measured with Cell-titer glow (Promega), and luminescence was read on an Envision plate reader (Perkin Elmer).

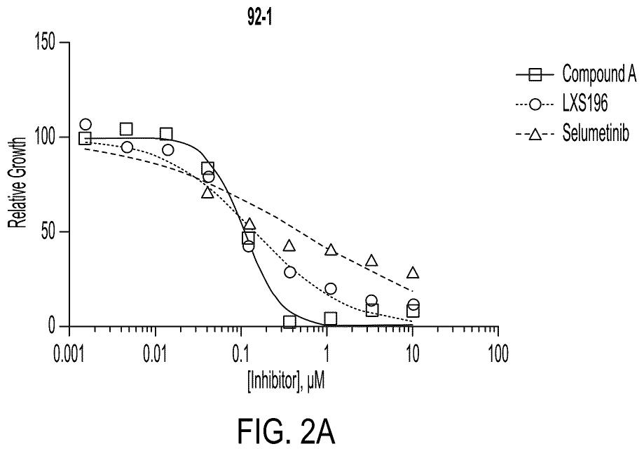

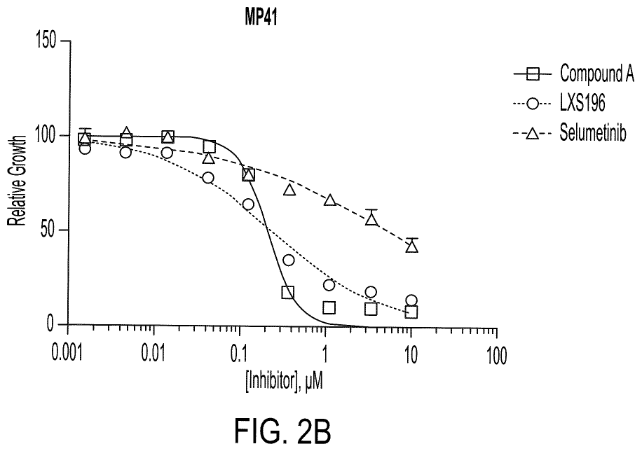

Results: As shown in FIG. 2A and FIG. 2B, Compound A showed comparable growth inhibition of uveal melanoma cells as the clinical PKC and MEK inhibitors. Further, compound A was found to result in a faster onset of inhibition than the clinical PKC and MEK inhibitors.

### Example 9. Synthesis of Compound B

BRG1/BRM Inhibitor Compound B has the structure:

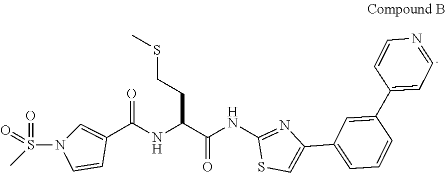 [SMILES: CSCC[C@H](NC(=O)c1ccn(S(C)(=O)=O)c1)C(=O)Nc1nc(-c2cccc(-c3ccncc3)c2)cs1]

Compound B was synthesized as shown in Scheme 2 below.

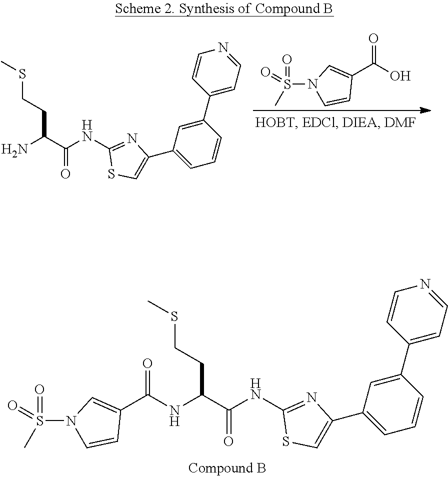

### Preparation of (S)-1-(methylsulfonyl)-N-(4-(methylthio)-1-oxo-1-((4-(3-(pyridin-4-yl)phenyl)thiazol-2-yl)amino)butan-2-yl)-1H-pyrrole-3-carboxamide (Compound B)

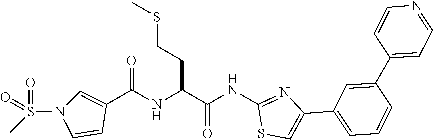 [SMILES: CSCC[C@H](NC(=O)c1ccn(S(C)(=O)=O)c1)C(=O)Nc1nc(-c2cccc(-c3ccncc3)c2)cs1]

To a mixture of (2S)-2-amino-4-methylsulfanyl-N-[4-[3-(4-pyridyl)phenyl]thiazol-2-yl]butanamide (2 g, 4.75 mmol, HCl salt) and 1-methylsulfonylpyrrole-3-carboxylic acid (898.81 mg, 4.75 mmol) in DMF (20 mL) was added EDCl (1.37 g, 7.13 mmol), HOBt (982.92 mg, 7.13 mmol), and DIEA (2.46 g, 19.00 mmol, 3.31 mL) and the mixture was stirred at 25° C. for 3 hours. The mixture was poured into H₂O (100 mL) and the precipitate was collected by filtration. The solid was triturated in MeOH (20 mL) and the precipitate was collected by filtration. The solid was dissolved in DMSO (10 mL) and then the mixture was poured into MeOH (50 mL) and the formed precipitate was collected by filtration and lyophilized to give Compound B (2.05 g, 3.88 mmol, 77.01% yield) as a white solid. LCMS (ESI) m/z [M+H]⁺=555.9. ¹H NMR (400 MHz, DMSO) δ 12.49 (s, 1H), 8.68-8.66 (m, 2H), 8.46 (d, J=7.2 Hz, 1H), 8.31-8.30 (m, 1H), 8.02-8.00 (m, 1H), 7.94-7.96 (m, 1H), 7.83 (s, 1H), 7.73-7.74 (m, 3H), 7.61-7.57 (m, 1H), 7.31-7.29 (m, 1H), 8.79-6.77 (m, 1H), 4.74-4.89 (m, 1H), 3.57 (s, 3H), 2.67-2.53 (m, 2H), 2.13-2.01 (m, 5H). SFC: AS-3-MeOH (DEA)-40-3 mL-35T.Icm, t=0.932 min, ee %=100%.

### Example 10. Effects of BRG1/BRM ATPase Inhibition on the Growth of Weal Melanoma, Hematological Cancer, Prostate Cancer, Breast Cancer, and Ewing's Sarcoma Cell Lines

Procedure: All cell lines described above in Example 7 were also tested as described above with Compound B. In addition, the following cell lines were also tested as follows. Briefly, for Ewing's sarcoma cell lines (CADOES1, RDES, SKES1), retinoblastoma cell lines (WERIRB1), ALL cell lines (REH), AML cell lines (KASUMI1), prostate cancer cell lines (PC3, DU145, 22RV1), melanoma cell lines (SH4, SKMEL28, WM115, COL0829, SKMEL3, A375), breast cancer cell lines (MDAMB415, CAMA1, MCF7, BT474, HCC1419, DU4475, BT549), B-ALL cell lines (SUPB15), CML cell lines (K562, MEG01), Burkitt's lymphoma cell lines (RAMOS2G64C10, DAUDI), mantle cell lymphoma cell lines (JEKO1, REC1), bladder cancer cell lines (HT1197), and lung cancer cell lines (SBC5), the above methods were performed with the following modifications: Cells were plated in 96 well plates, and the next day, BRG1/BRM ATPase inhibitor, Compound B, was dissolved in DMSO and added to the cells in a concentration gradient from 0 to 10 micromolar. At the time of cell splitting on days 3 and 7, cells were split into new 96 well plates, and fresh compound was added four hours after re-plating.

Table 7 lists the tested cell lines and growth media used.

TABLE 7
Cell Lines And Growth Media

| Call Line | Source | Growth Media |
|---|---|---|
| 22RV1 | ATCC | RPMI1640 + 10% FBS |
| A375 | ATCC | DMEM + 10% FBS |
| BT474 | ATCC | Hybricare medium + 1.5 g/L sodium bicarbonate + 10% FBS |
| BT549 | ATCC | RPMI1640 + 0.023 IU/ml insulin + 10% FBS |
| CADOES1 | DSMZ | RPMI1640 + 10% FBS |
| CAMA1 | ATCC | EMEM + 10% FBS |
| COLO829 | ATCC | RPMI1640 + 10% FBS |
| DAUDI | ATCC | RPMI1640 + 10% FBS |
| DU145 | ATCC | EMEM + 10% FBS |
| DU4475 | ATCC | RPMI1540 + 10% FBS |
| HCC1419 | ATCC | RPMI1640 + 10% FBS |
| HT1197 | ATCC | EMEM + 10% FBS |
| JEKO1 | ATCC | RPMI 1640 + 20% FBS |
| K5 62 | ATCC | IMDM + 10% FBS |
| KASUMI1 | ATCC | RPMI1640 + 10% FBS |
| MCF7 | ATCC | EMEM + 0.01 mg/ml bovine insulin + 10% FBS |
| MDAMB415 | ATCC | Leibovitz's L-15 + 2 mM L-glutamine + 10 mcg/ml |
| | | insulin + 10 mcg/ml glutathione + 15% FBS |
| MEG01 | ATCC | RPMI1640 + 10% FBS |
| PC3 | ATCC | F-12K + 10% FBS |
| RAMOS2G64C10 | ATCC | RPMI1640 + 10% FBS |
| RDES | ATCC | RPMI1640 + 15% FBS |
| REC1 | ATCC | RPMI1640 + 10% FBS |
| REH | ATCC | RPMI1640 + 10% FBS |
| SBC5 | JCRB | EMEM + 10% FBS |
| SH4 | ATCC | DMEM + 10% FBS |
| ISKES1 | ATCC | McCoy's 5A + 15% FBS |
| ISKMEL28 | ATCC | EMEM + 10% FBS |
| SKMEL3 | ATCC | McCoy's 5A + 15% FBS |
| SUPB15 | ATCC | IMDM + 4 mM L-glutamine + 1.5 g/L sodium |
| | | bicarbonate + 0.05 mM 2-mercaptoethanol + 20% FBS |
| WERIRB1 | ATCC | RPMI1640 + 10% FBS |
| WM115 | ATCC | EMEM + 10% FBS |

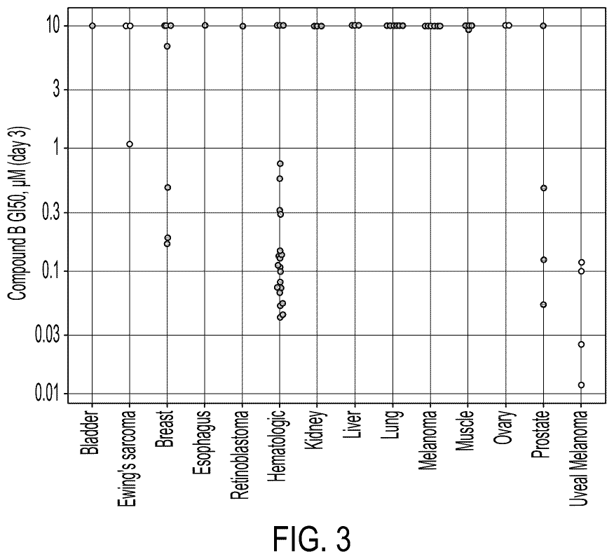

Results: As shown in FIG. 3, the uveal melanoma, hematologic cancer, prostate cancer, breast cancer, and Ewing's sarcoma cell lines were more sensitive to BRG1/BRM Inhibition than the other tested cell lines. Inhibition of the uveal melanoma, hematologic cancer, prostate cancer, breast cancer, and Ewing's sarcoma cell lines was maintained through day 7.

### Example 11. Effects of BRG1/BRM ATPase inhibition on the growth of cancer cell lines

Procedure: A pooled cell viability assay was performed using PRISM (Profiling Relative Inhibition Simultaneously in Mixtures) as previously described (“High-throughput identification of genotype-specific cancer vulnerabilities in mixtures of barcoded tumor cell lines”, Yu et al, Nature Biotechnology 34, 419-423, 2016), with the following modifications. Cell lines were obtained from the Cancer Cell Line Encyclopedia (CCLE) collection and adapted to RPMI-1640 medium without phenol red, supplemented with 10% heat-inactivated fetal bovine serum (FBS), in order to apply a unique infection and pooling protocol to such a big compendium of cell lines. A lentiviral spin-infection protocol was executed to introduce a 24 nucleotide-barcode in each cell line, with an estimated multiplicity of infection (MOI) of 1 for all cell lines, using blasticidin as selection marker. Over 750 PRISM cancer cell lines stably barcoded were then pooled together according to doubling time in pools of 25. For the screen execution, instead of plating a pool of 25 cell lines in each well as previously described (Yu et al.), all the adherent or all the suspension cell line pools were plated together using T25 flasks (100,000 cells/flask) or 6-well plates (50,000 cells/well), respectively. Cells were treated with either DMSO or compound in a 8-point 3 fold dose response in triplicate, starting from a top concentration of 10 μM. As control for assay robustness, cells were treated in parallel with two previously validated compounds, the pan-Raf inhibitor AZ-628, and the proteasome inhibitor bortezomib, using a top concentration of 2.5 μM and 0.039 μM, respectively.

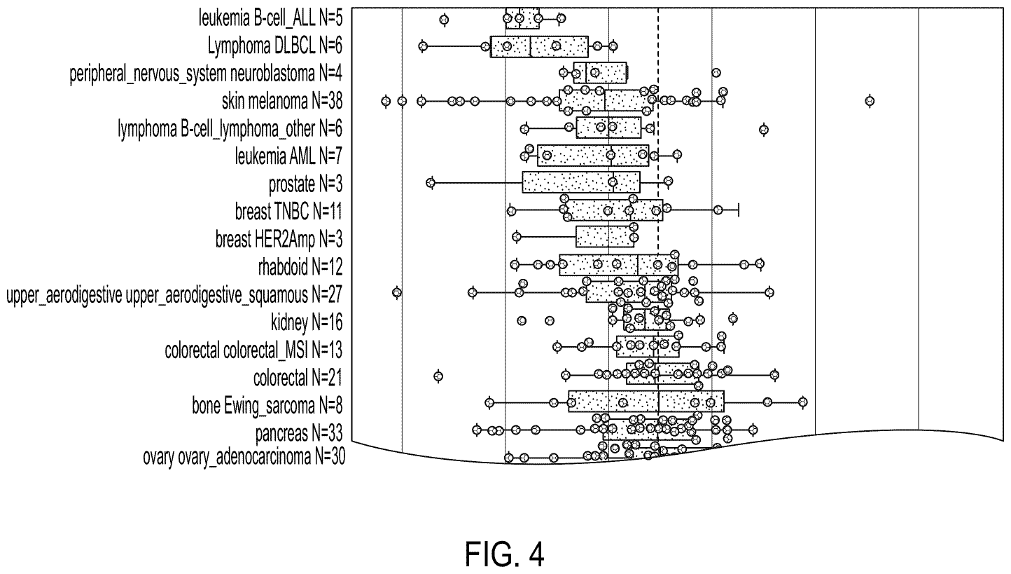

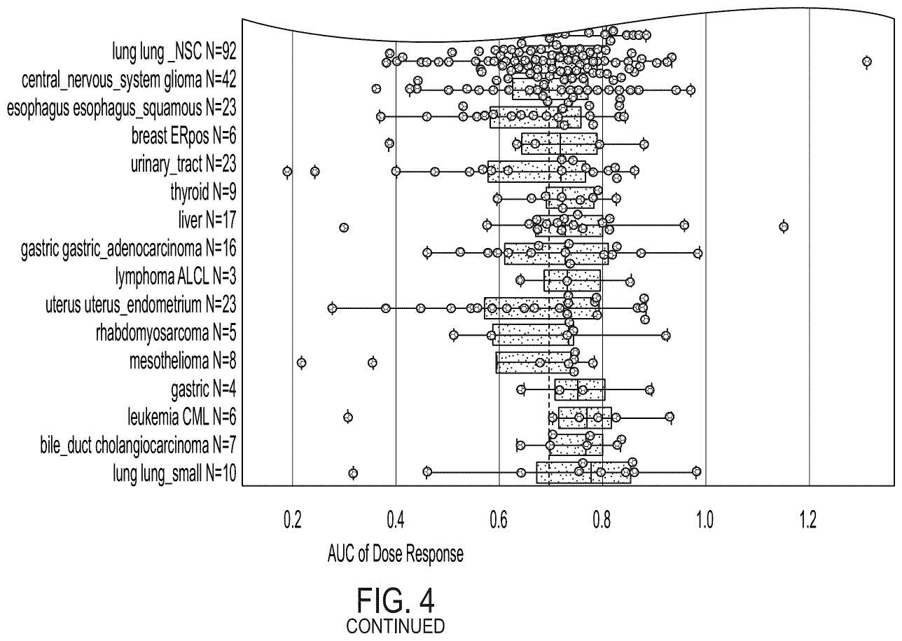

Following 3 days of treatment with compounds, cells were lysed, genomic DNA was extracted, barcodes were amplified by PCR and detected with Next-Generation Sequencing. Cell viability was determined by comparing the counts of cell-line specific barcodes in treated samples to those in the DMSO-control and Day 0 control. Dose-response curves were fit for each cell line and corresponding area under the curves (AUCs) were calculated and compared to the median AUC of all cell lines (FIG. 4). Cell lines with AUCs less than the median were considered most sensitive.

### Example 12. Effects of BRG1/BRM ATPase Inhibitors on the Growth of Uveal Melanoma Cell Lines

Procedure: Uveal melanoma cell lines (92-1, MP41, MP38, MP46) and Non-small cell lung cancer cells (NCIH1299) were plated into 96 well plates with growth media (see Table 6). BRG1/BRM ATPase inhibitor, compound 67, was dissolved in DMSO and added to the cells in a concentration gradient from 0 to 10 micromolar at the time of plating. Cells were incubated at 37° C. for 3 days. After three days of treatment, cell growth was measured with Cell-titer glow (Promega), and luminescence was read on an Envision plate reader (Perkin Elmer).

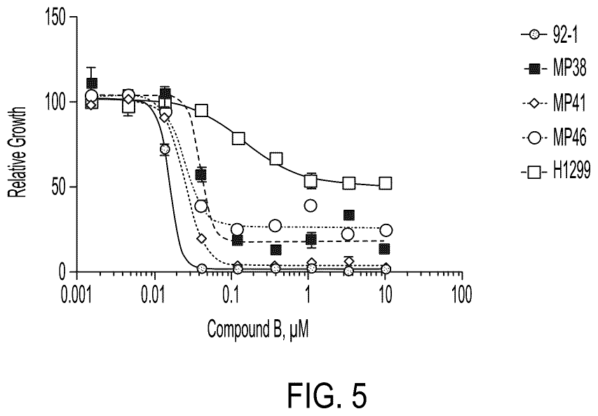

Results: As shown in FIG. 5, Compound B resulted in potent growth inhibition in the uveal melanoma cell lines.

### Example 13. Comparison of BRG1/BRM Inhibitors to Clinical PKC and MEK Inhibitor: In Uveal Melanoma Cell Lines

Procedure: Uveal melanoma cell lines, 92-1 or MP41, were plated in 96 well plates in the presence of growth media (see Table 6). BAF ATPase inhibitor (Compound B), PKC inhibitor (LXS196; MedChemExpress), and MEK Inhibitor (Selumetinib; Selleck Chemicals) were dissolved in DMSO and added to the cells in a concentration gradient from 0 to 10 micromolar at the time of plating. Cells were incubated at 37° C. for 3 days. After three days of treatment, cell growth was measured with Cell-titer glow (Promega), and luminescence was read on an Envision plate reader (Perkin Elmer).

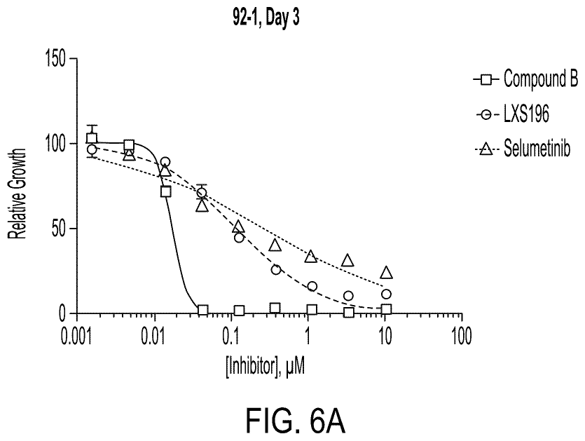

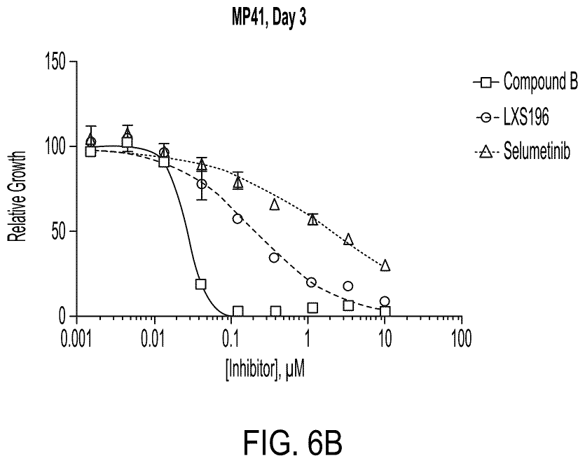

Results: As shown in FIG. 6A and FIG. 6B, Compound B showed more potent effects on growth inhibition of uveal melanoma cells as compared to the clinical PKC and MEK inhibitors. Further, Compound B was found to result in a faster onset of growth inhibition than the clinical PKC and MEK inhibitors.

### Example 14. BRG1/BRM ATPase Inhibitors are Effective at Inhibiting the Growth of PKC Inhibitor-Resistant Cells

Procedure: MP41 uveal melanoma cells were made resistant to the PKC inhibitor (LXS196; MedChemExpress), by long-term culture in growth media (see Table 6) containing increasing concentrations of the compound, up to 1 micromolar. After 3 months, sensitivity of the parental MP41 cells and the PKC inhibitor (PKCi)-resistant cells to the PKC inhibitor (LXS196) or the BRG1/BRM ATPase inhibitor (Compound B) was tested in a 7-day growth inhibition assay as described above in Example 9.

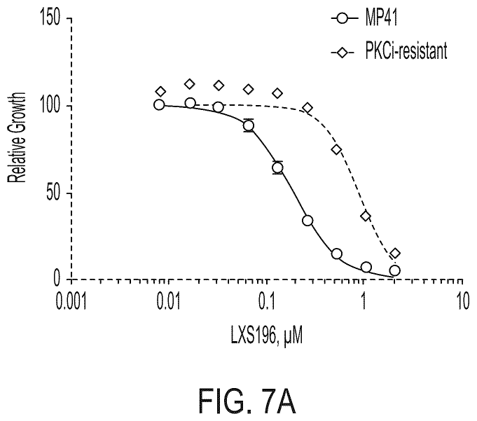

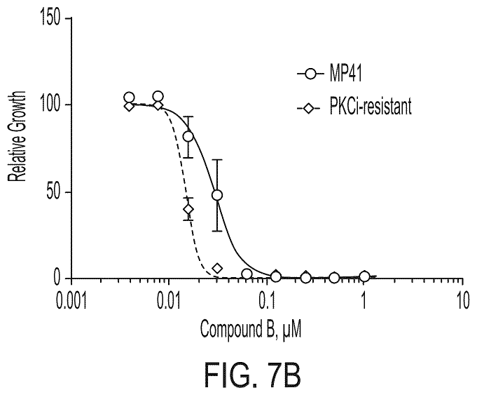

Results: While the PKCi-resistant cells could tolerate growth at higher concentrations of LXS196 than could the parental MP41 cell line (FIG. 7A), the BRG1/BRM ATPase inhibitor (Compound B) still resulted in strong growth inhibition of both the PKCI-resistant and parental cell lines (FIG. 7B). The PKCI-resistant cells were more sensitive to Compound B than were the parental MP41 cells (FIG. 7B).

### Example 15. Synthesis of Compound C

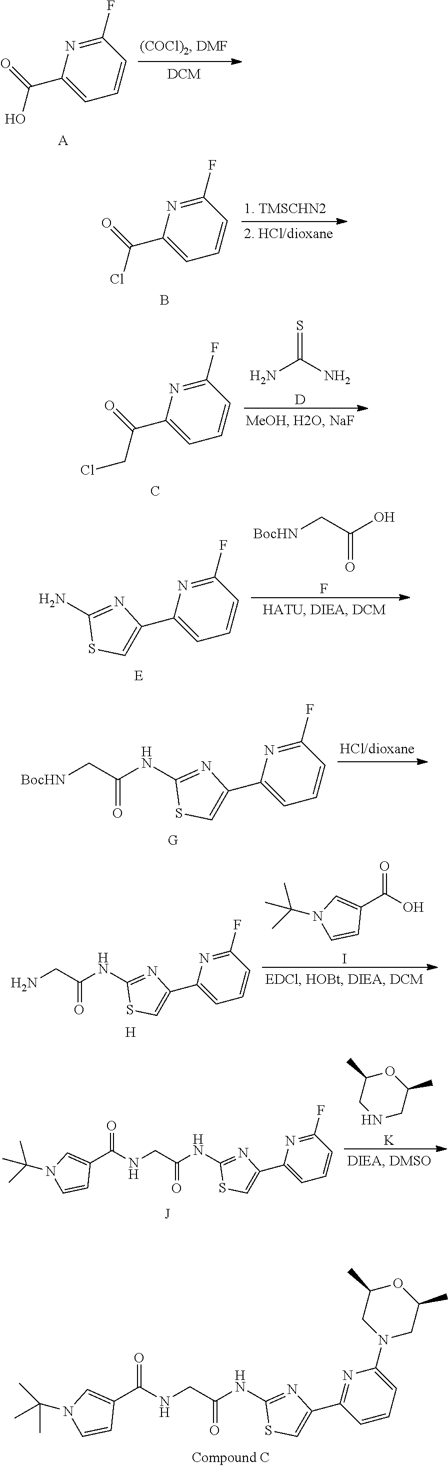

### Step 1. Preparation of β fluoropyridine-2-carbonyl chloride (Intermediate B)

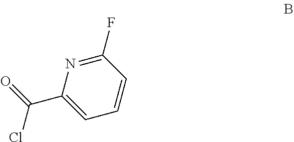

To a cooled (0° C.) solution of 6-fluoropyridine-2-carboxylic acid (50.00 g, 354.36 mmol) in dichloromethane (500 mL) and N,N-dimethylformamide (0.26 mL, 3.54 mmol) was added oxalyl chloride (155.10 mL, 1.77 mol). After complete addition of oxalyl chloride, the reaction mixture was warmed to room temperature and stirred for an additional 0.5 h. The mixture was concentrated under vacuum to give intermediate B (56.50 g) as a white solid, which was used to next step without further purification.

### Step 2. Preparation of 2-chloro-1-(6-fluoro-2-pyridyl)ethenone (Intermediate C)

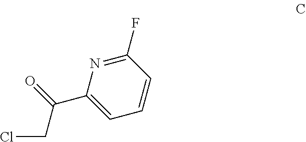 [SMILES: O=C(CCl)c1cccc(F)n1]

To a cooled (0° C.) mixture of Intermediate B (58.00 g, 351.00 mmol) in 1,4-dioxane (800 mL) was added in a dropwise manner a solution of 2 M trimethylsllyl diazomethane in hexanes (351 mL). The resulting reaction mixture was stirred at 25° C. for 10 h. The reaction mixture was subsequently quenched with a solution of 4 M HCl in 1,4-dioxane (500 mL). After stirring for 2 h, the reaction solution was concentrated under vacuum to give an oil. The residue was diluted with saturated aqueous NaHCO₃ (500 mL) and extracted with ethyl acetate (200 mL×3). The combined organic layers were washed with brine (300 mL×2), dried over Na₂SO₄, filtered, and concentrated under reduced pressure to give Intermediate C (35.50 g) as a white solid, which was used to next step directly. LCMS (ESI) m/z: [M+H]⁺=173.8.

### Step 3. Preparation of 4-(6-fluoro-2-pyridyl)thiazol-2-amine (Intermediate E)

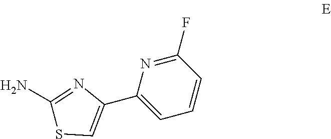 [SMILES: Nc1nc(-c2cccc(F)n2)cs1]

To a solution of Intermediate C (35.50 g, 204.53 mmol) and thiourea (14.01 g, 184.07 mmol) in a mixture of MeOH (250 mL) and H₂O (250 mL) at room temperature was added NaF (3.56 g, 84.82 mmol). After stirring for 0.5 h, the reaction mixture was partially concentrated under vacuum to remove MeOH, and the resulting solution was acidified to pH ˜3 with aqueous 2 M HCl. After 15 min, the solution was extracted with ethyl acetate (200 mL×3), the organic layers were discarded and the aqueous phase was alkalized with NaHCO₃ (500 mL) and stirred for 30 min, then extracted with ethyl acetate (325 mL×3), the combined organic layers were washed with brine (225 mL*3), dried over Na₂SO₄, filtered, and concentrated under reduced pressure. The residue was triturated with petroleum ether (300 mL) and stirred at 25° C. for 10 min and filtered. The resultant solids were dried under vacuum to give Intermediate E (28.00 g, 143.43 mmol, 70.13% yield, 100% purity) as a white solid. LCMS (ESI) m/z: [M+H]⁺=195.8; ¹H NMR (400 MHz, DMSO-d₆) δ 8.00-7.96 (m, 1H), 7.72 (d, J=7.2 Hz, 1H), 7.24 (s, 1H), 7.16 (s, 2H), 7.02 (d, J=8.0 Hz, 1H).

### Step 4. Preparation of tert-butyl N-[2-[[4-(6-fluoro-2-pyridyl)thiazol-2-yl]amino]-2-oxo-ethyl]carbamate (Intermediate G)

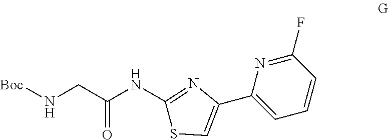 [SMILES: CC(C)(C)OC(=O)NCC(=O)Nc1nc(-c2cccc(F)n2)cs1]

To a solution of N-Boc-glycine (5.92 g, 33.81 mmol), HATU (12.86 g, 33.81 mmol), and DIEA (15.89 g, 122.94 mmol, 21.41 mL) in dichloromethane (100 mL) was added Intermediate E (6.00 g, 30.74 mmol. After stirring for 2 h, the reaction mixture was concentrated and subsequently diluted with water (100 mL) and extracted with ethyl acetate (80 mL×4). The combined organic layers were washed with brine (100 mL×2), dried over Na₂SO₄, filtered, and concentrated under reduced pressure to give a residue. The residue was triturated with a 1:1 mixture of petroleum ether and MeOH (40 mL). After stirring at 25° C. for 20 min, the suspension was filtered, the filter cake was washed with MTBE (20 mL), and dried in vacuo to give Intermediate O (7.7 g, 21.63 mmol, 70.4% yield, 99.0% purity) as a white solid. LCMS (ESI) m/z: [M+H]⁺=353.1.

### Step 5. Preparation of 2-((4-(6-fluoropyridin-2-yl(thiazol-2-yl)amino)-2-oxoethan-1-aminium chloride (Intermediate H)

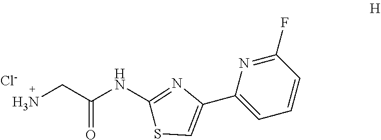 [SMILES: [Cl-] ; NCC(=O)Nc1nc(-c2cccc(F)n2)cs1]

A solution of Intermediate O (5.40 g, 15.32 mmol) in 4 M HCl in 1,4-dioxane (35 mL) was stirred at 25° C. for 1.5 h. The mixture was concentrated under vacuum to give Intermediate H (4.42 g) as a white solid, which was used to next step directly without further purification. LCMS (ESI) m/z: [M+H]⁺ 252.9.

### Step 6. Preparation of 1-tert-butyl-N-[2-[[4-(6-fluoro-2-pyridyl)thiazol-2-yl]amino]-2-oxo-ethyl]pyrrole-3-carboxamide (Intermediate J)

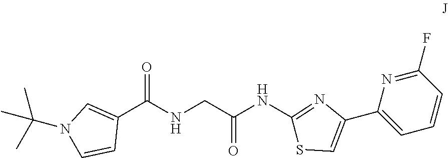 [SMILES: CC(C)(C)n1ccc(C(=O)NCC(=O)Nc2nc(-c3cccc(F)n3)cs2)c1]

To a solution of Intermediate H (3.00 g, 10.39 mmol), 1-tert-butylpyrrole-3-carboxylic acid (1.74 g, 10.39 mmol), and DIEA (6.71 g, 51.95 mmol, 9.05 mL) in dichloromethane (40 mL) was sequentially added HOBt (1.68 g, 12.47 mmol) and EDCl (2.39 g, 12.47 mmol). After stirring for 4 h, the mixture was concentrated under vacuum. The residue was diluted with water (250 mL) and extracted with ethyl acetate (200 mL×3). The combined organic layers were washed with brine (300 mL×3), dried over Na₂SO₄, filtered, and concentrated under reduced pressure. The resulting solids were triturated with a 1:1 mixture of MTBE/ethyl acetate (400 mL) and after 30 min, the suspension was filtered. The solids were washed with MTBE (85 mL×3) and then dried under vacuum to give Intermediate J (3.10 g, 7.64 mmol, 73.8% yield, 99.0% purity) as a white solid.

LCMS (ESI) m/z: [M+H]⁺=402.3.

¹H NMR (400 MHz, DMSO-d₆) δ 12.40 (s, 1H), 8.18-8.15 (m, 1H), 8.09-8.08 (m, 1H), 7.87-7.83 (m, 2H), 7.52 (s, 1H), 7.11 (d, J=8.0 Hz, 1H), 6.97 (m, 1H), 6.47 (s, 1H), 4.10 (d, J=5.6 Hz, 2H), 1.49 (s, 9H).

### Step 7. Preparation of 1-(tert-butyl)-N-(2-((4-(6-(cis-2,6-dimethylmorpholino)pyridin-2-yl)thiazol-2-yl)amino)-2-oxoethyl)-1H-pyrrole-3-carboxamide (Compound C)

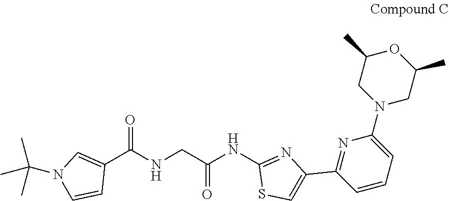 [SMILES: C[C@@H]1CN(c2cccc(-c3csc(NC(=O)CNC(=O)c4ccn(C(C)(C)C)c4)n3)n2)C[C@H](C)O1]

To a solution of Intermediate J (0.100 g, 0.249 mmol) in DMSO (1 mL) was added DIEA (0.130 mL, 0.747 mmol) and cis-2,6-dimethylmorpholine (0.057 g, 0.498 mmol) and the mixture was stirred at 120° C. After 12 h, the solution was cooled to room temperature and reaction mixture was diluted with MeOH (3 ml). The residue was purified by prep-HPLC (0.1% TFA; column: Luna C18 150*25 5 u; mobile phase: [water (0.075% TFA)-ACN]; B %: 30%-60%, 2 min). The appropriate fractions were collected and lyophilized to give Compound C (0.079 g, 0.129 mmol, 51.94% yield, 100% purity) as a white solid. LCMS (ESI) m/z: [M+H]⁺=497.5.

¹H NMR (400 MHz, DMSO-d₆) δ 12.27 (s, 1H), 8.17-8.14 (m, 1H), 7.75 (s, 1H), 7.63-7.59 (m, 1H), 7.51 (s, 1H), 7.25 (d, J=7.2 Hz, 1H), 6.98 (s, 1H), 8.79 (d, J=8.8 Hz, 1H), 6.47 (s, 1H), 4.24 (d, J=12.4 Hz, 2H), 4.08 (d, J=5.8 Hz, 2H), 3.84-3.81 (m, 2H), 2.44-2.38 (m, 2H), 1.49 (s, 9H), 1.18 (d, J=5.8 Hz, 8H).

### Example 16. BRG1/BRM ATPase Inhibitors Cause Uveal Melanoma Tumor Growth Inhibition in Vivo

Procedure: Nude mice (Envigo) were engrafted subcutaneously in the axillary region with 5×10⁶ 92-1 uveal melanoma cells in 50% Matrigel. Tumors were grown to a mean of ˜200 mm³, at which point mice were grouped and dosing was initiated. Mice were dosed once daily by oral gavage with vehicle (20% 2-Hydroxypropyl-8-Cyclodextrin) or increasing doses of Compound C. Tumor volumes and body weights were measured over the course of 3 weeks, and doses were adjusted by body weight to achieve the proper dose in terms of mg/kg. At this time, animals were sacrificed, and tumors were dissected and imaged.

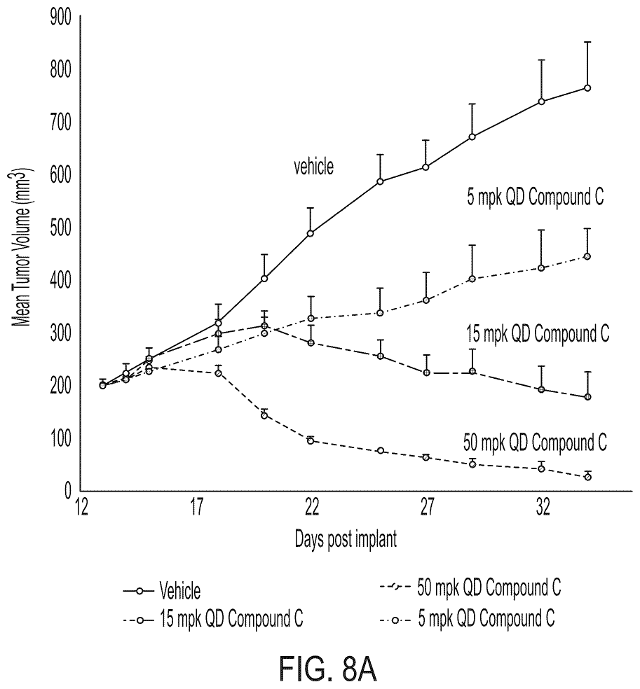

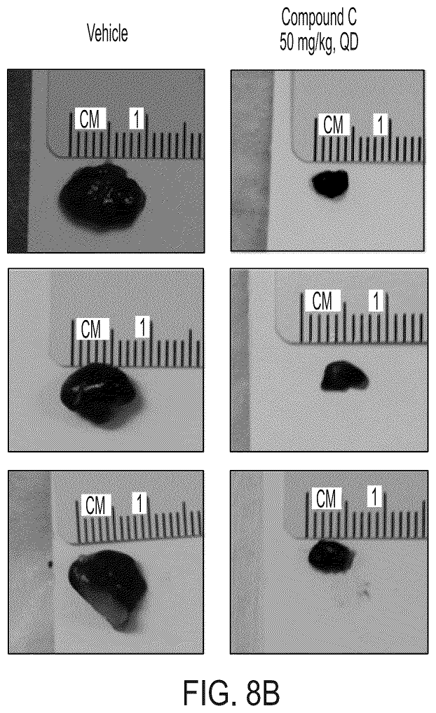

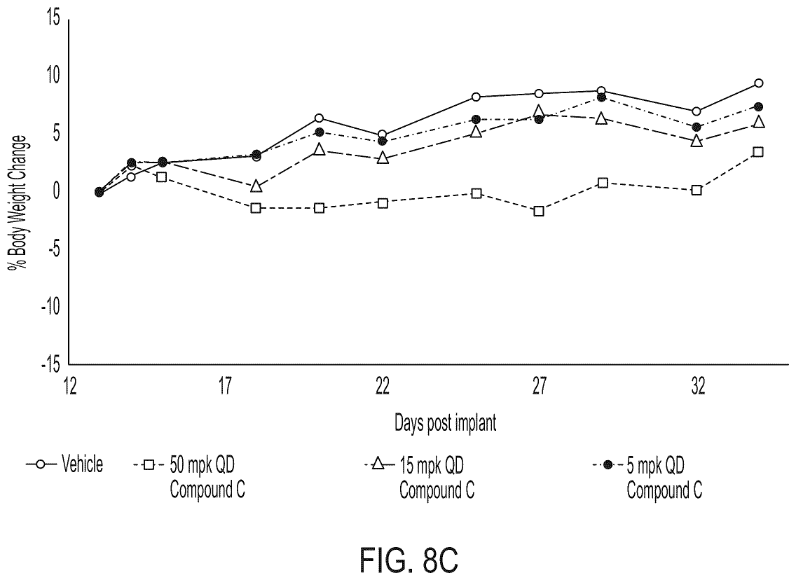

Results: Treatment with Compound C led to tumor growth inhibition in a dose-dependent manner with tumor regression observed at the highest (50 mg/kg) dose. (FIG. 8A and FIG. 8B). All treatments were well tolerated with no body weight loss observed (FIG. 8C).

### OTHER EMBODIMENTS

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A compound having the structure: 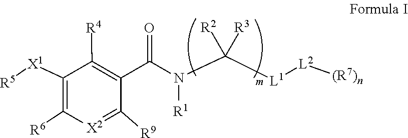
wherein
m is 0, 1, 2, or 3;
n is 0, 1, 2, 3, or 4;
X¹ is —S—, —SO—, —SO₂—, or —S(O)(NH)—;
X² is N or CR⁸;
R¹ is hydrogen or optionally substituted C₁-C₆ alkyl;
each R² and each R³ are independently hydrogen, optionally substituted C₁-C₆ alkyl, or optionally substituted C₁-C₆ heteroalkyl;
L¹ is optionally substituted 9- or 10-membered bicyclic heterocyclyl or optionally substituted 9- or 10-membered bicyclic heteroaryl;
L² is absent, optionally substituted C₃-C₁₀ cycloalkyl, optionally substituted C₆-C₁₀ aryl, optionally substituted 5- to 14-membered heteroaryl, or optionally substituted 4- to 14-membered heterocyclyl;
R⁴ is hydrogen, halo, optionally substituted C₁-C₆ alkyl, or optionally substituted C₃-C₁₀ cycloalkyl;
R⁵ is optionally substituted C₁-C₆ alkyl, optionally substituted C₁-C₆ heteroalkyl, or optionally substituted amino, and R⁶ is hydrogen, halo, cyano, optionally substituted C₁-C₆ alkyl, optionally substituted C₂-C₆ alkenyl, or optionally substituted C₃-C₁₀ cycloalkyl; or R⁵ and R⁶, together with the atoms to which they are attached, combine to form an optionally substituted 5- to 8-membered heterocyclyl;
each R¹ is independently optionally substituted C₁-C₆ alkyl, optionally substituted C₁-C₆ heteroalkyl, halo, optionally substituted C₃-C₁₀ cycloalkyl, optionally substituted C₃-C₁₀ cycloalkyl C₁-C₆ alkyl, optionally substituted 5- to 14-membered heteroaryl, optionally substituted 4- to 14-membered heterocyclyl, —N(R^{7A})₂, or —OR^{7A}, wherein each R^{7A} is independently H, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted 5- to 10-membered heteroaryl, or optionally substituted 4- to 10-membered heterocyclyl, or two geminal R^{7A} groups, together with the atom to which they are attached, combine to form optionally substituted 5- to 10-membered heteroaryl or optionally substituted 4- to 10-membered heterocyclyl; or two geminal R⁷ groups, together, with the atom to which they are attached, combine to form carbonyl;
R⁸ is hydrogen, halo, optionally substituted C₁-C₆ alkyl, or optionally substituted C₃-C₁₀ cycloalkyl; and
R⁹ is hydrogen or halo;
or a pharmaceutically acceptable salt thereof.

2.-9. (canceled)

10. The compound of claim 1, or a pharmaceutically acceptable salt thereof, wherein 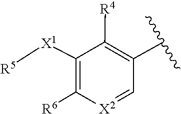
is a group of the following structure 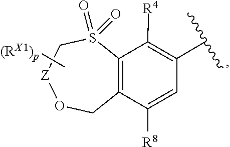
wherein
Z is CH₂, CO, or C(R^{X2})₂;
each R^{X1} is independently optionally substituted C₁-C₆ alkyl or halo, or two geminal R^{X1} groups, together with the atom to which they are attached, combine to form a carbonyl;
each R^{X2} is independently H or optionally substituted C₁-C₆ alkyl; and
p is 0, 1, 2, 3, or 4.

11.-14. (canceled)

15. The compound of claim 1, or a pharmaceutically acceptable salt thereof, wherein R⁸ is hydrogen or halo.

16. (canceled)

17. The compound of claim 1, or a pharmaceutically acceptable salt thereof, wherein X² is N.

18.-20. (canceled)

21. The compound of claim 1, or a pharmaceutically acceptable salt thereof, wherein at least one R^{X1} is optionally substituted C₁-C₆ alkyl, or at least one R^{X1} is halo, or wherein at least two geminal R^{X1} groups, together with the atom to which they are attached, combine to form a carbonyl.

22.-23. (canceled)

24. The compound of claim 1, or a pharmaceutically acceptable salt thereof, wherein L¹ is 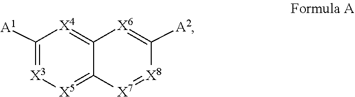
wherein
each of X³, X⁴, X⁵, X⁶, X⁷, and X⁸ is independently N or CR^{L1};
each R^{L1} is independently H, halo, optionally substituted C₁-C₆ alkyl;
A¹ is a bond to —(C(R²)(R³))ₘ—; and
A² is a bond to L².

25. The compound of claim 24, or a pharmaceutically acceptable salt thereof, wherein L¹ is

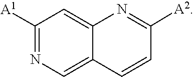

26.-31. (canceled)

32. The compound of claim 24, or a pharmaceutically acceptable salt thereof, wherein -L²-(R⁷)ₙ is a group of the following structure:

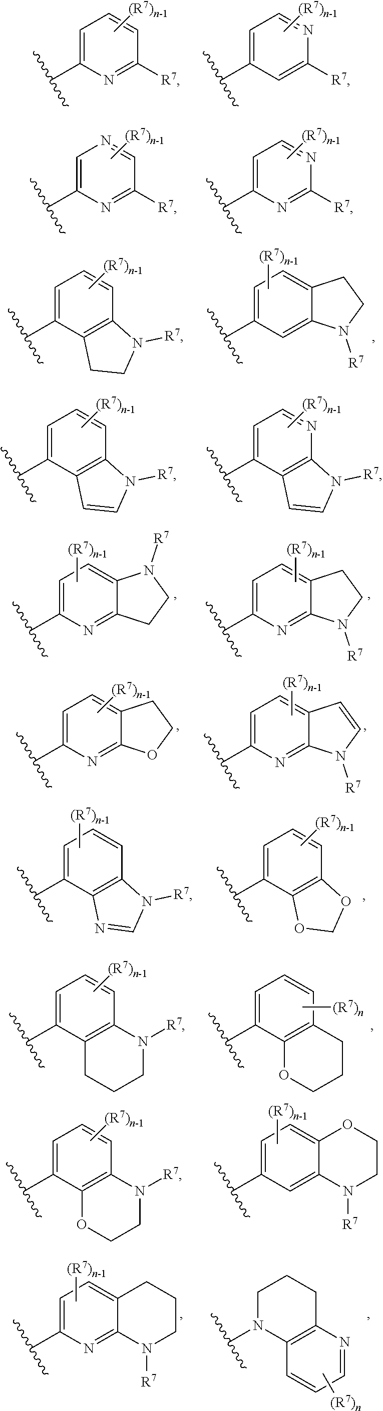

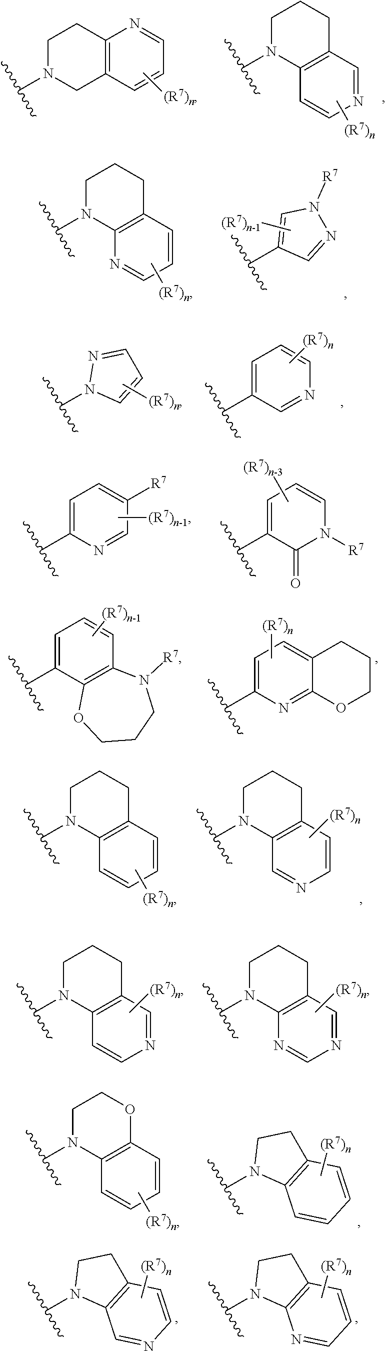

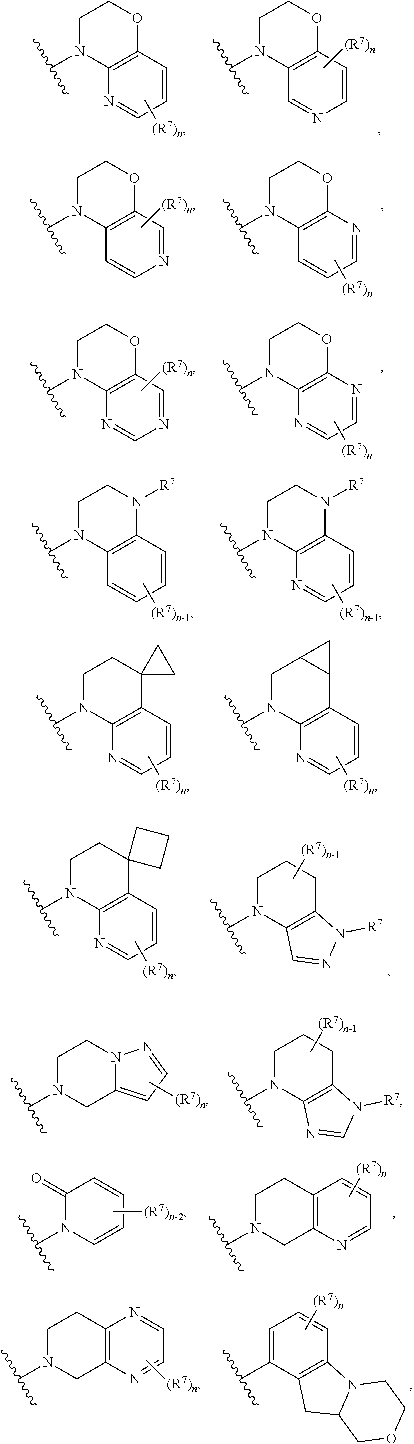

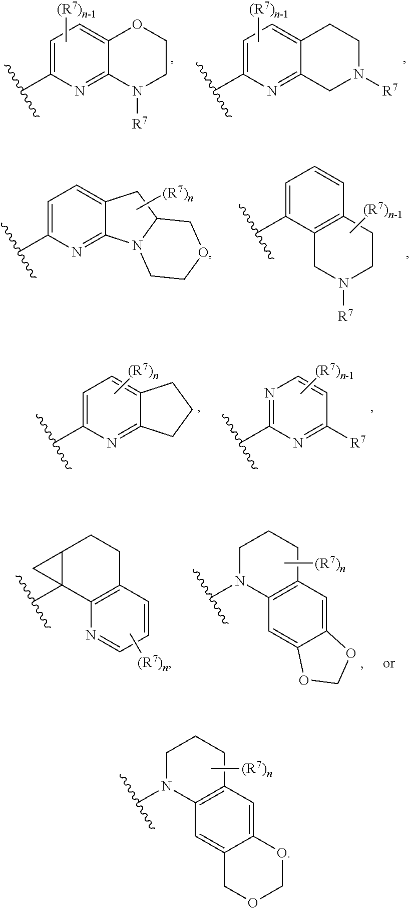

33. (canceled)

34. The compound of claim 32, or a pharmaceutically acceptable salt thereof, wherein -L²-(R⁷)ₙ is a group of the following structure:

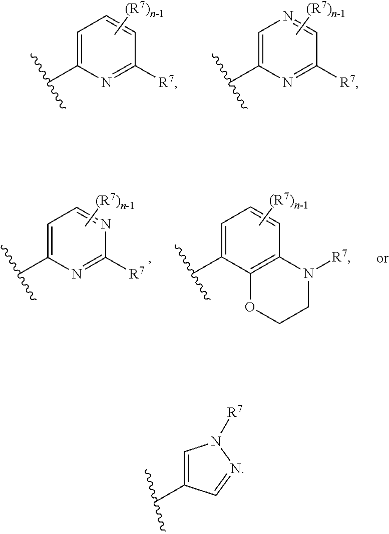

35.-39. (canceled)

40. The compound of claim 32, or a pharmaceutically acceptable salt thereof, wherein -L²-(R⁷)ₙ is a group of the following structure:

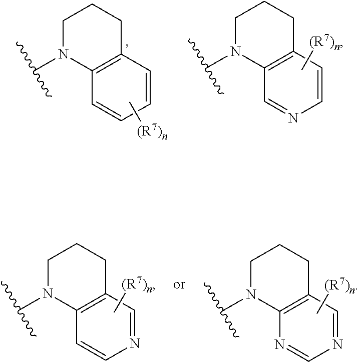

41. The compound of claim 32, or a pharmaceutically acceptable salt thereof, wherein -L²-(R⁷)ₙ is a group of the following structure:

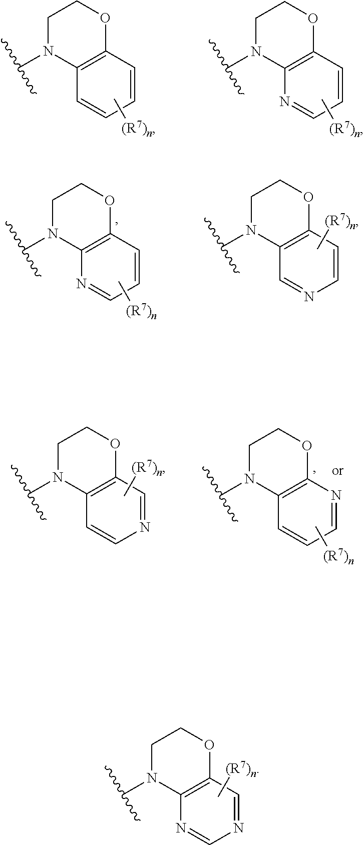

42. The compound of claim 32, or a pharmaceutically acceptable salt thereof, wherein L² is optionally substituted C₆-C₁₀ aryl.

43.-57. (canceled)

58. The compound of claim 32, or a pharmaceutically acceptable salt thereof, wherein at least one R⁷ is cyclopropyl, 2,2-difluorocyclopropyl, difluoromethoxy, 2,6-dimethylmorpholin-4-yl, N-azetidinyl, 3-fluorocyclobutyl, 2-methoxyethyl, ethoxy, methoxy, 2,2-difluoroethoxy, 2,2-difluoroethyl, trifluoromethyl, isopropyl, methyl, acetyl, fluoro, chloro, 1-methylpyrazol-3-yl, dimethylamino, N-methyl-N-(2-methoxyethyl)-amino, N-ethyl-N-(2-methoxyethyl)-amino, N-(2-propyl)-N-(2-methoxyethyl)-amino, 2-methoxyethylamino, 3-aza-8-oxa-bicyclo[4.3.0]non-3-yl, 3-aza-7-oxa-bicyclo[4.3.0]non-3-yl, 1-fluorocyclobut-1-yl, 3-fluoropyrrolidin-1-yl, 3-methoxypyrrolidin-1-yl, oxetan-3-yl, N-methylindolin-4-yl, 2,2-difluoro-3-methylcycloprop-1-yl, 3-methoxyazetidin-1-yl, 3-methoxypiperidin-1-yl, 1,2-dimethyl-7-azaindol-4-yl, 1-methyl-7-azaindol-4-yl, 2,3-methylenedioxyphenyl, N-methyl-N-(3-oxetanyl)amino, 3-oxetanyloxy, 1,1-difluoro-5-azaspiro[2.3]hex-5-yl, 1-fluoromethyl-cyclopropyl, N-(3-tetrahydrofuranyl)methylamino, N-indolinyl, N-1,4-oxazepanyl, 2-fluoro-2-propyl, 1,1-difluoro-2-propyl, 2,2-difluoro-1-methylcycloprop-1-yl, 1-methylcyclopropyl, 4,4-difluoropiperidin-1-yl, 2-methoxyethoxy, 3,3-difluorocyclobut-1-yl, N-methyl-N-1-methoxyprop-2-ylamino, 1-methoxyprop-2-ylamino, 1-methoxyethyl, 4-methylpiperazinyl, 3-methylmorpholinyl, 2,2-difluoropropoxy, 3-methoxycyclobutyl, methylamino, 4-dimethylamino-3,3-difluoropiperidinyl, 4-methylamino-3,3-difluoropiperidinyl, 3,3-difluoropyrrolidinyl, N-methyl-N-3-methoxycyclobutylamino, 1-methylpyrazol-5-yl, 6-oxa-3-azabicyclo[3.1.1]hept-3-yl, cyclopropyloxy, 2,6-dimethylpyrid-4-yl, 2-methylpyrrolidinyl, 4-oxabicyclo[4.1.0]hept-1-yl, N-methyl-N-(2,6-dimethyltetrahydropyran-4-yl)amino, or N-methyl-N-3-methyloxetan-3-ylmethylamino.

59. The compound of claim 1, or a pharmaceutically acceptable salt thereof, wherein R¹ is hydrogen.

60. A compound selected from the group consisting of compounds 1-308 and pharmaceutically acceptable salts thereof.

61. A compound selected from the group consisting of compounds 309-856 and pharmaceutically acceptable salts thereof.

62. The compound of claim 1, or a pharmaceutically acceptable salt thereof, wherein the compound has a ratio of BRG1 IC₅₀ to BRM IC₅₀ of at least 5.

63.-68. (canceled)

69. A pharmaceutical composition comprising a compound of claim 1 and a pharmaceutically acceptable excipient.

70.-71. (canceled)

72. A method of treating a BAF complex-related disorder in a subject in need thereof, the method comprising administering to the subject an effective amount of a compound of claim 1.

73. The method of claim 72, wherein the BAF complex-related disorder is cancer or a viral infection.

74.-80. (canceled)

81. The method of claim 73, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

82.-117. (canceled)
